(19) United States
(12) Patent Application Publication
(10) Pub. No.: US 2026/0004885 A1
(43) Pub. Date: Jan. 1, 2026

(54) BIOMARKERS FOR DETERMINING A CANCER DISEASE STATE, RESPONSE TO IMMUNO-ONCOLOGY, STAGES OF FIBROSIS IN NON-ALCOHOLIC STEATOHEPATITIS, OR APPLICATION OF AGE OR SEX RELATED BIOMARKER PANEL FOR QUALITY CONTROL

(71) Applicant: VENN BIOSCIENCES CORPORATION, South San Francisco,, CA (US)

(72) Inventors: Chirag DHAR, San Mateo, CA (US); Paul Oluwaseyi AIYETAN, Frederick, MD (US); Monil Dilip GANDHI, San Jose, CA (US); Alan Nicolas MITCHELL, Fresno, CA (US); Chad Eagle PICKERING, San Mateo, CA (US); Prasanna RAMACHANDRAN, Menlo Park, CA (US); Daniel SERIE, San Francisco, CA (US); Apoorva SRINIVASAN, San Francisco, CA (US); Gege XU, Redwood City, CA (US)

(21) Appl. No.: 18/844,541

(22) PCT Filed: Apr. 1, 2023
(86) PCT No.: PCT/US2023/065248

§ 371 Date: Sep. 6, 2024

Related U.S. Application Data
(60) Provisional application No. 63/326,689, filed on Apr. 1, 2022.
(60) Provisional application No. 63/327,305, filed on Apr. 4, 2022.
(60) Provisional application No. 63/333,861, filed on Apr. 22, 2022.
(60) Provisional application No. 63/338,225, filed on May 4, 2022.
(60) Provisional application No. 63/364,467, filed on May 10, 2022.
(60) Provisional application No. 63/356,458, filed on Jun. 28, 2022.
(60) Provisional application No. 63/392,812, filed on Jul. 27, 2022.
(60) Provisional application No. 63/376,053, filed on Sep. 16, 2022.
(60) Provisional application No. 63/377,841, filed on Sep. 30, 2022.
(60) Provisional application No. 63/377,850, filed on Sep. 30, 2022.
(60) Provisional application No. 63/485,876, filed on Feb. 17, 2023.
(60) Provisional application No. 63/489,712, filed on Mar. 10, 2023.
(60) Provisional application No. 63/491,241, filed on Mar. 20, 2023.

(51) Int. Cl. G16B 40/20; G16H 10/40; G16H 15/00; G16H 20/00; G16H 50/30

(52) U.S. Cl. CPC G16B 40/20; G16H 10/40; G16H 15/00; G16H 20/00; G16H 50/30

(57) ABSTRACT

Provided herein are methods, devices, and kits for identifying glycosylated polypeptide biomarkers and signatures for progression of a disease or a condition, such as cancer or NASH, or and response of the disease or condition to a treatment. Also provided herein are: i) methods of generating and analyzing glycosylated polypeptide biomarkers, ii) methods of validating a model using glycosylated polypeptides for predicting the disease or condition or for making treatment recommendation, iii) systems and methods for implementing QC of a cohort of samples by analyzing peptide structure data for each sample using a machine learning model to generate a predicted age and/or sex associated for each sample. The quality control issue may include an error of mislabeled samples or an error from sample preparation, or a systemic measurement or an instrument error.

### CROSS-REFERENCE TO RELATED APPLICATIONS

This application claims the priority benefit of U.S. Provisional Patent Application Ser. 63/326,689, filed Apr. 1, 2022, [Attorney Docket No. VENN.P0016US.P1/VENN-00033PR]; U.S. Provisional Patent Application Ser. No. 63/327,305, filed Apr. 4, 2022, [Attorney Docket No. 16653-30019.00/VENN-00034PR]; U.S. Provisional Patent Application Ser. No. 63/333,861, filed Apr. 22, 2022, [Attorney Docket No. VENN.P0017US.P1/VENN-00039PR]; U.S. Provisional Patent Application Ser. No. 63/338,225, filed May 4, 2022, [Attorney Docket No. 16653-30019.01/VENN-00034P1]; U.S. Provisional Patent Application Ser. No. 63/364,467, filed May 10, 2022, [Attorney Docket No. VENN.P0018US.P1/VENN-00038PR]; U.S. Provisional Patent Application Ser. No. 63/356,458, filed Jun. 28, 2022, [Attorney Docket No. 16653-30023.00/VENN-00045PR]; U.S. Provisional Patent Application Ser. No. 63/392,812, filed Jul. 27, 2022 [Attorney Docket No. 16653-30025.00/VENN-00049PR]; U.S. Provisional Patent Application Ser. No. 63/376,053, filed Sep. 16, 2022, [Attorney Docket No. VENN.P0026US.P1/VENN-00052PR]; U.S. Provisional Patent Application Ser. No. 63/377,841, filed Sep. 30, 2022 [Attorney Docket No. VENN.P0022US.P1/VENN-00054PR]; U.S. Provisional Patent Application Ser. No. 63/377,850, filed Sep. 30, 2022, [Attorney Docket No. VENN.P0021US.P1/VENN-00053PR]; U.S. Provisional Patent Application Ser. No. 63/485,876, filed Feb. 17, 2023, [Attorney Docket No. 16653-30023.01/VENN-00045P1]; U.S. Provisional Patent Application Ser. No. 63/489,712, filed Mar. 10, 2023 [Attorney Docket No. VENN.P0026US.P2/VENN-00052P1]; U.S. Provisional Patent Application Ser. No. 63/491,241, filed Mar. 20, 2023, [Attorney Docket No. 16653-30027.00/VENN-00055PR]; which are hereby all incorporated by reference herein in their entirety.

### FIELD

The present disclosure generally relates to methods and systems for diagnosing, characterizing, and/or treating breast cancer, pancreatic cancer, non small cell lung cancer (NSCLC), ovarian cancer, malignant melanoma, a state of a fatty liver disease (FLD) progression. More particularly, the present disclosure relates to analyzing quantification data for a set of peptide structures detected in a biological sample obtained from a subject for use in a diagnostic assessment of the subject's disease state (e.g., healthy, NASH, breast cancer, pancreatic cancer, NSCLC, ovarian cancer, and melanoma) and/or treating the subject. The present disclosure also generally relates to methods, compositions, and systems for analyzing peptide structures for quality control related to biological samples. More particularly, the present disclosure relates to analyzing quantification data for a set of age-related peptide structures and/or a set of sex-related peptide structures detected in biological samples obtained from subjects for use as a quality control procedure for one or more processes. The present disclosure also relates to analyzing quantification data for a set of peptide structures detected in a biological sample obtained from a subject for use in a diagnostic assessment of whether the subject is likely or not likely to benefit from immuno-oncology treatment for melanoma or NSCLC.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes. Further, glycoproteomic analyses has not previously been used to successfully identify a disease state relating to a disease progression.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease processes. The present disclosure addresses this and other needs by combining site-specific glycoprotein analysis with machine learning and advanced mass spectrometry instrumentation to quantitatively analyze peptide structures that are indicative of specific disease states, including, but not limited to, medical conditions encompassed herein. For example, there is a need to use such analysis to diagnose, detect and/or treat pancreatic cancer (PC) or breast cancer (BC) or to predict a risk for BC.

In order to ensure correct sample analysis of a given set of glycopeptides, the steps in the process to produce and analyze the glycopeptides need to be accurate, including at least for enzymatic digestion of the glycoproteins, glycopeptide enrichment, and mass spectrometry characterization of the glycopeptides. Ideally, a multi-step process such as glycoprotein analysis should include a process to provide assurance that the method has not endured any faulty processing and/or human error.

Diagnosing and treating BC currently relies on ultrasound, mammogram, magnetic resonance imaging, and/or tissue biopsy. For example, the standard proteins evaluated using ELISA-based technology include CA 15.3, TRU-QUANT, BRCA1, BRCA2, HER2, and/or CA 27.29 markers. However, evaluations based on these markers may not provide the level of performance desired with respect to predicting or diagnosing BC or identifying a risk for BC. Further, currently available methods for diagnosing BC may be unable to make an early diagnosis of BC. Late diagnosis of BC in patients can lead to negative health outcomes.

An approach that is both non-invasive and includes a low false positive rate while maintaining a high level of accuracy is needed. Additionally, an approach enabling early diagnosis may help reduce negative health outcomes in patients with BC. Thus, it is desirable to have methods and systems capable of addressing one or more of the above-identified issues. Separately, accumulation of fatty deposits in the liver, in the absence of excessive alcohol consumption, is the hallmark of non-alcoholic fatty liver diseases (NAFLD). NAFLD progresses through various stages of fat accumulation from simple steatosis (NAFL) to steatosis and weak inflammation with or without fibrosis, a condition termed non-alcoholic steatohepatitis (NASH), which, in turn, may progress to the development of liver cirrhosis. Currently, the best method for diagnosis of NASH is liver biopsy, which is still invasive and fraught with inter-operator variability. Current diagnostic techniques do not have the accuracy necessary to definitively predict the stage of FLD (e.g., whether a patient just has fat accumulation or NASH). In the present disclosure, the use of circulating serum glycoproteins circulating in blood were utilized to identify a panel of potential prognostic markers that may aid in predicting NASH and stage of liver fibrosis. Knowing a NASH stage of a patient with a high degree of accuracy would allow medical practitioners to customize treatment for individual patients and achieve better outcomes. Thus, it may be desirable to have methods and systems capable of distinguishing between these and healthy states.

Diagnosing and treating PC currently relies on protein assays evaluated using enzyme-linked immunosorbent assay (ELISA)-based technology. For example, the standard proteins evaluated using ELISA-based technology include the CA 19-9 and CEA proteins. However, evaluations based on these proteins may not provide the level of performance desired with respect to predicting or diagnosing PC. Further, currently available methods for diagnosing PC may be unable to make an early diagnosis of PC. Late diagnosis of PC in patients can lead to negative health outcomes.

An approach that is both non-invasive and includes a low false positive rate while maintaining a high level of accuracy is needed. Additionally, an approach enabling early diagnosis may help reduce negative health outcomes in patients with PC. Thus, it may be desirable to have methods and systems capable of addressing one or more of the above-identified issues.

Lung cancer is the most common cause of cancer death worldwide, with an estimated 1.6 million deaths each year. For lung cancer cases in the U.S., more than 80% are attributable to tobacco smoking. Despite being a preventable disease, lung cancer remains one of the most common and lethal cancers globally due to treatment challenges and limited effective therapeutic options.

Most lung cancer patients (about 85%) are diagnosed with non-small cell lung cancer (NSCLC). Diagnosis is usually confirmed with various imaging techniques (e.g., chest x-ray, CT scan) and fluid analyses (e.g., sputum cytology, thoracentesis). If lung cancer is suspected, a biopsy is collected for morphological and molecular sample analysis. Additional diagnostic options are available including an endoscopic ultrasound, bronchoscopy, blood testing, and immunohistochemistry, each having contextual advantages. Early detection and diagnosis of NSCLC are key for effective treatment, yet early-stage symptoms are often missed or mistaken for other illnesses (e.g., pneumonia or a partially collapsed lung). Consequently, the patient may only notice symptoms when the cancer has metastasized regionally in the lungs or across the body, wherein the 5-year survival rate drops substantially, to less than 10%.

In view of the current treatment options, NSCLC is not considered a curable disease once it progresses to stage III (e.g., larger tumor, potential for metastasis). Common treatments for NSCLC include surgery, for healthy individuals, wherein the cancer is physically removed by removing part of the lung (e.g., lobectomy) or removing the whole lung (e.g., pneumonectomy). Platinum-based therapies are common chemotherapy options, wherein platinum-coordination compounds, such as cisplatin (CDDP) and carboplatin (CBDCA are administered in combination with other cancer therapeutics. More recently, immunotherapies and patient-specific therapies have been used to improve treatment for specific NSCLC patient subgroups, but platinum-based therapies remain a therapeutic cornerstone for NSCLC treatment.

These NSCLC therapies can slow cancer progression, but early diagnosis is key for patient treatment and overall survival. Given the global burden and life-threatening consequences of delayed NSCLC detection, early and unambiguous diagnosis and effective treatment strategies are imperative. Under certain circumstances, the diagnosis of NSCLC using a blood sample may be easier to do than performing a diagnostic test that uses a biopsy sample obtained from a lung.

In light of the above, there is a need for improved analytical methods that involve site-specific analysis of glycoproteins to obtain information about protein glycosylation patterns, which can in turn provide quantitative information that can be used to identify disease states. For example, there is a need to use such analysis to diagnose and/or treat ovarian cancer.

Epithelial ovarian cancer (EOC) is currently the second-most common gynecologic malignancy, the leading cause of death from gynecological cancer, and the fourth-leading cause of cancer-related death in women in the United States. Although EOC can be treated effectively with surgery and adjuvant therapies, only about 15-20% of women are diagnosed at early-stage when 5-year survival is greater than 90%. Instead, the majority of EOC cases are diagnosed at late-stage (stage III or IV), with 5-year survival rates between about 15% and 40%. Diagnosing early-stage EOC is impeded by initial clinical signs and symptoms that are generally nonspecific and commonly missed such as, for example, pelvic pain, urinary urgency/frequency, abdominal bloating, early satiety, loss of appetite, and weight loss.

In addition to late diagnosis and consequent under-treatment of serious disease, benign disease is oftentimes unnecessarily over-treated due to the lack of diagnostic tools to determine the nature of pelvic masses. For example, while over 90% of women presenting with a pelvic mass may ultimately undergo surgery, only about 20% are found to have malignant disease.

Thus, an approach that is non-invasive, accurate, and reliable and that enables early diagnosis is needed. An approach enabling early diagnosis may help reduce negative health outcomes in patients with ovarian cancer, reduce the under-treatment of ovarian cancer, and/or reduce the over-treatment of benign disease. In addition, more strategic treatments can be provided with a diagnostic test that can assess whether a subject has early stage or late stage ovarian cancer. Thus, it may be desirable to have methods and systems capable of addressing one or more of the above-identified issues.

In certain situations, there is a desire for improved analytical methods that involve site-specific analysis of glycoproteins to obtain information about protein glycosylation patterns, which can in turn provide quantitative information that can be used to manage the treatment of a subject diagnosed with a particular disease or condition such as melanoma or NSCLC. Thus, it may be desirable to have methods and systems capable of addressing one or more of the above-identified issues.

One cancer whose treatment would benefit from the contemplated glycoproteomic analysis is non-small-cell lung cancer (NSCLC). Subjects diagnosed with NSCLC may undergo pembrolizumab therapy, but objective response rates for pembrolizumab therapy are low in NSCLC patients. Subjects should avoid unnecessary exposure and toxicities if they will not respond to pembrolizumab therapy. Hence, there is a need for determination of likely response to pembrolizumab therapy for NSCLC patients.

### SUMMARY

In various embodiments, there is a method of classifying a biological sample with respect to a plurality of states associated with fatty liver disease (FLD) progression, the method comprising: receiving peptide structure data corresponding to a set of glycoproteins and/or non-glycosylated peptides in the biological sample obtained from a subject; inputting quantification data identified from the peptide structure data for a set of peptide structures into at least one machine learning model, wherein the set of peptide structures includes at least one peptide structure identified from a plurality of peptide structures in Table 1A; analyzing the quantification data using the machine learning model to generate a disease indicator; and generating a diagnosis output based on the disease indicator that classifies the biological sample as evidencing a corresponding state of the plurality of states associated with the FLD progression.

In various embodiments, there is a method of training a model to diagnose a subject with one of a plurality of states associated with non-alcoholic steatohepatitis (NASH) progression, the method may comprise receiving quantification data for a panel of peptide structures for a plurality of subjects, each diagnosed with one of the plurality of states associated with NASH progression, wherein the quantification data comprises a plurality of peptide structure profiles for the plurality of subjects and identifies a corresponding state of the plurality of states for each peptide structure profile of the plurality of peptide structure profiles; and training a machine learning model using the quantification data to determine which state of the plurality of states a biological sample (such as at least one of blood, serum, or plasma) from the subject corresponds.

In various embodiments, there are methods of training a model to detect the presence of non-alcoholic steatohepatitis (NASH) in a subject, the methods comprising receiving quantification data for a panel of peptide structures for a plurality of subjects, each assessed for the presence of NASH, wherein the quantification data comprises a plurality of peptide structure profiles for the plurality of subjects and identifies the presence or absence of NASH for each peptide structure profile of the plurality of peptide structure profiles; and training a machine learning model using the quantification data to determine the presence or absence of NASH in a biological sample (including blood, plasma, or serum) corresponding to the subject.

In various embodiments, there are methods of detecting a presence of one of a plurality of states associated with fatty liver disease (FLD) progression in a biological sample, the method comprising receiving peptide structure data corresponding to a set of glycoproteins and/or non-glycosylated peptides in the biological sample obtained from a subject; analyzing the peptide structure data using at least one supervised machine learning model to generate a disease indicator based on at least 2 peptide structures selected from a group of peptide structures identified in Table 1A; and detecting the presence of a corresponding state of the plurality of states associated with the FLD progression in response to a determination that the disease indicator falls within a selected range associated with the corresponding state.

In various embodiments, methods of classifying a biological sample as corresponding to one of a plurality of states associated with fatty liver disease (FLD) progression are provided, the methods comprising training at least one supervised machine learning model using training data, wherein the training data comprises a plurality of peptide structure profiles for a plurality of training subjects and identifies a state of the plurality of states for each peptide structure profile of the plurality of peptide structure profiles; receiving peptide structure data corresponding to a set of non-glycosylated peptides and/or glycopeptides in the biological sample obtained from a subject; inputting quantification data identified from the peptide structure data for a set of peptide structures into the supervised machine learning model that has been trained, wherein the set of peptide structures includes at least one peptide structure identified in Table 1A; analyzing the quantification data using the supervised machine learning model to generate a score; determining that the score falls within a selected range associated with a corresponding state of the plurality of states associated with the FLD progression; and generating a diagnosis output that indicates that the biological sample evidences the corresponding state, wherein the plurality of states includes a non-alcoholic steatohepatitis (NASH) state or a non-NASH state.

In various embodiments, there are methods of treating a non-alcoholic steatohepatitis (NASH) disorder in a patient to at least one of reduce, stall, or reverse a progression of the NASH disorder into a later stage of NASH, the method comprising: receiving a biological sample from the patient; determining a quantity of at least 2 peptide structures identified in Table 1A in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the patient has the NASH disorder; and administering a therapeutically effective amount of the treatment for NASH.

In various embodiments, there is a method of designing a treatment for a subject diagnosed with a state associated with a fatty liver disease (FLD) progression, the method comprising: designing a therapeutic for treating the subject in response to determining that a biological sample obtained from the subject evidences the state using part or all of any method encompassed herein.

In various embodiments, there is a method of planning a treatment for a subject diagnosed with a state associated with a fatty liver disease (FLD) progression, the method comprising: generating a treatment plan for treating the subject in response to determining that a biological sample obtained from the subject evidences the state using part or all of any method encompassed herein.

In various embodiments, a method of treating a subject diagnosed with a state associated with a fatty liver disease (FLD) progression is provided, the method comprising: administering to the subject a therapeutic to treat the subject based on determining that a biological sample obtained from the subject evidences the state using part or all of any method encompassed herein.

In various embodiments, there is a method of treating a subject diagnosed with a state associated with a fatty liver disease (FLD) progression, the method comprising: selecting a therapeutic to treat the subject based on determining that the subject is responsive to the therapeutic using any method encompassed herein.

In various embodiments, there is a method for analyzing a set of peptide structures in a sample from a patient, the method comprising (a) obtaining the sample from the patient; (b) preparing the sample to form a prepared sample comprising the set of peptide structures; (c) inputting the prepared sample into a mass spectrometry system using a liquid chromatography system; (d) detecting a set of product ions associated with each peptide structure of the set of peptide structures using the mass spectrometry system, wherein the set of peptide structures includes at least one peptide structure selected from peptide structures identified in Table 4; wherein the set of peptide structures includes a peptide structure that is characterized as having: (i) a precursor ion with a mass-charge (m/z) ratio within ±1.5 of the m/z ratio listed for the precursor ion in Table 4 as corresponding to the peptide structure; and (ii) a product ion having an m/z ratio within ±1.0 of the m/z ratio listed for a first product ion in Table 4 as corresponding to the peptide structure; and (e) generating quantification data for the set of product ions using the mass spectrometry system.

In various embodiments, there is a composition comprising at least one of the peptide structures identified in Table 1A. In various embodiments, there are compositions that may comprise a peptide structure or a product ion, wherein: the peptide structure or product ion comprises an amino acid sequence having at least 90% sequence identity to any one of SEQ ID NOS: 1-23, corresponding to peptide structures in Table 1A; and the product ion is selected as one from a group consisting of product ions identified in Table 4 including product ions falling within an identified m/z range. In various embodiments, there is a composition comprising a glycopeptide structure selected as one from a group consisting of peptide structures identified in Table 4, wherein the glycopeptide structure comprises: an amino acid peptide sequence identified in Table 5A as corresponding to the glycopeptide structure; and a glycan structure identified in Table 1A as corresponding to the glycopeptide structure in which the glycan structure is linked to a residue of the amino acid peptide sequence at a corresponding position identified in Table 1A, wherein the glycan structure has a glycan composition. In various embodiments, there is a composition comprising a peptide structure selected as one from a plurality of peptide structures identified in Table 1, wherein: the peptide structure has a monoisotopic mass identified as corresponding to the peptide structure in Table 4; and the peptide structure comprises the amino acid sequence of SEQ ID NOs: 1-23 identified in Table 1A as corresponding to the peptide structure.

In various embodiments, kits may comprise at least one agent for quantifying at least one peptide structure identified in Table 1A to carry out part or all of the methods of any one of claims 1-95. In some embodiments, kits may comprise at least one of a glycopeptide standard, a buffer, or a set of peptide sequences to carry out part or all of the method of any one of claims 1-95, a peptide sequence of the set of peptide sequences identified by a corresponding one of SEQ ID NOS: 1-23, defined in Table 1A.

In various embodiments, systems are provided that may comprise one or more data processors; and a non-transitory computer readable storage medium containing instructions which, when executed on the one or more data processors, cause the one or more data processors to perform part or all of any method encompassed herein. In some embodiments, there are computer-program products tangibly embodied in a non-transitory machine-readable storage medium, including instructions configured to cause one or more data processors to perform part or all of any method encompassed herein.

In various embodiments, there is a method of classifying a sample from an individual suspected of having, known to have, or at risk for having non-alcoholic steatohepatitis (NASH), comprising the step of measuring from the sample for one or more glycopeptides and/or non-glycosylated peptides in Table 1A.

Various embodiments of the disclosure include methods of predicting a stage of fibrosis in non-alcoholic steatohepatitis (NASH) in an individual, comprising the step of measuring from a sample (including blood, serum, or plasma) from the individual for one or more glycopeptides and/or non-glycosylated peptides from Table 1A.

In various embodiments, a computer-program product tangibly embodied in a non-transitory machine-readable storage medium is provided, including instructions configured to cause one or more data processors to perform part or all of any one or more of the methods disclosed herein.

In one aspect, a method for diagnosing a subject or measuring a risk prediction or early detection with respect to a breast cancer (BC) disease state is described in accordance with various embodiments. In various embodiments, the method includes receiving peptide structure data (which may also be referred to as quantification data) corresponding to a biological sample obtained from the subject. In particular embodiments, minimally invasive liquid biopsies, including at least blood-based biopsies, are useful to provide information for a BC disease state. In various embodiments, the method includes analyzing the peptide structure data using at least one supervised machine learning model to generate a disease indicator that indicates whether the biological sample evidences a BC disease state based on at least 1 peptide structure selected from a group of peptide structures identified in Table 9. In various embodiments, the group of peptide structures in Table 9 is associated with the BC disease state. In various embodiments, the group of peptide structures is listed in Table 9 with respect to relative significance to the disease indicator. In various embodiments, the method includes generating a diagnosis output based on the disease indicator.

In one aspect, a method of training a model to diagnose a subject with respect to a breast cancer (BC) disease state is described in accordance with various embodiments. In various embodiments, the method includes receiving quantification data (which may also be referred to as peptide structure data) for a panel of peptide structures for a plurality of subjects. In various embodiments, the plurality of subjects includes a first portion diagnosed with a negative diagnosis of a BC disease state and a second portion diagnosed with a positive diagnosis of the BC disease state. In various embodiments, the quantification data comprises a plurality of peptide structure profiles for the plurality of subjects. In various embodiments, the method includes training a machine learning model using the quantification data to diagnose a biological sample with respect to the BC disease state using a group of peptide structures associated with the BC disease state. In various embodiments, the group of peptide structures is identified in Table 9. In various embodiments, the group of peptide structures is listed in Table 9 with respect to relative significance to diagnosing the biological sample.

In one aspect, a method of monitoring a subject for a breast cancer (BC) disease state is described in accordance with various embodiments. In various embodiments, the method includes receiving first peptide structure data for a first biological sample obtained from a subject at a first timepoint. In various embodiments, the method includes analyzing the first peptide structure data using a supervised machine learning model to generate a first disease indicator based on at least 1 peptide structure selected from a group of peptide structures identified in Table 9, wherein the group of peptide structures in Table 9 comprises a group of peptide structures associated with a BC disease state. In various embodiments, the method includes receiving second peptide structure data of a second biological sample obtained from the subject at a second timepoint. In various embodiments, the method includes analyzing the second peptide structure data using the supervised machine learning model to generate a second disease indicator based on the at least 1 peptide structure selected from the group of peptide structures identified in Table 9. In various embodiments, the method includes generating a diagnosis output based on the first disease indicator and the second disease indicator.

In one aspect, a composition comprising at least one of peptide structures PS-30 through PS-47 identified in Table 9 is described according to various embodiments.

In one aspect, a composition comprising at least one of peptide structures PS-33, PS-42, PS-44, PS-30, PS-47, PS-43, or PS-37 identified in Table 10A is described according to various embodiments.

In one aspect, a composition comprising at least one of peptide structures PS-42, PS-44, PS-41, PS-43, PS-47, PS-37, PS-30, or PS-45 identified in Table 10B is described according to various embodiments.

In one aspect, a composition comprising a peptide structure or a product ion is described according to various embodiments. In various embodiments, the peptide structure or the product ion comprises an amino acid sequence having at least 90% sequence identity to any one of SEQ ID NOS: 46-62, corresponding to peptide structures PS-30 through PS-47 in Table 9. In various embodiments, the product ion is selected as one from a group consisting of product ions identified in Table 11 including product ions falling within an identified m/z range.

In one aspect, a composition comprising a glycopeptide structure selected as one from a group consisting of peptide structures PS-30 through PS-47 identified in Table 9 according to various embodiments. In various embodiments, the glycopeptide structure comprises an amino acid peptide sequence identified in Table 12 as corresponding to the glycopeptide structure and a glycan structure identified in Table 14 as corresponding to the glycopeptide structure in which the glycan structure is linked to a residue of the amino acid peptide sequence at a corresponding position identified in Table 9. In various embodiments, the glycan structure has a glycan composition.

In one aspect, a composition comprising a peptide structure selected as one from a plurality of peptide structures identified in Table 9 according to various embodiments. In various embodiments, the peptide structure has a monoisotopic mass identified as corresponding to the peptide structure in Table 9. In various embodiments, the peptide structure comprises the amino acid sequence of SEQ ID NOs: 46-62 identified in Table 9 as corresponding to the peptide structure.

In one aspect, a composition comprising a peptide structure or a product ion is described according to various embodiments. In various embodiments, the peptide structure or the product ion comprises an amino acid sequence having at least 90% sequence identity to any one of SEQ ID NOS: 46-62. In various embodiments, the product ion is selected as one from a group consisting of product ions identified in Table 11 including product ions falling within an identified m/z range.

In one aspect, a composition comprising a glycopeptide structure selected as one from a group consisting of peptide structures PS-30 through PS-47 identified in Table 9 is described according to various embodiments. In various embodiments, the glycopeptide structure comprises an amino acid peptide sequence identified in Table 12 as corresponding to the glycopeptide structure. In various embodiments, a glycan structure identified in Table 14 as corresponding to the glycopeptide structure in which the glycan structure is linked to a residue of the amino acid peptide sequence at a corresponding position identified in Table 9. In various embodiments, the glycan structure has a glycan composition.

In one aspect, a composition comprising a peptide structure selected as one of PS-30 through PS-47 peptide structures identified in Table 9 is described according to various embodiments. In various embodiments, the peptide structure has a monoisotopic mass identified as corresponding to the peptide structure in Table 9. In various embodiments, the peptide structure comprises the amino acid sequence of SEQ ID NOS: 46-62 identified in Table 9 as corresponding to the peptide structure.

In one aspect, a kit comprising at least one agent for quantifying at least one peptide structure identified in Table 9 to carry out part or all of any one or more of the methods described herein.

In one aspect, a kit comprising at least one agent for quantifying at least one peptide structure identified in Table 10A or 10B to carry out part or all of any one or more of the methods described herein.

In one aspect, a kit comprising at least one of a glycopeptide standard, a buffer, or a set of peptide sequences to carry out part or all of any one or more of the methods described herein, a peptide sequence of the set of peptide sequences identified by a corresponding one of SEQ ID NOS: 46-62, defined in Table 9 is described according to various embodiments.

In one aspect, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one aspect, a computer-program product tangibly embodied in a non-transitory machine-readable storage medium, including instructions configured to cause one or more data processors to perform part or all of any one or more of the methods described herein.

In one aspect, a method for diagnosing a subject with respect to a pancreatic cancer (PC) disease state is described in accordance with various embodiments. In various embodiments, the method includes receiving peptide structure data (which may also be referred to as quantification data) corresponding to a biological sample obtained from the subject. In various embodiments, the method includes analyzing the peptide structure data using at least one supervised machine learning model to generate a disease indicator that indicates whether the biological sample evidences a PC disease state based on at least 1 peptide structure selected from a group of peptide structures identified in Table 16. In various embodiments, the group of peptide structures in Table 16 is associated with the PC disease state. In various embodiments, the group of peptide structures is listed in Table 16 with respect to relative significance to the disease indicator. In various embodiments, the method includes generating a diagnosis output based on the disease indicator.

In one aspect, a method of training a model to diagnose a subject with respect to a pancreatic cancer (PC) disease state is described in accordance with various embodiments. In various embodiments, the method includes receiving quantification data (which may also be referred to as peptide structure data) for a panel of peptide structures for a plurality of subjects. In various embodiments, the plurality of subjects includes a first portion diagnosed with a negative diagnosis of a PC disease state and a second portion diagnosed with a positive diagnosis of the PC disease state. In various embodiments, the quantification data comprises a plurality of peptide structure profiles for the plurality of subjects. In various embodiments, the method includes training a machine learning model using the quantification data to diagnose a biological sample with respect to the PC disease state using a group of peptide structures associated with the PC disease state. In various embodiments, the group of peptide structures is identified in Table 16. In various embodiments, the group of peptide structures is listed in Table 16 with respect to relative significance to diagnosing the biological sample.

In one aspect, a method of monitoring a subject for a pancreatic cancer (PC) disease state is described in accordance with various embodiments. In various embodiments, the method includes receiving first peptide structure data for a first biological sample obtained from a subject at a first timepoint. In various embodiments, the method includes analyzing the first peptide structure data using a supervised machine learning model to generate a first disease indicator based on at least 1 peptide structure selected from a group of peptide structures identified in Table 16, wherein the group of peptide structures in Table 16 comprises a group of peptide structures associated with a PC disease state. In various embodiments, the method includes receiving second peptide structure data of a second biological sample obtained from the subject at a second timepoint. In various embodiments, the method includes analyzing the second peptide structure data using the supervised machine learning model to generate a second disease indicator based on the at least 1 peptide structure selected from the group of peptide structures identified in Table 16. In various embodiments, the method includes generating a diagnosis output based on the first disease indicator and the second disease indicator.

In one aspect, a composition comprising at least one of peptide structures PS-48 through PS-102 identified in Table 16 is described according to various embodiments.

In one aspect, a composition comprising at least one of peptide structures PS-49, PS-50, PS-54, PS-61, PS-63, PS-64, PS-71, PS-79, PS-81, PS-84, PS-86, PS-87, PS-90, PS-91, PS-92, PS-94, PS-95, PS-96, PS-97, PS-98, PS-99, or PS-101 identified in Table 17A is described according to various embodiments.

In one aspect, a composition comprising at least one of peptide structures PS-48, PS-52, PS-57, PS-61, PS-62, PS-63, PS-64, PS-69, PS-71, PS-72, PS-73, PS-84, PS-86, PS-88, PS-91, PS-94, PS-96, PS-100, or PS-101 identified in Table 17B is described according to various embodiments.

In one aspect, a composition comprising at least one of peptide structures PS-48, PS-52, PS-61, PS-64, PS-66, PS-68, PS-69, PS-71, PS-72, PS-73, PS-86, PS-89, PS-91, PS-94, PS-96, PS-99, or PS-101 identified in Table 17C is described according to various embodiments.

In one aspect, a composition comprising a peptide structure or a product ion is described according to various embodiments. In various embodiments, the peptide structure or the product ion comprises an amino acid sequence having at least 90% sequence identity to any one of SEQ ID NOS: 77-119, corresponding to peptide structures PS-48 through PS-102 in Table 16. In various embodiments, the product ion is selected as one from a group consisting of product ions identified in Table 18 including product ions falling within an identified m/z range.

In one aspect, a composition comprising a glycopeptide structure selected as one from a group consisting of peptide structures PS-48 through PS-102 identified in Table 16 according to various embodiments. In various embodiments, the glycopeptide structure comprises an amino acid peptide sequence identified in Table 19 as corresponding to the glycopeptide structure and a glycan structure identified in Table 21 as corresponding to the glycopeptide structure in which the glycan structure is linked to a residue of the amino acid peptide sequence at a corresponding position identified in Table 16. In various embodiments, the glycan structure has a glycan composition.

In one aspect, a composition comprising a peptide structure selected as one from a plurality of peptide structures identified in Table 16 according to various embodiments. In various embodiments, the peptide structure has a monoisotopic mass identified as corresponding to the peptide structure in Table 16. In various embodiments, the peptide structure comprises the amino acid sequence of SEQ ID NOs: 77-119 identified in Table 16 as corresponding to the peptide structure.

In one aspect, a composition comprising a peptide structure or a product ion is described according to various embodiments. In various embodiments, the peptide structure or the product ion comprises an amino acid sequence having at least 90% sequence identity to any one of SEQ ID NOS: 77-119. In various embodiments, the product ion is selected as one from a group consisting of product ions identified in Table 18 including product ions falling within an identified m/z range.

In one aspect, a composition comprising a glycopeptide structure selected as one from a group consisting of peptide structures PS-48 through PS-102 identified in Table 16 is described according to various embodiments. In various embodiments, the glycopeptide structure comprises an amino acid peptide sequence identified in Table 19 as corresponding to the glycopeptide structure. In various embodiments, a glycan structure identified in Table 21 as corresponding to the glycopeptide structure in which the glycan structure is linked to a residue of the amino acid peptide sequence at a corresponding position identified in Table 16. In various embodiments, the glycan structure has a glycan composition.

In one aspect, a composition comprising a peptide structure selected as one of PS-48 through PS-102 peptide structures identified in Table 16 is described according to various embodiments. In various embodiments, the peptide structure has a monoisotopic mass identified as corresponding to the peptide structure in Table 16. In various embodiments, the peptide structure comprises the amino acid sequence of SEQ ID NOS: 77-119 identified in Table 16 as corresponding to the peptide structure.

In one aspect, a kit comprising at least one agent for quantifying at least one peptide structure identified in Table 16 to carry out part or all of any one or more of the methods described herein.

In one aspect, a kit comprising at least one agent for quantifying at least one peptide structure identified in Table 17A, 17B, or 17C to carry out part or all of any one or more of the methods described herein.

In one aspect, a kit comprising at least one of a glycopeptide standard, a buffer, or a set of peptide sequences to carry out part or all of any one or more of the methods described herein, a peptide sequence of the set of peptide sequences identified by a corresponding one of SEQ ID NOS: 77-119, defined in Table 16 is described according to various embodiments.

In one aspect, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one aspect, a computer-program product tangibly embodied in a non-transitory machine-readable storage medium, including instructions configured to cause one or more data processors to perform part or all of any one or more of the methods described herein.

Embodiments of the disclosure include methods for quality control (QC) of samples, the method comprising: analyzing peptide structure data for each sample of a cohort using a model to generate a predicted age associated for each sample of the cohort, wherein each sample corresponds to a subject having an associated chronological age; and identifying a quality control issue associated with the chronological age for the cohort based on a correlation coefficient of the predicted age and the chronological age for each sample of the cohort. The peptide structure data may comprise a set of age-associated glycosylation biomarkers, and a set of corresponding signals associated with each of the age-associated glycosylation biomarkers, wherein the set of corresponding signals is proportional to an amount of each of the age-associated glycosylation biomarkers in the sample, wherein the model is based on the set of age-associated glycosylation biomarkers and the set of corresponding signals associated with each of the age-associated glycosylation biomarkers, and wherein the set of age-associated glycosylation biomarkers comprises at least one of the age-associated glycosylation biomarkers listed in Table 23; the method further comprising: generating the correlation coefficient based on the predicted age and the chronological age for each sample of the cohort. In some embodiments, identifying the quality control issue associated with the chronological age for the cohort is based on the correlation coefficient, wherein the correlation coefficient does not fall within a predetermined range of values. In some cases, the predetermined range of values ranges from about 0 to about 0.2. In some embodiments, the quality control issue includes an error of mislabeled samples, an error from sample preparation, a systemic measurement error, or an instrument error.

In various embodiments, methods of the disclosure comprise receiving the peptide structure data for each sample of the cohort from a mass spectrometer. In some embodiments, the correlation coefficient comprises a Pearson correlation coefficient where the predicted age is a continuous variable and the chronological age is another continuous variable. In specific embodiments, the Pearson correlation coefficient (r_{xy}) comprises an equation, the equation being

$${r}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$

$$where n = a number of the samples in the cohort ,$$

$$i = an index number for each of the samples ,$$

$${x}_{i} = a chronological age for sample i ,$$

$$\overline{x} = a mean chronological age$$

$${y}_{i} = a predicted age for sample i ,$$

$$\overline{y} = a mean predicted age .$$

In certain embodiments, the model comprises multiplying the corresponding signal associated with age-associated glycosylation biomarkers and a respective coefficient for each sample of the cohort to form a plurality of products; summing together the plurality of products to form a summation; and adding the summation and the intercept to form an output value, wherein the output value is proportional to the predicted age for the sample.

In some embodiments, the model comprises an equation, the equation being

$$O V = {\sum }_{i = 1}^{i = 20} [ ( {Signal}_{SEQ ID No : i} ) \times ( {Coefficient}_{SEQ ID No : i} ) ] + Intercept$$

$$where O V = an output value ,$$

$$i = an index number for each of the age - associated glycosylation biomarkers ,$$

$${Signal}_{SEQ ID No : i} = a corresponding signal associated with the age - associated glycosylation biomarker i , and$$

$${Coefficient}_{SEQ ID No : i} = a coefficient associated with the age - associated glycosylation biomarker i , wherein the output value is proportional to the predicted age for the sample .$$

Each sample of the cohort comes from a subject with a disease condition, the disease condition selected from the group consisting of non-small cell lung cancer, breast cancer, pancreatic cancer, colorectal cancer, and nonalcoholic steatohepatitis (NASH), in some embodiments. Each sample of the cohort may come from a subject that has either a disease condition or a healthy condition, the disease condition selected from the group consisting of non-small cell lung cancer, breast cancer, pancreatic cancer, colorectal cancer, and nonalcoholic steatohepatitis (NASH).

In particular embodiments, the at least one of the age-associated glycosylation biomarkers comprises a glycopeptide structure defined by a peptide sequence and a glycan structure linked to the peptide sequence at a linking site of the peptide sequence, as identified in Table 23, with the peptide sequence being one of SEQ ID NOS: 163-174 as defined in Tables 25A and 25B. The peptide structure data comprise at least one of a raw abundance, an adjusted raw abundance, a peptide concentration, a glycopeptide concentration, or a normalized concentration, in certain cases. In some cases, the peptide structure data comprise normalized concentration data, wherein the normalized concentration data is a function of at least one of peptide abundance data, corresponding internal standard abundance data, a spike-in concentration value, and a dilution factor. The peptide structure data may be generated using multiple reaction monitoring mass spectrometry (MRM-MS).

In particular embodiments, the method further comprises creating a sample from the biological sample; and preparing the sample using reduction, alkylation, and enzymatic digestion to form a prepared sample that includes a set of peptide structures. Any method may further comprise generating the peptide structure data from the prepared sample using multiple reaction monitoring mass spectrometry (MRM-MS), wherein each of the age-associated glycosylation biomarkers of the set comprises a precursor ion and at least one product ion in accordance with Tables 23 and 24. The set of age-associated glycosylation biomarkers comprises at least three, four, or five of the age-associated glycosylation biomarkers listed in Table 23.

Embodiments of the disclosure include methods of generating a model to predict an age of a patient, the method comprising receiving peptide structure data for each sample of a cohort, wherein each sample has a chronological age, wherein the peptide structure data for each sample of the cohort comprise a set of glycopeptide groups, wherein each glycopeptide of the glycopeptide group has a same peptide sequence, and wherein each glycopeptide of the glycopeptide group has a different attached glycan at a same specific amino acid residue; determining, via principal component analysis (PCA), one or more PCA features for each glycopeptide group of the set; performing linear regression with the PCA feature and the chronological age for each sample; and selecting a set of the one or more PCA features with statistically significant values below a threshold value. The statistically significant values for each of the PCA features may be an output of the performed linear regression. Each of the glycopeptide groups comprises one or more glycopeptides, the method further comprising training, via one or more processors, at least one machine learning model using the one or more glycopeptides for each glycopeptide group of the selected set of the one or more PCA features and the chronological age for each sample.

In some embodiments, a method may further comprise testing the at least one trained machine learning model using another cohort of samples to generate predicted age values and comparing the predicted age values with the chronological age values of the another cohort of samples to validate the at least one trained machine learning model. The trained machine learning model may comprise ElasticNet. Each sample of the cohort may come from a subject with a disease condition, the disease condition selected from the group consisting of non-small cell lung cancer, breast cancer, pancreatic cancer, colorectal cancer, and nonalcoholic steatohepatitis (NASH). Each sample of the cohort may come from a subject that has either a disease condition or a healthy condition, the disease condition selected from the group consisting of non-small cell lung cancer, breast cancer, pancreatic cancer, colorectal cancer, and nonalcoholic steatohepatitis (NASH).

Embodiments of the disclosure include methods of performing quality control for a group of subject samples, comprising assaying for, or measuring, from the group of subject samples for one or more age-related peptide structures identified in Table 23; and comparing the presence or quantity of said one or more age-related peptide structures from the group of subject samples to a reference set of one or more of the age-related peptide structures. The one or more age-related peptide structures in the reference set may be 1, 2, 3, 4, 5, 6, 7, 8, 9, 10, 11, 12, 13, 14, 15, 16, 17, 18, 19, or all 20 of the age-related peptide structures identified in Table 23. In specific embodiments, the subjects in the group comprise healthy subjects, diseased subjects, or both. The diseased subjects may have cancer or may be at an increased risk for having cancer compared to the general population. In some embodiments, the assaying or measuring step comprises mass spectrometry.

Embodiments of the disclosure include methods of measuring for one or more age-related peptide structures from one or more subject samples, wherein the chronological age of said subject(s) is known or unknown, comprising the step of assaying the one or more subject samples for one or more peptide structures in Table 23. Embodiments of the disclosure include methods of identifying or predicting the chronological age of a subject based on one or more samples therefrom, comprising the step of assaying for, or measuring, in the sample(s) for one or more age-related peptide structures identified in Table 23. In specific embodiments, the assaying or measuring is for 1, 2, 3, 4, 5, 6, 7, 8, 9, 10, 11, 12, 13, 14, 15, 16, 17, 18, 19, or all 20 age-related peptide structures of Table 23.

Embodiments of the disclosure include methods for quality control (QC) of samples, the method comprising analyzing peptide structure data for each sample of a cohort using a model to generate a predicted sex associated for each sample of the cohort, wherein each sample corresponds to a subject having an associated annotated sex; and identifying a quality control issue associated with the annotated sex for the cohort based on an accuracy score of the predicted sex and the annotated sex for each sample of the cohort. In specific embodiments, the peptide structure data comprises a set of sex-associated glycosylation biomarkers, and a set of corresponding signals associated with each of the sex-associated glycosylation biomarkers, wherein the set of corresponding signals is proportional to an amount of each of the sex-associated glycosylation biomarkers in the sample, wherein the model is based on the set of sex-associated glycosylation biomarkers and the set of corresponding signals associated with each of the sex-associated glycosylation biomarkers, and wherein the set of sex-associated glycosylation biomarkers comprises at least one of the sex-associated glycosylation biomarkers listed in Table 28; the method further comprising generating the accuracy score based on the predicted sex and the annotated sex for each sample of the cohort. In a specific embodiment, the identifying the quality control issue associated with the annotated sex for the cohort is based on the accuracy score, wherein the accuracy score is generated by determining a number of times the predicted sex is the same as that of the sex of each sample. The method may further comprise generating a sensitivity score based on the predicted sex and the annotated sex with each sample and/or generating a specificity score based on the predicted sex and the annotated sex with each sample. In specific embodiments, the quality control issue includes an error of mislabeled samples, an error from sample preparation, a systemic measurement error, and/or an instrument error. In specific cases, the method further comprises receiving the peptide structure data for each sample of the cohort from a mass spectrometer.

In specific embodiments, each sample of the cohort comes from a subject that has a disease condition, the disease condition selected from the group consisting of non-small cell lung cancer, breast cancer, pancreatic cancer, colorectal cancer, and nonalcoholic steatohepatitis (NASH). In specific embodiments, each sample of the cohort comes from a subject that has either a disease condition or a healthy condition, the disease condition selected from the group consisting of non-small cell lung cancer, breast cancer, pancreatic cancer, colorectal cancer, and nonalcoholic steatohepatitis (NASH).

In particular embodiments, the at least one of the sex-associated glycosylation biomarkers comprises a glycopeptide structure defined by a peptide sequence and a glycan structure linked to the peptide sequence at a linking site of the peptide sequence, as identified in Table 28, with the peptide sequence being one of SEQ ID NOS: 183-196 as defined in Table 30A and 30B. The peptide structure data may comprise at least one of a raw abundance, an adjusted raw abundance, a peptide concentration, a glycopeptide concentration, or a normalized concentration. The peptide structure data may comprise normalized concentration data, wherein the normalized concentration data is a function of at least one of peptide abundance data, corresponding internal standard abundance data, a spike-in concentration value, and a dilution factor. In specific embodiments, the peptide structure data are generated using multiple reaction monitoring mass spectrometry (MRM-MS). In some embodiments, the method may further comprise creating a sample from the biological sample; and preparing the sample using reduction, alkylation, and enzymatic digestion to form a prepared sample that includes a set of peptide structures. The method in some embodiments further comprises generating the peptide structure data from the prepared sample using multiple reaction monitoring mass spectrometry (MRM-MS). In some cases, the set of sex-associated glycosylation biomarkers comprises at least three, at least four, or at least five of the sex-associated glycosylation biomarkers listed in Table 28.

Embodiments of the disclosure include methods of generating a model to predict a sex of a patient, the method comprising receiving peptide structure data for each sample of a cohort, wherein each sample has an annotated sex, wherein the peptide structure data for each sample of the cohort comprise a set of glycopeptide groups, wherein each glycopeptide of the glycopeptide group has a same peptide sequence, and wherein each glycopeptide of the glycopeptide group has a different attached glycan at a same specific amino acid residue; determining, via principal component analysis (PCA), one or more PCA features for each glycopeptide group of the set; performing linear regression with the PCA feature and the annotated sex for each sample; and selecting a set of the one or more PCA features with statistically significant values below a threshold value. In some embodiments, the statistically significant values for each of the PCA features is an output of the performed linear regression. Each of the glycopeptide groups may comprise one or more glycopeptides, the method further comprising training, via one or more processors, at least one machine learning model using the one or more glycopeptides for each glycopeptide group of the selected set of the one or more PCA features and the annotated sex for each sample. In some embodiments, the method further comprises testing the at least one trained machine learning model using another cohort of samples to generate predicted sex values and comparing the predicted sex values with the annotated sex values of the another cohort of samples to validate the at least one trained machine learning model, which may comprise ElasticNet. In specific embodiments, each sample of the cohort has a disease condition, the disease condition selected from the group consisting of non-small cell lung cancer, breast cancer, pancreatic cancer, colorectal cancer, and nonalcoholic steatohepatitis (NASH). In some embodiments, each sample of the cohort has either a disease condition or a healthy condition, the disease condition selected from the group consisting of non-small cell lung cancer, breast cancer, pancreatic cancer, colorectal cancer, and nonalcoholic steatohepatitis (NASH).

Embodiments of the disclosure include methods of performing quality control for a group of subject samples, comprising: assaying for, or measuring, from the group of subject samples for one or more sex-related peptide structures identified in Table 28; and comparing the presence or quantity of said one or more sex-related peptide structures from the group of subject samples to a reference set of one or more of the sex-related peptide structures. In specific embodiments, the threshold number is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 sex-related peptide structures. The subjects in the group may comprise healthy subjects, diseased subjects (subjects have cancer or are at an increased risk for having cancer compared to the general population), or both. The assaying or measuring step may comprise mass spectrometry.

Embodiments of the disclosure include methods of measuring for one or more sex-related peptide structures from one or more subject samples, wherein the sex of said subject(s) is known or unknown, comprising the step of assaying the one or more subject samples for one or more peptide structures in Table 28.

Embodiments of the disclosure include methods of identifying or predicting the sex of a subject based on one or more samples therefrom, comprising the step of assaying or measuring in the sample(s) for one or more sex-related peptide structures identified in Table 28. The assaying or measuring may be for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or all 42 sex-related peptide structures of Table 28.

In some embodiments, provided herein is a method of classifying a biological sample obtained from a subject with respect to a plurality of states associated with non-small cell lung cancer (NSCLC). In some embodiments, the method includes receiving peptide structure data corresponding to a set of proteins in the biological sample, inputting quantification data identified from the peptide structure data for a set of peptide structures into a machine-learning model trained to identify a disease indicator based on the quantification data. In some embodiments, the set of peptide structures includes at least one peptide structure identified from a plurality of peptide structures in Table 35. In some embodiments, the method further includes identifying, by the machine-learning model, the disease indicator; and classifying the biological sample with respect to a plurality of states associated with NSCLC based upon the identified disease indicator. In some embodiments, the at least one peptide structure comprises a glycopeptide. In some embodiments, the set of proteins comprises one or more glycoproteins.

Also provided herein is a method of detecting the presence of non-small cell lung cancer (NSCLC) in a subject. In some embodiments, the method includes receiving peptide structure data corresponding to a set of proteins in a biological sample obtained from a subject. In some embodiments, the peptide structure data includes at least one peptide structure from Table 35. In some embodiments, the method further includes inputting quantification data identified from the peptide structure data for a set of peptide structures into a machine-learning model trained to identify a disease indicator based on the quantification data, and detecting the presence of NSCLC in response to a determination that the identified disease indicator falls within a selected range associated with NSCLC. In some embodiments, the at least one peptide structure comprises a glycopeptide. In some embodiments, the set of proteins comprises one or more glycoproteins.

In some embodiments, the plurality of states includes at least one of an NSCLC state or a healthy state. In some embodiments, the machine-learning model includes a regularized regression model. In some embodiments, the regularized regression model includes a least absolute shrinkage and selection operator (LASSO) regression model.

In some embodiments, the quantification data for a peptide structure of the set of peptide structures includes at least one of an abundance, a relative abundance, a normalized abundance, or a differential abundance. In some embodiments, the quantification data for a peptide structure of the set of peptide structures includes at least one of a relative quantity, an adjusted quantity, a normalized quantity, a relative concentration, an adjusted concentration, or a normalized concentration.

In some embodiments, the quantification data is generated using a liquid chromatography-mass spectrometry (LC-MS) system. In some embodiments, the peptide structure data is generated using multiple reaction monitoring mass spectrometry (MRM-MS). In some embodiments, the machine-learning model was trained utilizing a portion of the quantification data corresponding to a set of peptide structures that is a subset of the panel of peptide structures to determine which state of the plurality of states the biological sample from the subject corresponds. In some embodiments, the biological sample comprises at least one of blood, serum, or plasma.

In some embodiments, the methods further include performing a differential expression analysis using the quantification data for the plurality of subjects.

Also provided herein is a method of treating non-small cell lung cancer (NSCLC) in a subject. In some embodiments, method includes receiving peptide structure data corresponding to a set of proteins in the biological sample obtained from a subject. In some embodiments, the peptide structure data comprises at least one peptide structure from Table 35. In some embodiments, the method further includes inputting quantification data for the at least one peptide structure into a machine-learning model trained to generate disease indicator for NSCLC based on the quantification data, identifying, by the machine-learning model, the disease indicator, and determining at least one of a plurality of treatment regimens to treat NSCLC based upon the disease indicator. In some embodiments, the method further includes administering a selected treatment regimen to the subject. In some embodiments, the set of proteins comprises one or more glycoproteins.

Also provided herein is a method of treating non-small cell lung cancer (NSCLC) in a subject. In some embodiments, method includes receiving peptide structure data corresponding to a set of proteins in the biological sample, inputting quantification data identified from the peptide structure data for a set of peptide structures into a machine-learning model trained to identify a disease indicator based on the quantification data. In some embodiments, the peptide structure data includes at least one peptide structure identified from a plurality of peptide structures in Table 35. In some embodiments, the method further includes identifying, by the machine-learning model, the disease indicator, determining a classification for NSCLC based upon the identified disease indicator, and determining at least one of a plurality of treatment regimens to treat NSCLC based upon the classification. In some embodiments, the method further includes administering a selected treatment regimen to the subject. In some embodiments, the set of proteins comprises one or more glycoproteins.

Also provided herein is a method of diagnosing an individual with non-small cell lung cancer (NSCLC). In some embodiments, the method includes detecting the presence or amount of at least one peptide structure structures from Table 35 or Table 40, inputting a quantification of the detected at least one peptide structure into a machine-learning model trained to generate a class label, determining if the class label is above or below a threshold for a classification, identifying a diagnostic classification for the individual based on whether the class label is above or below a threshold for the classification, and diagnosing the individual as having NSCLC based on the diagnostic classification.

In some embodiments, the quantification data is generated using a liquid chromatography-mass spectrometry (LC-MS) system. In some embodiments, the peptide structure data is generated using multiple reaction monitoring mass spectrometry (MRM-MS). In some embodiments, the amount of at least one peptide structure is none, or below a detection limit. In some embodiments, the NSCLC is one of early-stage or late-stage NSCLC. In some embodiments, the NSCLC is one of stage I NSCLC, stage II NSCLC, stage III NSCLC, or stage IV NSCLC. In some embodiments, the at least one peptide structure comprises three or more peptide structures identified in Table 35 or Table 40. In some embodiments, the at least one peptide structure comprises at least one peptide comprising the sequence set forth in any one of SEQ ID NOs: 224-296. In some embodiments, the individual is determined have a healthy state, wherein a healthy state comprises the absence of NSCLC.

In some embodiments, the methods further include assessing one or more risk factor or clinical indicators of NSCLC. In some embodiments, the methods further include generating a report that includes a diagnosis based on the corresponding state detected for the subject.

Also provided herein is a method of training a model to diagnose a subject with one of a plurality of states associated with non-small cell lung cancer (NSCLC). In some embodiments, the method includes receiving quantification data for a panel of peptide structures for a plurality of subjects diagnosed with the plurality of states associated with NSCLC, and training a machine-learning model to determine a state of the plurality of states a biological sample from the subject based on the quantification data.

In some embodiments, training the machine-learning model to determine the state of the plurality of states further includes training the machine-learning model to generate a class label for the state of the plurality of states. In some embodiments, training the machine-learning model to determine the state of the plurality of states comprises training and evaluating the machine-learning model based on one or more of: a first set of peptide structure coefficient from Table 39 for all stages of NSCLC; a second set of peptide structure coefficients from Table 39 for early-stage NSCLC; and a third set of peptide structure coefficients from Table 39 for late-stage NSCLC. In some embodiments, the first set of peptide structure coefficients comprise the amino acid sequence of SEQ ID NOs: 229, 231-234, 236, 239, 241, 244-245, 247-255. In some embodiments, the second set of peptide structure coefficients comprise the amino acid sequence of SEQ ID NOs: 224, 227, 233, 238, 240-242, 244, 247-248, 250-255. In some embodiments, the third set of peptide structure coefficients comprise the amino acid sequence of SEQ ID NOs: 225-226, 228-230, 233-237, 239, 241-244, 246, 254-257.

In some embodiments, the plurality of states comprises at least one of a NSCLC state or a healthy state. In some embodiments, the machine-learning model comprises a regularized regression model. In some embodiments, the regularized regression model comprises a least absolute shrinkage and selection operator (LASSO) regression model.

In some embodiments, at least one of the peptide structures comprises a glycopeptide.

In some embodiments, the at least one peptide structure comprises at least one, at least two, at least three, at least five, at least 10, at least 15, at least 20, or at least 25 different peptides comprising the sequence set forth in any one of SEQ ID NOs: 224-257.

In some embodiments, the at least one peptide structure comprises at least one, at least two, at least three, at least five, at least 10, at least 15, at least 20, or at least 25 different peptides comprising the sequence set forth in any one of SEQ ID NOs: 258-296.

In some embodiments, the at least one peptide structure comprises at least one, at least two, at least three, at least five, at least 10, or at least 15 different peptides comprising the sequence set forth in any one of SEQ ID NOs: 229, 231-234, 239, 241, 244-245, 247-255.

In some embodiments, the at least one peptide structure comprises at least one, at least two, at least three, at least five, at least 10, at least 15, at least 20, or at least 25 different peptides comprising the sequence set forth in any one of SEQ ID NOs: 224-257. In some embodiments, the at least one peptide structure comprises at least one, at least two, at least three, at least five, at least 10, at least 15, at least 20, or at least 25 different peptides comprising the sequence set forth in any one of SEQ ID NOs: 258-296. In some embodiments, the at least one peptide structure comprises at least one, at least two, at least three, at least five, at least 10, or at least 15 different peptides comprising the sequence set forth in any one of SEQ ID NOs: 229, 231-234, 239, 241, 244-245, 247-255.

In some embodiments, the at least one peptide structure comprises a peptide sequence and a glycan structure, wherein the glycan structure is attached to a linking site position in the peptide sequence in accordance with Table 35. In some embodiments, the glycan structure of the peptide sequence corresponds to a glycan structure GL number in accordance with Table 35, wherein the glycan structure comprises a symbol structure in accordance with the glycan structure GL number according to Table 35, Table 36A, and Table 36B. In some embodiments, the glycan structure of the peptide sequence corresponds to a glycan structure GL number in accordance with Table 35, wherein the glycan structure comprises a composition in accordance with the glycan structure GL number, Table 35, Table 36A, and Table 36B. In some embodiments, a rightmost N-acetylgalactosamine (open square) of the glycan structure in Table 36A is attached to a linking site position in the peptide sequence in accordance with Table 35. In some embodiments, a bottommost N-acetylglucosamine (dark square) of the glycan structure in Table 36B is attached to a linking site position in the peptide sequence in accordance with Table 35.

In some embodiments, provided herein is a composition comprising one or more peptide structures from Table 35. In some embodiments, the at least one peptide structure comprises a peptide sequence and a glycan structure, wherein the glycan structure is attached to a linking site position in the peptide sequence in accordance with Table 35. In some embodiments, the glycan structure of the peptide sequence corresponds to a glycan structure GL number in accordance with Table 35, wherein the glycan structure comprises a symbol structure in accordance with the glycan structure GL number according to Table 35, Table 36A, and Table 36B. In some embodiments, the glycan structure of the peptide sequence corresponds to a glycan structure GL number in accordance with Table 35, wherein the glycan structure comprises a composition in accordance with the glycan structure GL number, Table 35, Table 36A, and Table 36B. In some embodiments, a rightmost N-acetylgalactosamine (GalNAc) of the glycan structure in Table 36A is attached to a linking site position in the peptide sequence in accordance with Table 35. In some embodiments, a bottommost N-acetylglucosamine (GlcNAc) of the glycan structure in Table 36B is attached to a linking site position in the peptide sequence in accordance with Table 35.

In some embodiments, provided herein is a composition comprising one or more peptides comprising the sequence set forth in SEQ ID NOs: 224-296. In some embodiments, the one or more peptides comprise one or more glycopeptides.

In an embodiment, a method for diagnosing a subject with respect to an ovarian cancer disease state is described. The method includes receiving peptide structure data corresponding to a biological sample obtained from the subject. The peptide structure data can be analyzed using a supervised machine learning model to generate a disease indicator that indicates whether the biological sample evidences the ovarian cancer disease state of having early stage or late stage ovarian cancer based on at least one peptide structures selected from one of a group of peptide structures identified in Tables 43B, 43C, or 43D. A diagnosis output can be generated based on the disease indicator. The disease indicator can include a score.

The method of generating the diagnosis output can include determining that the score falls above a selected threshold and generating the diagnosis output based on the score falling above the selected threshold, wherein the diagnosis output includes a classification of late stage ovarian cancer disease state. The method of generating the diagnosis output can include determining that the score falls below a selected threshold and generating the diagnosis output based on the score falling below the selected threshold, wherein the diagnosis output includes a classification of early stage ovarian cancer disease state. The score may include a probability score and the selected threshold is 0.5. Alternatively, the selected threshold may fall within a range between 0.30 and 0.65. In an embodiment, the analyzing the peptide structure data can include analyzing the peptide structure data using a binary classification model. The peptide structure of the at least one peptide structures can include a glycopeptide structure defined by a peptide sequence and a glycan structure linked to the peptide sequence at a linking site of the peptide sequence, as identified in Table 43D, with the peptide sequence being one of SEQ ID NOS: 500-549 in Table 43D as defined in Table 45. The peptide structure of the at least one peptide structures can include a glycopeptide structure defined by a peptide sequence and a glycan structure linked to the peptide sequence at a linking site of the peptide sequence, as identified in Table 43B, with the peptide sequence being one of SEQ ID NOS: 310, 314, 429, 430, 434, 436, 439, 442, 451, 453, 457, 465, 466, 467, 468, 469, 470, 471, 472, 473, and 474 in Table 43B as defined in Table 45.

In another embodiment, the method can include training the supervised machine learning model using training data, wherein the training data comprises a plurality of peptide structure profiles for a plurality of subjects and a plurality of subject diagnoses for the plurality of subjects, wherein the plurality of subject diagnoses includes a diagnosis for any subject of the plurality of subjects determined to have early stage or late stage ovarian cancer.

In another embodiment, the method can include performing a differential expression analysis using initial training data to compare a first portion of the plurality of subjects diagnosed with the classification of early stage ovarian cancer disease state versus a second portion of the plurality of subjects diagnosed with the classification of late stage ovarian cancer disease state; identifying a training group of peptide structures based on the differential expression analysis for use as prognostic markers for the ovarian cancer disease state; and forming the training data based on the training group of peptide structures identified. The training of the supervised machine learning model can include reducing the training group of peptide structures to a final group of peptide structures identified in Tables 43B, 43C, or 43D.

In an embodiment, each peptide structure profile of the plurality of peptide structure profiles can include a feature selected from one of a relative abundance and a concentration for a corresponding peptide structure. The plurality of peptide structure profiles can include a first peptide structure profile with a relative abundance for a corresponding peptide structure and a second peptide structure profile with a concentration for the corresponding peptide structure. The supervised machine learning model can include a logistic regression model.

In an embodiment, the first group of peptide structures in Tables 43B, 43C, or 43D is used to distinguish between the ovarian cancer disease state being late stage or early stage. The quantification data for a peptide structure of the set of peptide structures can include at least one of an abundance, a relative abundance, a normalized abundance, a relative quantity, an adjusted quantity, a normalized quantity, a relative concentration, an adjusted concentration, or a normalized concentration.

In an embodiment, the peptide structure data can be generated using multiple reaction monitoring mass spectrometry (MRM-MS), wherein the using of the MRM-MS includes ionizing one or more glycopeptides to form ionized glycopeptides; filtering the ionized glycopeptides with a mass filter to form filtered glycopeptides; fragmenting the filtered glycopeptides in a collision chamber into product ions; and detecting the product ions.

In an embodiment, the method can include preparing a sample of the biological sample using reduction, alkylation, and enzymatic digestion to form a prepared sample that includes a set of peptide structures.

In an embodiment, the method of classifying early and late stage ovarian cancer can be implemented after the subject has already been diagnosed as having ovarian cancer. The subject can be initially diagnosed for having ovarian cancer using one or more biomarkers in Tables 41, 42, 43A, 43B, 43C, or 43D.

In an embodiment, the generating the diagnosis output can include generating a report identifying that the biological sample evidences the early stage or late stage ovarian cancer disease state.

In an embodiment, the generating a treatment output can be generated based on at least one of the diagnosis output or the disease indicator. The treatment output can include at least one of an identification of a treatment to treat the subject or a treatment plan. The treatment can include at least one of surgery, radiation therapy, a targeted drug therapy, chemotherapy, immunotherapy, hormone therapy, or neoadjuvant therapy. In some embodiments, the group of peptide structures in Tables 43B, 43C, or 43D is listed in order of relative significance to the disease indicator.

In an embodiment, the method can further include preparing a sample of the biological sample using reduction, alkylation, and enzymatic digestion to form a prepared sample that includes a set of peptide structures. The method can further include generating the peptide structure data from the prepared sample using multiple reaction monitoring mass spectrometry (MRM-MS).

In an embodiment, a method of training a model to diagnose a subject with respect to an ovarian cancer disease state having a malignant pelvic tumor is described. The method can include receiving quantification data for a panel of peptide structures for a plurality of samples for a plurality of subjects. The plurality of subjects includes a first portion diagnosed with a classification of early stage ovarian cancer disease state and a second portion diagnosed with a classification of late stage ovarian cancer disease state. The quantification data can include a plurality of peptide structure profiles for the plurality of subjects and training a machine learning model using the quantification data to diagnose a biological sample with respect to the ovarian cancer disease state using a group of peptide structures associated with the ovarian cancer disease state, wherein the group of peptide structures is identified in Tables 43B, 43C, or 43D. The machine learning model can include a logistic regression model.

The method of training the model can further include identifying an initial plurality of peptide structure profiles, filtering the initial plurality of peptide structure profiles by a coefficient of variation to generate a plurality of peptide structure profiles for use in training the machine learning model. The filtering can be performed to exclude peptide structure profiles having the coefficient of variation at or above 20%. The training of the machine learning model can include reducing the plurality of peptide structure profiles using LASSO regression to identify a final group of peptide structures identified in Tables 43B, 43C, or 43D. The quantification data for the panel of peptide structures for the plurality of subjects diagnosed with the plurality of ovarian cancer disease states can include at least one of an abundance, a relative abundance, a normalized abundance, a relative quantity, an adjusted quantity, a normalized quantity, a relative concentration, an adjusted concentration, or a normalized concentration. The trained model can use a relative abundance for a first portion of the first group of peptide structures and a concentration for a second portion of the second group of peptide structures. Each peptide structure profile of the plurality of peptide structure profiles includes a feature selected from one of a relative abundance and a concentration for a corresponding peptide structure. The plurality of peptide structure profiles can include a first peptide structure profile with a relative abundance for a corresponding peptide structure and a second peptide structure profile with a concentration for the corresponding peptide structure.

In an embodiment, a composition can include at least one of peptide structures identified in Tables 43B, 43C, or 43D.

In an embodiment, a method for diagnosing a subject with respect to an ovarian cancer disease state is described. The method can include analyzing the peptide structure data using a supervised machine learning model to generate a disease indicator that indicates whether a biological sample evidences the ovarian cancer disease state of having early stage or late stage ovarian cancer based on a group of glycopeptide structures. The group of glycopeptide structures can include tri-antennary or tetra-antennary sialic acid moieties, wherein a portion of the glycopeptide structures of the group are fucosylated. A diagnosis is then outputted based on the disease indicator. The group of glycopeptide structures can include at least one, at least three, at least five, or at least 10 glycopeptide structure identified in Tables 43B, 43C, or 43D.

In an embodiment, the peptide structure data was generated with a mass spectrometer using the biological sample obtained from the subject.

In an embodiment, the method can further include preparing a sample of the biological sample using reduction, alkylation, and enzymatic digestion to form a prepared sample that includes a set of peptide structures. The peptide structure data can be generated from the prepared sample using multiple reaction monitoring mass spectrometry (MRM-MS). The use of the MRM-MS can include ionizing one or more glycopeptides to form ionized glycopeptides; filtering the ionized glycopeptides with a mass filter to form filtered glycopeptides; fragmenting the filtered glycopeptides in a collision chamber into product ions; and detecting the product ions.

In one or more embodiments, a system comprising one or more data processors is described according to various embodiments. In various embodiments, the system comprises a non-transitory computer readable storage medium containing instructions which, when executed on the one or more data processors, cause the one or more data processors to perform part or all of any of the methods described herein.

In one or more embodiments, a computer-program product tangibly embodied in a non-transitory machine-readable storage medium, including instructions configured to cause one or more data processors to perform part or all of any one of the methods described according to various embodiments.

In one or more embodiments, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one or more embodiments, a computer-program product tangibly embodied in a non-transitory machine-readable storage medium, including instructions configured to cause one or more data processors to perform part or all of any one or more of the methods described herein.

In various embodiments, the peptide structure data is listed in Table 43D and the detected product ion comprises a first product having a m/z value listed in Table 44C.

In some embodiments, the at least one peptide structure comprises a peptide sequence and a glycan structure, wherein the glycan structure is attached to a linking site position in the peptide sequence in accordance with one of Tables 41, 42, 43A, 43B, 43C, and 43D. In some embodiments, the glycan structure of the peptide sequence corresponds to a glycan structure GL number in accordance with Tables 41, 42, 43A, 43B, 43C, and 43D, wherein the glycan structure comprises a symbol structure in accordance with the glycan structure GL number according to Tables 41, 42, 43A, 43B, 43C, 43D, and 47. In some embodiments, the glycan structure of the peptide sequence corresponds to a glycan structure GL number in accordance with Tables 41, 42, 43A, 43B, 43C, and 43D, wherein the glycan structure comprises a composition in accordance with the glycan structure GL number, Tables 41, 42, 43A, 43B, 43C, 43D, and 47. In some embodiments, a rightmost N-acetylgalactosamine (open square) of the glycan structure in Table 47 is attached to a linking site position in the peptide sequence in accordance with Tables 43A and 4. In some embodiments, a bottommost N-acetylglucosamine (dark square) of the glycan structure in Table 47 is attached to a linking site position in the peptide sequence in accordance with Tables 41, 42, 43A, 43B, 43C, 43D, and 45.

In some embodiments, provided herein is a composition comprising one or more peptide structures from Tables 41, 42, 43A, 43B, 43C, and 43D. In some embodiments, the at least one peptide structure comprises a peptide sequence and a glycan structure, wherein the glycan structure is attached to a linking site position in the peptide sequence in accordance with Tables 41, 42, 43A, 43B, 43C, and 43D. In some embodiments, the glycan structure of the peptide sequence corresponds to a glycan structure GL number in accordance with Tables 41, 42, 43A, 43B, 43C, and 43D, wherein the glycan structure comprises a symbol structure in accordance with the glycan structure GL number according to Tables 41, 42, 43A, 43B, 43C, 43D, and 47. In some embodiments, the glycan structure of the peptide sequence corresponds to a glycan structure GL number in accordance with Tables 41, 42, 43A, 43B, 43C, and 43D, wherein the glycan structure comprises a composition in accordance with the glycan structure GL number, Tables 41, 42, 43A, 43B, 43C, 43D, and 47. In some embodiments, a rightmost N-acetylgalactosamine (GalNAc) of the glycan structure in Table 47 is attached to a linking site position in the peptide sequence in accordance with Tables 43A, 43B, 43C, 43D, and 45. In some embodiments, a bottommost N-acetylglucosamine (GlcNAc) of the glycan structure in Table 47 is attached to a linking site position in the peptide sequence in accordance with Tables 41, 42, 43A, 43B, 43C, 43D, and 45.

In regards to the various embodiments, the peptide sequence can be one of SEQ ID NOS: 504-509, 511, 513, 514, 517, 522, 523, 529, 532-536, 540, and 545.

In regards to the various embodiments, the peptide structure of the at least one peptide structures comprises a glycopeptide structure defined by a peptide sequence and a glycan structure linked to the peptide sequence at a linking site of the peptide sequence, as identified in Table 43D, with the peptide sequence being one of SEQ ID NOS: 504-509, 511, 513, 514, 517, 522, 523, 529, 532-536, 540, and 545 in Table 43D as defined in Table 45.

In one or more embodiment, a method is provided for managing a treatment for a subject diagnosed with a melanoma condition. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Tables A.1-2 or Table B.1-2. A treatment output that indicates a predicted response to the treatment for the subject is generated using the treatment score.

In one or more embodiments, a method is provided for treatment management of a subject diagnosed with a melanoma condition. The method includes receiving peptide structure data corresponding to a set of peptide structures associated with a set of glycoproteins in a biological sample obtained from the subject. A plurality of treatment scores is computed using quantification data identified from the peptide structure data for a plurality of subsets of the set of peptide structures. Each treatment score of the plurality of treatment scores corresponds to a different treatment of a plurality of treatments; wherein each subset of the plurality of subsets includes at least one peptide structure identified from a plurality of peptide structures listed in Tables A.1-2 or Table B.1-2. A comparison analysis of the plurality of treatment scores is performed. A treatment output is generated based on the comparison analysis. The treatment output includes a recommended treatment plan for treating the subject.

In one or more embodiments, a method is provided for treatment management of a subject diagnosed with a melanoma condition. The method includes receiving peptide structure data corresponding to a set of peptide structures associated with a set of glycoproteins in a biological sample obtained from the subject. A first treatment score is computed for a first treatment of pembrolizumab using first quantification data identified from the peptide structure data for a first subset of the set of peptide structures. The first subset includes at least one peptide structure identified from a plurality of peptide structures listed in Tables A.1-2. A second treatment score is computed for a second treatment comprised of nivolumab and ipilimumab using second quantification data identified from the peptide structure data for a second subset of the set of peptide structures. The second subset includes at least one peptide structure identified from a plurality of peptide structures listed in Table B.1-2. A comparison analysis of the first treatment score and the second treatment score is performed. A treatment output is generated based on the comparison analysis. The treatment output identifies one of the first treatment and the second treatment as a recommended treatment for the subject.

In one or more embodiments, a method is provided for treating a subject diagnosed with a melanoma condition. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Tables A.1-2 or Table B.1. A treatment output that indicates a predicted response to a treatment for the subject is generated using the treatment score. The treatment is administered to the patient in response to the predicted response including a positive response classification. The step of administering comprises at least one of intravenous or oral administration of the recommended treatment or a derivative thereof at a therapeutic dosage. The treatment is selected as one from a group consisting of: a first treatment of pembrolizumab for which the therapeutic dosage of at least one of 200 mg every three weeks, 2 mg/kg every three weeks is administered, or 400 mg every 6 weeks; and a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered.

In one or more embodiments, a method is provided for managing a treatment for a subject diagnosed with a melanoma condition. The method includes receiving sample data for a sample population. The sample data characterizes responses of a plurality of sample subjects diagnosed with the melanoma condition to the treatment and includes sample peptide structure data for a collection of peptide structures for each subject of the plurality of sample subjects. The sample data is grouped based on the responses of the plurality of sample subjects into a first group corresponding to a first response classification and a second group corresponding to a second response classification. A differential abundance analysis is performed using the sample data to compare the first group of the sample data corresponding to the first response classification and the second group of the sample data corresponding to the second response classification to identify a set of peptide structures from the collection of peptide structures. The set of peptide structures comprises a selected N most differentiating peptide structures between the first response classification and the second response classification. Peptide structure data corresponding to a set of glycoproteins in a biological sample obtained from the subject is received. A treatment score is computed for the treatment using quantification data identified from the peptide structure data for the set of peptide structures. A treatment output that indicates a predicted response to the treatment for the subject is generated using the treatment score.

In one or more embodiments, a method of treating melanoma in a subject is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Tables A.1-2 or Table B.1. A treatment output is computed using the treatment score. A pembrolizumab treatment is administered to the subject if the treatment output includes at least one of a positive response classification for the pembrolizumab treatment or an identification of the pembrolizumab treatment as a recommended treatment.

In one or more embodiments, a method of treating melanoma in a subject is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Tables A.1-2 or Table B.1. A treatment output is computed using the treatment score. A combination treatment comprising a combination of nivolumab and ipilimumab is administered to the subject if the treatment output includes at least one of a positive response classification for the combination treatment or an identification of the combination treatment as a recommended treatment.

In one or more embodiments, a method of identifying patients with melanoma for treatment with a pembrolizumab treatment is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Tables A.1-2 or Table B.1. A treatment output is generated using the treatment score. The patient is treated with the pembrolizumab treatment if the treatment output includes at least one of a positive response classification for the pembrolizumab treatment or an identification of the pembrolizumab treatment as a recommended treatment.

In one or more embodiments, a method of identifying patients with melanoma for treatment with a combination treatment comprising nivolumab and ipilimumab is provided. The method includes receiving peptide structure data corresponding to a set of glycoproteins in a biological sample obtained from the subject. A treatment score is computed using quantification data identified from the peptide structure data for a set of peptide structures. The set of peptide structures includes at least one peptide structure identified from a plurality of peptide structures listed in Tables A.1-2 or Table B.1. A treatment output is generated using the treatment score. The patient is treated with the combination treatment if the treatment output includes at least one of a positive response classification for the combination treatment or an identification of the combination treatment as a recommended treatment.

In one or more embodiments, a method is provided for analyzing a set of peptide structures in a sample from a patient. The method includes (a) obtaining the sample from the patient; (b) preparing the sample to form a prepared sample comprising a set of peptide structures; (c) inputting the prepared sample into a reaction monitoring mass spectrometry system to detect a set of product ions associated with each peptide structure of the set of peptide structures; and (d) generating quantification data for the set of product ions using the reaction monitoring mass spectrometry system. The set of peptide structures includes at least one peptide structure selected from peptide structures PS-330 to PS-367 identified in Table 60A. The set of peptide structures includes a peptide structure that is characterized as having: (i) a precursor ion with a mass-charge (m/z) ratio within 1.5 of the m/z ratio listed for the precursor ion in Table 60A as corresponding to the peptide structure; and (ii) a product ion having an m/z ratio within ±1.0 of the m/z ratio listed for the first product ion in Table 60A as corresponding to the peptide structure.

In one or more embodiments, a composition is provided, the composition comprising a peptide structure or a product ion, wherein: the peptide structure or product ion comprises the amino acid sequence having at least 90% sequence identity to any one of SEQ ID NOS: 570-595, corresponding to peptide structures PS-330 to PS-367 in Table 55; and the product ion is selected as one from a group consisting of product ions identified in Table 60A including product ions falling within an identified m/z range.

In one or more embodiments, a composition is provided, the composition comprising a glycopeptide structure selected as one from a group consisting of peptide structures PS-330 to PS-367 identified in Table 60A. The glycopeptide structure comprises: an amino acid peptide sequence identified in Table 7 as corresponding to the glycopeptide structure; and a glycan structure identified in Table 55 as corresponding to the glycopeptide structure in which the glycan structure is linked to a residue of the amino acid peptide sequence at a corresponding position identified in Table 55. The glycan structure has a glycan composition.

In one or more embodiments, a composition is provided, the composition comprising a peptide structure selected as one from a plurality of peptide structures identified in Table 55. The peptide structure has a monoisotopic mass identified as corresponding to the peptide structure in Table 55. The peptide structure comprises the amino acid sequence of SEQ ID NOS: 570-595 identified in Table 55 as corresponding to the peptide structure.

In one or more embodiments, a kit is provided, the kit comprising at least one agent for quantifying at least one peptide structure identified in Tables A.1-2 or Table B.lto carry out at least a portion of any one of the methods disclosed herein.

In one or more embodiments, a kit is provided, the kit comprising at least one of a glycopeptide standard, a buffer, or a set of peptide sequences to carry out at least a portion of any one of the methods disclosed herein, a peptide sequence of the set of peptide sequences identified by a corresponding one of SEQ ID NOS: 570-595, defined in Table 55.

Provided herein are methods, devices, and kits for identifying glycoproteomic biomarkers and signatures for diagnosis of a disease or a condition, such as cancer, progression of the disease or condition, and response of the disease or condition to a treatment, such as treatment with immune checkpoint blockade for cancer.

Provided herein are methods for identifying one or more glycopeptide biomarkers predictive of a disease or a condition in a subject, the method comprising: (a) obtaining from a subject a first sample at a first timepoint and a second sample at a second timepoint, wherein the first sample and the second sample comprise a glycoprotein; (b) fragmenting the glycoprotein in the first sample or the second sample into one or more glycopeptides, wherein the one or more glycopeptides comprise one or more amino acid sequences selected from a group consisting of SEQ ID NO: 673-703, 731-779, and 570-595, and combinations thereof, (c) determining an amount of the one or more glycopeptides using multiple reaction monitoring mass spectrometry (MRM-MS); (d) associating the amount of the one or more glycopeptides with the first timepoint or the second timepoint, wherein the subject has a change in a disease or a condition from the first timepoint to the second timepoint; and (e) identifying as glycopeptide biomarkers the glycopeptide where the amount of the one or more glycopeptides changed from the first timepoint to the second timepoint.

Provided herein are methods for identifying one or more glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) assessing, by the computer, the predictive model using a cross-validation with n-1 subjects to generate an outcome score for a holdout subject; (d) iterating, by the computer, step (c) for each of n subjects as the holdout subject to generate an outcome score for each subject; (e) dichotomizing, by the computer, the outcome scores for each subject at a cutoff outcome score as below or above the cutoff outcome score; (f) analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide; (g) identifying, by the computer, the glycopeptide having the interaction p-value ≤0.05 as a glycopeptide biomarker for predicting the disease or the condition. In some embodiments, the cross-validation is leave-one-out cross-validation (LOOCV). In some embodiments, the cutoff outcome score was determined to optimize Harrell's C-index. In some embodiments, the interaction p-value is less than or equal to 0.01, 0.005, or 0.001 in step (g).

Provided herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 7, 9, 12, 15, 16, 18, 20, 30, 34, 37, 44, 59, 60, 61, 62, 66, 69, 70, 75, 77, 80, and 83, and combinations thereof, (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is melanoma and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1—, PD-L1—, and CTLA-4-inhibitors. In some embodiments, the recommendation comprises continuing the treatment if the output probability indicates the treatment positively influences the outcome.

Furthermore, provided herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 826-955, and combinations thereof, (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is non-small cell lung cancer (NSCLC) and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1—, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

Provided herein are glycopeptides comprising an amino acid sequence selected from a group consisting of SEQ ID NOs: 826-955, and combinations thereof.

Described herein are kits comprising a glycopeptide standard comprising a glycopeptide comprising one or more amino acid sequences selected from a group consisting of SEQ ID NOs: 826-955, and an instruction for using the glycopeptide standard for treating cancer.

In some embodiments, fragmenting comprises protease digestion. In some embodiments, fragmenting comprises applying a mechanical force. In some embodiments, the amount of one or more glycopeptides measures multiple reaction monitoring (MRM) transitions. In some embodiments, the method comprises further generating a panel of glycopeptide biomarkers comprising one or more of the glycopeptide biomarkers identified in step (e). In some embodiments, the cross-validation is leave-one-out cross-validation (LOOCV). In some embodiments, the cutoff outcome score was determined to optimize Harrell's C-index. In some embodiments, the interaction p-value is less than or equal to 0.01, 0.005, or 0.001 in step (g). In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

In one or more embodiments, a system is provided that includes one or more data processors and a non-transitory computer readable storage medium containing instructions which, when executed on the one or more data processors, cause the one or more data processors to perform part or all of one or more methods disclosed herein.

In one or more embodiments, a computer-program product is provided that is tangibly embodied in a non-transitory machine-readable storage medium and that includes instructions configured to cause one or more data processors to perform part or all of one or more methods disclosed herein.

Provided herein are methods for classifying subject as likely to respond to pembrolizumab therapy or not likely to respond to pembrolizumab therapy based upon detection of peptides and/or glycopeptides provided herein. Also provided herein are method of treating subjects comprising detecting one or more peptides or glycopeptides provided herein and providing a treatment recommendation (such as to treat with a pembrolizumab therapy or to treat with an alternative therapy.)

In one or more embodiments, a method is provided for managing a treatment for a subject diagnosed with a melanoma condition. The method includes receiving peptide structure data corresponding to a set of glycoproteins in a biological sample obtained from the subject. A treatment score can be computed using quantification data identified from the peptide structure data for a set of peptide structures, wherein the set of peptide structures includes at least one peptide structure identified from a plurality of peptide structures listed in Table 70. A treatment output can be generated that indicates a predicted response to a treatment for the subject using the treatment score wherein the biological sample was obtained from the subject after the subject has received the treatment.

In one or more embodiments, the generating of the treatment output includes generating the predicted response to the treatment based on whether the treatment score is above a selected threshold. In one or more embodiments, the selected threshold can be 0.5.

In one or more embodiments, the generating the predicted response includes identifying a first predicted response classification for the subject when the treatment score is above 0.5 and identifying a second predicted response classification for the subject when the treatment score is not above 0.5.

In one or more embodiments, the first predicted response classification is sustained control and wherein the second predicted response classification is early disruption.

In one or more embodiments, the treatment outcome includes a recommendation to modify a treatment plan for the subject.

In one or more embodiments, the recommendation for modifying the treatment plan includes at least one of selecting a different treatment for the subject, altering a dosage for the treatment, or combining the treatment with at least one other treatment.

In one or more embodiments, the computing the treatment score includes computing a proportion of the set of peptide structures having a selected abundance greater than a reference abundance.

In one or more embodiments, the reference abundance for a peptide structure of the set of peptide structures is a median of a plurality of abundances for the peptide structure across a sample population and wherein the selected abundance for a glycopeptide structure of the set of peptide structures is a relative abundance and the selected abundance for an aglycosylated peptide structure of the set of peptide structures is an absolute abundance.

In one or more embodiments, the method further includes identifying the set of peptide structures using sample data and a statistical algorithm that identifies a relative significance for each peptide structure of a collection of peptide structures corresponding to the sample data. The statistical algorithm can include a Wilcoxon rank-sum test.

In one or more embodiments, the identifying the set of peptide structures includes performing a differential abundance analysis using the sample data to compare a first portion of the sample data corresponding to a first response classification for the treatment and a second portion of the sample data corresponding to a second response classification for the treatment to identify a selected N most differentiating peptide structures between the first response classification and the second response classification.

In one or more embodiments, the selected N most differentiating peptide structures is 20 peptide structures.

In one or more embodiments, the first response classification is sustained control which indicates an absence of disruption events during a sustained period of time after treatment administration. The second response classification is early disruption which indicates a presence of at least one disruption event during an initial period of time after treatment. The sustained period of time is longer than the initial period of time.

In one or more embodiments, the sustained period of time is 12 months and the initial period of time is 6 months.

In one or more embodiments, the at least one peptide structure includes a glycopeptide structure defined by a peptide sequence and a glycan structure linked to the peptide sequence at a linking site of the peptide sequence, as identified in Tables 70 and 76, with the peptide sequence being one of SEQ ID NOS: 826-955.

In one or more embodiments, the quantification data for a peptide structure of the set of peptide structures comprises at least one of an adjusted abundance, a relative abundance, an absolute abundance, a normalized abundance, a relative quantity, an adjusted quantity, a normalized quantity, a relative concentration, an adjusted concentration, or a normalized concentration.

In one or more embodiments, the peptide structure data is generated using multiple reaction monitoring mass spectrometry (MRM-MS).

In one or more embodiments, the method further includes creating a sample from the biological sample; and preparing the sample using reduction, alkylation, and enzymatic digestion to form a prepared sample that includes a set of peptide structures.

In one or more embodiments, the method further includes generating the peptide structure data from the prepared sample using multiple reaction monitoring mass spectrometry (MRM-MS).

In one or more embodiments, the treatment output includes at least one of a design for the treatment or a therapeutic dosage for the treatment.

In one or more embodiments, the method further includes sending the treatment output to a remote system.

In one or more embodiments, the method further includes administering a therapeutic dosage of the treatment based on the predicted response being a predicted response classification that indicates the treatment will be successful.

In one or more embodiments, the method further includes administering a therapeutic dosage of the treatment based on the predicted response being sustained control.

In one or more embodiments, the predicted response to the treatment is the same for the subject if the biological sample was obtained from the subject either before or after the subject has received the treatment.

In one or more embodiments, the biological sample was obtained from the subject about 6 weeks to about 6 months after the subject has received the treatment.

In one or more embodiments, the treatment is pembrolizumab or a combination of nivolumab and ipilimumab.

In one or more embodiments, a method for treating a subject diagnosed with a melanoma condition. It includes receiving peptide structure data corresponding to a set of glycoproteins in a biological sample obtained from the subject, wherein the biological sample was obtained from the subject before the subject has received a treatment. A treatment score was computed using quantification data identified from the peptide structure data for a set of peptide structures, wherein the set of peptide structures includes at least one peptide structure identified from a plurality of peptide structures listed in Table 70. A treatment output was generated that indicates a predicted response to the treatment for the subject using the treatment score. The treatment was administered to the subject in response to the predicted response includes a positive response classification, the step of administering comprising at least one of intravenous or oral administration of the recommended treatment or a derivative thereof at a therapeutic dosage, wherein the treatment is selected as one from a group consisting of: a first treatment of pembrolizumab for which the therapeutic dosage of at least one of 200 mg every three weeks, 2 mg/kg every three weeks is administered, or 400 mg every 6 weeks; and a second treatment comprised of nivolumab and ipilimumab for which the therapeutic dosage of either 1 mg/kg nivolumab with 3 mg/kg ipilimumab or 3 mg/kg nivolumab with 1 mg/kg ipilimumab is administered. The method further includes receiving peptide structure data corresponding to a set of glycoproteins in a biological sample obtained from the subject after the administering the treatment to the subject. Another treatment score was computed using quantification data identified from the peptide structure data for the set of peptide structures, wherein the set of peptide structures includes at least one peptide structure identified from the plurality of peptide structures listed in Table 70. Another treatment output was generated that indicates a predicted response to a treatment for the subject using the another treatment score, wherein the predicted response to the treatment for the subject using the another treatment score is the same as the predicted response to the treatment for the subject using the treatment score.

In one or more embodiments, the biological sample was obtained from the subject about 6 weeks to about 6 months after the administering the treatment to the subject.

In one or more embodiments, the at least one peptide structure includes a glycopeptide structure defined by a peptide sequence and a glycan structure linked to the peptide sequence at a linking site of the peptide sequence, as identified in Tables 70 and 76, with the peptide sequence being one of SEQ ID NOS: 826-955.

### BRIEF DESCRIPTION OF THE DRAWINGS

The present disclosure is described in conjunction with the accompanying figures:

FIG. 1 is a schematic diagram of an exemplary workflow for the detection of peptide structures associated with a disease state for use in diagnosis and/or treatment in accordance with various embodiments.

FIG. 2A is a schematic diagram of a preparation workflow in accordance with various embodiments.

FIG. 2B is a schematic diagram of data acquisition in accordance with various embodiments.

FIG. 3 is a block diagram of an analysis system in accordance with various embodiments.

FIG. 4 is a block diagram of a computer system in accordance with various embodiments.

FIG. 5 is a flowchart of a process for evaluating a biological sample obtained from a subject with respect to an FLD progression in accordance with various embodiments.

FIG. 6 is a flowchart of a process for detecting the presence of a disease state associated with an FLD progression in accordance with various embodiments.

FIGS. 7A and 7B are flowcharts of a process for training a supervised machine learning model for determining which state of a plurality of states a biological sample corresponds in accordance with various embodiments. FIG. 7A generally concerns delineation of a plurality of states based on peptide structure profiles, and FIG. 7B concerns using the resultant data to determine the presence or absence of NASH.

FIG. 8 is a flow chart of a process for classifying a biological sample as corresponding to one of a plurality of states associated with fatty liver disease (FLD) progression in accordance with various embodiments.

FIG. 9 is a flowchart of a process for treating a subject for NASH in accordance with various embodiments.

FIG. 10 provides sample distribution characteristics for controls and subject with NASH for generation of the representative models herein.

FIG. 11 provides a Classifier for NASH early (F1&F2) vs NASH late (F3&F4) utilizing within normal markers only (adjusting for relative abundance of the same glycopeptide in the flanking serum run concomitantly with the samples in which the adjusting refers to taking an average of flanking serum values).

FIG. 12 shows Multivariable Classifier for Control vs NASH-early vs NASH-late utilizing within normal markers only.

FIG. 13 is a flowchart of a process for diagnosing a subject with respect to a breast cancer (BC) disease state in accordance with one or more embodiments.

FIG. 14 is a flowchart of a process for training a model to diagnose a subject with respect to breast cancer (BC) disease state in accordance with one or more embodiments.

FIG. 15 is a flowchart of a process for monitoring a subject for a breast cancer (BC) in accordance with one or more embodiments.

FIG. 16 is a receiver operating characteristic (ROC) curve in accordance with various embodiments.

FIG. 17 is a flowchart of a process for diagnosing a subject with respect to a pancreatic cancer (PC) disease state in accordance with one or more embodiments.

FIG. 18 is a flowchart of a process for training a model to diagnose a subject with respect to pancreatic cancer (PC) disease state in accordance with one or more embodiments.

FIG. 19 is a flowchart of a process for monitoring a subject for a pancreatic cancer (PC) in accordance with one or more embodiments.

FIG. 20 is a training confusion matrix showing predictive accuracy in accordance with one or more embodiments.

FIG. 21 is a test confusion matrix showing predictive accuracy in accordance with one or more embodiments.

FIG. 22 is a table showing performance metrics for the training and testing cohorts overall and by stage in accordance with one or more embodiments.

FIG. 23 is a table showing performance metrics for the training and testing cohorts by stage in accordance with various embodiments.

FIG. 24 is a flowchart of a method for quality control (QC) of samples, in accordance with various embodiments.

FIG. 25 is a flowchart of a method of generating a model to predict an age of a patient, in accordance with various embodiments.

FIG. 26 is a flowchart of a method of performing quality control of samples, in accordance with various embodiments.

FIGS. 27A, 27B, and 27C illustrate plots of predicted age versus chronological age for healthy subjects to determine correlation coefficients of various cohorts.

FIGS. 28A, 28B, and 28C illustrate plots of predicted age versus chronological age for samples that include both disease and healthy subjects to determine correlation coefficients of various cohorts.

FIG. 29 is a flowchart of a method for quality control (QC) of samples, in accordance with various embodiments.

FIG. 30 is a flowchart of a method of generating a model to predict a sex of a patient, in accordance with various embodiments.

FIG. 31 is a flowchart of a method of performing quality control of samples, in accordance with various embodiments.

FIGS. 32A, 32B, and 32C illustrate plots of accuracy score, sensitivity score, and specificity score for training and test datasets.

FIG. 33 is a flowchart of a process for classifying a biological sample obtained from a subject with respect to a plurality of states associated with NSCLC in accordance with one or more embodiments.

FIG. 34 is a flowchart of a process for detecting the presence of one of a plurality of states associated with NSCLC in a subject in accordance with one or more embodiments.

FIG. 35 is a flowchart of a process for determining one or more of a plurality of treatment regimens for treating NSCLC in a subject in accordance with one or more embodiments.

FIG. 36 is a flowchart of a process for training a model to diagnose a subject with one of a plurality of states associated with NSCLC in accordance with one or more embodiments.

FIG. 37 is a flowchart of a process for treating NSCLC in a subject in accordance with one or more embodiments.

FIG. 38 is a flowchart of a process for diagnosing an individual with NSCLC in accordance with one or more embodiments.

FIG. 39 is shows an experimental workflow for sample preparation and analysis for either plasma or serum.

FIG. 40 is a heat map showing relative abundances of the identified peptide structures in control group and NSCLC groups at different stages of NSCLC.

FIG. 41 is a model performance plot for healthy and all NSCLC stages (1-4) samples illustrating the predictive performance of a regularized regression model (e.g., LASSO regression model) on the training data sets and test data sets.

FIG. 42 illustrates a receiver-operating-characteristic (ROC) curve/area under curve (AUC) for the regularized regression model (e.g., LASSO regression model) for a healthy control and all NSCLC stages (1-4) samples.

FIG. 43 is a model performance plot for healthy and early-stage NSCLC (stages 1-2) samples illustrating the predictive performance of regularized regression model (e.g., LASSO regression model) on the training data sets and test data sets.

FIG. 44 illustrates a receiver-operating-characteristic (ROC) curve/area under curve (AUC) for the regularized regression model (e.g., LASSO regression model) for healthy and early-stage NSCLC (stages 1-2) samples.

FIG. 45 is a model performance plot for healthy and late-stage NSCLC (stages 3-4) samples illustrating the predictive performance of regularized regression model (e.g., LASSO regression model) on the training data sets and test data sets.

FIG. 46 illustrates a receiver-operating-characteristic (ROC) curve/area under curve (AUC) for the regularized regression model (e.g., LASSO regression model) for healthy and late-stage NSCLC (stages 3-4) samples.

FIG. 47 is a flowchart of a process for diagnosing a subject with respect to an ovarian cancer disease state in accordance with one or more embodiments based on Tables 41 or 42.

FIG. 48A is a flowchart of a process for diagnosing a subject with respect to ovarian cancer disease state in accordance with one or more embodiments based on Table 43A.

FIG. 48B is a flowchart of a process for diagnosing a subject with respect to ovarian cancer disease state in accordance with one or more embodiments based on Table 43B.

FIG. 49 is a flowchart of a process for training a model to diagnose a subject with respect to an ovarian cancer disease state in accordance with one or more embodiments.

FIG. 50 is a table describing the distribution of the samples acquired in this exemplary retrospective analysis in accordance with one or more embodiments.

FIG. 51 is a plot diagram illustrating the results of a principal component analysis performed to assess the segregation between healthy, benign pelvic tumor, and EOC samples across first and second principal components in accordance with one or more embodiments.

FIG. 52 is a plot diagram illustrating the results of a principal component analysis performed to assess segregation between healthy, benign pelvic tumor, early EOC, late EOC, and missing (undocumented) samples).

FIG. 53 is an illustration of a receiver operating characteristic (ROC) diagram corresponding to the multivariable model built to predict malignancy v. benign status of pelvic tumors in accordance with one or more embodiments.

FIG. 54 is an illustration of a diagram showing the probability distributions for the various groups using the multivariable model for predicting malignancy v. benign status of pelvic tumors in accordance with one or more embodiments.

FIG. 55 is an illustration of a receiver operating characteristic (ROC) diagram corresponding to the multivariable model built to predict malignancy v. benign status of pelvic tumors in accordance with one or more embodiments.

FIG. 56 is an illustration of a diagram showing the probability distributions for the various groups using the multivariable model for predicting malignancy v. benign status of pelvic tumors in accordance with one or more embodiments.

FIGS. 57A to 57E are a plurality of charts illustrating the upregulation of fucosylated biomarkers having tri or tetra-antennary sialic acids from stages 1/2 to 3/4 of ovarian cancer and the down regulation of non-fucosylated biomarkers having tri or tetra-antennary sialic acids from stages 1/2 to 3/4 of ovarian cancer.

FIG. 58 is an illustration of a diagram showing the probability distributions for early stage v. late stage ovarian cancer using training data set and the testing data set in accordance with one or more embodiments using the biomarkers of Table 43C.

FIG. 59 is an illustration of a receiver operating characteristic (ROC) diagram corresponding to the multivariable model built to predict early stage v. late stage ovarian cancer in accordance with one or more embodiments.

FIG. 60 is a graph illustrating the fold changes for a plurality of tri- and tetra-antennary glycans glycopeptides that were either non-fucosylated or fucosylated.

FIG. 61A is a graph illustrating the fold changes for pairs of tri- and tetra-antennary glycans glycopeptides that were either non-fucosylated or fucosylated.

FIG. 61B is a graph illustrating the fold changes for triplets of tri- and tetra-antennary glycans glycopeptides that were either non-fucosylated, mono-fucosylated, or di-fucosylated. Both mono-fucosylated and di-fucosylated markers has median FC's above 1 suggesting correlation of these markers with malignant EOC.

FIG. 62 is an illustration of a diagram showing the probability distributions for early stage v. late stage ovarian cancer using training data set and the testing data set in accordance with one or more embodiments using the biomarkers of Table 43D

FIGS. 63A to 63E are graphs of the relative abundance of five distinct types of fucosylated glycopeptides in benign tumors, early stage EOC, and late stage EOC.

FIG. 64 is a flowchart of a process for managing a treatment for a subject diagnosed with a melanoma condition in accordance with one or more embodiments.

FIG. 65 is a flowchart of a process for treatment management of a subject diagnosed with a melanoma condition in accordance with various embodiments.

FIG. 66 is a flowchart of a process for treatment management of a subject diagnosed with a melanoma condition in accordance with various embodiments.

FIG. 67 is a flowchart of a process for identifying a treatment for a subject diagnosed with a melanoma condition in accordance with one or more embodiments.

FIG. 68 is a plot showing the distribution of the treatment scores generated for those patients who were treated with pembro in accordance with one or more embodiments.

FIG. 69 is a plot showing the distribution of the treatment scores generated for those patients who were treated with ipi/nivo in accordance with one or more embodiments.

FIG. 70 is a scatterplot showing the treatment scores by treatment type in accordance with one or more embodiments.

FIG. 71 is a plot showing disruption event times for patients treated with pembro by their predicted response.

FIG. 72 is a plot showing disruption event times for patients treated with ipi/nivo by their predicted response.

FIGS. 73A and 73B show progression-free survival (PFS) Kaplan-Meier curves of patients with metastatic melanoma for various glycopeptide fragments.

FIGS. 74A and 74B show progression-free survival (PFS) Kaplan-Meier curves of patients with non-small-cell lung cancer (NSCLC) for various glycopeptide fragments.

FIGS. 75-100 show overall survival (OS) Kaplan-Meier curves of patients with metastatic melanoma for various glycopeptide fragments.

FIGS. 101-139 show progression-free survival (PFS) Kaplan-Meier curves of patients with metastatic melanoma for various glycopeptide fragments.

FIGS. 140A and 140B illustrate an algorithm development pipeline for identifying non-small-cell lung cancer (NSCLC).

FIGS. 141A and 141B illustrate a multivariate classifier development for case-control studies for identifying non-small-cell lung cancer (NSCLC).

FIGS. 142A-142D illustrate scoring prediction curves for identifying non-small-cell lung cancer (NSCLC).

FIG. 143 shows phenotypic data for each of three independent cohorts of baseline samples of immune checkpoint inhibitor-therapy treated NSCLC patients. There is slight heterogeneity in sex distribution and overall survival time distribution across cohort, but nothing drastic.

FIG. 144 shows that fourteen site-specific glycopeptides are associated with overall survival in the same direction of effect across the three independent cohorts. Age- and sex-adjusted univariate Cox regression show four glycopeptides that are protective against death (HR<1), while the other ten are deleterious (HR>1).

FIG. 145 shows that a subset of glycopeptides, one example of which is shown here, demonstrate the same statistically significant direction of effect across all three independent cohorts. Using Cohort 3 as the primary cohort of interest (n=125), a cutpoint was chosen that maximizes Harrell's c-index; in other words, we find two groups of patients that have maximally different overall survival outcomes based strictly on glycopeptide expression. This standardized cutpoint was then applied to Cohorts 1 and 2 in their respective Kaplan-Meier plots for the same glycopeptide biomarker.

FIG. 146 shows the results of a LASSO-regularized classifier on 88 of 125 patients in Cohort 3 (HR=5.0, P=1.0×10\hat{ }-5) using a small subset of site-specific glycopeptides and non-glycosylated peptides, and validated the classifier on the remaining 37 patients (HR=7.2, P=3.0×10\hat{ }-4). In the unseen test set, patients who are classified as “likely to benefit” (Dawn positive) from ICI therapy have a median overall survival of 23.2 months, while those who are classified as “unlikely to benefit” (Dawn negative) have a median overall survival of nearly 5 months.

FIGS. 147A and 147B show overall survival (OS) Kaplan-Meier curves of patients with non-small-cell lung cancer (NSCLC) for various glycopeptide fragments.

FIG. 148 is a chart that shows the mean risk scores of the first prediction model as a function of time after the ICI treatment.

FIG. 149 is a chart that shows the mean risk scores of the second prediction model as a function of time after the ICI treatment.

FIG. 150 is a chart that shows the mean risk scores of the first prediction model as a function of time after the specific ICI treatment combination therapy of ipilimumab (ipi) and nivolumab (nivo).

FIG. 151 is a chart that shows the mean risk scores of the first prediction model as a function of time after the specific ICI treatment monotherapy of pembrolizumab (pembro).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases, so their analysis benefits from accuracy. Glycoproteins may also be important to differentiating between stages within disease (e.g., the stages of FLD).

Although protein glycosylation provides useful information about cancer, other diseases and stage determination of a disease analysis of protein glycosylation or the analysis of protein glycosylation may be difficult as the glycan typically cannot be traced back to the protein site of origin with currently available methodologies. Glycoprotein analysis can be challenging in general for several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

However, to understand various disease conditions and disease progressions and to diagnose certain disease states more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state. This information can be used to distinguish the disease state from other states, diagnose a subject as having or not having the disease state, determine a likelihood that a subject has the disease state, or a combination thereof. Such analysis may be useful in distinguishing between, for example, without limitation, two or more stages of a non-alcoholic steatohepatitis (NASH) state, and a non-NASH/NASH state (which may include at least one of a non-alcoholic FLD disease state, a control state, a healthy state, or a liver disease-free state). For example, such analysis may be useful in diagnosing a PC disease state for a subject (e.g., a negative diagnosis for the PC disease state or a positive diagnosis for the PC disease state). Sample collection and analysis can be collected at different time points for comparing PC disease states overtime for a subject. For example, the negative diagnosis may include a healthy state, a benign pancreatitis state (i.e. “benign” as seen throughout), and/or a control state. An example of the positive diagnosis includes the subject suffering from a form of pancreatic cancer (e.g., pancreatic adenocarcinoma). A diagnosis can also assess a malignancy status of a mass previously identified on a subject's pancreas.

Accordingly, the embodiments described herein provide various methods and systems for analyzing proteins in subjects and, in particular, glycoproteins. In various embodiments, a machine learning model is trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject having a particular disease state (e.g., stage of FLD, including NASH).

The embodiments described herein recognize that the abundance of selected peptide structures in a biological sample obtained from a subject may be used to determine the likelihood of that subject evidencing a PC disease state. A PC disease state may include any condition that can be diagnosed as cancer that occurs in the pancreas (e.g., pancreatic adenocarcinoma). Further, certain peptide structures that are associated with a PC disease state may be more relevant to that disease state than other peptide structures that are also associated with that disease state.

Analyzing the abundance of peptide sequences and glycosylated peptide sequences in a biological sample may provide a more accurate way in which to distinguish the state of progression within FLD, including the stages of NASH. This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

The description below provides exemplary implementations of the methods and systems described herein for the research, diagnosis, and/or treatment (e.g., designing, planning, and/or manufacturing of a treatment) of a disease state (e.g., a NASH state) associated with FLD. Descriptions and examples of various terms, as used herein, are provided in Section II below.

The embodiments described herein recognize that the abundance of selected peptide structures in a biological sample obtained from a subject may be used to determine the likelihood of that subject evidencing a PC disease state. A PC disease state may include any condition that can be diagnosed as cancer that occurs in the pancreas (e.g., pancreatic adenocarcinoma). Further, certain peptide structures that are associated with a PC disease state may be more relevant to that disease state than other peptide structures that are also associated with that disease state.

Analyzing the abundance of peptide sequences and glycosylated peptide sequences in a biological sample may provide a more accurate way in which to distinguish a positive PC disease state (e.g., a state including the presence of pancreatic cancer) from a negative PC disease state (e.g., healthy state, control state, an absence of pancreatic cancer, benign pancreatitis, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

The description below provides exemplary implementations of the methods and systems described herein for the research, diagnosis, and/or treatment of a PC disease state. Various examples implement the methods and systems described herein as a screening tool. Descriptions and examples of various terms, as used herein, are provided in Section II below.

In addition to the above noted challenges, glycoproteomic analysis experiments can often involve large sample cohorts with hundreds or thousands of samples where each sample has relevant associated information such as, for example, the chronological age of the subject. It is worthwhile to note that the chronological age refers to the age of a subject based on the birthday of the subject and the date of blood draw. The chronological age is in contrast to an estimated age that is calculated based on the measurement of age-related biomarkers in a sample. Often, an intake form is filled out where the subject's chronological age is inputted into a database, sample manifest or report, and/or incorporated into a label. In many cases, the samples are aliquoted, randomized, treated with various reagents and enzymes, and transferred and mapped onto an auto-sampler for testing in an instrument such as a mass spectrometer. At various steps in the process, there is an opportunity to misidentify or translocate one or more samples. In addition, a clerical error can be made in recording the chronological age of one or more subjects in a cohort. Alternatively, a mapping error can be made by a laboratory operator during any one of the processing steps and/or through the use of loading an auto-sampler. As such, there is a need to perform a process check that identifies an error in the processing of the samples. In an embodiment, an estimated age can be calculated based on the measurement of age-related biomarkers, where the estimated age can be correlated to the chronological age. For situations where the estimated age and the chronological age do not have a good correlation, an error notification can be provided.

In various embodiments, a method may include estimating age and estimating the gender of the sample where the estimated age can be correlated to the chronological age and the estimated gender can be correlated to the annotated gender of the subject. For situations where the estimated age and the chronological age do not have a good correlation or the estimated gender and the annotated gender do not have a good correlation, an error notification can be provided.

Accordingly, the embodiments described herein provide various methods and systems for quality control for analyzing glycoproteins in samples from subjects. In one or more embodiments, one or more machine learning models are trained to analyze peptide structure data and generate indicators that provides information relating to quality of the analysis, particularly peptides associated with age of the individuals from which the samples were obtained. For example, in various embodiments for quality control, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of the age-related glycosylated peptides include N-linked glycopeptides and O-linked glycopeptides.

The embodiments described herein recognize that the abundance of selected peptide structures in a biological sample obtained from a subject may be used to determine the likelihood that the processing of the samples lacks significant error. Certain peptide structures are associated with the age of individuals, including associated with a range of ages in some embodiments, and these peptide structures act as a constant reference to evaluate the precision of the glycopeptide processing methods. Analyzing the abundance of peptide sequences and glycosylated peptide sequences in a plurality of biological samples may provide a more accurate way in which to ensure that the methods of their analyses were of a suitable quality.

In addition to the above noted challenges, glycoproteomic analysis experiments can often involve large sample cohorts with hundreds or thousands of samples where each sample has relevant associated information such as, for example, the annotated sex of the subject. It is worthwhile to note that the annotated sex refers to the gender of a subject that is either male or female based on biological characteristics at birth. The annotated sex is in contrast to an estimated sex that is calculated based on the measurement of sex-related biomarkers in a sample. Often, an intake form is filled out where the subject's annotated sex is inputted into a database, sample manifest or report, and/or incorporated into a label. In many cases, the samples are aliquoted, randomized, treated with various reagents and enzymes, and transferred and mapped onto an auto-sampler for testing in an instrument such as a mass spectrometer. At various steps in the process, there is an opportunity to misidentify or translocate one or more samples. In addition, a clerical error can be made in recording the annotated sex of one or more subjects in a cohort. Alternatively, a mapping error can be made by a laboratory operator during any one of the processing steps and/or through the use of loading an auto-sampler. As such, there is a need to perform a process check that identifies an error in the processing of the samples. In an embodiment, an estimated sex can be calculated based on the measurement of sex-related biomarkers, where the estimated sex can be correlated to the annotated sex. For situations where the estimated sex and the annotated sex do not have a good correlation, an error notification can be provided.

In various embodiments, a method may include estimating age and estimating the gender of the sample where the estimated age can be correlated to the chronological age and the estimated gender can be correlated to the annotated gender of the subject. For situations where the estimated age and the chronological age do not have a good correlation or the estimated gender and the annotated gender do not have a good correlation, an error notification can be provided. To ensure these processes retain accuracy for analysis of the appropriate peptides, an internal standard would be useful, in various embodiments.

Accordingly, the embodiments described herein provide various methods and systems for quality control for analyzing glycoproteins in samples from subjects. In one or more embodiments, one or more machine learning models are trained to analyze peptide structure data and generate indicators that provides information relating to quality of the analysis, particularly peptides associated with sex of the individuals from which the samples were obtained. For example, in various embodiments for quality control, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of the sex-related glycosylated peptides include N-linked glycopeptides and O-linked glycopeptides.

The embodiments described herein recognize that the abundance of selected peptide structures in a biological sample obtained from a subject may be used to determine the likelihood that the processing of the samples lacks significant error. Certain peptide structures are associated with the sex of individuals, and these peptide structures act as a constant reference to evaluate the precision of the glycopeptide processing methods. Analyzing the abundance of peptide sequences and glycosylated peptide sequences in a plurality of biological samples may provide a more accurate way in which to ensure that the methods of their analyses were of a suitable quality.

Provided herein are methods useful for diagnosing NSCLC based upon one or more biomarkers. In some embodiments, the diagnosis is based upon the presence, absence, and/or amount of one or more peptide structures comprising a sequence set forth in SEQ ID NOs: 224-296 along with the associated glycan set forth in Table 35. In some embodiments, a machine-learning model is used to classify the sample with respect to a state associated with NSCLC, such as NSCLC or a healthy state.

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and to diagnose certain diseases, such as ovarian cancer, more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state (e.g., an ovarian cancer disease state). This information can be used to distinguish the disease state from other states, diagnose a subject as having or not having the disease state, determine a likelihood that a subject has the disease state, determine whether a subject has one of early stage (stages 1 and 2) or late stage (stages 3 and 4) EOC, or a combination thereof. For example, such analysis may be useful in diagnosing an ovarian cancer disease state for a subject (e.g., a negative diagnosis for the ovarian cancer disease state or a positive diagnosis for the ovarian cancer disease state). Sample collection and analysis can be collected at different time points for comparing ovarian cancer disease states over time for a subject. For example, the negative diagnosis may include a healthy state or a benign tumor state (i.e., “benign” as seen throughout). An example of the positive diagnosis includes the subject suffering from a form of ovarian cancer (e.g., epithelial ovarian cancer (EOC)). A diagnosis can also assess a malignancy status of a previously identified pelvic (or adnexal) tumor (or mass).

Accordingly, the embodiments described herein provide various methods and systems for analyzing proteins in subjects and, in particular, glycoproteins. In one or more embodiments, a machine learning model is trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject evidencing an ovarian cancer disease state. An ovarian cancer disease state may include any condition that can be diagnosed as cancer that occurs in in the ovaries. Many malignant pelvic tumors are ovarian cancer. Certain peptide structures that are associated with an ovarian cancer disease state may be more relevant to that disease state than other peptide structures that are also associated with that disease state.

Analyzing the abundance of peptide sequences and glycosylated peptide sequences in a biological sample may provide a more accurate way in which to distinguish a positive ovarian cancer disease state (e.g., a state including the presence of ovarian cancer) from a negative ovarian cancer disease state (e.g., healthy state, a benign tumor state, an absence of ovarian cancer, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

In many instances, ovarian cancer treated with surgical resection will reoccur due to the metastasis. Thus, there is a need for tests that can diagnose metastatic ovarian cancer and monitor the progression of the disease (e.g., assessing the state of early vs late stage ovarian cancer). Such a test may be based on either ELISA or mass spectrometry.

For reference, in stage 1, the cancer is confined to the ovaries and hasn't spread to the abdomen, pelvis or lymph nodes, nor to distant sites. In stage 2, the cancer has spread from one or both ovaries to other areas of the pelvis. However, the cancer hasn't spread to nearby lymph nodes or distant sites. Stages 1 and 2 are considered early stage. In stage 3, the cancer has spread to nearby lymph nodes and/or other parts of the abdomen, but it hasn't spread to distant sites. In stage 4, the cancer has spread beyond the abdomen. Stages 3 and 4 are considered late stage.

A particular type of glycopeptides having fucosylation was found through mass spectrometry measurements to be associated with metastatic ovarian cancer. In addition, this type of glycopeptide had tri- and tetra-antennary N-glycans on certain proteins. In an embodiment, various proteins such as AGP1, AGP2, APOC3, FETUA, HPT, CLUS, A2MG, TRFE, VTNC, IGJ, and CFAH can be captured on an ELISA plate from patient samples followed by a lectin based detection (four lectins: LCA, AAL, PHA-E, PHA-L).

Mass spectrometry can be used to analyze serum for various glycoproteins and/or glycopeptides to differentiate between benign and malignant adnexal masses. Through analyzing the clinical mass spectrometry data, a distinct signature was found with the circulating N-glycoproteins that allows a differentiation between late stage (metastatic disease of stage III/IV) and early stage (stage I/II) epithelial ovarian cancer (EOC). Using Qiagen's Ingenuity Pathway Analysis package on this data, it was predicted that the signature markers are downstream of cytokine signaling. The markers also suggest the presence of the sialyl Lewis X (sLex) epitope on N-glycans of certain liver-derived circulatory glycoproteins. Given these findings suggesting the presence of sLex epitopes in circulation, it was investigated whether the outer-arm fucosylation was upregulated on the tumor itself. Bulk RNASeq data showed the outer-arm fucosyltransferases FUT3, FUT4, and FUT9 were found to be upregulated in late stage EOC. The core fucosyltransferase FUT8 on the other hand was unchanged between early and late stage EOC. A blood-based test would be useful for staging/treatment recommendations and to preempt recurrence and metastatic transformation of epithelial ovarian cancer.

Further, the methods, systems, and compositions provided by the embodiments described herein may enable an earlier and more accurate diagnosis of ovarian cancer in a subject as compared to currently available diagnostic modalities (e.g., imaging, biochemical tests) used for determining whether surgical intervention is indicated. For example, various currently available non-invasive tests to distinguish between benign and malignant pelvic tumors rely on detection of the biomarker cancer antigen 125 (CA125). But this biomarker is limited by poor sensitivity and specificity. In fact, serum CA125 is not elevated in over 20% of ovarian carcinomas and is elevated in a variety of other malignant and non-malignant conditions. While various other tests incorporate other protein biomarkers in addition to CA125, these other tests may perform less adequately than desired and may be more complex than desired. The embodiments described herein enable more reliable prediction of the malignant or benign nature of pelvic (or adnexal) tumors (or masses).

Objective response rates for immune-oncology therapy are low in malignant melanoma and non-small cell lung cancer patients. Subjects should avoid unnecessary exposure and toxicities if they will not respond to immune-oncology therapy. Thus, in some aspects, the present invention is directed to identifying subjects who are not likely to respond to immune-oncology therapy (such as treatment with pembrolizumab and/or treatment with nivolumab and ipilimumab). In some embodiments the methods provided herein increase the rate of responder to immune-oncology treatments by identifying non-responders. Another advantage of the present method is that it can be used to reduce the cost associated with immune-oncology therapy per indication by avoiding treatment of subjects that are not likely to respond to treatment.

In some aspects, the present methods employ models and other predictive methods to assess the likelihood of response of a subject to immunotherapy. In some aspects, the methods provided herein have a high sensitivity for non-responders (those that are not likely to respond to immune-oncology therapy). In some aspects, the methods provided herein have a >95%, >97%, >98, or >99% sensitivity for detection of non-responders.

Provided herein are methods for management of treatment for subjects diagnosed with melanomas. In some embodiments, the subject is diagnosed with advanced melanoma. In some embodiments, the subject is diagnosed with malignant melanoma. In some embodiments, the subject is diagnosed with metastatic melanoma. In some embodiments, the method comprises determining whether the subject is likely to respond to an immunotherapy. In some embodiments, the method comprises determining whether the subject is likely to respond to treatment with pembrolizumab. In some embodiments, the method comprises determining whether the subject is likely to respond to treatment with nivolumab and ipilimumab.

Provided herein are methods of treating melanoma in a subject comprising administering a treatment to the subject. In some embodiments, the melanoma is advanced melanoma. In some embodiments, the melanoma is malignant melanoma. In some embodiments, the melanoma is metastatic melanoma. In some embodiments, the treatment comprises administering pembrolizumab to the subject. In some embodiments, the treatment comprises administering nivolumab and ipilimumab to the subject.

In some embodiments, the method comprises determining the likelihood of response of a subject having melanoma to nivolumab plus ipilimumab as a first line therapy. In some embodiments, the method comprises determining the likelihood of response to nivolumab plus ipilimumab as a second line therapy.

In some embodiments, the method comprises determining the likelihood of response of a subject having non-small cell lung cancer to pembrolizumab as a first line therapy. In some embodiments, the method comprises determining the likelihood of response to pembrolizumab as a second line therapy.

In some embodiments, the methods provided herein comprises generating a treatment output that predicts a response to an immunoncology therapy (such as pembrolizumab or nivolumab plus ipilimumab) In some embodiments, the predicted response is likely responsive, likely nonresponsive, or indeterminate. In some embodiments, the treatment output is determined based upon the presence, absence, or amount of one or more glycopeptide set forth in Tables A.1-2 or Table B.1-2. In some embodiments, the methods provided herein predict overall survival in subjects with melanoma. In some embodiments, the methods provided herein predict progression free survival in subject with NSCLC.

### 1. Managing Treatment of Melanoma

### I. Overview

The embodiments described herein recognize that glycoproteomics is an emerging field that can be used in the overall treatment of subjects (e.g., patients) with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to treating different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and more accurately manage the treatment of such disease conditions, such as melanoma, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information that can be used to treat diseases, such as melanoma.

Melanoma is a type of cancer that develops from melanocytes, cells that product pigment. Melanoma may be treated using different types of treatment including, for example, immunotherapies. Such immunotherapies include various types of immune check point inhibitor treatments (e.g., pembrolizumab, nivolumab, ipilimumab) and cytokine therapies (e.g., interferon alpha (IFN-α) and Interleukin 2 (IL-2). Immune check point inhibitors include, for example, anti-cytotoxic T-lymphocyte-associated protein 4 (CTLA-4) monoclonal antibodies (e.g., ipilimumab, tremelimumab), toll-like receptor (TLR) agonists, cluster of differentiation 40 (CD40) agonists, anti-programmed cell death protein 1 (PD-1) (e.g., pembrolizumab, pidilizumab, and nivolumab) and programmed death-ligand 1 (PD-L1) antibodies.

Different patients may respond differently to different treatments. For example, some patients may have great success with one type of treatment while other patients may have limited or no success with that same treatment. Because melanoma is an aggressive cancer and one of the most serious cancers, subjects may not have the luxury of trying different types of treatments over time. It may be important to identify those subjects who are likely to respond to a given treatment to help avoid the burden associated with adverse events (e.g., events that disrupt a subject's progression-free survival) and to avoid the cost associated with treatment subjects who are not likely to respond to certain treatments. Previous methodologies generally focused on specific mechanisms of drug efficacy of a particular treatment. For example, such methodologies focused on tumor response rather than subject survival. But the embodiments described herein provide ways in which to predict treatment response with respect to survivability for different drugs so that a better selection of treatment may be selected for a subject at the outset.

Analyzing peptide structure expression in subjects and, in particular, glycopeptide structure abundance may help predict subject response to treatment for melanoma. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

Further, with glycoproteins, there may be too many potential proteoforms to consider. Still further, analysis of peptide structure data in the manner described by the various embodiments herein may be more conducive to accurately predicting treatment response as compared to glycomic analysis that provides little to no information about what proteins and to which amino acid residue sites various glycan structures attach.

By analyzing which peptide structures are most differentiating between different treatment response classifications of interest (e.g., sustained control and early disruption) for a given treatment and then analyzing a subject's peptide structure profile of those particular peptide structures, a clearer understanding of how that subject will respond to that treatment may be achieved.

Accordingly, the embodiments described herein provide various methods and systems for analyzing proteins in subjects and, in particular, glycoproteins. In one or more embodiments, methods and systems are provided for treatment management of a subject diagnosed with a melanoma condition. For example, the embodiments described herein provide methods and systems for receiving peptide structure data corresponding to a set of glycoproteins in a biological sample obtained from the subject; computing a treatment score using quantification data identified from the peptide structure data for a set of peptide structures, wherein the set of peptide structures includes at least one peptide structure identified from a plurality of peptide structures listed in Table 55; and generating a treatment output that indicates a predicted response to the treatment for the subject using the treatment score. The predicted response may indicate whether the subject is likely to have sustained control (e.g., no disruption events that might disrupt the subject's progression-free survival within 12 months of treatment) with the treatment or to have early disruption (e.g., one or more disruption events within the first 6 months of treatment).

The description below provides exemplary implementations of the methods and systems described herein for the research and/or treatment (e.g., designing, planning, administration, etc. of a treatment) of melanoma. Descriptions and examples of various terms, as used herein, are provided in Section II below.

Objective response rates for pembrolizumab therapy are low in non-small-cell lung cancer patients. Subjects should avoid unnecessary exposure and toxicities if they will not respond to pembrolizumab therapy. Thus, in some aspects, a method described herein is directed to identifying subjects who are not likely to respond to pembrolizumab therapy (such as treatment with pembrolizumab and/or combination therapy with pembrolizumab and chemotherapy). In some embodiments the methods provided herein increase the rate of responder to pembrolizumab therapy by identifying non-responders. Another advantage of the present method is that it can be used to reduce the cost associated with pembrolizumab therapy per indication by avoiding treatment of subjects that are not likely to respond to treatment.

In some aspects, the present methods employ models and other predictive methods to assess the likelihood of response of a subject to pembrolizumab therapy

In some embodiments, the method comprises determining the likelihood of response of a subject having non-small-cell lung cancer to pembrolizumab as a first line therapy. In some embodiments, the method comprises determining the likelihood of response to pembrolizumab as a second line therapy.

In some embodiments, the methods provided herein comprises generating a treatment output that predicts a response to pembrolizumab therapy or combination therapy with pembrolizumab and chemotherapy. In some embodiments, the predicted response is likely responsive, likely nonresponsive, or indeterminate. In some embodiments, the treatment output is determined based upon the presence, absence, or amount of one or more glycopeptide set forth in Table 78. In some embodiments, the methods provided herein predict overall survival in subjects with NSCLC.

1. Biomarkers for Determining Immuno-Oncology response—NSCLC

Provided herein are methods, devices, glycopeptides, and kits for identifying glycoproteomic biomarkers and signatures for risk of having a disease or a condition, progression of the disease or condition, and response of the disease or condition to a treatment, such as treatment with pembrolizumab for non-small-cell lung cancer. In some cases, the disease or condition may be cancer. In some cases, the progression of the disease or condition includes but is not limited to stage of cancer or size of tumor or a surrogate endpoint. Such information may be used to provide actionable recommendations for treatment to a healthcare provider, including but not limited to initiation of a new treatment, continuation of ongoing treatment, adding a new therapy, or changing the dosage and/or frequency of ongoing treatment.

Protein glycosylation is one of the abundant and most complex form of post-translational protein modification. Glycosylation profoundly can affect structure, conformation, and function of a polypeptide. The elucidation of the potential role of differential polypeptide glycosylation as biomarkers has so far been limited by the technical complexity of generating and interpreting this information. A novel, powerful platform has been established that combines ultra-high-performance liquid chromatography (LC) coupled to triple quadrupole mass spectrometry (MS) with a machine-learning and neural-network-based data processing engine that allows for high-throughput, highly scalable interrogation of the glycoproteome. The glycoproteomic biomarkers and signatures may be used to predict which cancer patients may respond to pembrolizumab therapy.

Changes in glycosylation have been described in relationship to disease states such as cancer. See, e.g., Dube, D. H.; Bertozzi, C. R. Glycans in Cancer and Inflammation —Potential for Therapeutics and Diagnostics. Nature Rev. Drug Disc. 2005, 4, 477-88, the entire contents of which are herein incorporated by reference in its entirety for all purposes. However, clinically relevant, non-invasive assays for diagnosing cancer in a patient based on glycosylation changes in a sample from that patient are still needed.

Mass spectroscopy (MS) offers sensitive and precise measurement of cancer-specific biomarkers including glycopeptides. See, for example, Ruhaak, L. R., et al., Protein-Specific Differential Glycosylation of Immunoglobulins in Serum of Ovarian Cancer Patients DOI: 10.1021/acs.jproteome.5b01071; J. Proteome Res., 2016, 15, 1002-1010 (2016); also Miyamoto, S., et al., Multiple Reaction Monitoring for the Quantitation of Serum Protein Glycosylation Profiles: Application to Ovarian Cancer, DOI: 10.1021/acs.jproteome.7b00541, J. Proteome Res. 2018, 17, 222-233 (2017), the entire contents of which are herein incorporated by reference in its entirety for all purposes. However, using MS to diagnose cancer has not been demonstrated to date in a clinically relevant manner. What is needed are new biomarkers and new methods of using MS to assess a diagnosis for a disease or a condition, a risk of having a disease or a condition, progression of the disease or condition, and response of the disease or condition to a treatment.

### I. Overview—Immuno-Oncology Response to NSCLC

Described herein are methods for identifying one or more glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) optimizing, by the computer, the predictive model based on performance of the model for a training subset of the data; (d) generating, by the computer, an outcome score for each subject based on the optimized predictive model; and (e) dichotomizing, by the computer, the outcome scores for each subject at a cutoff outcome score as below or above the cutoff outcome score. In some embodiments, the cutoff outcome score was determined to optimize Harrell's C-index. In some embodiments, the cutoff outcome score was determined to optimize hazard ratio.

Provided herein are method for identifying one or more peptide or glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) assessing, by the computer, the predictive model using a cross-validation with n-1 subjects to generate an outcome score for a holdout subject; (d) iterating, by the computer, step (c) for each of n subjects as the holdout subject to generate an outcome score for each subject; (e) dichotomizing, by the computer, the outcome scores for each subject at a cutoff outcome score as below or above the cutoff outcome score; (f) analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide; (g) identifying, by the computer, the glycopeptide having the interaction p-value ≤0.05 as a glycopeptide biomarker for predicting the disease or the condition.

Described herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 1002-1008; (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is non-small-cell lung cancer (NSCLC) and the treatment comprises checkpoint inhibitors. In some embodiments, the outcome comprises overall survival time. In some embodiments, the treatment comprises pembrolizumab therapy. In some embodiments, the treatment comprises pembrolizumab and chemotherapy. In some embodiments, the recommendation comprises continuing the treatment if the output probability indicates the treatment positively influences the outcome.

In some embodiments, provided herein are methods for identifying a classification for a sample, the method comprising: quantifying by mass spectroscopy (MS) one or more glycopeptides in a sample wherein the glycopeptides each, individually in each instance, comprises a glycopeptide consisting essentially of an amino acid sequence selected from the group consisting of SEQ ID NO: 1002-1008; and inputting the quantification into a trained model to generate an output probability; determining if the output probability is above or below a threshold for a classification; and identifying a classification for the sample based on whether the output probability is above or below a threshold for a classification.

In some embodiments, provided herein are methods for training a machine-learning algorithm, comprising: providing a first data set of MRM transition signals indicative of a sample comprising a glycopeptide consisting of, or consisting essentially of, an amino acid sequence selected from the group consisting of SEQ ID NO: 1002-1008; providing a second data set of MRM transition signals indicative of a control sample; and comparing the first data set with the second data set using a machine-learning algorithm.

In some embodiments, provided herein are methods for determining whether a subject would benefit from pembrolizumab therapy; the method comprising: obtaining a biological sample from the patient; performing mass spectrometry of the biological sample using MRM-MS with a QQQ and/or qTOF spectrometer to detect and quantify one or more glycopeptides consisting essentially of an amino acid sequence selected from the group consisting of SEQ ID NO: 1002-1008; or to detect and quantify one or more MRM transitions; inputting the quantification of the detected glycopeptides or the MRM transitions into a trained model to generate an output probability, determining if the output probability is above or below a threshold for a classification; identifying a diagnostic classification for the patient based on whether the output probability is above or below a threshold for a classification; and providing a recommendation for treatment. In some examples, the method includes performing mass spectroscopy of the biological sample using MRM-MS with a QQQ.

### II. Exemplary Descriptions of Terms

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease, including FLD and including NASH, pancreatic cancer, or breast cancer.

The term “a stage of NASH” as used herein refers to a period of progression of NASH from successive phases of severity of fibrosis associated with NASH. As used with respect to the samples encompassed herein, the NASH Clinical Research Network fibrosis staging was for stages F1-F4 corresponding to perisinusoidal fibrosis (F1), periportal fibrosis (F2), bridging fibrosis (F3), and cirrhosis (F4).

The term “F1/F2 stage” as used herein refers to samples or individuals that are either at F1 stage of NASH fibrosis or are at F2 stage of NASH fibrosis.

The term “F3/F4 stage” as used herein refers to samples or individuals that are either at F3 stage of NASH fibrosis or are at F4 stage of NASH fibrosis.

The term “early stage” as used herein in association with NASH refers to samples or individuals that are either at F1 stage of NASH fibrosis or are at F2 stage of NASH fibrosis.

The term “late stage” as used herein in association with NASH refers to samples or individuals that are either at F3 stage of NASH fibrosis or are at F4 stage of NASH fibrosis.

The term “breast cancer state” or “BC state” as used herein refers to the presence in an individual of breast cancer of any type and of any stage. In various embodiments, it refers to ductal or lobular carcinoma; in situ breast cancer, invasive breast cancer, angiosarcoma, Phyllodes tumor, Paget disease of the breast, and so forth.

The term “early stage” as used in association with breast cancer herein refers to stage 1 or stage 2 breast cancer.

The term “pancreatic cancer state” or “PC state” as used herein refers to the presence in an individual of pancreatic cancer of any type and of any stage. In various embodiments, it refers to exocrine pancreatic cancer (including adenocarcinoma (also referred to as ductal carcinoma), squamous cell carcinoma, adenosquamous carcinoma, and colloid carcinoma) and neuroendocrine pancreatic cancer.

The term “early stage” as used in association with pancreatic cancer herein refers to stage 1 or stage 2 pancreatic cancer.

The term “amino acid,” as used herein, generally refers to any organic compound that includes an amino group (e.g., —NH2), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation. N-linked glycosylation can include a glycan attached to an asparagine. O-linked glycosylation can include a glycan attached to either a serine or a threonine.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The terms “biological sex” or “sex” or “sex-related” as used herein refer to a subject being a biological male or a biological female, such as having two X chromosomes for a biological female and an X chromosome and a Y chromosome for a biological male. The terms “biological sex” or “sex” or “sex-related” may be referred to as a gender.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a disease state, a health state, an asymptomatic state, a symptomatic state, etc.). The term “biomarker” may be used interchangeably with the term “marker.”

The term “chronological age” as used herein may refer to the number of years a person has been alive. More particularly, the “chronological age” as used herein may refer to the number of years a person has been alive at the time of a blood draw.

The term “predicted age” as used herein may refer to the age of a subject as based on measurements of one or more peptide structures of Table 23. The predicted age may be of a single year, or a range of years, such as a range of 2, 3, 4, 5, 6, 7, 8, 9, 10, or more years.

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refer to breaking apart a polymer (e.g., cutting a polypeptide at a cut site). Proteins may be digested in preparation for mass spectrometry using trypsin digestion protocols. Proteins may be digested using other proteases in preparation for mass spectrometry if access is limited to cleavage sites.

The terms “immune checkpoint inhibitor therapeutic” and “immune checkpoint inhibitor drug,” as used herein, generally refer to drugs or therapeutics that can target immune checkpoint molecules (e.g., molecules on immune cells that need to be activated (or inactivated) to start an immune response). Non-limiting examples of immune checkpoint inhibitor therapeutics can include pembrolizumab, nivolumab, and cemiplimab.

The term “disease progression,” as used herein, refers to a progression of a disease from no disease or a less advanced (e.g., severe) form of disease to a more advanced (e.g., severe) form of the disease. A disease progression may include any number of stages of the disease. In various embodiments, the term refers to progression of the stages of NASH including F1, F2, F3, and F4. In particular embodiments, the term refers to progression of F1 to F2, F2 to F3, or F3 to F4. In some embodiments, the term refers to progression of early NASH (being in F1 or F2) to late NASH (being in F3 or F4). In various embodiments, the term refers to progression of a healthy, non-NASH state to any stage of NASH, including F1, F2, F3, F4, early NASH, or late NASH.

The term “disease state” as used herein, generally refers to a condition that affects the structure or function of an organism. Disease states can include, for example, stages of a disease progression. For example, for FLD, the progression may be from healthy to a stage of fat accumulation and inflammation (Fatty Liver), to non-alcoholic steatohepatitis (NASH), to fibrosis, and to cirrhosis. Disease states can include any state of a disease whether symptomatic or asymptomatic. Disease states can cause minor, moderate, or severe disruptions in the structure or function of a subject.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refer to a peptide or polypeptide comprising at least one glycan residue. In various embodiments, glycopeptides comprise carbohydrate moieties (e.g., one or more glycans) covalently attached to a side chain (i.e. R group) of an amino acid residue.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins, include but are not limited to Alpha-1-antitrypsin (A1AT), Alpha-2-macroglobulin (A2MG), apolipoprotein C-III (APOC3), alpha-1-antichymotrypsin (AACT), afamin (AFAM), alpha-1-acid glycoprotein 1 & 2 (AGP12), apolipoprotein B-100 (APOB), apolipoprotein D (APOD), complement C1s subcomponent (C1S), calpain-3 (CAN3), clusterin (CLUS), complement component C8AChain (CO8A), alpha-2-HS-glycoprotein (FETUA), haptoglobin (HPT), Histidine-rich Glycoprotein (HRG), Immunoglobulin heavy constant alpha 1 (IGG1), Immunoglobulin heavy constant alpha 2 (IGG2), immunoglobulin heavy constant gamma 1 (IgG1), immunoglobulin J chain (IgJ), plasma kallikrein (KLKB1), serum paraoxonase/arylesterase 1 (PON1), prothrombin (THRB), serotransferrin (TRFE), protein unc-13 homologA (UN13A), and zinc-alpha-2-glycoprotein (ZA2G). A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules by measuring a mass-to-charge (m/z) ratio. In various embodiments described herein, mass spectrometry can be involved in characterization and sequencing of proteins as well as to determine the presence, absence and/or abundance or peptides or proteins.

The term “m/z” or “mass-to-charge ratio” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.” As used herein, the phrase “peptide,” is meant to include glycopeptides unless stated otherwise.

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites. Proteins can include glycoproteins, which are proteins that contain at least one glycan residue bonded thereto.

The term “peptide structure,” as used herein, generally refers to peptides or a portion thereof or glycopeptides or a portion thereof. In various embodiments described herein, a peptide structure can include any molecule comprising at least two amino acids in sequence. A peptide structure of a glycopeptide includes description of the peptide amino acids sequence as well as the location and identity of the associated glycan.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a fluid sample and/or a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ(H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian, or a human. Animals may include, but are not limited to, farm animals, sport animals, and pets. A subject can include a healthy or asymptomatic individual, an individual that has or is suspected of having a disease (e.g., FLD, including NASH) or a pre-disposition to the disease, and/or an individual that needs therapy or suspected of needing therapy. A subject can be a patient. A subject can include a microorganism or microbe (e.g., bacteria, fungi, archaea, viruses). However, in the context of diagnosing ovarian cancer, the subject is female unless explicitly specified otherwise. A subject may be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry. For example, a quadrupole mass analyzer of mass spectrometer can be configured to filter a preselected m/z value that corresponds to a target glycopeptide analyte in an ionized state.

As used herein, a “peptide data set,” may be used interchangeably with “peptide structure data” and can refer to any data of or relating to a peptide from a resulting mass spectrometry run, an ELISA, or western blot. A peptide data set can comprise data obtained from a sample or biological sample using mass spectrometry. A peptide dataset can comprise data relating to a NGEP external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “non-glycosylated endogenous peptide” (“NGEP”), which may also be referred to as an aglycosylated peptide, may refer to a peptide structure that does not comprise a glycan molecule. In various embodiments, an NGEP and a target glycopeptide analyte can originate from the same subject. In various embodiments, an NGEP can be labeled with an isotope in preparation for mass spectrometry analysis.

As used herein, a “transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte may be derived from the same protein sequence. In some embodiments, the NGEP and the target glycopeptide analyte may be derived from or include the same peptide sequence. In various embodiments, a NGEP can be labeled with an isotope in preparation for mass spectrometry analysis.

As used herein, an “abundance value” may refer to “abundance” or a quantitative value associated with abundance.

As used herein, “abundance,” may refer to a quantitative value generated using mass spectrometry. In various embodiments, the quantitative value may relate to an amount of a particular peptide structure (e.g., biomarker) present in a biological sample. In some embodiments, the amount may be in relation to other structures present in the sample (e.g., relative abundance). In some embodiments, the quantitative value may comprise an amount of an ion produced using mass spectrometry. In some embodiments, the quantitative value may be associated with an m/z value (e.g., abundance on x-axis and m/z on y-axis). In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled. In some instances, the term internal standard can be referred to with the abbreviation ISTD.

“Healthy” or “normal” as used herein refers to an individual who does not have NSCLC. The individual may have other diseases, disorders, and/or conditions, which may or may not relate to lung cancer.

“Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

The term “fragment,” as used herein, generally refers to an ion fragmentation process which occurs in a MRM-MS instrument. Fragmenting may produce various fragments having the same mass but varying with respect to their charge, e.g., some biomarkers described herein produce more than one product m/z.

The term “glycopeptide fragment” or “glycosylated peptide fragment” or “glycopeptide” as used herein, generally refers to a glycosylated peptide (or glycopeptide) having an amino acid sequence that is the same as part (but not all) of the amino acid sequence of the glycosylated protein from which the glycosylated peptide is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “therapeutic” may refer generally to any drug that can be administered to a subject physically (e.g., via oral, intravenous injection, topical treatment, exposure, etc.).

The terms “determining”, “measuring”, “evaluating”, “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

As used herein, the terms “cancer” and “cancerous” refer to or describe the physiological condition in a subject that is typically characterized by unregulated cell growth. Examples of cancer include, but are not limited to, melanoma, carcinoma, lymphoma, blastoma, sarcoma, and leukemia and metastases thereof. The term “metastasis” refers to the transference of disease-producing organisms or of malignant or cancerous cells to other parts of the body by way of the blood or lymphatic vessels or membranous surfaces. Non-limiting examples of such cancers include small-cell lung cancer, non-small cell lung cancer, adenocarcinoma of the lung, squamous carcinoma of the lung, melanoma, squamous cell cancer, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, thyroid cancer, hepatic carcinoma and various types of head and neck cancer.

As used herein, the phrase “stage of disease” refers to the stages of cancer progression referred to as Stage I, II, III, or IV. Stage of disease indicates if metastasis has occurred in the subject.

As used herein, the phrase “multiple reaction monitoring mass spectrometry (MRM-MS),” refers to a highly sensitive and selective method for the targeted quantification of glycans and peptides in biological samples. Unlike traditional mass spectrometry, MRM-MS is highly selective (targeted), allowing researchers to fine tune an instrument to specifically look for certain peptides fragments of interest. MRM allows for greater sensitivity, specificity, speed and quantitation of peptides fragments of interest, such as a potential biomarker. MRM-MS involves using one or more of a triple quadrupole (QQQ) mass spectrometer and a quadrupole time-of-flight (qTOF) mass spectrometer.

As used herein, the phrase “digesting a glycopeptide,” refers to a biological process that employs enzymes to break specific amino acid peptide bonds. For example, digesting a glycopeptide includes contacting a glycopeptide with a digesting enzyme, e.g., trypsin, to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing.

As used herein, the phrase “multiple-reaction-monitoring (MRM) transition,” refers to the mass to charge (m/z) peaks or signals observed when a glycopeptide, or a fragment thereof, is detected by MRM-MS. The MRM transition is detected as the transition of the precursor and product ion.

As used herein, the phrase “detecting a multiple-reaction-monitoring (MRM) transition,” refers to the process in which a mass spectrometer analyzes a sample using tandem mass spectrometer ion fragmentation methods and identifies the mass to charge ratio for ion fragments in a sample. The absolute value of these identified mass to charge ratios are referred to as transitions. In the context of the methods set forth herein, the mass to charge ratio transitions are the values indicative of glycan, peptide or glycopeptide ion fragments. For some glycopeptides set forth herein, there is a single transition peak or signal. For some other glycopeptides set forth herein, there is more than one transition peak or signal. Background information on MRM mass spectrometry can be found in Introduction to Mass Spectrometry: Instrumentation, Applications, and Strategies for Data Interpretation, 4th Edition, J. Throck Watson, O. David Sparkman, ISBN: 978-O-470-51634-8, November 2007, the entire contents of which are here incorporated by reference in its entirety for all purposes.

As used herein, the phrase “detecting a multiple-reaction-monitoring (MRM) transition indicative of a glycopeptide,” refers to a MS process in which an MRM-MS transition is detected and then compare to a calculated mass to charge ratio (m/z) of a glycopeptide, or fragment thereof, in order to identify the glycopeptide. In some examples, herein, a single transition may be indicative of two more glycopeptides, if those glycopeptides have identical MRM-MS fragmentation patterns. A transition peak or signal includes, but is not limited to, those transitions set forth herein were are associated with a glycopeptide. A transition peak or signal includes, but is not limited to, those transitions set forth herein are associated with a glycopeptide consisting of an amino acid sequence.

As used herein, the term “reference value” refers to a value obtained from a population of individual(s) whose disease state is known. The reference value may be in n-dimensional feature space and may be defined by a maximum-margin hyperplane. A reference value can be determined for any particular population, subpopulation, or group of individuals according to standard methods well known to those of skill in the art.

As used herein, the term “population of individuals” means one or more individuals. In one embodiment, the population of individuals consists of one individual. In one embodiment, the population of individuals comprises multiple individuals. As used herein, the term “multiple” means at least 2 (such as at least 4, 6, 8, 10, 12, 14, 16, 18, 20, 22, 24, 26, 28, or 30) individuals. In one embodiment, the population of individuals comprises at least 10 individuals.

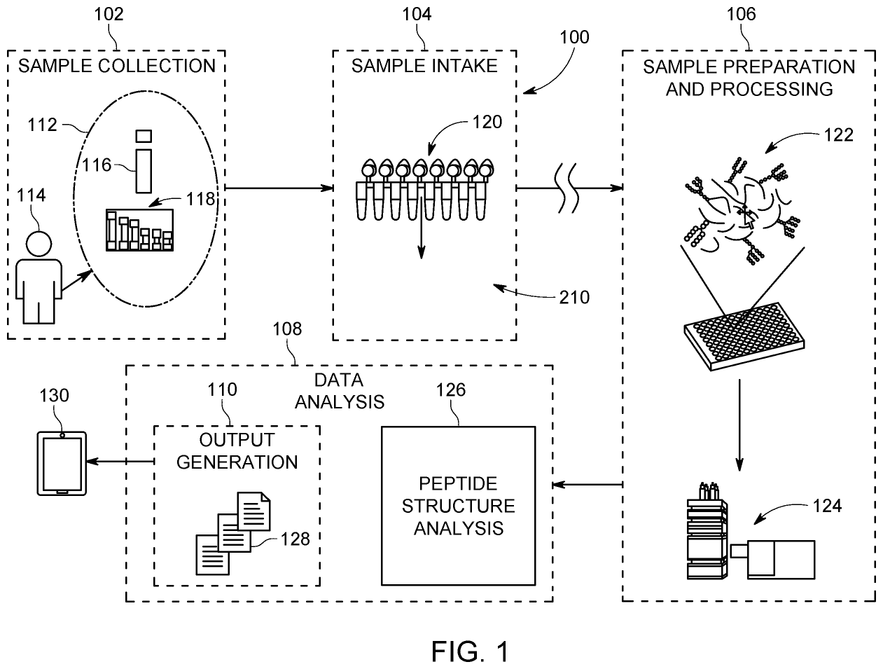

Glycans are referenced herein using the Symbol Nomenclature for Glycans (SNFG) for illustrating glycans. An explanation of this illustration system is available on the internet at www.ncbi.nlm.nih.gov/glycans/snfg.html, the entire contents of which are herein incorporated by reference in its entirety for all purposes. Symbol Nomenclature for Graphical Representation of Glycans as published in Glycobiology 25: 1323-1324, 2015. Additional information showing illustrations of the SNFG system are. Within this system, the term, Hex_i: is interpreted as follows: i indicates the number of green circles (mannose) and the number of yellow circles (galactose). The term, HexNAC_j, uses j to indicate the number of blue squares (GlcNAC's). The term Fuc_d, uses d to indicate the number of red triangles (fucose). The term NeusAC_1, uses 1 to indicate the number of purple diamonds (sialic acid). The glycan reference codes used herein combine these i, j, d, and 1 terms to make a composite 4-5 number glycan reference code, e.g., 5300 or 5320. See, for example, FIGS. 1 through 14 of PCT Patent Application No. PCT/US2020/0162861, filed Jan. 31, 2020, which are herein incorporated by reference in their entirety for all purposes.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an “ex vivo” assay performed on a sample is an “in vitro” assay.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

### III. Overview of Exemplary Workflow

FIG. 1 is a schematic diagram of an exemplary workflow 100 for the detection of peptide structures associated with a disease state for use in diagnosis and/or treatment in accordance with various embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance values (e.g., abundance or raw abundance) for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In various embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. Peptide structure analysis can include determining the composition and the associated quantity for the various peptides and glycopeptides present in the sample by processing the output of a mass spectrometer. In other embodiments, output generation 110 may be considered a separate operation from data analysis 108. Output generation 110 may include, for example, generating final output 128 based on the results of peptide structure analysis 126. In various embodiments, final output 128 may be used for determining the research, diagnosis, and/or treatment of a state associated with fatty liver disease or other cancer.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, the report can comprise a target glycopeptide analyte concentration as a function of the NGEP concentration value and the normalized abundance value. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of, for example, FLD.

### IV. Detection and Quantification of Peptide Structures

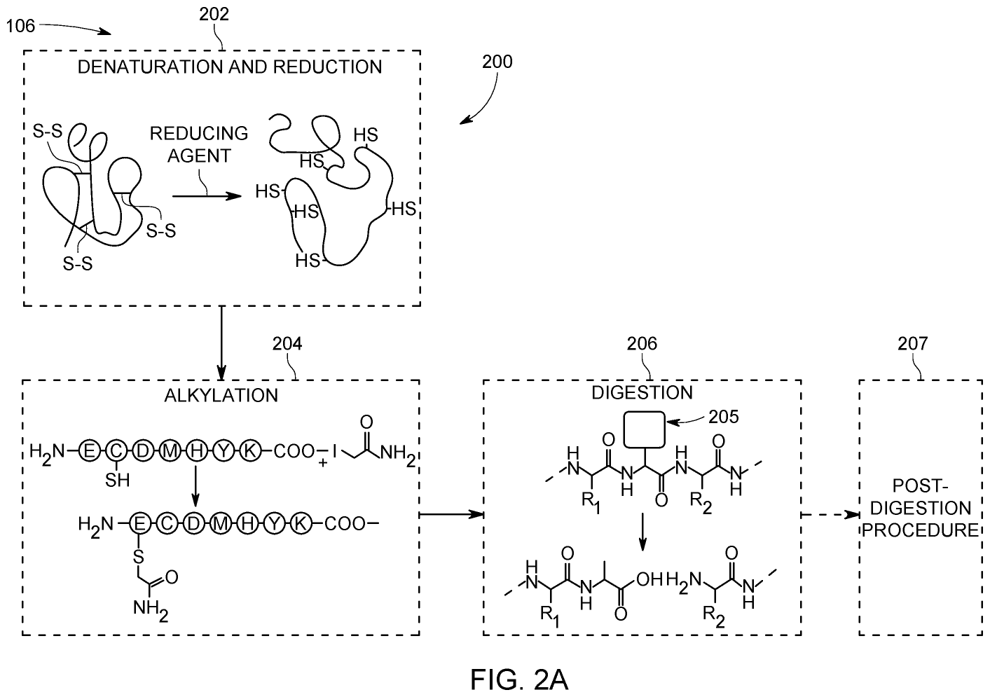

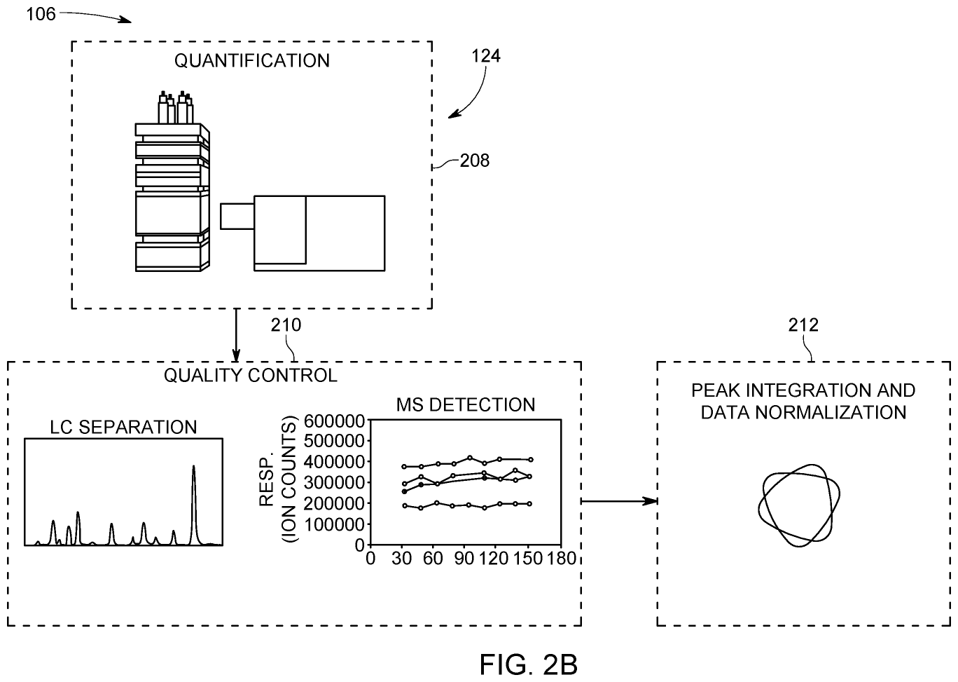

FIGS. 2A and 2B are schematic diagrams of a workflow for sample preparation and processing 106 in accordance with various embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with various embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In various embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents, temperature (e.g., heat), or both. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The acetamide group or alkylation group that attaches to the protein or peptide results in a different form that is not naturally occurring in nature. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In various embodiments, trypsin is used to digest serum samples. In various embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with various embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, LC was performed with gradient elution. The aqueous mobile phase A was 0.1% formic acid in water (vol:vol), and the organic mobile phase B was 0.1% formic acid in acetonitrile (vol:vol). Separation of peptides and glycopeptides was performed using a binary gradient of 0.0-9.0 min, 1-10% B; 9.0-36.0 min, 10-25% B; 36.0-48.0 min, 25-44% B; 48.0-48.1 min, 44-1% B; 48.1-49.0 min, 1% B. The liquid chromatography system can be an Agilent 1290 Infinity II UHPLC system that used a 20 μL loop volume, 4 μL injection volume, Waters ACQUITY UPLC Peptide HSS T3 Column, 100 Å port volume, 1.8 μm particle size, 2.1 mm×150 mm (diameter× length) with HSS T3 guard column, 2.1 mm×5 mm. The output of the chromatography column was either outputted to a waste channel or to the mass spectrometer via an electrospray ionization unit using a microprocessor controlled valve depending on the time of the chromatography run.

In various embodiments, any LC/MS device can be incorporated into the workflow described herein. In various embodiments, an instrument or instrument system suited for identification and targeted quantification 208 may include, for example, a Triple Quadrupole LC/MS. In various embodiments, targeted quantification 208 is performed using multiple reaction monitoring mass spectrometry (MRM-MS). MRM is a mass spectrometry method in which a precursor ion of a particular m/z (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values. The particular m/z value set for the first quadrupole (Q1) and the selected predefined m/z values of the third quadrupole have a mass range that ranges within +/−1, +/−0.5, or +/−0.1 m/z values.

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundance values measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### V. Exemplary System for Peptide Structure Data Analysis

### V.A. Analysis System for Peptide Structure Data Analysis

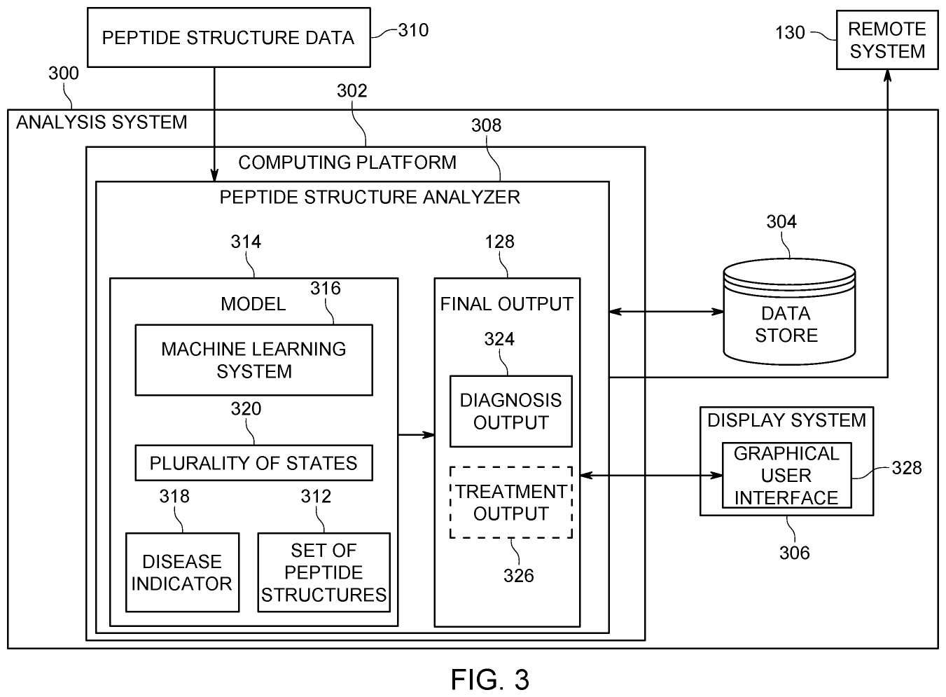

FIG. 3 is a block diagram of an analysis system 300 in accordance with various embodiments. Analysis system 300 can be used to both detect and analyze various peptide structures that have been associated with breast cancer, pancreatic cancer, various sex-associated biomarkers, various age-associated biomarkers, or various states of FLD. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In various embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In various embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure data 310 may include quantification data for the plurality of peptide structures. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In this manner, peptide structure data 310 may provide abundance information about the plurality of peptide structures with respect to biological sample 112.

In various embodiments, peptide structure data 310 may include a set of sex-associated glycosylation biomarkers, and a set of corresponding signals, e.g., the quantification data 316 associated with each of the sex-associated glycosylation biomarkers. The set of corresponding signals, e.g., quantification data 316, is proportional to an amount of each of the sex-associated glycosylation biomarkers in the sample 112. In various embodiments, the set of sex-associated glycosylation biomarkers may include at least one of the sex-associated glycosylation biomarkers listed in Table 28.

In some embodiments, a peptide structure of set of peptide structures 312 comprises a glycosylated peptide structure, or glycopeptide structure, that is defined by a peptide sequence and a glycan structure attached to a linking site of the peptide sequence. For example, the peptide structure may be a glycopeptide or a portion of a glycopeptide. In some embodiments, a peptide structure of set of peptide structures 312 comprises an aglycosylated peptide structure that is defined by a peptide sequence. For example, the peptide structure may be a peptide or a portion of a peptide and may be referred to as a quantification peptide.

Set of peptide structures 312 may be identified as being those most predictive or relevant to the symptomatic disease state based on training of model 314. In various embodiments, set of peptide structures 312 includes at least one, at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, or all 29 of the peptide structures identified in Table 1A. In various embodiments, set of peptide structures 312 includes at least one, at least two, at least three, at least four, at least five, at least six, at least seven, at least eight, at least nine, at least ten, at least 11, at least 12, at least 13, or all of the peptide structures identified in Table 1B. The number of peptide structures selected from Table 1A for inclusion in set of peptide structures 312 may be based on, for example, a desired level of accuracy. In various embodiments, 29 or less peptide structures are selected from Table 1A for inclusion in set of peptide structures 312.

In Table 1A and 113, “PS-ID No.” identifies a label or index for the peptide structure; “Peptide Structure (PS) Name” identifies a name for the peptide structure; “Prot. SEQ ID NO.” identifies the sequence ID of the protein associated with the peptide structure (e.g., from which the peptide structure is derived); “Pep. SEQ ID No. identifies the peptide SEQ ID NO. for the peptide sequence of the peptide structure; “Monoisotopic mass” identifies the monoisotopic mass of the peptide structure in Daltons (Da); “Linking Site Pos. in Prot. Seq.” identifies the site position with respect to the protein sequence at which the corresponding glycan structure is linked; “Linking Site Pos. in Pep. Seq.” identifies the site position with respect to the peptide sequence at which the corresponding glycan structure is linked; and “GL NO.” identifies a label or index for the corresponding glycan structure. For glycopeptide structures, the name for the peptide structure includes an abbreviation of the protein associated with the peptide structure, a first number that corresponds with the linking site position with respect to the protein sequence of the protein, and a second number that identifies the glycan linked to the protein. For aglycosylated peptide structures, the name for the peptide structure includes an abbreviation of the protein associated with the peptide structure and the corresponding peptide sequence of the peptide structure.

TABLE 1A
Various embodiments of Peptide Structures associated with NASH or
Fibrosis Stage Thereof

| | | | | Linking | Linking | |
|---|---|---|---|---|---|---|
| | | | | Site | Site | |
| | | | | Position | Position | Glycan |
| PS- | | | (Peptide) | within | within | Struct. |
| ID | | (Protein) | SEQ ID | Protein | Peptide | GL |
| NO. | PS-NAME | SEQ ID NO. | NO. | Seq | Sequence | NO. |
| PS- | A1AT_271_5402 | 24 | 1 | 271 | 4 | 5402 |
| 01 | | | | | | |
| PS- | A2MG_1424_5402 | 25 | 2 | 1424 | 3 | 5402 |
| 02 | | | | | | |
| PS- | A2MG_1424_NONG | 25 | 2 | — | — | — |
| 03 | LYCOSYLATED | | | | | |
| PS- | AACT_271_7602 | 26 | 3 | 271 | 4 | 7602 |
| 04 | | | | | | |
| PS- | AGP12_72_7604 | 27 and 28 | 4 | 72 | 15 | 7604 |
| 05 | | | | | | |
| PS- | APOB_3895_5401 | 29 | 5 | 3895 | 9 | 5401 |
| 06 | | | | | | |
| PS- | APOC3_74_1101 | 30 | 6 | 94 | 14 | 1101 |
| 07 | | | | | | |
| PS- | HRG_271_2202 | 31 | 7 | 271 | 1 | 2202 |
| 08 | | | | | | |
| PS- | IGA12_144_4401 | 32 and 33 | 8 | 144 | 18 | 4401 |
| 09 | | | | | | |
| PS- | IGG1_297_5410 | 34 | 9 | 180 | 5 | 5410 |
| 10 | | | | | | |
| PS- | IGG2_297_4411 | 35 | 9 | 176 | 5 | 4411 |
| 11 | | | | | | |
| PS- | IGG2_297_5410 | 35 | 9 | 176 | 5 | 5410 |
| 12 | | | | | | |
| PS- | KLKB1_494MC_54 | 36 | 10 | 494 | 6 | 5402 |
| 13 | 02 | | | | | |
| PS- | ANT3_FATTFYQH | 37 | 11 | — | — | — |
| 14 | LADSK | | | | | |
| PS- | FETUA_346_NONG | 38 | 12 | — | — | — |
| 15 | LYCOSYLATED | | | | | |
| PS- | HEMO_187_5401 | 39 | 13 | 187 | 7 | 5401 |
| 16 | | | | | | |
| PS- | APOB_983_5402 | 29 | 14 | 983 | 16 | 5402 |
| 17 | | | | | | |
| PS- | AGP_93_7604 | 27 | 15 | 93 | 7 | 7604 |
| 18 | | | | | | |
| PS- | KLKB1_453_5402 | 36 | 16 | 453 | 7 | 5402 |
| 19 | | | | | | |
| PS- | CO6_324_5402 | 40 | 17 | 324 | 3 | 5402 |
| 20 | | | | | | |
| PS- | THRB_121_5412 | 41 | 18 | 121 | 4 | 5412 |
| 21 | | | | | | |
| PS- | CFAH_529_5402 | 42 | 19 | 529 | 2 | 5402 |
| 22 | | | | | | |
| PS- | APOD_98_5402 | 43 | 20 | 98 | 16 | 5402 |
| 23 | | | | | | |
| PS- | IGG2_297_3500 | 35 | 9 | 176 | 5 | 3500 |
| 24 | | | | | | |
| PS- | IGG2_176_4500 | 35 | 9 | 176 | 5 | 4500 |
| 25 | | | | | | |
| PS- | FHR1_INHGILYDE | 44 | 21 | — | — | — |
| 26 | EK | | | | | |
| PS- | APOC3_74MC_1102 | 30 | 22 | 94 | 16 | 1102 |
| 27 | | | | | | |
| PS- | AGP1_93_6503 | 27 | 15 | 93 | 7 | 6503 |
| 28 | | | | | | |
| PS- | PLASMAFGA_DSH | 45 | 23 | — | — | — |
| 29 | SLTTNIMEILR | | | | | |

TABLE 1B
Various embodiments of Peptide Structures associated with NASH or Fibrosis Stage Thereof

| | | | | Linking | Linking | |
|---|---|---|---|---|---|---|
| | | | | Site | Site | |
| | | | | Position | Position | |
| | | (Prot) | (Pept) | within | within | Glycan |
| PS- | PS- | SEQ ID | SEQ ID | Protein | Peptide | Struct. |
| ID NO. | NAME | NO. | NO. | Sequence | Sequence | GL NO. |
| PS-01 | A1AT_271_5402 | 24 | 1 | 271 | 4 | 5402 |
| PS-02 | A2MG_1424_5402 | 25 | 2 | 1424 | 3 | 5402 |
| PS-03 | A2MG_1424_NONGLYCOSYLATED | 25 | 2 | — | — | — |
| PS-04 | AACT_271_7602 | 26 | 3 | 271 | 4 | 7602 |
| PS-05 | AGP12_72_7604 | 27 and 28 | 4 | 72 | 15 | 7604 |
| PS-06 | APOB_3895_5401 | 29 | 5 | 3895 | 9 | 5401 |
| PS-07 | APOC3_74_1101 | 30 | 6 | 94 | 14 | 1101 |
| PS-08 | HRG_271_2202 | 31 | 7 | 271 | 1 | 2202 |
| PS-09 | IGA12_144_4401 | 32 and 33 | 8 | 144 | 18 | 4401 |
| PS-10 | IGG1_297_5410 | 34 | 9 | 180 | 5 | 5410 |
| PS-11 | IGG2_297_4411 | 35 | 9 | 176 | 5 | 4411 |
| PS-12 | IGG2_297_5410 | 35 | 9 | 176 | 5 | 5410 |
| PS-13 | KLKB1_494MC_5402 | 36 | 10 | 494 | 6 | 5402 |
| PS-14 | ANT3_FATTFYQHLADSK | 37 | 11 | — | — | — |

Key for Table 1A and 1B and elsewhere herein:
nonglycosylated: a variant of a glycopeptide that is without glycosylation MC: miscleavage (a peptide of a different length from its counterpart that lacks the MC notation)
plasma: a fibrinogen peptide that assists in identification whether a sample is plasma or serum (appears in both plasma and serum samples but is upregulated in plasma samples)

In various embodiments, set of peptide structures 312 includes only peptide structures fragmented from Alpha-1-antitrypsin (A1AT) and thus only A1AT glycoforms. In various embodiments, set of peptide structures 312 includes only peptide structures fragmented from alpha-2-macroglobulin (A2MG) and thus only A2MG glycoforms. In various embodiments, set of peptide structures 312 includes only peptide structures fragmented from the Alpha-1-antichymotrypsin (AACT) and thus only AACT glycoforms. In various embodiments, set of peptide structures 312 includes only peptide structures fragmented from Alpha-1-acid glycoprotein 1 and/or Alpha-1-acid glycoprotein 2 (AGP12) and thus only AGP12 glycoforms. In various embodiments, set of peptide structures 312 includes only peptide structures fragmented from Apolipoprotein B-100 (APOB) and thus only APOB glycoforms. In various embodiments, set of peptide structures 312 includes only peptide structures fragmented from Apolipoprotein C-III (APOC3) and thus only APOC3 glycoforms. In various embodiments, set of peptide structures 312 includes only peptide structures fragmented from Histidine-rich Glycoprotein (HRG) and thus only HRG glycoforms. In various embodiments, set of peptide structures 312 includes only peptide structures fragmented from Immunoglobulin heavy constant alpha 1 and/or 2 (IGA12) and thus only IGA12 glycoforms. In various embodiments, set of peptide structures 312 includes only peptide structures fragmented from Immunoglobulin heavy constant gamma 1 (IGG1) and thus only IGG1 glycoforms. In various embodiments, set of peptide structures 312 includes only peptide structures fragmented from Immunoglobulin heavy constant gamma 2 (IGG2) and thus only IGG2 glycoforms. In various embodiments, set of peptide structures 312 includes only peptide structures fragmented from Plasma Kallikrein (KLKB1) and thus only KLKB1 glycoforms. In various embodiments, set of peptide structures 312 includes only peptide structures fragmented from Antithrombin-III. In some embodiments, set of peptide structures 312 includes only peptide structures fragmented from at least one of A1AT, A2MG, AACT, AGP12, APOB, APOC3, HRG, IGA12 IGG1, IGG2, KLKB1, or Antithrombin-III.

Peptide structure analyzer 308 includes model 314 that is configured to receive peptide structure data 310 for processing. Model 314 may be implemented in any of a number of different ways. Model 314 may be implemented using any number of models, functions, equations, algorithms, and/or other mathematical techniques.

In various embodiments, model 314 includes machine learning model 316, which may itself be comprised of any number of machine learning models and/or algorithms. For example, machine learning model 316 may include, without limitation, at least one of a parametric model, a non-parametric model, deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a logistic regression model, a multivariable regression model, a penalized multivariable regression model, or another type of model. In various embodiments, model 314 includes a machine learning model 316 that comprises any number of or combination of the models or algorithms described above.

In various embodiments, model 312 analyzes peptide structure data 310 for each sample of a cohort to generate a predicted age 324 associated for each sample, where each sample corresponds to a subject and has an associated chronological age or range of ages 315. In various embodiments, peptide structure data 310 may include quantification data 316 for the plurality of peptide structures 314 (also referred to herein as structure markers 314). Quantification data 316 for a peptide structure can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to sample 112. Each of the age-associated glycosylation biomarkers of the set can include a precursor m/z value and/or a product ion m/z value.

In various embodiments, peptide structure data 310 may include a set of age-associated glycosylation biomarkers, and a set of corresponding signals, e.g., the quantification data 316 associated with each of the age-associated glycosylation biomarkers. The set of corresponding signals, e.g., quantification data 316, is proportional to an amount of each of the age-associated glycosylation biomarkers in the sample 112. In various embodiments, the set of age-associated glycosylation biomarkers may include at least one of the age-associated glycosylation biomarkers listed in Table 23.

In various embodiments, model 314 analyzes the portion (e.g., some or all of) peptide structure data 310 corresponding set of peptide structures 312 to generate disease indicator 318 that classifies biological sample 112 as evidencing a corresponding state of a plurality of states 320 associated with FLD progression. Disease indicator 318 may take various forms. In various embodiments, disease indicator 318 is a score that indicates a classification of the corresponding state for biological sample 112. For example, each of the states 320 may be associated with a different range of values for the score. If the score falls within a selected range associated with a particular state of the states 320, then the score indicates that biological sample 112 evidences that particular state. Thus, the score provides a classification of biological sample 112 as corresponding to that particular state.

In other embodiments, disease indicator 318 includes a score that indicates a probability that a subject (e.g., subject 114 in FIG. 1) falls within one of the states 320 associated with FLD progression. For example, disease indicator 318 may include one or more scores, each of which may indicate whether biological sample 112 evidences a corresponding state of the states 320 associated with FLD progression. In some examples, disease indicator 318 includes a score for each of the states 320 associated with FLD progression. A higher score indicates a higher probability that biological sample 112 evidences the corresponding state. In various embodiments, states 320 include a NASH state, a non-NASH state, an early stage NASH state, or a late stage NASH state.

In various embodiments, machine learning model 316 takes the form of regression model 320. Regression model 320 may include, for example, at least one LASSO regression model (or LASSO regularization model) that is trained to compute disease indicator 318. Regression model 320 may be trained to identify weight coefficients for peptide structures of set of peptide structures 312.

Peptide structure analyzer 308 may generate final output 128 based on disease indicator 318 that is output by model 314. In other embodiments, final output 128 may be an output generated by model 314.

In some embodiments, final output 128 includes disease indicator 318. In other embodiments, final output 128 includes diagnosis output 324 and/or treatment output 326. Diagnosis output 324 may include, for example, an identification of a classification of which of the states 320 evidenced by biological sample 112 based on disease indicator 318. Treatment output 326 may include, for example, at least one of an identification of a therapeutic to treat the subject, a design for the therapeutic, or a treatment plan for administering the therapeutic. In some embodiments, the therapeutic is an immune checkpoint inhibitor.

Final output 128 may be sent to remote system 130 for processing in some examples. In other embodiments, final output 128 may be displayed on graphical user interface 328 in display system 306 for viewing by a human operator. The human operator may use final output 128 to diagnose and/or treat subject when final output 128 indicates the subject is positive a state (e.g., NASH or a certain stage thereof) along a disease progression of a disease (e.g., FLD).

In various embodiments, model 314 analyzes peptide structure data 310 to generate disease indicator 318 that indicates whether the biological sample is positive for a breast cancer (BC) disease state based on set of peptide structures 312 identified as being associated with the BC disease state. Peptide structure data 310 may include quantification data for the plurality of peptide structures. Quantification data for peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to biological sample 112.

Disease indicator 318 may take various forms. In some examples, disease indicator 316 includes a classification that indicates whether or not the subject is positive for the BC disease state. In various embodiments, disease indicator 318 can include a score. Score indicates whether the BC disease state is present or not. For example, score may be a probability score that indicates how likely it is that the biological sample 112 evidences the presence of the BC disease state.

In some embodiments, a peptide structure of set of peptide structures 312 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 312 comprises an aglycosylated peptide structure that is defined by a peptide sequence. For example, the peptide structure may be a peptide or a portion of a peptide and may be referred to as a quantification peptide.

Set of peptide structures 312 may be identified as being those most predictive or relevant to the BC disease state based on training of model 314. In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, or all 18 of the peptide structures identified in Table 9 below. In some cases, the number of peptide structures selected from Table 9 for inclusion in set of peptide structures 318 may be based on, for example, a desired level of accuracy.

In various embodiments, set of peptide structures 312 may be identified as being those most predictive or relevant to the BC disease state based on training of model 314. In one or more embodiments, set of peptide structures 312 includes at least 1, at least 2, at least 3, at least 4, at least 5, at least 6, or all 7 of the peptide structures identified in Table 10A below. In some cases, the number of peptide structures selected from Table 10A for inclusion in set of peptide structures 312 may be based on, for example, a desired level of accuracy.

In various embodiments, set of peptide structures 312 may be identified as being those most predictive or relevant to the BC disease state based on training of model 314. In one or more embodiments, set of peptide structures 312 includes at least 1, at least 2, at least 3, at least 4, at least 5, at least 6, at least 7, or all 8 of the peptide structures identified in Table 10B below. In some cases, the number of peptide structures selected from Table 10B for inclusion in set of peptide structures 312 may be based on, for example, a desired level of accuracy.

In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, or all 18 of the peptide structures PS-30 through PS-47 in Table 9.

In one or more embodiments, set of peptide structures 312 includes at least 1, at least 2, at least 3, at least 4, at least 5, at least 6, or all 7 of the peptide structures PS-33, PS-42, PS-44, PS-30, PS-47, PS-43, or PS-37 in Table 10A.

In one or more embodiments, set of peptide structures 312 includes at least 1, at least 2, at least 3, at least 4, at least 5, at least 6, at least 7, or all 8 of the peptide structures PS-42, PS-33, PS-41, PS-43, PS-47, PS-37, PS-30, or PS-45 in Table 10B.

Set of peptide structures 312 may be identified as being those most predictive or relevant to the PC disease state based on training of model 314. In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, or all 55 of the peptide structures identified in Table 16. In some cases, the number of peptide structures selected from Table 16 for inclusion in set of peptide structures 312 may be based on, for example, a desired level of accuracy.

In various embodiments, set of peptide structures 312 may be identified as being those most predictive or relevant to the PC disease state based on training of model 314. In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures identified in Table 17A below. In some cases, the number of peptide structures selected from Table 17A for inclusion in set of peptide structures 312 may be based on, for example, a desired level of accuracy.

In various embodiments, set of peptide structures 312 may be identified as being those most predictive or relevant to the PC disease state based on training of model 314. In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or all 19 of the peptide structures identified in Table 17B below. In some cases, the number of peptide structures selected from Table 17B for inclusion in set of peptide structures 312 may be based on, for example, a desired level of accuracy.

In various embodiments, set of peptide structures 312 may be identified as being those most predictive or relevant to the PC disease state based on training of model 314. In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, or all 17 of the peptide structures identified in Table 17C below in Section VIA. In some cases, the number of peptide structures selected from Table 17C for inclusion in set of peptide structures 312 may be based on, for example, a desired level of accuracy.

In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, or all 55 of the peptide structures PS-48 through PS-102 in Table 16.

In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures PS-49, PS-50, PS-54, PS-61, PS-63, PS-64, PS-71, PS-79, PS-81, PS-84, PS-86, PS-87, PS-90, PS-91, PS-92, PS-94, PS-95, PS-96, PS-97, PS-98, PS-99, or PS-101 in Table 17A.

In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or all 19 of the peptide structures PS-48, PS-52, PS-57, PS-61, PS-62, PS-63, PS-64, PS-69, PS-71, PS-72, PS-73, PS-84, PS-86, PS-88, PS-91, PS-94, PS-96, PS-100, or PS-101 in Table 17B.

In one or more embodiments, set of peptide structures 312 includes at least 1, at least 2, at least 3, at least 4, at least 5, at least 6, at least 7, at least 8, at least 9, at least 10, at least 11, at least 12, at least 13, at least 14, at least 15, at least 16, or all 17 of the peptide structures PS-48, PS-52, PS-61, PS-64, PS-66, PS-68, PS-69, PS-71, PS-72, PS-73, PS-86, PS-89, PS-91, PS-94, PS-96, PS-99, or PS-101 in Table 17C.

In various embodiments, machine learning system 316 takes the form of binary classification model. Binary classification model may include, for example, but is not limited to, a regression model. Binary classification model may include, for example, a penalized multivariable regression model that is trained to identify set of peptide structures 312 from a plurality of (or panel of) peptide structures identified in various subjects. Binary classification model may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 312.

Peptide structure analyzer 308 may generate final output 128 based on disease indicator 318 output by model 314. In other embodiments, final output 128 may be an output generated by model 314.

In some embodiments, final output 128 includes disease indicator 318. In other embodiments, final output 128 includes diagnosis output 324, treatment output 326, or both. Diagnosis output 324 may include, for example, a diagnosis for the PC disease state. The diagnosis can include a positive diagnosis or a negative diagnosis for the PC disease state. In one or more embodiments, generating diagnosis output 324 may include comparing score to selected threshold to determine the diagnosis. Selected threshold may be, for example, without limitation, (e.g., 0.4, 0.5, 0.6, etc.). For example, when selected threshold is set to 0.5, a score above 0.5 may indicate the presence of the PC disease state and be output in diagnosis output 324 as a positive diagnosis. Treatment output 326 may include, for example, at least one of an identification of a treatment for the subject, a treatment plan for administering the treatment, or both. Treatment for pancreatic cancer may include, for example, but is not limited to, at least one of radiation therapy, chemoradiotherapy, surgery, a targeted drug therapy, or some other form of treatment. The treatment plan may include, for example, but is not limited to, a timeline or schedule for administering the treatment, dosing information, other treatment-related information, or a combination thereof.

Final output 128 may be sent to remote system 130 for processing in some examples. In other embodiments, final output 128 may be displayed on graphical user interface 328 in display system 306 for viewing by a human operator

In various embodiments, machine learning system 316 takes the form of binary classification model. Binary classification model may include, for example, but is not limited to, a regression model. Binary classification model may include, for example, a penalized multivariable regression model that is trained to identify set of peptide structures 312 from a plurality of (or panel of) peptide structures identified in various subjects. Binary classification model may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 312.

Peptide structure analyzer 308 may generate final output 128 based on disease indicator 318 output by model 314. In other embodiments, final output 128 may be an output generated by model 314.

In some embodiments, final output 128 includes disease indicator 318. In other embodiments, final output 128 includes diagnosis output 324, treatment output 326, or both. Diagnosis output 324 may include, for example, a diagnosis for the BC disease state. The diagnosis can include a positive diagnosis or a negative diagnosis for the BC disease state. In one or more embodiments, generating diagnosis output 324 may include comparing score to selected threshold to determine the diagnosis. Selected threshold may be, for example, without limitation, (e.g., 0.4, 0.5, 0.6, etc.). For example, when selected threshold 328 is set to 0.5, a score 320 above 0.5 may indicate the presence of the BC disease state and be output in diagnosis output 324 as a positive diagnosis. Treatment output 326 may include, for example, at least one of an identification of a treatment for the subject, a treatment plan for administering the treatment, or both. Treatment for breast cancer may include, for example, but is not limited to, at least one of radiation therapy, chemoradiotherapy, surgery, a targeted drug therapy, or some other form of treatment. The treatment plan may include, for example, but is not limited to, a timeline or schedule for administering the treatment, dosing information, other treatment-related information, or a combination thereof. In various embodiments, the set of peptide structures 314 may include a set of biomarkers identified as being those most relevant to predicting a biological age or range of age of one or more subjects from whom the sample(s) was taken. In one or more embodiments, set of peptide structures 314 includes at least one, at least two, or at least three peptide structures from a first group of peptide structures (peptide structures PS-103 through PS-122) identified in Table 23. For example, in one or more embodiments, set of peptide structures 314 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or all 20 of the peptide structures identified in Table 23. In some cases, the number of peptide structures selected from Table 23 for inclusion in the set of peptide structures 314 may be based on, for example, a desired level of accuracy.

In various embodiments, principal component analysis (PCA) 318 can be performed to determine most relevant biomarkers from the set of peptide structures 314. Principal Component Analysis (PCA) is a mathematical technique used to describe the variance in the dataset with a relatively small number of Principal Components vectors (PCs). In various embodiments, principal component analysis 318 may be performed to determine one or more PCA features for each glycopeptide group of the set of peptide structures 314. A glycopeptide group can have a plurality of glycoforms where each glycoform has the same peptide sequence with a different glycan structure attached to the same amino acid location within the peptide sequence. A set of glycopeptide groups can represent various peptide sequences and their associated glycoforms. The PCA features are vectors explaining the variance across the various glycoforms for a glycopeptide amino acid site. PCA can be performed for a plurality of proteins typically found in a biological sample like serum. After performing PCA on glycopeptide site level features (i.e., glycopeptide group_, a set of three Principle components vector features for each glycopeptide site that are used to perform further downstream analysis. The PCA feature vectors are extracted taking into consideration the concentration values of all unique glycopeptide sites and the group of glycoforms associated with that specific site. For example, a “specific site” is in reference to the amino acid residue at which the glycan is attached. The “specific site” can have a different type of attached glycan structure, where in many instances there can be 3 or more glycoforms. PCA can be used to collapse that information across all N dimensions into 3 PC's per site, that represent the top three axes of independent information. For example, a group of glycoforms can be represented by the protein CFAH where the glycans attach at a specific amino acid site 1029 (with respect to the UniProt protein sequence) and there are three possible glycans that can attach to the specific site 1029 (e.g., glycan 5401, 5402, and 5412). For example, the first three PC vectors which explain the largest variance for a group of glycoforms for a specific glycopeptide site can be extracted. Each PC vector is a statistical measure of variation explained at a specific glycosylation site starting from PC1 being the highest explained variation to PC3 being the lowest. The table below lists PC vectors that are generated after performing PCA on each glycopeptide site and the glycoforms associated with that site. In one embodiment, PCA analysis was performed on a plurality of glycopeptide groups that resulted in one to three PC vectors for each glycopeptide group. For example, three vectors for one glycopeptide group (where the peptide is part of the protein CFAH and that various glycoforms are attached at amino acid position 1029) could be represented by PC1_1029_CFAH, PC2_1029_CFAH, PC3_1029_CFAH where the terms:
PC1 represents—principal component 1 vector
PC2 represents—principal component 2 vector
PC3 represents—principal component 3 vector. The term PC vector may also be referred to as a PCA feature.

As noted above, PCA features were determined with a PCA analysis on a plurality of glycopeptide groups. In various embodiments, linear regression was performed with the PCA features and the chronological age for each of the samples. In various embodiments, the set of the PCA features that have significant values below a certain threshold value. A list of all the PC vector features that are significant with a p-value of less than 0.05 are as follows: PC3_297_IGG1; PC3_297_IGG2; PC1_352_IC1; PC1_128_ZA2G; PC3_205_IGA2; PC3_72_AGP12; PC3_144_IGA12; PC2_253_IC1; PC2_144MC_IGA12; PC2_630_TRFE; PC3_138_CERU; and PC3_209_IGM. In various embodiments, statistically significant values for each of the PCA features may be an output of the linear regression.

A list of 12 glycopeptide groups were found to be significant (p<0.05). By accounting for the various glycoforms for each of the 12 glycopeptide groups (297_IGG1; 297_IGG2; 352_IC1; 128_ZA2G; 205_IGA2; 72_AGP12; 144_IGA12; 253_IC1; 144MC_IGA12; 630_TRFE; 138_CERU; and 209_IGM), a total of 20 glycopeptide biomarkers were identified that can be used for logistic regression modeling via model learning system 320.

In various embodiments, machine learning system 320 can be trained using one or more glycopeptides for each glycopeptide group of the selected set of the one or more PCA features and the chronological age for each sample. In various embodiments, machine learning system 320 can be tested using another cohort of samples, which can generate predicted age values and compare against the chronological age values of that cohort of samples for validation.

In various embodiments, machine learning system 320 takes the form of binary classification model 322. Binary classification model 322 may include, for example, but is not limited to, a regression model. Binary classification model 322 may include, for example, a penalized multivariable regression model that is trained to identify a set of peptide structures 314 from a plurality of (or panel of) peptide structures identified in various subjects. Binary classification model 322 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 314.

In various embodiments, machine learning system 320 may include multiplying the corresponding signal, e.g., quantification data 316, associated with age-associated glycosylation biomarkers and a respective coefficient for each sample of the cohort to form a plurality of products. In various embodiments, machine learning system 320 may include summing together the plurality of products to form a summation, and then adding the summation and the intercept to form an output value, where the output value is proportional to the predicted age for the sample.

In various embodiments, machine learning system 320 may include multiplying the corresponding signal, e.g., quantification data 316, associated with sex-associated glycosylation biomarkers and a respective coefficient for each sample of the cohort to form a plurality of products. In various embodiments, machine learning system 320 may include summing together the plurality of products to form a summation, and then adding the summation and the intercept to form an output value, where the output value is greater than a first threshold to determine a sex associated with the sample or subject such as male or female.

In various embodiments, machine learning system 320 may include an equation to determine an output value as follows:

$$O V = {\sum }_{i = 1}^{i = 20} [ ( {Signal}_{SEQ ID No : i} ) \times ( {Coefficient}_{SEQ ID No : i} ) ] + Intercept$$

$$where O V = an output value ,$$

$$i = an index number for each of the age - associated glycosylation biomarkers ,$$

$${Signal}_{SEQ ID No : i} = a corresponding signal or quantification data associated with the age - associated glycosylation biomarker i , and$$

$${Coefficient}_{SEQ ID No : i} = a coefficient associated with the age - associated glycosylation biomarker i .$$

In various embodiments, peptide structure analyzer 308 may generate final output 128 based on the PCA features 319 output by model 312. In other embodiments, final output 128 may be an output generated by model 312. In some embodiments, final output 128 includes PCA features 319. In various embodiments, final output 128 includes a predicted age 324. In various embodiments, final output 128 includes a correlation coefficient 328. In various embodiments, correlation coefficient 328 is a Pearson correlation coefficient where the predicted age 324 is a continuous variable and the chronological age 315 is another continuous variable. The Pearson correlation coefficient (r_{xy}) can be determined using the following equation:

$${r}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$

$$where n = a number of the samples in the cohort ,$$

$$i = an index number for each of the samples ,$$

$${x}_{i} = a chronological age for sample i ,$$

$$\overline{x} = a mean chronological age ,$$

$${y}_{i} = a predicted age for sample i , and$$

$$\overline{y} = a mean predicted age .$$

In various embodiments, correlation coefficient 328 determined based on the predicted age 324 and the chronological age 315 for each sample can help identify a quality control issue associated with the chronological age provided with the sample. For example, if correlation coefficient 328 does not fall within a predetermined range of values 326, final output 128 can include a quality control issue associated with the chronological age for the cohort or that particular batch of samples. In various embodiments, the predetermined range of values 326 can range from about 0 to about 0.2. In various embodiments, the quality control issue identified as final output 128 can include, for example, an error of mislabeled samples or an error from sample preparation, or a systemic measurement or an instrument error, or a combination thereof.

In various embodiments, machine learning system 320 may include multiplying the corresponding signal, e.g., quantification data 316, associated with sex-associated glycosylation biomarkers and a respective coefficient for each sample of the cohort to form a plurality of products. In various embodiments, machine learning system 320 may include summing together the plurality of products to form a summation, and then adding the summation and the intercept to form an output value, where the output value is greater than a first threshold to determine a sex associated with the sample or subject such as male or female.

In various embodiments, machine learning system 320 may include an equation to determine an output value as follows:

$$O V = {\sum }^{i = 42} \qquad {SEQ ID No : i}_{} \qquad {SEQ ID No : i}_{}$$

$$where O V = an output value ,$$

$$i = an index number for each of the sex - associated glycosylation biomarkers ,$$

$${SEQ ID No : i}_{} = a corresponding signal or quantification data associated with the sex - associated glycosylation biomarker i , and$$

$${SEQ ID No : i}_{} = a coefficient associated with the sex - associated glycosylation biomarker i .$$

In various embodiments, the above OV equation can be used as model 312 that uses quantification data 316 for calculating the output value OV, In one embodiment, the subject or sample can be estimated to have the sex of male when the output value OV is greater than a first threshold and the subject or sample can be estimated to have the sex of female when the output value OV is less than the first threshold. In various embodiments, the OV can be a probability value and where the probability is less than or equal to 0.5 (e.g., first threshold), a binary classification method can then output a 0 that corresponds to a male. Similarly, where the probability is greater than 0.5, the binary classification method can then output a 1 that corresponds to a female.

In various embodiments, peptide structure analyzer 308 may generate final output 128 based on the PCA features 319 output by model 312. In other embodiments, final output 128 may be an output generated by model 312. In some embodiments, final output 128 includes PCA features 319. In various embodiments, final output 128 includes a predicted sex 324. In various embodiments, final output 128 includes an accuracy score 327.

In various embodiments, accuracy score 327 can be determined based on the predicted sex 324 and the annotated sex 315 for each sample. For example, the accuracy score can be based on a ratio of the number of samples of a cohort that had the same gender for predicted sex and annotated sex and the total number of samples of the cohort. More particularly, the accuracy score can be the number of samples of a cohort that had the same gender for the predicted sex and annotated sex divided by the total number of samples of the cohort. In various embodiments, the determined accuracy score 327 can help identify a quality control issue associated with the annotated sex provided with the sample. For instance, when the determined accuracy score 327 is less than a threshold value (e.g., 0.5), then a quality control issue can be provided for the analysis of the cohort that may be related to a mistake in inputting the gender for one or more subjects. For example, depending on the accuracy score 327, final output 128 can include a quality control issue associated with the annotated sex for the cohort or that particular batch of samples. In various embodiments, the quality control issue identified as final output 128 can include, for example, an error of mislabeled samples or an error from sample preparation, or a systemic measurement or an instrument error.

In various embodiments, final output 128 includes the sensitivity score 328 and/or the specificity score 329. In various embodiments, the sensitivity score 328 and/or the specificity score 329 can be determined based on the predicted sex 324 and the annotated sex 315 for each sample in the cohort. In various embodiments, the determined sensitivity score 328 and specificity score 329 illustrate the overall performance of the model in regard to the true positive rate and the true negative rate, respectively.

### V.B. Analysis System for Peptide Structure Data Analysis for Diagnosis of NSCLC

FIG. 3 is a block diagram of an analysis system 300, in accordance with the presently disclosed embodiments. For example, in accordance with the presently disclosed embodiments, the analysis system 300 may include any computing platform that may be utilized for performing one or more methods of classifying a biological sample obtained from a subject with respect to a plurality of states associated with NSCLC, detecting the presence of one of a plurality of states associated with NSCLC, treating NSCLC in a subject, determining techniques for treating NSCLC in a subject, diagnosing an individual with NSCLC, and training a model to diagnose a subject with one of a plurality of states associated with NSCLC, in accordance with the presently disclosed embodiments. Analysis system 300 can be used to detect and analyze various peptide structures that have been associated with various states of NSCLC. Analysis system 300 is one example of an implementation for a system that may be used to perform data analysis 108. Analysis system 300 may include computing platform 302 and data store 304.

In certain embodiments, analysis system 300 may also include display system 306. Computing platform 302 may take various forms. In certain embodiments, computing platform 302 may include a single computer (or computer system) or multiple computers in communication with each other. In other examples, computing platform 302 takes the form of a cloud computing platform. Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

In certain embodiments, analysis system 300 may include, for example, peptide structure analyzer 308, which may be implemented using hardware, software, firmware, or a combination thereof. In certain embodiments, peptide structure analyzer 308 is implemented using computing platform 302. Peptide structure analyzer 308 receives peptide structure data 310 for processing. Peptide structure data 310 may be, for example, the peptide structure data that is output from sample preparation and processing 106 in FIG. 1, FIG. 2A, and FIG. 2B. Accordingly, peptide structure data 310 may correspond to set of peptide structures 122 identified for biological sample 112 and may thereby correspond to biological sample 112. Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device. Peptide structure data 310 may include quantification data for the plurality of peptide structures. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In this manner, peptide structure data 310 may provide abundance information about the plurality of peptide structures with respect to biological sample 112.

In certain embodiments, a peptide structure of set of peptide structures 312 may include a glycosylated peptide structure, or glycopeptide structure, that is defined by a peptide sequence and a glycan structure attached to a linking site of the peptide sequence. For example, the peptide structure may be a glycopeptide or a portion of a glycopeptide. In certain embodiments, a peptide structure of set of peptide structures 312 may include an aglycosylated peptide structure that is defined by a peptide sequence. For example, the peptide structure may be a tag glycopeptide or a portion of a tag glycopeptide and may be referred to as a quantification peptide. A tag peptide can be a peptide with at least one isotopically labeled amino acid. Set of peptide structures 312 may be identified as being those most predictive or relevant to the symptomatic disease state based on training of model 314. In certain embodiments, set of peptide structures 312 may include at least one, at least two, at least three, at least four, at least five, at least 10, at least 15, at least 20, at least 25, or all of the peptide structures identified in Table 35 below. The number of peptide structures selected from Table 35 for inclusion in set of peptide structures 312 may be based on, for example, a desired level of accuracy. In certain embodiments, an N number of peptide structures may be selected from Table 35 for inclusion in set of peptide structures 312, in which Nis an integer from 1-73. In certain embodiments, set of peptide structures 312 may include at least one, at least two, at least three, at least four, at least five, at least 10, at least 15, or all of the peptide structures identified in Table 40 below. The number of peptide structures selected from Table 40 for inclusion in set of peptide structures 312 may be based on, for example, a desired level of accuracy. In certain embodiments, an N number of peptide structures may be selected from Table 35 for inclusion in set of peptide structures 312, in which Nis an integer from 1-19.

Peptide structure analyzer 308 may include model 314 that may be able to receive peptide structure data 310 for processing. Model 314 may be implemented in any of a number of different ways. Model 314 may be implemented using any number of models, functions, equations, algorithms, and/or other mathematical techniques. In certain embodiments, model 314 may include one or more machine-learning systems 316, which may include any number of machine-learning models and/or algorithms. For example, machine-learning systems 316 may include, without limitation, at least one of a parametric model, a non-parametric model, deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, a supervised machine-learning model, an unsupervised machine-learning model, a logistic regression model, a univariate regularized regression model (e.g., a univariate least absolute shrinkage and selection operator (LASSO) regression model), a multivariate regularized regression model (e.g., a multivariate LASSO regression model), a penalized univariate regularized regression model (e.g., a penalized univariate LASSO regression model), a penalized multivariate regularized regression model (e.g., a penalized multivariate LASSO regression model), or any of various other models that may be utilized to predict the presence and/or absence of NSCLC in a subject, in accordance with the presently-disclosed embodiments. In certain embodiments, model 314 may include machine-learning systems 316, which may include any number of or combination of the models or algorithms described above.

In certain embodiments, model 314 analyzes the portion (e.g., some or all of) peptide structure data 310 corresponding set of peptide structures 312 to generate disease indicator 318 that classifies biological sample 112 as evidencing a corresponding state of a plurality of states 320 associated with NSCLC. Disease indicator 318 may take various forms. In certain embodiments, disease indicator 318 is a score that indicates a classification of the corresponding state for biological sample 112. In certain embodiments, the classification of the corresponding state for biological sample 112 may be a binary classification (e.g., a prediction score of 0.8, 0.9, or otherwise approximately “1.0” may indicate an NSCLC state, while a prediction score of 0.1., 0.2., otherwise approximately “0.0” may indicate a healthy state). Thus, in accordance with the presently-disclosed embodiments, the disease indicator 318 may simply indicate whether a subject has NSCLC or does not have NSCLC based on the peptide structure data 310.

In certain embodiments, disease indicator 318 may include a score that indicates a probability that a subject (e.g., subject 114 in FIG. 1) falls within one of the states 320 associated with NSCLC. For example, disease indicator 318 may include one or more scores, each of which may indicate whether biological sample 112 has one of the corresponding states 320 associated with NSCLC. In some examples, disease indicator 318 may include a score for each of the states 320 associated with NSCLC. For example, as generally noted above, the states 320 associated with NSCLC may include a binary classification, in which, for example, the machine-learning systems 316 may generate prediction value of 0.8, 0.9, or otherwise approximately “1.0” to indicate the presence of NSCLC and may generate prediction value of 0.1, 0.2, otherwise approximately “0.0” to indicate the absence of NSCLC (e.g., a healthy). In certain embodiments, machine-learning systems 316 may include regularized regression model (e.g., a univariate regularized regression model or a multivariate regularized regression model). For example, in one embodiment, the regularized regression may include a LASSO regression model (e.g., a univariate LASSO regression model or a multivariate LASSO regression model) trained to compute disease indicator 318. In another embodiment, the regularized regression model may be trained to, for example, classify a biological sample obtained from a subject with respect to a plurality of states associated with NSCLC, detect the presence of one of a plurality of states associated with NSCLC; determine techniques for treating NSCLC in a subject; and determine techniques for diagnosing an individual with NSCLC, in accordance with the presently disclosed embodiments. The regularized regression model may be trained to identify weight coefficients for peptide structures of set of peptide structures 312. Peptide structure analyzer 308 may generate final output 128 based on disease indicator 318 that is output by model 314. In other embodiments, final output 128 may be an output generated by model 314.

In certain embodiments, final output 128 may include disease indicator 318. In other embodiments, final output 128 may include diagnosis output 324 and/or treatment output 326. Diagnosis output 324 may include, for example, an identification of a classification of which of the states 320 evidenced by biological sample 112 based on disease indicator 318. Treatment output 326 may include, for example, at least one of an identification of a therapy to treat the subject, a design for the therapy, or a treatment plan for administering the therapy. In certain embodiments, the therapy is a surgery, a chemotherapeutic therapy, a patient-specific therapy, a targeted immunotherapy, a radiation procedure, a radiofrequency ablation (RFA) procedure, or a combination thereof. Final output 128 may be sent to remote system 130 for processing in some examples. In other embodiments, final output 128 may be displayed on graphical user interface 328 in display system 306 for viewing by a human operator. The human operator may use final output 128 to diagnose and/or treat the subject when final output 128 indicates the subject has NSCLC.

### V.C. Analysis System for Peptide Structure Data Analysis for Diagnosis of Ovarian Cancer

FIG. 3 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning system 314 that comprises any number of or combination of the models or algorithms described above.

In various embodiments, model 312 analyzes peptide structure data 310 to generate disease indicator 316 that indicates whether the biological sample is positive for an ovarian cancer disease state based on set of peptide structures 318 identified as being associated with the ovarian cancer disease state. Peptide structure data 310 may include quantification data for the plurality of peptide structures. Quantification data for a peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to biological sample 112.

Disease indicator 316 may take various forms. In some examples, disease indicator 316 includes a classification that indicates whether or not the subject is positive for the ovarian cancer disease state. In various embodiments, disease indicator 316 can include a score 320. Score 320 indicates whether the ovarian cancer disease state is present or not. For example, score 320 may be, a probability score that indicates how likely it is that the biological sample 112 evidences the presence of the ovarian cancer disease state.

In one or more embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the ovarian cancer disease state based on training of model 312. In one or more embodiments, set of peptide structures 318 includes at least one, at least two, or at least three peptide structures from a first group of peptide structures (peptide structures PS-165 through PS-174) identified in Table 41. or at least one, at least two, or at least three peptide structures from a second group of peptide structures (peptide structures PS-169 and PS-175 through PS-198) identified in Table 42. For example, in one or more embodiments, set of peptide structures 318 includes at least 1, at least 2, at least 3, at least 4, at least 5, at least 6, at least 7, at least 8, at least 9, or all 10 of the peptide structures identified in Table 41. In one or more other embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures identified in Table 42. In one or more embodiments, set of peptide structures 318 includes at least peptide structure PS-169, which is identified in both Table 41 and Table 42. In some cases, the number of peptide structures selected from Table 41 for inclusion in set of peptide structures 318 may be based on, for example, a desired level of accuracy.

In one or more embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures identified in Table 43A. In one or more embodiments, set of peptide structures 318 includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, 49, 50, 51, 52, 53, 54, 55, 56, 57, 58, 59, 60, or all 61 of the peptide structures listed in Tables 41, 42, 43A, 43B, 43C, and 43D.

In various embodiments, machine learning system 314 takes the form of binary classification model 322. Binary classification model 322 may include, for example, but is not limited to, a regression model. Binary classification model 322 may include, for example, a penalized multivariable regression model that is trained to identify set of peptide structures 318 from a plurality of (or panel of) peptide structures identified in various subjects. Binary classification model 322 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 318.

Peptide structure analyzer 308 may generate final output 128 based on disease indicator 316 output by model 312. In other embodiments, final output 128 may be an output generated by model 312.

In some embodiments, final output 128 includes disease indicator 316. In one or more embodiments, final output 128 includes diagnosis output 324, treatment output 326, or both. Diagnosis output 324 may include, for example, a diagnosis for the ovarian cancer disease state. The diagnosis can include a positive diagnosis or a negative diagnosis for the ovarian cancer disease state. In one or more embodiments, generating diagnosis output 324 may include comparing score 320 to selected threshold 328 to determine the diagnosis. Selected threshold 328 may be, for example, without limitation, a score between 0.30 and 0.65 (e.g., 0.4, 0.5, 0.6, etc.). For example, when selected threshold 328 is set to 0.5, a score 320 above 0.5 (or at or above 0.5) may indicate the presence of the ovarian cancer disease state and be output in diagnosis output 324 as a positive diagnosis. A score 320 below 0.5 (or at or below 0.5) may indicate that the ovarian cancer disease state is not present and be output in diagnosis output 324 as a negative diagnosis. In one or more embodiments, a negative diagnosis indicates that the subject is healthy. In one or more embodiments, a negative diagnosis indicates that a detected pelvic tumor (or mass) is benign.

In one or more embodiments, when disease indicator 316 and/or diagnosis output 324 indicate a positive diagnosis for the ovarian cancer disease state, a biopsy may be recommended. For example, a biopsy of the subject may be performed in response to disease indicator 316 and/or diagnosis output 324 indicating a positive diagnosis for the ovarian cancer disease state. In some embodiments, peptide structure analyzer 308 (or another system implemented on computing platform 302) may generate a report recommending that a biopsy is to be performed for the subject in response to disease indicator 316 and/or diagnosis output 324 indicating a positive diagnosis for the ovarian cancer disease state. In other embodiments, peptide structure analyzer 308 may send diagnosis final output 128 to remote system 130 over one or more wireless, wired, and/or optical communications links and remote system 130 may generate a report recommending that a biopsy is to be performed for the subject in response to disease indicator 316 and/or diagnosis output 324 indicating a positive diagnosis for the ovarian cancer disease state. The biopsy may be used to confirm the diagnosis to determine whether or not to administer treatment and/or how quickly to administer treatment. When disease indicator 316 and/or diagnosis output 324 indicate a negative diagnosis for the ovarian cancer disease state (e.g., benign pelvic tumor), the report that is generated by peptide structure analyzer 308, remote system 130, or some other system implemented on computing platform 142 may recommend a period of monitoring for the subject. For example, a negative diagnosis indication by disease indicator 316 and/or diagnosis output 324 may thus help prevent unnecessary treatment or overtreatment of the subject.

Treatment output 326 may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof. Final output 128 may be sent to remote system 130 for processing in some examples. In other embodiments, final output 128 may be displayed on graphical user interface 330 in display system 306 for viewing by a human operator.

### V.D. Analysis System for Peptide Structure Data Analysis for Managing Melanoma Treatment

FIG. 3 is a block diagram of an analysis system 300 in accordance with one or more embodiments. Analysis system 300 can be used to both detect and analyze various peptide structures that have been associated with melanoma treatments. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform. In still other examples, computing platform 302 may include any number of or combination computers, cloud computing platforms, servers, or mobile devices.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, treatment management system 308, which may be implemented using hardware, software, firmware, or a combination thereof. In one or more embodiments, peptide structure analyzer 308 is implemented using computing platform 302.

Treatment management system 308 may be used to manage the treatment of a subject diagnosed with a melanoma condition (i.e., malignant melanoma). Treatment management system 308 may be used to predict the subject's response to one or more treatments for the melanoma condition, select a treatment to be administered to the subject to prevent the progression (or advancement) of the melanoma condition and/or otherwise improve the condition of the subject, and/or otherwise plan the treatment of the subject.

Treatment management system 308 receives peptide structure data 310 for processing. Peptide structure data 310 may have been generated using multiple reaction monitoring mass spectrometry. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112. Further, as set of peptide structures 122 corresponds to a set of glycoproteins (e.g., each peptide structure of set of peptide structures 122 being derived from a corresponding glycoprotein), peptide structure data 310 therefore corresponds to the set of glycoproteins. In some cases, two or more peptide structures may correspond to a same glycoprotein and these two or more peptide structures may be referred to as glycoforms of that same glycoprotein.

Peptide structure data 310 can be sent as input into treatment management system 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Treatment management system 308 may include scoring system 312. In one or more embodiments, treatment management system 308 further includes and treatment planning system 314. Scoring system 312 may be used to predict the response of a subject (e.g., subject 114) to one or more types of treatment. Treatment planning system 314 may be used to plan how to treat the subject based on the predicted response(s) for the subject.

Scoring system 312 may include, for example, model system 315 that is configured to receive peptide structure data 310 for processing. Model system 315 may be implemented in any of a number of different ways. Model system 315 may be a computational model system that may be implemented using any number of models, functions, equations, algorithms, and/or other mathematical techniques.

In one or more embodiments, scoring system 312 receives peptide structure data 310 for processing and inputs quantification data 316 identified from peptide structure data 310 for set of peptide structures 318 into model system 315. Model system 315 analyzes quantification data 316 to generate set of treatment scores 320 corresponding to a set of treatments. Peptide structure data 310 may comprise a set of quantification metrics for each peptide structure of, for example, set of peptide structures 122 in FIG. 1. A quantification metric for a peptide structure may be comprised of at least one of a relative abundance, a normalized abundance, an adjusted abundance, an absolute abundance, a relative quantity, an adjusted quantity, a normalized quantity, a relative concentration, an adjusted concentration, or a normalized concentration. Accordingly, quantification data 316 may include one or more quantification metrics for each peptide structure of set of peptide structures 318.

A peptide structure of set of peptide structures 318 may be a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. Alternatively, a peptide structure of set of peptide structures 318 may be an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the response of a subject to a corresponding treatment(s) based on training of model 312. In one or more embodiments, set of peptide structures 318 includes at least one, at least three, at least five, or at least some other number of the peptide structures identified in Table 55 below in Section V.B. The number of peptide structures selected from Table 55 for inclusion in set of peptide structures 318 may be based on, for example, a desired level of accuracy, the number of treatments for which set of treatment scores 320 are being generated, one or more other factors, or a combination thereof.

In one or more embodiments, model system 315 may be used to analyze the response of a subject to a pembrolizumab treatment (“pembro”), the response of the subject to a combination treatment comprised of the combination of nivolumab and ipilimumab (“ipi/nivo”). Both pembro and ipi/nivo are treatments used to treat melanoma. For example, model system 315 may use quantification data 316 for set of peptide structures 318 to generate set of treatment scores 320 that includes a first treatment score 322 for pembro and a second treatment score 324 for ipi/nivo. In one or more embodiments, set of peptide structures 318 may include first subset 321 of set of peptide structures 318 used to compute first treatment score 322 and second subset 323 of set of peptide structures 318 used to compute second treatment score 324. In one or more embodiments, first subset 321 and the second subset 323 of set of peptide structures 318 may partially overlap (e.g., have one, two, three, four, five, some other number of peptide structures in common.

First portion 326 of quantification data 316 used to compute first treatment score 322 may correspond to first subset 321. Second portion 328 of quantification data 316 used to compute second treatment score 324 may correspond to second subset 323. First portion 326 and second portion 328 may be referred to as first quantification data and second quantification data, respectively. When first subset 321 and second subset 323 partially overlap, first portion 326 and second portion 328 similarly overlap. As one example, first portion 326 of quantification data 316 corresponding to first portion 321 used to compute first treatment score 322 and second portion 328 of quantification data 316 corresponding to second subset 323 of set of peptide structures 318 used to compute second treatment score 324 may have two peptide structures in common.

In one or more embodiments, first subset 321 of set of peptide structures 318 includes at least one, at least three, at least five, or at least some other number of the peptide structures identified in Table 56 below in Section V.B. In one or more embodiments, second subset 323 of set of peptide structures 318 includes at least one, at least three, at least five, or at least some other number of the peptide structures identified in Table 57 below in Section V.B.

In one or more embodiments, set of peptide structures 318 may have been identified by treatment management system 308 using relevance system 330. Relevance system 330 may include any number of computational models to analyze sample data 332 to determine which peptide structures to include in set of peptide structures 318. Sample data 332 may be retrieved from data store 304 or received in some other manner. Sample data 332 may include data capturing multiple subjects' responses to one or more treatments. For example, sample data 332 may include data capturing subjects' responses to pembro and to subjects' responses to ipi/nivo.

In one or more embodiments, relevance system 330 includes a first algorithm that uses a Wilcoxon rank-sum test to determine first subset 321 and a second algorithm that uses the Wilcoxon rank-sum test to determine second subset 323. For example, relevance system 330 includes a first algorithm that uses a Wilcoxon rank-sum test to determine which peptide structures to include in first subset 321 to compute first treatment score 322 (e.g., for pembro) and a second algorithm that uses the Wilcoxon rank-sum test to determine which peptide structures to include in second subset 323 to compute second treatment score 324 (e.g., for ipi/nivo).

Treatment planning system 314 receives set of treatment scores 320 from scoring system 312. Treatment planning system 314 uses set of treatment scores 320 to generate treatment output 334. Treatment output 334 may include, for example, an identification or categorization of the response of the subject to the one or more treatments for which the subject's response is being predicted, at least one of an identification of a therapeutic to treat the subject, a design for the therapeutic, a treatment plan for administering the therapeutic, or a combination thereof. In some embodiments, the therapeutic is an immune checkpoint inhibitor. In various embodiments, treatment output 326 includes a therapeutic dosage for each therapeutic to be used in treating the subject.

In one or more embodiments, treatment output 334 identifies a response classification that indicates a predicted response for the subject to a treatment. For example, set of treatment scores 320 may include a treatment score that can be used to classify a subject's response to a melanoma treatment as either early disruption or sustained control.

The response classification may be, for example, a positive response classification, a negative response classification, or some other type of response classification. A positive response classification may, for example, indicate that the subject is predicted to have a relatively positive or otherwise successful response to treatment. A negative response classification may, for example, indicate that the subject is predicted to have a relatively poor or otherwise unsuccessful response to treatment. In one or more embodiments, the response classification predicts response to treatment with respect to survivability (e.g., overall survival, progression-free survival, etc.).

“Early disruption” may be an example of a negative response classification. “Early disruption” may indicate that the subject is predicted to have a relatively poor response to the treatment. For example, a prediction of “early disruption” may mean that the subject is predicted to have a disruption event within an initial period of time (e.g., 6 months) after treatment. A disruption event may be any event that disrupts the subject's “progression-free survival” (PFS). A disruption event may be also referred to as a progression event or an advancement event as such an event indicates disease progression or advancement. In some cases, the progression event may be a final level of progression or disease advancement, such as death. Thus, “early disruption” may also be referred to as “progression,” “disease progression,” or “disease advancement.” A disruption event may include, for example, at least one of a new melanoma (e.g., malignant mole), an increase in the size of an existing melanoma, or some other type of event. A disruption event may be detected using any number of progression criteria. For example, a disruption event may be considered “detected” in response to a selected number or proportion of a set of progression criteria being met. The set of progression criteria may include, for example, but is not limited to, one or more immune-related response criteria (irRC), one or more response evaluation criteria in solid tumors (RECIST), one or more other types of criteria, or a combination thereof.

“Sustained control” may be one example of a positive response classification. “Sustained control” may be a response classification that indicates that the subject is predicted to have a relatively successful response to the treatment. For example, a prediction of “sustained control” may mean that the subject is predicted to have no disruption events within a sustained period of time (e.g., 12 months) after treatment. The sustained period of time may be longer than the initial period of time.

In one or more embodiments, treatment planning system 314 uses one or more selected thresholds to classify set of treatment scores 320. In one or more embodiments, a different selected threshold is used for each treatment. In other embodiments, a same threshold is used for all treatments being considered. For example, treatment planning system 314 may use selected threshold 336. In one or more embodiments, selected threshold is 0.5. In other embodiments, selected threshold is 0.6, 0.7, 0.75, 0.8, or some other threshold.

As one example, when selected threshold is 0.5, treatment planning system 314 may generate a first predicted response based on a determination that a treatment score is above (or is at and above) the selected threshold and may generate a second predicted response based on a determination that the treatment score is not above (or is below) the selected threshold. The first predicted response may be, for example, a first predicted response classification (e.g., sustained control); the second predicted response may be a second predicted response classification (e.g., early disruption).

Treatment output 334 may include the response classification that is predicted such that a user (e.g., a medical professional) can determine whether a corresponding treatment should be or should not be administered to a subject. For example, when first treatment score 322 is generated for pembro, and treatment output 334 indicates that a subject's predicted response is “early disruption,” a medical professional may determine to administer a different treatment, a higher dosage of pembro, or change the treatment plan for the subject in some other way.

When set of treatment scores 320 includes at least two treatment scores, treatment planning system 314 may analyze the at least two treatment scores and determine which treatment score indicates a best response to the corresponding treatment for the subject. As one example, treatment planning system 314 may compare the at least treatment scores and select the treatment corresponding to the highest treatment score for the subject. This selected treatment may then be identified in treatment output 334. In some cases, treatment output 334 may further include a therapeutic dosage (e.g., an approved dosage) for selected treatment for the subject. In some cases, treatment output 334 may further include a response classification for the selected treatment. For example, while first treatment score 322 may be higher than second treatment score 324, both first treatment score 322 and second treatment score 324 may indicate that the predicted response for the subject is “early disruption” with both treatments. In this example, treatment output 336 may identify the treatment corresponding to first treatment score 322 with an indication that the predicted response “early disruption” and a recommendation to either select a different treatment, alter (e.g., increase/decrease) a dosage of the treatment corresponding to first treatment score 322, combine the treatment with at least one other treatment, or change the treatment plan for the subject in some other manner.

Treatment output 334 may be sent to remote system 130 for processing in some examples. In other embodiments, treatment output 334 may be displayed on graphical user interface 338 in display system 306 for viewing by a human operator. The human operator may use treatment output 334 to manage the melanoma treatment of the subject.

### V.E. Computer Implemented System

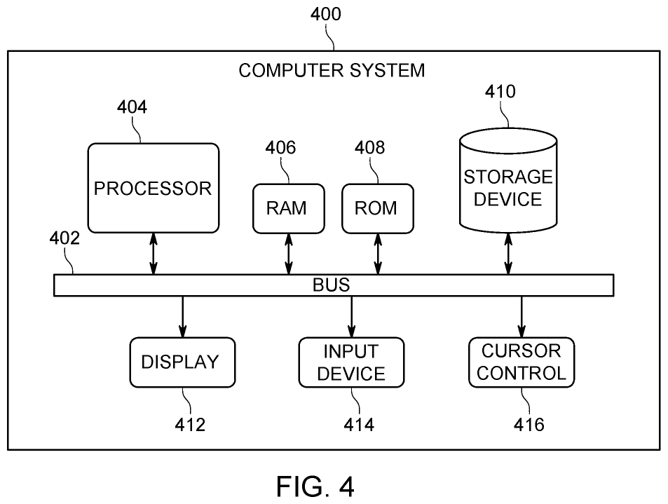

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of or combination of, the memory components RAM 406, ROM, 408, or storage device 410 and user input provided via input device 414.

### V.F. Computer Implemented System for Diagnosis of NSCLC

FIG. 4 illustrates a block diagram of a computer system that may be utilized for performing one or more methods of classifying a biological sample obtained from a subject with respect to a plurality of states associated with NSCLC, detecting the presence of one of a plurality of states associated with NSCLC, treating NSCLC in a subject, treating NSCLC in a subject, diagnosing an individual with NSCLC, and training a model to diagnose a subject with one of a plurality of states associated with NSCLC, in accordance with the presently disclosed embodiments. Computer system 400 may be an example of one implementation for computing platform 302 described above in FIG. 3. In certain embodiments, computer system 400 can include a bus 402 or other communication mechanism for communicating information, and a processor 404 coupled with bus 402 for processing information. In certain embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In certain embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

### V.G. Computer Implemented System for Diagnosis of Ovarian Cancer

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### V.H. Computer Implemented System for Managing Melanoma

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as R, C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### VI. Exemplary Methodologies Relating to Diagnosis based on Peptide Structure Data Analysis

### VIA. General Methodology

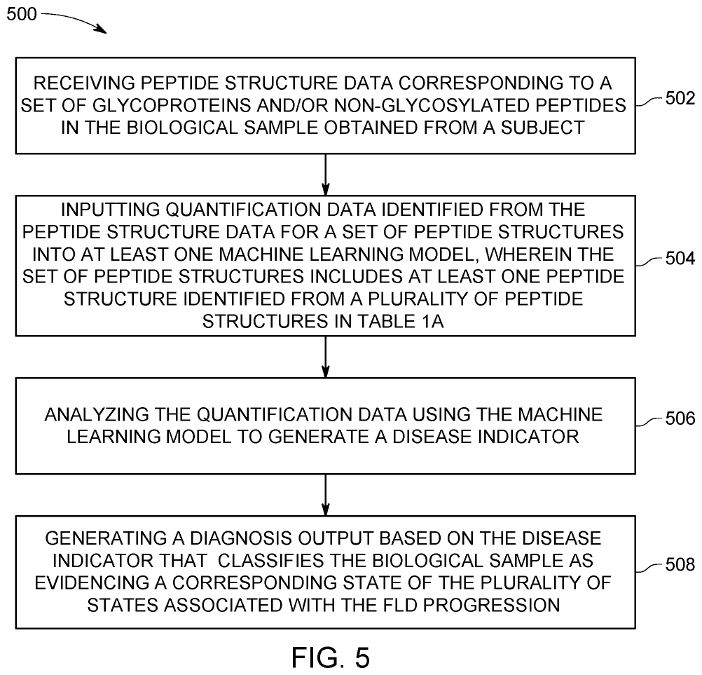

FIG. 5 is a flowchart of a process for classifying a biological sample obtained from a subject with respect to a plurality of states associated with fatty liver (FLD) progression. Process 500 may be implemented using, for example, at least a portion of workflow 100 as described in FIGS. 1, 2A, and 2B and/or analysis system 300 as described in FIG. 3. Process 500 may be used to generate a diagnosis output such as, for example, diagnosis output 324 in FIG. 3.

Step 502 includes receiving peptide structure data corresponding to a set of non-glycosylated peptides and/or glycopeptides in the biological sample obtained from a subject. The peptide structure data may be, for example, one example of an implementation of peptide structure data 310 in FIG. 3. The peptide structure data may include quantification data for each peptide structure of a plurality of peptide structures. In certain embodiments, the set of peptide structures includes at least one peptide structure identified from a plurality of peptide structures in Table 1A or 1B. The peptide structure data may have been generated using, for example, without limitation, multiple reaction monitoring mass spectrometry (MRM-MS). In various embodiments, the peptide structure data includes quantification data for the plurality of peptide structures. This quantification data for a peptide structure may include, for example, without limitation, at least one of an abundance, a relative abundance, a normalized abundance, a relative quantity, an adjusted quantity, a normalized quantity, a relative concentration, an adjusted concentration, or a normalized concentration for the peptide structure. In various embodiments, the quantification data comprises normalized abundances for the peptide structures.

In various embodiments, the peptide structure data is generated for a sample created from the biological sample, and in various embodiments the sample is a serum sample. For example, the biological sample may be prepared using reduction, alkylation, and enzymatic digestion to form a prepared sample. The prepared sample includes the plurality of peptide structures for which the peptide structure data is generated and then received in step 502.

Step 504 includes inputting quantification data identified from the peptide structure data for a set of peptide structures into a machine learning model. In some embodiments, the set of peptide structures includes at least one peptide structure identified from a plurality of peptide structures in Table 1A or 1B. In various embodiments, at least one peptide structure comprises a non-glycosylated peptide and/or a glycopeptide structure defined by a peptide sequence and a glycan structure linked to the peptide sequence at a linking site of the peptide sequence, as identified in Table 1A or 1B.

The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In various embodiments, the quantification data comprises normalized abundances for the peptide structures. The machine learning model may be, for example, a supervised machine learning model.

Step 506 includes analyzing the quantification data using the machine learning model to generate a disease indicator. In various embodiments, the disease indicator that is generated may include an indication of whether the biological sample evidences a state associated with FLD progression. For example, the disease indicator may indicate whether the biological sample is likely positive for NASH or not or may indicate a stage of NASH, such as early stage vs. late stage. In various embodiments, the disease indicator comprises a probability that the biological sample evidences a NASH state or a probability that the biological sample evidences an early NASH stage vs. late NASH stage. In various embodiments, the machine learning model may generate an output that classifies the biological sample as positive for the NASH state, positive for a healthy (or non-NASH) state, or that classifies the biological sample as being a stage of early NASH stage vs. late NASH stage. In various embodiments, the disease indicator can be a score.

Step 508 includes generating a diagnosis output based on the disease indicator that classifies the biological sample as evidencing a corresponding state of a plurality of states associated with the FLD progression. In various embodiments, the plurality of states can include a NASH state and a non-NASH state, or the plurality of states can include a stage of NASH, such as early stage or late stage. In some embodiments, the plurality of states can include a non-NASH state that comprises at least one of a healthy state, a liver disease-free state, or some other type of non-NASH state.

When the disease indicator is a score, step 508 may include determining that the score falls within a selected range associated with the corresponding state of the plurality of states. In various embodiments, for Model 1, a predicted probability >0.5 may indicate a likelihood of control or healthy, and a predicted probability <0.5 may indicate a likelihood of the presence of NASH, whereas for Model 2, a predicted probability >0.5 may indicate a likelihood of the presence of early stage NASH and <0.5 may indicate a likelihood of the presence of late stage NASH. In some embodiments, step 506 may include determining that the biological sample evidences the corresponding state based on a determination that the score falls within the selected range associated with corresponding state.

In various embodiments, generating the diagnosis output in step 508 includes generating the diagnosis output as part of a report that identifies the corresponding state. In some embodiments, step 508 may also include generating a treatment output based on at least one of the diagnosis output or the disease indicator. In some embodiments, the treatment output comprises at least one of an identification of a treatment to treat the subject, a design for the treatment, a manufacturing plan for the treatment, or a treatment plan for administering the treatment.

For a subject that has been diagnosed with NASH, the treatment may include, for example, a therapeutically effective dosage of at least one of Obeticholic acid (OCA), Tropifexor, Elafibranor, Saroglitazar, Aramchol, Semaglutide, Tirzepatide, Cotadutide, NGM282, MSDC-0602K, Resmetirom, Cenicriviroc, Selonsertib, Emricasan, Simtuzumab, and GR-MD-02. Additionally or alternatively, the subject may be recommended to lose weight, exercise more, control glucose level, reduce cholesterol, limit salt intake, limit sugar intake, reduce cholesterol, avoid or reduce alcohol intake, avoid liver-harming medications or dietary supplements, get vaccinated for hepatitis A, get vaccinated for hepatitis B, take vitamin E, take pioglitazone, take liraglutide, monitor for hepatic cell carcinoma (HCC), or a combination thereof.

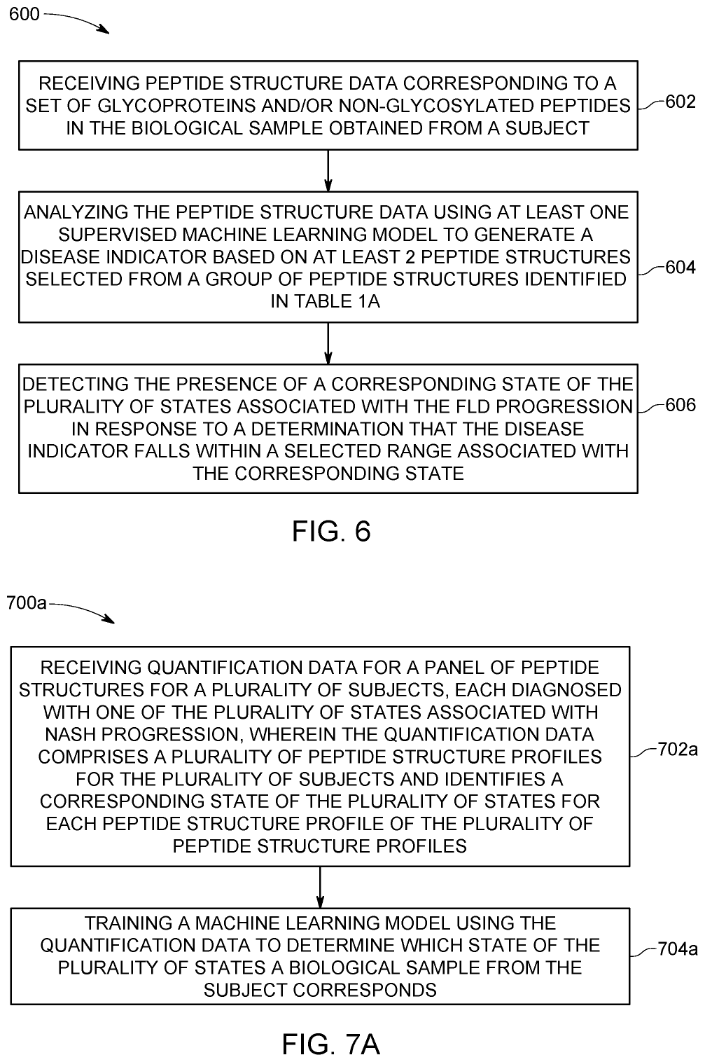

FIG. 6 is a flowchart of a process for detecting a presence of one of a plurality of states associated with fatty liver disease (FLD) progression in a biological sample. Process 600 may be implemented using, for example, at least a portion of workflow 100 as described in FIGS. 1, 2A, and 2B and/or analysis system 300 as described in FIG. 3.

Step 602 includes receiving peptide structure data corresponding to a set of non-glycosylated peptides and/or glycoproteins in the biological sample obtained from a subject. The peptide structure data may have been generated from a prepared sample using, for example, multiple reaction monitoring mass spectrometry (MRM-MS). The peptide structure data may include quantification data for each peptide structure of a panel of peptide structures. The quantification data for a peptide structure of the plurality of peptide structures may include at least one of a relative quantity, an adjusted quantity, a normalized quantity, a relative concentration, an adjusted concentration, a normalized concentration, or another quantification metric.

Step 604 includes analyzing the peptide structure data using a supervised machine learning model to generate a disease indicator based on at least 2 peptide structures selected from a group of peptide structures identified in Table 1A or 1B. In various embodiments, the supervised machine learning model comprises a logistic regression model.

In various embodiments, the at least 2 peptide structures include a non-glycosylated peptide and/or glycopeptide structure defined by a peptide sequence and a glycan structure linked to the peptide sequence at a linking site of the peptide sequence, as identified in Table 1A or 1B, with the peptide sequence being one of SEQ ID NOS: 1-23 as defined in Table 1A and SEQ ID NOS: 1-11 as defined in Table 1B, respectively.

In various embodiments, the at least 2 peptide structures include only peptide structures fragmented from alpha-1-antitrypsin (A1AT). In various embodiments, the at least 2 peptide structures include only peptide structures fragmented from alpha-2-macroglobulin (A2MG). In various embodiments, the at least 2 peptide structures include only peptide structures fragmented from Alpha-1-antichymotrypsin (AACT). In various embodiments, the at least 2 peptide structures include only peptide structures fragmented from Alpha-1-acid glycoprotein 1 and/or Alpha-1-acid glycoprotein 2 (AGP12). In various embodiments, the at least 2 peptide structures include only peptide structures fragmented from Apolipoprotein B-100 (APOB). In various embodiments, the at least 2 peptide structures include only peptide structures fragmented from Apolipoprotein C-III (APOC3). In various embodiments, the at least 2 peptide structures include only peptide structures fragmented from Histidine-rich Glycoprotein (HRG). In various embodiments, the at least 2 peptide structures include only peptide structures fragmented from Immunoglobulin heavy constant alpha 1 and/or 2 (IGA12). In various embodiments, the at least 2 peptide structures include only peptide structures fragmented from Immunoglobulin heavy constant gamma 1 (IGG1). In various embodiments, the at least 2 peptide structures include only peptide structures fragmented from Immunoglobulin heavy constant gamma 2 (IGG2). In various embodiments, the at least 2 peptide structures include only peptide structures fragmented from Plasma Kallikrein (KLKB1). In various embodiments, the at least 2 peptide structures include only peptide structures fragmented from Antithrombin-III.

In various embodiments, step 604 may include computing a peptide structure profile for the biological sample that identifies a weighted value for each peptide structure of the at least 2 peptide structures. In some embodiments, the weighted value for a peptide structure of the at least 2 peptide structures can be a product of a quantification metric for the peptide structure identified from the peptide structure data and a weight coefficient for the peptide structure. The weight coefficient of a corresponding peptide structure of the at least 2 peptide structures may indicate the relative significance of the corresponding peptide structure to the disease indicator. In various embodiments, analyzing the peptide structure data comprises computing the disease indicator using the peptide structure profile. The disease indicator may be, for example, a score that that indicates which state of a plurality of states is evidenced by the biological sample.

In various embodiments, the supervised machine learning (ML) model can employ, for example, one model or multiple different models for generating the final disease indicator. For example, the ML model can employ three different models, where each may each be, for example, a regression model that is used to analyze the peptide structure quantification data for a particular state versus the other states. For example, each of these three regression models may be used to distinguish between a given state of the plurality of states and the other states of the plurality of states.

Step 606 includes detecting the presence of a corresponding state of the plurality of states associated with the FLD progression in response to a determination that the disease indicator falls within a selected range associated with the corresponding state. In some embodiments, the plurality of states includes at least two selected from a group consisting of a non-alcoholic steatohepatitis (NASH) state, a non-NASH state (e.g., a control state, a healthy state, a liver disease-free state, etc.), and a stage of NASH, including early stage vs. late stage.

In various embodiments, the corresponding state is a non-NASH state and the selected range for the disease indicator associated with the non-NASH state may have a predicted probability >0.5. In some embodiments, the corresponding state is a NASH state and the selected range for the disease indicator associated with the NASH state may have a predicted probability <0.5. In some embodiments, the corresponding state is an early NASH stage and the selected range for the disease indicator associated with the early NASH stage may have a predicted probability >0.5. In some embodiments, the corresponding state is a late NASH stage and the selected range for the disease indicator associated with the late NASH stage may have a predicted probability <0.5.

In various embodiments, process 600 may further include generating a report that includes a diagnosis based on the corresponding state detected for the subject. The report may include, for example, the disease indicator.

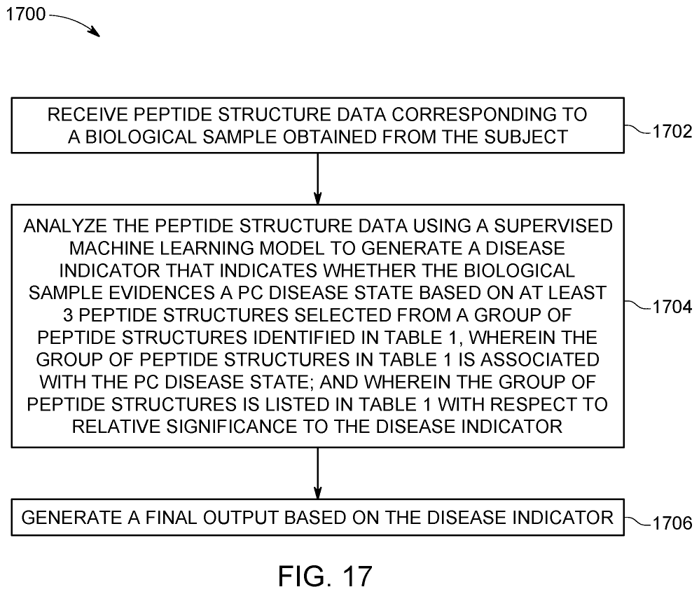

FIG. 17 is a flowchart of a process for diagnosing a subject with respect to a pancreatic cancer (PC) disease state in accordance with one or more embodiments. Process 500 may be implemented using, for example, at least a portion of workflow 100 as described in FIGS. 1, 2A, and 2B and/or analysis system 300 as described in FIG. 3. Process 1700 may be used to generate a final output that includes at least a diagnosis output for the subject.

Step 1702 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 16, with the peptide sequence being one of SEQ ID NOS: 77-119 as defined in Table 16.

Step 1704 includes analyzing the peptide structure data using a supervised machine learning model to generate a disease indicator that indicates whether the biological sample evidences a PC disease state based on at least 1 peptide structure selected from a group of peptide structures identified in Table 16, Table 17A, Table 17B, or Table 17C (below). In step 1704, the group of peptide structures in Table 16, Table 17A, Table 17B, or Table 17C is associated with the PC disease state. The group of peptide structures is listed in Table 16, Table 17A, Table 17B, or Table 17C with respect to relative significance to the disease indicator.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, or all 55 of the peptide structures PS-48 through PS-102 in Table 16.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures PS-49, PS-50, PS-54, PS-61, PS-63, PS-64, PS-71, PS-79, PS-81, PS-84, PS-86, PS-87, PS-90, PS-91, PS-92, PS-94, PS-95, PS-96, PS-97, PS-98, PS-99, or PS-101 in Table 17A.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or all 19 of the peptide structures PS-48, PS-52, PS-57, PS-61, PS-62, PS-63, PS-64, PS-69, PS-71, PS-72, PS-73, PS-84, PS-86, PS-88, PS-91, PS-94, PS-96, PS-100, or PS-101 in Table 17B.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, or all 17 of the peptide structures PS-48, PS-52, PS-61, PS-64, PS-66, PS-68, PS-69, PS-71, PS-72, PS-73, PS-86, PS-89, PS-91, PS-94, PS-96, PS-99, or PS-101 in Table 17C.

TABLE 16
Peptide Structures associated with Pancreatic Cancer

| | | | | Linking Site | Linking Site | | |
|---|---|---|---|---|---|---|---|
| | Peptide | Prot | Pept | Position | Position | Glycan | |
| PS- | Structure (PS)- | SEQ ID | SEQ ID | within Prot | within | Struct | Monoisotopic |
| ID NO. | NAME | NO. | NO. | Seq | Pept Seq | GL NO. | mass |
| 48 | A1AT_107_5412 | 120 | 77 | 107 | 14 | 5412 | 6041.6468 |
| 49 | A1AT_107_6512 | 120 | 77 | 107 | 14 | 6512 | 6406.779 |
| 50 | A1AT_107_NONGLYCOSYLATED | 120 | 77 | N/A | N/A | N/A | 3690.8165 |
| 51 | A1AT_70_5402 | 120 | 78 | 70 | 7 | 5402 | 5385.4001 |
| 52 | A1BG_179_5402 | 121 | 79 | 179 | 27 | 5402 | 6040.442 |
| 53 | A2MG_1424_NONGLYCOSYLATED | 122 | 80 | N/A | N/A | N/A | 2162.1735 |
| 54 | A2MG_247_5200 | 122 | 81 | 247 | 10 | 5200 | 4950.3109 |
| 55 | A2MG_247_5401 | 122 | 81 | 247 | 10 | 5401 | 5647.5651 |
| 56 | A2MG_247MC_5401 | 122 | 82 | 247 | 10 | 5401 | 4344.8784 |
| 57 | A2MG_55_5412 | 122 | 83 | 55 | 9 | 5412 | 4747.056 |
| 58 | A2MG_869_5401 | 122 | 84 | 869 | 6 | 5401 | 5326.2974 |
| 59 | A2MG_869_5402 | 122 | 84 | 869 | 6 | 5402 | 5617.3928 |
| 60 | A2MG_869_6301 | 122 | 84 | 869 | 6 | 6301 | 5285.2709 |
| 61 | AACT_271_6512 | 123 | 85 | 271 | 4 | 6512 | 4467.8355 |
| 62 | AGP1_93_7614 | 124 | 86 | 93 | 7 | 7614 | 5578.1749 |
| 63 | AGP12_56_5412 | 125 & 126 | 87 | 56 | 5 | 5412 | 3146.1702 |
| 64 | AGP12_72_7601 | 125 & 126 | 88 | 72 | 15 | 7601 | 4562.8878 |
| 65 | AGP12_72MC_6503 | 125 & 126 | 89 | 72 | 15 | 6503 | 5755.449 |
| 66 | APOB_3895_5401 | 127 | 90 | 3895 | 9 | 5401 | 3826.5976 |
| 67 | APOB_983_5402 | 127 | 91 | 983 | 16 | 5402 | 5754.3354 |
| 68 | APOH_253_5412 | 128 | 92 | 253 | 3 | 5412 | 3600.3886 |
| 69 | APOM_135_5401 | 129 | 93 | 135 | 15 | 5401 | 4444.819 |
| 70 | APOM_135_5402 | 129 | 93 | 135 | 15 | 5402 | 4735.9144 |
| 71 | C1S_174_5402 | 130 | 94 | 174 | 5 | 5402 | 5730.4016 |
| 72 | CERU_397_5412 | 131 | 95 | 397 | 2 | 5412 | 4476.8219 |
| 73 | CO5_741_5401 | 132 | 96 | 74 | 2 | 5401 | 2582.0375 |
| 74 | CO5_741_5402 | 132 | 96 | 741 | 2 | 5402 | 2873.1329 |
| 75 | FETUA_156_5412 | 133 | 97 | 156 | 12 | 5412 | 4121.6695 |
| 76 | HEMO_187_5412 | 134 | 98 | 187 | 7 | 5412 | 3754.4918 |
| 77 | HEMO_453_5401 | 134 | 99 | 453 | 7 | 5401 | 3648.5492 |
| 78 | HEMO_64_5402 | 134 | 100 | 64 | 15 | 5402 | 4731.8395 |
| 79 | HPT_207_121015 | 135 | 101 | 207, 211 | 5, 9 | 6502 & 6513 | 7034.6887 |
| 80 | HRG_125_5402 | 136 | 102 | 125 | 5 | 5402 | 4218.7401 |
| 81 | IC1_238_5412 | 137 | 103 | 238 | 6 | 5412 | 3259.2655 |
| 82 | IC1_253_5402 | 137 | 104 | 253 | 4 | 5402 | 4304.8575 |
| 83 | IC1_253_5412 | 137 | 104 | 253 | 4 | 5412 | 4450.9154 |
| 84 | IC1_253_6503 | 137 | 104 | 253 | 4 | 6503 | 4961.0851 |
| 85 | IC1_253_6513 | 137 | 104 | 253 | 4 | 6513 | 5107.143 |
| 86 | IC1_352_5402 | 137 | 105 | 352 | 9 | 5402 | 4517.1303 |
| 87 | IGA12_144_3500 | 138 & 139 | 106 | 144 (P01876) or | 18 | 3500 | 4464.1462 |
| | | | | 131 (P01877) | | | |
| 88 | IGG1_297_3410 | 140 | 107 | 180 | 5 | 3410 | 2633.0385 |
| 89 | IGG1_297_3500 | 140 | 107 | 180 | 5 | 3500 | 2690.06 |
| 90 | IGG1_297_3510 | 140 | 107 | 180 | 5 | 3510 | 2836.1179 |
| 91 | IGG2_297_3500 | 141 | 108 | 176 | 5 | 3500 | 2658.0702 |
| 92 | IGM_439_9200 | 142 | 109 | 440 | 9 | 9200 | 4228.7318 |
| 93 | KLKB1_494_6503 | 143 | 110 | 494 | 6 | 6503 | 5107.1769 |
| 94 | QUANTPEP- | 144 | 111 | N/A | N/A | N/A | 1178.6659 |
| | A2GL_DLLLPQPDLR | | | | | | |
| 95 | QUANTPEP- | 145 | 112 | N/A | N/A | N/A | 1234.6809 |
| | APOA1_DLATVYVDVLK | | | | | | |
| 96 | QUANTPEP- | 146 | 113 | N/A | N/A | N/A | 816.48576 |
| | APOM_AFLLTPR | | | | | | |
| 97 | QUANTPEP- | 147 | 114 | N/A | N/A | N/A | 1121.6193 |
| | B2M_VNHVTLSQPK | | | | | | |
| 98 | QUANTPEP- | 148 | 115 | N/A | N/A | N/A | 1542.7566 |
| | FINC_SYTITGLQPGTDYK | | | | | | |
| 99 | QUANTPEP- | 149 | 116 | N/A | N/A | N/A | 2454.1438 |
| | TTR_TSESGELHGLTTEEEFVEGIYK | | | | | | |
| 100 | SHBG_380_5402 | 150 | 117 | 380 | 8 | 5402 | 3247.3131 |
| 101 | TRFE_432_5401 | 151 | 118 | 432 | 12 | 5401 | 3389.4212 |
| 102 | VTNC_169_5401 | 152 | 119 | 169 | 1 | 5401 | 2824.1431 |

TABLE 17A
Peptide Structures After LASSO Shrinkage; Model 1: Healthy vs.
Pancreatic Cancer

| | | (Protein) | (Peptide) |
|---|---|---|---|
| PS-ID | | SEQ ID | SEQ ID |
| NO. | PS-NAME | NO. | NO. |
| 86 | IC1_352_5402 | 137 | 105 |
| 96 | QUANTPEP-APOM_AFLLTPR | 146 | 113 |
| 84 | IC1_253_6503 | 137 | 104 |
| 101 | TRFE_432_5401 | 151 | 118 |
| 91 | IGG2_297_3500 | 141 | 108 |
| 71 | C1S_174_5402 | 130 | 94 |
| 94 | QUANTPEP-A2GL_DLLLPQPDLR | 144 | 111 |
| 97 | QUANTPEP-B2M_VNHVTLSQPK | 147 | 114 |
| 54 | A2MG_247_5200 | 122 | 81 |
| 79 | HPT_207_121015 | 135 | 101 |
| 81 | IC1_238_5412 | 137 | 103 |
| 99 | QUANTPEP- | 149 | 116 |
| | TTR_TSESGELHGLTTEEEFVEGIYK | | |
| 61 | AACT_271_6512 | 123 | 85 |
| 49 | A1AT_107_6512 | 120 | 77 |
| 95 | QUANTPEP- | 145 | 112 |
| | APOA1_DLATVYVDVLK | | |
| 50 | A1AT_107_NONGLYCOSYLATED | 120 | 77 |
| 98 | QUANTPEP- | 148 | 115 |
| | FINC_SYTITGLQPGTDYK | | |
| 92 | IGM_439_9200 | 142 | 109 |
| 90 | IGG1_297_3510 | 140 | 107 |
| 87 | IGA12_144_3500 | 138&139 | 106 |
| 64 | AGP12_72_7601 | 125&126 | 88 |
| 63 | AGP12_56_5412 | 125&126 | 87 |

TABLE 17B
Peptide Structures After LASSO Shrinkage; Model 2: Healthy/Benign
Pancreatitis vs. Pancreatic Cancer

| | | (Protein) | (Peptide) |
|---|---|---|---|
| PS-ID | | SEQ ID | SEQ ID |
| NO. | PS-NAME | NO. | NO. |
| 86 | IC1_352_5402 | 137 | 105 |
| 96 | QUANTPEP-APOM_AFLLTPR | 146 | 113 |
| 101 | TRFE_432_5401 | 151 | 118 |
| 69 | APOM_135_5401 | 129 | 93 |
| 73 | CO5_741_5401 | 132 | 96 |
| 48 | A1AT_107_5412 | 120 | 77 |
| 84 | IC1_253_6503 | 137 | 104 |
| 91 | IGG2_297_3500 | 141 | 108 |
| 64 | AGP12_72_7601 | 125&126 | 88 |
| 61 | AACT_271_6512 | 123 | 85 |
| 72 | CERU_397_5412 | 131 | 95 |
| 57 | A2MG_55_5412 | 122 | 83 |
| 62 | AGP1_93_7614 | 124 | 86 |
| 52 | A1BG_179_5402 | 121 | 79 |
| 100 | SHBG_380_5402 | 150 | 117 |
| 71 | C1S_174_5402 | 130 | 94 |
| 88 | IGG1_297_3410 | 140 | 107 |
| 94 | QUANTPEP-A2GL_DLLLPQPDLR | 144 | 111 |
| 63 | AGP12_56_5412 | 125&126 | 87 |

TABLE 17C
Peptide Structures After LASSO Shrinkage; Model 3: Healthy/Benign
Pancreatitis vs. Early Stage Pancreatic Cancer

| | | (Protein) | (Peptide) |
|---|---|---|---|
| PS-ID | | SEQ ID | SEQ ID |
| NO. | PS NAME | NO. | NO. |
| 96 | QUANTPEP-APOM_AFLLTPR | 146 | 113 |
| 86 | IC1_352_5402 | 137 | 105 |
| 101 | TRFE_432_5401 | 151 | 118 |
| 52 | A1BG_179_5402 | 121 | 79 |
| 69 | APOM_135_5401 | 129 | 93 |
| 91 | IGG2_297_3500 | 141 | 108 |
| 71 | C1S_174_5402 | 130 | 94 |
| 94 | QUANTPEP-A2GL_DLLLPQPDLR | 144 | 111 |
| 48 | A1AT_107_5412 | 120 | 77 |
| 73 | CO5_741_5401 | 132 | 96 |
| 64 | AGP12_72_7601 | 125&126 | 88 |
| 61 | AACT_271_6512 | 123 | 85 |
| 68 | APOH_253_5412 | 128 | 92 |
| 66 | APOB_3895_5401 | 127 | 90 |
| 99 | QUANTPEP- | 149 | 116 |
| | TTR_TSESGELHGLTTEEEFVEGIYK | | |
| 72 | CERU_397_5412 | 131 | 95 |
| 89 | IGG1_297_3500 | 140 | 107 |

In one or more embodiments, step 1704 may be implemented using a binary classification model (e.g., a regression model). In some examples, the regression model may be, for example, penalized multivariable regression model. In various embodiments, the disease indicator may be computed using a weight coefficient associated with each peptide structure of the at least 1 peptide structure, the weight coefficient of a corresponding peptide structure of the at least 1 peptide structure may indicate the relative significance of the corresponding peptide structure to the disease indicator.

In some embodiments, step 1704 may include computing a peptide structure profile for the biological sample that identifies a weighted value for each peptide structure of the at least 1 peptide structure. The weighted value for a peptide structure of the at least 1 peptide structure may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is positive for the PC disease state and the supervised machine learning model is configured to generate an output that identifies the biological sample as either evidencing (“positive for”) the PC disease state when the disease indicator is greater than a selected threshold or not evidencing (“negative for”) the PC disease state when the disease indicator is not greater than the selected threshold. The selected threshold may be, for example, 0.30, 0.35, 0.40, 0.45, 0.50, 0.55, or some other threshold. In one or more embodiments, the selected threshold is 0.5.

Step 506 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the PC disease state if the biological sample evidences the PC disease state based on the disease indicator. The diagnosis may be, for example, “negative” if the biological sample does not evidence the PC disease state based on the disease indicator. A negative diagnosis may mean that the biological sample has a non-pancreatic cancer (PC) state (e.g., healthy, control, etc.). The negative diagnosis for the PC disease state can include at least one of a healthy state, a benign pancreatitis state, or a control state.

Generating the diagnosis output in step 1706 may include determining that the score falls above a selected threshold and generating a positive diagnosis for the PC disease state. Alternatively, step 1706 can include determining that the score falls below a selected threshold and generating a negative diagnosis for the PC disease state. In some scoring systems, the score can include a probability score and the selected threshold can be 0.5. In other scoring systems, the selected threshold can fall within a range between 0.4 and 0.6.

In one or more embodiments, the final output in step 1706 may include a treatment output if the diagnosis output indicates a positive diagnosis for the PC disease state. The treatment output may include, for example, at least one of an identification of a treatment for the subject, a treatment plan for administering the treatment, or both. Treatment for pancreatic cancer may include, for example, but is not limited to, at least one of radiation therapy, chemoradiotherapy, surgery, a targeted drug therapy, or some other form of treatment. The treatment plan may include, for example, but is not limited to, a timeline or schedule for administering the treatment, dosing information, other treatment-related information, or a combination thereof.

### VI.B. General Methodology System for Diagnosis of NSCLC

In some embodiments, the methods provided herein are useful for diagnosing NSCLC. In some embodiments, the method is useful for diagnosing early-stage NSCLC. In some embodiments, the method is useful for diagnosing late-stage NSCLC. In some embodiments, the method comprises diagnosing an individual with NSCLC, wherein the individual has stage 0, stage I, stage II, stage III, or stage IV NSCLC. In some embodiments, the method comprises diagnosing an individual with NSCLC before the onset of disease symptoms and pathological conditions of NSCLC. In some embodiments, the method comprises diagnosing an individual with NSCLC after the onset of disease symptoms and pathological conditions of NSCLC. In some embodiments, the method comprises diagnosing an individual with NSCLC during disease symptoms and pathological conditions of NSCLC. In some embodiments, the method comprises diagnosing an individual with NSCLC before disease progression. In some embodiments, the method comprising diagnosing an individual with NSCLC before metastasis.

In some embodiments, the method comprises classifying a biological sample with respect to a plurality of states associated with NSCLC based upon one or more peptide structures provided in Table 35 and/or Table 40. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the glycopeptide structure data comprises one or more glycopeptide structure provided in Table 35 and/or Table 40. In some embodiments, the presence, absence, and/or amount of one or more peptides and/or glycopeptide is determined by MRM-MS. In some embodiments, the method comprises classifying a biological sample with respect to a plurality of states associated with NSCLC, based upon one or more peptide structure provided in Table 35 or Table 40, and selecting a treatment for NSCLC based upon the classification. In some embodiments, the method comprises classifying a biological sample with respect to a plurality of states associated with NSCLC, based upon one or more peptide structure provided in Table 35 or Table 40, and administering a treatment for NSCLC based upon the classification. In some embodiments, the method comprises classifying a biological sample with respect to a plurality of states associated with NSCLC, based upon one or more glycopeptide structure provided in Table 35 or Table 40, and administering a treatment for NSCLC based upon the classification.

In some embodiments, the diagnosis is based upon presence and/or amount of at least one, at least two, at least three, at least four, at least five, at least ten, at least 15, at least 20, or at least 25 peptide structures from Table 35. In some embodiments, the diagnosis is based upon presence and/or amount of at least one, at least two, at least three, at least four, at least five, at least ten, at least 15, at least 20, or at least 25 glycopeptides from Table 35. In some embodiments, the diagnosis is based upon the presence and/or amount of one or more peptides and/or glycopeptides comprising the amino acid sequence of SEQ ID NOs: 224-257. In some embodiments, the diagnosis is based upon the presence and/or amount of one or more glycopeptides comprising the amino acid sequence of SEQ ID NOs: 224-257. In some embodiments, the diagnosis is based upon the presence and/or amount of one or more peptides and/or glycopeptide comprising the amino acid sequence of SEQ ID NOs: 224-257 set forth in Table 35. In some embodiments, the diagnosis is based upon the presence and/or amount of one or more glycopeptides comprising the amino acid sequence of SEQ ID NOs: 224-257 along with the associated glycan set forth in Table 35. In some embodiments, the diagnosis is based upon the presence and/or amount of two or more peptides and/or glycopeptides comprising the amino acid sequence of SEQ ID NOs: 224-257 along with the associated glycan set forth in Table 35. In some embodiments, the diagnosis is based upon the presence and/or amount of three or more peptides and/or glycopeptides comprising the amino acid sequence of SEQ ID NOs: 224-257 along with the associated glycan set forth in Table 35. In some embodiments, the diagnosis is based upon the presence and/or amount of four or more peptides and/or glycopeptides comprising the amino acid sequence of SEQ ID NOs: 224-257 along with the associated glycan set forth in Table 35. In some embodiments, the diagnosis is based upon the presence and/or amount of five or more peptides and/or glycopeptides comprising the amino acid sequence of SEQ ID NOs: 224-257 along with the associated glycan set forth in Table 35. In some embodiments, the diagnosis is based upon the presence and/or amount of 10 or more peptides and/or glycopeptides comprising the amino acid sequence of SEQ ID NOs: 224-257 along with the associated glycan set forth in Table 35. In some embodiments, the diagnosis is based upon the presence and/or amount of 15 or more peptides and/or glycopeptides comprising the amino acid sequence of SEQ ID NOs: 224-257 along with the associated glycan set forth in Table 35. In some embodiments, the diagnosis is based upon the presence and/or amount of 20 or more peptides and/or glycopeptides comprising the amino acid sequence of SEQ ID NOs: 224-257 along with the associated glycan set forth in Table 35. In some embodiments, the diagnosis is based upon the presence and/or amount of 25 or more peptides and/or glycopeptides comprising the amino acid sequence of SEQ ID NOs: 224-257 along with the associated glycan set forth in Table 35. In some embodiments, the diagnosis is based upon the presence and/or amount of each of the peptides and/or glycopeptides comprising the amino acid sequence of SEQ ID NOs: 224-257 along with the associated glycan set forth in Table 35. In some embodiments, the method comprises classifying a biological sample with respect to a plurality of states associated with NSCLC based upon one or more peptide structure provided in Table 35 and selecting a treatment for NSCLC based upon the classification. In some embodiments, the method comprises classifying a biological sample with respect to a plurality of states associated with NSCLC based upon one or more peptide structure provided in Table 35 and administering a treatment for NSCLC based upon the classification.

In some embodiments, the diagnosis is based upon the presence and/or amount of one or more peptides and/or glycopeptides consisting of the amino acid sequence of SEQ ID NOs: 224-257 set forth in Table 35. In some embodiments, the diagnosis is based upon the presence and/or amount of one or more glycopeptides consisting of the amino acid sequence of SEQ ID NOs: 224-257 set forth in Table 35. In some embodiments, the diagnosis is based upon the presence and/or amount of one or more glycopeptides consisting of the amino acid sequence of SEQ ID NOs: 224-257 along with the associated glycan set forth in Table 35. In some embodiments, the diagnosis is based upon the presence and/or amount of two or more peptides and/or glycopeptides consisting of the amino acid sequence of SEQ ID NOs: 224-257 along with the associated glycan set forth in Table 35. In some embodiments, the diagnosis is based upon the presence and/or amount of three or more peptides and/or glycopeptides consisting of the amino acid sequence of SEQ ID NOs: 224-257 along with the associated glycan set forth in Table 35. In some embodiments, the diagnosis is based upon the presence and/or amount of four or more peptides and/or glycopeptides consisting of the amino acid sequence of SEQ ID NOs: 224-257 along with the associated glycan set forth in Table 35. In some embodiments, the diagnosis is based upon the presence and/or amount of five or more peptides and/or glycopeptides consisting of the amino acid sequence of SEQ ID NOs: 224-257 along with the associated glycan set forth in Table 35. In some embodiments, the diagnosis is based upon the presence and/or amount of 10 or more peptides and/or glycopeptides consisting of the amino acid sequence of SEQ ID NOs: 224-257 along with the associated glycan set forth in Table 35. In some embodiments, the diagnosis is based upon the presence and/or amount of 15 or more peptides and/or glycopeptides consisting of the amino acid sequence of SEQ ID NOs: 224-257 along with the associated glycan set forth in Table 35. In some embodiments, the diagnosis is based upon the presence and/or amount of 20 or more peptides and/or glycopeptides consisting of the amino acid sequence of SEQ ID NOs: 224-257 along with the associated glycan set forth in Table 35. In some embodiments, the diagnosis is based upon the presence and/or amount of 25 or more peptides and/or glycopeptides consisting of the amino acid sequence of SEQ ID NOs: 224-257 along with the associated glycan set forth in Table 35. In some embodiments, the diagnosis is based upon the presence and/or amount of each of the peptides and/or glycopeptides consisting of the amino acid sequence of SEQ ID NOs: 224-257 along with the associated glycan set forth in Table 35. In some embodiments, the method comprises classifying a biological sample with respect to a plurality of states associated with NSCLC based upon one or more peptide and/or glycopeptide structure provided in Table 35 and selecting a treatment for NSCLC based upon the classification. In some embodiments, the method comprises classifying a biological sample with respect to a plurality of states associated with NSCLC based upon one or more peptide and/or glycopeptide structure provided in Table 35 and administering a treatment for NSCLC based upon the classification.

In some embodiments, the method comprises determining whether an individual has NSCLC by inputting quantification data from peptide and/or glycopeptide structure data for a set of peptide structures comprising one or more, two or more, three or more, four or more, five or more, 10 or more, 15 or more, 20 or more, 25 or more, or each of the peptides and/or glycopeptide consisting of the amino acid sequence of SEQ ID NOs: 224-257 into a machine-learning model trained to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the machine-learning model is a regularized regression model. In some embodiments, the regularized model comprises a LASSO regression model. In some embodiments, the peptide structures are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein.

In some embodiments, the diagnosis is based upon presence and/or amount of at least one, at least two, at least three, at least four, at least five, at least 10, at least 15, at least 20, or at least 25 peptide structures from Table 35. In some embodiments, the diagnosis is based upon the presence and/or amount of one or more peptides and/or glycopeptides comprising the amino acid sequence of SEQ ID NOs: 258-296. In some embodiments, the diagnosis is based upon the presence and/or amount of one or more glycopeptides comprising the amino acid sequence of SEQ ID NOs: 258-296. In some embodiments, the diagnosis is based upon the presence and/or amount of one or more peptides and/or glycopeptide comprising the amino acid sequence of SEQ ID NOs: 258-296 set forth in Table 35. In some embodiments, the diagnosis is based upon the presence and/or amount of one or more glycopeptide comprising the amino acid sequence of SEQ ID NOs: 258-296 set forth in Table 35. In some embodiments, the diagnosis is based upon the presence and/or amount of one or more glycopeptides comprising the amino acid sequence of SEQ ID NOs: 258-296 along with the associated glycan set forth in Table 35. In some embodiments, the diagnosis is based upon the presence and/or amount of two or more peptides and/or glycopeptides comprising the amino acid sequence of SEQ ID NOs: 258-296 along with the associated glycan set forth in Table 35. In some embodiments, the diagnosis is based upon the presence and/or amount of three or more peptides and/or glycopeptides comprising the amino acid sequence of SEQ ID NOs: 258-296 along with the associated glycan set forth in Table 35. In some embodiments, the diagnosis is based upon the presence and/or amount of four or more peptides and/or glycopeptides comprising the amino acid sequence of SEQ ID NOs: 258-296 along with the associated glycan set forth in Table 35. In some embodiments, the diagnosis is based upon the presence and/or amount of five or more peptides and/or glycopeptides comprising the amino acid sequence of SEQ ID NOs: 258-296 along with the associated glycan set forth in Table 35. In some embodiments, the diagnosis is based upon the presence and/or amount of 10 or more peptides and/or glycopeptides comprising the amino acid sequence of SEQ ID NOs: 258-296 along with the associated glycan set forth in Table 35. In some embodiments, the diagnosis is based upon the presence and/or amount of 15 or more peptides and/or glycopeptides comprising the amino acid sequence of SEQ ID NOs: 258-296 along with the associated glycan set forth in Table 35. In some embodiments, the diagnosis is based upon the presence and/or amount of 20 or more peptides and/or glycopeptides comprising the amino acid sequence of SEQ ID NOs: 258-296 along with the associated glycan set forth in Table 35. In some embodiments, the diagnosis is based upon the presence and/or amount of 25 or more peptides and/or glycopeptides comprising the amino acid sequence of SEQ ID NOs: 258-296 along with the associated glycan set forth in Table 35. In some embodiments, the diagnosis is based upon the presence and/or amount of each of the peptides and/or glycopeptides comprising the amino acid sequence of SEQ ID NOs: 258-296 along with the associated glycan set forth in Table 35. In some embodiments, the method comprises classifying a biological sample with respect to a plurality of states associated with NSCLC based upon one or more peptide and/or glycopeptide structure provided in Table 35 and selecting a treatment for NSCLC based upon the classification. In some embodiments, the method comprises classifying a biological sample with respect to a plurality of states associated with NSCLC based upon one or more peptide and/or glycopeptide structure provided in Table 35 and administering a treatment for NSCLC based upon the classification.

In some embodiments, the diagnosis is based upon the presence and/or amount of one or more peptides and/or glycopeptides consisting of the amino acid sequence of SEQ ID NOs: 258-296 set forth in Table 35. In some embodiments, the diagnosis is based upon the presence and/or amount of one or more glycopeptides consisting of the amino acid sequence of SEQ ID NOs: 258-296 set forth in Table 35. In some embodiments, the diagnosis is based upon the presence and/or amount of one or more glycopeptides consisting of the amino acid sequence of SEQ ID NOs: 258-296 along with the associated glycan set forth in Table 35. In some embodiments, the diagnosis is based upon the presence and/or amount of two or more peptides and/or glycopeptides consisting of the amino acid sequence of SEQ ID NOs: 258-296 along with the associated glycan set forth in Table 35. In some embodiments, the diagnosis is based upon the presence and/or amount of three or more peptides and/or glycopeptides consisting of the amino acid sequence of SEQ ID NOs: 258-296 along with the associated glycan set forth in Table 35. In some embodiments, the diagnosis is based upon the presence and/or amount of four or more peptides and/or glycopeptides consisting of the amino acid sequence of SEQ ID NOs: 258-296 along with the associated glycan set forth in Table 35. In some embodiments, the diagnosis is based upon the presence and/or amount of five or more peptides and/or glycopeptides consisting of the amino acid sequence of SEQ ID NOs: 258-296 along with the associated glycan set forth in Table 35. In some embodiments, the diagnosis is based upon the presence and/or amount of 10 or more peptides and/or glycopeptides consisting of the amino acid sequence of SEQ ID NOs: 258-296 along with the associated glycan set forth in Table 35. In some embodiments, the diagnosis is based upon the presence and/or amount of 15 or more peptides and/or glycopeptides consisting of the amino acid sequence of SEQ ID NOs: 258-296 along with the associated glycan set forth in Table 35. In some embodiments, the diagnosis is based upon the presence and/or amount of 20 or more peptides and/or glycopeptides consisting of the amino acid sequence of SEQ ID NOs: 258-296 along with the associated glycan set forth in Table 35. In some embodiments, the diagnosis is based upon the presence and/or amount of 25 or more peptides and/or glycopeptides consisting of the amino acid sequence of SEQ ID NOs: 258-296 along with the associated glycan set forth in Table 35. In some embodiments, the diagnosis is based upon the presence and/or amount of each of the peptides and/or glycopeptides consisting of the amino acid sequence of SEQ ID NOs: 258-296 along with the associated glycan set forth in Table 35. In some embodiments, the method comprises classifying a biological sample with respect to a plurality of states associated with NSCLC based upon one or more peptide and/or glycopeptide structure provided in Table 35 and selecting a treatment for NSCLC based upon the classification. In some embodiments, the method comprises classifying a biological sample with respect to a plurality of states associated with NSCLC based upon one or more peptide and/or glycopeptide structure provided in Table 35 and administering a treatment for NSCLC based upon the classification.

In some embodiments, the method comprises determining whether an individual has NSCLC or is likely to develop NSCLC by inputting quantification data from peptide and/or glycopeptide structure data for a set of peptide structures comprising one or more, two or more, three or more, four or more, five or more, 10 or more, 15 or more, 20 or more, 25 or more, or each of the peptides and/or glycopeptides consisting of the amino acid sequence of SEQ ID NOs: 258-296 into a machine-learning model trained to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the machine-learning model is a regularized regression model. In some embodiments, the regularized regression model comprises a LASSO regression model. In some embodiments, the peptide structures are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein.

In some embodiments, the diagnosis is based upon presence and/or amount of at least one, at least two, at least three, at least four, at least five, at least 10, or at least 15 peptide structures from Table 40. In some embodiments, the diagnosis is based upon presence and/or amount of at least one, at least two, at least three, at least four, at least five, at least 10, or at least 15 glycopeptides from Table 40. In some embodiments, the diagnosis is based upon the presence and/or amount of one or more peptides and/or glycopeptides comprising the amino acid sequence of SEQ ID NOs: 229, 231-234, 236, 239, 241, 244-245, 247-255. In some embodiments, the diagnosis is based upon the presence and/or amount of one or more glycopeptides comprising the amino acid sequence of SEQ ID NOs: 229, 231-234, 236, 239, 241, 244-245, 247-255. In some embodiments, the diagnosis is based upon the presence and/or amount of one or more peptides and/or glycopeptide comprising the amino acid sequence of SEQ ID NOs: 229, 231-234, 236, 239, 241, 244-245, 247-255 set forth in Table 40. In some embodiments, the diagnosis is based upon the presence and/or amount of one or more glycopeptides comprising the amino acid sequence of SEQ ID NOs: 229, 231-234, 236, 239, 241, 244-245, 247-255 along with the associated glycan set forth in Table 35. In some embodiments, the diagnosis is based upon the presence and/or amount of two or more peptides and/or glycopeptides comprising the amino acid sequence of SEQ ID NOs: 229, 231-234, 236, 239, 241, 244-245, 247-255 along with the associated glycan set forth in Table 35. In some embodiments, the diagnosis is based upon the presence and/or amount of three or more peptides and/or glycopeptides comprising the amino acid sequence of SEQ ID NOs: 229, 231-234, 236, 239, 241, 244-245, 247-255 along with the associated glycan set forth in Table 35. In some embodiments, the diagnosis is based upon the presence and/or amount of four or more peptides and/or glycopeptides comprising the amino acid sequence of SEQ ID NOs: 229, 231-234, 236, 239, 241, 244-245, 247-255 along with the associated glycan set forth in Table 35. In some embodiments, the diagnosis is based upon the presence and/or amount of five or more peptides and/or glycopeptides comprising the amino acid sequence of SEQ ID NOs: 229, 231-234, 236, 239, 241, 244-245, 247-255 along with the associated glycan set forth in Table 35. In some embodiments, the diagnosis is based upon the presence and/or amount of 10 or more peptides and/or glycopeptides comprising the amino acid sequence of SEQ ID NOs: 229, 231-234, 236, 239, 241, 244-245, 247-255 along with the associated glycan set forth in Table 35. In some embodiments, the diagnosis is based upon the presence and/or amount of 15 or more peptides and/or glycopeptides comprising the amino acid sequence of SEQ ID NOs: 229, 231-234, 236, 239, 241, 244-245, 247-255 along with the associated glycan set forth in Table 35. In some embodiments, the diagnosis is based upon the presence and/or amount of each of the peptides and/or glycopeptides comprising the amino acid sequence of SEQ ID NOs: 229, 231-234, 236, 239, 241, 244-245, 247-255 along with the associated glycan set forth in Table 35. In some embodiments, the method comprises classifying a biological sample with respect to a plurality of states associated with NSCLC based upon one or more peptide and/or glycopeptide structure provided in Table 40 and selecting a treatment for NSCLC based upon the classification. In some embodiments, the method comprises classifying a biological sample with respect to a plurality of states associated with NSCLC based upon one or more peptide and/or glycopeptide structure provided in Table 40 and administering a treatment for NSCLC based upon the classification.

In some embodiments, the diagnosis is based upon the presence and/or amount of one or more peptides and/or glycopeptides consisting of the amino acid sequence of SEQ ID NOs: 229, 231-234, 236, 239, 241, 244-245, 247-255 set forth in Table 40. In some embodiments, the diagnosis is based upon the presence and/or amount of one or more glycopeptides consisting of the amino acid sequence of SEQ ID NOs: 229, 231-234, 236, 239, 241, 244-245, 247-255 set forth in Table 40. In some embodiments, the diagnosis is based upon the presence and/or amount of one or more glycopeptides consisting of the amino acid sequence of SEQ ID NOs: 229, 231-234, 236, 239, 241, 244-245, 247-255 along with the associated glycan set forth in Table 35. In some embodiments, the diagnosis is based upon the presence and/or amount of two or more peptides and/or glycopeptides consisting of the amino acid sequence of SEQ ID NOs: 229, 231-234, 236, 239, 241, 244-245, 247-255 along with the associated glycan set forth in Table 35. In some embodiments, the diagnosis is based upon the presence and/or amount of three or more peptides and/or glycopeptides consisting of the amino acid sequence of SEQ ID NOs: 229, 231-234, 236, 239, 241, 244-245, 247-255 along with the associated glycan set forth in Table 35. In some embodiments, the diagnosis is based upon the presence and/or amount of four or more peptides and/or glycopeptides consisting of the amino acid sequence of SEQ ID NOs: 229, 231-234, 236, 239, 241, 244-245, 247-255 along with the associated glycan set forth in Table 35. In some embodiments, the diagnosis is based upon the presence and/or amount of five or more peptides and/or glycopeptides consisting of the amino acid sequence of SEQ ID NOs: 229, 231-234, 236, 239, 241, 244-245, 247-255 along with the associated glycan set forth in Table 35. In some embodiments, the diagnosis is based upon the presence and/or amount of 10 or more peptides and/or glycopeptides consisting of the amino acid sequence of SEQ ID NOs: 229, 231-234, 236, 239, 241, 244-245, 247-255 along with the associated glycan set forth in Table 35. In some embodiments, the diagnosis is based upon the presence and/or amount of 15 or more peptides and/or glycopeptides consisting of the amino acid sequence of SEQ ID NOs: 229, 231-234, 236, 239, 241, 244-245, 247-255 along with the associated glycan set forth in Table 35. In some embodiments, the diagnosis is based upon the presence and/or amount of each of the peptides and/or glycopeptides consisting of the amino acid sequence of SEQ ID NOs: 229, 231-234, 236, 239, 241, 244-245, 247-255 along with the associated glycan set forth in Table 35. In some embodiments, the method comprises classifying a biological sample with respect to a plurality of states associated with NSCLC based upon one or more peptide and/or glycopeptide structure provided in Table 35 and selecting a treatment for NSCLC based upon the classification. In some embodiments, the method comprises classifying a biological sample with respect to a plurality of states associated with NSCLC based upon one or more peptide and/or glycopeptide structure provided in Table 35 and administering a treatment for NSCLC based upon the classification.

In some embodiments, the method comprises determining whether an individual has NSCLC by inputting quantification data from peptide and/or glycopeptide structure data for a set of peptide structures comprising one or more, two or more, three or more, four or more, five or more, 10 or more, 15 or more, or each of the peptides and/or glycopeptides consisting of the amino acid sequence of SEQ ID NOs: 229, 231-234, 236, 239, 241, 244-245, 247-255 into a machine-learning model trained to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the machine-learning model is a regularized regression model. In some embodiments, the regularized regression model comprises a LASSO regression model. In some embodiments, the peptide structures are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein.

In some embodiments, the method comprises determining whether an individual has NSCLC by inputting quantification data from peptide and/or glycopeptide structure data for a set of peptide structures comprising one or more, two or more, three or more, four or more, five or more, 10 or more, 15 or more, or each of the peptides and/or glycopeptides consisting of the amino acid sequence of SEQ ID NOs: 224-296 into a machine-learning model trained to identify a disease indicator, identifying by the machine-learning model, the disease indicator, and classifying the biological sample with respect to a plurality of states associated with NSCLC based upon the disease indicator. In some embodiments, the peptide structures are detected using LC-MS. In some embodiments, the LC-MS comprises LC-MS/MS and LC-MS/MS running in a MRM mode. In some embodiments, the quantification data from peptide and/or glycopeptide comprises a fold-change in peptide and/or glycopeptide abundance for different sample conditions (e.g., NSCLC vs healthy control sample). In some embodiments, the fold-change in peptide and/or glycopeptide abundance is greater than 1, wherein the peptide and/or glycopeptide abundance is increased in the NSCLC sample compared to healthy control sample. In some embodiments, the fold-change in peptide and/or glycopeptide abundance is less than 1, wherein the peptide and/or glycopeptide abundance is decreased in the NSCLC sample compared to healthy control sample. In some embodiments, the fold-change in peptide and/or glycopeptide abundance is equal to 1, wherein the peptide and/or glycopeptide abundance is the sample in the NSCLC sample and the healthy control sample. In some embodiments, the fold-change in peptide and/or glycopeptide abundance is about 1.0, about 1.2, about 1.4, about 1.6, about 1.8, about 2.0, about 2.2, about 2.4, about 2.6, about 2.8, about 3.0, about 3.5, about 4, about 5, about 6, about 7, about 8, about 9, or about 10 for the NSCLC sample compared to the healthy control sample. In some embodiments, the fold-change in peptide and/or glycopeptide abundance is more than 1.0, more than 1.2, more than 1.4, more than 1.6, more than 1.8, more than 2.0, more than 2.2, more than 2.4, more than 2.6, more than 2.8, more than 3.0, more than 3.5, more than 4, more than 5, more than 6, more than 7, more than 8, more than 9, or more than 10 for the NSCLC sample compared to the healthy control sample. In some embodiments, the fold-change in peptide and/or glycopeptide abundance is about 0.9, about 0.8, about 0.7, about 0.6, about 0.5, about 0.4, about 0.3, about 0.2, or about 0.1 for the NSCLC sample compared to the healthy control sample. In some embodiments, the fold-change in peptide and/or glycopeptide abundance is less than 0.9, less than 0.8, less than 0.7, less than 0.6, less than 0.5, less than 0.4, less than 0.3, less than 0.2, or less than 0.1 for the NSCLC sample compared to the healthy control sample. In some embodiments, the fold-change in peptide and/or glycopeptide abundance is provided for the amino acid sequence of SEQ ID NOs: 224-296 as set forth by Table 38A and Table 38B. In some embodiments, the method comprises selecting a treatment for NSCLC based upon the quantification data described herein. In some embodiments, the method comprises administering a treatment for NSCLC based upon the quantification data described herein. In some embodiments, the diagnostic methods herein comprise selecting one or more glycopeptide based upon a fold change cutoff between healthy control samples and NSCLC samples. In some embodiments, the selected glycopeptides are used to train a model to diagnose NSCLC.

In some embodiments, the method comprises determining whether an individual has NSCLC by inputting quantification data from peptide and/or glycopeptide structure data for a set of peptide structures comprising one or more, two or more, three or more, four or more, five or more, 10 or more, 15 or more, or each of the peptides and/or glycopeptides consisting of the amino acid sequence of SEQ ID NOs: 224-257 into a machine-learning model, wherein the machine-learning model comprises a regularized regression model (e.g., LASSO regression model) trained using one or more of a first set of peptide and/or glycopeptide structure coefficients set forth in Table 39 for all stages of NSCLC (stages 1-4), a second set of peptide and/or glycopeptide structure coefficients set forth in Table 39 for early-stage NSCLC (stages 1-2), and a third set of peptide structure coefficients set forth in Table 39 for late-stage NSCLC (stages 3-4). In some embodiments, the first set of peptide structure coefficients used in training the model comprises the amino acid sequence of SEQ ID NOs: 229, 231-234, 236, 239, 241, 244-245, 247-255. In some embodiments, the second set of peptide structure coefficients used in training the model comprises the amino acid sequence of SEQ ID NOs: 224, 227, 233, 238, 240-242, 244, 247-248, 250-255. In some embodiments, the third set of peptide structure coefficients used in training the model comprise the amino acid sequence of SEQ ID NOs: 225-226, 228-230, 233-237, 239, 241-244, 246, 254-257. In some embodiments, the machine-learning model is trained using any one of the set of peptide structure coefficients described herein to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the machine-learning model trained using the first set of peptide structure coefficients is a regularized regression model (e.g., LASSO regression model). In some embodiments, the machine-learning model trained using the second set of peptide structure coefficients is a regularized regression model (e.g., LASSO regression model). In some embodiments, the machine-learning model trained using the third set of peptide structure coefficients is a regularized regression model (e.g., LASSO regression model). In some embodiments, the machine-learning model trained using the first set of peptide structure coefficients comprises a regularized regression model (e.g., LASSO regression model). In some embodiments, the machine-learning model trained using the second set of peptide structure coefficients comprises a regularized regression (e.g., LASSO regression model). In some embodiments, the machine-learning model trained using the third set of peptide structure coefficients comprises a regularized regression model (e.g., LASSO regression model). In some embodiments, the peptide structures are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein.

In some embodiments, the method comprises determining whether an individual has NSCLC by inputting quantification data from peptide structure data for a set of peptide structures comprising one or more, two or more, three or more, four or more, five or more, 10 or more, 15 or more, or each of the peptides and/or glycopeptides consisting of the amino acid sequence of SEQ ID NOs: 224-257 into a machine-learning model, wherein the machine-learning model consists of a regularized regression model (e.g., LASSO regression model) trained using a first set of peptide structure coefficients set forth in Table 39 for all stages of NSCLC (stages 1-4). In some embodiments, the first set of peptide structure coefficients used in training the model consist of the amino acid sequence of SEQ ID NOs: 229, 231-234, 236, 239, 241, 244-245, 247-255. In some embodiments, the model is trained using the first set of peptide structure coefficients to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the machine-learning model trained using the first set of peptide structure coefficients is a regularized regression model. In some embodiments, the regularized regression model trained using the first set of peptide structure coefficients consists of a LASSO regression model. In some embodiments, the peptide structures are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein.

In some embodiments, the method comprises determining whether an individual has NSCLC by inputting quantification data from peptide structure data for a set of peptide structures comprising one or more, two or more, three or more, four or more, five or more, 10 or more, 15 or more, or each of the peptides and/or glycopeptides consisting of the amino acid sequence of SEQ ID NOs: 224-257 into a machine-learning model, wherein the machine-learning model consists of a regularized regression model (e.g., LASSO regression model) trained using a second set of peptide structure coefficients set forth in Table 39 for early-stage NSCLC (stages 1-2). In some embodiments, the second set of peptide structure coefficients used in training the machine-learning model consist of the amino acid sequence of SEQ ID NOs: 224, 227, 233, 238, 240-242, 244, 247-248, 250-255. In some embodiments, the machine-learning model is trained using the second set of peptide structure coefficients to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the machine-learning model trained using the second set of peptide structure coefficients is a regularized regression model. In some embodiments, the regularized regression model trained using the second set of peptide structure coefficients consists of a LASSO regression model. In some embodiments, the peptide structure are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein.

In some embodiments, the method comprises determining whether an individual has NSCLC by inputting quantification data from peptide structure data for a set of peptide structures comprising one or more, two or more, three or more, four or more, five or more, 10 or more, 15 or more, or each of the peptides and/or glycopeptides consisting of the amino acid sequence of SEQ ID NOs: 224-257 into a machine-learning model, wherein the machine-learning model consists of a regularized regression model (e.g., LASSO regression model) trained using a third set of peptide structure coefficients set forth in Table 39 for late-stage NSCLC (stages 3-4). In some embodiments, the third set of peptide structure coefficients used in training the model consist of the amino acid sequence of SEQ ID NOs: 225-226, 228-230, 233-237, 239, 241-244, 246, 254-257. In some embodiments, the third set of peptide structure coefficients used in training the model consist of the amino acid sequence of SEQ ID NOs: 225-226, 228-230, 233-237, 239, 241-244, 246, 254-257. In some embodiments, the model is trained using the third set of peptide structure coefficients to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the machine-learning model trained using the third set of peptide structure coefficients is a regularized regression model. In some embodiments, the regularized regression model trained using the third set of peptide structure coefficients consists of a LASSO regression model. In some embodiments, the peptide structure are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein.

In some embodiments, the method further comprises collecting a biological sample. In some embodiments, the biological sample comprises a blood sample, a serum sample, a buccal epithelia sample, a nasal epithelia sample, a sputum cytology sample, and a bronchial biopsy sample. In some embodiments, the biological sample comprises a pre-treatment peripheral blood sample. In some embodiments, the biological sample is collected from an individual with early-stage NSCLC. In some embodiments, the biological sample is collected from an individual with late-stage NSCLC. In some embodiments, the biological sample is collected from an individual with stage 0, stage I, stage II, stage III, or stage IV NSCLC. In some embodiments, the biological sample is from a healthy individual (i.e., an individual who does not have NSCLC). In some embodiments, the biological sample is collected from an individual before the onset of disease symptoms and pathological conditions of NSCLC. In some embodiments, the biological sample is collected from an individual after the onset of disease symptoms and pathological conditions of NSCLC. In some embodiments, the biological sample is collected from an individual during disease symptoms and pathological conditions of NSCLC. In some embodiments, the biological sample is collected from an individual before disease progression of NSCLC.

For example, in certain embodiments, the presence or amount of the at least one peptide and/or glycopeptide structure is detected using liquid chromatography-tandem mass spectrometry (LC-MS/MS) running in a multiple reaction monitoring (MRM) mode, or an ELISA. In one embodiment, the at least one peptide and/or glycopeptide structure is none, or below a detection limit. In one embodiment, the at least one peptide and/or glycopeptide structure that is below a detection limit is enriched and/or concentrated in a biological sample using an antibody specific to the at least one peptide and/or glycopeptide structure. In one embodiment, the at least one peptide and/or glycopeptide structure that is below a detection limit is enriched and/or concentrated in a biological sample using a protein such as a lectin that binds to a carbohydrate portion of at least one glycopeptide structure. In various embodiments, the antibody or protein configured to bind carbohydrate portions (e.g., lectin) is immobilized onto a bead substrate that is optionally a magnetic bead substrate. In one embodiment, the NSCLC is late-stage NSCLC. In one embodiment, the biological sample is a pre-treatment peripheral blood sample. In one embodiment, the one or more peptide structure includes a glycopeptide of a lung-specific protein, and the at least one peptide structure comprises three or more peptide structure identified in Table 35. In one embodiment, the at least one peptide structure comprises three or more amino acid sequence of SEQ ID NOs: 224-257 set forth in Table 35. In one embodiment, the at least one peptide structure comprises three or more amino acid sequence of SEQ ID NOs: 224-257 along with the associated glycan set forth in Table 35. In one embodiment, the at least one peptide structure comprises three or more amino acid sequence of SEQ ID NOs: 258-296 set forth in Table 35. In one embodiment, the at least one peptide structure comprises three or more amino acid sequence of SEQ ID NOs: 258-296 along with the associated glycan set forth in Table 35. In one embodiment, the at least one peptide structure comprises three or more peptide structures identified in Table 40. In one embodiment, the at least one peptide structure comprises three or more amino acid sequence of SEQ ID NOs: 229, 231-234, 236, 239, 241, 244-245, 247-255 set forth in Table 40. In one embodiment, the at least one peptide structure comprises three or more amino acid sequence of SEQ ID NOs: 229, 231-234, 236, 239, 241, 244-245, 247-255 along with the associated glycan set forth in Table 35.

In some embodiments, the method comprises assessing one or more clinical indicators of NSCLC. In some embodiments, the one or more clinical indicator of NSCLC comprises a new cough, a worsening cough, a persistent cough, a cough that produces blood (e.g., hemoptysis), shortness of breath, a persistent chest infection (e.g., bronchitis, pneumonia), wheezing, chest pain, voice hoarseness, headache, facial swelling, body swelling, upper body pain, weakness of the hand, a droopy eyelid, and blurred vision. In some embodiments, the method includes assessing a patient for unexplained weight loss, tiredness, fatigue, persistent or worsening bone pain, and persistent or worsening joint pain. In some embodiments, the method includes assessing the clinical indicators described herein and any combination thereof in diagnosing a patient having NSCLC. In some embodiments, the method comprises classifying a biological sample with respect to a plurality of states associated with NSCLC based upon one or more peptides structure provided in Table 35 and/or Table 40. In some embodiments, the method comprises classifying a biological sample with respect to a plurality of states associated with NSCLC based upon one or more glycopeptides provided in Table 35 and/or Table 40. In some embodiments, the diagnosis is based upon the presence and/or amount of each of the peptides and/or glycopeptides comprising the amino acid sequence of SEQ ID NOs: 224-296. In some embodiments, the diagnosis is based upon the presence and/or amount of each of the glycopeptides comprising the amino acid sequence of SEQ ID NOs: 224-296. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptide is determined by MRM-MS. In some embodiments, the method comprises classifying a biological sample with respect to a plurality of states associated with NSCLC, based upon one or more peptide structure provided in Table 35 or Table 40, and selecting a treatment for NSCLC based upon the classification. In some embodiments, the method comprises classifying a biological sample with respect to a plurality of states associated with NSCLC, based upon one or more peptide structure provided in Table 35 or Table 40, and administering a treatment for NSCLC based upon the classification.

In some embodiments, the method comprises assessing one or more risk factors selected from the group comprising tobacco smoking, environmental and occupational carcinogen exposure (e.g., tar, soot, arsenic, chromium, and nickel, asbestos, radiation, radon), a family history of lung cancer, poor diet, and limited physical activity. In certain embodiments, the method comprises assessing one or more risk factors of NSCLC, wherein the risk factor of NSCLC is selected from the group consisting of tobacco smoking, a family history of lung cancer, environmental and occupational carcinogen exposure, poor diet, and limited physical activity. In certain embodiments, the individual is determined have a healthy state, in which a healthy state may include the absence of NSCLC. In some embodiments, the method comprises classifying a biological sample with respect to a plurality of states associated with NSCLC based upon one or more peptide structures provided in Table 35 or Table 40. In some embodiments, the diagnosis is based upon the presence and/or amount of each of the peptides and/or glycopeptides comprising the amino acid sequence of SEQ ID NOs: 224-296. In some embodiments, the diagnosis is based upon the presence and/or amount of each of the glycopeptides comprising the amino acid sequence of SEQ ID NOs: 224-296 In some embodiments, the diagnosis is based upon the presence and/or amount of each of the peptides and/or glycopeptides comprising the amino acid sequence of SEQ ID NOs: 224-296 along with the associated glycan set forth in Table 35. In some embodiments, the diagnosis is based upon the presence and/or amount of each of the peptides and/or glycopeptides comprising the amino acid sequence of SEQ ID NOs: 229, 231-234, 236, 239, 241, 244-245, 247-255. In some embodiments, the diagnosis is based upon the presence and/or amount of each of the glycopeptides comprising the amino acid sequence of SEQ ID NOs: 229, 231-234, 236, 239, 241, 244-245, 247-255. In some embodiments, the diagnosis is based upon the presence and/or amount of each of the peptides and/or glycopeptides comprising the amino acid sequence of SEQ ID NOs: 229, 231-234, 236, 239, 241, 244-245, 247-255 along with the associated glycan set forth in Table 35. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptide is determined by MRM-MS. In some embodiments, the method comprises classifying a biological sample with respect to a plurality of states associated with NSCLC, based upon one or more peptide structure provided in Table 35 or Table 40, and selecting a treatment for NSCLC based upon the classification. In some embodiments, the method comprises classifying a biological sample with respect to a plurality of states associated with NSCLC, based upon one or more peptide structure provided in Table 35 or Table 40, and administering a treatment for NSCLC based upon the classification.

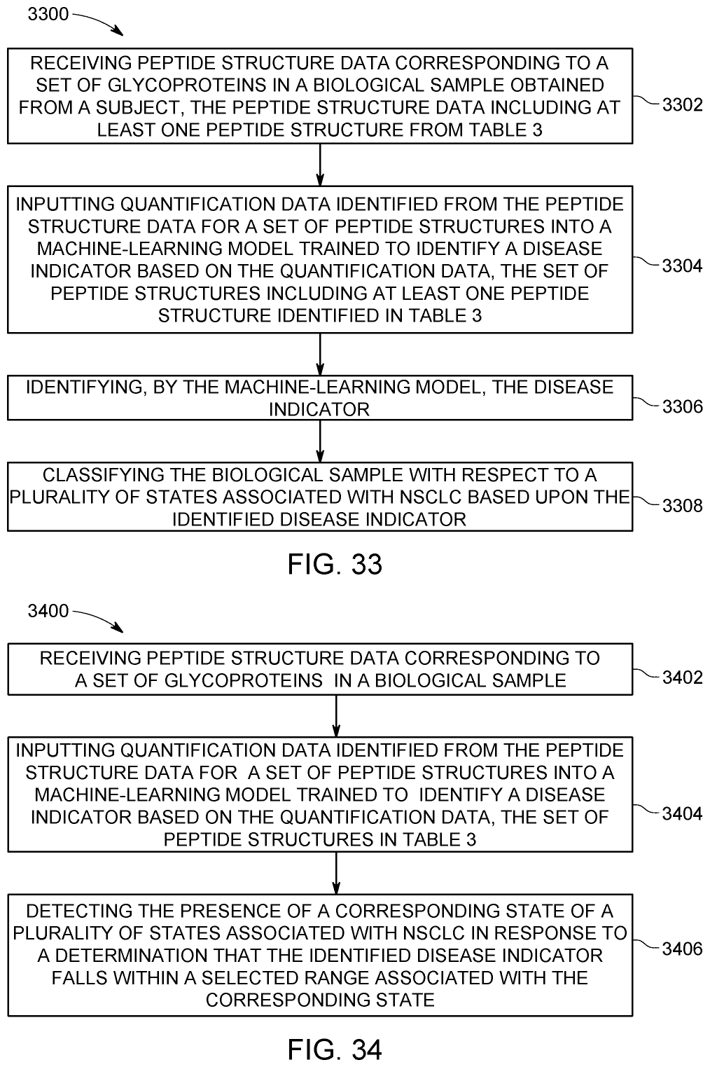

FIG. 33 illustrates a flow diagram 3300 of a method for classifying a biological sample obtained from a subject with respect to a plurality of states associated with NSCLC, in accordance with the presently disclosed embodiments. The flow diagram 3300 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 3300 may begin at block 3302 with one or more processing devices (e.g., computing platform 302) receiving peptide structure data corresponding to a set of glycoproteins in the biological sample. The flow diagram 3300 may then continue at block 3304 with one or more processing devices (e.g., computing platform 302) inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 35 and/or Table 40. The flow diagram 3300 may then continue at block 3306 with one or more processing devices (e.g., computing platform 302) identifying, by the machine-learning model, the disease indicator. The flow diagram 3300 may then conclude at block 3308 with one or more processing devices (e.g., computing platform 302) classifying the biological sample with respect to a plurality of states associated with NSCLC based upon the identified disease indicator.

FIG. 34 illustrates a flow diagram 3400 of a method for detecting the presence of one of a plurality of states associated with NSCLC in a subject, in accordance with the presently disclosed embodiments. The flow diagram 3400 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 3400 may begin at block 3402 with one or more processing devices (e.g., computing platform 302) receiving peptide structure data corresponding to a set of glycoproteins in a biological sample obtained from a subject, wherein the peptide structure data comprises at least one peptide structure from Table 35 and/or Table 40. The flow diagram 3400 may then continue at block 3404 with one or more processing devices (e.g., computing platform 302) inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 35 and/or Table 40. The flow diagram 3400 may then conclude at block 3406 with one or more processing devices (e.g., computing platform 302) detecting the presence of a corresponding state of the plurality of states associated with NSCLC in response to a determination that the identified disease indicator falls within a selected range associated with the corresponding state.

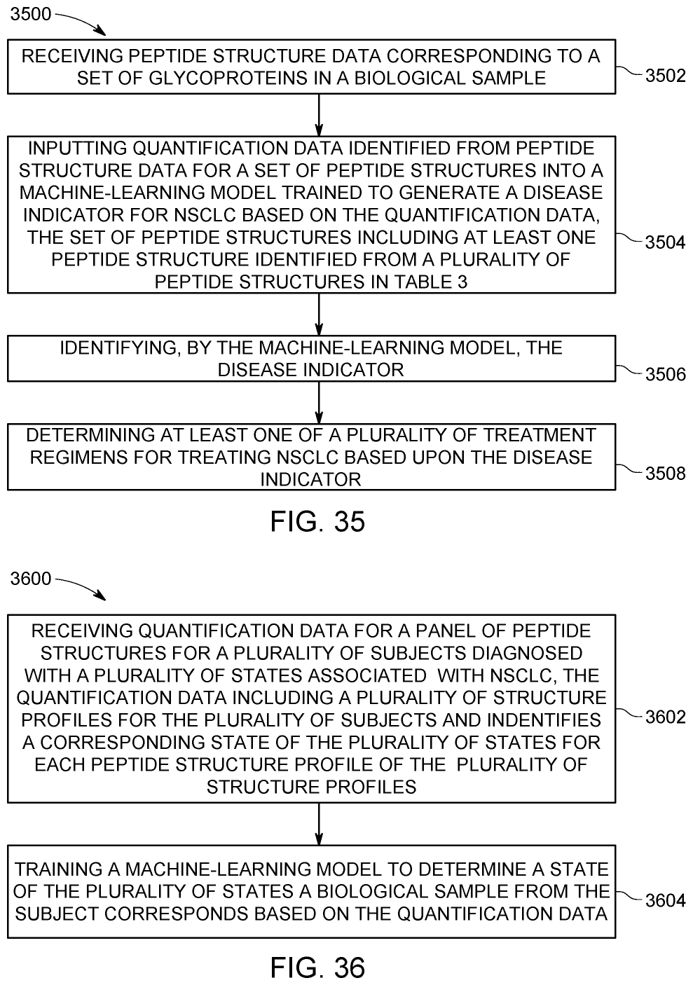

FIG. 35 illustrates a flow diagram 3500 of a method determining one or more of a plurality of treatment regimens for treating NSCLC in a subject, in accordance with the presently disclosed embodiments. The flow diagram 3500 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 3500 may begin at block 3502 with one or more processing devices (e.g., computing platform 302) receiving peptide structure data corresponding to a set of glycoproteins in the biological sample. The flow diagram 3500 may then continue at block 3504 with one or more processing devices (e.g., computing platform 302) inputting quantification data identified from the peptide structure data for a set of the peptide structures into a machine-learning model trained to identify a disease indicator for NSCLC based on the quantification data, wherein the set of peptide structure data comprises at least one peptide structure identified from a plurality of peptide structures in Table 35 and/or Table 40. The flow diagram 3500 may then continue at block 3506 with one or more processing devices (e.g., computing platform 302) identifying, by the machine-learning model, the disease indicator. The flow diagram 3500 may then conclude at block 3508 with one or more processing devices (e.g., computing platform 302) determining at least one of a plurality of treatment regimens for treating NSCLC based upon the identified disease indicator.

### VI.C. General Methodology System for Diagnosis of Ovarian Cancer based on Tables 41 and 42

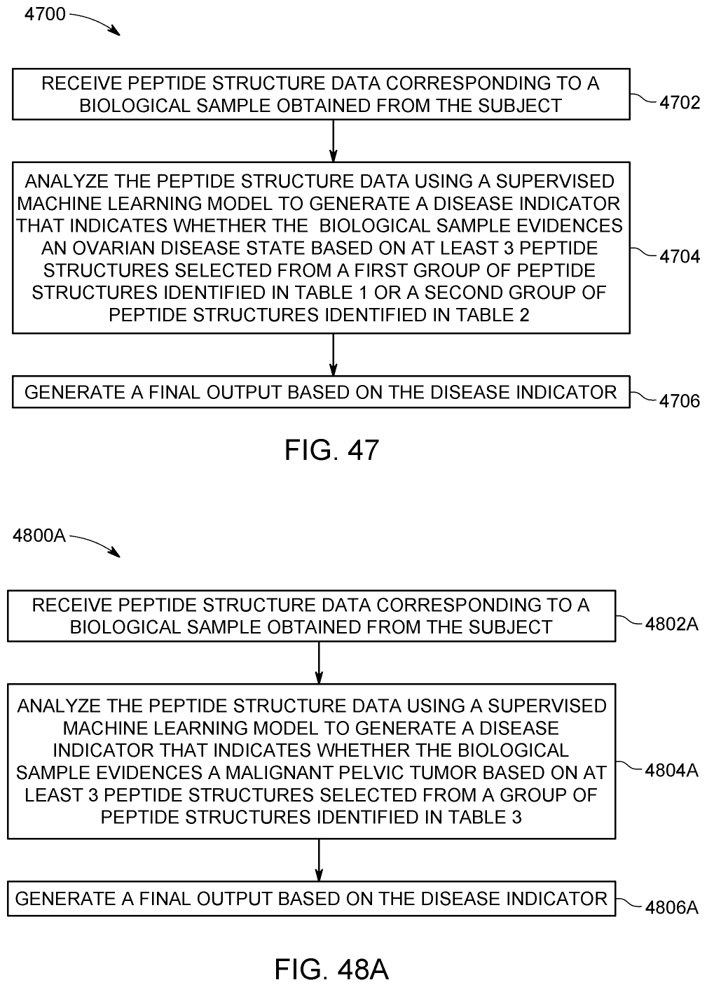

FIG. 47 is a flowchart of a process for diagnosing a subject with respect to an ovarian cancer disease state in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate a final output that includes at least a diagnosis output for the subject.

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 41 or Table 42, with the peptide sequence being one of SEQ ID NOS: 307-316 in Table 41 or one of SEQ ID NOS: 310, 311, and 428-443 in Table 42, the SEQ ID NOS being defined in Table 45 below.

Step 504 includes analyzing the peptide structure data using a supervised machine learning model to generate a disease indicator that indicates whether the biological sample evidences an ovarian cancer disease state based on at least three peptide structures selected from a first group of peptide structures identified in Table 41 (below) or a second group of peptide structures identified in Table 42 (below). In step 504, the first and second groups of peptide structures are associated with the ovarian cancer disease state. The first group of peptide structures is listed in Table 41 with respect to relative significance to the disease indicator. The second group of peptide structures is listed in Table 42 with respect to relative significance to the disease indicator.

The first group of peptide structures in Table 41 includes peptide structures that have been determined relevant to distinguishing at least between ovarian cancer (e.g., EOC) and a healthy state. For example, the first group of peptide structures may be used to predict the probability of EOC for use in clinically screening patients. In one or more embodiments, the first group of peptide structures in Table 41 may also be peptide structures that have been determined relevant to distinguishing between ovarian cancer (e.g., EOC) and a benign tumor state (e.g., a benign pelvic tumor). For example, the first group of peptide structures may be used to clinically triage patients that have been identified as having pelvic tumors to determine the probability that such a tumor evidences EOC.

The second group of peptide structures in Table 42 includes peptide structures that have been determined relevant to distinguishing at least between ovarian cancer (e.g., EOC) and the benign tumor state (e.g., a benign pelvic tumor). For example, the second group of peptide structures may be used to clinically triage patients that have been identified as having pelvic tumors to determine the probability that such a tumor evidences EOC. In this manner, the second group of peptide structures may predict malignancy of an identified pelvic tumor.

In one or more embodiments, the at least 3 peptide structures includes at least 3, at least 4, at least 5, at least 6, at least 7, at least 8, at least 9, or all 10 of the peptide structures PS-165 to PS-174 in Table 41. In some embodiments, the at least 3 peptide structures include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures PS-169 and PS-175 through PS-198 in Table 41. In some embodiments, the at least 3 peptide structures includes at least PS-169, which is present in both Table 41 and Table 42.

In one or more embodiments, step 504 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 504 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature-relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for the ovarian cancer disease state and the supervised machine learning model is configured to generate an output that identifies the biological sample as either evidencing (“positive for”) the ovarian cancer disease state when the disease indicator is greater than a selected threshold or not evidencing (“negative for”) the ovarian cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 506 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the ovarian cancer disease state if the biological sample evidences the ovarian cancer disease state based on the disease indicator. The diagnosis may be, for example, “negative” if the biological sample does not evidence the ovarian cancer disease state based on the disease indicator. A negative diagnosis may mean that the biological sample has a non-ovarian cancer state. The negative diagnosis for the ovarian cancer disease state can include at least one of a healthy state, a benign tumor state, or some other non-malignant state.

Generating the diagnosis output in step 506 may include determining that the score falls above (or at or above) a selected threshold and generating a positive diagnosis for the ovarian cancer disease state. Alternatively, step 506 can include determining that the score falls below (or at or below) a selected threshold and generating a negative diagnosis for the ovarian cancer disease state. In some scoring systems, the score can include a probability score and the selected threshold can be 0.5. In other scoring systems, the selected threshold can fall within a range between 0.30 and 0.65.

In one or more embodiments, the final output in step 506 may include a treatment output if the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table 41 below lists a first group of peptide structures associated with malignant pelvic tumors (e.g., ovarian cancer such as EOC). One or more features (e.g., relative abundance, concentration, site occupancy) of these peptide structures may be used in the supervised machine learning model described above to generate a disease indicator that predicts the probability of malignancy (e.g., in the context of screening for malignant pelvic tumors). The first group of peptide structures is listed in Table 41 in order with respect to relative significance to the disease indicator. In training, testing, and predictive use of this model, the quantification metrics for peptide structure PS-173, peptide structure PS-174, or a combination of the two may form one input. Table 41 also identifies check markers CK-1 and CK-2, which may also be used by the model.

TABLE 41
1^{st} Group of Peptide Structures Associated with
Ovarian Cancer
(may be used to distinguish between malignant
pelvic tumor (e.g., EOC) and healthy)

| | | | | | Linking | Linking | Glycan |
|---|---|---|---|---|---|---|---|
| | Peptide | (Protein) | (Peptide) | Mono- | Site | Site | Struc- |
| PS- | Structure | SEQ | SEQ | isotopic | Pos. in | Pos. in | ture |
| ID | (PS) | ID | ID | mass | Protein | Peptide | GL |
| NO. | NAME | NO. | NO. | (Da) | Sequence | Sequence | NO. |
| 165 | ZA2G_ | 297 | 307 | 3342.26 | 128 | 8 | 5402 |
| | 128_ | | | | | | |
| | 5402 | | | | | | |
| 166 | IC1_ | 298 | 308 | 4961.09 | 253 | 4 | 6503 |
| | 253_ | | | | | | |
| | 6503 | | | | | | |
| 167 | CFAI_ | 299 | 309 | 3025.18 | 494 | 4 | 5402 |
| | 494_ | | | | | | |
| | 5402 | | | | | | |
| 168 | CERU_ | 300 | 310 | 4898.89 | 138 | 10 | 6513 |
| | 138_ | | | | | | |
| | 6513 | | | | | | |
| 169 | IGG1_ | 301 | 311 | 2633.04 | 180 | 5 | 3410 |
| | 297_ | | | | | | |
| | 3410 | | | | | | |
| 170 | HEMO_ | 302 | 312 | 4731.84 | 64 | 15 | 5402 |
| | 64_ | | | | | | |
| | 5402 | | | | | | |
| 171 | APOB_ | 303 | 313 | 5754.34 | 983 | 16 | 5402 |
| | 983_ | | | | | | |
| | 5402 | | | | | | |
| 172 | HPT_ | 304 | 314 | 6888.63 | 207 | 5, | 121005 |
| | 207_ | | | | | 9 | |
| | 121005 | | | | | | |
| CK- | FINC_ | N/A | N/A | N/A | N/A | N/A | N/A |
| 1 | SYTITG | | | | | | |
| | LQPGTD | | | | | | |
| | YK | | | | | | |
| 173 | IGG3_ | 305 | 315 | 2470.99 | 227 | 5 | 3400 |
| | 297_ | | | | | | |
| | 3400 | | | | | | |
| 174 | IGG34_ | 306 | 316 | 2470.99 | 227 | 5 | 3400 |
| | 297_ | | | | | | |
| | 3400 | | | | | | |
| CK- | APOM_ | N/A | N/A | N/A | N/A | N/A | N/A |
| 2 | 135_ | | | | | | |
| | 8500 | | | | | | |
| | CHK | | | | | | |

Table 42 below lists a second group of peptide structures associated with malignant pelvic tumors (e.g., ovarian cancer such as EOC). One or more features (e.g., relative abundance, concentration, site occupancy) of these peptide structures may be used in the supervised machine learning model described above to generate a disease indicator that predicts the probability of malignancy (e.g., in the context of triaging to distinguish between malignant and benign pelvic tumors). The second group of peptide structures is listed in Table 42 in order with respect to relative significance to the disease indicator. Table 42 also identifies check markers CK-3 and CK-4, which may also be used by the model.

TABLE 42
2^{nd} Group of Peptide Structures Associated with Ovarian Cancer
(may be used to distinguish between malignant v. benign pelvic tumors)

| | Peptide | | | Mono- | Linking | Linking | |
|---|---|---|---|---|---|---|---|
| | Structure | (Protein) | (Peptide) | isotopic | Site Pos. | Site Pos. | Glycan |
| PS-ID | (PS) | SEQ ID | SEQ ID | mass | in Protein | in Peptide | Structure |
| NO. | NAME | NO. | NO. | (Da) | Sequence | Sequence | GL NO. |
| CK-3 | APOD_98_9800_CHECK | N/A | N/A | N/A | N/A | N/A | N/A |
| 175 | CO2_621_5200 | 417 | 428 | 2670.19 | 621 | 11 | 5200 |
| 169 | IGG1_297_3410 | 301 | 311 | 2633.04 | 180 | 5 | 3410 |
| 176 | AGP1_93_7612 | 418 | 429 | 4995.98 | 93 | 7 | 7612 |
| 177 | AACT_271_7602 | 419 | 430 | 4686.91 | 271 | 4 | 7602 |
| 178 | A2MG_1424_5402 | 420 | 431 | 4366.95 | 1424 | 3 | 5402 |
| 179 | AACT_271_6513 | 419 | 430 | 4758.93 | 271 | 4 | 6513 |
| 180 | CERU_397_5402 | 300 | 432 | 4330.76 | 397 | 2 | 5402 |
| 181 | APOB_3411_5301 | 303 | 433 | 3316.40 | 3411 | 7 | 5301 |
| 182 | AACT_106_6513 | 419 | 434 | 5406.24 | 106 | 2 | 6513 |
| 183 | CERU_138_5402 | 300 | 310 | 4096.61 | 138 | 10 | 5402 |
| 184 | A1AT_107_6513 | 421 | 435 | 6697.87 | 107 | 14 | 6513 |
| 185 | AGP1_93_7602 | 418 | 429 | 4849.93 | 93 | 7 | 7602 |
| 186 | VTNC_242_6502 | 422 | 436 | 5341.22 | 242 | 1 | 6502 |
| 187 | IGG2_297_3510 | 423 | 437 | 2804.13 | 176 | 5 | 3510 |
| 188 | CFAH_882_5411 | 424 | 438 | 4079.71 | 882 | 15 | 5411 |
| CK-4 | APOM_135_8500_CHECK | N/A | N/A | N/A | N/A | N/A | N/A |
| 189 | AGP1_103_8704 | 418 | 439 | 4657.74 | 103 | 2 | 8704 |
| 190 | IGG1_297_4300 | 301 | 311 | 2445.95 | 180 | 5 | 4300 |
| 191 | APOH_253_5401 | 425 | 440 | 3163.24 | 253 | 3 | 5401 |
| 192 | APOD_98_5411 | 426 | 441 | 4312.85 | 98 | 16 | 5411 |
| 193 | TRFE_630_5411 | 427 | 442 | 4573.85 | 630 | 9 | 5411 |
| 194 | CERU_138_6502 | 300 | 310 | 4461.74 | 138 | 10 | 6502 |
| 195 | A2MG_1424_5411 | 420 | 431 | 4221.91 | 1424 | 3 | 5411 |
| 196 | A2MG_55_5411 | 420 | 443 | 4455.96 | 55 | 9 | 5411 |
| 197 | TRFE_630_5412 | 427 | 442 | 4864.95 | 630 | 9 | 5412 |
| 198 | IGG2_297_4511 | 423 | 437 | 3257.28 | 176 | 5 | 4511 |

### VI.D. General Methodology System for Diagnosis of Ovarian Cancer based on Table 43A

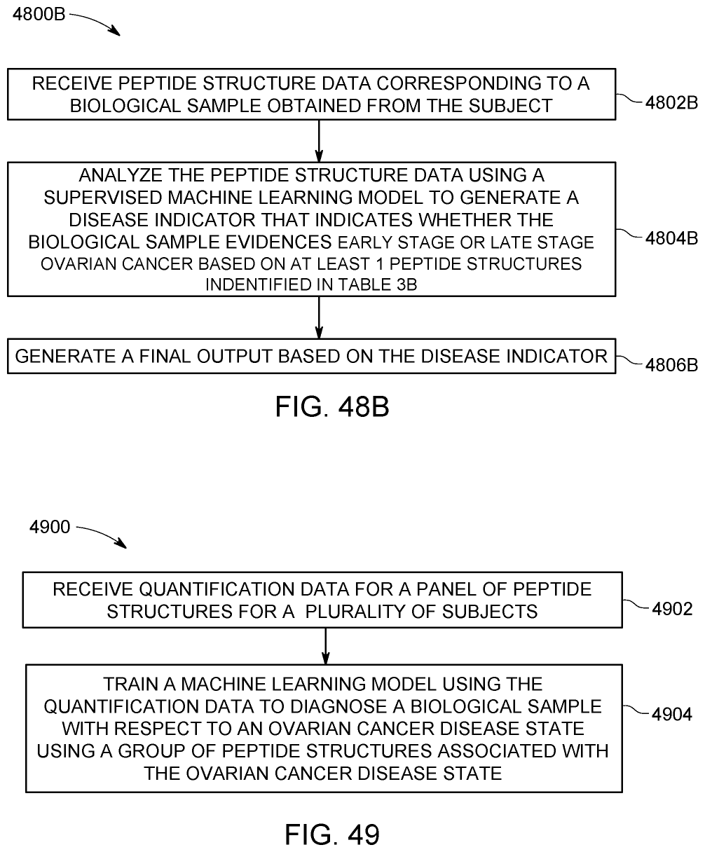

FIG. 48A is a flowchart of a process for diagnosing a subject with respect to an ovarian cancer disease state in accordance with one or more embodiments. Process 600 may be implemented using, for example, at least a portion of workflow 100 as described in FIGS. 1, 2A, and 2B and/or analysis system 300 as described in FIG. 3. Process 600 may be used to generate a final output that includes at least a diagnosis output for the subject.

Step 602 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 43A, with the peptide sequence being one of SEQ ID NOS: 307, 310, 311, 428-431, 434, 435, 437, 439, 441, 442, 443, 450-462 in Table 43A, the SEQ ID NOS being defined in Table 45 below.

Step 604 includes analyzing the peptide structure data using a supervised machine learning model to generate a disease indicator that predicts whether the biological sample evidences a malignant pelvic tumor or benign pelvic tumor based on at least three peptide structures selected from a group of peptide structures identified in Table 43A. The group of peptide structures is listed in Table 43A with respect to relative significance to the disease indicator, which may be a probability score. In step 604, the group of peptide structures is associated with the malignancy (e.g., EOC). For example, the group of peptide structures in Table 43A includes peptide structures that have been determined relevant to distinguishing between a malignant and benign nature of a pelvic tumor.

In one or more embodiments, the at least 3 peptide structures includes at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures PS-165, PS-169, PS-175, PS-179, PS-184, PS-189, PS-192, PS-193, PS-194, PS-195, PS-196, and PS-199 to PS-225 identified in Table 43A.

In one or more embodiments, step 604 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 604 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is evidences malignancy (e.g., EOC) and the supervised machine learning model is configured to generate an output that identifies the biological sample as either evidencing (“positive for”) malignancy when the disease indicator is greater than a selected threshold or not evidencing (“negative for”) malignancy when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 606 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for an ovarian cancer disease state (e.g., EOC) if the biological sample evidences malignancy based on the disease indicator. The diagnosis may be, for example, “negative” if the biological sample does not evidence malignancy based on the disease indicator. A negative diagnosis may mean that the biological sample evidences a benign status (or a non-ovarian cancer state).

Generating the diagnosis output in step 606 may include determining that the score falls above (or at or above) a selected threshold and generating a positive diagnosis for the ovarian cancer disease state. Alternatively, step 606 can include determining that the score falls below (or at or below) a selected threshold and generating a negative diagnosis for the ovarian cancer disease state. In some scoring systems, the score can include a probability score and the selected threshold can be 0.5. In other scoring systems, the selected threshold can fall within a range between 0.30 and 0.65.

In one or more embodiments, the final output in step 606 may include a treatment output if the disease indicator predicts malignancy and/or the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

TABLE 43A
3^{rd} Group of Peptide Structures Associated with Ovarian Cancer
(may be used to distinguish between malignant and benign pelvic tumors)

| | | | | Linking | |
|---|---|---|---|---|---|
| | | (Protein) | (Peptide) | Site Pos. | Glycan |
| PS-ID | | SEQ ID | SEQ ID | in Protein | Structure |
| NO. | Peptide Structure (PS) NAME | NO. | NO. | Sequence | GL NO. |
| 199 | VTNC_169_5401 | 422 | 450 | 169 | 5401 |
| 200 | FETUA_176_6513 | 444 | 451 | 176 | 6513 |
| 201 | AGP1_93_7614 | 418 | 429 | 93 | 7614 |
| 202 | QUANTPEP.A2GL_DLLLPQPDLR | 445 | 452 | N/A | N/A |
| 203 | HPT_184_5402 | 304 | 453 | 184 | 5402 |
| 204 | TRFE_432_6503 | 427 | 454 | 432 | 6503 |
| 205 | TRFE_630_6513 | 427 | 442 | 630 | 6513 |
| 206 | HEMO_453_5402 | 302 | 455 | 453 | 5402 |
| 207 | QUANTPEP.TTR_TSESGELHGLT | 446 | 456 | N/A | N/A |
| | TEEEFVEGIYK | | | | |
| 169 | IGG1_297_3410 | 301 | 311 | 297 | 3410 |
| 109 | TRFE_630_5400 | 427 | 442 | 630 | 5400 |
| 208 | AGP1_103_9804 | 418 | 439 | 103 | 9804 |
| 209 | TRFE_432_6501 | 427 | 454 | 432 | 6501 |
| 210 | HPT_241_5402 | 304 | 457 | 241 | 5402 |
| 211 | IGG1_297_5510 | 301 | 311 | 297 | 5510 |
| 212 | QUANTPEP.AFAM_SDVGFLPPFP | 447 | 458 | N/A | N/A |
| | TLDPEEK | | | | |
| 196 | A2MG_55_5411 | 420 | 443 | 55 | 5411 |
| 214 | IGG2_297_5510 | 423 | 437 | 297 | 5510 |
| 215 | AGP1_103_7603 | 418 | 439 | 103 | 7603 |
| 216 | IGG2_297_5400 | 423 | 437 | 297 | 5400 |
| 165 | ZA2G_128_5402 | 297 | 307 | 128 | 5402 |
| 217 | TRFE_630_6502 | 427 | 442 | 630 | 6502 |
| 218 | TRFE_432_6502 | 427 | 454 | 432 | 6502 |
| 219 | IGG2_297_4510 | 423 | 437 | 297 | 4510 |
| 220 | AACT_106_7614 | 419 | 434 | 106 | 7614 |
| 221 | PEP-APOA1_VSFLSALEEYTK | 448 | 459 | N/A | N/A |
| 175 | CO2_621_5200 | 417 | 428 | 621 | 5200 |
| 179 | AACT_271_6513 | 419 | 430 | 271 | 6513 |
| 222 | FETUA_176_5401 | 444 | 451 | 176 | 5401 |
| 223 | FETUA_346_1102 | 444 | 460 | 346 | 1102 |
| 224 | PEP-APOA1_THLAPYSDELR | 448 | 461 | N/A | N/A |
| 193 | TRFE_630_5411 | 427 | 442 | 630 | 5411 |
| 189 | AGP1_103_8704 | 418 | 439 | 103 | 8704 |
| 194 | CERU_138_6502 | 300 | 310 | 138 | 6502 |
| 184 | A1AT_107_6513 | 421 | 435 | 107 | 6513 |
| 195 | A2MG_1424_5411 | 420 | 431 | 1424 | 5411 |
| 192 | APOD_98_5411 | 426 | 441 | 98 | 5411 |
| 225 | C4BPA_221_5402 | 449 | 462 | 221 | 5402 |

### VI.E. General Methodology System for Diagnosis of Ovarian Cancer based on Table 43B

FIG. 48B is a flowchart of a process for diagnosing a subject with respect to an ovarian cancer disease state in accordance with one or more embodiments. Process 600B may be implemented using, for example, at least a portion of workflow 100 as described in FIGS. 1, 2A, and 2B and/or analysis system 300 as described in FIG. 3. Process 600B may be used to generate a final output that includes at least a diagnosis output for the subject such as, for example early stage EOC or late stage EOC.

Step 602B includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 43B, with the peptide sequence being one of SEQ ID NOS: 310, 314, 429, 430, 434, 436, 439, 442, 451, 453, 457, 465, 466, 467, 468, 469, 470, 471, 472, 473, and 474 in Table 43B, the SEQ ID NOS being defined in Table 45 below. It should be noted that the glycopeptides of Table 43B were part of a glycoprotein that are further described in Table 46 and that the glycan portion of the glycopeptides is described in Table 47.

Step 604B includes analyzing the peptide structure data using a supervised machine learning model to generate a disease indicator that predicts whether the biological sample evidences a early stage or late stage EOC on at least one peptide structures selected from a group of peptide structures identified in Table 43B. In step 604B, the group of peptide structures is associated with the early stage or late stage EOC. For example, the group of peptide structures in Table 43B includes peptide structures that have been determined relevant to distinguishing between early stage (stages 1 and 2) or late stage (stages 3 and 4) EOC.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, or all 36 of the peptide structures PS-168, PS-172, PS-182, PS-200, PS-201, PS-205, PS-220, PS-226 to PS-254 identified in Table 43B.

In one or more embodiments, step 604B may be implemented using a binary classification model (e.g., a regression model). In some examples, the regression model may be, for example, penalized multivariable regression model. In various embodiments, the disease indicator may be computed using a weight coefficient associated with each peptide structure of the at least 1 peptide structure, the weight coefficient of a corresponding peptide structure of the at least 1 peptide structures may indicate the relative significance of the corresponding peptide structure to the disease indicator.

In some embodiments, step 604B may include computing a peptide structure profile for the biological sample that identifies a weighted value for each peptide structure of the at least 1 peptide structure. The weighted value for a peptide structure of the at least 1 peptide structure may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is evidences malignancy (e.g., EOC) and the supervised machine learning model is configured to generate an output that identifies the biological sample as either evidencing (“positive for”) malignancy when the disease indicator is greater than a selected threshold or not evidencing (“negative for”) malignancy when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 606B includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, early stage or late stage based on the disease indicator. An early stage diagnosis may mean that the biological sample evidences a stage 1 or 2 EOC. A late stage diagnosis may mean that the biological sample evidences a stage 3 or 4 EOC.

Generating the diagnosis output in step 606B may include determining that the score falls above (or at or above) a selected threshold and generating a positive diagnosis for the late stage ovarian cancer disease state. Alternatively, step 606B can include determining that the score falls below (or at or below) a selected threshold and generating a negative diagnosis for the late stage ovarian cancer disease state. In some scoring systems, the score can include a probability score and the selected threshold can be 0.5. In other scoring systems, the selected threshold can fall within a range between 0.30 and 0.65.

In one or more embodiments, the final output in step 606B may include a treatment output if the disease indicator predicts malignancy and/or the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

TABLE 43B
Group of Peptide Structures Associated with Ovarian Cancer
(may be used to distinguish between early stage v. late stage ovarian cancer)

| | Peptide | | | Mono- | Linking | Linking | |
|---|---|---|---|---|---|---|---|
| | Structure | (Protein) | (Peptide) | isotopic | Site Pos. | Site Pos. | Glycan |
| PS-ID | (PS) | SEQ ID | SEQ ID | mass | in Protein | in Peptide | Structure |
| NO. | NAME | NO. | NO. | (Da) | Sequence | Sequence | GL NO. |
| 226 | AACT_106_6503 | 419 | 434 | 5260.186906 | 106 | 2 | 6503 |
| 182 | AACT_106_6513 | 419 | 434 | 5406.244812 | 106 | 2 | 6513 |
| 227 | AACT_106_7604 | 419 | 434 | 5916.414504 | 106 | 2 | 7604 |
| 219 | AACT_106_7614 | 419 | 434 | 6062.47241 | 106 | 2 | 7614 |
| 228 | AACT_271_6502 | 419 | 430 | 4321.777556 | 271 | 4 | 6502 |
| 229 | AGP1_103_6503 | 418 | 439 | 3636.3824 | 103 | 2 | 6503 |
| 230 | AGP1_103_6513 | 418 | 439 | 3782.440306 | 103 | 2 | 6513 |
| 231 | AGP1_103_7602 | 418 | 439 | 3710.419178 | 103 | 2 | 7602 |
| 232 | AGP1_103_7614 | 418 | 439 | 4438.667904 | 103 | 2 | 7614 |
| 233 | AGP1_93_6503 | 418 | 429 | 4775.889354 | 93 | 7 | 6503 |
| 234 | AGP1_93_6513 | 418 | 429 | 4921.94726 | 93 | 7 | 6513 |
| 235 | AGP1_93_7613 | 418 | 429 | 5287.079448 | 93 | 7 | 7613 |
| 201 | AGP1_93_7614 | 418 | 429 | 5578.174858 | 93 | 7 | 7614 |
| 236 | AGP12_56_6503 | 418, 463 | 465 | 3656.339868 | 56 | 5 | 6503 |
| 237 | AGP12_56_6513 | 418, 463 | 465 | 3802.397774 | 56 | 5 | 6513 |
| 238 | AGP12_72_6503 | 418, 463 | 466 | 4779.946464 | 72 | 15 | 6503 |
| 239 | AGP12_72_7603 | 418, 463 | 466 | 5145.078652 | 72 | 15 | 7603 |
| 240 | AGP2_103_6513 | 463 | 467 | 3768.424656 | 103 | 2 | 6513 |
| 241 | APOH_162_6503 | 425 | 468 | 4328.746982 | 162 | 8 | 6503 |
| 168 | CERU_138_6513 | 300 | 310 | 4898.89152 | 138 | 10 | 6513 |
| 242 | CERU_397_6513 | 300 | 469 | 5133.049472 | 397 | 2 | 6513 |
| 243 | CERU_762_6513 | 300 | 470 | 5028.0545 | 762 | 9 | 6513 |
| 244 | FETUA_156_6503 | 444 | 471 | 4631.839236 | 156 | 12 | 6503 |
| 200 | FETUA_176_6513 | 444 | 451 | 5371.203674 | 176 | 11 | 6513 |
| 245 | HEMO_187_6503 | 302 | 472 | 4264.661538 | 187 | 7 | 6503 |
| 246 | HEMO_187_6513 | 302 | 472 | 4410.719444 | 187 | 7 | 6513 |
| 247 | HPT_184_6513 | 304 | 453 | 5685.442854 | 184 | 6 | 6513 |
| 248 | HPT_207_11904 | 304 | 314 | 6232.403228 | 207 | 5, 9 | 5402 or 6502 |
| 249 | HPT_207_11915 | 304 | 314 | 6669.556544 | 207, 211 | 5, 9 | 5402 or 6513 |
| 172 | HPT_207_121005 | 304 | 314 | 6888.630826 | 207 | 5, 9 | 6502 or 6503 |
| 250 | HPT_241_6513 | 304 | 457 | 4801.061826 | 241 | 6 | 6513 |
| 251 | HPT_241_7613 | 304 | 457 | 5166.194014 | 241 | 6 | 7613 |
| 252 | KNG1_205_6513 | 464 | 473 | 4419.754828 | 205 | 9 | 6513 |
| 253 | KNG1_294_6503 | 464 | 474 | 4291.682988 | 294 | 6 | 6503 |
| 205 | TRFE_630_6513 | 427 | 442 | 5521.174696 | 630 | 9 | 6513 |
| 254 | VTNC_242_6503 | 422 | 436 | 5632.31501 | 242 | 1 | 6503 |

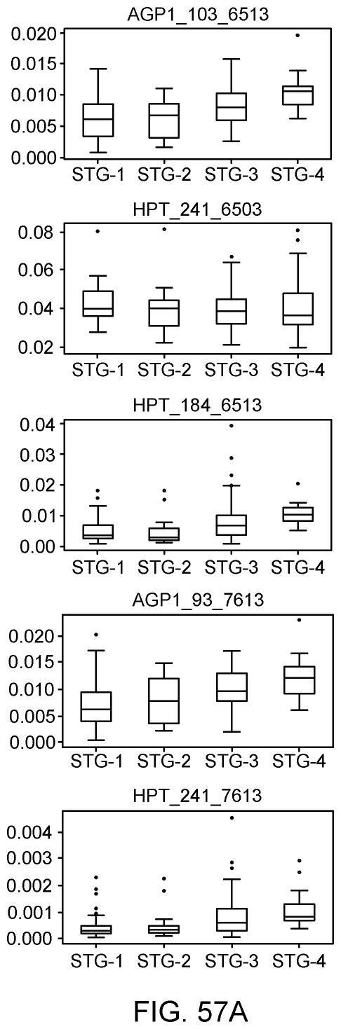

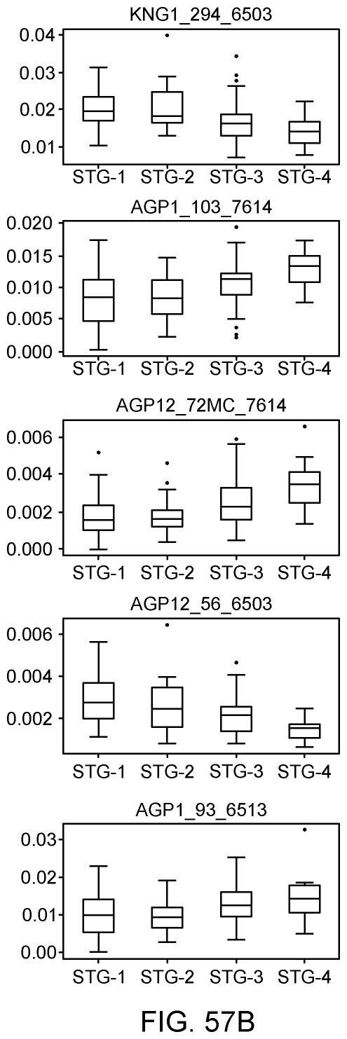

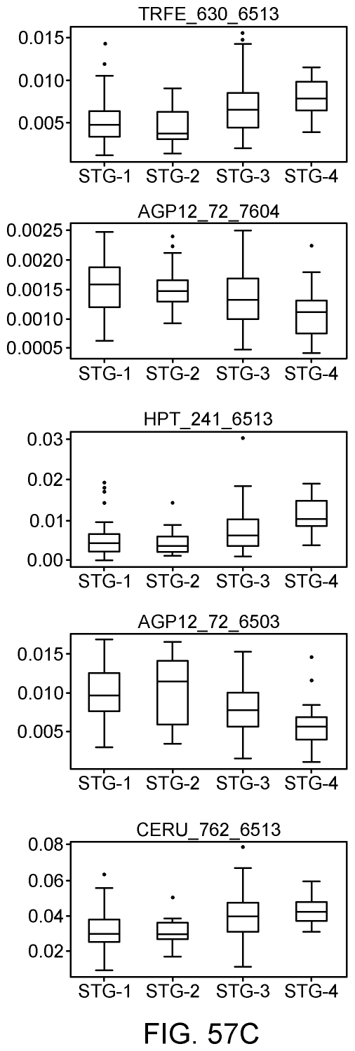

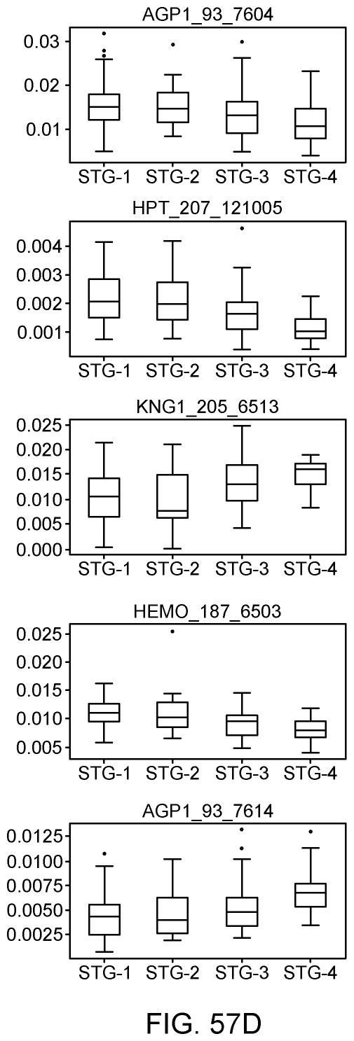

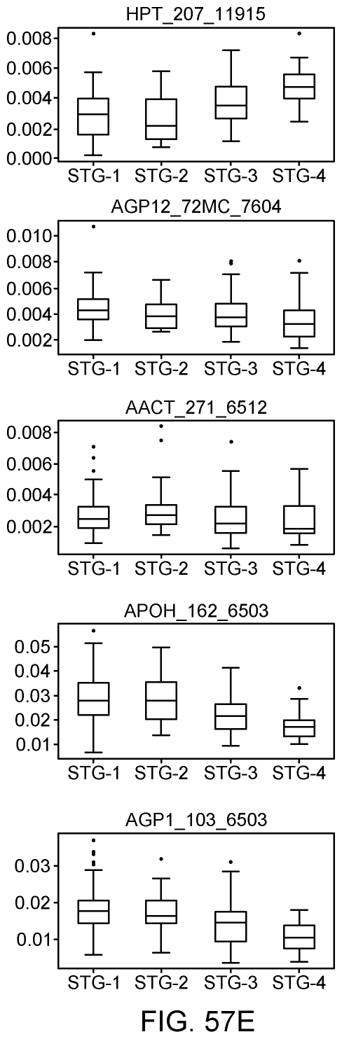

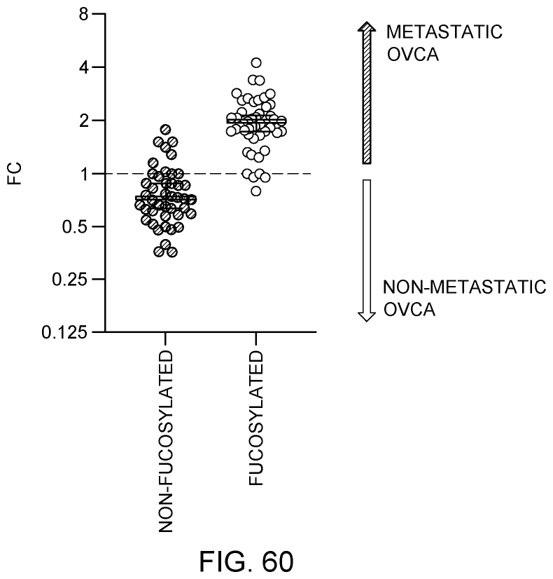

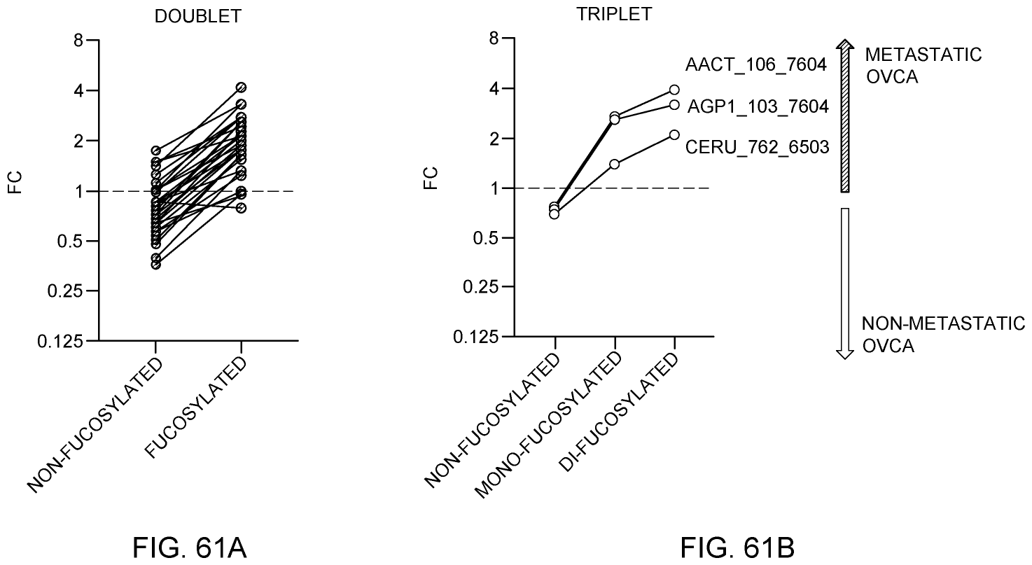

It is worthwhile to note that with a few exceptions (PS-226 and PS-231), the majority of glycopeptides were tri- and tetra-antennary glycans with or without a fucose and were found to be associated with either early stage or late stage EOC. Fold changes (FCs) for several glycopeptides in stage IV (referred to as metastatic ovarian cancer) vs benign/stage I/II/I11 (referred to as non-metastatic ovarian cancer) were calculated by normalizing to normal blood samples, as illustrated in FIG. 60. The FCs were observed to stratify between fucosylated and non-fucosylated (plots include median and 95% confidence interval). FCs that were above the 1 corresponded to markers that correlate with metastatic ovarian cancer and those below 1 corresponded to markers that correlate with non-metastatic ovarian cancer. The Wilcoxon matched-pairs signed rank test was used to compare the two groups and a p value found to be <0.0001 showing a statistical difference between non-fucosylated and fucosylated. FIGS. 61A and 61B illustrate that a same set of markers in doublets/triplets analysis for fucosylation revealed a strong association with either metastatic ovarian cancer or non-metastatic ovarian cancer. Doublet analysis refers to monitoring the fold change of a non-fucosylated and fucosylated glycopeptide that was tri or tetra-antennary for sialic acid and had the same peptide sequence and glycan linking site. Triplet analysis refers to monitoring the fold change of a non-fucosylated, fucosylated, and di-fucosylated glycopeptide that was tri or tetraantennary for sialic acid and had the same peptide sequence and glycan linking site. FIGS. 57A-57E shows that the fucosylated biomarkers (have a number 1 in the 2^{nd} to last number in the Peptide structure (PS) Name) show a relatively upward trend from stage 1/2 to stage 3/4. In contrast, FIGS. 57A-57E shows that the non-fucosylated biomarkers (have a number 0 in the 2^{nd} to last number in the Peptide structure (PS) Name) show an relatively downward trend from stage 1/2 to stage 3/4. For instance, the glycan numbers 6513, 7613, 7614 are examples of fucosylated glycans having tri or tetra-antennary sialic acids. The glycan numbers 6503, 7603, 7604 are examples of non-fucosylated glycans having tri or tetra-antennary sialic acids.

### VI.F. General Methodology System for Diagnosis of Ovarian Cancer Based on Table 43C

In another embodiment, process 600B may be implemented using Table 43C instead of Table 43B. In some cases, at least one peptide structure includes a glycopeptide structure having a peptide sequence and a glycan structure linked to the peptide sequence at a linking site of the peptide sequence, as identified in Table 43C, with the peptide sequence being one of SEQ ID NOS: 475-499 in Table 43C.

The group of peptide structures in Table 43C includes peptide structures that have been determined relevant to distinguishing between early stage (stages 1 and 2) or late stage (stages 3 and 4) EOC.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide SEQ ID NOS: 475-499 identified in Table 43C.

TABLE 43C
Group of Peptide Structures Associated with Ovarian Cancer
(may be used to distinguish between early state v. late stage ovarian cancer)

| | | | | Linking | | |
|---|---|---|---|---|---|---|
| | | | | Site | Linking | |
| | | | | Position | Site | |
| | | | | in | Position in | Glycan |
| PPS- | SEQ ID | Peptide Structure | | Protein | Peptide | Structure |
| ID NO | INO | (PS) NAME | Peptide Sequence | Sequence | Sequence | GL NO. |
| 255 | 475 | AACT_271_6512 | YTGNASALFILPDQDK | 271 | 4 | 6512 |
| 256 | 476 | AGP1_103_6503 | ENGTISR | 103 | 2 | 6503 |
| 257 | 477 | AGP1_103_6513 | ENGTISR | 103 | 2 | 6513 |
| 258 | 478 | AGP1_103_7614 | ENGTISR | 103 | 2 | 7614 |
| 259 | 479 | AGP1_93_6513 | QDQCIYNTTYLNVQR | 93 | 7 | 6513 |
| 260 | 480 | AGP1_93_7604 | QDQCIYNTTYLNVQR | 93 | 7 | 7604 |
| 261 | 481 | AGP1_93_7613 | QDQCIYNTTYLNVQR | 93 | 7 | 7613 |
| 262 | 482 | AGP1_93_7614 | QDQCIYNTTYLNVQR | 93 | 7 | 7614 |
| 263 | 483 | AGP12_56_6503 | NEEYNK | 56 | 5 | 6503 |
| 264 | 484 | AGP12_72_6503 | SVQEIQATFFYFTPNK | 72 | 15 | 6503 |
| 265 | 485 | AGP12_72_7604 | SVQEIQATFFYFTPNK | 72 | 15 | 7604 |
| 266 | 486 | AGP12_72MC_7604 | SVQEIQATFFYFTPNKT | 72 | 15 | 7604 |
| | | | EDTIFLR | | | |
| 267 | 487 | AGP12_72MC_7614 | SVQEIQATFFYFTPNKT | 72 | 15 | 7614 |
| | | | EDTIFLR | | | |
| 268 | 488 | APOH_162_6503 | VYKPSAGNNSLYR | 162 | 8 | 6503 |
| 269 | 489 | CERU_762_6513 | ELHHLQEQNVSNAFL | 762 | 9 | 6513 |
| | | | DK | | | |
| 270 | 490 | HEMO_187_6503 | SWPAVGNCSSALR | 187 | 7 | 6503 |
| 271 | 491 | HPT_184_6513 | MVSHHNLTTGATLINE | 184 | 6 | 6513 |
| | | | QWLLTTAK | | | |
| 272 | 492 | HPT_207_11915 | NLFLNHSENATAK | 207 | 5, 9 | 5402 or |
| | | | | | | 6503 |
| 273 | 493 | HPT_207_121005 | NLFLNHSENATAK | 207 | 5, 9 | 6502 or |
| | | | | | | 6503 |
| 274 | 494 | HPT_241_6503 | VVLHPNYSQVDIGLIK | 241 | 6 | 6503 |
| 275 | 495 | HPT_241_6513 | VVLHPNYSQVDIGLIK | 241 | 6 | 6513 |
| 276 | 496 | HPT_241_7613 | VVLHPNYSQVDIGLIK | 241 | 6 | 7613 |
| 277 | 497 | KNG1_205_6513 | ITYSIVQTNCSK | 205 | 9 | 6513 |
| 278 | 498 | KNG1_294_6503 | LNAENNATFYFK | 294 | 6 | 6503 |
| 279 | 499 | TRFE_630_6513 | QQQHLFGSNVTDCSG | 630 | 9 | 6513 |
| | | | NFCLFR | | | |

In Table 43C, the first three or four characters before the first underscore of the peptide structure (PS) name corresponds to the abbreviation of the protein name. More details on the protein sequence can be found in Table 46 below.

### VI.G. General Methodology System for Diagnosis of Ovarian Cancer Based on Table 43D

In another embodiment, process 600B may be implemented using Table 43D instead of Table 43B. In some cases, at least one peptide structure includes a glycopeptide structure having a peptide sequence and a glycan structure linked to the peptide sequence at a linking site of the peptide sequence, as identified in Table 43D, with the peptide sequence being one of SEQ ID NOS: 500-549 in Table 43D.

The group of peptide structures in Table 43D includes peptide structures that have been determined relevant to distinguishing between early stage (stages 1 and 2) or late stage (stages 3 and 4) EOC.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or all 50 of the peptide SEQ ID NOS: 500-549 identified in Table 43D.

TABLE 43D
Group of Peptide Structures Associated with Ovarian Cancer
(may be used to distinguish between early state v. late stage ovarian cancer)

| | | | | | Linking | Linking | | |
|---|---|---|---|---|---|---|---|---|
| PS- | | | Prot | Pep | Site Pos. | Site Pos. | Glycan | Monoisotopic |
| ID | | | SEQ ID | SEQ ID | in Protein | in Peptide | Structure | mass |
| NO. | PS-Name | Protein Name | NO. | NO. | Seq | Seq | GL NO. | (Da) |
| 280 | A1AT_107_6512 | Alpha-1- | 421 | 500 | 107 | 14 | 6512 | 6406.779 |
| | | antitrypsin | | | | | | |
| 281 | A2MG_1424_6501 | Alpha-2- | 420 | 501 | 1424 | 3 | 6501 | 4440.983 |
| | | macroglobulin | | | | | | |
| 282 | A2MG_1424_6511 | Alpha-2- | 420 | 502 | 1424 | 3 | 6511 | 4587.041 |
| | | macroglobulin | | | | | | |
| 283 | AACT_106_6503 | Alpha-1- | 419 | 503 | 106 | 2 | 6503 | 5260.187 |
| | | antichymotrypsin | | | | | | |
| 284 | AACT_106_7604 | Alpha-1- | 419 | 504 | 106 | 2 | 7604 | 5916.415 |
| | | antichymotrypsin | | | | | | |
| 285 | AACT_106_7614 | Alpha-1- | 419 | 505 | 106 | 2 | 7614 | 6062.472 |
| | | antichymotrypsin | | | | | | |
| 286 | AACT_271_6502 | Alpha-1- | 419 | 506 | 271 | 4 | 6502 | 4321.778 |
| | | antichymotrypsin | | | | | | |
| 287 | AACT_271_6503 | Alpha-1- | 419 | 507 | 271 | 4 | 6503 | 4612.873 |
| | | antichymotrypsin | | | | | | |
| 288 | AGP12_56_6503 | Alpha-1-acid | 418 or 463 | 508 | 56 | 5 | 6503 | 3656.34 |
| | | glycoprotein 1 | | | | | | |
| | | and/or 2 | | | | | | |
| 289 | AGP12_72MC_6503 | Alpha-1-acid | 418 or 463 | 509 | 72 | 15 | 6503 | 5755.449 |
| | | glycoprotein 1 | | | | | | |
| | | and/or 2 | | | | | | |
| 290 | AGP12_72MC6513 | Alpha-1-acid | 418 or 463 | 510 | 72 | 15 | 6513 | 5901.507 |
| | | glycoprotein 1 | | | | | | |
| | | and/or 2 | | | | | | |
| 291 | AGP12_72MC_7613 | Alpha-1-acid | 418 or 463 | 511 | 72 | 15 | 7613 | 6266.639 |
| | | glycoprotein 1 | | | | | | |
| | | and/or 2 | | | | | | |
| 292 | AGP12_72MC7614 | Alpha-1-acid | 418 or 463 | 512 | 72 | 15 | 7614 | 6557.734 |
| | | glycoprotein 1 | | | | | | |
| | | and/or 2 | | | | | | |
| 293 | AGP12_72_6503 | Alpha-1-acid | 418 or 463 | 513 | 72 | 15 | 6503 | 4779.946 |
| | | glycoprotein 1 | | | | | | |
| | | and/or 2 | | | | | | |
| 294 | AGP12_72_7603 | Alpha-1-acid | 418 or 463 | 514 | 72 | 15 | 7603 | 5145.079 |
| | | glycoprotein 1 | | | | | | |
| | | and/or 2 | | | | | | |
| 295 | AGP12_72_7604 | Alpha-1-acid | 418 or 463 | 515 | 72 | 15 | 7604 | 5436.174 |
| | | glycoprotein 1 | | | | | | |
| | | and/or 2 | | | | | | |
| 296 | AGP1_103_6503 | Alpha-1-acid | 418 | 516 | 103 | 2 | 6503 | 3636.382 |
| | | glycoprotein 1 | | | | | | |
| 297 | AGP1_103_6513 | Alpha-1-acid | 418 | 517 | 103 | 2 | 6513 | 3782.44 |
| | | glycoprotein 1 | | | | | | |
| 298 | AGP1_103_7602 | Alpha-1-acid | 418 | 518 | 103 | 2 | 7602 | 3710.419 |
| | | glycoprotein 1 | | | | | | |
| 299 | AGP1_103_7604 | Alpha-1-acid | 418 | 519 | 103 | 2 | 7604 | 4292.61 |
| | | glycoprotein 1 | | | | | | |
| 300 | AGP1_103_7614 | Alpha-1-acid | 418 | 520 | 103 | 2 | 7614 | 4438.668 |
| | | glycoprotein 1 | | | | | | |
| 301 | AGP1_93_6503 | Alpha-1-acid | 418 | 521 | 93 | 7 | 6503 | 4775.889 |
| | | glycoprotein 1 | | | | | | |
| 302 | AGP1_93_7602 | Alpha-1-acid | 418 | 522 | 93 | 7 | 7602 | 4849.926 |
| | | glycoprotein 1 | | | | | | |
| 303 | AGP1_93_7603 | Alpha-1-acid | 418 | 523 | 93 | 7 | 7603 | 5141.022 |
| | | glycoprotein 1 | | | | | | |
| 304 | AGP1_93_7604 | Alpha-1-acid | 418 | 524 | 93 | 7 | 7604 | 5432.117 |
| | | glycoprotein 1 | | | | | | |
| 305 | AGP1_93_7612 | Alpha-1-acid | 418 | 525 | 93 | 7 | 7612 | 4995.984 |
| | | glycoprotein 1 | | | | | | |
| 306 | AGP1_93_7613 | Alpha-1-acid | 418 | 526 | 93 | 7 | 7613 | 5287.079 |
| | | glycoprotein 1 | | | | | | |
| 307 | AGP1_93_7614 | Alpha-1-acid | 418 | 527 | 93 | 7 | 7614 | 5578.175 |
| | | glycoprotein 1 | | | | | | |
| 308 | AGP2_103_6503 | Alpha-1-acid | 463 | 528 | 103 | 2 | 6503 | 3622.367 |
| | | glycoprotein 2 | | | | | | |
| 309 | AGP2_103_6513 | Alpha-1-acid | 463 | 529 | 103 | 2 | 6513 | 3768.425 |
| | | glycoprotein 2 | | | | | | |
| 310 | AGP2_103_7604 | Alpha-1-acid | 463 | 530 | 103 | 2 | 7604 | 4278.594 |
| | | glycoprotein 2 | | | | | | |
| 311 | APOH_162_6503 | Beta-2- | 425 | 531 | 162 | 8 | 6503 | 4328.747 |
| | | glycoprotein1 | | | | | | |
| 312 | APOH_253_5412 | Beta-2- | 425 | 532 | 253 | 3 | 5412 | 3600.389 |
| | | glycoprotein1 | | | | | | |
| 313 | CERU_397_6513 | Ceruloplasmin | 300 | 533 | 397 | 2 | 6513 | 5133.049 |
| 314 | CERU_762_6503 | Ceruloplasmin | 300 | 534 | 762 | 9 | 6503 | 4881.997 |
| 315 | FETUA_156_6503 | Alpha-2-HS- | 444 | 535 | 156 | 12 | 6503 | 4631.839 |
| | | glycoprotein | | | | | | |
| 316 | FETUA_156_6513 | Alpha-2-HS- | 444 | 536 | 156 | 12 | 6513 | 4777.897 |
| | | glycoprotein | | | | | | |
| 317 | FETUA_176_6513 | Alpha-2-HS- | 444 | 537 | 176 | 11 | 6513 | 5371.204 |
| | | glycoprotein | | | | | | |
| 318 | HEMO_187_6503 | Hemopexin | 302 | 538 | 187 | 7 | 6503 | 4264.662 |
| 319 | HEMO_187_6513 | Hemopexin | 302 | 539 | 187 | 7 | 6513 | 4410.719 |
| 320 | HPT_184_6513 | Haptoglobin | 304 | 540 | 184 | 6 | 6513 | 5685.443 |
| 321 | HPT_207_11904 | Haptoglobin | 304 | 541 | 207 or 211 | 5 or 9 | 5402 or 6502 | 6232.403 |
| 322 | HPT_207_11914 | Haptoglobin | 304 | 542 | 207 or 211 | 5 or 9 | 5402 or 6512 | 6378.461 |
| 323 | HPT_207_11915 | Haptoglobin | 304 | 543 | 207 or 211 | 5 or 9 | 5402 or 6513 | 6669.557 |
| 324 | HPT_207_121005 | Haptoglobin | 304 | 544 | 207 or 211 | 5 or 9 | 6502 or 6503 | 6888.631 |
| 325 | HPT_241_6511 | Haptoglobin | 304 | 545 | 241 | 6 | 6511 | 4218.871 |
| 326 | HPT_241_6512 | Haptoglobin | 304 | 546 | 241 | 6 | 6512 | 4509.966 |
| 327 | HPT_241_6513 | Haptoglobin | 304 | 547 | 241 | 6 | 6513 | 4801.062 |
| 328 | HPT_241_7613 | Haptoglobin | 304 | 548 | 241 | 6 | 7613 | 5166.194 |
| 329 | KNG1_294_6503 | Kininogen-1 | 464 | 549 | 294 | 6 | 6503 | 4291.683 |

With respect to Tables 41, 42, 43A, 43B, 43C, and 43D, they include the Peptide Structure (PS) Name (e.g., KNG1_294_6503), which is a reference code for the protein name (e.g., KNG1), followed by the glycan linking site position in the protein (e.g., the number 294 that is preceded by an underscore and represents a sequential amino acid position in protein KNG1), and followed by the glycan structure GL number (e.g., the number 6503 that is preceded by an underscore and represents a glycan composition Hex(6)HexNAc(5)Fuc(0)NeuAc(3)). The Peptide Structure (PS) Name of contains a prefix that represents an abbreviation (that may include a combination of letters and numbers) for a protein abbreviation that corresponds to the Protein Abbreviation of Table 46. The term Linking Site Pos. in Protein Sequence is a number that refers to the sequential position of an amino acid of the corresponding protein in which a glycan is attached. For the Linking Site Pos. in Protein Sequence, the amino acid position of the peptide sequence is defined by the sequentially numbered order of amino acids based on the Uniprot ID of the corresponding protein for the peptide sequence. The term Linking Site Pos. in Peptide Sequence is a number that refers to the sequential position of an amino acid of the corresponding peptide in which a glycan is attached. For the Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Table 47. In some embodiments, the term AGP12 for SEQ ID NOs: 465-466 represent that the glycopeptide is a fragment of either AGP1 or AGP2.

In some instances, if the first number subsequent to the first underscore in the Peptide Structure (PS) NAME is inconsistent with the Glycan Linking Site Pos. in Protein Sequence column, then the Glycan Linking Site Pos. in Protein Sequence column should be used for identification of the peptide. In some instances, if the second number subsequent to the second underscore in the Peptide Structure (PS) NAME is inconsistent with the Glycan Structure GL NO column, then the Glycan Structure GL NO column should be used for identification of the glycan portion of the glycopeptide. If the Peptide Structure (PS) NAME does not contain any numbers, then the peptide is non-glycosylated. In some instances of the Peptide Structure (PS) NAME, subsequent to the prefix, there is a number noted with the notation MC that indicates that there was a missed cleavage at position in the peptide sequence as noted by the number.

### VI.H. Training a Model

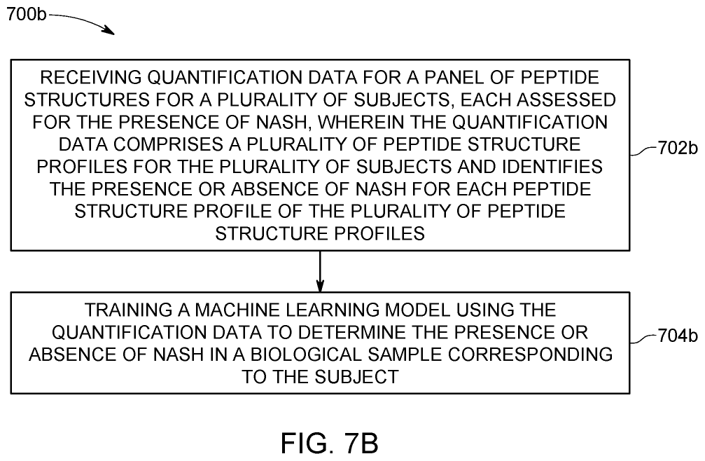

FIG. 7A illustrates and example flowchart of a process 700a for training a model to diagnose a subject with one of a plurality of states associated with non-alcoholic steatohepatitis (NASH) progression, in accordance with various embodiments. Process 700a may be implemented using, for example, analysis system 300 as described in FIG. 3. In various embodiments, process 700a may be performed to train model 314 in FIG. 3.

Step 710a includes receiving quantification data for a panel of peptide structures for a plurality of subjects, each diagnosed with one of the plurality of states associated with NASH progression, wherein the quantification data comprises a plurality of peptide structure profiles for the plurality of subjects and identifies a corresponding state of the plurality of states for each peptide structure profile of the plurality of peptide structure profiles.

In various embodiments, the quantification data for the panel of peptide structures for the plurality of subjects diagnosed with the plurality of states comprises at least one of an abundance, a relative abundance, a normalized abundance, a relative quantity, an adjusted quantity, a normalized quantity, a relative concentration, an adjusted concentration, or a normalized concentration. In various embodiments, the quantification data for the panel of peptide structures for the plurality of subjects diagnosed with the plurality of states comprises normalized abundances.

Step 720a includes training a machine learning model using the quantification data to determine which state of the plurality of states a biological sample from the subject corresponds. In various embodiments, the machine learning model comprises a logistic regression model. In various embodiments, the logistic regression model may be a LASSO regularization model or a logistic regression with Ridge regularization.

The plurality of states may include, for example, at least one of a, early stage non-alcoholic steatohepatitis (NASH) state, a late stage NASH state, or a healthy state.

In various embodiments, the training the machine learning model comprises: training the machine learning model using a portion of the quantification data corresponding to a set of peptide structures that is a subset of the panel of peptide structures to determine which state of the plurality of states the biological sample from the subject corresponds.

In various embodiments, process 700a can further include identifying the set of peptide structures as the subset of the plurality of peptide structures relevant to the determining which state of the plurality of states the biological sample from the subject corresponds based on at least one of fold-changes, false discovery rates, or p-values computed as part of the differential expression analysis.

In various embodiments, process 700a can further include performing a differential expression analysis using the quantification data for the plurality of subjects. In various embodiments, process 700a can further include identifying the set of peptide structures as the subset of the plurality of peptide structures relevant to the determining which state of the plurality of states the biological sample from the subject corresponds based on at least one of fold-changes, false discovery rates, or p-values computed as part of the differential expression analysis.

In various embodiments, process 700a can further include determining normalized abundance for the panel of peptide structures based on a relative abundance of the peptide structures and an average relative raw abundance of peptide structure in a reference serum sample. In various embodiments, process 700a can further include determining relative abundance based on a raw abundance of the peptide structures and a raw abundance of a glycosylated peptide from a common glycoprotein in the panel.

Turning attention to FIG. 7B, an example flowchart of a process 700b is provided for training a model to detect the presence of non-alcoholic steatohepatitis (NASH) in a subject, in accordance with various embodiments. Process 700b may be implemented using, for example, analysis system 300 as described in FIG. 3. In various embodiments, process 700b may be performed to train model 314 in FIG. 3.

Step 710b includes receiving quantification data for a panel of peptide structures for a plurality of subjects, each assessed for the presence of NASH, wherein the quantification data comprises a plurality of peptide structure profiles for the plurality of subjects and identifies the presence or absence of NASH for each peptide structure profile of the plurality of peptide structure profiles.

In various embodiments, the quantification data for the panel of peptide structures for the plurality of subjects assessed for the presence of NASH, comprises at least one of an abundance, a relative abundance, a normalized abundance, a relative quantity, an adjusted quantity, a normalized quantity, a relative concentration, an adjusted concentration, or a normalized concentration. In various embodiments, the quantification data for the panel of peptide structures for the plurality of subjects assessed for the presence of NASH, comprises normalized abundances.

Step 720b includes training a machine learning model using the quantification data to determine the presence or absence of NASH in a biological sample corresponding to the subject. In various embodiments, the machine learning model comprises a logistic regression model. In various embodiments, the logistic regression model may be a LASSO regularization model or a logistic regression with Ridge regularization.

In various embodiments, the training the machine learning model can include training the machine learning model using a portion of the quantification data corresponding to a set of peptide structures that is a subset of the panel of peptide structures to determine the presence or absence of NASH in a biological sample corresponding to the subject.

In various embodiments, process 700b can further include identifying the set of peptide structures as the subset of the plurality of peptide structures relevant to the determining the presence or absence of NASH in a biological sample corresponding to the subject, based on at least one of fold-changes, false discovery rates, or p-values computed as part of the differential expression analysis.

In various embodiments, process 700b can further include performing a differential expression analysis using the quantification data for the plurality of subjects. In various embodiments, process 700b can further include identifying the set of peptide structures as the subset of the plurality of peptide structures relevant to the determining which state of the plurality of states the biological sample from the subject corresponds based on at least one of fold-changes, false discovery rates, or p-values computed as part of the differential expression analysis.

In various embodiments, process 700b can further include determining normalized abundance for the panel of peptide structures based on a relative abundance of the peptide structures and an average relative raw abundance of peptide structure in a reference serum sample. In various embodiments, process 700b can further include determining relative abundance based on a raw abundance of the peptide structures and a raw abundance of a glycosylated peptide from a common glycoprotein in the panel.

The peptide structures selected for training may be peptide structures identified from, for example, a differential expression analysis performed using initial training data to compare the quantification data for peptide structures for a first portion of the plurality of subjects diagnosed with early stage NASH, a second portion of the plurality of subjects diagnosed with late stage NASH, and a third portion of the plurality of subjects diagnosed with a non-NASH condition (e.g., a healthy state, a benign hepatic mass, liver disease-free state, etc.).

For example, comparisons may be performed to compare peptide structure quantification data between the first portion of subjects with early stage NASH versus the third portion of subjects with the non-NASH condition (e.g., the control state), to compare the second portion of subjects with late stage NASH versus the first portion of subjects with early stage NASH, and/or to compare the second portion of subjects with late stage NASH versus the third portion of subjects with the non-NASH condition. In various embodiments, comparisons may be performed to compare peptide structure quantification data between those subjects having NASH (e.g., the first and second portions of subjects) with the third portion of subjects with the non-NASH condition In some embodiments, the comparisons may be normalized and compared to one another.

In various embodiments, the biological sample can include at least one of blood, serum, or plasma. In various embodiments, the quantification data is generated using a liquid chromatography/mass spectrometry (LC/MS) system. In various embodiments, the quantification data is generated using multiple reaction monitoring mass spectrometry (MRM-MS).

Tables 2A-2B and 3A-3B below indicate the respective fold changes (FC), false discovery rates (FDR), and p-values computed based on such comparisons.

TABLE 2A
Various embodiments of Differential Expression Analysis for NASH v.
Control

| PS-ID | PS- | NASH/Control | NASH/Control | NASH/Control |
|---|---|---|---|---|
| NO. | NAME | (fold change) | (FDR) | (p-value) |
| PS-01 | A1AT_271_5402 | 1.054312229 | 4.13E-05 | 0.000120543 |
| PS-02 | A2MG_1424_5402 | 1.238837044 | 9.45E-09 | 5.38E-08 |
| PS-03 | A2MG_1424_NONGLYCOSYL | 0.851126574 | 6.93E-11 | 6.24E-10 |
| | ATED | | | |
| PS-04 | AACT_271_7602 | 0.768626602 | 0.003600496 | 0.007221805 |
| PS-05 | AGP12_72_7604 | 0.675408636 | 2.90E-16 | 5.02E-15 |
| PS-06 | APOB_3895_5401 | 0.91828099 | 0.023279651 | 0.04059316 |
| PS-07 | APOC3_74_1101 | 0.904569791 | 0.000102696 | 0.000276245 |
| PS-08 | HRG_271_2202 | 1.255244922 | 6.36E-11 | 5.81E-10 |
| PS-09 | IGA12_144_4401 | 0.879950635 | 0.039833377 | 0.06550797 |
| PS-10 | IGG1_297_5410 | 0.910938596 | 0.017424647 | 0.031006936 |
| PS-11 | IGG2_297_4411 | 1.086504313 | 0.00304694 | 0.006164945 |
| PS-12 | IGG2_297_5410 | 1.25183147 | 3.31E-05 | 9.86E-05 |
| PS-13 | KLKB1_494MC_5402 | 1.347101383 | 5.60E-05 | 0.000156739 |
| PS-14 | ANT3_FATTFYQHLADSK | 0.843303121 | 0.000130833 | 0.000341346 |
| PS-15 | FETUA_346_NONGLYCOSYL | 0.938853736 | 0.008158095 | 0.015427025 |
| | ATED | | | |
| PS-16 | HEMO_187_5401 | 1.008809693 | 0.723607817 | 0.776172837 |
| PS-17 | APOB_983_5402 | 1.126456927 | 2.57E-08 | 1.35E-07 |
| PS-18 | AGP1_93_7604 | 1.17642544 | 0.026191362 | 0.047054137 |
| PS-19 | KLKB1_453_5402 | 0.861510837 | 5.77E-05 | 0.000199229 |
| PS-20 | CO6_324_5402 | 0.92555625 | 0.038505178 | 0.064713542 |
| PS-21 | THRB_121_5412 | 0.556950625 | 3.54E-08 | 2.56E-07 |
| PS-22 | CFAH_529_5402 | 0.767362395 | 1.39E-10 | 1.47E-09 |
| PS-23 | APOD_98_5402 | 1.120941892 | 0.000689412 | 0.001799082 |
| PS-24 | IGG2_297_3500 | 0.366802997 | 4.83E-14 | 7.36E-13 |
| PS-25 | IGG2_297_4500 | 1.005406074 | 0.895720719 | 0.919864134 |
| PS-26 | FHR1_INHGILYDEEK | 1.001651132 | 0.978163586 | 0.985262015 |
| PS-27 | APOC3_74MC_1102 | 0.963939826 | 0.285565799 | 0.361647198 |
| PS-28 | AGP1_93_6503 | 1.079755012 | 0.073575991 | 0.113977093 |
| PS-29 | PLASMAFGA_DSHSLTTNIM | 0.778306506 | 0.000109711 | 0.000292843 |
| | EILR | | | |

TABLE 2B
Various embodiments of Differential Expression Analysis for NASH v.
Control

| PS-ID | | NASH/Control | NASH/Control | NASH/Control |
|---|---|---|---|---|
| NO. | PS-NAME | (fold change) | (FDR) | (p-value) |
| PS-01 | A1AT_271_5402 | 1.054312229 | 4.13E-05 | 0.000120543 |
| PS-02 | A2MG_1424_5402 | 1.238837044 | 9.45E-09 | 5.38E-08 |
| PS-03 | A2MG_1424_NONGLYCOSYL | 0.851126574 | 6.93E-11 | 6.24E-10 |
| | ATED | | | |
| PS-04 | AACT_271_7602 | 0.768626602 | 0.003600496 | 0.007221805 |
| PS-05 | AGP12_72_7604 | 0.675408636 | 2.90E-16 | 5.02E-15 |
| PS-06 | APOB_3895_5401 | 0.91828099 | 0.023279651 | 0.04059316 |
| PS-07 | APOC3_74_1101 | 0.904569791 | 0.000102696 | 0.000276245 |
| PS-08 | HRG_271_2202 | 1.255244922 | 6.36E-11 | 5.81E-10 |
| PS-09 | IGA12_144_4401 | 0.879950635 | 0.039833377 | 0.06550797 |
| PS-10 | IGG1_297_5410 | 0.910938596 | 0.017424647 | 0.031006936 |
| PS-11 | IGG2_297_4411 | 1.086504313 | 0.00304694 | 0.006164945 |
| PS-12 | IGG2_297_5410 | 1.25183147 | 3.31E-05 | 9.86E-05 |
| PS-13 | KLKB1_494MC_5402 | 1.347101383 | 5.60E-05 | 0.000156739 |
| PS-14 | ANT3_FATTFYQHLADSK | 0.843303121 | 0.000130833 | 0.000341346 |

TABLE 3A
Various embodiments of Differential Expression Analysis for NASH (early)
vs. NASH (late)

| | | NASH(early)/ | NASH(early)/ | NASH(early)/ |
|---|---|---|---|---|
| PS-ID | | NASH(late) | NASH(late) | NASH(late) |
| NO. | PS-NAME | (fold change) | (p-value) | (FDR) |
| PS-01 | A1AT_271_5402 | 1.066606018 | 0.001372478 | 0.476249955 |
| PS-02 | A2MG_1424_5402 | 1.117846319 | 0.011263425 | 0.55644186 |
| PS-03 | A2MG_1424_ | 1.109923799 | 0.009906059 | 0.55644186 |
| | NONGLYCOSYLATED | | | |
| PS-04 | AACT_271_7602 | 1.521697843 | 0.005746988 | 0.55644186 |
| PS-05 | AGP12_72_7604 | 0.80400926 | 0.006567336 | 0.55644186 |
| PS-06 | APOB_3895_5401 | 1.185989144 | 0.020300622 | 0.55644186 |
| PS-07 | APOC3_74_1101 | 1.125949872 | 0.013376141 | 0.55644186 |
| PS-08 | HRG_271_2202 | 0.883300505 | 0.0301975 | 0.626007905 |
| PS-09 | IGA12_144_4401 | 0.798483588 | 0.016073703 | 0.55644186 |
| PS-10 | IGG1_297_5410 | 1.141726136 | 0.04043055 | 0.626007905 |
| PS-11 | IGG2_297_4411 | 1.09635526 | 0.014837011 | 0.55644186 |
| PS-12 | IGG2_297_5410 | 1.264256382 | 0.006321671 | 0.55644186 |
| PS-13 | KLKB1_494MC_5402 | 0.708348357 | 0.022450104 | 0.55644186 |
| PS-14 | ANT3_ | 0.831868046 | 0.02692621 | 0.622892994 |
| | FATTFYQHLADSK | | | |
| PS-15 | FETUA_346_ | 1.002306078 | 0.945461457 | 0.98419818 |
| | NONGLYCOSYLATED | | | |
| PS-16 | HEMO_187_5401 | 0.94387067 | 0.283596853 | 0.932025086 |
| PS-17 | APOB_983_5402 | 1.042027243 | 0.273233337 | 0.932025086 |
| PS-18 | AGP1_93_7604 | 0.948432478 | 0.698954096 | 0.999930262 |
| PS-19 | KLKB1_453_5402 | 1.109708091 | 0.132280288 | 0.940464559 |
| PS-20 | CO6_324_5402 | 0.924273366 | 0.257441751 | 0.999930262 |
| PS-21 | THRB_121_5412 | 1.205754957 | 0.355266209 | 0.999930262 |
| PS-22 | CFAH_529_5402 | 1.022835534 | 0.753980296 | 0.999930262 |
| PS-23 | APOD_98_5402 | 0.952188995 | 0.441535899 | 0.990760008 |
| PS-24 | IGG2_297_3500 | 0.908028279 | 0.501329086 | 0.990760008 |
| PS-25 | IGG2_297_4500 | 1.082731843 | 0.339975891 | 0.990760008 |
| PS-26 | FHR1_INHGILYDEEK | 0.842506689 | 0.064337175 | 0.731967199 |
| PS-27 | APOC3_74MC_1102 | 0.814902387 | 0.000344328 | 0.238963329 |
| PS-28 | AGP1_93_6503 | 1.20277365 | 0.013358017 | 0.55644186 |
| PS-29 | PLASMAFGA_ | 1.030093046 | 0.672077502 | 0.98419818 |
| | DSHSLTTNIMEILR | | | |

TABLE 3B
Various embodiments of Differential Expression Analysis for NASH (early)
vs. NASH (late)

| | | NASH(early)/ | NASH(early)/ | NASH(early)/ |
|---|---|---|---|---|
| PS-ID | | NASH(late) | NASH(late) | NASH(late) |
| NO. | PS-NAME | (fold change) | (p-value) | (FDR) |
| PS-01 | A1AT_271_5402 | 1.066606018 | 0.001372478 | 0.476249955 |
| PS-02 | A2MG_1424_5402 | 1.117846319 | 0.011263425 | 0.55644186 |
| PS-03 | A2MG_1424_ | 1.109923799 | 0.009906059 | 0.55644186 |
| | NONGLYCOSYLATED | | | |
| PS-04 | AACT_271_7602 | 1.521697843 | 0.005746988 | 0.55644186 |
| PS-05 | AGP12_72_7604 | 0.80400926 | 0.006567336 | 0.55644186 |
| PS-06 | APOB_3895_5401 | 1.185989144 | 0.020300622 | 0.55644186 |
| PS-07 | APOC3_74_1101 | 1.125949872 | 0.013376141 | 0.55644186 |
| PS-08 | HRG_271_2202 | 0.883300505 | 0.0301975 | 0.626007905 |
| PS-09 | IGA12_144_4401 | 0.798483588 | 0.016073703 | 0.55644186 |
| PS-10 | IGG1_297_5410 | 1.141726136 | 0.04043055 | 0.626007905 |
| PS-11 | IGG2_297_4411 | 1.09635526 | 0.014837011 | 0.55644186 |
| PS-12 | IGG2_297_5410 | 1.264256382 | 0.006321671 | 0.55644186 |
| PS-13 | KLKB1_494MC_5402 | 0.708348357 | 0.022450104 | 0.55644186 |
| PS-14 | ANT3_ | 0.831868046 | 0.02692621 | 0.622892994 |
| | FATTFYQHLADSK | | | |

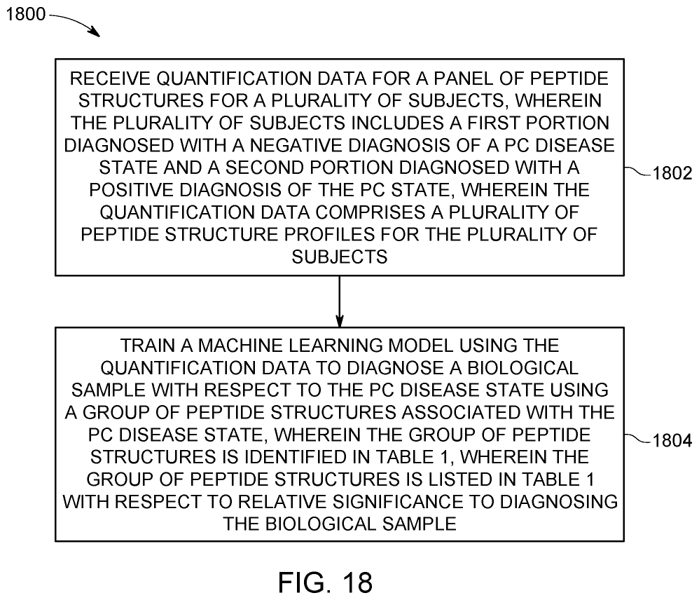

FIG. 18 is a flowchart of a process for training a model to diagnose a subject with respect to a pancreatic cancer (PC) disease state in accordance with one or more embodiments. Process 1800 may be implemented using, for example, at least a portion of workflow 100 as described in FIGS. 1, 2A, and 2B and/or analysis system 300 as described in FIG. 3. In some embodiments, process 1800 may be one example of an implementation for training the model used in the process 1700 in FIG. 17.

Step 1802 includes receiving quantification data for a panel of peptide structures for a plurality of subjects. The plurality of subjects includes a first portion diagnosed with a negative diagnosis of a PC disease state and a second portion diagnosed with a positive diagnosis of the PC disease state. The quantification data comprises a plurality of peptide structure profiles for the plurality of subjects.

Step 1804 includes training a machine learning model using the quantification data to diagnose a biological sample with respect to the PC disease state using a group of peptide structures associated with the PC disease state (e.g., the group of peptide structures is identified in Table 16). The group of peptide structures is listed in Table 16 with respect to relative significance to diagnosing the biological sample. Step 1804 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the PC disease state and a negative diagnosis for any subject of the plurality of subjects determined not to have the PC disease state.

The machine learning model can include a binary classification model. Some binary classification models can include logistical regression models. Some logistical regression models can include LASSO regression models.

An alternative or additional step in process 1800 can include performing a differential expression analysis using initial training data to compare a first portion of the plurality of subjects diagnosed with the positive diagnosis for the PC disease state versus a second portion of the plurality of subjects diagnosed with the negative diagnosis for the PC disease state.

An alternative or additional step in process 1800 can include identifying a training group of peptide structures based on the differential expression analysis for use as prognostic markers for the PC disease state.

An alternative or additional step in process 1800 can include forming the training data based on the training group of peptide structures identified.

An alternative or additional step in process 1800 can include identifying a training group of peptide structures based on the differential expression analysis, wherein the training group of peptide structures is a subset of the plurality of peptide structures relevant to diagnosing the PC disease state. The subset may be identified based on at least one of fold-changes, false discovery rates, or p-values computed as part of the differential expression analysis.

An alternative or additional step in process 1800 can include training a machine learning model, using the quantification data for the training group of peptide structures, to diagnose a subject of a biological sample with respect to the PC disease state using a group of peptide structures associated with the PC disease state. The group of peptide structures may be a subset of the training group of peptide structures and is identified in Table 16. The group of peptide structures is listed in Table 16 with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures of Table 17A, 17B, or 17C below, which include at least a portion of the group of peptide structures identified in Table 16. The markers used for training of the LASSO regression model may, in one or more embodiments, additionally include one or more other peptide structure markers.

In one or more embodiments, a subset of the markers identified in Table 17A, 17B, or 17C may be used for training of the LASSO regression model. Alternatively, the markers identified in Table 17 may be a subset for training of the LASSO regression model. For example, the LASSO regression model may be trained using at least one other marker in addition to those identified in Table 17.

### VI.I. Training a Model for Diagnosis of NSCLC

FIG. 36 illustrates a flow diagram 3600 of a method for training a model to diagnose a subject with one of a plurality of states associated with NSCLC, in accordance with the presently disclosed embodiments. The flow diagram 3600 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 3600 may begin at block 3602 with one or more processing devices (e.g., computing platform 302) receiving quantification data for a panel of peptide structures for a plurality of subjects diagnosed with the plurality of states associated with the NSCLC, wherein the quantification data comprises a plurality of peptide structure profiles for the plurality of subjects and identifies a corresponding state of the plurality of states for each peptide structure profile of the plurality of peptide structure profiles. The flow diagram 3600 may then conclude at block 3604 with one or more processing devices (e.g., computing platform 302) training a machine-learning model to determine a state of the plurality of states a biological sample from the subject corresponds based on the quantification data.

In some embodiments, the machine-learning model includes a regularized regression model (e.g., LASSO regression model) trained and evaluated, for example, over one or more iterations using a first set of peptide structure coefficients set forth in Table 39 for all stages of NSCLC (stages 1-4), using a second set of peptide structure coefficients set forth in Table 39 for early-stage NSCLC (stages 1-2), and using a third set of peptide structure coefficients set forth in Table 39 for late-stage NSCLC (stages 3-4).

### VI.J. Training a Model to Predict Ovarian Cancer (e.g., Epithelial Ovarian Cancer)

FIG. 49 is a flowchart of a process for training a model to diagnose a subject with respect to an ovarian cancer disease state in accordance with one or more embodiments. Process 700 may be implemented using, for example, at least a portion of workflow 100 as described in FIGS. 1, 2A, and 2B and/or analysis system 300 as described in FIG. 3. In some embodiments, process 700 may be one example of an implementation for training the model used in the process 500 in FIGS. 47, 48A, or 48B.

Step 702 includes receiving quantification data for a panel of peptide structures for a plurality of subjects. The plurality of subjects may include a first portion diagnosed with a negative diagnosis of an ovarian cancer disease state and a second portion diagnosed with a positive diagnosis of the ovarian cancer disease state. The plurality of subjects may include a first portion having early stage EOC and a second portion have late stage EOC. The quantification data comprises an initial plurality of peptide structure profiles for the plurality of subjects. For example, a peptide structure profile in the initial plurality of peptide structure profiles may include a feature associated with a corresponding peptide structure. The feature may be relative abundance, concentration, site occupancy, or some other quantification-based feature. The initial plurality of peptide structure profiles may include, one, two, three, or more profiles for a given peptide structure.

Step 704 includes training a machine learning model using the quantification data to diagnose a biological sample with respect to the ovarian cancer disease state using a group of peptide structures associated with the ovarian cancer disease state (e.g., the first group of peptide structures is identified in Table 41, the second group of peptide structures is identified in Table 42, the third group of peptide structures is identified in Table 43A). The first, second, and third groups of peptide structures are listed in Tables 41, 42, and 43A, respectively, with respect to relative significance to diagnosing the biological sample as evidencing malignancy (e.g., EOC). Step 704 can include training the machine learning using a portion of the quantification data corresponding to a training group of peptide structures included in the plurality of peptide structures. Step 704 can include training a machine learning model using the quantification data to assess a biological sample with respect to the staging of the ovarian cancer disease state using a group of peptide structures associated with the ovarian cancer disease state such as a group of peptide structures identified in Tables 43B, 43C, or 43D.

Step 704 may include reducing the plurality of peptide structure profiles using LASSO regression to identify a final group of peptide structures identified in Table 41 above. Step 704 may include reducing the plurality of peptide structure profiles using LASSO regression to identify a final group of peptide structures identified in Table 42 above. Step 704 may include reducing the plurality of peptide structure profiles using LASSO regression to identify a final group of peptide structures identified in Tables 43B, 43C, or 43D above.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the ovarian cancer disease state and a negative diagnosis for any subject of the plurality of subjects determined not to have the ovarian cancer disease state.

The machine learning model can include a binary classification model. Some binary classification models can include logistical regression models. Some logistical regression models can include LASSO regression models.

An alternative or additional step in process 700 can include filtering the initial plurality of peptide structure profiles by a coefficient of variation to generate a plurality of peptide structure profiles for use in training the machine learning model. As one example, only those peptide structure profiles having a low coefficient of variation (<20%) were included int the plurality of peptide structure profiles used for training.

An alternative or additional step in process 700 can include performing a differential expression analysis using initial training data to compare a first portion of the plurality of subjects diagnosed with the positive diagnosis for the ovarian cancer disease state versus a second portion of the plurality of subjects diagnosed with the negative diagnosis for the ovarian cancer disease state.

An alternative or additional step in process 700 can include identifying a first portion of the plurality of samples for subjects with benign pelvic tumors and malignant pelvic tumors and a second portion of the plurality of samples for subjects with a healthy status. An alternative or additional step in process 700 can include generating a training set of peptide structure profiles for 80% of the first portion and a test set of peptide structure profiles for a remaining 20% of the first portion and the second portion.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

### VI.K. Combined Training and Diagnosis

The exemplary methodologies described in Section VI. may be used to diagnose a subject who may have FLD or suspected of having from FLD. This diagnosis may be used to determine a method of treatment for a subject. The embodiments described herein may enable faster and more accurate diagnosis of the presence of NASH, including a stage of NASH. Being able to more quickly and accurately diagnose a subject (or patient) that has advanced from non-NASH to NASH in the FLD progression, or who has early stage NASH, may enable treating the subject more quickly, which may lead to a more desirable treatment outcome for the subject. Further, being able to more quickly and accurately determine when a subject has NASH and is at risk for advancement of the stage of NASH, including to late stage NASH, may be particularly useful in reducing the need for hospitalization and avoidance of death. In various embodiments, the severity of one or more symptoms of NASH may be reduced or reversed following use of methods of the disclosure.

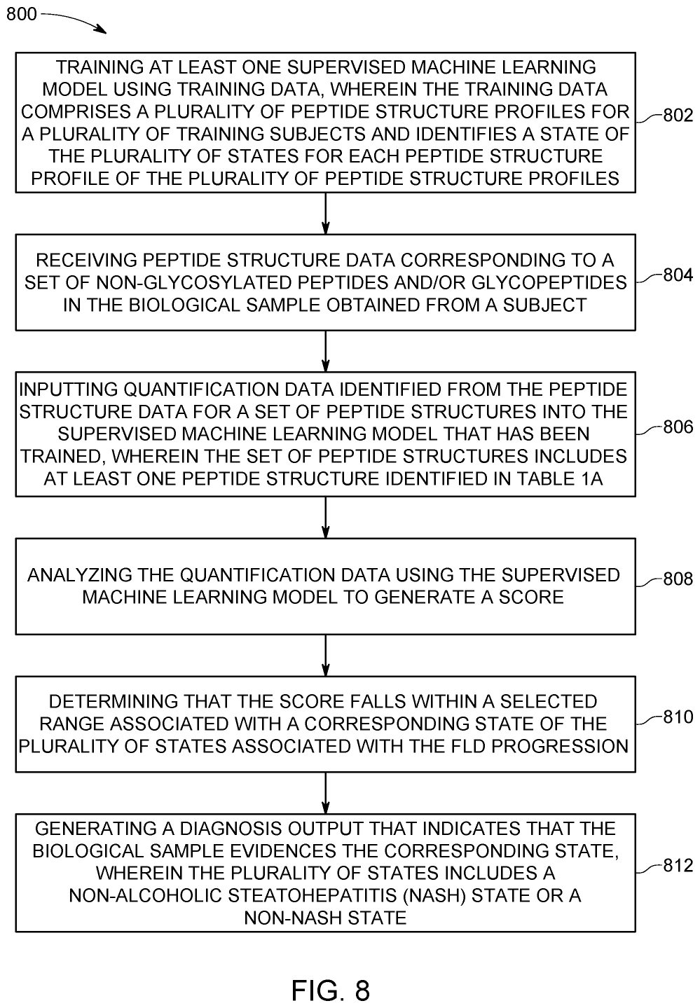

FIG. 8 is a flow chart of a process for classifying a biological sample as corresponding to one of a plurality of states associated with fatty liver disease (FLD) progression in accordance with various embodiments. Process 800 may be implemented using at least a portion of workflow 100 as described FIGS. 1, 2A, and/or 2B and/or analysis system 300 as described in FIG. 3.

Step 802 includes training a supervised machine learning model using training data. In various embodiments, the training data comprises a plurality of peptide structure profiles for a plurality of training subjects and identifies a corresponding state of the plurality of states for each peptide structure profile of the plurality of peptide structure profiles.

Step 804 includes receiving peptide structure data corresponding to a set of non-glycosylated peptides or glycopeptides in the biological sample obtained from a subject, such as any one or more from Table 1A.

Step 806 includes inputting quantification data identified from the peptide structure data for a set of peptide structures into the supervised machine learning model that has been trained. In some embodiments, the set of peptide structures includes at least one peptide structure identified in Table 1A or 1B.

Step 808 includes analyzing the quantification data using the supervised machine learning model to generate a score.

Step 810 includes determining that the score falls within a selected range associated with a corresponding state of the plurality of states associated with the FLD progression.

Step 812 includes generating a diagnosis output that indicates that the biological sample evidences the corresponding state. In some embodiments, the plurality of states includes a non-alcoholic steatohepatitis (NASH) state, a non-NASH (e.g., control or healthy) state, early stage NASH, or late stage NASH.

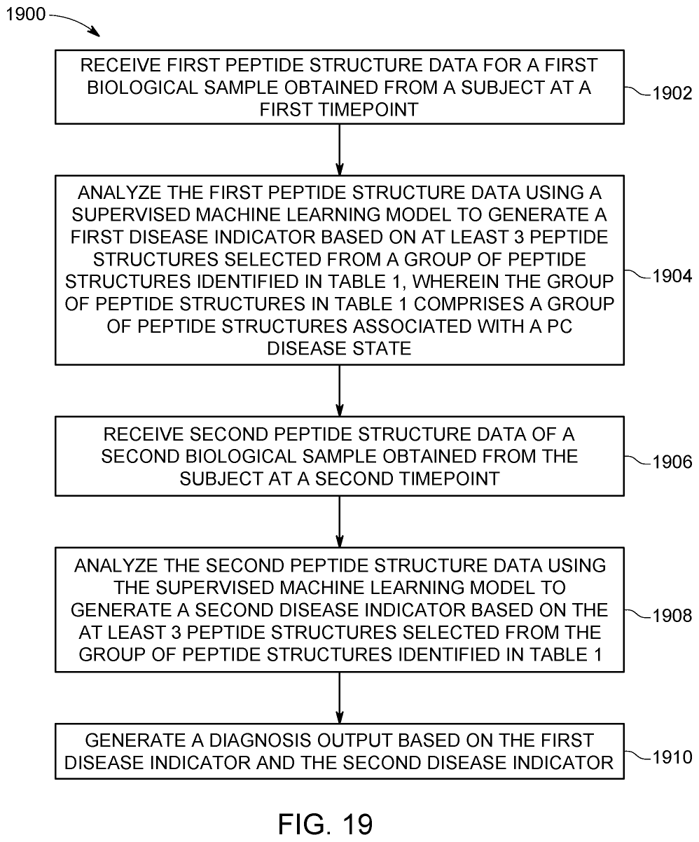

FIG. 19 is a flowchart of a process for monitoring a subject for a pancreatic cancer (PC) disease state in accordance with one or more embodiments. Process 1900 may be implemented using, for example, at least a portion of workflow 100 as described in FIGS. 1, 2A, and 2B and/or analysis system 300 as described in FIG. 3.

Step 1902 includes receiving first peptide structure data for a first biological sample obtained from a subject at a first timepoint.

Step 1904 includes analyzing the first peptide structure data using a supervised machine learning model to generate a first disease indicator based on at least 1 peptide structure selected from a group of peptide structures identified in Table 16. The group of peptide structures in Table 16 includes a group of peptide structures associated with a PC disease state in accordance with various embodiments. The supervised machine can be a binary classification model. In some embodiments, the binary classification model can be a logistical regression model.

Step 1906 includes receiving second peptide structure data of a second biological sample obtained from the subject at a second timepoint.

Step 1908 includes analyzing the second peptide structure data using the supervised machine learning model to generate a second disease indicator based on the at least 1 peptide structure selected from the group of peptide structures identified in Table 16.

Step 1910 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the PC disease state and the second biological sample evidences the positive diagnosis for the PC disease. In other embodiments, the diagnosis output identifies whether a non-PC disease state has progressed to the PC disease state, wherein the non-PC disease state includes either a healthy state, a control state, or a benign pancreatitis state.

### VI.L. Combined Training and Diagnosis for Diagnosis of NSCLC

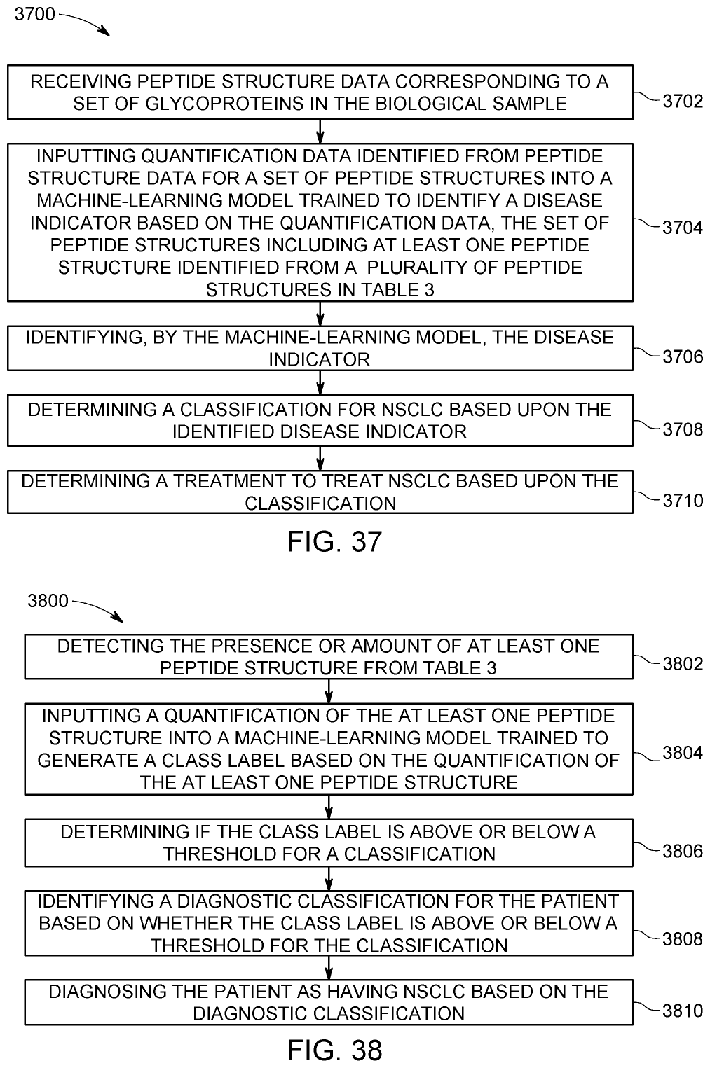

FIG. 37 illustrates a flow diagram 3700 of a method for treating NSCLC in a subject, in accordance with the presently disclosed embodiments. The flow diagram 3700 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 3700 may begin at block 3702 with one or more processing devices (e.g., computing platform 302) receiving peptide structure data corresponding to a set of glycoproteins in the biological sample. The flow diagram 3700 may then continue at block 3704 with one or more processing devices (e.g., computing platform 302) inputting quantification data identified from the peptide structure data for a set of the peptide structures into a machine-learning model trained to identify a disease indicator based on the quantification data, wherein the set of peptide structure data comprises at least one peptide structure identified from a plurality of peptide structures in Table 35 and/or Table 40. The flow diagram 3700 may then continue at block 3706 with one or more processing devices (e.g., computing platform 302) identifying, by the machine-learning model, the disease indicator. The flow diagram 3700 may then continue at block 3708 with one or more processing devices (e.g., computing platform 302) determining a classification for NSCLC based upon the identified disease indicator. The flow diagram 3700 may then conclude at block 3710 with one or more processing devices (e.g., computing platform 302) determining a treatment to treat NSCLC based upon the classification.

FIG. 38 illustrates a flow diagram 3800 of a method for diagnosing an individual with NSCLC, in accordance with the presently disclosed embodiments. The flow diagram 3800 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 3800 may begin at block 3802 with one or more processing devices (e.g., computing platform 302) detecting the presence or amount of at least one structure structures from Table 35 and/or Table 40. The flow diagram 3800 may begin at block 3802 with one or more processing devices (e.g., computing platform 302) detecting the presence or amount of at least one peptide structure structures from Table 40. The flow diagram 3800 may then continue at block 3804 with one or more processing devices (e.g., computing platform 302) inputting a quantification of the detected at least one peptide structure into a machine-learning model trained to generate a class label. The flow diagram 3800 may then continue at block 3806 with one or more processing devices (e.g., computing platform 302) determining if the class label is above or below a threshold for a classification. The flow diagram 3800 may then continue at block 3808 with one or more processing devices (e.g., computing platform 302) identifying a diagnostic classification for a patient based on whether the class label is above or below a threshold for the classification. The flow diagram 3800 may then conclude at block 3810 with one or more processing devices (e.g., computing platform 302) diagnosing the patient as having NSCLC based on the diagnostic classification.

### VII. Exemplary Methodologies for Treatment

### VII.A. Treating NASH

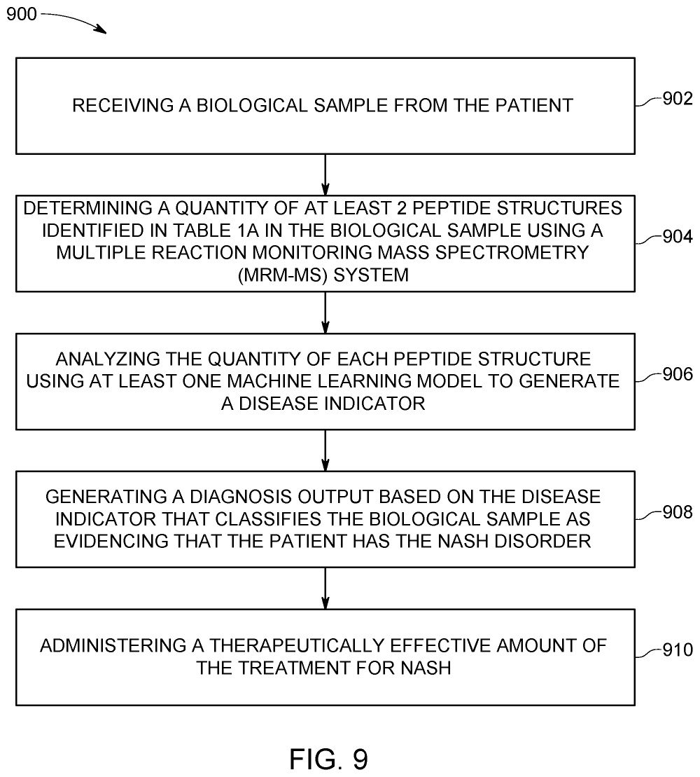

FIG. 9 is a flowchart of a process for treating a subject for NASH in accordance with various embodiments. Process 900 may be at least partially implemented using at least a portion of workflow 100 as described FIGS. 1, 2A, and/or 2B and/or analysis system 300 as described in FIG. 3.

Step 902 includes receiving a biological sample. The biological sample may be one that is obtained from a patient, and in specific cases the sample comprises serum.

Step 904 includes determining a quantity of each peptide structure identified in a predetermined list using an MRM-MS system. The predetermined list may be, for example, the list identified in Table 1A or 1B, or a subset thereof.

Step 906 includes analyzing the quantity of each peptide structure using a machine learning model to generate a disease indicator.

Step 908 includes generating a diagnosis output based on the disease indicator that classifies the biological sample as evidencing that the patient has NASH (or the NASH disorder).

Step 910 includes administering a treatment for NASH to the patient, such as via at least one of intravenous or oral administration of the treatment at therapeutic dosage(s). The treatment may be comprised of one or more therapeutics or derivatives thereof.

The treatment may include, for example, without limitation, at least one compound or derivative thereof selected from the group consisting of Obeticholic acid (OCA), Tropifexor, Elafibranor, Saroglitazar, Aramchol, Semaglutide, Tirzepatide, Cotadutide, NGM282, MSDC-0602K, Resmetirom, Cenicriviroc, Selonsertib, Emricasan, Simtuzumab, GR-MD-02, and a combination thereof. In various embodiments, as an example a therapeutic dosage for Obeticholic acid (OCA) may include a dosage within a range of 10-25 mg daily.

Process 900 may include one or more additional steps. For example, process 900 may further include designing the therapeutic for treating the subject in response to determining that the biological sample obtained from the subject evidences NASH. Process 900 may include generating a treatment plan for treating the subject in response to determining that the biological sample obtained from the subject evidences NASH.

### VII.B. Treating NSCLC

In some embodiments, provided herein are methods of treating NSCLC based upon the presence, amount, and/or relative amount of one or more biomarkers provided herein. In some embodiments, the method comprises treating NSCLC based upon the presence, amount, and/or relative amount of one or more peptide structure from Table 35. In some embodiments, the method comprises treating NSCLC based upon the presence, amount, and/or relative amount of one or more peptide structures from Table 40. In some embodiments, the method comprises classifying a biological sample with respect to a plurality of states associated with NSCLC based upon one or more peptide structure provided herein and selecting a treatment for NSCLC based upon the classification. In some embodiments, the method comprises classifying a biological sample with respect to a plurality of states associated with NSCLC based upon one or more peptide structure provided in Table 35 and/or Table 40 and administering a treatment for NSCLC based upon the classification. In some embodiments, the method comprises classifying a biological sample with respect to a plurality of states associated with NSCLC based upon one or more peptide structure provided herein and administering a treatment for NSCLC based upon the classification. In some embodiments, the method comprises classifying a biological sample with respect to a plurality of states associated with NSCLC based upon one or more peptide structure provided in Table 35 and/or Table 40 and administering a treatment for NSCLC based upon the classification. In some embodiments, the method comprises inputting quantification data identified from peptide structure data for a set of peptides to identify a disease indicator, detecting the presence of a corresponding state associated with NSCLC in response that the disease indicator falls within a selected range, and diagnosing NSCLC. In some embodiments, the peptide structure data comprises one or more peptide structure provided in Table 35. In some embodiments, the peptide structure data comprises one or more peptide structure provided in Table 40. In some embodiments, the method further comprises administering an effective amount of a therapy for NSCLC. In some embodiments, the method further comprises selecting a particular therapy based upon the disease indicator.

In some embodiments, provided herein is a method of treating NSCLC comprising detecting the presence and/or amount of at least one peptide structure from Table 35 and selecting a NSCLC therapy. In some embodiments, method of treating NSCLC further comprises administering an effective amount of a NSCLC therapy to the individual based upon the presence and/or amount of at least one peptide structure from Table 35. In some embodiments, the diagnosis and/or treatment is based upon the presence and/or amount of at least two, at least three, at least four, at least five, at least 10, at least 15, at least 20, or at least 25 peptide structures from Table 35. In some embodiments, the method of treating NSCLC comprises detecting the presence (or absence) or amount of at least one peptide structure from Table 40 and selecting a NSCLC therapy. In some embodiments, the method of treating NSCLC comprises detecting the presence (or absence) or amount of at least one peptide structure from Table 40 and administering an effective amount of a NSCLC therapy to the individual. In some embodiments, the method further comprises selecting a therapy based upon the presence, and/or amount of the at least one peptide structure from Table 40. In some embodiments, the diagnosis and/or treatment is based upon the presence and/or amount of at least two, at least three, at least four, at least five, at least 10, or at least 15 peptide structures from Table 40.

In some embodiments, the method comprises selecting a therapy to treat NSCLC. In some embodiments, the method of selecting a therapy for NSCLC comprises inputting quantification data identified from peptide structure data for a set of peptides and/or glycopeptides into one or more machine-learning model trained to identify a disease indicator. In some embodiments, the method of selecting a therapy comprises classifying the sample as having NSCLC or not having NSCLC based upon the disease indicator. In some embodiments, the therapy is selected based upon presence and/or amount of at least one, at least two, at least three, at least four, at least five, at least ten, or at least 15 peptide structures from Table 35 and/or Table 40. In some embodiments, the therapy is selected based upon presence and/or amount of at least one, at least two, at least three, at least four, at least five, at least ten, or at least 15 peptides and/or glycopeptides comprising the amino acid sequence of SEQ ID NOs: 224-296 along with the associated glycan set forth in Table 35. In some embodiments, the presence, absence, and/or amount of one or more peptides and/or glycopeptide is determined by MRM-MS. In some embodiments, the therapy is selected on the basis of the stage of NSCLC. In some embodiments, the therapy is selected on the basis of one or more NSCLC risk factor in combination with the presence, absence, and or amount of one or more peptides or glycopeptides provided herein. In some embodiments, the therapy for NSCLC is selected from the group comprising a surgery, a chemotherapeutic therapy, a patient-specific therapy, a targeted immunotherapy, a radiation procedure, a radiofrequency ablation (RFA) procedure, or a combination thereof. In some embodiments, the surgery comprises the removal of one or more parts of the lung. In some embodiments, the platinum-coordinating compound comprises one of cisplatin, carboplatin, and nedaplatin. In some embodiments, the chemotherapeutic comprises docetaxel, paclitaxel, albumin-bound paclitaxel, vinorelbine, gemcitabine, irinotecan, pemetrexed, tegafur/gimeracil/oteracil, etoposide, or a combination thereof. In some embodiments, the chemotherapeutic therapy is a platinum-doublet regimen. In some embodiments, the chemotherapeutic therapy is a platinum-triple regimen. In some embodiments, the targeted immunotherapy comprises one or more antibody directed towards an immune system checkpoint protein including but not limited to PD-1, PD-L1, and CTLA-4. In some embodiments, the therapy for NSCLC comprises a combination of one or more antibody that targets PD-1, PD-L1, and CTLA-4. In some embodiments, the targeted therapy comprises one or more patient-specific therapy agent selected based on patient-specific changes in tumor cell gene expression including but not limited to changes in KRAS, EGFR, ALK, ROS1, BRAF, RET, MET, and NTRK genes. In some embodiments, the patient-specific therapy is an inhibitor of an oncogene. In some embodiments, the patient-specific therapy is an inhibitor of one or more of KRAS, EGFR, ALK, ROS1, BRAF, MEK, RET, MET, and NTRK. In some embodiments, the radiation procedure comprises the use of high-energy rays or particles to treat NSCLC. In some embodiments, the brachytherapy comprises the placement of radioactive material in or adjacent to the tumor in the airway (e.g., bronchial tubes).

In some embodiments, the method comprises administering a therapy to treat NSCLC. In some embodiments, the method of administering a therapy comprises inputting quantification data identified from peptide structure data for a set of peptides and/or glycopeptides into one or more machine-learning model trained to identify a disease indicator. In some embodiments, the method of administering a therapy comprises classifying the sample as having NSCLC or not having NSCLC based upon the disease indicator. In some embodiments, the therapy is administered based upon presence and/or amount of at least one, at least two, at least three, at least four, at least five, at least ten, or at least 15 peptide structures from Table 35 and/or Table 40. In some embodiments, the therapy is administered based upon presence and/or amount of at least one, at least two, at least three, at least four, at least five, at least ten, or at least 15 peptides and/or glycopeptides comprising the amino acid sequence of SEQ ID NOs: 224-296 along with the associated glycan set forth in Table 35. In some embodiments, the presence, absence, and/or amount of one or more peptides and/or glycopeptide is determined by MRM-MS. In some embodiments, the therapy is administered on the basis of the stage of NSCLC. In some embodiments, the therapy is administered on the basis of one or more NSCLC risk factor in combination with the presence, absence, and or amount of one or more peptides or glycopeptides provided herein. In some embodiments, the therapy administered for NSCLC is selected from the group comprising a surgery, a chemotherapeutic therapy, a patient-specific therapy, a targeted immunotherapy, a radiation procedure, a radiofrequency ablation (RFA) procedure, or a combination thereof. In some embodiments, the surgery comprises the removal of one or more parts of the lung. In some embodiments, the platinum-coordinating compound comprises one of cisplatin, carboplatin, and nedaplatin. In some embodiments, the chemotherapeutic comprises docetaxel, paclitaxel, albumin-bound paclitaxel, vinorelbine, gemcitabine, irinotecan, pemetrexed, tegafur/gimeracil/oteracil, etoposide, or a combination thereof. In some embodiments, the chemotherapeutic therapy is a platinum-doublet regimen. In some embodiments, the chemotherapeutic therapy is a platinum-triple regimen. In some embodiments, the targeted immunotherapy comprises one or more antibody directed towards an immune system checkpoint protein including but not limited to PD-1, PD-L1, and CTLA-4. In some embodiments, the therapy for NSCLC comprises a combination of one or more antibody that targets PD-1, PD-L1, and CTLA-4. In some embodiments, the patient-specific therapy comprises one or more patient-specific therapy agent selected based on patient-specific changes in tumor cell gene expression including but not limited to changes in KRAS, EGFR, ALK, ROS1, BRAF, RET, MET, and NTRK genes. In some embodiments, the patient-specific therapy is an inhibitor of an oncogene. In some embodiments, the patient-specific therapy is an inhibitor of one or more of KRAS, EGFR, ALK, ROS1, BRAF, MEK, RET, MET, and NTRK. In some embodiments, the radiation procedure comprises the use of high-energy rays or particles to treat NSCLC. In some embodiments, the brachytherapy comprises the placement of radioactive material in or adjacent to the tumor in the airway (e.g., bronchial tubes).

In some embodiments, the method comprises administering a therapy to treat NSCLC. In some embodiments, the method of administering a therapy comprises inputting quantification data identified from peptide structure data for a set of peptides into one or more machine-learning model trained to identify a disease indicator. In some embodiments, the set of peptide structures comprising one or more, two or more, three or more, four or more, five or more, 10 or more, 15 or more, or each of the peptides and/or glycopeptides consisting of the amino acid sequence of SEQ ID NOs: 224-257. In some embodiments, the method comprises inputting quantification data from peptide structure data into a machine-learning model, wherein the machine-learning model comprises a model trained using one or more of a first set of peptide structure coefficients set forth in Table 39 for all stages of NSCLC (stages 1-4), a second set of peptide structure coefficients set forth in Table 39 for early-stage NSCLC (stages 1-2), and a third set of peptide structure coefficients set forth in Table 39 for late-stage NSCLC (stages 3-4). In some embodiments, the model is trained using any one of the set of peptide structure coefficients described herein to identify a disease indicator, identifying by the machine-learning model, the disease indicator, and classifying the biological sample with respect to a plurality of states associated with NSCLC based upon the disease indicator. In some embodiments, the disease indicator comprises one or more scores that indicate a probability that the subject falls within one of the states associated with NSCLC (e.g., having NSCLC or not having NSCLC). In some embodiments, the model trained using any one of the first set, the second set, or the third set of peptide structure coefficients is a logical regression model. In some embodiments, the model trained using any one of the first set, the second set, or the third set of peptide structure coefficients comprises a LASSO regression model. In some embodiments, the peptide structures are detected using LC-MS. In some embodiments, the LC-MS comprises LC-MS/MS and LC-MS/MS running in a MRM mode. In some embodiments, the method comprises selecting a treatment for NSCLC based upon the classification described herein. In some embodiments, the method comprises administering a treatment for NSCLC based upon the classification described herein. In some embodiments, the therapy for NSCLC is selected from the group comprising a surgery, a chemotherapeutic therapy, a patient-specific therapy, a targeted immunotherapy, a radiation procedure, a radiofrequency ablation (RFA) procedure, or a combination thereof. In some embodiments, the surgery comprises the removal of one or more parts of the lung. In some embodiments, the therapy comprises a lobectomy, a bronchial sleeve resection, a wedge resection, or a pneumonectomy. In some embodiments, the method comprises classifying a biological sample with respect to a plurality of states associated with NSCLC based upon one or more peptide structures provided in Table 35 or Table 40. In some embodiments, the method comprises inputting quantification data identified from peptide structure data for a set of peptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptides is determined by MRM-MS. In some embodiments, the method comprises selecting a particular therapy described herein based upon the presence, amount, and/or relative amount of one or more biomarkers comprising the peptide structures provided in Table 35 or Table 40. In some embodiments, the method comprises administering a particular therapy described herein based upon the presence, amount, and/or relative amount of one or more biomarkers comprising the peptide structures provided in Table 35 or Table 40. In some embodiments, the method further comprises selecting a particular therapy described herein based upon the disease indicator and/or classification. In some embodiments, the method further comprises administering a particular therapy described herein based upon the disease indicator and/or classification.

In some embodiments, the chemotherapeutic therapy comprises a platinum-coordinating compound, a chemotherapeutic, or a combination thereof. In some embodiments, the platinum-coordinating compound comprises one of cisplatin (CDDP), carboplatin (CBDCA), and nedaplatin (CDGP). In some embodiments, the chemotherapeutic comprises docetaxel (Taxotere, DTX), paclitaxel (Taxol, PTX), albumin-bound paclitaxel (nab-paclitaxel, Abraxane), vinorelbine (Navelbine,VNR), gemcitabine (Gemzar, GEM), irinotecan (CPT-11), pemetrexed (Alimta, PEM), tegafur/gimeracil/oteracil (S1), etoposide (VP-16), or a combination thereof. In some embodiments, the chemotherapeutic therapy is a platinum-doublet regimen. In some embodiments, the chemotherapeutic therapy is a platinum-triple regimen. In some embodiments, the method comprises classifying a biological sample with respect to a plurality of states associated with NSCLC based upon one or more peptide structures provided in Table 35 or Table 40. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptides is determined by MRM-MS. In some embodiments, the method comprises selecting a particular therapy described herein based upon the presence, amount, and/or relative amount of one or more biomarkers comprising the peptide structures provided in Table 35 or Table 40. In some embodiments, the method comprises selecting a particular therapy described herein based upon the presence, amount, and/or relative amount of one or more biomarkers comprising the glycopeptides provided in Table 35 or Table 40. In some embodiments, the method comprises administering a particular therapy described herein based upon the presence, amount, and/or relative amount of one or more biomarkers comprising the peptide structures provided in Table 35 or Table 40. In some embodiments, the method comprises administering a particular therapy described herein based upon the presence, amount, and/or relative amount of one or more biomarkers comprising the glycopeptides provided in Table 35 or Table 40. In some embodiments, the method further comprises selecting a particular therapy described herein based upon the disease indicator and/or classification. In some embodiments, the method further comprises administering a particular therapy described herein based upon the disease indicator and/or classification.

In some embodiments, the targeted immunotherapy comprises one or more antibody directed towards an immune system checkpoint protein including but not limited to PD-1, PD-L1, and CTLA-4. In some embodiments, the antibody targeting PD-1 comprises nivolumab (Opdivo), pembrolizumab (Keytruda), and cemiplimab (Libtayo). In some embodiments, the antibody targeting PD-L1 comprises atezolizumab (Tecentriq), durvalumab (Imfinzi), and avelumab (Bavencio). In some embodiments, the antibody targeting CTLA-4 comprises ipilimumab (Yervoy). In some embodiments, the therapy for NSCLC comprises a combination of one or more antibody that targets PD-1, PD-L1, and CTLA-4. In some embodiments, the method comprises classifying a biological sample with respect to a plurality of states associated with NSCLC based upon one or more peptide structures provided in Table 35 or Table 40. In some embodiments, the method comprises classifying a biological sample with respect to a plurality of states associated with NSCLC based upon one or more glycopeptides provided in Table 35 or Table 40. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 35 or Table 40. In some embodiments, the method comprises selecting a particular therapy described herein based upon the presence, amount, and/or relative amount of one or more biomarkers comprising the glycopeptides provided in Table 35 or Table 40. In some embodiments, the method comprises administering a particular therapy described herein based upon the presence, amount, and/or relative amount of one or more biomarkers comprising the peptide structures provided in Table 35 or Table 40. In some embodiments, the method comprises administering a particular therapy described herein based upon the presence, amount, and/or relative amount of one or more biomarkers comprising the glycopeptides provided in Table 35 or Table 40. In some embodiments, the method further comprises selecting a particular therapy described herein based upon the disease indicator and/or classification. In some embodiments, the method further comprises administering a particular therapy described herein based upon the disease indicator and/or classification.

In some embodiments, the therapy comprises one or more patient-specific therapy agent selected based on patient-specific changes in tumor cell gene expression including but not limited to changes in KRAS, EGFR, ALK, ROS1, BRAF, RET, MET, and NTRK genes. In some embodiments, the patient-specific therapy is an inhibitor of an oncogene. In some embodiments, the patient-specific therapy is an inhibitor of one or more of KRAS, EGFR, ALK, ROS1, BRAF, MEK, RET, MET, and NTRK. In some embodiments, the patient-specific therapy comprises one or more of sotorasib (Lumakras), erlotinib (Tarceva), afatinib (Gilotrif), gefitinib (Iressa), osimertinib (Tagrisso), dacomitinib (Vizimpro), amivantamab (Rybrevant), nobocertinib (Exkivity), necitumumab (Portrazza), crizotinib (Xalkori), ceritinib (Zykadia), alectinib (Alecensa), brigatinib (Alunbrig), lorlatinib (Lorbrena), entrectinib (Rozlytrek), dabrafenib (Tafinlar), trametinib (Mekinist), selpercatinib (Retevmo), pralsetinib (Gavreto), capmatinib (Tabrecta), tepotinib (Tepmetko), larotrectinib (Vitrakvi), and combinations thereof. In some embodiments the patient-specific therapy comprises an angiogenesis inhibitor. In some embodiments, the angiogenesis inhibitor comprises one of bevacizumab (Avastin, BEV) and ramucirumab (Cyramza, RAM). In some embodiments, the therapy for NSCLC comprises a combination of one or more patient-specific therapy agents. In some embodiments, the method comprises classifying a biological sample with respect to a plurality of states associated with NSCLC based upon one or more peptide structures provided in Table 35 or Table 40. In some embodiments, the method comprises classifying a biological sample with respect to a plurality of states associated with NSCLC based upon one or more glycopeptides provided in Table 35 or Table 40. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 35 or Table 40. In some embodiments, the method comprises selecting a particular therapy described herein based upon the presence, amount, and/or relative amount of one or more biomarkers comprising the glycopeptides provided in Table 35 or Table 40. In some embodiments, the method comprises administering a particular therapy described herein based upon the presence, amount, and/or relative amount of one or more biomarkers comprising the peptide structures provided in Table 35 or Table 40. In some embodiments, the method comprises administering a particular therapy described herein based upon the presence, amount, and/or relative amount of one or more biomarkers comprising the glycopeptides provided in Table 35 or Table 40. In some embodiments, the method further comprises selecting a particular therapy described herein based upon the disease indicator and/or classification. In some embodiments, the method further comprises administering a particular therapy described herein based upon the disease indicator and/or classification.

In some embodiments, the radiation procedure comprises the use of high-energy rays or particles to treat NSCLC. In some embodiments, the radiation procedure comprises external beam radiation therapy (EBRT) and internal radiation therapy (also referred to as brachytherapy). In some embodiments, the EBRT comprises one or more of stereotactic ablative radiotherapy (SABR), three-dimensional conformal radiation therapy (3D-CRT), intensity modulated radiation therapy (IMRT), volumetric modulated arc therapy (VMAT), and stereotactic radiosurgery (SRS). In some embodiments, the brachytherapy comprises the placement of radioactive material in or adjacent to the tumor in the airway (e.g., bronchial tubes). In some embodiments, the method comprises classifying a biological sample with respect to a plurality of states associated with NSCLC based upon one or more peptide structures provided in Table 35 or Table 40. In some embodiments, the method comprises classifying a biological sample with respect to a plurality of states associated with NSCLC based upon one or more glycopeptides provided in Table 35 or Table 40. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 35 or Table 40. In some embodiments, the method comprises selecting a particular therapy described herein based upon the presence, amount, and/or relative amount of one or more biomarkers comprising the glycopeptides provided in Table 35 or Table 40. In some embodiments, the method comprises administering a particular therapy described herein based upon the presence, amount, and/or relative amount of one or more biomarkers comprising the peptide structures provided in Table 35 or Table 40. In some embodiments, the method comprises administering a particular therapy described herein based upon the presence, amount, and/or relative amount of one or more biomarkers comprising the glycopeptides provided in Table 35 or Table 40. In some embodiments, the method further comprises selecting a particular therapy described herein based upon the disease indicator and/or classification. In some embodiments, the method further comprises administering a particular therapy described herein based upon the disease indicator and/or classification.

In some embodiments, the method involves monitoring of the individual for progression of NSCLC. In some embodiments, the method involving monitoring comprises inputting quantification data identified from peptide structure data for a set of peptides and/or glycopeptides into a machine-learning model trained to identify a disease indicator. In some embodiments, the machine-learning model is a regularized regression model (e.g., LASSO regression model) trained using one or more of a first set, a second set, or a third set of peptide structure coefficients set forth in Table 39. In some embodiments, the first set, the second set, or the third set of peptide structure coefficients used to train the model comprise one or more of the amino acid sequence of SEQ ID NOs: 224-257 as set forth in Table 39. In some embodiments, the method comprises classifying the sample as having NSCLC or not having NSCLC based upon the disease indicator. In some embodiments, the method involving monitoring comprises classifying a subsequent biological sample with respect to a plurality of states associated with NSCLC, based upon one or more peptide structures provided in Table 35 and/or Table 40, and selecting a treatment for NSCLC based upon the classification. In some embodiments, the monitoring comprises classifying a subsequent biological sample with respect to a plurality of states associated with NSCLC based upon one or more peptide structures provided in Table 35 and/or Table 40 and administering a treatment for NSCLC based upon the classification. In some embodiments, the presence, absence, and/or amount of one or more peptides and/or glycopeptide is determined by MRM-MS. In some embodiments, the method involving monitoring comprises inputting quantification data identified from peptide structure data for a set of peptides and/or glycopeptides to identify a disease indicator, detecting the presence of a corresponding state associated with NSCLC in response that the disease indicator falls within a selected range, and diagnosing NSCLC. In some embodiments, the peptide structure data comprises one or more peptide structure provided in Table 35 and/or Table 40. In some embodiments, the peptide structure data comprises one or more glycopeptide structure provided in Table 35 and/or Table 40. In some embodiments, the method involving monitoring further comprises selecting a particular therapy based upon the disease indicator. In some embodiments, the method involving monitoring further comprises administering an effective amount of a therapy for NSCLC.

### VII.C. Patient Populations—NSCLC

Provided herein is a method of diagnosis and treatment for an individual. Further provided herein is a method of diagnosis and treatment for an individual with one or more risk factors associated with NSCLC. In some embodiments, the method comprises measuring the amount/presence or absence of one or more peptides structures from Table 35 in an individual with one or more risk factors associated with NSCLC. In some embodiments, the method involves diagnosing an individual based upon presence and/or amount of one or more peptide structures from Table 35. In some embodiments, the method involves diagnosing an individual based upon presence and/or amount of one or more glycopeptides from Table 35. In some embodiments, the diagnosis is based upon the presence and/or amount of one or more peptides and/or glycopeptides comprising the amino acid sequence of SEQ ID NOs: 224-296 set forth in Table 35. In some embodiments, the diagnosis is based upon the presence and/or amount of one or more glycopeptides comprising the amino acid sequence of SEQ ID NOs: 224-296 set forth in Table 35. In some embodiments, the diagnosis is based upon the presence and/or amount of one or more glycopeptides consisting of the amino acid sequence of SEQ ID NOs: 224-296 along with the associated glycan set forth in Table 35. In some embodiments, the individual diagnosed with NSCLC is administered one or more NSCLC therapies described herein, based on the disease indicator determined by the diagnosis. In some embodiments, the individual is administered one or more NSCLC therapies described herein, based on the disease indicator determined by the diagnosis. In some embodiments, the individual confirmed to have NSCLC is treated based on the disease indicator determined by the diagnosis.

In some embodiments, the individual is diagnosed, wherein one or more peptide structures from Table 35 are detected and are distinct from a healthy control sample. In some embodiments, the individual is diagnosed, wherein one or more peptide structures comprising the amino acid sequence of SEQ ID NOs: 224-296 are detected and are distinct from a healthy control sample. In some embodiments, the individual is diagnosed, wherein one or more glycopeptides comprising the amino acid sequence of SEQ ID NOs: 224-296 are detected and are distinct from a healthy control sample. In some embodiments, the amount of at least one peptide structure is none, or below a detection limit. In some embodiments, the amount of at least one glycopeptide structure is none, or below a detection limit. In some embodiments, the amount of at least one peptide structure from Table 35 is none, or below a detection limit. In some embodiments, the amount of at least one peptide structure comprising the amino acid sequence of SEQ ID NOs: 224-296 set forth in Table 35 is none, or below a detection limit. In some embodiments, the amount of at least one peptide structure is significantly lower than a control sample from a healthy individual. In some embodiments, the amount of at least one glycopeptide structure is significantly lower than a control sample from a healthy individual. In some embodiments, the amount of at least one peptide structure from Table 35 is significantly lower than a control sample from a healthy individual. In some embodiments, the amount of at least one peptide structure comprising the amino acid sequence of SEQ ID NOs: 224-296 set forth in Table 35 is significantly lower than a control sample from a healthy individual. In some embodiments, the amount of at least one peptide structure is significantly higher than a control sample from a healthy individual. In some embodiments, the amount of at least one glycopeptide structure is significantly higher than a control sample from a healthy individual. In some embodiments, the amount of at least one peptide structure from Table 35 is significantly higher than a control sample from a healthy individual. In some embodiments, the amount of at least one peptide structure comprising the amino acid sequence of SEQ ID NOs: 224-296 set forth in Table 35 is significantly higher than a control sample from a healthy individual. In some embodiments, the individual is diagnosed and treated according to the presence and/or amount of one or more peptide structures from Table 35. In some embodiments, the individual is diagnosed and treated according to the presence and/or amount of one or more peptide structures comprising the amino acid sequence of SEQ ID NOs: 224-296 along with the associated glycan set forth in Table 35.

In some embodiments, the individual has NSCLC. In some embodiments, the individual has stage 0, stage I, stage II, stage III, or stage IV NSCLC. In some embodiments, the individual has early-stage NSCLC. In some embodiments, the individual has late-stage NSCLC or advanced NSCLC. In some embodiments, the individual has NSCLC that has not spread from the site of origination. In some embodiments, the individual has NSCLC that has spread locally to the surrounding tissue. In some embodiments, the individual has NSCLC that has spread beyond the original tumor and/or the local tumor environment. In some embodiments, the individual has NSCLC that has spread to one or more organs beyond the lungs. In some embodiments, the individual has metastatic NSCLC. In some embodiments, the individual has NSCLC and has relapsed and/or progressed. In some embodiments, the method comprises classifying a biological sample with respect to a plurality of states associated with NSCLC based upon one or more peptide structures provided in Table 35 or Table 40. In some embodiments, the method comprises classifying a biological sample with respect to a plurality of states associated with NSCLC based upon one or more glycopeptides provided in Table 35 or Table 40. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 35 and/or Table 40. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 35 or Table 40. In some embodiments, the method comprises selecting a particular therapy described herein based upon the presence, amount, and/or relative amount of one or more biomarkers comprising the glycopeptides provided in Table 35 or Table 40. In some embodiments, the method comprises administering a particular therapy described herein based upon the presence, amount, and/or relative amount of one or more biomarkers comprising the peptide structures provided in Table 35 or Table 40. In some embodiments, the method comprises administering a particular therapy described herein based upon the presence, amount, and/or relative amount of one or more biomarkers comprising the glycopeptides provided in Table 35 or Table 40. In some embodiments, the method further comprises selecting a particular therapy described herein based upon the disease indicator and/or classification. In some embodiments, the method further comprises administering a particular therapy described herein based upon the disease indicator and/or classification.

In some embodiments, the individual has had prior lines of therapy for treating NSCLC. In some embodiments, the individual has had at least 1, at least 2, or at least 3 prior lines of therapy for treating NSCLC. In some embodiments, the individual has had no more than 1, no more than 2, or no more than 3 prior lines of therapy for treating NSCLC. In some embodiments, the individual has not had prior therapy for treating NSCLC.

In some embodiments, the individual has altered gene expression relevant for NSCLC treatment. In some embodiments, the individual has altered oncogene expression. In some embodiments, the individual has altered tumor cell gene expression. In some embodiments, the altered gene expression comprises altered gene expression of one or more of KRAS, EGFR, ALK, ROS1, BRAF, RET, MET, and NTRK. In some embodiments, the altered gene expression comprises altered gene expression of one or more immune system checkpoint proteins PD-1, PD-L1, and CTLA-4. In some embodiments, the individual having altered gene expression relevant for NSCLC treatment may benefit from a therapy comprising one or more antibody that targets PD-1, PD-L1, and CTLA-4, or a combination thereof.

In some embodiments, the individual is at risk of developing NSCLC. In some embodiments, the individual is positive for one or more risk factor that increases the chances of developing NSCLC. In some embodiments, the one or more risk factor is smoking, wherein the individual smokes at least one of cigarettes, cigars, pipes, and other tobacco-based products. In some embodiments, the individual is a smoker. In some embodiments, the individual smoked in the past and has quit smoking. In some embodiments, the individual is or has been a recreational smoker, wherein smoking occurs infrequently. In some embodiments, the individual is a non-smoker and has never smoked before. In some embodiments, the individual is exposed to secondhand smoke. In some embodiments, the individual is exposed to environmental or occupational carcinogens that increase the risk of developing NSCLC. In some embodiments, the environmental or occupational carcinogen comprises one or more of asbestos, arsenic, chromium, beryllium, nickel, soot, tar, indoor air pollution, and outdoor air pollution. In some embodiments, the individual is exposed to radiation that increase the risk of developing NSCLC. In some embodiments, the radiation comprises one or more of radiation therapy for the breast or chest, extensive CT scan imaging, and radon exposure. In some embodiments, the individual has a family history of lung cancer that increase the risk of developing NSCLC. In some embodiments, the individual is aging, wherein the individual is at least 40 years old. In some embodiments, the individual has at least 1, at least 2, at least 3, at least 4, at least 5, or at least 6 risk factors for NSCLC.

In some embodiments, the individual is at least 18 years old. In some embodiments, the individual is at least 20, at least 25, at least 30, at least 35, at least 40, at least 45, at least 50, at least 55, at least 60, at least 65, at least 70, at least 75, at least 80, at least 85, or at least 90 years old. In some embodiments, the individual is at least 65 years old.

In some embodiments, the individual is positive for one or more disease features of NSCLC. In some embodiments, the one or more disease features of NSCLC comprise a new cough, a worsening cough, a persistent cough, a cough that produces blood (e.g., hemoptysis), shortness of breath, a persistent chest infection (e.g., bronchitis, pneumonia), wheezing, chest pain, voice hoarseness, a headache, facial swelling, body swelling, upper body pain, weakness of the hand, a droopy eyelid, blurred vision, unexplained weight loss, tiredness, fatigue, persistent or worsening bone pain, and persistent or worsening joint pain. In some embodiments, the individual has at least 1, at least 2, at least 3, at least 4, at least 5, or at least 6 disease features of NSCLC. In some embodiments, the individual has any combination of disease features of NSCLC described herein.

### VII.D. Methods of Treating Ovarian Cancer

In one or more embodiments, the final output generated in step 506 in FIG. 47 or in step 606 in FIG. 48A may include a treatment output. The treatment output may identify one or more treatment types for a subject based on the disease indicator and/or diagnosis output generated via process 500 in FIG. 47 or process 600 in FIG. 48A, respectively. Treatment for ovarian cancer (e.g., EOC) may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment output may include, for example, a treatment plan. The treatment plan may include, for example, but is not limited to, a timeline or schedule for administering the treatment, dosing information, other treatment-related information, or a combination thereof. Being able to accurately predict malignancy via the process 500 in FIG. 47 and/or the process 600 in FIG. 48A may allow treatment for malignant pelvic tumors (e.g., EOC) to be started earlier without requiring, in many or most cases, further invasive testing such as a biopsy.

In one or more embodiments, a patient biological sample is obtained from a subject. The biological sample may be processed (e.g., via digestion and fragmentation) such that one or more peptide structures of interest are detected. For example, detection and quantification may be performed for one or more peptide structures from Table 41, Table 42, Table 43A, Table 43B, Table 43C, and/or Table 43D. The quantification data that is generated for these peptide structures may be input into a trained binary classification model to generate a disease indicator, which may be, for example, a probability score. A determination may be made as to whether the disease indicator (e.g., score) is above or below a selected threshold (e.g., 0.5). If the disease indicator is above the selected threshold, the biological sample may be classified as evidencing malignant pelvic tumor.

Further, this classification may further include a classification that the subject is in need of treatment. If the subject is in need of treatment based on the classification, treatment is administered. For example, a therapeutically effective amount of a therapeutic agent is administered to the patient, where the therapeutic agent is selected from a chemotherapeutic agent, an immunotherapeutic agent, a hormone therapy, a targeted therapeutic agent, a neoadjuvant therapy, or a combination.

### I.A. Exemplary Methodologies for Analyzing Peptide Structure Data and Managing Melanoma Treatment

### I.A.1. Predicting Treatment Response

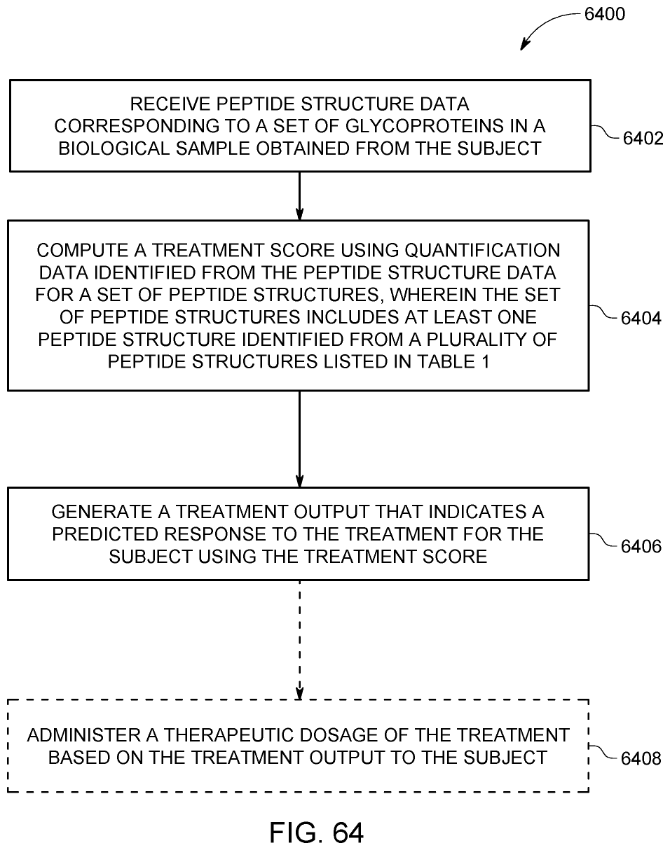

FIG. 64 is a flowchart of a process for managing a treatment for a subject diagnosed with a melanoma condition in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate, for example, a treatment output such as treatment output 334 in FIG. 3 to aid in the treatment of a subject diagnosed with a melanoma condition (e.g., malignant melanoma).

Step 502 includes receiving peptide structure data corresponding to a set of glycoproteins in a biological sample obtained from the subject. The peptide structure data may be, for example, one example of an implementation of peptide structure data 310 in FIG. 3. The peptide structure data may have been generated using multiple reaction monitoring mass spectrometry. The peptide structure data may include quantification data for each peptide structure of a plurality of peptide structures. The quantification data may include, for example, one or more quantification metrics for each peptide structure of the plurality of peptide structures. A quantification metric for a peptide structure may include, for example, but is not limited to, at least one of a relative abundance, an absolute abundance, an adjusted abundance, a normalized abundance, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample.

Step 504 includes computing a treatment score using quantification data identified from the peptide structure data for a set of peptide structures, wherein the set of peptide structures includes at least one peptide structure identified from a plurality of peptide structures listed in Table 55. In step 504, the set of peptide structures may include, for example, at least two peptide structures from a selected group of peptide structures identified in Table 55 below. The selected group of peptide structures may be, for example, a portion of the peptide structure identified in Table 55. The selected group of peptide structures may be, for example, those peptide structures identified in Table 56 below or those peptide structures identified in Table 57 below. For example, when the treatment being considered includes pembrolizumab, the selected group of peptide structures includes the peptide structures listed in Table 56. When the treatment being considered includes a combination of nivolumab and ipilimumab, the selected group of peptide structures includes the peptide structures listed in Table 57. In step 504, the set of peptide structures may include at least one glycopeptide structure defined by a peptide sequence and a glycan structure linked to a linking site of the peptide sequence, as identified in Table 55.

In one or more embodiments, the set of peptide structures may have been identified using sample data for a sample population (e.g., subjects diagnosed with melanoma in which at least a portion of the subjects have been treated using the treatment being considered in process 500) and a statistical algorithm that identifies a relative significance for each peptide structure of a collection of peptide structures corresponding to the sample data. The statistical algorithm may include, for example, a Wilcoxon rank-sum test. In one or more embodiments, the identification of the set of peptide structures is performed using process 800 described below in FIG. 8.

Step 504 may be performed by, for example, computing a proportion of the set of peptide structures having a certain type of abundance (e.g., relative abundance for glycopeptide structures and absolute abundance for aglycosylated peptide structures) greater than a reference abundance as the treatment score. In one or more embodiments, the reference abundance for a given peptide structure may be, for example, a median abundance of a plurality of abundances for that peptide structure across a sample population (e.g., as identified during training). The relative abundance for a given peptide structure is the abundance of that peptide structure relative to the corresponding aglycosylated peptide structure (e.g., the peptide structure having the same peptide sequence but without a glycan structure being bound to the peptide sequence).

Step 506 includes generating a treatment output that indicates a predicted response to the treatment for the subject using the treatment score. The treatment output may be one example of an implementation for treatment output 334 in FIG. 3. In one or more embodiments, step 506 may be performed by generating the predicted response to the treatment based on whether the treatment score is above a selected threshold. The selected threshold may be, for example, 0.5. For example, step 506 may include identifying a first predicted response classification for the subject when the treatment score is above 0.5 or identifying a second predicted response classification for the subject when the treatment score is not above 0. The first predicted response classification may be “sustained control” and the second predicted response classification may be “early disruption.” Sustained control may indicate that an absence of disruption events is predicted during a sustained period of time (e.g., 6 months) after treatment administration. Early disruption may indicate that a presence of at least one disruption event is predicted during an initial period of time (e.g., 12 months) after treatment.

The treatment outcome may include, for example, a recommendation to modify a treatment plan for the subject. For example, in some cases, the treatment output may indicate that early disruption is predicted for the subject. Accordingly, it may be desirable to modify the treatment plan. For example, the recommendation for modifying the treatment plan may include at least one of selecting a different treatment for the subject, alter (e.g., increase/decrease) a dosage for the treatment, or combining the treatment with at least one other treatment.

In one or more embodiments, the treatment output includes at least one of a design for the treatment or a therapeutic dosage for the treatment. For example, in some cases when the treatment score indicates that the subject will respond well (e.g., sustained control) to the treatment, the treatment outcome may identify the therapeutic dosage for the treatment. In this manner, a medical professional that receives the treatment output at a remote system (e.g., phone, tablet, laptop, etc.) may be able to more quickly administer the treatment to the subject.

In one or more embodiments, process 500 may optionally include step 508. Step 508 may include administering a therapeutic dosage of the treatment based on the treatment output to the subject. For example, the treatment may be administered (e.g., via intravenous or oral administration) based on the predicted response being a predicted response classification that indicates the treatment will be successful. For example, a predicted response classification of “sustained control” may indicate that the subject is predicted to respond well to treatment.

TABLE 55
Peptide Structures associated with Melanoma Treatments

| | | | | Mono- | Linking | Linking | |
|---|---|---|---|---|---|---|---|
| | Peptide | (Protein) | (Peptide) | isotopic | Site Pos. | Site Pos. | Glycan |
| PS-ID | Structure (PS) | SEQ ID | SEQ ID | mass | in Protein | in Peptide | Structure |
| NO. | NAME | NO. | NO. | (Da) | Sequence | Sequence | GL NO. |
| 330 | IGG1_297_5400 | 450 | 594 | 2811.09 | 180 | 5 | 5400 |
| 331 | IGG2_297_5411 | 451 | 595 | 3216.25 | 176 | 5 | 5411 |
| 332 | IGG1_297_5510 | 450 | 594 | 3160.22 | 180 | 5 | 5510 |
| 333 | IGG2_297_5410 | 451 | 595 | 2925.15 | 176 | 5 | 5410 |
| 334 | IGG1_297_5410 | 450 | 594 | 2957.14 | 180 | 5 | 5410 |
| 335 | IGG2_297_4411 | 451 | 595 | 3054.20 | 176 | 5 | 4411 |
| 336 | THBG_36_5402 | 452 | 593 | 3880.57 | 36 | 10 | 5402 |
| 337 | IGG2_297_5510 | 451 | 595 | 3128.23 | 176 | 5 | 5510 |
| 338 | AGP1_33_6503 | 453 | 570 | 5436.40 | 33 | 15 | 6503 |
| 339 | CO8B_243_6610 | 454 | 571 | 4231.67 | 243 | 11 | 6610 |
| 340 | IGA12_144_5502 | 455, 469 | 572 | 5370.44 | 144 | 18 | 5502 |
| 341 | KLKB1_494_5410 | 456 | 573 | 4014.82 | 494 | 6 | 5410 |
| 342 | IGG1_297_4400 | 450 | 594 | 2649.03 | 180 | 5 | 4400 |
| 343 | AACT_271_7602 | 457 | 574 | 4686.91 | 271 | 4 | 7602 |
| 344 | CO8B_553_5410 | 454 | 575 | 3454.29 | 553 | 6 | 5410 |
| 345 | FETUA_156_5402.5421 | 458 | 576 | 3975.61 | 156 | 12 | 5402 |
| 346 | IGA12_144_5501 | 455, 469 | 572 | 5079.35 | 144 | 18 | 5501 |
| 347 | IGG2_297_4500 | 451 | 595 | 2820.12 | 176 | 5 | 4500 |
| 348 | AGP1_33_6502 | 453 | 570 | 5145.31 | 33 | 15 | 6502 |
| 349 | CLUS_374_6520.6501 | 459 | 577 | 3961.64 | 374 | 3 | 6501 |
| 350 | A2MG_869_5200 | 460 | 578 | 4629.04 | 869 | 6 | 5200 |
| 351 | CFAH_882_5420.5401 | 461 | 579 | 3933.66 | 882 | 15 | 5401 |
| 352 | CFAH_911_5420.5401 | 461 | 580 | 3474.32 | 911 | 5 | 5401 |
| 353 | HEMO_453_5420.5401 | 462 | 581 | 3648.55 | 453 | 7 | 5401 |
| 354 | IGG34_297_4410 | 463, 468 | 582 | 2779.10 | 227 (IGG3)/ | 5 | 4410 |
| | | | | | 177 (IGG4) | | |
| 355 | KLKB1_127_5410 | 456 | 583 | 4014.82 | 127 | 5 | 5410 |
| 356 | TRFE_432_5401 | 464 | 584 | 3389.42 | 432 | 12 | 5401 |
| 357 | QUANTPEP.IGG4 | 468 | 585 | 1900.92 | N/A | N/A | N/A |
| | TTPPVLDSDGS | | | | | | |
| | FFL_YSR | | | | | | |
| 358 | NEWQUANTPEP- | 463 | 586 | 2413.15 | N/A | N/A | N/A |
| | IGG3_TPEVTCV | | | | | | |
| | VVDVSHEDPEV | | | | | | |
| | QFK | | | | | | |
| 359 | A2MG_869_6200 | 460 | 578 | 4791.10 | 869 | 6 | 6200 |
| 360 | HPT_184_5511 | 465 | 587 | 4941.20 | 184 | 6 | 5511 |
| 361 | VTNC_169_5401 | 466 | 588 | 2824.14 | 169 | 1 | 5401 |
| 362 | AACT_271_7603 | 457 | 574 | 4978.01 | 271 | 4 | 7603 |
| 363 | HPT_207_10803 | 465 | 589 | 5576.18 | 207 & 211 | 5 & 9 | 5401 & |
| | | | | | | | 5402 |
| 364 | HPT_241_5401.5420 | 465 | 590 | 3707.68 | 241 | 6 | 5401 |
| 365 | IGG34_297_4411 | 463, 468 | 582 | 3070.19 | 227 (IGG3)/ | 5 | 4411 |
| | | | | | 177 (IGG4) | | |
| 366 | ITIH4_517_5420.5401 | 467 | 591 | 4722.02 | 517 | 5 | 5401 |
| 367 | AACT_127_5401 | 457 | 592 | 4125.73 | 127 | 3 | 5401 |

TABLE 56
Peptide Structures associated with a First Treatment (e.g., Pembrolizumab Tx)

| | Peptide | | | Mono- | Linking | Linking | |
|---|---|---|---|---|---|---|---|
| | Structure | (Protein) | (Peptide) | isotopic | Site Pos. | Site Pos. | Glycan |
| PS-ID | (PS) | SEQ ID | SEQ ID | mass | in Protein | in Peptide | Structure |
| NO. | NAME | NO. | NO. | (Da) | Sequence | Sequence | GL NO. |
| 330 | IGG1_297_5400 | 450 | 594 | 2811.09 | 180 | 5 | 5400 |
| 331 | IGG2_297_5411 | 451 | 595 | 3216.25 | 176 | 5 | 5411 |
| 332 | IGG1_297_5510 | 450 | 594 | 3160.22 | 180 | 5 | 5510 |
| 333 | IGG2_297_5410 | 451 | 595 | 2925.15 | 176 | 5 | 5410 |
| 334 | IGG1_297_5410 | 450 | 594 | 2957.14 | 180 | 5 | 5410 |
| 335 | IGG2_297_4411 | 451 | 595 | 3054.20 | 176 | 5 | 4411 |
| 336 | THBG_36_5402 | 452 | 593 | 3880.57 | 36 | 10 | 5402 |
| 337 | IGG2_297_5510 | 451 | 595 | 3128.23 | 176 | 5 | 5510 |
| 338 | AGP1_33_6503 | 453 | 570 | 5436.40 | 33 | 15 | 6503 |
| 339 | CO8B_243_6610 | 454 | 571 | 4231.67 | 243 | 11 | 6610 |
| 340 | IGA12_144_5502 | 455, 469 | 572 | 5370.44 | 144 | 18 | 5502 |
| 341 | KLKB1_494_5410 | 456 | 573 | 4014.82 | 494 | 6 | 5410 |
| 342 | IGG1_297_4400 | 450 | 594 | 2649.03 | 180 | 5 | 4400 |
| 343 | AACT_271_7602 | 457 | 574 | 4686.91 | 271 | 4 | 7602 |
| 344 | CO8B_553_5410 | 454 | 575 | 3454.29 | 553 | 6 | 5410 |
| 345 | FETUA_156_5402.5421 | 458 | 576 | 3975.61 | 156 | 12 | 5402 |
| 346 | IGA12_144_5501 | 455, 469 | 572 | 5079.35 | 144 | 18 | 5501 |
| 347 | IGG2_297_4500 | 451 | 595 | 2820.12 | 176 | 5 | 4500 |
| 348 | AGP1_33_6502 | 453 | 570 | 5145.31 | 33 | 15 | 6502 |
| 349 | CLUS_374_6520.6501 | 459 | 577 | 3961.64 | 374 | 3 | 6501 |

TABLE 57
Peptide Structures associated with a Second treatment
(e.g., Ipilimumab/Nivolumab Tx)

| | | | | Mono- | Linking | Linking | |
|---|---|---|---|---|---|---|---|
| | | (Protein) | (Peptide) | isotopic | Site Pos. | Site Pos. | Glycan |
| PS-ID | Peptide Structure | SEQ ID | SEQ ID | mass | in Protein | in Peptide | Structure |
| NO. | (PS) NAME | NO. | NO. | (Da) | Sequence | Sequence | GL NO. |
| 350 | A2MG_869_5200 | 460 | 578 | 4629.04 | 869 | 6 | 5200 |
| 338 | AGP1_33_6503 | 453 | 570 | 5436.40 | 33 | 15 | 6503 |
| 351 | CFAH_882_5420.5401 | 461 | 579 | 3933.66 | 882 | 15 | 5401 |
| 352 | CFAH_911_5420.5401 | 461 | 580 | 3474.32 | 911 | 5 | 5401 |
| 353 | HEMO_453_5420.5401 | 462 | 581 | 3648.55 | 453 | 7 | 5401 |
| 354 | IGG34_297_4410 | 463, 468 | 582 | 2779.10 | 227 (IGG3)/ | 5 | 4410 |
| | | | | | 177 (IGG4) | | |
| 355 | KLKB1_127_5410 | 456 | 583 | 4014.82 | 127 | 5 | 5410 |
| 356 | TRFE_432_5401 | 464 | 584 | 3389.42 | 432 | 12 | 5401 |
| 357 | QUANTPEP.IGG4 | 468 | 585 | 1900.92 | N/A | N/A | N/A |
| | TTPPVLDSDGSFF | | | | | | |
| | LYSR | | | | | | |
| 358 | NEWQUANTPEP- | 463 | 586 | 2413.15 | N/A | N/A | N/A |
| | IGG3_TPEVTCVV | | | | | | |
| | VDVSHEDPEVQFK | | | | | | |
| 359 | A2MG_869_6200 | 460 | 578 | 4791.10 | 869 | 6 | 6200 |
| 360 | HPT_184_5511 | 465 | 587 | 4941.20 | 184 | 6 | 5511 |
| 361 | VTNC_169_5401 | 466 | 588 | 2824.14 | 169 | 1 | 5401 |
| 362 | AACT_271_7603 | 457 | 574 | 4978.01 | 271 | 4 | 7603 |
| 363 | HPT_207_10803 | 465 | 589 | 5576.18 | 207 & 211 | 5 & 9 | 5401 & |
| | | | | | | | 5402 |
| 364 | HPT_241_5401.5420 | 465 | 590 | 3707.68 | 241 | 6 | 5401 |
| 365 | IGG34_297_4411 | 463, 468 | 582 | 3070.19 | 227 (IGG3)/ | 5 | 4411 |
| | | | | | 177 (IGG4) | | |
| 366 | ITIH4_517_5420.5401 | 467 | 591 | 4722.02 | 517 | 5 | 5401 |
| 341 | KLKB1_494_5410 | 456 | 573 | 4014.82 | 494 | 6 | 5410 |
| 367 | AACT_127_5401 | 457 | 592 | 4125.73 | 127 | 3 | 5401 |

TABLE 58A.1
Peptide Structures associated with Progression of Disease (NSCLC)

| | | | | Mono- | Linking | Linking | |
|---|---|---|---|---|---|---|---|
| PS- | Peptide | Protein | | isotopic | Site Pos. | Site Pos. | Glycan |
| ID | Structure (PS) | UniProt | Peptide | mass | in Protein | in Peptide | Structure |
| NO. | NAME | ID | Sequence | (Da) | Sequence | Sequence | GL NO. |
| 368 | VTNC_242_6502 | P04004 | NISDGFDGIP | 5341.2196 | 242 | 1 | 6502 |
| | | | DNVDAALAL | | | | |
| | | | PAHSYSGR | | | | |
| 369 | THRB_416MC_5402 | P00734 | WVLTAAHCL | 5375.373484 | 416 | 17 | 5402 |
| | | | LYPPWDKNF | | | | |
| | | | TENDLLVR | | | | |
| 370 | QUANTPEP.AACT_ | P01011 | ADLSGITGAR | 959.503584 | N/A | N/A | N/A |
| | ADLSGITGAR | | | | | | |
| 371 | QUANTPEP.I | P05155 | GVTSVSQIFHS | 1825.968604 | N/A | N/A | N/A |
| | C1_GVTSVSQ | | PDLAIR | | | | |
| | IFHSPDLAIR | | | | | | |
| 372 | HPT_241_6511 | P00738 | VVLHPNYSQ | 4218.871006 | 241 | 6 | 6511 |
| | | | VDIGLIK | | | | |
| 373 | VTNC_169_6301 | P04004 | NGSLFAFR | 2783.116514 | 169 | 1 | 6301 |
| 374 | ITIHI_653_1102 | P19827 | TFVLSALQPS | 3104.4044 | 653 | 12 | 1102 |
| | | | PTHSSSNTQR | | | | |
| 375 | FETUA_176_6513 | P02765 | AALAAFNAQ | 5371.203674 | 176 | 11 | 6513 |
| | | | NNGSNFQLE | | | | |
| | | | EISR | | | | |
| 376 | A1AT_70_5402 | P01009 | QLAHQSNST | 5385.40009 | 70 | 7 | 5402 |
| | | | NIFFSPVSIAT | | | | |
| | | | AFAMLSLGT | | | | |
| | | | K | | | | |
| 377 | QUANTPEP.C | P13671 | IGESIELTCPK | 1245.62746 | N/A | N/A | N/A |
| | 06_IGESIELT | | | | | | |
| | CPK | | | | | | |
| 378 | IGM_46_6311 | P01871 | YKNNSDISST | 3302.3189 | 46 | 3 | 6311 |
| | | | R | | | | |
| 379 | APOM_135_5402 | 095445 | TELFSSSCPG | 4735.914404 | 135 | 15 | 5402 |
| | | | GIMLNETGQ | | | | |
| | | | GYQR | | | | |
| 380 | IC1_238_5412 | P05155 | DTFVNASR | 3259.26547 | 238 | 6 | 5412 |
| 381 | NEWQUANTP | P01860 | TPEVTCVVV | 2413.147086 | N/A | N/A | N/A |
| | EP.IGG3_TPE | | DVSHEDPEV | | | | |
| | VTCVVVDVS | | QFK | | | | |
| | HEDPEVQFK | | | | | | |
| 382 | A1AT_70_NO | P01009 | QLAHQSNST | 3180.627698 | N/A | N/A | N/A |
| | NGLYCOSYL | | NIFFSPVSIAT | | | | |
| | ATED | | AFAMLSLGT | | | | |
| | | | K | | | | |
| 383 | HPT_184_5402 | P00738 | MVSHHNLTT | 4883.15735 | 184 | 6 | 5402 |
| | | | GATLINEQW | | | | |
| | | | LLTTAK | | | | |
| 384 | QUANTPEP.C05_ | P01031 | GIYGTISR | 865.465746 | N/A | N/A | N/A |
| | GIYGTISR | | | | | | |
| 385 | KNG1_205_5402 | P01042 | ITYSIVQTNC | 3617.469324 | 205 | 9 | 5402 |
| | | | SK | | | | |
| 386 | IGG2_297_3310 | P01859 | EEQFNSTFR | 2397.969344 | 176 | 5 | 3310 |
| 387 | IGM_209_5500 | P01871 | GLTFQQNAS | 4163.729062 | 209 | 7 | 5500 |
| | | | SMCVPDQDT | | | | |
| | | | AIR | | | | |

TABLE 58A.2
PS-ID NO., Protein Sequence ID NO., and Peptide Sequence ID No. of the
Peptide Structures associated with Progression of Disease (NSCLC)

| PS- | | Protein | |
|---|---|---|---|
| ID | | SEQ ID | Peptide SEQ |
| NO. | Peptide Structure (PS) NAME | NO | ID NO |
| 368 | VTNC_242_6502 | 593 | 613 |
| 369 | THRB_416MC_5402 | 594 | 614 |
| 370 | QUANTPEP.AACT_ADLSGITGAR | 595 | 615 |
| 371 | QUANTPEP.ICI_GVTSVSQIFH | 596 | 616 |
| | SPDLAIR | | |
| 372 | HPT_241_6511 | 597 | 617 |
| 373 | VTNC_169_6301 | 598 | 618 |
| 374 | ITIH1_653_1102 | 599 | 619 |
| 375 | FETUA_176_6513 | 600 | 620 |
| 376 | A1AT_70_5402 | 601 | 621 |
| 377 | QUANTPEP.CO6_IGESIELTCPK | 602 | 622 |
| 378 | IGM_46_6311 | 603 | 623 |
| 379 | APOM_135_5402 | 604 | 624 |
| 380 | IC1_238_5412 | 605 | 625 |
| 381 | NEWQUANTPEP.IGG3_TPEVT | 606 | 626 |
| | CVVVDVSHEDPEVQFK | | |
| 382 | ALAT_70_NONGLYCOSYLATED | 607 | 627 |
| 383 | HPT_184_5402 | 608 | 628 |
| 384 | QUANTPEP.CO5_GIYGTISR | 609 | 629 |
| 385 | KNG1_205_5402 | 610 | 630 |
| 386 | IGG2_297_3310 | 611 | 631 |
| 387 | IGM_209_5500 | 612 | 632 |

TABLE 58B.1
Peptide Structures associated with Death (NSCLC)

| | | | | Mono- | Linking | Linking | |
|---|---|---|---|---|---|---|---|
| | | (Protein) | | isotopic | Site Pos. | Site Pos. | Glycan |
| PS-ID | Peptide Structure | UniProt | Peptide | mass | in Protein | in Peptide | Structure |
| NO. | (PS) NAME | ID | Sequence | (Da) | Sequence | Sequence | GL NO. |
| 388 | QUANTPEP.AAC | P01011 | ADLSGI | 959.503584 | N/A | N/A | N/A |
| | T_ADLSGITGAR | | TGAR | | | | |
| 389 | APOM_135_5402 | 095445 | TELFSS | 4735.914404 | 135 | 15 | 5402 |
| | | | SCPGGI | | | | |
| | | | MLNET | | | | |
| | | | GQGYQ | | | | |
| | | | R | | | | |
| 390 | PEP.AGP1_YVG | P02763 | YVGGQ | 1751.947084 | N/A | N/A | N/A |
| | GQEHFAHLLIL | | EHFAH | | | | |
| | R | | LLILR | | | | |
| 391 | IC1_238_5412 | P05155 | DTFVN | 3259.26547 | 238 | 6 | 5412 |
| | | | ASR | | | | |
| 392 | PEP.AGP12_TED | P02763 | TEDTIF | 993.513088 | N/A | N/A | N/A |
| | TIFLR | or | LR | | | | |
| | | P19652 | | | | | |
| 393 | QUANTPEP.CO5 | P01031 | GIYGTI | 865.465746 | N/A | N/A | N/A |
| | GIYGTISR | | SR | | | | |
| 394 | FETUA_176_6502 | P02765 | AALAA | 4934.050358 | 176 | 11 | 6502 |
| | | | FNAQN | | | | |
| | | | NGSNF | | | | |
| | | | QLEEIS | | | | |
| | | | R | | | | |
| 395 | FETUA_176_6513 | P02765 | AALAA | 5371.203674 | 176 | 11 | 6513 |
| | | | FNAQN | | | | |
| | | | NGSNF | | | | |
| | | | QLEEIS | | | | |
| | | | R | | | | |
| 396 | QUANTPEP.CO6 | P13671 | IGESIEL | 1245.62746 | N/A | N/A | N/A |
| | IGESIELTCPK | | TCPK | | | | |
| 397 | HPT_241_6511 | P00738 | VVLHP | 4218.871006 | 241 | 6 | 6511 |
| | | | NYSQV | | | | |
| | | | DIGLIK | | | | |
| 398 | APOB_3895_5401 | P04114 | FEVDSP | 3826.597634 | 3895 | 9 | 5401 |
| | | | VYNAT | | | | |
| | | | WSASL | | | | |
| | | | K | | | | |
| 399 | QUANTPEP.HPT | P00738 | ILGGHL | 922.523594 | N/A | N/A | N/A |
| | ILGGHLDAK | | DAK | | | | |
| 400 | HPT_184_5402 | P00738 | MVSHH | 4883.15735 | 184 | 6 | 5402 |
| | | | NLTTG | | | | |
| | | | ATLINE | | | | |
| | | | QWLLT | | | | |
| | | | TAK | | | | |
| 401 | ALAT_70_NONG | P01009 | QLAHQ | 3180.627698 | N/A | N/A | N/A |
| | LYCOSYLATED | | SNSTNI | | | | |
| | | | FFSPVSI | | | | |
| | | | ATAFA | | | | |
| | | | MLSLG | | | | |
| | | | TK | | | | |
| 402 | IGM_439_6200 | P01871 | STGKPT | 3742.57335 | 440 | 9 | 6200 |
| | | | LYNVS | | | | |
| | | | LVMSD | | | | |
| | | | TAGTC | | | | |
| | | | Y | | | | |
| 403 | QUANTPEP.IC1 | P05155 | GVTSVS | 1825.968604 | N/A | N/A | N/A |
| | GVTSVSQIFHSP | | QIFHSP | | | | |
| | DLAIR | | DLAIR | | | | |
| 404 | APOH_162_5412 | P02749 | VYKPS | 3818.57729 | 162 | 8 | 5412 |
| | | | AGNNS | | | | |
| | | | LYR | | | | |
| 405 | FETUA_176_6501 | P02765 | AALAA | 4642.954948 | 176 | 11 | 6501 |
| | | | FNAQN | | | | |
| | | | NGSNF | | | | |
| | | | QLEEIS | | | | |
| | | | R | | | | |
| 406 | KLKB1_127_5402 | P03952 | GVNFN | 3068.222488 | 127 | 5 | 5402 |
| | | | VSK | | | | |
| 407 | VTNC_169_6301 | P04004 | NGSLFA | 2783.116514 | 169 | 1 | 6301 |
| | | | FR | | | | |

TABLE 58B.2
PS-ID NO., Protein Sequence ID NO., and Peptide Sequence ID No.
of the Peptide Structures associated with Death (NSCLC)

| | | Protein | Peptide |
|---|---|---|---|
| PS-ID | | SEQ ID | SEQ ID |
| NO. | Peptide Structure (PS) NAME | NO | NO |
| 388 | QUANTPEP.AACT_ADLSGITGAR | 633 | 653 |
| 389 | APOM_135_5402 | 634 | 654 |
| 390 | PEP.AGP1_YVGGQEHFAHLLILR | 635 | 655 |
| 391 | IC1_238_5412 | 636 | 656 |
| 392 | PEP.AGP12_TEDTIFLR | 635 or | 657 |
| | | 637 | |
| 393 | QUANTPEP.CO5_GIYGTISR | 638 | 658 |
| 394 | FETUA_176_6502 | 639 | 659 |
| 395 | FETUA_176_6513 | 640 | 660 |
| 396 | QUANTPEP.CO6_IGESIELTCPK | 641 | 661 |
| 397 | HPT_241_6511 | 642 | 662 |
| 398 | APOB_3895_5401 | 643 | 663 |
| 399 | QUANTPEP.HPT_ILGGHLDAK | 644 | 664 |
| 400 | HPT_184_5402 | 645 | 665 |
| 401 | A1AT_70_NONGLYCOSYLATED | 646 | 666 |
| 402 | IGM_439_6200 | 647 | 667 |
| 403 | QUANTPEP.ICI_GVTSVSQIFHSPDLAIR | 648 | 668 |
| 404 | APOH_162_5412 | 649 | 669 |
| 405 | FETUA_176_6501 | 650 | 670 |
| 406 | KLKB1_127_5402 | 651 | 671 |
| 407 | VTNC_169_6301 | 652 | 672 |

### I.A.2. Selecting Between Multiple Treatments

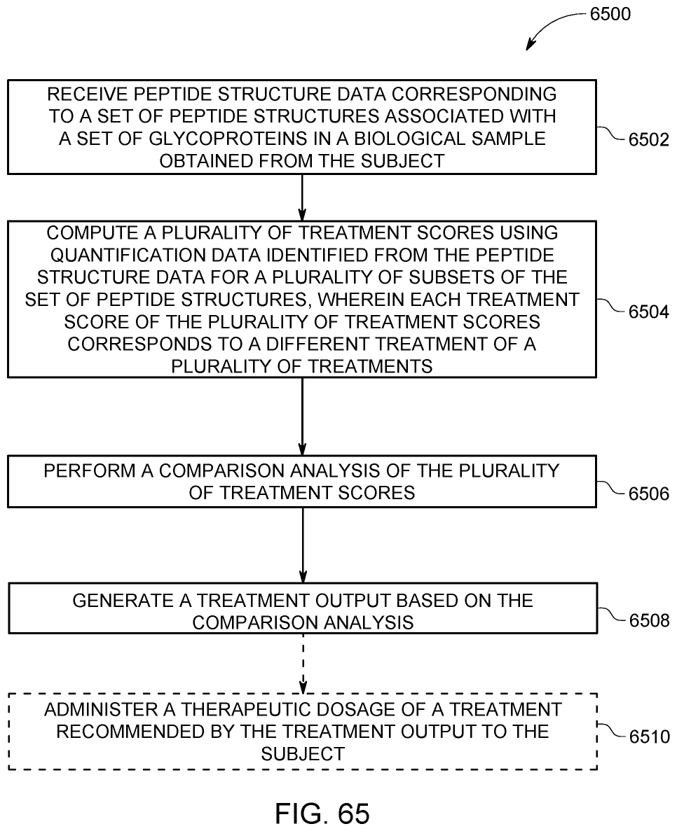

FIG. 65 is a flowchart of a process for treatment management of a subject diagnosed with a melanoma condition in accordance with various embodiments. Process 600 may be implemented using, for example, at least a portion of workflow 100 as described in FIGS. 1, 2A, and 2B and/or analysis system 300 as described in FIG. 3. In some embodiments, process 600 may be one example that includes and expands upon process 500 in FIG. 64.

Step 602 may include receiving peptide structure data corresponding to a set of peptide structures associated with a set of glycoproteins in a biological sample obtained from the subject. Step 602 may be performed in a manner similar to step 502 as described above with respect to FIG. 64.

Step 604 may include computing a plurality of treatment scores using quantification data identified from the peptide structure data for a plurality of subsets of the set of peptide structures, wherein each treatment score of the plurality of treatment scores corresponds to a different treatment of a plurality of treatments. Each subset of the plurality of subsets may include at least one peptide structure identified from a plurality of peptide structures listed in Table 55. Computing a treatment score of the plurality of treatment scores may be performed in a manner similar to step 504 as described above with respect to FIG. 64. Each treatment score may be computed using, for example, a proportion of a subset of the plurality of subsets of the set of peptide structures having a selected abundance (e.g., relative abundance for glycopeptide structures and absolute abundance for aglycosylated peptide structures) greater than a reference abundance for that peptide structure as a treatment score of the plurality of treatment scores.

In one or more embodiments, the plurality of subsets includes a first subset and a second subset. For example, step 604 may include computing a first treatment score for a first treatment of using a first portion of the quantification data identified from the peptide structure data for a first subset of the plurality of subsets of the set of peptide structures. Step 604 may further include computing a second treatment score for the second treatment using a second portion of the quantification data identified from the peptide structure data for a second subset of the plurality of subsets of the set of peptide structures. The first subset may include one or more peptide structures from those listed in Table 56. The second subset may include one or more peptide structures from those listed in Table 57.

In one or more embodiments, a subset of the plurality of subsets may have been previously identified using sample data for a sample population (e.g., subjects diagnosed with melanoma, in which at least a portion of the sample population has been treated with the plurality of treatments) and a statistical algorithm that identifies a relative significance for each peptide structure of a collection of peptide structures corresponding to the sample data with respect to a response to a selected treatment of the plurality of treatments. For example, identifying the subset may include performing a differential abundance analysis using the sample data to compare a first portion of the sample data corresponding to a first response classification (e.g., a positive response classification such as, for example, sustained control) for the selected treatment and a second portion of the sample data corresponding to a second response classification (e.g., a negative response classification such as, for example, early disruption) for the selected treatment to identify a selected N most differentiating peptide structures (e.g., the 20 most differentiating peptide structures) between the first response classification and the second response classification. The statistical algorithm may include, for example, a Wilcoxon rank-sum test.

Step 606 may include performing a comparison analysis of the plurality of treatment scores. Step 606 may be performed by, for example, determining which of the plurality of treatment scores is a highest-scoring treatment score. In some embodiments, step 606 may include determining that a treatment of the plurality of treatments has a treatment score below a selected threshold and excluding that treatment from the comparison analysis. The selected threshold may be, for example, 0.5.

Step 608 may include generating a treatment output based on the comparison analysis. The treatment output includes a recommended treatment plan for treating the subject. For example, step 608 may include identifying the treatment of the plurality of treatments having a highest treatment score as a recommended treatment for treating the subject.

In one or more embodiments, step 608 may include identifying a predicted response classification for the subject for each treatment of the plurality of treatments using a corresponding treatment score of the plurality of treatment scores. The predicted response classification may be, for example, a positive response classification, a negative response classification, or another type of response classification. In one or more embodiments, the predicted response classification for a particular treatment may be, for example, sustained control when the corresponding treatment score is above a selected threshold and may be, for example, early disruption when the corresponding treatment score is not above the selected threshold. The selected threshold may be, for example, 0.5.

In one or more embodiments, step 608 includes identifying a treatment of the plurality of treatments having a highest treatment score as a highest-scored treatment; determining that the highest treatment score is not above a selected threshold (e.g., 0.5); and generating the treatment output such that the recommended treatment plan includes a recommendation to modify an existing treatment plan for the subject. The recommendation for modifying the treatment plan may include at least one of selecting a different treatment for the subject, altering a dosage for a treatment that is part of the existing treatment plan, or combining the treatment with at least one other treatment.

In one or more embodiments, when the treatment output includes a recommended treatment, process 600 may optionally include step 610. Step 610 may include administering a therapeutic dosage of a treatment recommended by the treatment output to the subject.

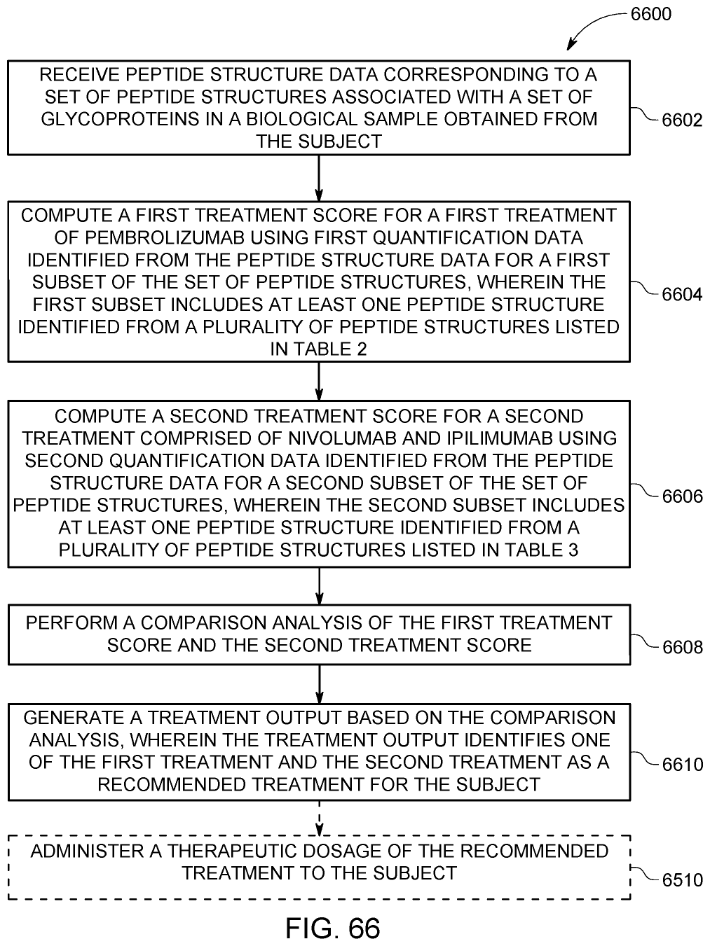

FIG. 66 is a flowchart of a process for treatment management of a subject diagnosed with a melanoma condition in accordance with various embodiments. Process 700 may be implemented using, for example, at least a portion of workflow 100 as described in FIGS. 1, 2A, and 2B and/or analysis system 300 as described in FIG. 3. In some embodiments, process 700 may be one example that includes and expands upon process 500 in FIG. 64. Further, process 700 may be one example of an implementation of process 600 in FIG. 65.

Step 702 may include receiving peptide structure data corresponding to a set of peptide structures associated with a set of glycoproteins in a biological sample obtained from the subject. Step 702 may be performed in a manner similar to step 502 as described above with respect to FIG. 64.

Step 704 may include computing a first treatment score for a first treatment of pembrolizumab using first quantification data identified from the peptide structure data for a first subset of the set of peptide structures, wherein the first subset includes at least one peptide structure identified from a plurality of peptide structures listed in Table 56. The treatment score may be computed using, for example, a proportion of a subset of the plurality of subsets of the set of peptide structures having a selected abundance (e.g., relative abundance for glycopeptide structures and absolute abundance for aglycosylated peptide structures) greater than a reference abundance for that peptide structure as a treatment score of the plurality of treatment scores. In one or more embodiments, the first subset includes all of or a majority of (e.g., more than 15) the peptide structures listed in Table 56.

Step 706 may include computing a second treatment score for a second treatment comprised of nivolumab and ipilimumab using second quantification data identified from the peptide structure data for a second subset of the set of peptide structures, wherein the second subset includes at least one peptide structure identified from a plurality of peptide structures listed in Table 57. In one or more embodiments, the first subset includes all of or a majority of (e.g., more than 15) the peptide structures listed in Table 57.

Step 708 may include performing a comparison analysis of the first treatment score and the second treatment score. Step 708 may include, for example, determining which of the first treatment score and the second treatment score is a highest score.

Step 710 may include generating a treatment output based on the comparison analysis, wherein the treatment output identifies one of the first treatment and the second treatment as a recommended treatment for the subject. For example, step 710 may include identifying the highest-scoring treatment as a recommended treatment for treating the subject. The recommended treatment may then be administered to the subject to treat the subject's melanoma. For example, the treatment may be administered via at least one of intravenous or oral administration at a therapeutic dosage.

In one or more embodiments, process 700 may optionally include step 712. Step 712 may include administering a therapeutic dosage of the recommended treatment to the subject.

### I.B. Exemplary Methodology for Identifying a Set of Peptide Structures Corresponding to a Treatment

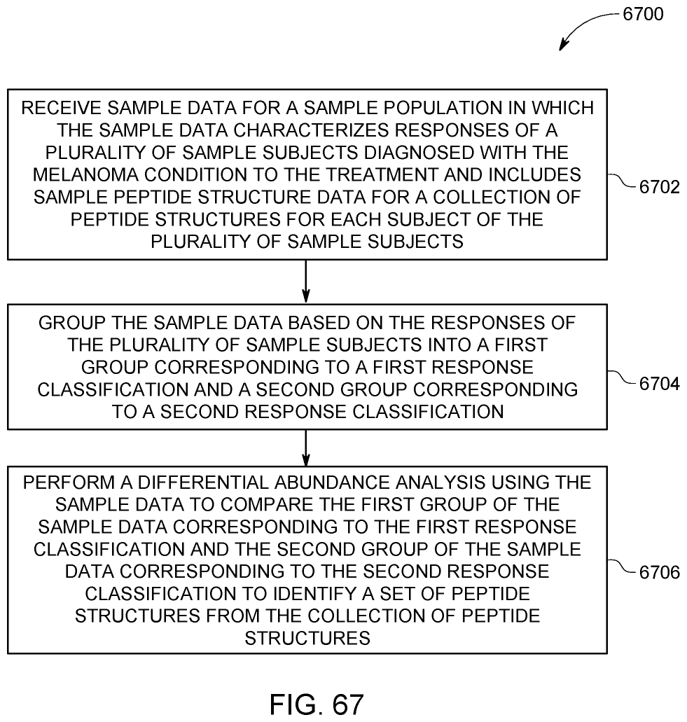

FIG. 67 is a flowchart of a process for identifying a treatment for a subject diagnosed with a melanoma condition in accordance with one or more embodiments. Process 800 may be implemented using, for example, at least a portion of workflow 100 as described in FIGS. 1, 2A, and 2B and/or analysis system 300 as described in FIG. 3. In some embodiments, process 800 may be one example that includes and expands upon process 500 in FIG. 64.

Step 802 includes receiving sample data for a sample population in which the sample data characterizes responses of a plurality of sample subjects diagnosed with the melanoma condition to the treatment and includes sample peptide structure data for a collection of peptide structures for each subject of the plurality of sample subjects.

Step 804 includes grouping the sample data based on the responses of the plurality of sample subjects into a first group corresponding to a first response classification and a second group corresponding to a second response classification.

Step 806 includes performing a differential abundance analysis using the sample data to compare the first group of the sample data corresponding to the first response classification and the second group of the sample data corresponding to the second response classification to identify a set of peptide structures from the collection of peptide structures. The set of peptide structures may be identified as a selected N most differentiating peptide structures (e.g., the 20 most significant peptide structures for differentiation) between the first response classification and the second response classification. The first response classification may be, for example, sustained control, which indicates an absence of disruption events during a sustained period of time (e.g., 12 months) after treatment administration. The second response classification may be, for example, early disruption, which indicates a presence of at least one disruption event during an initial period of time (e.g., 6 months) after treatment.

This set of peptide structure that is identified in step 806 may then be used in future analysis (e.g., in process 500 in FIG. 64, in process 600 in FIG. 65, in process 700 in FIG. 66) to compute a treatment score for a subject using the subject's peptide structure profile that indicates the likelihood of a successful response (e.g., sustained control) of the subject to the treatment.

Step 806 may be performed using, for example, a Wilcoxon rank-sum test in one or more embodiments. Exemplary results of the differential abundance analysis performed using the Wilcoxon rank-sum test are presented below in Table 59A and Table 59B.

TABLE 59A
Wilcoxon Analysis of Peptide Structures associated
with Pembrolizumab Tx

| PS- | | | | | |
|---|---|---|---|---|---|
| ID | Median | Median | Differential | Wilcoxon | |
| NO. | SC | EF | (SC-EF) | p-value | FDR |
| 330 | 0.5016406 | −0.3477531 | 0.8493937 | 0.0017802 | 0.3761093 |
| 331 | 0.5490382 | −0.6903325 | 1.2393706 | 0.0022447 | 0.3761093 |
| 332 | 0.6102916 | −0.4022977 | 1.0125893 | 0.0028112 | 0.3761093 |
| 333 | 0.4726799 | −0.8630625 | 1.3357424 | 0.0034924 | 0.3761093 |
| 334 | 0.9085908 | −0.820044 | 1.7286347 | 0.0043126 | 0.3761093 |
| 335 | −0.0540671 | −0.3156836 | 0.2616165 | 0.0052867 | 0.3761093 |
| 336 | 0.2843746 | −0.31304 | 0.5974146 | 0.0052867 | 0.3761093 |
| 337 | 0.3041313 | −0.568187 | 0.8723183 | 0.0064434 | 0.4011026 |
| 338 | 0.3805894 | −0.3185274 | 0.6991168 | 0.0078028 | 0.4317552 |
| 339 | 0.6412248 | −0.2431649 | 0.8843898 | 0.0093974 | 0.4679899 |
| 340 | −0.0136785 | −0.6949529 | 0.6812744 | 0.0112501 | 0.5093223 |
| 341 | 0.2518882 | −0.4929206 | 0.7448088 | 0.0134001 | 0.5561048 |
| 342 | 0.6384324 | −0.1801925 | 0.8186249 | 0.0158719 | 0.582317 |
| 343 | 0.3603753 | −0.1926793 | 0.5530546 | 0.018709 | 0.582317 |
| 344 | 0.5414354 | −0.2145807 | 0.7560161 | 0.018709 | 0.582317 |
| 345 | −0.0702782 | −0.4763048 | 0.4060266 | 0.018709 | 0.582317 |
| 346 | 0.4330799 | −0.4610782 | 0.8941581 | 0.0219396 | 0.6069946 |
| 347 | 0.2377877 | −0.5018914 | 0.7396791 | 0.0219396 | 0.6069946 |
| 348 | 0.4095444 | −0.313772 | 0.7233164 | 0.029749 | 0.6590555 |
| 349 | 0.1573811 | −0.2217593 | 0.3791404 | 0.029749 | 0.6590555 |

TABLE 59B
Wilcoxon Analysis of Peptide Structures associated with
Ipilimumab/Nivolumab Tx

| PS- | | | | | |
|---|---|---|---|---|---|
| ID | Median | Median | Differential | Wilcoxon | |
| NO. | SC | EF | (SC-EF) | p-value | FDR |
| 350 | 0.3328389 | −0.6312886 | 0.9641274 | 0.0021645 | 0.3761093 |
| 338 | 0.5024846 | 0.0758823 | 0.4266023 | 0.0021645 | 0.3761093 |
| 351 | 1.0534081 | −0.6860991 | 1.7395073 | 0.0021645 | 0.3761093 |
| 352 | 0.7030683 | −0.5793093 | 1.2823776 | 0.0021645 | 0.3761093 |
| 353 | 0.5131039 | −0.792533 | 1.3056369 | 0.0021645 | 0.3761093 |
| 354 | 0.4540561 | −0.9637756 | 1.4178318 | 0.0021645 | 0.3761093 |
| 355 | 0.6041198 | −0.8676916 | 1.4718114 | 0.0021645 | 0.3761093 |
| 356 | 0.3696252 | −0.8139757 | 1.1836009 | 0.0021645 | 0.4011026 |
| 357 | 1.0638627 | −1.0730903 | 2.1369529 | 0.0021645 | 0.4317552 |
| 358 | 0.938314 | −1.056397 | 1.994711 | 0.0021645 | 0.4679899 |
| 359 | 0.1958926 | −0.7169942 | 0.9128868 | 0.004329 | 0.5093223 |
| 360 | 0.3090463 | −1.5388815 | 1.8479278 | 0.004329 | 0.5561048 |
| 361 | 0.9161205 | −0.7184875 | 1.634608 | 0.004329 | 0.582317 |
| 362 | 0.1694553 | −1.6309309 | 1.8003861 | 0.008658 | 0.582317 |
| 363 | 0.3946123 | −0.5476397 | 0.942252 | 0.0151515 | 0.582317 |
| 364 | 0.320616 | −0.4720598 | 0.7926757 | 0.0151515 | 0.582317 |
| 365 | 0.4591413 | −0.6433692 | 1.1025105 | 0.0151515 | 0.6069946 |
| 366 | 0.0750044 | −1.5985227 | 1.6735272 | 0.0151515 | 0.6069946 |
| 341 | 0.3832391 | −0.6207699 | 1.0040091 | 0.0151515 | 0.6590555 |
| 367 | 0.6264716 | 0.1222803 | 0.5041913 | 0.025974 | 0.6590555 |

### VIII. Peptide Structure and Product Ion Compositions, Kits, and Reagents

Aspects of the disclosure include compositions comprising one or more of the peptide structures listed in Table 1A or 1B. In some embodiments, a composition comprises a plurality of the peptide structures listed in Table 1A or 1B. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the peptide structures listed in Table 1A. In some embodiments, a composition comprises 1, 2, 3, 4, 5, 6, 7, 8, 9, 10, 11, 12, 13, or 14 of the peptide structures listed in Table 1B. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 1-23, listed in Table 1A. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 1-11, listed in Table 1B.

Aspects of the disclosure include compositions comprising one or more precursor ions having a defined charge and/or defined mass-to-charge (m/z) ratio, as listed in Table 4 below. Aspects of the disclosure include compositions comprising one or more product ions having a defined mass-to-charge (m/z) ratio, which product ions are produced by converting a peptide structure described herein (e.g., a peptide structure listed in Table 1A or 1B) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1A or 1B). In some embodiments, a composition comprises a set of the product ions listed in Table 4, having an m/z ratio selected from the list provided for each peptide structure in Table 4.

In some embodiments, a composition comprises at least one of peptide structures identified in Table 1A or 1B.

In some embodiments, a composition comprises a peptide structure or a product ion. The peptide structure or product ion comprises an amino acid sequence having at least 90% sequence identity to any one of SEQ ID NOS: 1-23, as identified in Table 5A below, corresponding to peptide structures in Table 1A.

In some embodiments, the product ion is selected as one from a group consisting of product ions identified in Table 4, including product ions falling within an identified m/z range of the m/z ratio identified in Table 4 and characterized as having a precursor ion having an m/z ratio within an identified m/z range of the m/z ratio identified in Table 4. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 4, and may be characterized as having a precursor ion having an m/z ratio that falls within at least one of the first range (±0.5), the second range (±1.0), or the third range (±1.0 of the precursor ion m/z ratio identified in Table 4.

In Table 4, “PS-ID No.” identifies the label or index for the peptide structure; “R; “RT (min)” identifies the minimum retention time; “Coll. Energy” identifies the collision energy; “Precur. m/z” identifies the precursor ion m/z ratio; “Precur. Charge” identifies the precursor ion charge; “1^{st} Prod. m/z” identifies the first product ion m/z ratio; “1^{st} Prod. Charge” identifies the first product ion charge; “2^{nd} Prod. m/z” identifies the second product ion m/z ratio; “2^{nd} Prod. Charge” identifies the second product ion charge.

TABLE 4
Peptide Structures and Mass Spectrometry-Related Characteristics

| | | | | | | 2nd | 2nd | | |
|---|---|---|---|---|---|---|---|---|---|
| PS-ID | Monoisotopic | Precursor | Precursor. | Product | Product. | Product | Product | RT | Collision. |
| NO. | mass | m/z | charge | m/z | charge | m/z | charge | (min) | Energy |
| PS-01 | 3959.660292 | 991.2 | 4 | 366.1 | 1 | 980.5 | 2 | 38.2 | 24 |
| PS-02 | 4366.945898 | 1093.2 | 4 | 366.1 | 1 | 1183.6 | 2 | 44 | 22 |
| PS-03 | 2162.173506 | 721.8 | 3 | 371.24 | 1 | 826.48 | 1 | 44.8 | 10 |
| PS-04 | 4686.909744 | 1173.2 | 4 | 366.1 | 1 | 978.5 | 2 | 30.2 | 28 |
| PS-05 | 5436.174062 | 1360.5 | 4 | 366.1 | 1 | 1062.5 | 2 | 37.8 | 25 |
| PS-06 | 3826.597634 | 958.1 | 4 | 274.1 | 1 | 1059 | 2 | 31.6 | 20 |
| PS-07 | 2792.243936 | 931.8 | 3 | 274.1 | 1 | — | — | 37.6 | 22 |
| PS-08 | 2838.255284 | 710.8 | 4 | 274.1 | 1 | 865.5 | 2 | 6.6 | 20 |
| PS-09 | 4714.215082 | 943.9 | 5 | 204.1 | 1 | 1056.2 | 3 | 40.9 | 15 |
| PS-10 | 2957.14418 | 987.1 | 3 | 366.1 | 1 | 1392.6 | 1 | 7.8 | 32 |
| PS-11 | 3054.196942 | 1019.4 | 3 | 204.1 | 1 | 1360.6 | 1 | 13.9 | 32 |
| PS-12 | 2925.154352 | 976.1 | 3 | 366.1 | 1 | — | — | 12.7 | 20 |
| PS-13 | 3655.51799 | 1220.2 | 4 | 366.1 | 1 | 827.9 | 2 | 27 | 35 |
| PS-14 | 1527.73576 | 764.9 | 2 | 234.1 | 1 | 961.5 | 1 | 23.3 | 28 |
| PS-15 | 2015.062182 | 672.8 | 3 | 683.33 | 1 | — | — | 23.65 | 25 |
| PS-16 | 3317.33853 | 1107.1 | 3 | 366.1 | 1 | 804.4 | 2 | 20.6 | 25 |
| PS-17 | 5754.335366 | 1440.3 | 4 | 366.1 | 1 | — | — | 33.6 | 30 |
| PS-18 | 5432.116952 | 1087.7 | 5 | 366.1 | 1 | — | — | 23.8 | 27 |
| PS-19 | 3640.564606 | 911.4 | 4 | 274.1 | 1 | 820.4 | 2 | 35.7 | 20 |
| PS-20 | 3026.237078 | 1009.8 | 3 | 366.1 | 1 | — | — | 16.2 | 25 |
| PS-21 | 3146.265412 | 787.8 | 4 | 274.1 | 1 | — | — | 7.5 | 25 |
| PS-22 | 6822.69942 | 1366.3 | 5 | 366.1 | 1 | 1206.3 | 4 | 36.8 | 30 |
| PS-23 | 4457.884808 | 1116 | 4 | 274.1 | 1 | — | — | 24.6 | 30 |
| PS-24 | 2658.070174 | 887.4 | 3 | 1360.6 | 1 | — | — | 13.1 | 30 |
| PS-25 | 2820.122994 | 941.1 | 3 | 204.1 | 1 | — | — | 13 | 23 |
| PS-26 | 1329.65645 | 444.2 | 3 | 683.3 | 1 | 796.4 | 1 | 13.7 | 11 |
| PS-27 | 3326.461246 | 1109.8 | 3 | 274.1 | 1 | 1190.6 | 2 | 37.6 | 30 |
| PS-28 | 4775.889354 | 1195.3 | 4 | 366.1 | 1 | — | — | 23.4 | 25 |
| PS-29 | 1628.819166 | 543.95 | 3 | 661.37 | 1 | 530.33 | 1 | 37.8 | 15 |

Table 5A defines the peptide sequences for SEQ ID NOS: 1-24 from Table 1A. Table 5A further identifies a corresponding protein SEQ ID NO for each peptide sequence. The corresponding protein SEQ ID NO identifies the protein from which the peptide sequence may be derived.

TABLE 5A
Peptide SEQ ID NOS

| Peptide | | Corresponding |
|---|---|---|
| SEQ | | Protein |
| ID NO. | Peptide.sequence | SEQ ID NO. |
| 1 | YLGNATAIFFLPDEGK | 24 |
| 2 | VSNQTLSLFFTVLQDVPVR | 25 |
| 3 | YTGNASALFILPDQDK | 26 |
| 4 | SVQEIQATFFYFTPNK | 27 and 28 |
| 5 | FEVDSPVYNATWSASLK | 29 |
| 6 | FSEFWDLDPEVRPTSAVAA | 30 |
| 7 | SSTTKPPFKPHGSR | 31 |
| 8 | LSLHRPALEDLLLGSEANLTCTLTGLR | 32 and 33 |
| 9 | EEQFNSTFR | 35 |
| 10 | LQAPLNYTEFQK | 36 |
| 11 | FATTFYQHLADSK | 37 |
| 12 | TVVQPSVGAAAGPVVPPCPGR | 38 |
| 13 | SWPAVGNCSSALR | 39 |
| 14 | QVFPGLNYCTSGAYSNASSTDSASYYPLTGDTR | 29 |
| 15 | QDQCIYNTTYLNVQR | 27 |
| 16 | IYSGILNLSDITK | 36 |
| 17 | VLNFTTK | 40 |
| 18 | GHVNITR | 41 |
| 19 | LNDTLDYECHDGYESNTGSTTGSIVCGYNGWSDLPICY | 42 |
| | ER | |
| 20 | ADGTVNQIEGEATPVNLTEPAK | 43 |
| 21 | INHGIL YDEEK | 44 |
| 22 | DKFSEFWDLDPEVRPTSAVAA | 30 |
| 23 | DSHSLTTNIMEILR | 45 |

Table 5B provides an indication of particular markers and includes the starting position of the peptide sequence within the protein sequence and the end position of the peptide sequence within the protein sequence.

| PS- | | | | |
|---|---|---|---|---|
| ID | | | Start | End |
| NO. | PS-NAME | Peptide.sequence | position | position |
| PS- | A1AT_271_5402 | YLGNATAIFFLPDEGK | 268 | 283 |
| 01 | | | | |
| PS- | A2MG_1424_5402 | VSNQTLSLFFTVLQDVPVR | 1422 | 1440 |
| 02 | | | | |
| PS- | A2MG_1424_NONGLY | VSNQTLSLFFTVLQDVPVR | 1422 | 1440 |
| 03 | COSYLATED | | | |
| PS- | AACT_271_7602 | YTGNASALFILPDQDK | 268 | 283 |
| 04 | | | | |
| PS- | AGP1_93_6503 | QDQCIYNTTYLNVQR | 87 | 101 |
| 28 | | | | |
| PS- | AGP12_72_7604 | SVQEIQATFFYFTPNK | 58 | 73 |
| 05 | | | | |
| PS- | APOB_3895_5401 | FEVDSPVYNATWSASLK | 3887 | 3903 |
| 06 | | | | |
| PS- | APOB_983_5402 | QVFPGLNYCTSGAYSNASSTDSASY | 968 | 1000 |
| 17 | | YPLTGDTR | | |
| PS- | APOC3_74_1101 | FSEFWDLDPEVRPTSAVAA | 81 | 99 |
| 07 | | | | |
| PS- | APOC3_74MC_1102 | DKFSEFWDLDPEVRPTSAVAA | 79 | 99 |
| 27 | | | | |
| PS- | APOD_98_5402 | ADGTVNQIEGEATPVNLTEPAK | 83 | 104 |
| 23 | | | | |
| PS- | AGP1_93_7604 | QDQCIYNTTYLNVQR | 87 | 101 |
| 18 | | | | |
| PS- | CFAH_529_5402 | LNDTLDYECHDGYESNTGSTTGSIV | 528 | 567 |
| 22 | | CGYNGWSDLPICYER | | |
| PS- | CO6_324_5402 | VLNFTTK | 322 | 328 |
| 20 | | | | |
| PS- | KLKB1_453_5402 | IYSGILNLSDITK | 447 | 459 |
| 19 | | | | |
| PS- | THRB_121_5412 | GHVNITR | 118 | 124 |
| 21 | | | | |
| PS- | FETUA_346_NONGLY | TVVQPSVGAAAGPVVPPCPGR | 341 | 361 |
| 15 | COSYLATED | | | |
| PS- | HEMO_187_5401 | SWPAVGNCSSALR | 181 | 193 |
| 16 | | | | |
| PS- | HRG_271_2202 | SSTTKPPFKPHGSR | 271 | 284 |
| 08 | | | | |
| PS- | IGA12_144_4401 | LSLHRPALEDLLLGSEANLTCTLTGL | 127 | 153 |
| 09 | | R | | |
| PS- | IGG1_297_5410 | EEQYNSTYR | 176 | 184 |
| 10 | | | | |
| PS- | IGG2_297_3500 | EEQFNSTFR | 172 | 180 |
| 24 | | | | |
| PS- | IGG2_297_4411 | EEQFNSTFR | 172 | 180 |
| 11 | | | | |
| PS- | IGG2_297_4500 | EEQFNSTFR | 172 | 180 |
| 25 | | | | |
| PS- | IGG2_297_5410 | EEQFNSTFR | 172 | 180 |
| 12 | | | | |
| PS- | KLKB1_494MC_5402 | LQAPLNYTEFQK | 489 | 500 |
| 13 | | | | |
| PS- | PLASMAFGA_DSHSL | DSHSLTTNIMEILR | 101 | 114 |
| 29 | TTNIMEILR | | | |
| PS- | ANT3_FATTFYQHLA | FATTFYQHLADSK | 90 | 102 |
| 14 | DSK | | | |
| PS- | FHR1_INHGILYDEEK | INHGIL YDEEK | 28 | 38 |
| 26 | | | | |

Table 6 identifies the proteins of SEQ TD NOS: 24-45 from Table 5A. Table 6 identifies a corresponding protein abbreviation and protein name for each of protein SEQ TD NOS: 24-45. Further, Table 6 identifies a corresponding Uniprot TD for each of protein SEQ ID NOS: 24-45.

TABLE 6
Protein SEQ ID NOS

| SEQ | Protein | | |
|---|---|---|---|
| ID | Abbre- | | Uniprot |
| NO. | viation | Protein Name | ID |
| 24 | A1AT | Alpha-1 Antitrypsin | P01009 |
| 25 | A2MG | Alpha-2-Macroglobulin | P01023 |
| 26 | AACT | Alpha 1-Antichymotrypsin | P01011 |
| 27 | AGP1 | Alpha-1-acid glycoprotein 1 | P02763 |
| 28 | AGP2 | Alpha-1-acid glycoprotein 2 | P19652 |
| 30 | APOC3 | Apolipoprotein C-III | P02656 |
| 29 | APOB | Apolipoprotein B-100 | P04114 |
| 31 | HRG | Histidine-rich Glycoprotein | P04196 |
| 40 | CO6 | Complement component C6 | P13671 |
| 32 | IGA1 | Immunoglobulin alpha-1 | P01876 |
| 33 | IGA2 | Immunoglobulin alpha-2 | P01877 |
| 34 | IGG1 | Immunoglobulin gamma-1 | P01857 |
| 35 | IGG2 | Immunoglobulin gamma-2 | P01859 |
| 36 | KLKB1 | Plasma Kallikrein | P03952 |
| 37 | ANT3 | Antithrombin-III | P01008 |
| 38 | FETUA | Alpha-2-HS-glycoprotein | P02765 |
| 39 | HEMO | Hemopexin | P02790 |
| 41 | THRB | Prothrombin | P00734 |
| 42 | CFAH | Complement Factor H | P08603 |
| 43 | APOD | Apolipoprotein D | P05090 |
| 44 | FHR1 | Complement factor H-related protein 1 | Q03591 |
| 45 | FGA | Fibrinogen alpha chain | P02671 |

Table 7 identifies and defines the glycan structures included at least in Table 1B. Table 7 identifies a graphical representation of the one or more glycan structures associated with a particular glycan and a coded representation of the composition for each glycan structure included at least in Table 1B. As used herein, the 4-digit GL NO. is a designation that represents the number of hexoses, the number of HexNAcs, the number of Fucoses, and the number of Neuraminic Acids.

TABLE 7
Glycan Structure GL NOS: Structure and Composition

| Composition | Structure | Glycan composition | Class | Glycan Mass |
|---|---|---|---|---|
| 1101 | [symbol] | Hex(1)HexNAc(1)Fuc(0)NeuAc(1) | O-glycan | 656.227598 |
| 1102 | [symbol] | Hex(1)HexNAc(1)Fuc(0)NeuAc(2) | O-glycan | 947.323008 |
| 2202 | [symbol] | Hex(2)HexNAc(2)Fuc(0)NeuAc(2) | O-glycan | 1312.455196 |
| 3500 | [symbol] | Hex(3)HexNAc(5)Fuc(0)NeuAc(0) | N-glycan | 1501.5553 |
| 4401 | [symbol] | Hex(4)HexNAc(4)Fuc(0)NeuAc(1) | N-glycan | 1751.624162 |
| 4411 | [symbol] | Hex(4)HexNAc(4)Fuc(1)NeuAc(0) | N-glycan | 1897.682068 |
| 4500 | [symbol] | Hex(4)HexNAc(5)Fuc(0)NeuAc(0) | N-glycan | 1663.60812 |
| 5400 | [symbol] | Hex(5)HexNAc(4)Fuc(0)NeuAc(0) | N-glycan | 1622.581572 |
| 5401 | [symbol] | Hex(5)HexNAc(4)Fuc(0)NeuAc(1) | N-glycan | 1913.676982 |
| 5402 | [symbol] | Hex(5)HexNAc(4)Fuc(0)NeuAc(2) | N-glycan | 2204.772392 |
| 5410 | [symbol] | Hex(5)HexNAc(4)Fuc(1)NeuAc(0) | N-glycan | 1768.639478 |
| 5411 | [symbol] | Hex(5)HexNAc(4)Fuc(1)NeuAc(1) | N-glycan | 2059.734888 |
| 5412 | [symbol] | Hex(5)HexNAc(4)Fuc(1)NeuAc(2) | N-glycan | 2350.830298 |
| 6311 | [symbol] | Hex(6)HexNAc(3)Fuc(1)NeuAc(1) | N-glycan | 2018.70834 |
| 6503 | [symbol] | Hex(6)HexNAc(5)Fuc(0)NeuAc(3) | N-glycan | 2860.99999 |
| 7602 | [symbol] | Hex(7)HexNAc(6)Fuc(0)NeuAc(2) | N-glycan | 2935.036768 |
| 7604 | [symbol] | Hex(7)HexNAc(6)Fuc(0)NeuAc(4) | N-glycan | 3517.227588 |

Legend for Table 7

| | | | | |
|---|---|---|---|---|
| ● | [symbol] | [symbol] | [symbol] | [symbol] |
| Glc | Gal | Man | Fuc | Neu5Ac |
| ▪ | [symbol] | [symbol] | [symbol] | [symbol] |
| GlcNAc | GlcNAc | ManNAc | Xyl | Neu5Gc |
| [symbol] | [symbol] | [symbol] | | [symbol] |
| GlcN | GalN | ManN | | Kdn |
| [symbol] | [symbol] | [symbol] | [symbol] | |
| GlcA | GalA | ManA | IdoA | |

Aspects of the disclosure include kits comprising one or more compositions, each comprising one or more peptide structures of the disclosure that can be used as assay standards, and instructions for use. Kits in accordance with various embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating various disease states within FLD, including, without limitation, NASH or early/late stage thereof. A transition includes a precursor ion and at least one product ion grouping. As described herein, the peptide structures in Table 1, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, NASH.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system) using, for example, a liquid chromatography system (e.g., a high-performance liquid chromatography system (HPLC)). In various embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a mass spectrometry system (e.g., a reaction monitoring mass spectrometry system) in which one or more product ions may correspond to each of the one or more peptide structures that have been inputted into the mass spectrometry system. As described herein, each peptide structure can be converted into a set of product ions having a defined m/z ratio, as provided in Table 4, or an m/z ratio within an identified range of the m/z ratio provided in Table 4. In some embodiments, the methods involve generating quantification (e.g., abundance) data for the one or more product ions detected using the mass spectrometry system.

In some embodiments, the methods further comprise generating a diagnosis output using the quantification data and a model that has been trained using supervised and/or unsupervised machine learning. In various embodiments, the reaction monitoring mass spectrometry system may include multiple/selected reaction monitoring mass spectrometry (MRM/SRM-MS) to detect the one or more product ions and generate the quantification data. Aspects of the disclosure include compositions comprising one or more of the peptide structures listed in Table 16. In some embodiments, a composition comprises a plurality of the peptide structures listed in Table 16. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, or all of the peptide structures listed in Table 16. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 77-119, listed in Table 16. Aspects of the disclosure include compositions comprising one or more precursor ions having a defined charge and/or defined mass-to-charge (m/z) ratio, as listed in Table 18. Aspects of the disclosure include compositions comprising one or more product ions having a defined mass-to-charge (m/z) ratio, which product ions are produced by converting a peptide structure described herein (e.g., a peptide structure listed in Table 16) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 16). In some embodiments, a composition comprises a set of the product ions listed in Table 18, having an m/z ratio selected from the list provided for each peptide structure in Table 16 or Table 18.

In some embodiments, a composition comprises at least one of peptide structures PS-48 through PS-102 identified in Table 16. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, or all 55 of the peptide structures PS-48 through PS-102 in Table 16.

In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures PS-49, PS-50, PS-54, PS-61, PS-63, PS-64, PS-71, PS-79, PS-81, PS-84, PS-86, PS-87, PS-90, PS-91, PS-92, PS-94, PS-95, PS-96, PS-97, PS-98, PS-99, or PS-101 in Table 17A. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or all 19 of the peptide structures PS-48, PS-52, PS-57, PS-61, PS-62, PS-63, PS-64, PS-69, PS-71, PS-72, PS-73, PS-84, PS-86, PS-88, PS-91, PS-94, PS-96, PS-100, or PS-101 in Table 17B.

In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, or all 17 of the peptide structures PS-48, PS-52, PS-61, PS-64, PS-66, PS-68, PS-69, PS-71, PS-72, PS-73, PS-86, PS-89, PS-91, PS-94, PS-96, PS-99, or PS-101 in Table 17C.

In some embodiments, a composition comprises a peptide structure or a product ion. The peptide structure or product ion comprises an amino acid sequence having at least 90% sequence identity to any one of SEQ ID NOS: 77-119, as identified in Table 19, corresponding to peptide structures PS-48 through PS-102 in Table 16.

In some embodiments, a composition comprises a peptide structure or a product ion. The peptide structure or product ion comprises an amino acid sequence having at least 90% sequence identity to any one of SEQ ID NOS: 77-119, as identified in Table 19, corresponding to peptide structures PS-48 through PS-102 in Table 16.

In some embodiments, the product ion is selected as one from a group consisting of product ions identified in Table 18, including product ions falling within an identified m/z range of the m/z ratio identified in Table 18 and characterized as having a precursor ion having an m/z ratio within an identified m/z range of the m/z ratio identified in Table 18. A first range for the product ion m/z ratio may be ±0.5. A second range for the product ion m/z ratio may be ±0.8. A third range for the product ion m/z ratio may be 1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (1.0) of the product ion m/z ratio identified in Table 18, and characterized as having a precursor ion having an m/z ratio that falls within at least one of first range (±0.5), a second range (1.0), or a third range (1.0 of the precursor ion m/z ratio identified in Table 18.

TABLE 18
Mass Spectrometry-Related Characteristics for the Peptide
Structures associated with Pancreatic Cancer

| | Mono- | | | | | | 2nd |
|---|---|---|---|---|---|---|---|
| PS- | isotopic | RT | Precursor | Precurs. | | | Product |
| NAME | mass | (min) | m/z | charge | Product m/z | Collision.Energy | m/z |
| 48 | 6041.646782 | 42.8 | 1209.8 | 5 | 366.1 | 30 | 1299 |
| 49 | 6406.77897 | 42.7 | 1282.9 | 5 | 366.1 | 30 | 1299 |
| 50 | 3690.816484 | 42.5 | 924.3 | 4 | 833.9 | 25 | 782.3 |
| 51 | 5385.40009 | 47.1 | 1347.9 | 4 | 366.1 | 35 | 274.1 |
| 52 | 6040.44195 | 33.3 | 1209.7 | 5 | 1347.9 | 15 | 366.1 |
| 53 | 2162.173506 | 44.8 | 721.8 | 3 | 371.24 | 10 | 826.48 |
| 54 | 4950.310912 | 38.8 | 1239.1 | 4 | 1314.2 | 25 | N/A |
| 55 | 5647.565058 | 39.3 | 1413.6 | 4 | 366.1 | 30 | 1313.3 |
| 56 | 4344.878414 | 40.165 | 1450 | 3 | 366.1 | 30 | 1318.7 |
| 57 | 4747.055968 | 42 | 1188.2 | 4 | 366.1 | 25 | N/A |
| 58 | 5326.297412 | 35.1 | 1066.7 | 5 | 366.1 | 22 | 1206.6 |
| 59 | 5617.392822 | 35.9 | 1124.9 | 5 | 366.1 | 25 | 1206.2 |
| 60 | 5285.270864 | 35.2 | 1058.5 | 5 | 366.1 | 20 | 1206.6 |
| 61 | 4467.835462 | 30.6 | 1118.2 | 4 | 366.1 | 30 | 978.5 |
| 62 | 5578.174858 | 23.8 | 1116.9 | 5 | 366.1 | 25 | 1060 |
| 63 | 3146.170176 | 5 | 1050.1 | 3 | 274.1 | 35 | 999.4 |
| 64 | 4562.887832 | 35.9 | 1142.2 | 4 | 366.1 | 20 | N/A |
| 65 | 5755.448988 | 41 | 1152.7 | 5 | 366.1 | 28 | 1550.3 |
| 66 | 3826.597634 | 31.6 | 958.1 | 4 | 274.1 | 20 | 1059 |
| 67 | 5754.335366 | 33.6 | 1440.3 | 4 | 366.1 | 30 | N/A |
| 68 | 3600.388632 | 19.1 | 1201.5 | 3 | 274.1 | 30 | 1453.6 |
| 69 | 4444.818994 | 29 | 1112.7 | 4 | 366.1 | 20 | 1368.1 |
| 70 | 4735.914404 | 29.8 | 1185.5 | 4 | 366.1 | 25 | 1368.1 |
| 71 | 5730.401612 | 41 | 1147.8 | 5 | 366.1 | 25 | 366.1 |
| 72 | 4476.821874 | 27.4 | 1120.7 | 4 | 204.1 | 25 | 1165.5 |
| 73 | 2582.037538 | 3.9 | 862 | 3 | 366.1 | 20 | N/A |
| 74 | 2873.132948 | 3.8 | 959.1 | 3 | 366.1 | 30 | N/A |
| 75 | 4121.669544 | 27.1 | 1031.9 | 4 | 204.1 | 30 | N/A |
| 76 | 3754.491846 | 21.3 | 1253.2 | 3 | 366.1 | 30 | 804.3 |
| 77 | 3648.549244 | 29.7 | 913.4 | 4 | 366.1 | 15 | 1234 |
| 78 | 4731.839512 | 40.5 | 1184.5 | 4 | 204.1 | 35 | N/A |
| 79 | 7034.688732 | 13.2 | 1173.6 | 6 | 366.1 | 29 | N/A |
| 80 | 4218.740064 | 28.4 | 1056.2 | 4 | 366.1 | 25 | 366.1 |
| 81 | 3259.26547 | 10 | 1087.8 | 3 | 366.1 | 30 | 1112.5 |
| 82 | 4304.857476 | 35.5 | 1077.7 | 4 | 204.1 | 30 | 1152.6 |
| 83 | 4450.915382 | 35.4 | 1114.2 | 4 | 204.1 | 30 | 1152.6 |
| 84 | 4961.085074 | 35.8 | 1241.8 | 4 | 204.1 | 35 | 1152.6 |
| 85 | 5107.14298 | 35.7 | 1278.3 | 4 | 204.1 | 40 | 1152.6 |
| 86 | 4517.13034 | 39.4 | 1130.8 | 4 | 204.1 | 35 | 1258.7 |
| 87 | 4464.14622 | 40.2 | 1117.1 | 4 | 204.1 | 27 | N/A |
| 88 | 2633.03854 | 7.9 | 879 | 3 | 204.1 | 21 | 1392.6 |
| 89 | 2690.060002 | 8.1 | 898 | 3 | 1392.6 | 25 | N/A |
| 90 | 2836.117908 | 8.1 | 946.5 | 3 | 204.1 | 15 | 1392.6 |
| 91 | 2658.070174 | 13.1 | 887.4 | 3 | 1360.6 | 30 | N/A |
| 92 | 4228.73181 | 31 | 1058.3 | 4 | 1284.7 | 20 | N/A |
| 93 | 5107.176866 | 33.5 | 1277.8 | 4 | 366.1 | 38 | N/A |
| 94 | 1178.665896 | 30.6 | 590.3 | 2 | 725.4 | 15 | 342.2 |
| 95 | 1234.680878 | 35.2 | 618.3 | 2 | 736.4 | 17 | 936.5 |
| 96 | 816.485756 | 23.2 | 409.2 | 2 | 599.4 | 10 | 486.3 |
| 97 | 1121.61928 | 9.5 | 561.8 | 2 | 244.2 | 25 | 455.3 |
| 98 | 1542.756554 | 22.7 | 772.4 | 2 | 680.3 | 22 | 978.4 |
| 99 | 2454.143774 | 33.7 | 819.1 | 3 | 855.5 | 25 | 609.3 |
| 100 | 3247.313088 | 16.9 | 1083.8 | 3 | 204.1 | 30 | N/A |
| 101 | 3389.421198 | 26.2 | 1131.1 | 3 | 366.1 | 28 | 840.4 |
| 102 | 2824.143062 | 23.8 | 942.4 | 3 | 366.1 | 23 | 1114.6 |

Table 19A defines the peptide sequences for SEQ ID NOS: 77-119 from Table 16. Table 19 further identifies a corresponding protein SEQ TD NO. for each peptide sequence.

TABLE 19A
Peptide SEQ ID NOS

| Peptide | | Corresponding |
|---|---|---|
| SEQ ID | | Protein |
| NO. | Peptide | SEQ ID NO. |
| 77 | ADTHDEILEGLNFNLTEIPEAQIHEGFQELLR | 120 |
| 78 | QLAHQSNSTNIFFSPVSIATAFAMLSLGTK | 120 |
| 79 | EGDHEFLEVPEAQEDVEATFPVHQPGNYSCSY | 121 |
| | R | |
| 80 | VSNQTLSLFFTVLQDVPVR | 122 |
| 81 | IITILEEEMNVSVCGLYTYGKPVPGHVTVSICR | 122 |
| 82 | IITILEEEMNVSVCGLYTYGK | 122 |
| 83 | GCVLLSYLNETVTVSASLESVR | 122 |
| 84 | SLGNVNFTVSAEALESQELCGTEVPSVPEHGR | 122 |
| 85 | YTGNASALFILPDQDK | 123 |
| 86 | QDQCIYNTTYLNVQR | 123 |
| 87 | NEEYNK | 125 & 126 |
| 88 | SVQEIQATFFYFTPNK | 125 & 126 |
| 89 | SVQEIQATFFYFTPNKTEDTIFLR | 125 & 126 |
| 90 | FEVDSPVYNATWSASLK | 127 |
| 91 | QVFPGLNYCTSGAYSNASSTDSASYYPLTGDTR | 127 |
| 92 | LGNWSAMPSCK | 128 |
| 93 | TELFSSSCPGGIMLNETGQGYQR | 129 |
| 94 | NCGVNCSGDVFTALIGEIASPNYPKPYPENSR | 130 |
| 95 | ENLTAPGSDSAVFFEQGTTR | 131 |
| 96 | ANISHK | 132 |
| 97 | VCQDCPLLAPLNDTR | 133 |
| 98 | SWPAVGNCSSALR | 134 |
| 99 | ALPQPQNVTSLLGCTH | 134 |
| 100 | CSDGWSFDATTLDDNGTMLFFK | 134 |
| 101 | NLFLNHSENATAK | 135 |
| 102 | VIDFNCTTSSVSSALANTK | 136 |
| 103 | DTFVNASR | 137 |
| 104 | VLSNNSDANLELINTWVAK | 137 |
| 105 | VGQLQLSHNLSL VIL VPQNLK | 137 |
| 106 | LSLHRPALEDLLLGSEANLTCTLTGLR | 138 & 139 |
| 107 | EEQYNSTYR | 140 |
| 108 | EEQFNSTFR | 141 |
| 109 | STGKPTLYNVSLVMSDTAGTCY | 142 |
| 110 | LQAPLNYTEFQKPICLPSK | 143 |
| 111 | DLLLPQPDLR | 144 |
| 112 | DLATVYVDVLK | 145 |
| 113 | AFLLTPR | 146 |
| 114 | VNHVTLSQPK | 147 |
| 115 | SYTITGLQPGTDYK | 148 |
| 116 | TSESGELHGLTTEEEFVEGIYK | 149 |
| 117 | LDVDQALNR | 150 |
| 118 | CGLVPVLAENYNK | 151 |
| 119 | NGSLFAFR | 152 |

Table 19B provides an indication of particular markers and includes the starting position of the peptide sequence within the protein sequence and the end position of the peptide sequence within the protein sequence.

TABLE 19B
Markers and Protein Positions

| | | | Start | End |
|---|---|---|---|---|
| PS-ID | PS-NAME | Peptide sequence | position | position |
| 48 | A1AT_107_5412 | ADTHDEILEGLNFNLTEIPEAQI | 94 | 125 |
| | | HEGFQELLR | | |
| 49 | A1AT_107_6512 | ADTHDEILEGLNFNLTEIPEAQI | 94 | 125 |
| | | HEGFQELLR | | |
| 50 | A1AT_107_ | ADTHDEILEGLNFNLTEIPEAQI | 94 | 125 |
| | NONGLYCOSYLATED | HEGFQELLR | | |
| 51 | A1AT_70_5402 | QLAHQSNSTNIFFSPVSIATAFA | 64 | 93 |
| | | MLSLGTK | | |
| 52 | A1BG_179_5402 | EGDHEFLEVPEAQEDVEATFP | 153 | 185 |
| | | VHQPGNYSCSYR | | |
| 53 | A2MG_1424_ | VSNQTLSLFFTVLQDVPVR | 1422 | 1440 |
| | NONGLYCOSYLATED | | | |
| 54 | A2MG_247_5200 | IITILEEEMNVSVCGLYTYGKP | 238 | 270 |
| | | VPGHVTVSICR | | |
| 55 | A2MG_247_5401 | IITILEEEMNVSVCGLYTYGKP | 238 | 270 |
| | | VPGHVTVSICR | | |
| 56 | A2MG_247MC_5401 | IITILEEEMNVSVCGLYTYGK | 238 | 258 |
| 57 | A2MG_55_5412 | GCVLLSYLNETVTVSASLESVR | 47 | 68 |
| 58 | A2MG_869_5401 | SLGNVNFTVSAEALESQELCG | 864 | 895 |
| | | TEVPSVPEHGR | | |
| 59 | A2MG_869_5402 | SLGNVNFTVSAEALESQELCG | 864 | 895 |
| | | TEVPSVPEHGR | | |
| 60 | A2MG_869_6301 | SLGNVNFTVSAEALESQELCG | 864 | 895 |
| | | TEVPSVPEHGR | | |
| 61 | AACT_271_6512 | YTGNASALFILPDQDK | 268 | 283 |
| 62 | AGP1_93_7614 | QDQCIYNTTYLNVQR | 87 | 101 |
| 63 | AGP12_56_5412 | NEEYNK | 52 | 57 |
| 64 | AGP12_72_7601 | SVQEIQATFFYFTPNK | 58 | 73 |
| 65 | AGP12_72MC_6503 | SVQEIQATFFYFTPNKTEDTIFL | 58 | 81 |
| | | R | | |
| 66 | APOB_3895_5401 | FEVDSPVYNATWSASLK | 3887 | 3903 |
| 67 | APOB_983_5402 | QVFPGLNYCTSGAYSNASSTD | 968 | 1000 |
| | | SASYYPLTGDTR | | |
| 68 | APOH_253_5412 | LGNWSAMPSCK | 251 | 261 |
| 69 | APOM_135_5401 | TELFSSSCPGGIMLNETGQGYQ | 121 | 143 |
| | | R | | |
| 70 | APOM_135_5402 | TELFSSSCPGGIMLNETGQGYQ | 121 | 143 |
| | | R | | |
| 71 | C1S_174_5402 | NCGVNCSGDVFTALIGEIASPN | 170 | 201 |
| | | YPKPYPENSR | | |
| 72 | CERU_397_5412 | ENLTAPGSDSAVFFEQGTTR | 396 | 415 |
| 73 | CO5_741_5401 | ANISHK | 740 | 745 |
| 74 | CO5_741_5402 | ANISHK | 740 | 745 |
| 75 | FETUA_156_5412 | VCQDCPLLAPLNDTR | 145 | 159 |
| 76 | HEMO_187_5412 | SWPAVGNCSSALR | 181 | 193 |
| 77 | HEMO_453_5401 | ALPQPQNVTSLLGCTH | 447 | 462 |
| 78 | HEMO_64_5402 | CSDGWSFDATTLDDNGTMLFF | 50 | 71 |
| | | K | | |
| 79 | HPT_207_121015 | NLFLNHSENATAK | 203 | 215 |
| 80 | HRG_125_5402 | VIDFNCTTSSVSSALANTK | 121 | 139 |
| 81 | IC1_238_5412 | DTFVNASR | 234 | 241 |
| 82 | IC1_253_5402 | VLSNNSDANLELINTWVAK | 250 | 268 |
| 83 | IC1_253_5412 | VLSNNSDANLELINTWVAK | 250 | 268 |
| 84 | IC1_253_6503 | VLSNNSDANLELINTWVAK | 250 | 268 |
| 85 | IC1_253_6513 | VLSNNSDANLELINTWVAK | 250 | 268 |
| 86 | IC1_352_5402 | VGQLQLSHNLSLVILVPQNLK | 344 | 364 |
| 87 | IGA12_144_3500 | LSLHRPALEDLLLGSEANLTCT | 127 | 153 |
| | | LTGLR | (P01876) or | (P01876) or |
| | | | 114 | 140 |
| | | | (P01877) | (P01877) |
| 88 | IGG1_297_3410 | EEQYNSTYR | 176 | 184 |
| 89 | IGG1_297_3500 | EEQYNSTYR | 176 | 184 |
| 90 | IGG1_297_3510 | EEQYNSTYR | 176 | 184 |
| 91 | IGG2_297_3500 | EEQFNSTFR | 172 | 180 |
| 92 | IGM_439_9200 | STGKPTLYNVSLVMSDTAGTC | 432 | 453 |
| | | Y | | |
| 93 | KLKB1_494_6503 | LQAPLNYTEFQKPICLPSK | 489 | 507 |
| 94 | QUANTPEP- | DLLLPQPDLR | 230 | 239 |
| | A2GL_DLLLPQPDLR | | | |
| 95 | QUANTPEP- | DLATVYVDVLK | 37 | 47 |
| | APOA1_ | | | |
| | DLATVYVDVLK | | | |
| 96 | QUANTPEP- | AFLLTPR | 172 | 178 |
| | APOM_AFLLTPR | | | |
| 97 | QUANTPEP- | VNHVTLSQPK | 102 | 111 |
| | B2M_VNHVTLSQPK | | | |
| 98 | QUANTPEP- | SYTITGLQPGTDYK | 1958 | 1971 |
| | FINC_ | | | |
| | SYTITGLQPGTDYK | | | |
| 99 | QUANTPEP- | TSESGELHGLTTEEEFVEGIYK | 69 | 90 |
| | TTR_ | | | |
| | TSESGELHGLTTEEE | | | |
| | FVEGIYK | | | |
| 100 | SHBG_380_5402 | LDVDQALNR | 373 | 381 |
| 101 | TRFE_432_5401 | CGLVPVLAENYNK | 421 | 433 |
| 102 | VTNC_169_5401 | NGSLFAFR | 169 | 176 |

Table 20 identifies the proteins of SEQ ID NOS: 44-76 from Table 16. Table 20 identifies a corresponding protein abbreviation and protein name for each of protein SEQ ID NOS: 44-76. Further, Table 20 identifies a corresponding Uniprot ID for each of protein SEQ ID NOS: 44-76.

TABLE 20
Protein SEQ ID NOS

| Protein | | (Protein) | |
|---|---|---|---|
| Abbre- | | SEQ ID | |
| viation | Protein Name | NO. | Uniprot ID |
| A1AT | Alpha-1-antitrypsin | 120 | P01009 |
| A1BG | Alpha-1B-glycoprotein | 121 | P04217 |
| A2MG | Alpha-2-macroglobulin | 122 | P01023 |
| AACT | Alpha-1-antichymotrypsin | 123 | P01011 |
| AGP1 | Alpha-1-acid glycoprotein 1 | 124 | P02763 |
| AGP12 | Alpha-1-acid glycoprotein 1&2 | 125 & | P02763&P19652 |
| | | 126 | |
| APOB | Apolipoprotein B-100 | 127 | P04114 |
| APOH | Beta-2-glycoprotein1 | 128 | P02749 |
| APOM | Apolipoprotein M | 129 | O95445 |
| C1S | Complement Cls subcomponent | 130 | P09871 |
| CERU | Ceruloplasmin | 131 | P00450 |
| CO5 | ComplementC5 | 132 | P01031 |
| FETUA | Alpha-2-HS-glycoprotein | 133 | P02765 |
| HEMO | Hemopexin | 134 | P02790 |
| HPT | Haptoglobin | 135 | P00738 |
| HRG | Histidine-rich Glycoprotein | 136 | P04196 |
| IC1 | Plasma protease C1 inhibitor | 137 | P05155 |
| IGA12 | Immunoglobulin heavy | 138 & | P01876 or |
| | constant alpha 1&2 | 139 | P01877 |
| IGG1 | Immunoglobulin heavy | 140 | P01857 |
| | constant gamma 1 | | |
| IGG2 | Immunoglobulin heavy | 141 | P01859 |
| | constant gamma 2 | | |
| IGM | Immunoglobulin heavy | 142 | P01871 |
| | constant mu | | |
| KLKB1 | Plasma Kallikrein | 143 | P03952 |
| A2GL | Leucine-richAlpha- | 144 | P02750 |
| | 2-glycoprotein | | |
| APOA1 | Apolipoprotein A-I | 145 | P02647 |
| APOM | Apolipoprotein M | 146 | O95445 |
| B2M | Beta-2-microglobulin | 147 | P61769 |
| FINC | Fibronectin | 148 | P02751 |
| TTR | Transthyretin | 149 | P02766 |
| SHBG | Sex hormone-binding globulin | 150 | P04278 |
| TRFE | Serotransferrin | 151 | P02787 |
| VTNC | Vitronectin | 152 | P04004 |

Table 21 identifies and defines the glycan structures included in Table 16, all of which are N-glycans. Table 21 identifies a coded representation of the composition for each glycan structure included in Table 16. As used herein, the 4-digit GL NO. is a designation that represents the number of hexoses, the number of HexNAcs, the number of Fucoses, and the number of Neuraminic Acids.

TABLE 21
Glycan Structure GL NOS: Composition

| Glycan Structure | | | |
|---|---|---|---|
| GL NO. | Structure | Glycan Composition | Mass |
| 5412 | [symbol] | Hex(5)HexNAc(4)Fuc(1)NeuAc(2) | 2368.840798 |
| 6512 | [symbol] | Hex(6)HexNAc(5)Fuc(1)NeuAc(1) | 2733.972986 |
| 5402 | [symbol] | Hex(5)HexNAc(4)Fuc(0)NeuAc(2) | 2222.782892 |
| 5200 | [symbol] | Hex(5)HexNAc(2)Fuc(0)NeuAc(0) | 1234.433336 |
| 5401 | [symbol] | Hex(5)HexNAc(4)Fuc(0)NeuAc(1) | 1931.687482 |
| 6301 | [symbol] | Hex(6)HexNAc(3)Fuc(0)NeuAc(1) | 1890.660934 |
| 6512 | [symbol] | Hex(6)HexNAc(5)Fuc(1)NeuAc(1) | 2733.972986 |
| 7614 | [symbol] | Hex(7)HexNAc(6)Fuc(1)NeuAc(4) | 3681.295994 |
| 7601 | [symbol] | Hex(7)HexNAc(6)Fuc(0)NeuAc(1) | 2661.951858 |
| 6503 | [symbol] | Hex(6)HexNAc(5)Fuc(0)NeuAc(3) | 2879.01049 |
| 6502 | [symbol] | Hex(6)HexNAc(5)Fuc(0)NeuAc(2) | 2587.91508 |
| 6513 | [symbol] | Hex(6)HexNAc(5)Fuc(1)NeuAc(3) | 3025.068396 |
| 3500 | [symbol] | Hex(3)HexNAc(5)Fuc(0)NeuAc(0) | 1519.5658 |
| 3410 | [symbol] | Hex(3)HexNAc(4)Fuc(1)NeuAc(0) | 1462.544338 |
| 3510 | [symbol] | Hex(3)HexNAc(5)Fuc(1)NeuAc(0) | 1665.623706 |
| 9200 | [symbol] | Hex(9)HexNAc(2)Fuc(0)NeuAc(0) | 1882.644616 |

Legend for Table 21:

| | | | | |
|---|---|---|---|---|
| ● | [symbol] | [symbol] | [symbol] | [symbol] |
| Glc | Gal | Man | Fuc | Neu5Ac |
| ▪ | [symbol] | [symbol] | [symbol] | [symbol] |
| GlcNAc | GlcNAc | ManNAc | Xyl | Neu5Gc |
| [symbol] | [symbol] | [symbol] | | [symbol] |
| GlcN | GalN | ManN | | Kdn |
| [symbol] | [symbol] | [symbol] | [symbol] | |
| GlcA | GalA | ManA | IdoA | |

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating a PC disease state. A transition includes a precursor ion and at least one product ion grouping. As reviewed herein, the peptide structures in Table 16, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, PC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 18 or an m/z ratio within an identified m/z ratio as provided in Table 18. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VIII.A. Compositions and Kits—NSCLC

Provided herein are compositions comprising one or more peptide structures from Table 35. Provided herein are compositions comprising one or more glycopeptides from Table 35. In some embodiments, provided herein is a composition comprising two or more peptide structures from Table 35. In some embodiments, provided herein is a composition comprising three or more peptide structures from Table 35. In some embodiments, provided herein is a composition comprising four or more peptide structures from Table 35. In some embodiments, provided herein is a composition comprising five or more peptide structures from Table 35. In some embodiments, provided herein is a composition comprising 10 or more peptide structures from Table 35. In some embodiments, provided herein is a composition comprising 15 or more peptide structures from Table 35. In some embodiments, provided herein is a composition comprising 20 or more peptide structures from Table 35. In some embodiments, provided herein is a composition comprising 25 or more peptide structures from Table 35. In some embodiments, the composition is from a biological sample. In some embodiments, the composition comprises one or more purified peptide structures. In some embodiments, the composition comprises one or more purified glycopeptides. In some embodiments, the composition comprises enzymatically digested peptide and/or glycopeptide fragments, such as those in Table 35. In some embodiments, the composition comprises enzymatically digested glycopeptide fragments, such as those in Table 35. In some embodiments, the composition comprises at least one, at least two, at least three, at least four, at least five, at least 10, at least 15, at least 20, or at least 25 peptides and/or glycopeptides comprising a sequence set forth in SEQ ID NOs: 224-296 along with the associated glycan set forth in Table 35.

In some embodiments, provided herein is a composition comprising one or more peptide structures from Table 40. In some embodiments, provided herein is a composition comprising one or more glycopeptides from Table 40. In some embodiments, provided herein is a composition comprising two or more peptide structures from Table 40. In some embodiments, provided herein is a composition comprising three or more peptide structures from Table 40. In some embodiments, provided herein is a composition comprising four or more peptide structures from Table 40. In some embodiments, provided herein is a composition comprising five or more peptide structures from Table 40. In some embodiments, provided herein is a composition comprising 10 or more peptide structures from Table 40. In some embodiments, provided herein is a composition comprising 15 or more peptide structures from Table 40. In some embodiments, the composition is from a biological sample. In some embodiments, the composition comprises one or more purified peptide structures. In some embodiments, the composition comprises one or more purified glycopeptides. In some embodiments, the composition comprises enzymatically digested peptide fragments, such as those in Table 40. In some embodiments, the composition comprises enzymatically digested glycopeptide fragments, such as those in Table 40. In some embodiments, the composition comprises at least one, at least two, at least three, at least four, at least five, at least 10, or at least 15 peptides and/or glycopeptides comprising a sequence set forth in SEQ ID NOs: 229, 231-234, 236, 239, 241, 244-245, 247-255 along with the associated glycan set forth in Table 35.

In some embodiments, provided herein is a composition comprising at least one peptide and/or glycopeptide comprising a sequence set forth in SEQ ID NOs: 224-296 along with the associated glycan set forth in Table 35. In some embodiments, provided herein is a composition comprising at least two peptides and/or glycopeptides comprising a sequence set forth in SEQ ID NOs: 224-296 along with the associated glycan set forth in Table 35. In some embodiments, provided herein is a composition comprising at least three peptides and/or glycopeptides comprising a sequence set forth in SEQ ID NOs: 224-296 along with the associated glycan set forth in Table 35. In some embodiments, provided herein is a composition comprising at least four peptides and/or glycopeptides comprising a sequence set forth in SEQ ID NOs: 224-296 along with the associated glycan set forth in Table 35. In some embodiments, provided herein is a composition comprising at least five peptides and/or glycopeptides comprising a sequence set forth in SEQ ID NOs: 224-296 along with the associated glycan set forth in Table 35. In some embodiments, provided herein is a composition comprising at least 10 peptides and/or glycopeptides comprising a sequence set forth in SEQ ID NOs: 224-296 along with the associated glycan set forth in Table 35. In some embodiments, provided herein is a composition comprising at least 15 peptides and/or glycopeptides comprising a sequence set forth in SEQ ID NOs: 224-296 along with the associated glycan set forth in Table 35. In some embodiments, provided herein is a composition comprising 20 peptides and/or glycopeptides comprising sequences set forth in SEQ ID NOs: 224-296 along with the associated glycan set forth in Table 35. In some embodiments, provided herein is a composition comprising 25 peptides and/or glycopeptides comprising sequences set forth in SEQ ID NOs: 224-296 along with the associated glycan set forth in Table 35.

In some embodiments, provided herein are peptides and/or glycopeptides set forth in Table 35. In some embodiments, provided herein are glycopeptides set forth in Table 35. In some embodiments, provided herein are peptides and/or glycopeptides comprising a sequence set forth in SEQ ID NOs: 224-296 along with the associated glycan set forth in Table 35.

In some embodiments, provided herein is a composition comprising at least one peptide and/or glycopeptides comprising a sequence set forth in SEQ ID NOs: 229, 231-234, 236, 239, 241, 244-245, 247-255 along with the associated glycan set forth in Table 35. In some embodiments, provided herein is a composition comprising at least two peptides and/or glycopeptides comprising a sequence set forth in SEQ ID NOs: 229, 231-234, 236, 239, 241, 244-245, 247-255 along with the associated glycan set forth in Table 35. In some embodiments, provided herein is a composition comprising at least three peptides and/or glycopeptides comprising a sequence set forth in SEQ ID NOs: 229, 231-234, 236, 239, 241, 244-245, 247-255 along with the associated glycan set forth in Table 35. In some embodiments, provided herein is a composition comprising at least four peptides and/or glycopeptides comprising a sequence set forth in SEQ ID NOs: 229, 231-234, 236, 239, 241, 244-245, 247-255 along with the associated glycan set forth in Table 35. In some embodiments, provided herein is a composition comprising at least five peptides and/or glycopeptides comprising a sequence set forth in SEQ ID NOs: 229, 231-234, 236, 239, 241, 244-245, 247-255 along with the associated glycan set forth in Table 35. In some embodiments, provided herein is a composition comprising at least 10 peptides and/or glycopeptides comprising a sequence set forth in SEQ ID NOs: 229, 231-234, 236, 239, 241, 244-245, 247-255 along with the associated glycan set forth in Table 35. In some embodiments, provided herein is a composition comprising at least 15 peptides and/or glycopeptides comprising a sequence set forth in SEQ ID NOs: 229, 231-234, 236, 239, 241, 244-245, 247-255 along with the associated glycan set forth in Table 35.

In some embodiments, provided herein are peptides and/or glycopeptides set forth in Table 40. In some embodiments, provided herein are glycopeptides set forth in Table 40. In some embodiments, provided herein are peptides and/or glycopeptides comprising a sequence set forth in SEQ ID NOs: 229, 231-234, 236, 239, 241, 244-245, 247-255 along with the associated glycan set forth in Table 35.

Also provided herein are kits for detecting NSCLC in an individual and kits for treating NSCLC in an individual. In some embodiments, the kit comprises at least one agent for quantifying at least one peptide structure identified in Table 35. In some embodiments, the kit comprises at least one agent for quantifying at least one peptide structure comprising the amino acid sequence of SEQ ID NOs: 224-296 along with the associated glycan set forth in Table 35. In some embodiments, the kit comprises at least one agent for quantifying at least one peptide structure identified in Table 40. In some embodiments, the kit comprises at least one agent for quantifying at least one peptide structure comprising the amino acid sequence of SEQ ID NOs: 229, 231-234, 236,239, 241, 244-245,247-255 along with the associated glycan set forth in Table 35. In some embodiments, the kit comprises at least one agent for quantifying at least one peptide structure, wherein the agent is from a biological sample, is one or more purified peptide structures, and/or comprises enzymatically digested peptide and/or glycopeptide fragments. In some embodiments, the kit comprises instructions for detecting one or more biomarkers provided herein, wherein the biomarker is at least one peptide structure identified in Table 35. In some embodiments, the kit comprises instructions for detecting one or more biomarkers provided herein, wherein the biomarker is at least one glycopeptide identified in Table 35. In some embodiments, the kit comprises instructions for detecting one or more biomarkers provided herein, wherein the biomarker is at least one peptide structure comprising the amino acid sequence of SEQ ID NOs: 224-296 along with the associated glycan set forth in Table 35. In some embodiments, the kit comprises instructions for detecting one or more biomarkers provided herein, wherein the biomarker is at least one peptide structure identified in Table 40. In some embodiments, the kit comprises instructions for detecting one or more biomarkers provided herein, wherein the biomarker is at least one peptide structure comprising the amino acid sequence of SEQ ID NOs: 229, 231-234, 236, 239, 241, 244-245, 247-255 along with the associated glycan set forth in Table 35. In some embodiments, the kit comprises reagents for detecting one or more biomarkers described herein. In some embodiments, the kit comprises at least one agent for quantifying at least one peptide structure identified in Table 35 to carry out part or all of any one or more of the methods disclosed herein. In some embodiments, the kit comprises at least one agent for quantifying at least one peptide structure comprising the amino acid sequence of SEQ ID NOs: 224-296 along with the associated glycan set forth in Table 35 to carry out part or all of any one or more of the methods disclosed herein. In some embodiments, the kit comprises at least one agent for quantifying at least one peptide structure identified in Table 40 to carry out part or all of any one or more of the methods disclosed herein. In some embodiments, the kit comprises at least one agent for quantifying at least one peptide structure comprising the amino acid sequence of SEQ ID NOs: 229, 231-234, 236,239, 241, 244-245,247-255 along with the associated glycan set forth in Table 35 to carry out part or all of any one or more of the methods disclosed herein.

In some embodiments, the kit comprises at least one of a peptide standard, a glycopeptide standard, a buffer, and at least one or more peptide sequences to carry out part or all of any one or more of the methods disclosed herein. In some embodiments, the kit comprises at least one of a peptide standard, a glycopeptide standard, a buffer, and at least one or more glycopeptide sequences to carry out part or all of any one or more of the methods disclosed herein. In some embodiments, the one or more peptide and/or glycopeptide sequences are identified in Table 35. In some embodiments, the one or more peptide and/or glycopeptide sequences comprise the amino acid sequence of SEQ ID NOs: 224-296 along with the associated glycan set forth in Table 35. In some embodiments, the one or more peptide and/or glycopeptide sequences are identified in Table 40. In some embodiments, the one or more peptide and/or glycopeptide sequences comprise the amino acid sequence of SEQ ID NOs: 229, 231-234, 236, 239, 241, 244-245, 247-255 along with the associated glycan set forth in Table 35. In some embodiments, the kit comprises instructions for detecting NSCLC in an individual. In some embodiments, the kit comprises instructions for diagnosing NSCLC in an individual. In some embodiments, the kit comprises instructions for use according to any of the methods provided herein.

In some embodiments, the kit comprises instructions for selecting a treatment for the individual having NSCLC. In some embodiments, the kit comprises instructions for administering a treatment to the individual having NSCLC.

In some embodiments, the kit comprises reagents and instructions for selecting a treatment for an individual with NSCLC, wherein the treatment is one or more NSCLC treatments described herein. In some embodiments, the kit comprises reagents and instructions for administering the treatment to an individual with NSCLC comprising administering to the individual one or more NSCLC treatments described herein. In some embodiments, the kit comprises reagents and instructions for treating an individual diagnosed with NSCLC comprising selecting one or more NSCLC treatments described herein and/or administering to the individual one or more NSCLC treatments described herein. In some embodiments, the kit comprises instructions for use according to any of the methods provided herein.

### VIII.B. Peptide Structure and Product Ion Compositions and Kits—Ovarian Cancer

Aspects of the disclosure include compositions comprising one or more of the peptide structures listed in Table 41, in Table 42, in Table 43A, in Table 43B, in Table 43C, or in Table 43D. In some embodiments, a composition comprises a plurality of the peptide structures listed in Table 41, a plurality of the peptide structures listed in Table 42, or a plurality of the peptide structures listed in Table 43A. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 of the peptide structures listed in Tables 41, 42, 43A, 43B, 43C, and 43D. In one or more embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36 of the peptide structures listed in Table 43B. In one or more embodiments, a composition comprises 1, 2, 3, 4, 5, 6, 7, 8, 9, 10, 11, 12, 13, 14, 15, 16, 17, 18, 19, 20, 21, 22, 23, 24, or all 25 of the glycopeptide structures listed in Table 43C. In one or more embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all 50 of the glycopeptide structures listed in Table 43D.

In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 307-315, 428-443, 450-462, 465-474, 475-499, 500-549 listed in Tables 41, 42, 43A, 43B, 43C, and 43D.

Aspects of the disclosure include compositions comprising one or more precursor ions having a defined charge and/or defined mass-to-charge (m/z) ratio, as listed in Tables 44A, 44B, and 44C. Aspects of the disclosure include compositions comprising one or more product ions having a defined mass-to-charge (m/z) ratio, which product ions are produced by converting a peptide structure described herein (e.g., a peptide structure listed in Tables 41, 42, 43A, 43B, 43C, or 43D) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Tables 41, 42, 43A, 43B, 43C, or 43D). In some embodiments, a composition comprises a set of the product ions listed in Table 44A, 44B, or 44C having an m/z ratio selected from the list provided for each peptide structure in Table 44A, 44B, and 44C.

In some embodiments, a composition comprises at least one of peptide structures PS-165 to PS-174 identified in Table 41. In some embodiments, a composition comprises at least one of peptide structures PS-175 to PS-198 and PS-169 identified in Table 42. In some embodiments, a composition comprises at least one of peptide structures PS-165, PS-169, PS-175, PS-179, PS-184, PS-189, PS-192, PS-193, PS-194, PS-195, PS-196, and PS-199 to PS-225 identified in Table 43A. In some embodiments, a composition comprises at least one of peptide structures PS-168, PS-172, PS-182, PS-200, PS-201, PS-205, PS-220, PS-226 to PS-254 identified in Table 43B. In some embodiments, a composition comprises at least one of peptide structures of SEQ ID NOS 475-499 identified in Table 43C. In some embodiments, a composition comprises at least one of peptide structures of PS-ID 280 to 329 identified in Table 43D.

In one or more embodiments, a composition comprises at least 1, at least 2, at least 3, at least 4, at least 5, at least 6, at least 7, at least 8, at least 9, or all 10 of the peptide structures PS-165 to PS-174 identified in Table 41. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures PS-175 to PS-198 and PS-169 identified in Table 42. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures PS-165, PS-169, PS-175, PS-179, PS-184, PS-189, PS-192, PS-193, PS-194, PS-195, PS-196, and PS-199 to PS-225 identified in Table 43A. In some embodiments, the at least 3 peptide structures additionally include at least 1, at least 2, at least 3, at least 4, at least 5, at least 6, or all 7 of the remaining peptide structures PS-165, PS-169, PS-175, PS-179, PS-184, PS-189, PS-192, PS-193, PS-194, PS-195, PS-196, and PS-199 to PS-225 identified in Table 43A. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, or all 36 of the peptide structures PS-168, PS-172, PS-182, PS-200, PS-201, PS-206, PS-220, PS-226 to PS-254 identified in Table 43B. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures of SEQ ID NOS 495-499 identified in Table 43C. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or all 50 of the peptide structures of SEQ ID NOS 500-549 identified in Table 43D.

In some embodiments, a composition comprises a peptide structure or a product ion. The peptide structure or product ion can include an amino acid sequence having at least 90% sequence identity to any one of SEQ ID NOS: 307-315, as identified in Table 45, corresponding to peptide structures PS-165 to PS-174 in Table 41. The peptide structure or product ion can include an amino acid sequence having at least 90% sequence identity to any one of SEQ ID NOS: 309, 311, 428-443, as identified in Table 45, corresponding to various ones of peptide structures PS-169 and PS-175 to PS-198 in Table 42. The peptide structure or product ion can include an amino acid sequence having at least 90% sequence identity to any one of SEQ ID NOS: 11, 14, 15, 31, 32, 33, 34, 37, 38, 40, 42, 44, 45, 46, 53-65307, 310, 311, 428-431, 434, 435, 437, 439, 441, 442, 443, 450-462, as identified in Table 45, corresponding to various ones of peptide structures PS-165, PS-169, PS-175, PS-179, PS-184, PS-189, PS-192, PS-193, PS-194, PS-195, PS-196, and PS-199 to PS-225 in Table 43A. The peptide structure or product ion can include an amino acid sequence having at least 90% sequence identity to any one of SEQ ID NOS: 310, 314, 429, 430, 434, 436, 439, 442, 451, 453, 457, 465, 466, 467, 468, 469, 470, 471, 472, 473, and 474, as identified in Table 45, corresponding to various ones of peptide structures PS-168, PS-172, PS-182, PS-200, PS-201, PS-205, PS-220, PS-226 to PS-254 in Table 43B. The peptide structure or product ion can include an amino acid sequence having at least 90% sequence identity to any one of SEQ ID NOS: 475-499, corresponding to various ones of peptide structures in Table 43C or product ions in Table 44B. The peptide structure or product ion can include an amino acid sequence having at least 90% sequence identity to any one of SEQ ID NOS: 500-549, as identified in Table 45, corresponding to various ones of peptide structures PS-280 to PS-329 in Table 43D.

In some embodiments, the product ion is selected as one from a group consisting of product ions identified in Tables 44A, 44B, and 44C including product ions falling within an identified m/z range of the m/z ratio identified in Tables 44A, 44B, and 44C and characterized as having a precursor ion having an m/z ratio within an identified m/z range of the m/z ratio identified in Tables 44A, 44B, and 44C. A first range for the product ion m/z ratio may be +0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (1.0) of the product ion m/z ratio identified in Tables 44A, 44B, and 44C, and characterized as having a precursor ion having an m/z ratio that falls within at least one of first range (±0.5), a second range (±1.0), or a third range (±1.0 of the precursor ion m/z ratio identified in Tables 44A, 44B, and 44C.

TABLE 44A
Mass Spectrometry-Related Characteristics for the Peptide
Structures associated with Ovarian Cancer (e.g., EOC)

| | | | | | | 2^{nd} | |
|---|---|---|---|---|---|---|---|
| | | Collision | 1^{st} | 1^{st} | 1^{st} | Collision | 2^{nd} |
| PS-ID | RT | Energy | Precursor | Precursor | Product | Energy | Product |
| NO. | (min) | (V) | m/z | Charge | m/z | (V) | m/z |
| 165 | 10.6 | 30 | 1115.1 | 3 | 366.1 | 34 | 1341.6 |
| 166 | 35.8 | 35 | 1241.8 | 4 | 204.1 | 20 | 1152.6 |
| 167 | 6.6 | 25 | 1009.4 | 3 | 366.1 | N/A | N/A |
| 168 | 17.1 | 30 | 1226.2 | 4 | 366.1 | 30 | 1048.5 |
| 169 | 7.9 | 21 | 879 | 3 | 204.1 | 27 | 1392.6 |
| 170 | 40.5 | 35 | 1184.5 | 4 | 204.1 | N/A | N/A |
| 171 | 33.6 | 30 | 1440.3 | 4 | 366.1 | N/A | N/A |
| 172 | 13.3 | 35 | 1378.9 | 5 | 366.1 | N/A | N/A |
| 173 | 10.1 | 35 | 1237 | 2 | 204.1 | 20 | 1376.6 |
| 174 | 10.1 | 35 | 1237 | 2 | 204.1 | 20 | 1376.6 |
| 175 | 16.3 | 20 | 891.1 | 3 | 829.4 | 20 | 366.1 |
| 176 | 22.6 | 31 | 1250.3 | 4 | 366.1 | N/A | N/A |
| 177 | 30.2 | 28 | 1173.2 | 4 | 366.1 | 978.5 | 25 |
| 178 | 44 | 15 | 874.4 | 5 | 366.1 | 1183.6 | 20 |
| 179 | 31.3 | 30 | 1191.2 | 4 | 366.1 | 978.5 | 20 |
| 180 | 27.4 | 35 | 1084.2 | 4 | 204.1 | N/A | N/A |
| 181 | 12.6 | 28 | 1106.8 | 3 | 366.1 | N/A | N/A |
| 182 | 37.8 | 30 | 1082.6 | 5 | 274.1 | N/A | N/A |
| 183 | 16.7 | 20 | 1025.7 | 4 | 274.1 | 1048.5 | 25 |
| 184 | 43.3 | 34 | 1341 | 5 | 366.1 | 1299 | 34 |
| 185 | 22.6 | 30 | 1213.8 | 4 | 366.1 | N/A | N/A |
| 186 | 37.3 | 30 | 1336.3 | 4 | 366.1 | N/A | N/A |
| 187 | 13.1 | 13 | 935.8 | 3 | 204.1 | 1360.6 | 30 |
| 188 | 14.8 | 25 | 1021.4 | 4 | 366.1 | N/A | N/A |
| 189 | 5.7 | 29 | 1165.6 | 4 | 366.1 | 979.5 | 29 |
| 190 | 7.9 | 30 | 1224.5 | 2 | 366.1 | N/A | N/A |
| 191 | 18.5 | 33 | 1055.8 | 3 | 366.1 | 1453.6 | 35 |
| 192 | 23.5 | 20 | 1079.7 | 4 | 366.1 | N/A | N/A |
| 193 | 31 | 30 | 1144.9 | 4 | 366.1 | 1359.6 | 35 |
| 194 | 16.5 | 34 | 1117.2 | 4 | 366.1 | N/A | N/A |
| 195 | 43.5 | 22 | 1057 | 4 | 366.1 | 1184.1 | 28 |
| 196 | 41.5 | 22 | 1115.4 | 4 | 366.1 | 366.1 | 25 |
| 197 | 32.3 | 30 | 1217.7 | 4 | 366.1 | 1359.6 | 35 |
| 198 | 13.6 | 35 | 1087.1 | 3 | 204.1 | N/A | N/A |
| 199 | 24.3 | 23 | 942.4 | N/A | 366.1 | N/A | N/A |
| 200 | 31.1 | 34 | 1343.8 | N/A | 366.1 | N/A | N/A |
| 201 | 23.9 | 25 | 1116.9 | N/A | 366.1 | N/A | N/A |
| 202 | 31.3 | 15 | 590.3 | N/A | 725.4 | N/A | N/A |
| 203 | 34.2 | 25 | 1149.3 | N/A | 366.1 | N/A | N/A |
| 204 | 28 | 27 | 1085.4 | N/A | 366.1 | N/A | N/A |
| 205 | 33.8 | 27 | 1105.6 | N/A | 366.1 | N/A | N/A |
| 206 | 31.2 | 30 | 1314.9 | N/A | 366.1 | N/A | N/A |
| 207 | 34.4 | 25 | 819.1 | N/A | 855.5 | N/A | N/A |
| 208 | 31 | 25 | 1035.6 | N/A | 366.1 | N/A | N/A |
| 209 | 5.6 | 25 | 1256.8 | N/A | 366.1 | N/A | N/A |
| 210 | 26.4 | 20 | 1252.5 | N/A | 366.1 | N/A | N/A |
| 211 | 31 | 33 | 1335.3 | N/A | 366.1 | N/A | N/A |
| 212 | 8.1 | 20 | 1054.7 | N/A | 366.1 | N/A | N/A |
| 213 | 40.3 | 29 | 944.5 | N/A | 1269.6 | N/A | N/A |
| 214 | 13.1 | 25 | 1043.8 | N/A | 366.1 | N/A | N/A |
| 215 | 5.8 | 34 | 1335 | N/A | 366.1 | N/A | N/A |
| 216 | 13.2 | 25 | 927.7 | N/A | 366.1 | N/A | N/A |
| 217 | 33 | 25 | 1018.1 | N/A | 366.1 | N/A | N/A |
| 218 | 27.4 | 25 | 1012.7 | N/A | 366.1 | N/A | N/A |
| 219 | 13.2 | 15 | 989.9 | N/A | 204.1 | N/A | N/A |
| 220 | 38.6 | 35 | 1214.1 | N/A | 274.1 | N/A | N/A |
| 221 | 40 | 20 | 693.9 | N/A | 675.4 | N/A | N/A |
| 222 | 30.4 | 26 | 1070.4 | N/A | 366.1 | N/A | N/A |
| 223 | 23 | 20 | 988.8 | N/A | 274.1 | N/A | N/A |
| 224 | 15.7 | 12 | 453.2 | N/A | 532.2 | N/A | N/A |
| 225 | 37.5 | 25 | 1116.9 | N/A | 366.1 | N/A | N/A |
| 226 | 37.9 | 30 | 1053.4 | 5 | 274.1 | N/A | N/A |
| 227 | 38.3 | 30 | 1184.9 | 5 | 274.1 | N/A | N/A |
| 228 | 30.8 | 35 | 1441.6 | 3 | 366.1 | 30 | 978.5 |
| 229 | 5.8 | 30 | 1213.3 | 3 | 366.1 | 30 | 979.5 |
| 230 | 5.7 | 32 | 1262 | 3 | 366.1 | 32 | 979.5 |
| 231 | 5.4 | 31 | 1238 | 3 | 366.1 | 31 | 979.5 |
| 232 | 5.9 | 27 | 1110.8 | 4 | 366.1 | 27 | 979.5 |
| 233 | 23.4 | 25 | 1195.3 | 4 | 366.1 | N/A | N/A |
| 234 | 23.3 | 31 | 1231.8 | 4 | 274.1 | N/A | N/A |
| 235 | 23.1 | 33 | 1323.1 | 4 | 366.1 | N/A | N/A |
| 236 | 5.4 | 35 | 1220.1 | 3 | 274.1 | 35 | 999.4 |
| 237 | 5.3 | 33 | 1268.8 | 3 | 274.1 | 33 | 999.4 |
| 238 | 37.7 | 30 | 1196.5 | 4 | 366.1 | 20 | 1062.5 |
| 239 | 37.5 | 25 | 1287.8 | 4 | 366.1 | 25 | 1062.5 |
| 240 | 4.6 | 33 | 1257.5 | 3 | 366.1 | 33 | 965.5 |
| 241 | 19.6 | 25 | 1028.9 | 4 | 274.1 | 25 | 1453.6 |
| 242 | 27.5 | 35 | 1284.8 | 4 | 204.1 | N/A | N/A |
| 243 | 20.8 | 30 | 1258.5 | 4 | 274.1 | 30 | 1113 |
| 244 | 27.7 | 29 | 1159.5 | 4 | 366.1 | 25 | 274.1 |
| 245 | 21.6 | 30 | 1067.7 | 4 | 366.1 | N/A | N/A |
| 246 | 21.5 | 25 | 1104.2 | 4 | 274.1 | N/A | N/A |
| 247 | 34.5 | 28 | 1138.4 | 5 | 366.1 | 35 | 1441.7 |
| 248 | 13.2 | 31 | 1247.7 | 5 | 366.1 | N/A | N/A |
| 249 | 13.4 | 34 | 1335.1 | 5 | 366.1 | N/A | N/A |
| 250 | 30.9 | 30 | 1201.5 | 4 | 366.1 | N/A | N/A |
| 251 | 30.7 | 32 | 1292.8 | 4 | 366.1 | 32 | 999.5 |
| 252 | 16.9 | 25 | 1106.4 | 4 | 274.1 | N/A | N/A |
| 253 | 23.1 | 20 | 1074.4 | 4 | 274.1 | N/A | N/A |
| 254 | 37.7 | 25 | 1127.9 | 5 | 366.1 | N/A | N/A |

TABLE 44B
Mass Spectrometry-Related Characteristics for the Peptide Structures associated
with Ovarian Cancer (e.g., EOC) - in accordance with Table 43C

| SEQ | | Collision | 1st | 1st | 2nd | 2nd | 1st | 2nd |
|---|---|---|---|---|---|---|---|---|
| ID | RT | Energy | Precursor | Precursor | Precursor | Precursor | Product | Product |
| NO | (min) | (V) | m/z | Charge | m/z | Charge | m/z | m/z |
| 475 | 30.6 | 30 | 1118.2 | 4 | 1118.2 | 4 | 366.1 | 978.5 |
| 476 | 5.8 | 30 | 1213.3 | 3 | 1213.3 | 3 | 366.1 | 979.5 |
| 477 | 5.7 | 32 | 1262 | 3 | 1262 | 3 | 366.1 | 979.5 |
| 478 | 5.9 | 27 | 1110.8 | 4 | 1110.8 | 4 | 366.1 | 979.5 |
| 479 | 23.3 | 31 | 1231.8 | 4 | n/a | n/a | 274.1 | n/a |
| 480 | 23.8 | 27 | 1087.7 | 5 | n/a | n/a | 366.1 | n/a |
| 481 | 23.1 | 33 | 1323.1 | 4 | n/a | n/a | 366.1 | n/a |
| 482 | 23.8 | 25 | 1116.9 | 5 | 1116.9 | 5 | 366.1 | 1060 |
| 483 | 5.4 | 35 | 1220.1 | 3 | 1220.1 | 3 | 274.1 | 999.4 |
| 484 | 37.7 | 30 | 1196.5 | 4 | 1196.5 | 4 | 366.1 | 1062.5 |
| 485 | 37.8 | 30 | 1360.5 | 4 | 1360.5 | 4 | 366.1 | 1062.5 |
| 486 | 41.3 | 32 | 1283.7 | 5 | 1283.7 | 5 | 366.1 | 1550.3 |
| 487 | 41.2 | 27 | 1313.1 | 5 | 1313.1 | 5 | 366.1 | 1550.3 |
| 488 | 13.1 | 25 | 1083.4 | 4 | n/a | n/a | 274.1 | n/a |
| 489 | 20.8 | 30 | 1258.5 | 4 | 1258.5 | 4 | 274.1 | 1113 |
| 490 | 21.6 | 30 | 1067.7 | 4 | n/a | n/a | 366.1 | n/a |
| 491 | 34.5 | 28 | 1138.4 | 5 | 1138.4 | 5 | 366.1 | 1441.7 |
| 492 | 13.4 | 34 | 1335.1 | 5 | n/a | n/a | 366.1 | n/a |
| 493 | 13.3 | 35 | 1378.9 | 5 | n/a | n/a | 366.1 | n/a |
| 494 | 31.1 | 29 | 1165 | 4 | n/a | n/a | 366.1 | n/a |
| 495 | 30.9 | 30 | 1201.5 | 4 | n/a | n/a | 366.1 | n/a |
| 496 | 30.7 | 32 | 1292.8 | 4 | 1292.8 | 4 | 366.1 | 999.5 |
| 497 | 16.9 | 25 | 1106.4 | 4 | n/a | n/a | 274.1 | n/a |
| 498 | 23.1 | 20 | 1074.4 | 4 | n/a | n/a | 274.1 | n/a |
| 499 | 33.2 | 27 | 1105.6 | 5 | 1105.6 | 5 | 366.1 | 1359.6 |

TABLE 44C
Mass Spectrometry-Related Characteristics for the Peptide Structures associated
with Ovarian Cancer (e.g., EOC) - in accordance with Table 43D

| | | Collision | 1st | 1st | 2nd | 2nd | 1st | 2nd |
|---|---|---|---|---|---|---|---|---|
| SEQ | RT | Energy | Precursor | Precursor | Precursor | Precursor | Product | Product |
| ID NO | (min) | (V) | m/z | Charge | m/z | Charge | m/z | m/z |
| 500 | 42.7 | 30 | 1282.9 | 5 | N/A | N/A | 366.1 | 1299 |
| 501 | 43.4 | 27 | 1111.7 | 4 | N/A | N/A | 366.1 | 1183.6 |
| 502 | 43.3 | 23 | 1148.3 | 4 | N/A | N/A | 366.1 | 1183.6 |
| 503 | 37.9 | 30 | 1053.4 | 5 | N/A | N/A | 274.1 | N/A |
| 504 | 38.3 | 30 | 1184.9 | 5 | N/A | N/A | 274.1 | N/A |
| 505 | 38.3 | 35 | 1214.1 | 5 | N/A | N/A | 274.1 | N/A |
| 506 | 30.8 | 35 | 1441.6 | 3 | N/A | N/A | 366.1 | 978.5 |
| 507 | 31.4 | 25 | 1154.7 | 4 | N/A | N/A | 274.1 | 978.5 |
| 508 | 5.4 | 35 | 1220.1 | 3 | N/A | N/A | 274.1 | 999.4 |
| 509 | 41 | 28 | 1152.7 | 5 | N/A | N/A | 366.1 | 1550.3 |
| 510 | 41 | 29 | 1181.9 | 5 | N/A | N/A | 366.1 | 1550.3 |
| 511 | 40.9 | 30 | 1568.4 | 4 | 1254.9 | 5 | 366.1 | 366.1 |
| 512 | 41.2 | 27 | 1313.1 | 5 | N/A | N/A | 366.1 | 1550.3 |
| 513 | 37.7 | 30 | 1196.5 | 4 | N/A | N/A | 366.1 | 1062.5 |
| 514 | 37.5 | 25 | 1287.8 | 4 | N/A | N/A | 366.1 | 1062.5 |
| 515 | 37.8 | 30 | 1360.5 | 4 | N/A | N/A | 366.1 | 1062.5 |
| 516 | 5.8 | 30 | 1213.3 | 3 | N/A | N/A | 366.1 | 979.5 |
| 517 | 5.7 | 32 | 1262 | 3 | N/A | N/A | 366.1 | 979.5 |
| 518 | 5.4 | 31 | 1238 | 3 | N/A | N/A | 366.1 | 979.5 |
| 519 | 5.9 | 26 | 1074.3 | 4 | N/A | N/A | 366.1 | 979.5 |
| 520 | 5.9 | 27 | 1110.8 | 4 | N/A | N/A | 366.1 | 979.5 |
| 521 | 23.4 | 25 | 1195.3 | 4 | N/A | N/A | 366.1 | N/A |
| 522 | 22.6 | 30 | 1213.8 | 4 | N/A | N/A | 366.1 | N/A |
| 523 | 23.2 | 25 | 1286.6 | 4 | N/A | N/A | 366.1 | N/A |
| 524 | 23.8 | 27 | 1087.7 | 5 | N/A | N/A | 366.1 | N/A |
| 525 | 22.6 | 31 | 1250.3 | 4 | N/A | N/A | 366.1 | N/A |
| 526 | 23.1 | 33 | 1323.1 | 4 | N/A | N/A | 366.1 | N/A |
| 527 | 23.8 | 25 | 1116.9 | 5 | N/A | N/A | 366.1 | 1060 |
| 528 | 4.8 | 30 | 1208.6 | 3 | N/A | N/A | 366.1 | 965.5 |
| 529 | 4.6 | 33 | 1257.5 | 3 | N/A | N/A | 366.1 | 965.5 |
| 530 | 4.8 | 29 | 1070.7 | 4 | N/A | N/A | 366.1 | 965.5 |
| 531 | 13.1 | 25 | 1083.4 | 4 | N/A | N/A | 274.1 | N/A |
| 532 | 19.1 | 30 | 1201.5 | 3 | N/A | N/A | 274.1 | 1453.6 |
| 533 | 27.5 | 35 | 1284.8 | 4 | N/A | N/A | 204.1 | N/A |
| 534 | 20.8 | 25 | 1222 | 4 | N/A | N/A | 274.1 | 1113 |
| 535 | 27.7 | 29 | 1159.5 | 4 | N/A | N/A | 366.1 | 274.1 |
| 536 | 27.6 | 30 | 1196 | 4 | N/A | N/A | 366.1 | 274.1 |
| 537 | 30.4 | 34 | 1343.8 | 4 | N/A | N/A | 366.1 | N/A |
| 538 | 21.6 | 30 | 1067.7 | 4 | N/A | N/A | 366.1 | N/A |
| 539 | 21.5 | 25 | 1104.2 | 4 | N/A | N/A | 274.1 | N/A |
| 540 | 34.5 | 28 | 1138.4 | 5 | N/A | N/A | 366.1 | 1441.7 |
| 541 | 13.2 | 31 | 1247.7 | 5 | N/A | N/A | 366.1 | N/A |
| 542 | 13.1 | 32 | 1276.9 | 5 | N/A | N/A | 366.1 | N/A |
| 543 | 13.4 | 34 | 1335.1 | 5 | N/A | N/A | 366.1 | N/A |
| 544 | 13.3 | 35 | 1378.9 | 5 | N/A | N/A | 366.1 | N/A |
| 545 | 28.9 | 25 | 1055.7 | 4 | N/A | N/A | 366.1 | 999.5 |
| 546 | 30 | 28 | 1128.8 | 4 | N/A | N/A | 366.1 | N/A |
| 547 | 30.9 | 30 | 1201.5 | 4 | N/A | N/A | 366.1 | N/A |
| 548 | 30.7 | 32 | 1292.8 | 4 | N/A | N/A | 366.1 | 999.5 |
| 549 | 23.1 | 20 | 1074.4 | 4 | N/A | N/A | 274.1 | N/A |

Tables 44A, 44B, and 44C show various parameters associated with the identification of the peptide and glycopeptides using LC and MRM-MS. The retention time (RT) represents the amount of time in minutes for the peptide elute from the chromatography column. The collision energy represents the energy applied to the peptide for creating fragments (i.e., product ions) such as, for example, in the 2^{nd} quadrupole of the triple quadrupole MS. The first precursor m/z represents a ratio value associated with an ionized form having a first precursor charge for the peptide or glycopeptide. Similarly, the second precursor m/z represents a ratio value associated with an ionized form having a second precursor charge for the peptide or glycopeptide. The first precursor ion is associated with a first product ion having a m/z ratio that was formed from a collision and the second precursor ion is associated with a second product ion having a m/z ratio that was formed from a collision. Under certain circumstances, the first precursor and the second precursor may be the same, but the associated first and second product m/z ratios are different.

Table 45 defines the peptide sequences for SEQ ID NOS: 307-316, 428-443, 450-462, 465-474, and 500-549 from at least one of Tables 41, 42, 43A, 43B, 43C, and 43D. Table 45 further identifies a corresponding protein SEQ TD NO. for each peptide sequence.

TABLE 45
Peptide SEQ ID NOS

| Pept | | Corresponding |
|---|---|---|
| SEQ ID | | Protein |
| NO: | Peptide Sequence | SEQ ID NO: |
| 307 | FGCEIENNR | 297 |
| 308 | VLSNNSDANLELINTWVAK | 298 |
| 309 | LISNCSK | 299 |
| 310 | EHEGAIYPDNTTDFQR | 300 |
| 311 | EEQYNSTYR | 301 |
| 312 | CSDGWSFDATTLDDNGTMLFFK | 302 |
| 313 | QVFPGLNYCTSGAYSNASSTDSASYYPLTGDTR | 303 |
| 314 | NLFLNHSENATAK | 304 |
| 315 | EEQYNSTFR | 305, 306 |
| 316 | EEQYNSTFR | 306 |
| 428 | QSVPAHFVALNGSK | 417 |
| 429 | QDQCIYNTTYLNVQR | 418 |
| 430 | YTGNASALFILPDQDK | 419 |
| 431 | VSNQTLSLFFTVLQDVPVR | 420 |
| 432 | ENLTAPGSDSAVFFEQGTTR | 300 |
| 433 | FVEGSHNSTVSLTTK | 303 |
| 434 | FNLTETSEAEIHQSFQHLLR | 419 |
| 435 | ADTHDEILEGLNFNLTEIPEAQIHEGFQELLR | 421 |
| 436 | NISDGFDGIPDNVDAALALPAHSYSGR | 422 |
| 437 | EEQFNSTFR | 423 |
| 438 | IPCSQPPQIEHGTINSSR | 424 |
| 439 | ENGTISR | 18 |
| 440 | LGNWSAMPSCK | 425 |
| 441 | ADGTVNQIEGEATPVNLTEPAK | 426 |
| 442 | QQQHLFGSNVTDCSGNFCLFR | 427 |
| 443 | GCVLLSYLNETVTVSASLESVR | 420 |
| 450 | NGSLFAFR | 422 |
| 451 | AALAAFNAQNNGSNFQLEEISR | 444 |
| 452 | DLLLPQPDLR | 445 |
| 453 | MVSHHNLTTGATLINEQWLLTTAK | 304 |
| 454 | CGLVPVLAENYNK | 427 |
| 455 | ALPQPQNVTSLLGCTH | 302 |
| 456 | TSESGELHGLTTEEEFVEGIYK | 446 |
| 457 | VVLHPNYSQVDIGLIK | 304 |
| 458 | SDVGFLPPFPTLDPEEK | 447 |
| 459 | VSFLSALEEYTK | 448 |
| 460 | TVVQPSVGAAAGPVVPPCPGR | 444 |
| 461 | THLAPYSDELR | 448 |
| 462 | FSLLGHASISCTVENETIGVWRPSPPTCEK | 449 |
| 465 | NEEYNK | 418, 463 |
| 466 | SVQEIQATFFYFTPNK | 418, 463 |
| 467 | ENGTVSR | 463 |
| 468 | LGNWSAMPSCK | 425 |
| 469 | ENLTAPGSDSAVFFEQGTTR | 300 |
| 470 | ELHHLQEQNVSNAFLDK | 300 |
| 471 | VCQDCPLLAPLNDTR | 444 |
| 472 | SWPAVGNCSSALR | 302 |
| 473 | SWPAVGNCSSALR | 302 |
| 474 | ITYSIVQTNCSK | 464 |
| 465 | LNAENNATFYFK | 464 |
| 500 | ADTHDEILEGLNFNLTEIPEAQIHEGFQELLR | 421 |
| 501 | VSNQTLSLFFTVLQDVPVR | 420 |
| 502 | VSNQTLSLFFTVLQDVPVR | 420 |
| 503 | FNLTETSEAEIHQSFQHLLR | 419 |
| 504 | FNLTETSEAEIHQSFQHLLR | 419 |
| 505 | FNLTETSEAEIHQSFQHLLR | 419 |
| 506 | YTGNASALFILPDQDK | 419 |
| 507 | YTGNASALFILPDQDK | 419 |
| 508 | NEEYNK | 418 or 463 |
| 509 | SVQEIQATFFYFTPNKTEDTIFLR | 418 or 463 |
| 510 | SVQEIQATFFYFTPNKTEDTIFLR | 418 or 463 |
| 511 | SVQEIQATFFYFTPNKTEDTIFLR | 418 or 463 |
| 512 | SVQEIQATFFYFTPNKTEDTIFLR | 418 or 463 |
| 513 | SVQEIQATFFYFTPNK | 418 or 463 |
| 514 | SVQEIQATFFYFTPNK | 418 or 463 |
| 515 | SVQEIQATFFYFTPNK | 418 or 463 |
| 516 | ENGTISR | 418 |
| 517 | ENGTISR | 418 |
| 518 | ENGTISR | 418 |
| 519 | ENGTISR | 418 |
| 520 | ENGTISR | 418 |
| 521 | QDQCIYNTTYLNVQR | 418 |
| 522 | QDQCIYNTTYLNVQR | 418 |
| 523 | QDQCIYNTTYLNVQR | 418 |
| 524 | QDQCIYNTTYLNVQR | 418 |
| 525 | QDQCIYNTTYLNVQR | 418 |
| 526 | QDQCIYNTTYLNVQR | 418 |
| 527 | QDQCIYNTTYLNVQR | 418 |
| 528 | ENGTVSR | 463 |
| 529 | ENGTVSR | 463 |
| 530 | ENGTVSR | 463 |
| 531 | VYKPSAGNNSLYR | 425 |
| 532 | LGNWSAMPSCK | 425 |
| 533 | ENLTAPGSDSAVFFEQGTTR | 300 |
| 534 | ELHHLQEQNVSNAFLDK | 300 |
| 535 | VCQDCPLLAPLNDTR | 444 |
| 536 | VCQDCPLLAPLNDTR | 444 |
| 537 | AALAAFNAQNNGSNFQLEEISR | 444 |
| 538 | SWPAVGNCSSALR | 302 |
| 539 | SWPAVGNCSSALR | 302 |
| 540 | MVSHHNLTTGATLINEQWLLTTAK | 304 |
| 541 | NLFLNHSENATAK | 304 |
| 542 | NLFLNHSENATAK | 304 |
| 543 | NLFLNHSENATAK | 304 |
| 544 | NLFLNHSENATAK | 304 |
| 545 | VVLHPNYSQVDIGLIK | 304 |
| 546 | VVLHPNYSQVDIGLIK | 304 |
| 547 | VVLHPNYSQVDIGLIK | 304 |
| 548 | VVLHPNYSQVDIGLIK | 304 |
| 549 | LNAENNATFYFK | 464 |

Table 46 identifies the proteins of SEQ ID NOS: 297-306, 417-427, 444-449, and 463-464 from at least of one of Tables 41, 42, 43A, 43B, 43C, and 43D. Table 46 identifies a corresponding protein abbreviation and protein name for each of protein SEQ TD NOS: 1-10, 20-30, 47-52, and 66-67. Further, Table 46 identifies a corresponding Uniprot TD and protein sequence for each of protein SEQ ID NOS: 297-306, 417-427, 444-449, and 463-464.

TABLE 46
Protein SEQ ID NOS

| SEQ | | | | |
|---|---|---|---|---|
| ID | Protein | | Uniprot | |
| NO. | Abbreviation | Protein Name | ID | Protein Sequence |
| 297 | ZA2G | Zinc-alpha-2- | P25311 | MVRMVPVLLSLLLLLGPAVPQENQDGR |
| | | glycoprotein | | YSLTYIYTGLSKHVEDVPAFQALGSLN |
| | | | | DLQFFRYNSKDRKSQPMGLWRQVEGM |
| | | | | EDWKQDSQLQKAREDIFMETLKDIVEY |
| | | | | YNDSNGSHVLQGRFGCEIENNRSSGAF |
| | | | | WKYYYDGKDYIEFNKEIPAWVPFDPAA |
| | | | | QITKQKWEAEPVYVQRAKAYLEEECPA |
| | | | | TLRKYLKYSKNILDRQDPPSVVVTSHQ |
| | | | | APGEKKKLKCLAYDFYPGKIDVHWTR |
| | | | | AGEVQEPELRGDVLHNGNGTYQSWVV |
| | | | | VAVPPQDTAPYSCHVQHSSLAQPLVVP |
| | | | | WEAS |
| 298 | IC1 | Plasma | P05155 | MASRLTLLTLLLLLLAGDRASSNPNATS |
| | | protease C1 | | SSSQDPESLQDRGEGKVATTVISKMLFV |
| | | inhibitor | | EPILEVSSLPTTNSTTNSATKITANTTDE |
| | | | | PTTQPTTEPTTQPTIQPTQPTTQLPTDSP |
| | | | | TQPTTGSFCPGPVTLCSDLESHSTEAVL |
| | | | | GDALVDFSLKLYHAFSAMKKVETNMA |
| | | | | FSPFSIASLLTQVLLGAGENTKTNLESIL |
| | | | | SYPKDFTCVHQALKGFTTKGVTSVSQIF |
| | | | | HSPDLAIRDTFVNASRTLYSSSPRVLSN |
| | | | | NSDANLELINTWVAKNTNNKISRLLDS |
| | | | | LPSDTRLVLLNAIYLSAKWKTTFDPKKT |
| | | | | RMEPFHFKNSVIKVPMMNSKKYPVAHF |
| | | | | IDQTLKAKVGQLQLSHNLSLVILVPQNL |
| | | | | KHRLEDMEQALSPSVFKAIMEKLEMSK |
| | | | | FQPTLLTLPRIKVTTSQDMLSIMEKLEFF |
| | | | | DFSYDLNLCGLTEDPDLQVSAMQHQTV |
| | | | | LELTETGVEAAAASAISVARTLLVFEVQ |
| | | | | QPFLFVLWDQQHKFPVFMGRVYDPRA |
| 299 | CFAI | Complement | P05156 | MKLLHVFLLFLCFHLRFCKVTYTSQED |
| | | Factor I | | LVEKKCLAKKYTHLSCDKVFCQPWQR |
| | | | | CIEGTCVCKLPYQCPKNGTAVCATNRR |
| | | | | SFPTYCQQKSLECLHPGTKFLNNGTCTA |
| | | | | EGKFSVSLKHGNTDSEGIVEVKLVDQD |
| | | | | KTMFICKSSWSMREANVACLDLGFQQ |
| | | | | GADTQRRFKLSDLSINSTECLHVHCRGL |
| | | | | ETSLAECTFTKRRTMGYQDFADVVCYT |
| | | | | QKADSPMDDFFQCVNGKYISQMKACD |
| | | | | GINDCGDQSDELCCKACQGKGFHCKSG |
| | | | | VCIPSQYQCNGEVDCITGEDEVGCAGF |
| | | | | ASVTQEETEILTADMDAERRRIKSLLPK |
| | | | | LSCGVKNRMHIRRKRIVGGKRAQLGDL |
| | | | | PWQVAIKDASGITCGGIYIGGCWILTAA |
| | | | | HCLRASKTHRYQIWTTVVDWIHPDLKR |
| | | | | IVIEYVDRIIFHENYNAGTYQNDIALIEM |
| | | | | KKDGNKKDCELPRSIPACVPWSPYLFQ |
| | | | | PNDTCIVSGWGREKDNERVFSLQWGEV |
| | | | | KLISNCSKFYGNRFYEKEMECAGTYDG |
| | | | | SIDACKGDSGGPLVCMDANNVTYVWG |
| | | | | VVSWGENCGKPEFPGVYTKVANYFDW |
| | | | | ISYHVGRPFISQYNV |
| 300 | CERU | Ceruloplasmin | P00450 | MKILILGIFLFLCSTPAWAKEKHYYIGII |
| | | | | ETTWDYASDHGEKKLISVDTEHSNIYL |
| | | | | QNGPDRIGRLYKKALYLQYTDETFRTTI |
| | | | | EKPVWLGFLGPIIKAETGDKVYVHLKN |
| | | | | LASRPYTFHSHGITYYKEHEGAIYPDNT |
| | | | | TDFQRADDKVYPGEQYTYMLLATEEQ |
| | | | | SPGEGDGNCVTRIYHSHIDAPKDIASGLI |
| | | | | GPLIICKKDSLDKEKEKHIDREFVVMFS |
| | | | | VVDENFSWYLEDNIKTYCSEPEKVDKD |
| | | | | NEDFQESNRMYSVNGYTFGSLPGLSMC |
| | | | | AEDRVKWYLFGMGNEVDVHAAFFHG |
| | | | | QALTNKNYRIDTINLFPATLFDAYMVA |
| | | | | QNPGEWMLSCQNLNHLKAGLQAFFQV |
| | | | | QECNKSSSKDNIRGKHVRHYYIAAEEII |
| | | | | WNYAPSGIDIFTKENLTAPGSDSAVFFE |
| | | | | QGTTRIGGSYKKLVYREYTDASFTNRK |
| | | | | ERGPEEEHLGILGPVIWAEVGDTIRVTF |
| | | | | HNKGAYPLSIEPIGVRFNKNNEGTYYSP |
| | | | | NYNPQSRSVPPSASHVAPTETFTYEWT |
| | | | | VPKEVGPTNADPVCLAKMYYSAVDPT |
| | | | | KDIFTGLIGPMKICKKGSLHANGRQKD |
| | | | | VDKEFYLFPTVFDENESLLLEDNIRMFT |
| | | | | TAPDQVDKEDEDFQESNKMHSMNGFM |
| | | | | YGNQPGLTMCKGDSVVWYLFSAGNEA |
| | | | | DVHGIYFSGNTYLWRGERRDTANLFPQ |
| | | | | TSLTLHMWPDTEGTFNVECLTTDHYTG |
| | | | | GMKQKYTVNQCRRQSEDSTFYLGERT |
| | | | | YYIAAVEVEWDYSPQREWEKELHHLQ |
| | | | | EQNVSNAFLDKGEFYIGSKYKKVVYRQ |
| | | | | YTDSTFRVPVERKAEEEHLGILGPQLHA |
| | | | | DVGDKVKIIFKNMATRPYSIHAHGVQT |
| | | | | ESSTVTPTLPGETLTYVWKIPERSGAGT |
| | | | | EDSACIPWAYYSTVDQVKDLYSGLIGP |
| | | | | LIVCRRPYLKVFNPRRKLEFALLFLVFD |
| | | | | ENESWYLDDNIKTYSDHPEKVNKDDEE |
| | | | | FIESNKMHAINGRMFGNLQGLTMHVG |
| | | | | DEVNWYLMGMGNEIDLHTVHFHGHSF |
| | | | | QYKHRGVYSSDVFDIFPGTYQTLEMFP |
| | | | | RTPGIWLLHCHVTDHIHAGMETTYTVL |
| | | | | QNEDTKSG |
| 301 | IGG1 | Immunoglobulin | P01857 | ASTKGPSVFPLAPSSKSTSGGTAALGCL |
| | | heavy constant | | VKDYFPEPVTVSWNSGALTSGVHTFPA |
| | | gamma 1 | | VLQSSGLYSLSSVVTVPSSSLGTQTYIC |
| | | | | NVNHKPSNTKVDKKVEPKSCDKTHTCP |
| | | | | PCPAPELLGGPSVFLFPPKPKDTLMISRT |
| | | | | PEVTCVVVDVSHEDPEVKFNWYVDGV |
| | | | | EVHNAKTKPREEQYNSTYRVVSVLTVL |
| | | | | HQDWLNGKEYKCKVSNKALPAPIEKTI |
| | | | | SKAKGQPREPQVYTLPPSRDELTKNQV |
| | | | | SLTCLVKGFYPSDIAVEWESNGQPENN |
| | | | | YKTTPPVLDSDGSFFLYSKLTVDKSRW |
| | | | | QQGNVFSCSVMHEALHNHYTQKSLSLS |
| | | | | PGK |
| 302 | HEMO | Hemopexin | P02790 | MARVLGAPVALGLWSLCWSLAIATPLP |
| | | | | PTSAHGNVAEGETKPDPDVTERCSDGW |
| | | | | SFDATTLDDNGTMLFFKGEFVWKSHK |
| | | | | WDRELISERWKNFPSPVDAAFRQGHNS |
| | | | | VFLIKGDKVWVYPPEKKEKGYPKLLQD |
| | | | | EFPGIPSPLDAAVECHRGECQAEGVLFF |
| | | | | QGDREWFWDLATGTMKERSWPAVGN |
| | | | | CSSALRWLGRYYCFQGNQFLRFDPVRG |
| | | | | EVPPRYPRDVRDYFMPCPGRGHGHRN |
| | | | | GTGHGNSTHHGPEYMRCSPHLVLSALT |
| | | | | SDNHGATYAFSGTHYWRLDTSRDGWH |
| | | | | SWPIAHQWPQGPSAVDAAFSWEEKLYL |
| | | | | VQGTQVYVFLTKGGYTLVSGYPKRLEK |
| | | | | EVGTPHGIILDSVDAAFICPGSSRLHIMA |
| | | | | GRRLWWLDLKSGAQATWTELPWPHEK |
| | | | | VDGALCMEKSLGPNSCSANGPGLYLIH |
| | | | | GPNLYCYSDVEKLNAAKALPQPQNVTS |
| | | | | LLGCTH |
| 303 | APOB | Apolipoprotein | P04114 | MDPPRPALLALLALPALLLLLLAGARA |
| | | B-100 | | EEEMLENVSLVCPKDATRFKHLRKYTY |
| | | | | NYEAESSSGVPGTADSRSATRINCKVEL |
| | | | | EVPQLCSFILKTSQCTLKEVYGFNPEGK |
| | | | | ALLKKTKNSEEFAAAMSRYELKLAIPE |
| | | | | GKQVFLYPEKDEPTYILNIKRGIISALLV |
| | | | | PPETEEAKQVLFLDTVYGNCSTHFTVK |
| | | | | TRKGNVATEISTERDLGQCDRFKPIRTG |
| | | | | ISPLALIKGMTRPLSTLISSSQSCQYTLD |
| | | | | AKRKHVAEAICKEQHLFLPFSYKNKYG |
| | | | | MVAQVTQTLKLEDTPKINSRFFGEGTK |
| | | | | KMGLAFESTKSTSPPKQAEAVLKTLQE |
| | | | | LKKLTISEQNIQRANLFNKLVTELRGLS |
| | | | | DEAVTSLLPQLIEVSSPITLQALVQCGQP |
| | | | | QCSTHILQWLKRVHANPLLIDVVTYLV |
| | | | | ALIPEPSAQQLREIFNMARDQRSRATLY |
| | | | | ALSHAVNNYHKTNPTGTQELLDIANYL |
| | | | | MEQIQDDCTGDEDYTYLILRVIGNMGQ |
| | | | | TMEQLTPELKSSILKCVQSTKPSLMIQK |
| | | | | AAIQALRKMEPKDKDQEVLLQTFLDDA |
| | | | | SPGDKRLAAYLMLMRSPSQADINKIVQI |
| | | | | LPWEQNEQVKNFVASHIANILNSEELDI |
| | | | | QDLKKLVKEALKESQLPTVMDFRKFSR |
| | | | | NYQLYKSVSLPSLDPASAKIEGNLIFDP |
| | | | | NNYLPKESMLKTTLTAFGFASADLIEIG |
| | | | | LEGKGFEPTLEALFGKQGFFPDSVNKAL |
| | | | | YWVNGQVPDGVSKVLVDHFGYTKDD |
| | | | | KHEQDMVNGIMLSVEKLIKDLKSKEVP |
| | | | | EARAYLRILGEELGFASLHDLQLLGKLL |
| | | | | LMGARTLQGIPQMIGEVIRKGSKNDFFL |
| | | | | HYIFMENAFELPTGAGLQLQISSSGVIAP |
| | | | | GAKAGVKLEVANMQAELVAKPSVSVE |
| | | | | FVTNMGIIIPDFARSGVQMNTNFFHESG |
| | | | | LEAHVALKAGKLKFIIPSPKRPVKLLSG |
| | | | | GNTLHLVSTTKTEVIPPLIENRQSWSVC |
| | | | | KQVFPGLNYCTSGAYSNASSTDSASYY |
| | | | | PLTGDTRLELELRPTGEIEQYSVSATYE |
| | | | | LQREDRALVDTLKFVTQAEGAKQTEAT |
| | | | | MTFKYNRQSMTLSSEVQIPDFDVDLGTI |
| | | | | LRVNDESTEGKTSYRLTLDIQNKKITEV |
| | | | | ALMGHLSCDTKEERKIKGVISIPRLQAE |
| | | | | ARSEILAHWSPAKLLLQMDSSATAYGS |
| | | | | TVSKRVAWHYDEEKIEFEWNTGTNVD |
| | | | | TKKMTSNFPVDLSDYPKSLHMYANRLL |
| | | | | DHRVPQTDMTFRHVGSKLIVAMSSWL |
| | | | | QKASGSLPYTQTLQDHLNSLKEFNLQN |
| | | | | MGLPDFHIPENLFLKSDGRVKYTLNKN |
| | | | | SLKIEIPLPFGGKSSRDLKMLETVRTPAL |
| | | | | HFKSVGFHLPSREFQVPTFTIPKLYQLQ |
| | | | | VPLLGVLDLSTNVYSNLYNWSASYSGG |
| | | | | NTSTDHFSLRARYHMKADSVVDLLSYN |
| | | | | VQGSGETTYDHKNTFTLSYDGSLRHKF |
| | | | | LDSNIKFSHVEKLGNNPVSKGLLIFDAS |
| | | | | SSWGPQMSASVHLDSKKKQHLFVKEV |
| | | | | KIDGQFRVSSFYAKGTYGLSCQRDPNT |
| | | | | GRLNGESNLRFNSSYLQGTNQITGRYE |
| | | | | DGTLSLTSTSDLQSGIIKNTASLKYENY |
| | | | | ELTLKSDTNGKYKNFATSNKMDMTFS |
| | | | | KQNALLRSEYQADYESLRFFSLLSGSLN |
| | | | | SHGLELNADILGTDKINSGAHKATLRIG |
| | | | | QDGISTSATTNLKCSLLVLENELNAELG |
| | | | | LSGASMKLTTNGRFREHNAKFSLDGKA |
| | | | | ALTELSLGSAYQAMILGVDSKNIFNFKV |
| | | | | SQEGLKLSNDMMGSYAEMKFDHTNSL |
| | | | | NIAGLSLDFSSKLDNIYSSDKFYKQTVN |
| | | | | LQLQPYSLVTTLNSDLKYNALDLTNNG |
| | | | | KLRLEPLKLHVAGNLKGAYQNNEIKHI |
| | | | | YAISSAALSASYKADTVAKVQGVEFSH |
| | | | | RLNTDIAGLASAIDMSTNYNSDSLHFSN |
| | | | | VFRSVMAPFTMTIDAHTNGNGKLALW |
| | | | | GEHTGQLYSKFLLKAEPLAFTFSHDYK |
| | | | | GSTSHHLVSRKSISAALEHKVSALLTPA |
| | | | | EQTGTWKLKTQFNNNEYSQDLDAYNT |
| | | | | KDKIGVELTGRTLADLTLLDSPIKVPLL |
| | | | | LSEPINIIDALEMRDAVEKPQEFTIVAFV |
| | | | | KYDKNQDVHSINLPFFETLQEYFERNR |
| | | | | QTIIVVLENVQRNLKHINIDQFVRKYRA |
| | | | | ALGKLPQQANDYLNSFNWERQVSHAK |
| | | | | EKLTALTKKYRITENDIQIALDDAKINF |
| | | | | NEKLSQLQTYMIQFDQYIKDSYDLHDL |
| | | | | KIAIANIIDEIIEKLKSLDEHYHIRVNLVK |
| | | | | TIHDLHLFIENIDFNKSGSSTASWIQNVD |
| | | | | TKYQIRIQIQEKLQQLKRHIQNIDIQHLA |
| | | | | GKLKQHIEAIDVRVLLDQLGTTISFERIN |
| | | | | DILEHVKHFVINLIGDFEVAEKINAFRA |
| | | | | KVHELIERYEVDQQIQVLMDKLVELAH |
| | | | | QYKLKETIQKLSNVLQQVKIKDYFEKL |
| | | | | VGFIDDAVKKLNELSFKTFIEDVNKFLD |
| | | | | MLIKKLKSFDYHQFVDETNDKIREVTQ |
| | | | | RLNGEIQALELPQKAEALKLFLEETKAT |
| | | | | VAVYLESLQDTKITLIINWLQEALSSAS |
| | | | | LAHMKAKFRETLEDTRDRMYQMDIQQ |
| | | | | ELQRYLSLVGQVYSTLVTYISDWWTLA |
| | | | | AKNLTDFAEQYSIQDWAKRMKALVEQ |
| | | | | GFTVPEIKTILGTMPAFEVSLQALQKAT |
| | | | | FQTPDFIVPLTDLRIPSVQINFKDLKNIKI |
| | | | | PSRFSTPEFTILNTFHIPSFTIDFVEMKVK |
| | | | | IIRTIDQMLNSELQWPVPDIYLRDLKVE |
| | | | | DIPLARITLPDFRLPEIAIPEFIIPTLNLND |
| | | | | FQVPDLHIPEFQLPHISHTIEVPTFGKLY |
| | | | | SILKIQSPLFTLDANADIGNGTTSANEAG |
| | | | | IAASITAKGESKLEVLNFDFQANAQLSN |
| | | | | PKINPLALKESVKFSSKYLRTEHGSEML |
| | | | | FFGNAIEGKSNTVASLHTEKNTLELSNG |
| | | | | VIVKINNQLTLDSNTKYFHKLNIPKLDF |
| | | | | SSQADLRNEIKTLLKAGHIAWTSSGKGS |
| | | | | WKWACPRFSDEGTHESQISFTIEGPLTS |
| | | | | FGLSNKINSKHLRVNQNLVYESGSLNFS |
| | | | | KLEIQSQVDSQHVGHSVLTAKGMALFG |
| | | | | EGKAEFTGRHDAHLNGKVIGTLKNSLF |
| | | | | FSAQPFEITASTNNEGNLKVRFPLRLTG |
| | | | | KIDFLNNYALFLSPSAQQASWQVSARF |
| | | | | NQYKYNQNFSAGNNENIMEAHVGING |
| | | | | EANLDFLNIPLTIPEMRLPYTIITTPPLKD |
| | | | | FSLWEKTGLKEFLKTTKQSFDLSVKAQ |
| | | | | YKKNKHRHSITNPLAVLCEFISQSIKSFD |
| | | | | RHFEKNRNNALDFVTKSYNETKIKFDK |
| | | | | YKAEKSHDELPRTFQIPGYTVPVVNVE |
| | | | | VSPFTIEMSAFGYVFPKAVSMPSFSILGS |
| | | | | DVRVPSYTLILPSLELPVLHVPRNLKLSL |
| | | | | PDFKELCTISHIFIPAMGNITYDFSFKSSV |
| | | | | ITLNTNAELFNQSDIVAHLLSSSSSVIDA |
| | | | | LQYKLEGTTRLTRKRGLKLATALSLSN |
| | | | | KFVEGSHNSTVSLTTKNMEVSVATTTK |
| | | | | AQIPILRMNFKQELNGNTKSKPTVSSSM |
| | | | | EFKYDFNSSMLYSTAKGAVDHKLSLES |
| | | | | LTSYFSIESSTKGDVKGSVLSREYSGTIA |
| | | | | SEANTYLNSKSTRSSVKLQGTSKIDDIW |
| | | | | NLEVKENFAGEATLQRIYSLWEHSTKN |
| | | | | HLQLEGLFFTNGEHTSKATLELSPWQM |
| | | | | SALVQVHASQPSSFHDFPDLGQEVALN |
| | | | | ANTKNQKIRWKNEVRIHSGSFQSQVEL |
| | | | | SNDQEKAHLDIAGSLEGHLRFLKNIILP |
| | | | | VYDKSLWDFLKLDVTTSIGRRQHLRVS |
| | | | | TAFVYTKNPNGYSFSIPVKVLADKFIIPG |
| | | | | LKLNDLNSVLVMPTFHVPFTDLQVPSC |
| | | | | KLDFREIQIYKKLRTSSFALNLPTLPEVK |
| | | | | FPEVDVLTKYSQPEDSLIPFFEITVPESQ |
| | | | | LTVSQFTLPKSVSDGIAALDLNAVANKI |
| | | | | ADFELPTIIVPEQTIEIPSIKFSVPAGIVIPS |
| | | | | FQALTARFEVDSPVYNATWSASLKNKA |
| | | | | DYVETVLDSTCSSTVQFLEYELNVLGT |
| | | | | HKIEDGTLASKTKGTFAHRDFSAEYEE |
| | | | | DGKYEGLQEWEGKAHLNIKSPAFTDLH |
| | | | | LRYQKDKKGISTSAASPAVGTVGMDM |
| | | | | DEDDDFSKWNFYYSPQSSPDKKLTIFKT |
| | | | | ELRVRESDEETQIKVNWEEEAASGLLTS |
| | | | | LKDNVPKATGVLYDYVNKYHWEHTGL |
| | | | | TLREVSSKLRRNLQNNAEWVYQGAIRQ |
| | | | | IDDIDVRFQKAASGTTGTYQEWKDKAQ |
| | | | | NLYQELLTQEGQASFQGLKDNVFDGLV |
| | | | | RVTQEFHMKVKHLIDSLIDFLNFPRFQF |
| | | | | PGKPGIYTREELCTMFIREVGTVLSQVY |
| | | | | SKVHNGSEILFSYFQDLVITLPFELRKHK |
| | | | | LIDVISMYRELLKDLSKEAQEVFKAIQS |
| | | | | LKTTEVLRNLQDLLQFIFQLIEDNIKQLK |
| | | | | EMKFTYLINYIQDEINTIFSDYIPYVFKL |
| | | | | LKENLCLNLHKFNEFIQNELQEASQELQ |
| | | | | QIHQYIMALREEYFDPSIVGWTVKYYE |
| | | | | LEEKIVSLIKNLLVALKDFHSEYIVSASN |
| | | | | FTSQLSSQVEQFLHRNIQEYLSILTDPDG |
| | | | | KGKEKIAELSATAQEIIKSQAIATKKIISD |
| | | | | YHQQFRYKLQDFSDQLSDYYEKFIAES |
| | | | | KRLIDLSIQNYHTFLIYITELLKKLQSTT |
| | | | | VMNPYMKLAPGELTIIL |
| 304 | HPT | Haptoglobin | P00738 | MSALGAVIALLLWGQLFAVDSGNDVT |
| | | | | DIADDGCPKPPEIAHGYVEHSVRYQCK |
| | | | | NYYKLRTEGDGVYTLNDKKQWINKAV |
| | | | | GDKLPECEADDGCPKPPEIAHGYVEHS |
| | | | | VRYQCKNYYKLRTEGDGVYTLNNEKQ |
| | | | | WINKAVGDKLPECEAVCGKPKNPANP |
| | | | | VQRILGGHLDAKGSFPWQAKMVSHHN |
| | | | | LTTGATLINEQWLLTTAKNLFLNHSEN |
| | | | | ATAKDIAPTLTLYVGKKQLVEIEKVVL |
| | | | | HPNYSQVDIGLIKLKQKVSVNERVMPIC |
| | | | | LPSKDYAEVGRVGYVSGWGRNANFKF |
| | | | | TDHLKYVMLPVADQDQCIRHYEGSTVP |
| | | | | EKKTPKSPVGVQPILNEHTFCAGMSKY |
| | | | | QEDTCYGDAGSAFAVHDLEEDTWYAT |
| | | | | GILSFDKSCAVAEYGVYVKVTSIQDWV |
| | | | | QKTIAEN |
| 305 | IGG3 | Immunoglobulin | P01860 | ASTKGPSVFPLAPCSRSTSGGTAALGCL |
| | | heavy constant | | VKDYFPEPVTVSWNSGALTSGVHTFPA |
| | | gamma 3 | | VLQSSGLYSLSSVVTVPSSSLGTQTYTC |
| | | | | NVNHKPSNTKVDKRVELKTPLGDTTHT |
| | | | | CPRCPEPKSCDTPPPCPRCPEPKSCDTPP |
| | | | | PCPRCPEPKSCDTPPPCPRCPAPELLGGP |
| | | | | SVFLFPPKPKDTLMISRTPEVTCVVVDV |
| | | | | SHEDPEVQFKWYVDGVEVHNAKTKPR |
| | | | | EEQYNSTFRVVSVLTVLHQDWLNGKE |
| | | | | YKCKVSNKALPAPIEKTISKTKGQPREP |
| | | | | QVYTLPPSREEMTKNQVSLTCLVKGFY |
| | | | | PSDIAVEWESSGQPENNYNTTPPMLDS |
| | | | | DGSFFLYSKLTVDKSRWQQGNIFSCSV |
| | | | | MHEALHNRFTQKSLSLSPGK |
| 306 | IGG34 | Immunoglobulin | P01860 | ASTKGPSVFPLAPCSRSTSGGTAALGCL |
| | | heavy constant | | VKDYFPEPVTVSWNSGALTSGVHTFPA |
| | | gamma 34 | | VLQSSGLYSLSSVVTVPSSSLGTQTYTC |
| | | | | NVNHKPSNTKVDKRVELKTPLGDTTHT |
| | | | | CPRCPEPKSCDTPPPCPRCPEPKSCDTPP |
| | | | | PCPRCPEPKSCDTPPPCPRCPAPELLGGP |
| | | | | SVFLFPPKPKDTLMISRTPEVTCVVVDV |
| | | | | SHEDPEVQFKWYVDGVEVHNAKTKPR |
| | | | | EEQYNSTFRVVSVLTVLHQDWLNGKE |
| | | | | YKCKVSNKALPAPIEKTISKTKGQPREP |
| | | | | QVYTLPPSREEMTKNQVSLTCLVKGFY |
| | | | | PSDIAVEWESSGQPENNYNTTPPMLDS |
| | | | | DGSFFLYSKLTVDKSRWQQGNIFSCSV |
| | | | | MHEALHNRFTQKSLSLSPGK |
| 417 | CO2 | ComplementC | P06681 | MGPLMVLFCLLFLYPGLADSAPSCPQN |
| | | 2 | | VNISGGTFTLSHGWAPGSLLTYSCPQGL |
| | | | | YPSPASRLCKSSGQWQTPGATRSLSKA |
| | | | | VCKPVRCPAPVSFENGIYTPRLGSYPVG |
| | | | | GNVSFECEDGFILRGSPVRQCRPNGMW |
| | | | | DGETAVCDNGAGHCPNPGISLGAVRTG |
| | | | | FRFGHGDKVRYRCSSNLVLTGSSEREC |
| | | | | QGNGVWSGTEPICRQPYSYDFPEDVAP |
| | | | | ALGTSFSHMLGATNPTQKTKESLGRKI |
| | | | | QIQRSGHLNLYLLLDCSQSVSENDFLIF |
| | | | | KESASLMVDRIFSFEINVSVAIITFASEPK |
| | | | | VLMSVLNDNSRDMTEVISSLENANYKD |
| | | | | HENGTGTNTYAALNSVYLMMNNQMR |
| | | | | LLGMETMAWQEIRHAIILLTDGKSNMG |
| | | | | GSPKTAVDHIREILNINQKRNDYLDIYAI |
| | | | | GVGKLDVDWRELNELGSKKDGERHAF |
| | | | | ILQDTKALHQVFEHMLDVSKLTDTICG |
| | | | | VGNMSANASDQERTPWHVTIKPKSQET |
| | | | | CRGALISDQWVLTAAHCFRDGNDHSL |
| | | | | WRVNVGDPKSQWGKEFLIEKAVISPGF |
| | | | | DVFAKKNQGILEFYGDDIALLKLAQKV |
| | | | | KMSTHARPICLPCTMEANLALRRPQGS |
| | | | | TCRDHENELLNKQSVPAHFVALNGSKL |
| | | | | NINLKMGVEWTSCAEVVSQEKTMFPNL |
| | | | | TDVREVVTDQFLCSGTQEDESPCKGES |
| | | | | GGAVFLERRFRFFQVGLVSWGLYNPCL |
| | | | | GSADKNSRKRAPRSKVPPPRDFHINLFR |
| | | | | MQPWLRQHLGDVLNFLPL |
| 418 | AGP1 | Alpha-1-acid | P02763 | MALSWVLTVLSLLPLLEAQIPLCANLVP |
| | | glycoprotein 1 | | VPITNATLDQITGKWFYIASAFRNEEYN |
| | | | | KSVQEIQATFFYFTPNKTEDTIFLREYQT |
| | | | | RQDQCIYNTTYLNVQRENGTISRYVGG |
| | | | | QEHFAHLLILRDTKTYMLAFDVNDEKN |
| | | | | WGLSVYADKPETTKEQLGEFYEALDCL |
| | | | | RIPKSDVVYTDWKKDKCEPLEKQHEKE |
| | | | | RKQEEGES |
| 419 | AACT | Alpha-1- | P01011 | MERMLPLLALGLLAAGFCPAVLCHPNS |
| | | anti- | | PLDEENLTQENQDRGTHVDLGLASANV |
| | | chymotrypsin | | DFAFSLYKQLVLKAPDKNVIFSPLSISTA |
| | | | | LAFLSLGAHNTTLTEILKGLKFNLTETS |
| | | | | EAEIHQSFQHLLRTLNQSSDELQLSMGN |
| | | | | AMFVKEQLSLLDRFTEDAKRLYGSEAF |
| | | | | ATDFQDSAAAKKLINDYVKNGTRGKIT |
| | | | | DLIKDLDSQTMMVLVNYIFFKAKWEM |
| | | | | PFDPQDTHQSRFYLSKKKWVMVPMMS |
| | | | | LHHLTIPYFRDEELSCTVVELKYTGNAS |
| | | | | ALFILPDQDKMEEVEAMLLPETLKRWR |
| | | | | DSLEFREIGELYLPKFSISRDYNLNDILL |
| | | | | QLGIEEAFTSKADLSGITGARNLAVSQV |
| | | | | VHKAVLDVFEEGTEASAATAVKITLLS |
| | | | | ALVETRTIVRFNRPFLMIIVPTDTQNIFF |
| | | | | MSKVTNPKQA |
| 420 | A2MG | Alpha-2- | P01023 | MGKNKLLHPSLVLLLLVLLPTDASVSG |
| | | macroglobulin | | KPQYMVLVPSLLHTETTEKGCVLLSYL |
| | | | | NETVTVSASLESVRGNRSLFTDLEAEND |
| | | | | VLHCVAFAVPKSSSNEEVMFLTVQVKG |
| | | | | PTQEFKKRTTVMVKNEDSLVFVQTDKS |
| | | | | IYKPGQTVKFRVVSMDENFHPLNELIPL |
| | | | | VYIQDPKGNRIAQWQSFQLEGGLKQFS |
| | | | | FPLSSEPFQGSYKVVVQKKSGGRTEHPF |
| | | | | TVEEFVLPKFEVQVTVPKIITILEEEMNV |
| | | | | SVCGLYTYGKPVPGHVTVSICRKYSDA |
| | | | | SDCHGEDSQAFCEKFSGQLNSHGCFYQ |
| | | | | QVKTKVFQLKRKEYEMKLHTEAQIQEE |
| | | | | GTVVELTGRQSSEITRTITKLSFVKVDS |
| | | | | HFRQGIPFFGQVRLVDGKGVPIPNKVIFI |
| | | | | RGNEANYYSNATTDEHGLVQFSINTTN |
| | | | | VMGTSLTVRVNYKDRSPCYGYQWVSE |
| | | | | EHEEAHHTAYLVFSPSKSFVHLEPMSHE |
| | | | | LPCGHTQTVQAHYILNGGTLLGLKKLS |
| | | | | FYYLIMAKGGIVRTGTHGLLVKQEDMK |
| | | | | GHFSISIPVKSDIAPVARLLIYAVLPTGD |
| | | | | VIGDSAKYDVENCLANKVDLSFSPSQSL |
| | | | | PASHAHLRVTAAPQSVCALRAVDQSVL |
| | | | | LMKPDAELSASSVYNLLPEKDLTGFPGP |
| | | | | LNDQDNEDCINRHNVYINGITYTPVSST |
| | | | | NEKDMYSFLEDMGLKAFTNSKIRKPKM |
| | | | | CPQLQQYEMHGPEGLRVGFYESDVMG |
| | | | | RGHARLVHVEEPHTETVRKYFPETWIW |
| | | | | DLVVVNSAGVAEVGVTVPDTITEWKA |
| | | | | GAFCLSEDAGLGISSTASLRAFQPFFVEL |
| | | | | TMPYSVIRGEAFTLKATVLNYLPKCIRV |
| | | | | SVQLEASPAFLAVPVEKEQAPHCICANG |
| | | | | RQTVSWAVTPKSLGNVNFTVSAEALES |
| | | | | QELCGTEVPSVPEHGRKDTVIKPLLVEP |
| | | | | EGLEKETTFNSLLCPSGGEVSEELSLKLP |
| | | | | PNVVEESARASVSVLGDILGSAMQNTQ |
| | | | | NLLQMPYGCGEQNMVLFAPNIYVLDY |
| | | | | LNETQQLTPEIKSKAIGYLNTGYQRQLN |
| | | | | YKHYDGSYSTFGERYGRNQGNTWLTA |
| | | | | FVLKTFAQARAYIFIDEAHITQALIWLS |
| | | | | QRQKDNGCFRSSGSLLNNAIKGGVEDE |
| | | | | VTLSAYITIALLEIPLTVTHPVVRNALFC |
| | | | | LESAWKTAQEGDHGSHVYTKALLAYA |
| | | | | FALAGNQDKRKEVLKSLNEEAVKKDN |
| | | | | SVHWERPQKPKAPVGHFYEPQAPSAEV |
| | | | | EMTSYVLLAYLTAQPAPTSEDLTSATNI |
| | | | | VKWITKQQNAQGGFSSTQDTVVALHA |
| | | | | LSKYGAATFTRTGKAAQVTIQSSGTFSS |
| | | | | KFQVDNNNRLLLQQVSLPELPGEYSMK |
| | | | | VTGEGCVYLQTSLKYNILPEKEEFPFAL |
| | | | | GVQTLPQTCDEPKAHTSFQISLSVSYTG |
| | | | | SRSASNMAIVDVKMVSGFIPLKPTVKM |
| | | | | LERSNHVSRTEVSSNHVLIYLDKVSNQT |
| | | | | LSLFFTVLQDVPVRDLKPAIVKVYDYY |
| | | | | ETDEFAIAEYNAPCSKDLGNA |
| 421 | A1AT | Alpha-1- | P01009 | MPSSVSWGILLLAGLCCLVPVSLAEDPQ |
| | | antitrypsin | | GDAAQKTDTSHHDQDHPTFNKITPNLA |
| | | | | EFAFSLYRQLAHQSNSTNIFFSPVSIATA |
| | | | | FAMLSLGTKADTHDEILEGLNFNLTEIP |
| | | | | EAQIHEGFQELLRTLNQPDSQLQLTTGN |
| | | | | GLFLSEGLKLVDKFLEDVKKLYHSEAF |
| | | | | TVNFGDTEEAKKQINDYVEKGTQGKIV |
| | | | | DLVKELDRDTVFALVNYIFFKGKWERP |
| | | | | FEVKDTEEEDFHVDQVTTVKVPMMKR |
| | | | | LGMFNIQHCKKLSSWVLLMKYLGNAT |
| | | | | AIFFLPDEGKLQHLENELTHDIITKFLEN |
| | | | | EDRRSASLHLPKLSITGTYDLKSVLGQL |
| | | | | GITKVFSNGADLSGVTEEAPLKLSKAV |
| | | | | HKAVLTIDEKGTEAAGAMFLEAIPMSIP |
| | | | | PEVKFNKPFVFLMIEQNTKSPLFMGKV |
| | | | | VNPTQK |
| 422 | VTNC | Vitronectin | P04004 | MAPLRPLLILALLAWVALADQESCKGR |
| | | | | CTEGFNVDKKCQCDELCSYYQSCCTDY |
| | | | | TAECKPQVTRGDVFTMPEDEYTVYDD |
| | | | | GEEKNNATVHEQVGGPSLTSDLQAQSK |
| | | | | GNPEQTPVLKPEEEAPAPEVGASKPEGI |
| | | | | DSRPETLHPGRPQPPAEEELCSGKPFDA |
| | | | | FTDLKNGSLFAFRGQYCYELDEKAVRP |
| | | | | GYPKLIRDVWGIEGPIDAAFTRINCQGK |
| | | | | TYLFKGSQYWRFEDGVLDPDYPRNISD |
| | | | | GFDGIPDNVDAALALPAHSYSGRERVY |
| | | | | FFKGKQYWEYQFQHQPSQEECEGSSLS |
| | | | | AVFEHFAMMQRDSWEDIFELLFWGRTS |
| | | | | AGTRQPQFISRDWHGVPGQVDAAMAG |
| | | | | RIYISGMAPRPSLAKKQRFRHRNRKGY |
| | | | | RSQRGHSRGRNQNSRRPSRATWLSLFS |
| | | | | SEESNLGANNYDDYRMDWLVPATCEPI |
| | | | | QSVFFFSGDKYYRVNLRTRRVDTVDPP |
| | | | | YPRSIAQYWLGCPAPGHL |
| 423 | IGG2 | Immunoglobulin | P01859 | ASTKGPSVFPLAPCSRSTSESTAALGCL |
| | | heavy constant | | VKDYFPEPVTVSWNSGALTSGVHTFPA |
| | | gamma 2 | | VLQSSGLYSLSSVVTVPSSNFGTQTYTC |
| | | | | NVDHKPSNTKVDKTVERKCCVECPPCP |
| | | | | APPVAGPSVFLFPPKPKDTLMISRTPEVT |
| | | | | CVVVDVSHEDPEVQFNWYVDGVEVHN |
| | | | | AKTKPREEQFNSTFRVVSVLTVVHQDW |
| | | | | LNGKEYKCKVSNKGLPAPIEKTISKTKG |
| | | | | QPREPQVYTLPPSREEMTKNQVSLTCL |
| | | | | VKGFYPSDISVEWESNGQPENNYKTTPP |
| | | | | MLDSDGSFFLYSKLTVDKSRWQQGNV |
| | | | | FSCSVMHEALHNHYTQKSLSLSPGK |
| | | | | MRLLAKIICLMLWAICVAEDCNELPPRR |
| | | | | NTEILTGSWSDQTYPEGTQAIYKCRPGY |
| | | | | RSLGNVIMVCRKGEWVALNPLRKCQK |
| | | | | RPCGHPGDTPFGTFTLTGGNVFEYGVK |
| | | | | AVYTCNEGYQLLGEINYRECDTDGWT |
| | | | | NDIPICEVVKCLPVTAPENGKIVSSAME |
| | | | | PDREYHFGQAVRFVCNSGYKIEGDEEM |
| | | | | HCSDDGFWSKEKPKCVEISCKSPDVING |
| 424 | CFAH | Complement | P08603 | SPISQKIIYKENERFQYKCNMGYEYSER |
| | | Factor H | | GDAVCTESGWRPLPSCEEKSCDNPYIPN |
| | | | | GDYSPLRIKHRTGDEITYQCRNGFYPAT |
| | | | | RGNTAKCTSTGWIPAPRCTLKPCDYPDI |
| | | | | KHGGLYHENMRRPYFPVAVGKYYSYY |
| | | | | CDEHFETPSGSYWDHIHCTQDGWSPAV |
| | | | | PCLRKCYFPYLENGYNQNYGRKFVQG |
| | | | | KSIDVACHPGYALPKAQTTVTCMENG |
| | | | | WSPTPRCIRVKTCSKSSIDIENGFISESQY |
| | | | | TYALKEKAKYQCKLGYVTADGETSGSI |
| | | | | TCGKDGWSAQPTCIKSCDIPVFMNART |
| | | | | KNDFTWFKLNDTLDYECHDGYESNTG |
| | | | | STTGSIVCGYNGWSDLPICYERECELPKI |
| | | | | DVHLVPDRKKDQYKVGEVLKFSCKPG |
| | | | | FTIVGPNSVQCYHFGLSPDLPICKEQVQ |
| | | | | SCGPPPELLNGNVKEKTKEEYGHSEVV |
| | | | | EYYCNPRFLMKGPNKIQCVDGEWTTLP |
| | | | | VCIVEESTCGDIPELEHGWAQLSSPPYY |
| | | | | YGDSVEFNCSESFTMIGHRSITCIHGVW |
| | | | | TQLPQCVAIDKLKKCKSSNLIILEEHLK |
| | | | | NKKEFDHNSNIRYRCRGKEGWIHTVCI |
| | | | | NGRWDPEVNCSMAQIQLCPPPPQIPNSH |
| | | | | NMTTTLNYRDGEKVSVLCQENYLIQEG |
| | | | | EEITCKDGRWQSIPLCVEKIPCSQPPQIE |
| | | | | HGTINSSRSSQESYAHGTKLSYTCEGGF |
| | | | | RISEENETTCYMGKWSSPPQCEGLPCKS |
| | | | | PPEISHGVVAHMSDSYQYGEEVTYKCF |
| | | | | EGFGIDGPAIAKCLGEKWSHPPSCIKTD |
| | | | | CLSLPSFENAIPMGEKKDVYKAGEQVT |
| | | | | YTCATYYKMDGASNVTCINSRWTGRP |
| | | | | TCRDTSCVNPPTVQNAYIVSRQMSKYP |
| | | | | SGERVRYQCRSPYEMFGDEEVMCLNG |
| | | | | NWTEPPQCKDSTGKCGPPPPIDNGDITS |
| | | | | FPLSVYAPASSVEYQCQNLYQLEGNKRI |
| | | | | TCRNGQWSEPPKCLHPCVISREIMENYN |
| | | | | IALRWTAKQKLYSRTGESVEFVCKRGY |
| | | | | RLSSRSHTLRTTCWDGKLEYPTCAKR |
| 425 | APOH | Beta-2- | P02749 | MISPVLILFSSFLCHVAIAGRTCPKPDDL |
| | | glycoprotein 1 | | PFSTVVPLKTFYEPGEEITYSCKPGYVSR |
| | | | | GGMRKFICPLTGLWPINTLKCTPRVCPF |
| | | | | AGILENGAVRYTTFEYPNTISFSCNTGF |
| | | | | YLNGADSAKCTEEGKWSPELPVCAPIIC |
| | | | | PPPSIPTFATLRVYKPSAGNNSLYRDTA |
| | | | | VFECLPQHAMFGNDTITCTTHGNWTKL |
| | | | | PECREVKCPFPSRPDNGFVNYPAKPTLY |
| | | | | YKDKATFGCHDGYSLDGPEEIECTKLG |
| | | | | NWSAMPSCKASCKVPVKKATVVYQGE |
| | | | | RVKIQEKFKNGMLHGDKVSFFCKNKEK |
| | | | | KCSYTEDAQCIDGTIEVPKCFKEHSSLA |
| | | | | FWKTDASDVKPC |
| 426 | APOD | Apolipoprotein | P05090 | MVMLLLLLSALAGLFGAAEGQAFHLG |
| | | D | | KCPNPPVQENFDVNKYLGRWYEIEKIPT |
| | | | | TFENGRCIQANYSLMENGKIKVLNQEL |
| | | | | RADGTVNQIEGEATPVNLTEPAKLEVK |
| | | | | FSWFMPSAPYWILATDYENYALVYSCT |
| | | | | CIIQLFHVDFAWILARNPNLPPETVDSL |
| | | | | KNILTSNNIDVKKMTVTDQVNCPKLS |
| 427 | TRFE | Serotransferrin | P02787 | MRLAVGALLVCAVLGLCLAVPDKTVR |
| | | | | WCAVSEHEATKCQSFRDHMKSVIPSDG |
| | | | | PSVACVKKASYLDCIRAIAANEADAVT |
| | | | | LDAGLVYDAYLAPNNLKPVVAEFYGS |
| | | | | KEDPQTFYYAVAVVKKDSGFQMNQLR |
| | | | | GKKSCHTGLGRSAGWNIPIGLLYCDLPE |
| | | | | PRKPLEKAVANFFSGSCAPCADGTDFP |
| | | | | QLCQLCPGCGCSTLNQYFGYSGAFKCL |
| | | | | KDGAGDVAFVKHSTIFENLANKADRD |
| | | | | QYELLCLDNTRKPVDEYKDCHLAQVPS |
| | | | | HTVVARSMGGKEDLIWELLNQAQEHF |
| | | | | GKDKSKEFQLFSSPHGKDLLFKDSAHG |
| | | | | FLKVPPRMDAKMYLGYEYVTAIRNLRE |
| | | | | GTCPEAPTDECKPVKWCALSHHERLKC |
| | | | | DEWSVNSVGKIECVSAETTEDCIAKIMN |
| | | | | GEADAMSLDGGFVYIAGKCGLVPVLAE |
| | | | | NYNKSDNCEDTPEAGYFAVAVVKKSA |
| | | | | SDLTWDNLKGKKSCHTAVGRTAGWNI |
| | | | | PMGLLYNKINHCRFDEFFSEGCAPGSK |
| | | | | KDSSLCKLCMGSGLNLCEPNNKEGYYG |
| | | | | YTGAFRCLVEKGDVAFVKHQTVPQNT |
| | | | | GGKNPDPWAKNLNEKDYELLCLDGTR |
| | | | | KPVEEYANCHLARAPNHAVVTRKDKE |
| | | | | ACVHKILRQQQHLFGSNVTDCSGNFCL |
| | | | | FRSETKDLLFRDDTVCLAKLHDRNTYE |
| | | | | KYLGEEYVKAVGNLRKCSTSSLLEACT |
| | | | | FRRP |
| 444 | FETUA | Alpha-2-HS- | P02765 | MKSLVLLLCLAQLWGCHSAPHGPGLIY |
| | | glycoprotein | | RQPNCDDPETEEAALVAIDYINQNLPW |
| | | | | GYKHTLNQIDEVKVWPQQPSGELFEIEI |
| | | | | DTLETTCHVLDPTPVARCSVRQLKEHA |
| | | | | VEGDCDFQLLKLDGKFSVVYAKCDSSP |
| | | | | DSAEDVRKVCQDCPLLAPLNDTRVVH |
| | | | | AAKAALAAFNAQNNGSNFQLEEISRAQ |
| | | | | LVPLPPSTYVEFTVSGTDCVAKEATEAA |
| | | | | KCNLLAEKQYGFCKATLSEKLGGAEVA |
| | | | | VTCMVFQTQPVSSQPQPEGANEAVPTP |
| | | | | VVDPDAPPSPPLGAPGLPPAGSPPDSHV |
| | | | | LLAAPPGHQLHRAHYDLRHTFMGVVS |
| | | | | LGSPSGEVSHPRKTRTVVQPSVGAAAG |
| | | | | PVVPPCPGRIRHFKV |
| 445 | A2GL | Leucine-rich | P02750 | MSSWSRQRPKSPGGIQPHVSRTLFLLLL |
| | | Alpha-2- | | LAASAWGVTLSPKDCQVFRSDHGSSIS |
| | | glycoprotein | | CQPPAEIPGYLPADTVHLAVEFFNLTHL |
| | | | | PANLLQGASKLQELHLSSNGLESLSPEF |
| | | | | LRPVPQLRVLDLTRNALTGLPPGLFQAS |
| | | | | ATLDTLVLKENQLEVLEVSWLHGLKAL |
| | | | | GHLDLSGNRLRKLPPGLLANFTLLRTLD |
| | | | | LGENQLETLPPDLLRGPLQLERLHLEGN |
| | | | | KLQVLGKDLLLPQPDLRYLFLNGNKLA |
| | | | | RVAAGAFQGLRQLDMLDLSNNSLASVP |
| | | | | EGLWASLGQPNWDMRDGFDISGNPWI |
| | | | | CDQNLSDLYRWLQAQKDKMFSQNDTR |
| | | | | CAGPEAVKGQTLLAVAKSQ |
| 446 | TTR | Transthyretin | P02766 | MASHRLLLLCLAGLVFVSEAGPTGTGE |
| | | | | SKCPLMVKVLDAVRGSPAINVAVHVFR |
| | | | | KAADDTWEPFASGKTSESGELHGLTTE |
| | | | | EEFVEGIYKVEIDTKSYWKALGISPFHE |
| | | | | HAEVVFTANDSGPRRYTIAALLSPYSYS |
| | | | | TTAVVTNPKE |
| 447 | AFAM | Afamin | P43652 | MKLLKLTGFIFFLFFLTESLTLPTQPRDI |
| | | | | ENFNSTQKFIEDNIEYITIIAFAQYVQEA |
| | | | | TFEEMEKLVKDMVEYKDRCMADKTLP |
| | | | | ECSKLPNNVLQEKICAMEGLPQKHNFS |
| | | | | HCCSKVDAQRRLCFFYNKKSDVGFLPP |
| | | | | FPTLDPEEKCQAYESNRESLLNHFLYEV |
| | | | | ARRNPFVFAPTLLTVAVHFEEVAKSCC |
| | | | | EEQNKVNCLQTRAIPVTQYLKAFSSYQ |
| | | | | KHVCGALLKFGTKVVHFIYIAILSQKFP |
| | | | | KIEFKELISLVEDVSSNYDGCCEGDVVQ |
| | | | | CIRDTSKVMNHICSKQDSISSKIKECCEK |
| | | | | KIPERGQCIINSNKDDRPKDLSLREGKFT |
| | | | | DSENVCQERDADPDTFFAKFTFEYSRR |
| | | | | HPDLSIPELLRIVQIYKDLLRNCCNTENP |
| | | | | PGCYRYAEDKFNETTEKSLKMVQQEC |
| | | | | KHFQNLGKDGLKYHYLIRLTKIAPQLST |
| | | | | EELVSLGEKMVTAFTTCCTLSEEFACV |
| | | | | DNLADLVFGELCGVNENRTINPAVDHC |
| | | | | CKTNFAFRRPCFESLKADKTYVPPPFSQ |
| | | | | DLFTFHADMCQSQNEELQRKTDRFLVN |
| | | | | LVKLKHELTDEELQSLFTNFANVVDKC |
| | | | | CKAESPEVCFNEESPKIGN |
| 448 | APOA1 | Apolipoprotein | P02647 | MKAAVLTLAVLFLTGSQARHFWQQDE |
| | | A-I | | PPQSPWDRVKDLATVYVDVLKDSGRD |
| | | C4 b-binding | | YVSQFEGSALGKQLNLKLLDNWDSVTS |
| | | | | TFSKLREQLGPVTQEFWDNLEKETEGL |
| | | | | RQEMSKDLEEVKAKVQPYLDDFQKKW |
| | | | | QEEMELYRQKVEPLRAELQEGARQKL |
| | | | | HELQEKLSPLGEEMRDRARAHVDALRT |
| | | | | HLAPYSDELRQRLAARLEALKENGGAR |
| | | | | LAEYHAKATEHLSTLSEKAKPALEDLR |
| | | | | QGLLPVLESFKVSFLSALEEYTKKLNTQ |
| | | | | MHPPKTPSGALHRKRKMAAWPFSRLW |
| | | | | KVSDPILFQMTLIAALLPAVLGNCGPPP |
| | | | | TLSFAAPMDITLTETRFKTGTTLKYTCL |
| | | | | PGYVRSHSTQTLTCNSDGEWVYNTFCI |
| | | | | YKRCRHPGELRNGQVEIKTDLSFGSQIE |
| | | | | FSCSEGFFLIGSTTSRCEVQDRGVGWSH |
| 449 | C4BPA | protein alpha | P04003 | PLPQCEIVKCKPPPDIRNGRHSGEENFY |
| | | chain | | AYGFSVTYSCDPRFSLLGHASISCTVEN |
| | | | | ETIGVWRPSPPTCEKITCRKPDVSHGEM |
| | | | | VSGFGPIYNYKDTIVFKCQKGFVLRGSS |
| | | | | VIHCDADSKWNPSPPACEPNSCINLPDIP |
| | | | | HASWETYPRPTKEDVYVVGTVLRYRC |
| | | | | HPGYKPTTDEPTTVICQKNLRWTPYQG |
| | | | | CEALCCPEPKLNNGEITQHRKSRPANHC |
| | | | | VYFYGDEISFSCHETSRFSAICQGDGTW |
| | | | | SPRTPSCGDICNFPPKIAHGHYKQSSSYS |
| | | | | FFKEEIIYECDKGYILVGQAKLSCSYSH |
| | | | | WSAPAPQCKALCRKPELVNGRLSVDK |
| | | | | DQYVEPENVTIQCDSGYGVVGPQSITCS |
| | | | | GNRTWYPEVPKCEWETPEGCEQVLTG |
| | | | | KRLMQCLPNPEDVKMALEVYKLSLEIE |
| | | | | QLELQRDSARQSTLDKEL |
| 463 | AGP2 | Alpha-1-acid | P19652 | MALSWVLTVLSLLPLLEAQIPLCANLVP |
| | | glycoprotein 2 | | VPITNATLDRITGKWFYIASAFRNEEYN |
| | | | | KSVQEIQATFFYFTPNKTEDTIFLREYQT |
| | | | | RQNQCFYNSSYLNVQRENGTVSRYEGG |
| | | | | REHVAHLLFLRDTKTLMFGSYLDDEKN |
| | | | | WGLSFYADKPETTKEQLGEFYEALDCL |
| | | | | CIPRSDVMYTDWKKDKCEPLEKQHEKE |
| | | | | RKQEEGES |
| 464 | KNG1 | Kininogen-1 | P01042 | MKLITILFLCSRLLLSLTQESQSEEIDCN |
| | | | | DKDLFKAVDAALKKYNSQNQSNNQFV |
| | | | | LYRITEATKTVGSDTFYSFKYEIKEGDC |
| | | | | PVQSGKTWQDCEYKDAAKAATGECTA |
| | | | | TVGKRSSTKFSVATQTCQITPAEGPVVT |
| | | | | AQYDCLGCVHPISTQSPDLEPILRHGIQ |
| | | | | YFNNNTQHSSLFMLNEVKRAQRQVVA |
| | | | | GLNFRITYSIVQTNCSKENFLFLTPDCKS |
| | | | | LWNGDTGECTDNAYIDIQLRIASFSQNC |
| | | | | DIYPGKDFVQPPTKICVGCPRDIPTNSPE |
| | | | | LEETLTHTITKLNAENNATFYFKIDNVK |
| | | | | KARVQVVAGKKYFIDFVARETTCSKES |
| | | | | NEELTESCETKKLGQSLDCNAEVYVVP |
| | | | | WEKKIYPTVNCQPLGMISLMKRPPGFSP |
| | | | | FRSSRIGEIKEETTVSPPHTSMAPAQDEE |
| | | | | RDSGKEQGHTRRHDWGHEKQRKHNLG |
| | | | | HGHKHERDQGHGHQRGHGLGHGHEQ |
| | | | | QHGLGHGHKFKLDDDLEHQGGHVLDH |
| | | | | GHKHKHGHGHGKHKNKGKKNGKHNG |
| | | | | WKTEHLASSSEDSTTPSAQTQEKTEGPT |
| | | | | PIPSLAKPGVTVTFSDFQDSDLIATMMP |
| | | | | PISPAPIQSDDDWIPDIQIDPNGLSFNPIS |
| | | | | DFPDTTSPKCPGRPWKSVSEINPTTQMK |
| | | | | ESYYFDLTDGLS |

Table 47 identifies and defines the glycan symbol structures included in Tables 41, 42, 43A, 43B, 43C, and 43D. Table 47 identifies a coded representation of the composition for each glycan structure included in Tables 41, 42, 43A, 43B, 43C, and 43D. As used herein, the 4-digit GL NO. is a designation that represents the number of hexoses, the number of HexNAcs, the number of Fucoses, and the number of Neuraminic Acids. It should be noted that glycan structure GL No 1102 is an O-glycan and the remaining glycans of Table 47 were N-glycans.

TABLE 47
Glycan Structure GL NOS: Composition

| Glycan | | | Glycan |
|---|---|---|---|
| Structure | Symbol | | Molecular |
| GL NO. | Structure | Composition | Weight |
| 1102 | [symbol] | Hex(1)HexNAc(1)Fuc(0)NeuAc(2) | 947.323008 |
| 3400 | [symbol] | Hex(3)HexNAc(4)Fuc(0)NeuAc(0) | 1298.47593 |
| 3410 | [symbol] | Hex(3)HexNAc(4)Fuc(1)NeuAc(0) | 1444.53384 |
| 3510 | [symbol] | Hex(3)HexNAc(5)Fuc(1)NeuAc(0) | 1647.61321 |
| 4300 | [symbol] | Hex(4)HexNAc(3)Fuc(0)NeuAc(0) | 1257.44938 |
| 4510 | [symbol] | Hex(4)HexNAc(5)Fuc(1)NeuAc(0) | 1809.66603 |
| 4511 | [symbol] | Hex(4)HexNAc(5)Fuc(1)NeuAc(1) | 2100.76144 |
| 5200 | [symbol] | Hex(5)HexNAc(2)Fuc(0)NeuAc(0) | 1216.42284 |
| 5301 | [symbol] | Hex(5)HexNAc(3)Fuc(0)NeuAc(1) | 1710.59761 |
| 5400 | [symbol] | Hex(5)HexNAc(4)Fuc(0)NeuAc(0) | 1622.58157 |
| 5401 | [symbol] | Hex(5)HexNAc(4)Fuc(0)NeuAc(1) | 1913.67698 |
| 5402 | [symbol] | Hex(5)HexNAc(4)Fuc(0)NeuAc(2) | 2204.77239 |
| 5411 | [symbol] | Hex(5)HexNAc(4)Fuc(1)NeuAc(1) | 2059.73489 |
| 5412 | [symbol] | Hex(5)HexNAc(4)Fuc(1)NeuAc(2) | 2350.8303 |
| 5421 | [symbol] | Hex(5)HexNAc(4)Fuc(2)NeuAc(1) | 2205.79279 |
| 5510 | [symbol] | Hex(5)HexNAc(5)Fuc(1)NeuAc(0) | 1971.71885 |
| 6501 | [symbol] | Hex(6)HexNAc(5)Fuc(0)NeuAc(1) | 2278.80917 |
| 6502 | [symbol] | Hex(6)HexNAc(5)Fuc(0)NeuAc(2) | 2569.90458 |
| 6503 | [symbol] | Hex(6)HexNAc(5)Fuc(0)NeuAc(3) | 2860.99999 |
| 6511 | [symbol] | Hex(6)HexNAc(5)Fuc(1)NeuAc(1) | 2424.86708 |
| 6512 | [symbol] | Hex(6)HexNAc(5)Fuc(1)NeuAc(2) | 2715.962 |
| 6513 | [symbol] | Hex(6)HexNAc(5)Fuc(1)NeuAc(3) | 3007.057896 |
| 6521 | [symbol] | Hex(6)HexNAc(5)Fuc(2)NeuAc(1) | |
| 7602 | [symbol] | Hex(7)HexNAc(6)Fuc(0)NeuAc(2) | 2935.03677 |
| 7603 | [symbol] | Hex(7)HexNAc(6)Fuc(0)NeuAc(3) | 3226.13218 |
| 7604 | [symbol] | Hex(7)HexNAc(6)Fuc(0)NeuAc(4) | 3517.227588 |
| 7612 | [symbol] | Hex(7)HexNAc(6)Fuc(1)Neu(5)Ac(2) | 3081.09467 |
| 7613 | [symbol] | Hex(7)HexNAc(6)Fuc(1)Neu(5)Ac(3) | 3372.190084 |
| 7614 | [symbol] | Hex(7)HexNAc(6)Fuc(1)NeuAc(4) | 3663.285494 |
| 8704 | [symbol] | Hex(8)HexNAc(7)Fuc(0)NeuAc(4) | 3882.35978 |
| 9804 | [symbol] | Hex(9)HexNAc(8)Fuc(0)NeuAc(4) | 4247.49196 |
| 11915 | [symbol] | Hex(11)HexNAc(9)Fuc(1)NeuAc(5) | 5211.830288 |
| 121005 | [symbol] | Hex(12)HexNAc(10)Fuc(0)NeuAc(5) | 5430.90457 |

Legend for Table 47

| | | | | |
|---|---|---|---|---|
| ● | [symbol] | [symbol] | [symbol] | [symbol] |
| Glc | Gal | Man | Fuc | Neu5Ac |
| ▪ | [symbol] | [symbol] | | |
| GlcNAc | GalNAc | ManNAc | | |

Table 47 illustrates the symbol structure and composition of detected glycan moieties that correspond to glycopeptides of Tables 41, 42, 43A, 43B, 43C, and 43D based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. It should be noted that the Glycan Structure GL NO 1102 is an O-linked glycan and that the rest of the glycans in Table 47 are N-linked glycans. For reference, N-linked glycans have a glycan attached to the amino acid asparagine and O-linked glycans have a glycan attached to either a serine or a threonine.

The identity of the various monosaccharides is illustrated by the Legend section located at the end of Table 47. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these clasess are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

Referring back to Table 47, for some entries, there are two symbol structures provided for one Glycan Structure GL NO such as, for example, Glycan Structure GL NO 3510. Thus, the identify of a peptide that references a Glycan Structure GL NO that has two symbol structures could be either one of the two possibilities based on the MRM of the LC-MS analysis. In some instances, a bracket symbol is used as part of the Symbol Structure to indicate that the precise bonding linkage is not exactly known, but that the linking line segment is attached to one of the plurality of adjacent carbohydrates immediately adjacent to the bracket. For example, the fucose of Glycan Structure GL NO 3510 could have either a core fucose or an outer-arm fucose linkage.

It should be noted that glycan symbol structure can illustrate an antennary format in the form of branches. For example, Glycan Structure GL NO's 6513 and 7604 show a tri-antennary and tetra-antennary sialic acid format, respectively.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an ovarian cancer disease state. A transition includes a precursor ion and at least one product ion grouping. As reviewed herein, the peptide structures in Tables 41, 42, 43A, 43B, 43C, and 43D as well as their corresponding precursor ion and product ion groupings in Tables 44A, 44B, and 44C (these ions having defined m/z ratios or m/z ratios that fall within the m/z ranges identified herein), can be used in mass spectrometry-based analyses to diagnose and facilitate treatment of diseases, such as, for example, PC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Tables 44A, 44B, and 44C or an m/z ratio within an identified m/z ratio as provided in Tables 44A, 44B, and 44C. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VIII. C. Peptide Structure and Product Ion Compositions and Kits—Melanoma

Aspects of the disclosure include compositions comprising one or more of the peptide structures listed in Table 55. In some embodiments, a composition comprises a plurality of the peptide structures listed in Table 55. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 of the peptide structures listed in Table 55. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOS: 570-595, listed in Table 55 and defined in Table 61 below.

Aspects of the disclosure include compositions comprising one or more precursor ions having a defined charge and/or defined mass-to-charge (m/z) ratio, as listed in Table 60A. Aspects of the disclosure include compositions comprising one or more product ions having a defined mass-to-charge (m/z) ratio, which product ions are produced by converting a peptide structure described herein (e.g., a peptide structure listed in Table 55) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 55). In some embodiments, a composition comprises a set of the product ions listed in Table 55, having an m/z ratio selected from the list provided for each peptide structure in Table 55.

In some embodiments, a composition comprises at least one of peptide structures PS-330 to PS-367 identified in Table 55.

In some embodiments, a composition comprises a peptide structure or a product ion. The peptide structure or product ion comprises an amino acid sequence having at least 90% sequence identity to any one of SEQ NOs: 21-46, as identified in Table 61, corresponding to peptide structures PS-330 to PS-367 in Table 55.

In some embodiments, a composition comprises a peptide structure having a monoisotopic mass identified in Table 55 as corresponding to the peptide structure.

In some embodiments, the product ion is selected as one from a group consisting of product ions identified in Table 60A, including product ions falling within an identified m/z range of the m/z ratio identified in Table 60A and characterized as having a precursor ion having an m/z ratio within an identified m/z range of the m/z ratio identified in Table 60A. A first range for the product ion m/z ratio may be ±0.5. A second range for the product ion m/z ratio may be ±0.8. A third range for the product ion m/z ratio may be ±1.0. A first range for the precursor ion m/z ratio may be ±0.5; a second range for the precursor ion m/z ratio may be ±1.0; a third range for the precursor ion m/z ratio may be ±1.5. Thus, a composition may include a product ion having an m/z ratio that falls within at least one of the first range (±0.5), the second range (±0.8), or the third range (±1.0) of the product ion m/z ratio identified in Table 60A, and characterized as having a precursor ion having an m/z ratio that falls within at least one of a first range (±0.5), a second range (±1.0), or a third range (±1.5) of the precursor ion m/z ratio identified in Table 60A.

TABLE 60A
Mass Spectrometry-Related Characteristics for the Peptide
Structures associated with Melanoma Treatments

| | | | | | 1st | 1st | 2nd | 2nd |
|---|---|---|---|---|---|---|---|---|
| PS-ID | RT | Collision | Precursor | Precursor | Product | Product | Product | Product |
| NO. | (min) | Energy | m/z | Charge | m/z | Charge | m/z | Charge |
| 330 | 7.8 | 22 | 938.4 | 3 | 366.1 | 1 | 1392.6 | 1 |
| 331 | 13.6 | 26 | 1073.1 | 3 | 366.1 | 1 | 1360.6 | 1 |
| 332 | 8 | 20 | 1054.7 | 3 | 366.1 | 1 | 1392.6 | 1 |
| 333 | 12.7 | 20 | 976.1 | 3 | 366.1 | 1 | 1360.6 | 1 |
| 334 | 7.8 | 24 | 987.1 | 3 | 366.1 | 1 | 1392.6 | 1 |
| 335 | 13.9 | 30 | 1019.4 | 3 | 204.1 | 1 | 1360.6 | 1 |
| 336 | 11.1 | 38 | 1295.2 | 3 | 366.1 | 1 | N/A | N/A |
| 337 | 12.8 | 25 | 1043.8 | 3 | 366.1 | 1 | 1360.6 | 1 |
| 338 | 39 | 27 | 1088.6 | 5 | 366.1 | 1 | N/A | N/A |
| 339 | 13.5 | 33 | 1073.4 | 4 | 366.1 | 1 | N/A | N/A |
| 340 | 41.5 | 26 | 1075.1 | 5 | 366.1 | 1 | 1056.2 | 3 |
| 341 | 30.4 | 20 | 1004.7 | 4 | 366.1 | 1 | N/A | N/A |
| 342 | 7.9 | 21 | 884.4 | 3 | 204.1 | 1 | 1392.6 | 1 |
| 343 | 30.2 | 28 | 1173.2 | 4 | 366.1 | 1 | 978.5 | 2 |
| 344 | 25 | 35 | 1152.4 | 3 | 366.1 | 1 | N/A | N/A |
| 345 | 27.3 | 24 | 995.4 | 4 | 366.1 | 1 | N/A | N/A |
| 346 | 40.8 | 20 | 1017.3 | 5 | 366.1 | 1 | 1584.4 | 2 |
| 347 | 13 | 23 | 941.1 | 3 | 204.1 | 1 | 1360.6 | 1 |
| 348 | 38 | 32 | 1287.7 | 4 | 366.1 | 1 | N/A | N/A |
| 349 | 23.3 | 33 | 991.4 | 4 | 366.1 | 1 | N/A | N/A |
| 350 | 34.4 | 23 | 1158.8 | 4 | 1206.9 | 3 | 366.1 | 1 |
| 351 | 14.8 | 25 | 984.7 | 4 | 366.1 | 1 | N/A | N/A |
| 352 | 12.1 | 35 | 1159.4 | 3 | 366.1 | 1 | N/A | N/A |
| 353 | 29.7 | 15 | 913.4 | 4 | 366.1 | 1 | 1234 | 2 |
| 354 | 9.9 | 15 | 927.4 | 3 | 204.1 | 1 | 1376.6 | 1 |
| 355 | 30.9 | 31 | 1004.7 | 4 | 366.1 | 1 | N/A | N/A |
| 356 | 26.2 | 28 | 1131.1 | 3 | 366.1 | 1 | 840.4 | 2 |
| 357 | 36.5 | 45 | 951.5 | 2 | 1178.5 | 1 | 1293.6 | 1 |
| 358 | 31.9 | 25 | 805.4 | 3 | 994.5 | 2 | 1044 | 2 |
| 359 | 34.4 | 30 | 1199.3 | 4 | 1206.9 | 3 | 366.1 | 1 |
| 360 | 34 | 30 | 1236.1 | 4 | 366.1 | 1 | N/A | N/A |
| 361 | 23.8 | 23 | 942.4 | 3 | 366.1 | 1 | 1114.6 | 1 |
| 362 | 31.1 | 28 | 1246 | 4 | 366.1 | 1 | 978.5 | 2 |
| 363 | 13 | 27 | 1116.4 | 5 | 366.1 | 1 | N/A | N/A |
| 364 | 29.1 | 31 | 1237.3 | 3 | 366.1 | 1 | 999.5 | 2 |
| 365 | 10.5 | 25 | 1024.5 | 3 | 204.1 | 1 | 1376.6 | 1 |
| 366 | 32.7 | 30 | 1182 | 4 | 366.1 | 1 | N/A | N/A |
| 367 | 33 | 20 | 1032.9 | 4 | 366.1 | 1 | 1208.6 | 2 |

TABLE 60B
Mass Spectrometry-Related Characteristics for the Peptide Structures
of Tables 58A.1 and 58A.2 associated with NSCLC Treatments

| Pept | Monoisotopic | | Collision | 1st | 1st | 2nd | 2nd | 1st | 2nd |
|---|---|---|---|---|---|---|---|---|---|
| SEQ | mass | RT | Energy | Precursor | Precursor | Precursor | Precursor | product | product |
| ID NO | (g/mol) | (min) | (V) | m/z | charge | m/z | charge | m/z | m/z |
| 613 | 5341.2196 | 37.3 | 30 | 1336.3 | 4 | N/A | N/A | 366.1 | N/A |
| 614 | 5375.373484 | 40.5 | 20 | 1076.5 | 5 | N/A | N/A | 274.1 | N/A |
| 615 | 959.503584 | 14.4 | 13 | 480.8 | 2 | N/A | N/A | 661.4 | 404.2 |
| 616 | 1825.968604 | 32.9 | 13 | 609.7 | 3 | N/A | N/A | 692.4 | 835.9 |
| 617 | 4218.871006 | 28.9 | 25 | 1055.7 | 4 | N/A | N/A | 366.1 | 999.5 |
| 618 | 2783.116514 | 23.8 | 30 | 928.7 | 3 | N/A | N/A | 366.1 | 1114.6 |
| 619 | 3104.4044 | 27.5 | 25 | 1036.1 | 3 | N/A | N/A | 274.1 | N/A |
| 620 | 5371.203674 | 30.4 | 34 | 1343.8 | 4 | N/A | N/A | 366.1 | N/A |
| 621 | 5385.40009 | 47.1 | 35 | 1347.9 | 4 | 1078.5 | 5 | 366.1 | 274.1 |
| 622 | 1245.62746 | 19.7 | 18 | 623.8 | 2 | N/A | N/A | 747.4 | 505.2 |
| 623 | 3302.3189 | 6.3 | 30 | 1101.8 | 3 | N/A | N/A | 366.1 | N/A |
| 624 | 4735.914404 | 29.8 | 25 | 1185.5 | 4 | N/A | N/A | 366.1 | 1368.1 |
| 625 | 3259.26547 | 10 | 30 | 1087.8 | 3 | N/A | N/A | 366.1 | 1112.5 |
| 626 | 2413.147086 | 31.9 | 25 | 805.4 | 3 | N/A | N/A | 994.5 | 1044 |
| 627 | 3180.627698 | 47.4 | 20 | 796.7 | 4 | N/A | N/A | 967.5 | 820.5 |
| 628 | 4883.15735 | 33.8 | 30 | 1222.2 | 4 | N/A | N/A | 366.1 | 1441.7 |
| 629 | 865.465746 | 13.6 | 11 | 433.7 | 2 | N/A | N/A | 696.4 | 533.3 |
| 630 | 3617.469324 | 16.8 | 20 | 905.6 | 4 | N/A | N/A | 366.1 | N/A |
| 631 | 2397.969344 | 13 | 40 | 1200.5 | 2 | N/A | N/A | 204.1 | N/A |
| 632 | 4163.729062 | 23.8 | 30 | 1042.4 | 4 | N/A | N/A | 366.1 | 848.1 |

TABLE 60C
Mass Spectrometry-Related Characteristics for the Peptide Structures
of Tables 58B.1 and 58B.2 associated with NSCLC Treatments

| Pept | Monoisotopic | | Collision | 1st | 1st | 2nd | 2nd | 1st | 2nd |
|---|---|---|---|---|---|---|---|---|---|
| SEQ | mass | RT | Energy | Precursor | Precursor | Precursor | Precursor | product | product |
| ID NO | (g/mol) | (min) | (V) | m/z | charge | m/z | charge | m/z | m/z |
| 653 | 5604.493466 | 42.3 | 30 | 1122.4 | 5 | N/A | N/A | 366.1 | 1299 |
| 654 | 5895.588876 | 42.9 | 29 | 1180.6 | 5 | N/A | N/A | 366.1 | 1299 |
| 655 | 6041.646782 | 42.8 | 30 | 1209.8 | 5 | N/A | N/A | 366.1 | 1299 |
| 656 | 5969.625654 | 42 | 30 | 1195.4 | 5 | N/A | N/A | 366.1 | 1299 |
| 657 | 6260.721064 | 42.8 | 30 | 1253.6 | 5 | N/A | N/A | 366.1 | 1299 |
| 658 | 6551.816474 | 43.3 | 33 | 1311.8 | 5 | N/A | N/A | 366.1 | 1299 |
| 659 | 6406.77897 | 42.7 | 30 | 1282.9 | 5 | N/A | N/A | 366.1 | 1299 |
| 660 | 6697.87438 | 43.3 | 34 | 1341 | 5 | N/A | N/A | 366.1 | 1299 |
| 661 | 3690.816484 | 42.5 | 25 | 924.3 | 4 | N/A | N/A | 833.9 | 782.3 |
| 662 | 3668.564882 | 37.5 | 30 | 1224.5 | 3 | N/A | N/A | 366.1 | 980 |
| 663 | 3959.660292 | 38.2 | 24 | 991.2 | 4 | N/A | N/A | 366.1 | 980.5 |
| 664 | 4105.718198 | 38 | 25 | 1027.7 | 4 | N/A | N/A | 366.1 | 980 |
| 665 | 4615.88789 | 38.3 | 30 | 1155.5 | 4 | 1540.3 | 3 | 274.1 | 366.1 |
| 666 | 1754.8879 | 39.2 | 30 | 879 | 2 | N/A | N/A | 545.3 | 952.5 |
| 667 | 5744.602348 | 42.4 | 28 | 1150.3 | 5 | 958.9 | 6 | 1249.3 | 366.1 |
| 668 | 5890.660254 | 42.4 | 29 | 1179.5 | 5 | N/A | N/A | 1248.6 | 366.1 |
| 669 | 5385.40009 | 47.1 | 35 | 1347.9 | 4 | 1078.5 | 5 | 366.1 | 274.1 |
| 670 | 5531.457996 | 47 | 27 | 1107.7 | 5 | 1384.4 | 4 | 366.1 | 366.1 |
| 671 | 3180.627698 | 47.4 | 20 | 796.7 | 4 | N/A | N/A | 967.5 | 820.5 |
| 672 | 6040.44195 | 33.3 | 15 | 1209.7 | 5 | N/A | N/A | 1347.9 | 366.1 |

Table 61 defines the peptide sequences for SEQ ID NOS: 570-595 from Table 1. Table 61 further identifies a corresponding protein SEQ TD NO for each peptide sequence. Each peptide sequence in Table 61 is defined as an amino acid sequence.

TABLE 61
Peptide SEQ ID NOS

| | | Corres- |
|---|---|---|
| SEQ | | ponding |
| ID | | Protein |
| NO: | Peptide Sequence | SEQ ID NO: |
| 570 | QIPLCANLVPVPITNATLDQITGK | 453 |
| 571 | EYESYSDFERNVTEK | 454 |
| 572 | LSLHRPALEDLLLGSEANLTCTLTGLR | 555, 594 |
| 573 | LQAPLNYTEFQKPICLPSK | 456 |
| 574 | YTGNASALFILPDQDK | 457 |
| 575 | WNCWSNWSSCSGR | 454 |
| 576 | VCQDCPLLAPLNDTR | 458 |
| 577 | LANLTQGEDQYYLR | 459 |
| 578 | SLGNVNFTVSAEALESQELCGTEVPSVPEH | 460 |
| | GR | |
| 579 | IPCSQPPQIEHGTINSSR | 461 |
| 580 | ISEENETTCYMGK | 461 |
| 581 | ALPQPQNVTSLLGCTH | 462 |
| 582 | EEQYNSTFR | 562, 593 |
| 583 | GVNFNVSK | 456 |
| 584 | CGLVPVLAENYNK | 464 |
| 585 | TTPPVLDSDGSFFLYSR | 493 |
| 586 | TPEVTCVVVDVSHEDPEVQFK | 463 |
| 587 | MVSHHNLTTGATLINEQWLLTTAK | 465 |
| 588 | NGSLFAFR | 466 |
| 589 | NLFLNHSENATAK | 465 |
| 590 | VVLHPNYSQVDIGLIK | 465 |
| 591 | LPTQNITFQTESSVAEQEAEFQSPK | 467 |
| 592 | TLNQSSDELQLSMGNAMFVK | 457 |
| 593 | VTACHSSQPNATLYK | 452 |
| 594 | EEQYNSTYR | 450 |
| 595 | EEQFNSTFR | 451 |

Table 62A identifies the proteins of SEQ ID NOS: 550-569 from Table 55. Table 62A identifies a corresponding protein abbreviation and protein name for each of protein SEQ ID NOS: 550-569. Further, Table 62A identifies a corresponding Uniprot TD for each of protein SEQ ID NOS: 550-569.

TABLE 62A
Protein SEQ ID NOS

| SEQ | Protein | | |
|---|---|---|---|
| ID | Abbre- | | Uniprot |
| NO. | viation | Protein Name | ID |
| 550 | IGG1 | Immunoglobulin heavy constant gamma 1 | P01857 |
| 551 | IGG2 | Immunoglobulin heavy constant gamma 2 | P01859 |
| 552 | THBG | Thyroxine-binding globulin | P05543 |
| 553 | AGP1 | Alpha-1-acid glycoprotein 1 | P02763 |
| 554 | CO8B | Complement component C8 beta chain | P07358 |
| 555 | IGA1 | Immunoglobulin heavy constant alpha 1 | P01876 |
| 556 | KLKB1 | Plasma kallikrein | P03952 |
| 557 | AACT | Alpha-1-antichymotrypsin | P01011 |
| 558 | FETUA | Alpha-2-HS-glycoprotein | P02765 |
| 559 | CLUS | Clusterin | P10909 |
| 560 | A2MG | Alpha-2-macroglobulin | P01023 |
| 561 | CFAH | Complement factor H | P08603 |
| 562 | HEMO | Hemopexin | P02790 |
| 563 | IGG3 | Immunoglobulin heavy constant gamma 3 | P01860 |
| 564 | TRFE | Serotransferrin | P02787 |
| 565 | HPT | Haptoglobin | P00738 |
| 566 | VTNC | Vitronectin | P04004 |
| 567 | ITIH4 | Inter-alpha-trypsin inhibitor heavy chain H4 | Q14624 |
| 568 | IGG4 | Immunoglobulin heavy constant gamma 4 | P01861 |
| 569 | IGA2 | Immunoglobulin heavy constant alpha 2 | P01877 |

Table 62B identifies the proteins of SEQ ID NOS: 593-612 from Table 58A.1 and Table 58A.2. Table 62B identifies a corresponding protein abbreviation, protein name, corresponding Uniprot ID for each of protein SEQ TD NOS: 593-612.

TABLE 62B
Protein SEQ ID NOS Corresponding to Table 58A.1 and Table 58A.2

| Protein | | | | |
|---|---|---|---|---|
| SEQ ID | Protein | | Uniprot | |
| NO. | Abbreviation | Protein Name | ID | Protein Sequence |
| 593 | VTNC | Vitronectin | P04004 | MAPLRPLLILALLAWVALADQESCKGRCTEGEN |
| | | | | VDKKCQCDELCSYYQSCCTDYTAECKPQVTRGD |
| | | | | VFTMPEDEYTVYDDGEEKNNATVHEQVGGPSLT |
| | | | | SDLQAQSKGNPEQTPVLKPEEEAPAPEVGASKPE |
| | | | | GIDSRPETLHPGRPQPPAEEELCSGKPFDAFTDLK |
| | | | | NGSLFAFRGQYCYELDEKAVRPGYPKLIRDVWG |
| | | | | IEGPIDAAFTRINCQGKTYLFKGSQYWRFEDGVL |
| | | | | DPDYPRNISDGFDGIPDNVDAALALPAHSYSGRE |
| | | | | RVYFFKGKQYWEYQFQHQPSQEECEGSSLSAVF |
| | | | | EHFAMMQRDSWEDIFELLFWGRTSAGTRQPQFIS |
| | | | | RDWHGVPGQVDAAMAGRIYISGMAPRPSLAKK |
| | | | | QRFRHRNRKGYRSQRGHSRGRNQNSRRPSRAT |
| | | | | WLSLFSSEESNLGANNYDDYRMDWLVPATCEPI |
| | | | | QSVFFFSGDKYYRVNLRTRRVDTVDPPYPRSIAQ |
| | | | | YWLGCPAPGHL |
| 594 | THRB | Prothrombin | P00734 | MAHVRGLQLPGCLALAALCSLVHSQHVFLAPQQ |
| | | | | ARSLLQRVRRANTFLEEVRKGNLERECVEETCSY |
| | | | | EEAFEALESSTATDVFWAKYTACETARTPRDKL |
| | | | | AACLEGNCAEGLGTNYRGHVNITRSGIECQLWR |
| | | | | SRYPHKPEINSTTHPGADLQENFCRNPDSSTTGP |
| | | | | WCYTTDPTVRRQECSIPVCGQDQVTVAMTPRSE |
| | | | | GSSVNLSPPLEQCVPDRGQQYQGRLAVTTHGLP |
| | | | | CLAWASAQAKALSKHQDENSAVQLVENFCRNP |
| | | | | DGDEEGVWCYVAGKPGDFGYCDLNYCEEAVEE |
| | | | | ETGDGLDEDSDRAIEGRTATSEYQTFFNPRTFGS |
| | | | | GEADCGLRPLFEKKSLEDKTERELLESYIDGRIVE |
| | | | | GSDAEIGMSPWQVMLFRKSPQELLCGASLISDR |
| | | | | WVLTAAHCLLYPPWDKNFTENDLLVRIGKHSRT |
| | | | | RYERNIEKISMLEKIYIHPRYNWRENLDRDIALM |
| | | | | KLKKPVAFSDYIHPVCLPDRETAASLLQAGYKG |
| | | | | RVTGWGNLKETWTANVGKGQPSVLQVVNLPIV |
| | | | | ERPVCKDSTRIRITDNMFCAGYKPDEGKRGDAC |
| | | | | EGDSGGPFVMKSPFNNRWYQMGIVSWGEGCDR |
| | | | | DGKYGFYTHVFRLKKWIQKVIDQFGE |
| 595 | AACT | Alpha-1- | P01011 | MERMLPLLALGLLAAGFCPAVLCHPNSPLDEEN |
| | | antichymotrypsin | | LTQENQDRGTHVDLGLASANVDFAFSLYKQLVL |
| | | | | KAPDKNVIFSPLSISTALAFLSLGAHNTTLTEILKG |
| | | | | LKFNLTETSEAEIHQSFQHLLRTLNQSSDELQLSM |
| | | | | GNAMFVKEQLSLLDRFTEDAKRLYGSEAFATDF |
| | | | | QDSAAAKKLINDYVKNGTRGKITDLIKDLDSQT |
| | | | | MMVLVNYIFFKAKWEMPFDPQDTHQSRFYLSK |
| | | | | KKWVMVPMMSLHHLTIPYFRDEELSCTVVELKY |
| | | | | TGNASALFILPDQDKMEEVEAMLLPETLKRWRD |
| | | | | SLEFREIGELYLPKFSISRDYNLNDILLQLGIEEAF |
| | | | | TSKADLSGITGARNLAVSQVVHKAVLDVFEEGT |
| | | | | EASAATAVKITLLSALVETRTIVRFNRPFLMIIVPT |
| | | | | DTQNIFFMSKVTNPKQA |
| 596 | IC1 | Plasmaprotease- | P05155 | MASRLTLLTLLLLLLAGDRASSNPNATSSSSQDP |
| | | C1inhibitor | | ESLQDRGEGKVATTVISKMLFVEPILEVSSLPTTN |
| | | | | STTNSATKITANTTDEPTTQPTTEPTTQPTIQPTQP |
| | | | | TTQLPTDSPTQPTTGSFCPGPVTLCSDLESHSTEA |
| | | | | VLGDALVDFSLKLYHAFSAMKKVETNMAFSPFS |
| | | | | IASLLTQVLLGAGENTKTNLESILSYPKDFTCVHQ |
| | | | | ALKGFTTKGVTSVSQIFHSPDLAIRDTFVNASRTL |
| | | | | YSSSPRVLSNNSDANLELINTWVAKNTNNKISRL |
| | | | | LDSLPSDTRLVLLNAIYLSAKWKTTFDPKKTRME |
| | | | | PFHFKNSVIKVPMMNSKKYPVAHFIDQTLKAKV |
| | | | | GQLQLSHNLSLVILVPQNLKHRLEDMEQALSPSV |
| | | | | FKAIMEKLEMSKFQPTLLTLPRIKVTTSQDMLSI |
| | | | | MEKLEFFDFSYDLNLCGLTEDPDLQVSAMQHQT |
| | | | | VLELTETGVEAAAASAISVARTLLVFEVQQPFLF |
| | | | | VLWDQQHKFPVFMGRVYDPRA |
| 597 | HPT | Haptoglobin | P00738 | MSALGAVIALLLWGQLFAVDSGNDVTDIADDGC |
| | | | | PKPPEIAHGYVEHSVRYQCKNYYKLRTEGDGVY |
| | | | | TLNDKKQWINKAVGDKLPECEADDGCPKPPEIA |
| | | | | HGYVEHSVRYQCKNYYKLRTEGDGVYTLNNEK |
| | | | | QWINKAVGDKLPECEAVCGKPKNPANPVQRILG |
| | | | | GHLDAKGSFPWQAKMVSHHNLTTGATLINEQW |
| | | | | LLTTAKNLFLNHSENATAKDIAPTLTLYVGKKQL |
| | | | | VEIEKVVLHPNYSQVDIGLIKLKQKVSVNERVMP |
| | | | | ICLPSKDYAEVGRVGYVSGWGRNANFKFTDHLK |
| | | | | YVMLPVADQDQCIRHYEGSTVPEKKTPKSPVGV |
| | | | | QPILNEHTFCAGMSKYQEDTCYGDAGSAFAVHD |
| | | | | LEEDTWYATGILSFDKSCAVAEYGVYVKVTSIQ |
| | | | | DWVQKTIAEN |
| 598 | VTNC | Vitronectin | P04004 | MAPLRPLLILALLAWVALADQESCKGRCTEGEN |
| | | | | VDKKCQCDELCSYYQSCCTDYTAECKPQVTRGD |
| | | | | VFTMPEDEYTVYDDGEEKNNATVHEQVGGPSLT |
| | | | | SDLQAQSKGNPEQTPVLKPEEEAPAPEVGASKPE |
| | | | | GIDSRPETLHPGRPQPPAEEELCSGKPFDAFTDLK |
| | | | | NGSLFAFRGQYCYELDEKAVRPGYPKLIRDVWG |
| | | | | IEGPIDAAFTRINCQGKTYLFKGSQYWRFEDGVL |
| | | | | DPDYPRNISDGFDGIPDNVDAALALPAHSYSGRE |
| | | | | RVYFFKGKQYWEYQFQHQPSQEECEGSSLSAVF |
| | | | | EHFAMMQRDSWEDIFELLFWGRTSAGTRQPQFIS |
| | | | | RDWHGVPGQVDAAMAGRIYISGMAPRPSLAKK |
| | | | | QRFRHRNRKGYRSQRGHSRGRNQNSRRPSRAT |
| | | | | WLSLFSSEESNLGANNYDDYRMDWLVPATCEPI |
| | | | | QSVFFFSGDKYYRVNLRTRRVDTVDPPYPRSIAQ |
| | | | | YWLGCPAPGHL |
| 599 | ITIH1 | Inter-alpha- | P19827 | MDGAMGPRGLLLCMYLVSLLILQAMPALGSAT |
| | | trypsinin- | | GRSKSSEKRQAVDTAVDGVFIRSLKVNCKVTSRF |
| | | hibitorheavychain | | AHYVVTSQVVNTANEAREVAFDLEIPKTAFISDF |
| | | H1 | | AVTADGNAFIGDIKDKVTAWKQYRKAAISGENA |
| | | | | GLVRASGRTMEQFTIHLTVNPQSKVTFQLTYEEV |
| | | | | LKRNHMQYEIVIKVKPKQLVHHFEIDVDIFEPQGI |
| | | | | SKLDAQASFLPKELAAQTIKKSFSGKKGHVLFRP |
| | | | | TVSQQQSCPTCSTSLLNGHFKVTYDVSRDKICDL |
| | | | | LVANNHFAHFFAPQNLTNMNKNVVFVIDISGSM |
| | | | | RGQKVKQTKEALLKILGDMQPGDYFDLVLFGTR |
| | | | | VQSWKGSLVQASEANLQAAQDFVRGFSLDEAT |
| | | | | NLNGGLLRGIEILNQVQESLPELSNHASILIMLTD |
| | | | | GDPTEGVTDRSQILKNVRNAIRGRFPLYNLGFGH |
| | | | | NVDFNFLEVMSMENNGRAQRIYEDHDATQQLQ |
| | | | | GFYSQVAKPLLVDVDLQYPQDAVLALTQNHHK |
| | | | | QYYEGSEIVVAGRIADNKQSSFKADVQAHGEGQ |
| | | | | EFSITCLVDEEEMKKLLRERGHMLENHVERLWA |
| | | | | YLTIQELLAKRMKVDREERANLSSQALQMSLDY |
| | | | | GFVTPLTSMSIRGMADQDGLKPTIDKPSEDSPPLE |
| | | | | MLGPRRTFVLSALQPSPTHSSSNTQRLPDRVTGV |
| | | | | DTDPHFIIHVPQKEDTLCFNINEEPGVILSLVQDP |
| | | | | NTGFSVNGQLIGNKARSPGQHDGTYFGRLGIANP |
| | | | | ATDFQLEVTPQNITLNPGFGGPVFSWRDQAVLR |
| | | | | QDGVVVTINKKRNLVVSVDDGGTFEVVLHRVW |
| | | | | KGSSVHQDFLGFYVLDSHRMSARTHGLLGQFFH |
| | | | | PIGFEVSDIHPGSDPTKPDATMVVRNRRLTVTRG |
| | | | | LQKDYSKDPWHGAEVSCWFIHNNGAGLIDGAY |
| | | | | TDYIVPDIF |
| 600 | FETUA | Alpha-2-HS- | P02765 | MKSLVLLLCLAQLWGCHSAPHGPGLIYRQPNCD |
| | | glycoprotein | | DPETEEAALVAIDYINQNLPWGYKHTLNQIDEVK |
| | | | | VWPQQPSGELFEIEIDTLETTCHVLDPTPVARCSV |
| | | | | RQLKEHAVEGDCDFQLLKLDGKFSVVYAKCDSS |
| | | | | PDSAEDVRKVCQDCPLLAPLNDTRVVHAAKAAL |
| | | | | AAFNAQNNGSNFQLEEISRAQLVPLPPSTYVEFT |
| | | | | VSGTDCVAKEATEAAKCNLLAEKQYGFCKATLS |
| | | | | EKLGGAEVAVTCMVFQTQPVSSQPQPEGANEAV |
| | | | | PTPVVDPDAPPSPPLGAPGLPPAGSPPDSHVLLAA |
| | | | | PPGHQLHRAHYDLRHTFMGVVSLGSPSGEVSHP |
| | | | | RKTRTVVQPSVGAAAGPVVPPCPGRIRHFKV |
| 601 | A1AT | Alpha-1- | P01009 | MPSSVSWGILLLAGLCCLVPVSLAEDPQGDAAQ |
| | | antitrypsin | | KTDTSHHDQDHPTFNKITPNLAEFAFSLYRQLAH |
| | | | | QSNSTNIFFSPVSIATAFAMLSLGTKADTHDEILE |
| | | | | GLNFNLTEIPEAQIHEGFQELLRTLNQPDSQLQLT |
| | | | | TGNGLFLSEGLKLVDKFLEDVKKLYHSEAFTVN |
| | | | | FGDTEEAKKQINDYVEKGTQGKIVDLVKELDRD |
| | | | | TVFALVNYIFFKGKWERPFEVKDTEEEDFHVDQ |
| | | | | VTTVKVPMMKRLGMFNIQHCKKLSSWVLLMKY |
| | | | | LGNATAIFFLPDEGKLQHLENELTHDIITKFLENE |
| | | | | DRRSASLHLPKLSITGTYDLKSVLGQLGITKVFSN |
| | | | | GADLSGVTEEAPLKLSKAVHKAVLTIDEKGTEA |
| | | | | AGAMFLEAIPMSIPPEVKFNKPFVFLMIEQNTKSP |
| | | | | LFMGKVVNPTQK |
| 602 | CO6 | Complement- | P13671 | MARRSVLYFILLNALINKGQACFCDHYAWTQWT |
| | | componentC6 | | SCSKTCNSGTQSRHRQIVVDKYYQENFCEQICSK |
| | | | | QETRECNWQRCPINCLLGDFGPWSDCDPCIEKQS |
| | | | | KVRSVLRPSQFGGQPCTAPLVAFQPCIPSKLCKIE |
| | | | | EADCKNKFRCDSGRCIARKLECNGENDCGDNSD |
| | | | | ERDCGRTKAVCTRKYNPIPSVQLMGNGFHFLAG |
| | | | | EPRGEVLDNSFTGGICKTVKSSRTSNPYRVPANL |
| | | | | ENVGFEVQTAEDDLKTDFYKDLTSLGHNENQQG |
| | | | | SFSSQGGSSFSVPIFYSSKRSENINHNSAFKQAIQA |
| | | | | SHKKDSSFIRIHKVMKVLNFTTKAKDLHLSDVFL |
| | | | | KALNHLPLEYNSALYSRIFDDFGTHYFTSGSLGG |
| | | | | VYDLLYQFSSEELKNSGLTEEEAKHCVRIETKKR |
| | | | | VLFAKKTKVEHRCTTNKLSEKHEGSFIQGAEKSI |
| | | | | SLIRGGRSEYGAALAWEKGSSGLEEKTFSEWLES |
| | | | | VKENPAVIDFELAPIVDLVRNIPCAVTKRNNLRK |
| | | | | ALQEYAAKFDPCQCAPCPNNGRPTLSGTECLCV |
| | | | | CQSGTYGENCEKQSPDYKSNAVDGQWGCWSSW |
| | | | | STCDATYKRSRTRECNNPAPQRGGKRCEGEKRQ |
| | | | | EEDCTFSIMENNGQPCINDDEEMKEVDLPEIEAD |
| | | | | SGCPQPVPPENGFIRNEKQLYLVGEDVEISCLTGF |
| | | | | ETVGYQYFRCLPDGTWRQGDVECQRTECIKPVV |
| | | | | QEVLTITPFQRLYRIGESIELTCPKGFVVAGPSRY |
| | | | | TCQGNSWTPPISNSLTCEKDTLTKLKGHCQLGQK |
| | | | | QSGSECICMSPEEDCSHHSEDLCVFDTDSNDYFT |
| | | | | SPACKFLAEKCLNNQQLHFLHIGSCQDGRQLEW |
| | | | | GLERTRLSSNSTKKESCGYDTCYDWEKCSASTS |
| | | | | KCVCLLPPQCFKGGNQLYCVKMGSSTSEKTLNIC |
| | | | | EVGTIRCANRKMEILHPGKCLA |
| 603 | IGM | Immunoglobulin- | P01871 | GSASAPTLFPLVSCENSPSDTSSVAVGCLAQDFLP |
| | | heavyconstantmu | | DSITFSWKYKNNSDISSTRGFPSVLRGGKYAATS |
| | | | | QVLLPSKDVMQGTDEHVVCKVQHPNGNKEKNV |
| | | | | PLPVIAELPPKVSVFVPPRDGFFGNPRKSKLICQA |
| | | | | TGFSPRQIQVSWLREGKQVGSGVTTDQVQAEAK |
| | | | | ESGPTTYKVTSTLTIKESDWLGQSMFTCRVDHRG |
| | | | | LTFQQNASSMCVPDQDTAIRVFAIPPSFASIFLTK |
| | | | | STKLTCLVTDLTTYDSVTISWTRQNGEAVKTHT |
| | | | | NISESHPNATFSAVGEASICEDDWNSGERFTCTV |
| | | | | THTDLPSPLKQTISRPKGVALHRPDVYLLPPARE |
| | | | | QLNLRESATITCLVTGFSPADVFVQWMQRGQPL |
| | | | | SPEKYVTSAPMPEPQAPGRYFAHSILTVSEEEWN |
| | | | | TGETYTCVVAHEALPNRVTERTVDKSTGKPTLY |
| | | | | NVSLVMSDTAGTCY |
| 604 | APOM | ApolipoproteinM | O95445 | MFHQIWAALLYFYGIILNSIYQCPEHSQLTTLGV |
| | | | | DGKEFPEVHLGQWYFIAGAAPTKEELATFDPVD |
| | | | | NIVFNMAAGSAPMQLHLRATIRMKDGLCVPRK |
| | | | | WIYHLTEGSTDLRTEGRPDMKTELFSSSCPGGIM |
| | | | | LNETGQGYQRFLLYNRSPHPPEKCVEEFKSLTSC |
| | | | | LDSKAFLLTPRNQEACELSNN |
| 605 | IC1 | Plasmaprotease- | P05155 | MASRLTLLTLLLLLLAGDRASSNPNATSSSSQDP |
| | | C1inhibitor | | ESLQDRGEGKVATTVISKMLFVEPILEVSSLPTTN |
| | | | | STTNSATKITANTTDEPTTQPTTEPTTQPTIQPTQP |
| | | | | TTQLPTDSPTQPTTGSFCPGPVTLCSDLESHSTEA |
| | | | | VLGDALVDFSLKLYHAFSAMKKVETNMAFSPFS |
| | | | | IASLLTQVLLGAGENTKTNLESILSYPKDFTCVHQ |
| | | | | ALKGFTTKGVTSVSQIFHSPDLAIRDTFVNASRTL |
| | | | | YSSSPRVLSNNSDANLELINTWVAKNTNNKISRL |
| | | | | LDSLPSDTRLVLLNAIYLSAKWKTTFDPKKTRME |
| | | | | PFHFKNSVIKVPMMNSKKYPVAHFIDQTLKAKV |
| | | | | GQLQLSHNLSLVILVPQNLKHRLEDMEQALSPSV |
| | | | | FKAIMEKLEMSKFQPTLLTLPRIKVTTSQDMLSI |
| | | | | MEKLEFFDFSYDLNLCGLTEDPDLQVSAMQHQT |
| | | | | VLELTETGVEAAAASAISVARTLLVFEVQQPFLF |
| | | | | VLWDQQHKFPVFMGRVYDPRA |
| 606 | IGG3 | Immunoglobulin- | P01860 | ASTKGPSVFPLAPCSRSTSGGTAALGCLVKDYFP |
| | | heavyconstant- | | EPVTVSWNSGALTSGVHTFPAVLQSSGLYSLSSV |
| | | gamma3 | | VTVPSSSLGTQTYTCNVNHKPSNTKVDKRVELK |
| | | | | TPLGDTTHTCPRCPEPKSCDTPPPCPRCPEPKSCD |
| | | | | TPPPCPRCPEPKSCDTPPPCPRCPAPELLGGPSVFL |
| | | | | FPPKPKDTLMISRTPEVTCVVVDVSHEDPEVQFK |
| | | | | WYVDGVEVHNAKTKPREEQYNSTFRVVSVLTV |
| | | | | LHQDWLNGKEYKCKVSNKALPAPIEKTISKTKG |
| | | | | QPREPQVYTLPPSREEMTKNQVSLTCLVKGFYPS |
| | | | | DIAVEWESSGQPENNYNTTPPMLDSDGSFFLYSK |
| | | | | LTVDKSRWQQGNIFSCSVMHEALHNRFTQKSLS |
| | | | | LSPGK |
| 607 | A1AT | Alpha-1- | P01009 | MPSSVSWGILLLAGLCCLVPVSLAEDPQGDAAQ |
| | | antitrypsin | | KTDTSHHDQDHPTFNKITPNLAEFAFSLYRQLAH |
| | | | | QSNSTNIFFSPVSIATAFAMLSLGTKADTHDEILE |
| | | | | GLNFNLTEIPEAQIHEGFQELLRTLNQPDSQLQLT |
| | | | | TGNGLFLSEGLKLVDKFLEDVKKLYHSEAFTVN |
| | | | | FGDTEEAKKQINDYVEKGTQGKIVDLVKELDRD |
| | | | | TVFALVNYIFFKGKWERPFEVKDTEEEDFHVDQ |
| | | | | VTTVKVPMMKRLGMFNIQHCKKLSSWVLLMKY |
| | | | | LGNATAIFFLPDEGKLQHLENELTHDIITKFLENE |
| | | | | DRRSASLHLPKLSITGTYDLKSVLGQLGITKVFSN |
| | | | | GADLSGVTEEAPLKLSKAVHKAVLTIDEKGTEA |
| | | | | AGAMFLEAIPMSIPPEVKFNKPFVFLMIEQNTKSP |
| | | | | LFMGKVVNPTQK |
| 608 | HPT | Haptoglobin | P00738 | MSALGAVIALLLWGQLFAVDSGNDVTDIADDGC |
| | | | | PKPPEIAHGYVEHSVRYQCKNYYKLRTEGDGVY |
| | | | | TLNDKKQWINKAVGDKLPECEADDGCPKPPEIA |
| | | | | HGYVEHSVRYQCKNYYKLRTEGDGVYTLNNEK |
| | | | | QWINKAVGDKLPECEAVCGKPKNPANPVQRILG |
| | | | | GHLDAKGSFPWQAKMVSHHNLTTGATLINEQW |
| | | | | LLTTAKNLFLNHSENATAKDIAPTLTLYVGKKQL |
| | | | | VEIEKVVLHPNYSQVDIGLIKLKQKVSVNERVMP |
| | | | | ICLPSKDYAEVGRVGYVSGWGRNANFKFTDHLK |
| | | | | YVMLPVADQDQCIRHYEGSTVPEKKTPKSPVGV |
| | | | | QPILNEHTFCAGMSKYQEDTCYGDAGSAFAVHD |
| | | | | LEEDTWYATGILSFDKSCAVAEYGVYVKVTSIQ |
| | | | | DWVQKTIAEN |
| 609 | CO5 | ComplementC5 | P01031 | MGLLGILCFLIFLGKTWGQEQTYVISAPKIFRVGASEN |
| | | | | IVIQVYGYTEAFDATISIKSYPDKKFSYSSGHVHLSSE |
| | | | | NKFQNSAILTIQPKQLPGGQNPVSYVYLEVVSKHFSKS |
| | | | | KRMPITYDNGFLFIHTDKPVYTPDQSVKVRVYSLNDDL |
| | | | | KPAKRETVLTFIDPEGSEVDMVEEIDHIGIISFPDFKI |
| | | | | PSNPRYGMWTIKAKYKEDFSTTGTAYFEVKEYVLPHFS |
| | | | | VSIEPEYNFIGYKNFKNFEITIKARYFYNKVVTEADVY |
| | | | | ITFGIREDLKDDQKEMMQTAMQNTMLINGIAQVTFDSE |
| | | | | TAVKELSYYSLEDLNNKYLYIAVTVIESTGGFSEEAEI |
| | | | | PGIKYVLSPYKLNLVATPLFLKPGIPYPIKVQVKDSLD |
| | | | | QLVGGVPVTLNAQTIDVNQETSDLDPSKSVTRVDDGVA |
| | | | | SFVLNLPSGVTVLEFNVKTDAPDLPEENQAREGYRAIA |
| | | | | YSSLSQSYLYIDWTDNHKALLVGEHLNIIVTPKSPYID |
| | | | | KITHYNYLILSKGKIIHFGTREKFSDASYQSINIPVTQ |
| | | | | NMVPSSRLLVYYIVTGEQTAELVSDSVWLNIEEKCGNQ |
| | | | | LQVHLSPDADAYSPGQTVSLNMATGMDSWVALAAVDSA |
| | | | | VYGVQRGAKKPLERVFQFLEKSDLGCGAGGGLNNANVF |
| | | | | HLAGLTFLTNANADDSQENDEPCKEILRPRRTLQKKIE |
| | | | | EIAAKYKHSVVKKCCYDGACVNNDETCEQRAARISLGP |
| | | | | RCIKAFTECCVVASQLRANISHKDMQLGRLHMKTLLPV |
| | | | | SKPEIRSYFPESWLWEVHLVPRRKQLQFALPDSLTTWE |
| | | | | IQGVGISNTGICVADTVKAKVFKDVFLEMNIPYSVVRG |
| | | | | EQIQLKGTVYNYRTSGMQFCVKMSAVEGICTSESPVID |
| | | | | HQGTKSSKCVRQKVEGSSSHLVTFTVLPLEIGLHNINF |
| | | | | SLETWFGKEILVKTLRVVPEGVKRESYSGVTLDPRGIY |
| | | | | GTISRRKEFPYRIPLDLVPKTEIKRILSVKGLLVGEIL |
| | | | | SAVLSQEGINILTHLPKGSAEAELMSVVPVFYVFHYLE |
| | | | | TGNHWNIFHSDPLIEKQKLKKKLKEGMLSIMSYRNADY |
| | | | | SYSVWKGGSASTWLTAFALRVLGQVNKYVEQNONSICN |
| | | | | SLLWLVENYQLDNGSFKENSQYQPIKLQGTLPVEAREN |
| | | | | SLYLTAFTVIGIRKAFDICPLVKIDTALIKADNFLLEN |
| | | | | TLPAQSTFTLAISAYALSLGDKTHPQFRSIVSALKREA |
| | | | | LVKGNPPIYRFWKDNLQHKDSSVPNTGTARMVETTAYA |
| | | | | LLTSLNLKDINYVNPVIKWLSEEQRYGGGFYSTQDTIN |
| | | | | AIEGLTEYSLLVKQLRLSMDIDVSYKHKGALHNYKMTD |
| | | | | KNFLGRPVEVLLNDDLIVSTGFGSGLATVHVTTVVHKT |
| | | | | STSEEVCSFYLKIDTQDIEASHYRGYGNSDYKRIVACA |
| | | | | SYKPSREESSSGSSHAVMDISLPTGISANEEDLKALVE |
| | | | | GVDQLFTDYQIKDGHVILQLNSIPSSDFLCVRFRIFEL |
| | | | | FEVGFLSPATFTVYEYHRPDKQCTMFYSTSNIKIQKVC |
| | | | | EGAACKCVEADCGQMQEELDLTISAETRKQTACKPEIA |
| | | | | YAYKVSITSITVENVFVKYKATLLDIYKTGEAVAEKDS |
| | | | | EITFIKKVTCTNAELVKGRQYLIMGKEALQIKYNFSFR |
| | | | | YIYPLDSLTWIEYWPRDTTCSSCQAFLANLDEFAEDIF |
| | | | | LNGC |
| 610 | KNG1 | Kininogen-1 | P01042 | MKLITILFLCSRLLLSLTQESQSEEIDCNDKDLFK |
| | | | | AVDAALKKYNSQNQSNNQFVLYRITEATKTVGS |
| | | | | DTFYSFKYEIKEGDCPVQSGKTWQDCEYKDAAK |
| | | | | AATGECTATVGKRSSTKFSVATQTCQITPAEGPV |
| | | | | VTAQYDCLGCVHPISTQSPDLEPILRHGIQYFNNN |
| | | | | TQHSSLFMLNEVKRAQRQVVAGLNFRITYSIVQT |
| | | | | NCSKENFLFLTPDCKSLWNGDTGECTDNAYIDIQ |
| | | | | LRIASFSQNCDIYPGKDFVQPPTKICVGCPRDIPT |
| | | | | NSPELEETLTHTITKLNAENNATFYFKIDNVKKA |
| | | | | RVQVVAGKKYFIDFVARETTCSKESNEELTESCE |
| | | | | TKKLGQSLDCNAEVYVVPWEKKIYPTVNCQPLG |
| | | | | MISLMKRPPGFSPFRSSRIGEIKEETTVSPPHTSMA |
| | | | | PAQDEERDSGKEQGHTRRHDWGHEKQRKHNLG |
| | | | | HGHKHERDQGHGHQRGHGLGHGHEQQHGLGH |
| | | | | GHKFKLDDDLEHQGGHVLDHGHKHKHGHGHG |
| | | | | KHKNKGKKNGKHNGWKTEHLASSSEDSTTPSA |
| | | | | QTQEKTEGPTPIPSLAKPGVTVTFSDFQDSDLIAT |
| | | | | MMPPISPAPIQSDDDWIPDIQIDPNGLSFNPISDFP |
| | | | | DTTSPKCPGRPWKSVSEINPTTQMKESYYFDLTD |
| | | | | GLS |
| 611 | IGG2 | Immunoglobulin- | P01859 | ASTKGPSVFPLAPCSRSTSESTAALGCLVKDYFPE |
| | | heavyconstant- | | PVTVSWNSGALTSGVHTFPAVLQSSGLYSLSSVV |
| | | gamma2 | | TVPSSNFGTQTYTCNVDHKPSNTKVDKTVERKC |
| | | | | CVECPPCPAPPVAGPSVFLFPPKPKDTLMISRTPE |
| | | | | VTCVVVDVSHEDPEVQFNWYVDGVEVHNAKTK |
| | | | | PREEQFNSTFRVVSVLTVVHQDWLNGKEYKCKV |
| | | | | SNKGLPAPIEKTISKTKGQPREPQVYTLPPSREEM |
| | | | | TKNQVSLTCLVKGFYPSDISVEWESNGQPENNY |
| | | | | KTTPPMLDSDGSFFLYSKLTVDKSRWQQGNVFS |
| | | | | CSVMHEALHNHYTQKSLSLSPGK |
| 612 | IGM | Immunoglobulin- | P01871 | GSASAPTLFPLVSCENSPSDTSSVAVGCLAQDFLP |
| | | heavyconstantmu | | DSITFSWKYKNNSDISSTRGFPSVLRGGKYAATS |
| | | | | QVLLPSKDVMQGTDEHVVCKVQHPNGNKEKNV |
| | | | | PLPVIAELPPKVSVFVPPRDGFFGNPRKSKLICQA |
| | | | | TGFSPRQIQVSWLREGKQVGSGVTTDQVQAEAK |
| | | | | ESGPTTYKVTSTLTIKESDWLGQSMFTCRVDHRG |
| | | | | LTFQQNASSMCVPDQDTAIRVFAIPPSFASIFLTK |
| | | | | STKLTCLVTDLTTYDSVTISWTRQNGEAVKTHT |
| | | | | NISESHPNATFSAVGEASICEDDWNSGERFTCTV |
| | | | | THTDLPSPLKQTISRPKGVALHRPDVYLLPPARE |
| | | | | QLNLRESATITCLVTGFSPADVFVQWMQRGQPL |
| | | | | SPEKYVTSAPMPEPQAPGRYFAHSILTVSEEEWN |
| | | | | TGETYTCVVAHEALPNRVTERTVDKSTGKPTLY |
| | | | | NVSLVMSDTAGTCY |

Table 62C identifies the proteins of SEQ ID NOS: 633-652 from Table 581B.1 and Table 58B.2. Table 62C identifies a corresponding protein abbreviation, protein name, corresponding Uniprot ID for each of protein SEQ TD NOS: 633-652.

TABLE 62C
Protein SEQ ID NOS Corresponding to Table 58B.1 and Table 58B.2

| Protein | | | | |
|---|---|---|---|---|
| SEQ ID | Protein | | Uniprot | |
| NO. | Abbreviation | Protein Name | ID | ProteinSequence |
| 633 | AACT | Alpha-1- | P01011 | MERMLPLLALGLLAAGFCPAVLCHPNSPLDEEN |
| | | antichymotrypsin | | LTQENQDRGTHVDLGLASANVDFAFSLYKQLVL |
| | | | | KAPDKNVIFSPLSISTALAFLSLGAHNTTLTEILKG |
| | | | | LKFNLTETSEAEIHQSFQHLLRTLNQSSDELQLSM |
| | | | | GNAMFVKEQLSLLDRFTEDAKRLYGSEAFATDF |
| | | | | QDSAAAKKLINDYVKNGTRGKITDLIKDLDSQT |
| | | | | MMVLVNYIFFKAKWEMPFDPQDTHQSRFYLSK |
| | | | | KKWVMVPMMSLHHLTIPYFRDEELSCTVVELKY |
| | | | | TGNASALFILPDQDKMEEVEAMLLPETLKRWRD |
| | | | | SLEFREIGELYLPKFSISRDYNLNDILLQLGIEEAF |
| | | | | TSKADLSGITGARNLAVSQVVHKAVLDVFEEGT |
| | | | | EASAATAVKITLLSALVETRTIVRFNRPFLMIIVPT |
| | | | | DTQNIFFMSKVTNPKQA |
| 634 | APOM | ApolipoproteinM | O95445 | MFHQIWAALLYFYGIILNSIYQCPEHSQLTTLGV |
| | | | | DGKEFPEVHLGQWYFIAGAAPTKEELATFDPVD |
| | | | | NIVFNMAAGSAPMQLHLRATIRMKDGLCVPRK |
| | | | | WIYHLTEGSTDLRTEGRPDMKTELFSSSCPGGIM |
| | | | | LNETGQGYQRFLLYNRSPHPPEKCVEEFKSLTSC |
| | | | | LDSKAFLLTPRNQEACELSNN |
| 635 | AGP1 | Alpha-1-acid | P02763 | MALSWVLTVLSLLPLLEAQIPLCANLVPVPITNA |
| | | glycoprotein 1 | | TLDRITGKWFYIASAFRNEEYNKSVQEIQATFFYF |
| | | | | TPNKTEDTIFLREYQTRQDQCIYNTTYLNVQREN |
| | | | | GTISRYVGGQEHFAHLLILRDTKTYMLAFDVND |
| | | | | EKNWGLSVYADKPETTKEQLGEFYEALDCLRIP |
| | | | | KSDVVYTDWKKDKCEPLEKQHEKERKQEEGES |
| 636 | IC1 | Plasma protease | P05155 | MASRLTLLTLLLLLLAGDRASSNPNATSSSSQDP |
| | | C1 inhibitor | | ESLQDRGEGKVATTVISKMLFVEPILEVSSLPTTN |
| | | | | STTNSATKITANTTDEPTTQPTTEPTTQPTIQPTQP |
| | | | | TTQLPTDSPTQPTTGSFCPGPVTLCSDLESHSTEA |
| | | | | VLGDALVDFSLKLYHAFSAMKKVETNMAFSPFS |
| | | | | IASLLTQVLLGAGENTKTNLESILSYPKDFTCVHQ |
| | | | | ALKGFTTKGVTSVSQIFHSPDLAIRDTFVNASRTL |
| | | | | YSSSPRVLSNNSDANLELINTWVAKNTNNKISRL |
| | | | | LDSLPSDTRLVLLNAIYLSAKWKTTFDPKKTRME |
| | | | | PFHFKNSVIKVPMMNSKKYPVAHFIDQTLKAKV |
| | | | | GQLQLSHNLSLVILVPQNLKHRLEDMEQALSPSV |
| | | | | FKAIMEKLEMSKFQPTLLTLPRIKVTTSQDMLSI |
| | | | | MEKLEFFDFSYDLNLCGLTEDPDLQVSAMQHQT |
| | | | | VLELTETGVEAAAASAISVARTLLVFEVQQPFLF |
| | | | | VLWDQQHKFPVFMGRVYDPRA |
| 637 | AGP2 | Alpha-1-acid | P19652 | MALSWVLTVLSLLPLLEAQIPLCANLVPVPITNA |
| | | glycoprotein | | TLDRITGKWFYIASAFRNEEYNKSVQEIQATFFYF |
| | | 1or2 | | TPNKTEDTIFLREYQTRQNQCFYNSSYLNVQREN |
| | | | | GTVSRYEGGREHVAHLLFLRDTKTLMFGSYLDD |
| | | | | EKNWGLSFYADKPETTKEQLGEFYEALDCLCIPR |
| | | | | SDVMYTDWKKDKCEPLEKQHEKERKQEEGES |
| 638 | CO5 | ComplementC5 | P01031 | MGLLGILCFLIFLGKTWGQEQTYVISAPKIFRVGA |
| | | | | SENIVIQVYGYTEAFDATISIKSYPDKKFSYSSGH |
| | | | | VHLSSENKFQNSAILTIQPKQLPGGQNPVSYVYL |
| | | | | EVVSKHFSKSKRMPITYDNGFLFIHTDKPVYTPD |
| | | | | QSVKVRVYSLNDDLKPAKRETVLTFIDPEGSEVD |
| | | | | MVEEIDHIGIISFPDFKIPSNPRYGMWTIKAKYKE |
| | | | | DFSTTGTAYFEVKEYVLPHFSVSIEPEYNFIGYKN |
| | | | | FKNFEITIKARYFYNKVVTEADVYITFGIREDLKD |
| | | | | DQKEMMQTAMQNTMLINGIAQVTFDSETAVKE |
| | | | | LSYYSLEDLNNKYLYIAVTVIESTGGFSEEAEIPGI |
| | | | | KYVLSPYKLNLVATPLFLKPGIPYPIKVQVKDSL |
| | | | | DQLVGGVPVTLNAQTIDVNQETSDLDPSKSVTR |
| | | | | VDDGVASFVLNLPSGVTVLEFNVKTDAPDLPEE |
| | | | | NQAREGYRAIAYSSLSQSYLYIDWTDNHKALLV |
| | | | | GEHLNIIVTPKSPYIDKITHYNYLILSKGKIIHFGT |
| | | | | REKFSDASYQSINIPVTQNMVPSSRLLVYYIVTGE |
| | | | | QTAELVSDSVWLNIEEKCGNQLQVHLSPDADAY |
| | | | | SPGQTVSLNMATGMDSWVALAAVDSAVYGVQ |
| | | | | RGAKKPLERVFQFLEKSDLGCGAGGGLNNANVF |
| | | | | HLAGLTFLTNANADDSQENDEPCKEILRPRRTLQ |
| | | | | KKIEEIAAKYKHSVVKKCCYDGACVNNDETCEQ |
| | | | | RAARISLGPRCIKAFTECCVVASQLRANISHKDM |
| | | | | QLGRLHMKTLLPVSKPEIRSYFPESWLWEVHLVP |
| | | | | RRKQLQFALPDSLTTWEIQGVGISNTGICVADTV |
| | | | | KAKVFKDVFLEMNIPYSVVRGEQIQLKGTVYNY |
| | | | | RTSGMQFCVKMSAVEGICTSESPVIDHQGTKSSK |
| | | | | CVRQKVEGSSSHLVTFTVLPLEIGLHNINFSLETW |
| | | | | FGKEILVKTLRVVPEGVKRESYSGVTLDPRGIYG |
| | | | | TISRRKEFPYRIPLDLVPKTEIKRILSVKGLLVGEI |
| | | | | LSAVLSQEGINILTHLPKGSAEAELMSVVPVFYV |
| | | | | FHYLETGNHWNIFHSDPLIEKQKLKKKLKEGML |
| | | | | SIMSYRNADYSYSVWKGGSASTWLTAFALRVLG |
| | | | | QVNKYVEQNQNSICNSLLWLVENYQLDNGSFKE |
| | | | | NSQYQPIKLQGTLPVEARENSLYLTAFTVIGIRKA |
| | | | | FDICPLVKIDTALIKADNFLLENTLPAQSTFTLAIS |
| | | | | AYALSLGDKTHPQFRSIVSALKREALVKGNPPIY |
| | | | | RFWKDNLQHKDSSVPNTGTARMVETTAYALLTS |
| | | | | LNLKDINYVNPVIKWLSEEQRYGGGFYSTQDTIN |
| | | | | AIEGLTEYSLLVKQLRLSMDIDVSYKHKGALHN |
| | | | | YKMTDKNFLGRPVEVLLNDDLIVSTGFGSGLAT |
| | | | | VHVTTVVHKTSTSEEVCSFYLKIDTQDIEASHYR |
| | | | | GYGNSDYKRIVACASYKPSREESSSGSSHAVMDI |
| | | | | SLPTGISANEEDLKALVEGVDQLFTDYQIKDGHV |
| | | | | ILQLNSIPSSDFLCVRFRIFELFEVGFLSPATFTVYE |
| | | | | YHRPDKQCTMFYSTSNIKIQKVCEGAACKCVEA |
| | | | | DCGQMQEELDLTISAETRKQTACKPEIAYAYKVS |
| | | | | ITSITVENVFVKYKATLLDIYKTGEAVAEKDSEIT |
| | | | | FIKKVTCTNAELVKGRQYLIMGKEALQIKYNFSF |
| | | | | RYIYPLDSLTWIEYWPRDTTCSSCQAFLANLDEF |
| | | | | AEDIFLNGC |
| 639 | FETUA | Alpha-2-HS- | P02765 | MKSLVLLLCLAQLWGCHSAPHGPGLIYRQPNCD |
| | | glycoprotein | | DPETEEAALVAIDYINQNLPWGYKHTLNQIDEVK |
| | | | | VWPQQPSGELFEIEIDTLETTCHVLDPTPVARCSV |
| | | | | RQLKEHAVEGDCDFQLLKLDGKFSVVYAKCDSS |
| | | | | PDSAEDVRKVCQDCPLLAPLNDTRVVHAAKAAL |
| | | | | AAFNAQNNGSNFQLEEISRAQLVPLPPSTYVEFT |
| | | | | VSGTDCVAKEATEAAKCNLLAEKQYGFCKATLS |
| | | | | EKLGGAEVAVTCMVFQTQPVSSQPQPEGANEAV |
| | | | | PTPVVDPDAPPSPPLGAPGLPPAGSPPDSHVLLAA |
| | | | | PPGHQLHRAHYDLRHTFMGVVSLGSPSGEVSHP |
| | | | | RKTRTVVQPSVGAAAGPVVPPCPGRIRHFKV |
| 640 | FETUA | Alpha-2-HS- | P02765 | MKSLVLLLCLAQLWGCHSAPHGPGLIYRQPNCD |
| | | glycoprotein | | DPETEEAALVAIDYINQNLPWGYKHTLNQIDEVK |
| | | | | VWPQQPSGELFEIEIDTLETTCHVLDPTPVARCSV |
| | | | | RQLKEHAVEGDCDFQLLKLDGKFSVVYAKCDSS |
| | | | | PDSAEDVRKVCQDCPLLAPLNDTRVVHAAKAAL |
| | | | | AAFNAQNNGSNFQLEEISRAQLVPLPPSTYVEFT |
| | | | | VSGTDCVAKEATEAAKCNLLAEKQYGFCKATLS |
| | | | | EKLGGAEVAVTCMVFQTQPVSSQPQPEGANEAV |
| | | | | PTPVVDPDAPPSPPLGAPGLPPAGSPPDSHVLLAA |
| | | | | PPGHQLHRAHYDLRHTFMGVVSLGSPSGEVSHP |
| | | | | RKTRTVVQPSVGAAAGPVVPPCPGRIRHFKV |
| 641 | CO6 | Complement- | P13671 | MARRSVLYFILLNALINKGQACFCDHYAWTQWT |
| | | componentC6 | | SCSKTCNSGTQSRHRQIVVDKYYQENFCEQICSK |
| | | | | QETRECNWQRCPINCLLGDFGPWSDCDPCIEKQS |
| | | | | KVRSVLRPSQFGGQPCTAPLVAFQPCIPSKLCKIE |
| | | | | EADCKNKFRCDSGRCIARKLECNGENDCGDNSD |
| | | | | ERDCGRTKAVCTRKYNPIPSVQLMGNGFHFLAG |
| | | | | EPRGEVLDNSFTGGICKTVKSSRTSNPYRVPANL |
| | | | | ENVGFEVQTAEDDLKTDFYKDLTSLGHNENQQG |
| | | | | SFSSQGGSSFSVPIFYSSKRSENINHNSAFKQAIQA |
| | | | | SHKKDSSFIRIHKVMKVLNFTTKAKDLHLSDVFL |
| | | | | KALNHLPLEYNSALYSRIFDDFGTHYFTSGSLGG |
| | | | | VYDLLYQFSSEELKNSGLTEEEAKHCVRIETKKR |
| | | | | VLFAKKTKVEHRCTTNKLSEKHEGSFIQGAEKSI |
| | | | | SLIRGGRSEYGAALAWEKGSSGLEEKTFSEWLES |
| | | | | VKENPAVIDFELAPIVDLVRNIPCAVTKRNNLRK |
| | | | | ALQEYAAKFDPCQCAPCPNNGRPTLSGTECLCV |
| | | | | CQSGTYGENCEKQSPDYKSNAVDGQWGCWSSW |
| | | | | STCDATYKRSRTRECNNPAPQRGGKRCEGEKRQ |
| | | | | EEDCTFSIMENNGQPCINDDEEMKEVDLPEIEAD |
| | | | | SGCPQPVPPENGFIRNEKQLYLVGEDVEISCLTGF |
| | | | | ETVGYQYFRCLPDGTWRQGDVECQRTECIKPVV |
| | | | | QEVLTITPFQRLYRIGESIELTCPKGFVVAGPSRY |
| | | | | TCQGNSWTPPISNSLTCEKDTLTKLKGHCQLGQK |
| | | | | QSGSECICMSPEEDCSHHSEDLCVFDTDSNDYFT |
| | | | | SPACKFLAEKCLNNQQLHFLHIGSCQDGRQLEW |
| | | | | GLERTRLSSNSTKKESCGYDTCYDWEKCSASTS |
| | | | | KCVCLLPPQCFKGGNQLYCVKMGSSTSEKTLNIC |
| | | | | EVGTIRCANRKMEILHPGKCLA |
| 642 | HPT | Haptoglobin | P00738 | MSALGAVIALLLWGQLFAVDSGNDVTDIADDGC |
| | | | | PKPPEIAHGYVEHSVRYQCKNYYKLRTEGDGVY |
| | | | | TLNDKKQWINKAVGDKLPECEADDGCPKPPEIA |
| | | | | HGYVEHSVRYQCKNYYKLRTEGDGVYTLNNEK |
| | | | | QWINKAVGDKLPECEAVCGKPKNPANPVQRILG |
| | | | | GHLDAKGSFPWQAKMVSHHNLTTGATLINEQW |
| | | | | LLTTAKNLFLNHSENATAKDIAPTLTLYVGKKQL |
| | | | | VEIEKVVLHPNYSQVDIGLIKLKQKVSVNERVMP |
| | | | | ICLPSKDYAEVGRVGYVSGWGRNANFKFTDHLK |
| | | | | YVMLPVADQDQCIRHYEGSTVPEKKTPKSPVGV |
| | | | | QPILNEHTFCAGMSKYQEDTCYGDAGSAFAVHD |
| | | | | LEEDTWYATGILSFDKSCAVAEYGVYVKVTSIQ |
| | | | | DWVQKTIAEN |
| 643 | APOB | Apolipoprotein | P04114 | MDPPRPALLALLALPALLLLLLAGARAEEEMLE |
| | | B-100 | | NVSLVCPKDATRFKHLRKYTYNYEAESSSGVPG |
| | | | | TADSRSATRINCKVELEVPQLCSFILKTSQCTLKE |
| | | | | VYGFNPEGKALLKKTKNSEEFAAAMSRYELKLA |
| | | | | IPEGKQVFLYPEKDEPTYILNIKRGIISALLVPPET |
| | | | | EEAKQVLFLDTVYGNCSTHFTVKTRKGNVATEIS |
| | | | | TERDLGQCDRFKPIRTGISPLALIKGMTRPLSTLIS |
| | | | | SSQSCQYTLDAKRKHVAEAICKEQHLFLPFSYKN |
| | | | | KYGMVAQVTQTLKLEDTPKINSRFFGEGTKKMG |
| | | | | LAFESTKSTSPPKQAEAVLKTLQELKKLTISEQNI |
| | | | | QRANLFNKLVTELRGLSDEAVTSLLPQLIEVSSPI |
| | | | | TLQALVQCGQPQCSTHILQWLKRVHANPLLIDV |
| | | | | VTYLVALIPEPSAQQLREIFNMARDQRSRATLYA |
| | | | | LSHAVNNYHKTNPTGTQELLDIANYLMEQIQDD |
| | | | | CTGDEDYTYLILRVIGNMGQTMEQLTPELKSSIL |
| | | | | KCVQSTKPSLMIQKAAIQALRKMEPKDKDQEVL |
| | | | | LQTFLDDASPGDKRLAAYLMLMRSPSQADINKI |
| | | | | VQILPWEQNEQVKNFVASHIANILNSEELDIQDL |
| | | | | KKLVKEALKESQLPTVMDFRKFSRNYQLYKSVS |
| | | | | LPSLDPASAKIEGNLIFDPNNYLPKESMLKTTLTA |
| | | | | FGFASADLIEIGLEGKGFEPTLEALFGKQGFFPDS |
| | | | | VNKALYWVNGQVPDGVSKVLVDHFGYTKDDK |
| | | | | HEQDMVNGIMLSVEKLIKDLKSKEVPEARAYLRI |
| | | | | LGEELGFASLHDLQLLGKLLLMGARTLQGIPQMI |
| | | | | GEVIRKGSKNDFFLHYIFMENAFELPTGAGLQLQ |
| | | | | ISSSGVIAPGAKAGVKLEVANMQAELVAKPSVS |
| | | | | VEFVTNMGIIIPDFARSGVQMNTNFFHESGLEAH |
| | | | | VALKAGKLKFIIPSPKRPVKLLSGGNTLHLVSTTK |
| | | | | TEVIPPLIENRQSWSVCKQVFPGLNYCTSGAYSN |
| | | | | ASSTDSASYYPLTGDTRLELELRPTGEIEQYSVSA |
| | | | | TYELQREDRALVDTLKFVTQAEGAKQTEATMTF |
| | | | | KYNRQSMTLSSEVQIPDFDVDLGTILRVNDESTE |
| | | | | GKTSYRLTLDIQNKKITEVALMGHLSCDTKEERK |
| | | | | IKGVISIPRLQAEARSEILAHWSPAKLLLQMDSSA |
| | | | | TAYGSTVSKRVAWHYDEEKIEFEWNTGTNVDT |
| | | | | KKMTSNFPVDLSDYPKSLHMYANRLLDHRVPQT |
| | | | | DMTFRHVGSKLIVAMSSWLQKASGSLPYTQTLQ |
| | | | | DHLNSLKEFNLQNMGLPDFHIPENLFLKSDGRVK |
| | | | | YTLNKNSLKIEIPLPFGGKSSRDLKMLETVRTPAL |
| | | | | HFKSVGFHLPSREFQVPTFTIPKLYQLQVPLLGVL |
| | | | | DLSTNVYSNLYNWSASYSGGNTSTDHFSLRARY |
| | | | | HMKADSVVDLLSYNVQGSGETTYDHKNTFTLSY |
| | | | | DGSLRHKFLDSNIKFSHVEKLGNNPVSKGLLIFD |
| | | | | ASSSWGPQMSASVHLDSKKKQHLFVKEVKIDGQ |
| | | | | FRVSSFYAKGTYGLSCQRDPNTGRLNGESNLRFN |
| | | | | SSYLQGTNQITGRYEDGTLSLTSTSDLQSGIIKNT |
| | | | | ASLKYENYELTLKSDTNGKYKNFATSNKMDMT |
| | | | | FSKQNALLRSEYQADYESLRFFSLLSGSLNSHGL |
| | | | | ELNADILGTDKINSGAHKATLRIGQDGISTSATTN |
| | | | | LKCSLLVLENELNAELGLSGASMKLTTNGRFRE |
| | | | | HNAKFSLDGKAALTELSLGSAYQAMILGVDSKN |
| | | | | IFNFKVSQEGLKLSNDMMGSYAEMKFDHTNSLN |
| | | | | IAGLSLDFSSKLDNIYSSDKFYKQTVNLQLQPYSL |
| | | | | VTTLNSDLKYNALDLTNNGKLRLEPLKLHVAGN |
| | | | | LKGAYQNNEIKHIYAISSAALSASYKADTVAKVQ |
| | | | | GVEFSHRLNTDIAGLASAIDMSTNYNSDSLHFSN |
| | | | | VFRSVMAPFTMTIDAHTNGNGKLALWGEHTGQ |
| | | | | LYSKFLLKAEPLAFTFSHDYKGSTSHHLVSRKSIS |
| | | | | AALEHKVSALLTPAEQTGTWKLKTQFNNNEYSQ |
| | | | | DLDAYNTKDKIGVELTGRTLADLTLLDSPIKVPL |
| | | | | LLSEPINIIDALEMRDAVEKPQEFTIVAFVKYDKN |
| | | | | QDVHSINLPFFETLQEYFERNRQTIIVVLENVQRN |
| | | | | LKHINIDQFVRKYRAALGKLPQQANDYLNSFNW |
| | | | | ERQVSHAKEKLTALTKKYRITENDIQIALDDAKI |
| | | | | NFNEKLSQLQTYMIQFDQYIKDSYDLHDLKIAIA |
| | | | | NIIDEIIEKLKSLDEHYHIRVNLVKTIHDLHLFIENI |
| | | | | DFNKSGSSTASWIQNVDTKYQIRIQIQEKLQQLK |
| | | | | RHIQNIDIQHLAGKLKQHIEAIDVRVLLDQLGTTI |
| | | | | SFERINDILEHVKHFVINLIGDFEVAEKINAFRAK |
| | | | | VHELIERYEVDQQIQVLMDKLVELAHQYKLKETI |
| | | | | QKLSNVLQQVKIKDYFEKLVGFIDDAVKKLNEL |
| | | | | SFKTFIEDVNKFLDMLIKKLKSFDYHQFVDETND |
| | | | | KIREVTQRLNGEIQALELPQKAEALKLFLEETKA |
| | | | | TVAVYLESLQDTKITLIINWLQEALSSASLAHMK |
| | | | | AKFRETLEDTRDRMYQMDIQQELQRYLSLVGQV |
| | | | | YSTLVTYISDWWTLAAKNLTDFAEQYSIQDWAK |
| | | | | RMKALVEQGFTVPEIKTILGTMPAFEVSLQALQK |
| | | | | ATFQTPDFIVPLTDLRIPSVQINFKDLKNIKIPSRFS |
| | | | | TPEFTILNTFHIPSFTIDFVEMKVKIIRTIDQMLNSE |
| | | | | LQWPVPDIYLRDLKVEDIPLARITLPDFRLPEIAIP |
| | | | | EFIIPTLNLNDFQVPDLHIPEFQLPHISHTIEVPTFG |
| | | | | KLYSILKIQSPLFTLDANADIGNGTTSANEAGIAA |
| | | | | SITAKGESKLEVLNFDFQANAQLSNPKINPLALK |
| | | | | ESVKFSSKYLRTEHGSEMLFFGNAIEGKSNTVAS |
| | | | | LHTEKNTLELSNGVIVKINNQLTLDSNTKYFHKL |
| | | | | NIPKLDFSSQADLRNEIKTLLKAGHIAWTSSGKG |
| | | | | SWKWACPRFSDEGTHESQISFTIEGPLTSFGLSNK |
| | | | | INSKHLRVNQNLVYESGSLNFSKLEIQSQVDSQH |
| | | | | VGHSVLTAKGMALFGEGKAEFTGRHDAHLNGK |
| | | | | VIGTLKNSLFFSAQPFEITASTNNEGNLKVRFPLR |
| | | | | LTGKIDFLNNYALFLSPSAQQASWQVSARFNQY |
| | | | | KYNQNFSAGNNENIMEAHVGINGEANLDFLNIPL |
| | | | | TIPEMRLPYTIITTPPLKDFSLWEKTGLKEFLKTT |
| | | | | KQSFDLSVKAQYKKNKHRHSITNPLAVLCEFISQ |
| | | | | SIKSFDRHFEKNRNNALDFVTKSYNETKIKFDKY |
| | | | | KAEKSHDELPRTFQIPGYTVPVVNVEVSPFTIEMS |
| | | | | AFGYVFPKAVSMPSFSILGSDVRVPSYTLILPSLE |
| | | | | LPVLHVPRNLKLSLPDFKELCTISHIFIPAMGNITY |
| | | | | DFSFKSSVITLNTNAELFNQSDIVAHLLSSSSSVID |
| | | | | ALQYKLEGTTRLTRKRGLKLATALSLSNKFVEGS |
| | | | | HNSTVSLTTKNMEVSVATTTKAQIPILRMNFKQE |
| | | | | LNGNTKSKPTVSSSMEFKYDFNSSMLYSTAKGA |
| | | | | VDHKLSLESLTSYFSIESSTKGDVKGSVLSREYSG |
| | | | | TIASEANTYLNSKSTRSSVKLQGTSKIDDIWNLEV |
| | | | | KENFAGEATLQRIYSLWEHSTKNHLQLEGLFFTN |
| | | | | GEHTSKATLELSPWQMSALVQVHASQPSSFHDF |
| | | | | PDLGQEVALNANTKNQKIRWKNEVRIHSGSFQS |
| | | | | QVELSNDQEKAHLDIAGSLEGHLRFLKNIILPVY |
| | | | | DKSLWDFLKLDVTTSIGRRQHLRVSTAFVYTKN |
| | | | | PNGYSFSIPVKVLADKFIIPGLKLNDLNSVLVMPT |
| | | | | FHVPFTDLQVPSCKLDFREIQIYKKLRTSSFALNL |
| | | | | PTLPEVKFPEVDVLTKYSQPEDSLIPFFEITVPESQ |
| | | | | LTVSQFTLPKSVSDGIAALDLNAVANKIADFELP |
| | | | | TIIVPEQTIEIPSIKFSVPAGIVIPSFQALTARFEVDS |
| | | | | PVYNATWSASLKNKADYVETVLDSTCSSTVQFL |
| | | | | EYELNVLGTHKIEDGTLASKTKGTFAHRDFSAEY |
| | | | | EEDGKYEGLQEWEGKAHLNIKSPAFTDLHLRYQ |
| | | | | KDKKGISTSAASPAVGTVGMDMDEDDDFSKWN |
| | | | | FYYSPQSSPDKKLTIFKTELRVRESDEETQIKVNW |
| | | | | EEEAASGLLTSLKDNVPKATGVLYDYVNKYHW |
| | | | | EHTGLTLREVSSKLRRNLQNNAEWVYQGAIRQI |
| | | | | DDIDVRFQKAASGTTGTYQEWKDKAQNLYQEL |
| | | | | LTQEGQASFQGLKDNVFDGLVRVTQEFHMKVK |
| | | | | HLIDSLIDFLNFPRFQFPGKPGIYTREELCTMFIRE |
| | | | | VGTVLSQVYSKVHNGSEILFSYFQDLVITLPFELR |
| | | | | KHKLIDVISMYRELLKDLSKEAQEVFKAIQSLKT |
| | | | | TEVLRNLQDLLQFIFQLIEDNIKQLKEMKFTYLIN |
| | | | | YIQDEINTIFSDYIPYVFKLLKENLCLNLHKFNEFI |
| | | | | QNELQEASQELQQIHQYIMALREEYFDPSIVGWT |
| | | | | VKYYELEEKIVSLIKNLLVALKDFHSEYIVSASNF |
| | | | | TSQLSSQVEQFLHRNIQEYLSILTDPDGKGKEKIA |
| | | | | ELSATAQEIIKSQAIATKKIISDYHQQFRYKLQDF |
| | | | | SDQLSDYYEKFIAESKRLIDLSIQNYHTFLIYITEL |
| | | | | LKKLQSTTVMNPYMKLAPGELTIIL |
| 644 | HPT | Haptoglobin | P00738 | MSALGAVIALLLWGQLFAVDSGNDVTDIADDGC |
| | | | | PKPPEIAHGYVEHSVRYQCKNYYKLRTEGDGVY |
| | | | | TLNDKKQWINKAVGDKLPECEADDGCPKPPEIA |
| | | | | HGYVEHSVRYQCKNYYKLRTEGDGVYTLNNEK |
| | | | | QWINKAVGDKLPECEAVCGKPKNPANPVQRILG |
| | | | | GHLDAKGSFPWQAKMVSHHNLTTGATLINEQW |
| | | | | LLTTAKNLFLNHSENATAKDIAPTLTLYVGKKQL |
| | | | | VEIEKVVLHPNYSQVDIGLIKLKQKVSVNERVMP |
| | | | | ICLPSKDYAEVGRVGYVSGWGRNANFKFTDHLK |
| | | | | YVMLPVADQDQCIRHYEGSTVPEKKTPKSPVGV |
| | | | | QPILNEHTFCAGMSKYQEDTCYGDAGSAFAVHD |
| | | | | LEEDTWYATGILSFDKSCAVAEYGVYVKVTSIQ |
| | | | | DWVQKTIAEN |
| 645 | HPT | Haptoglobin | P00738 | MSALGAVIALLLWGQLFAVDSGNDVTDIADDGC |
| | | | | PKPPEIAHGYVEHSVRYQCKNYYKLRTEGDGVY |
| | | | | TLNDKKQWINKAVGDKLPECEADDGCPKPPEIA |
| | | | | HGYVEHSVRYQCKNYYKLRTEGDGVYTLNNEK |
| | | | | QWINKAVGDKLPECEAVCGKPKNPANPVQRILG |
| | | | | GHLDAKGSFPWQAKMVSHHNLTTGATLINEQW |
| | | | | LLTTAKNLFLNHSENATAKDIAPTLTLYVGKKQL |
| | | | | VEIEKVVLHPNYSQVDIGLIKLKQKVSVNERVMP |
| | | | | ICLPSKDYAEVGRVGYVSGWGRNANFKFTDHLK |
| | | | | YVMLPVADQDQCIRHYEGSTVPEKKTPKSPVGV |
| | | | | QPILNEHTFCAGMSKYQEDTCYGDAGSAFAVHD |
| | | | | LEEDTWYATGILSFDKSCAVAEYGVYVKVTSIQ |
| | | | | DWVQKTIAEN |
| 646 | A1AT | Alpha-1- | P01009 | MPSSVSWGILLLAGLCCLVPVSLAEDPQGDAAQ |
| | | antitrypsin | | KTDTSHHDQDHPTFNKITPNLAEFAFSLYRQLAH |
| | | | | QSNSTNIFFSPVSIATAFAMLSLGTKADTHDEILE |
| | | | | GLNFNLTEIPEAQIHEGFQELLRTLNQPDSQLQLT |
| | | | | TGNGLFLSEGLKLVDKFLEDVKKLYHSEAFTVN |
| | | | | FGDTEEAKKQINDYVEKGTQGKIVDLVKELDRD |
| | | | | TVFALVNYIFFKGKWERPFEVKDTEEEDFHVDQ |
| | | | | VTTVKVPMMKRLGMFNIQHCKKLSSWVLLMKY |
| | | | | LGNATAIFFLPDEGKLQHLENELTHDIITKFLENE |
| | | | | DRRSASLHLPKLSITGTYDLKSVLGQLGITKVFSN |
| | | | | GADLSGVTEEAPLKLSKAVHKAVLTIDEKGTEA |
| | | | | AGAMFLEAIPMSIPPEVKFNKPFVFLMIEQNTKSP |
| | | | | LFMGKVVNPTQK |
| 647 | IGM | Immunoglobulin | P01871 | GSASAPTLFPLVSCENSPSDTSSVAVGCLAQDFLP |
| | | heavy | | DSITFSWKYKNNSDISSTRGFPSVLRGGKYAATS |
| | | constant mu | | QVLLPSKDVMQGTDEHVVCKVQHPNGNKEKNV |
| | | | | PLPVIAELPPKVSVFVPPRDGFFGNPRKSKLICQA |
| | | | | TGFSPRQIQVSWLREGKQVGSGVTTDQVQAEAK |
| | | | | ESGPTTYKVTSTLTIKESDWLGQSMFTCRVDHRG |
| | | | | LTFQQNASSMCVPDQDTAIRVFAIPPSFASIFLTK |
| | | | | STKLTCLVTDLTTYDSVTISWTRQNGEAVKTHT |
| | | | | NISESHPNATFSAVGEASICEDDWNSGERFTCTV |
| | | | | THTDLPSPLKQTISRPKGVALHRPDVYLLPPARE |
| | | | | QLNLRESATITCLVTGFSPADVFVQWMQRGQPL |
| | | | | SPEKYVTSAPMPEPQAPGRYFAHSILTVSEEEWN |
| | | | | TGETYTCVVAHEALPNRVTERTVDKSTGKPTLY |
| | | | | NVSLVMSDTAGTCY |
| 648 | IC1 | Plasma protease | P05155 | MASRLTLLTLLLLLLAGDRASSNPNATSSSSQDP |
| | | C1 inhibitor | | ESLQDRGEGKVATTVISKMLFVEPILEVSSLPTTN |
| | | | | STTNSATKITANTTDEPTTQPTTEPTTQPTIQPTQP |
| | | | | TTQLPTDSPTQPTTGSFCPGPVTLCSDLESHSTEA |
| | | | | VLGDALVDFSLKLYHAFSAMKKVETNMAFSPFS |
| | | | | IASLLTQVLLGAGENTKTNLESILSYPKDFTCVHQ |
| | | | | ALKGFTTKGVTSVSQIFHSPDLAIRDTFVNASRTL |
| | | | | YSSSPRVLSNNSDANLELINTWVAKNTNNKISRL |
| | | | | LDSLPSDTRLVLLNAIYLSAKWKTTFDPKKTRME |
| | | | | PFHFKNSVIKVPMMNSKKYPVAHFIDQTLKAKV |
| | | | | GQLQLSHNLSLVILVPQNLKHRLEDMEQALSPSV |
| | | | | FKAIMEKLEMSKFQPTLLTLPRIKVTTSQDMLSI |
| | | | | MEKLEFFDFSYDLNLCGLTEDPDLQVSAMQHQT |
| | | | | VLELTETGVEAAAASAISVARTLLVFEVQQPFLF |
| | | | | VLWDQQHKFPVFMGRVYDPRA |
| 649 | APOH | Beta-2- | P02749 | MISPVLILFSSFLCHVAIAGRTCPKPDDLPFSTVVP |
| | | glycoprotein 1 | | LKTFYEPGEEITYSCKPGYVSRGGMRKFICPLTGL |
| | | | | WPINTLKCTPRVCPFAGILENGAVRYTTFEYPNTI |
| | | | | SFSCNTGFYLNGADSAKCTEEGKWSPELPVCAPII |
| | | | | CPPPSIPTFATLRVYKPSAGNNSLYRDTAVFECLP |
| | | | | QHAMFGNDTITCTTHGNWTKLPECREVKCPFPS |
| | | | | RPDNGFVNYPAKPTLYYKDKATFGCHDGYSLDG |
| | | | | PEEIECTKLGNWSAMPSCKASCKVPVKKATVVY |
| | | | | QGERVKIQEKFKNGMLHGDKVSFFCKNKEKKCS |
| | | | | YTEDAQCIDGTIEVPKCFKEHSSLAFWKTDASDV |
| | | | | KPC |
| 650 | FETUA | Alpha-2-HS- | P02765 | MKSLVLLLCLAQLWGCHSAPHGPGLIYRQPNCD |
| | | glycoprotein | | DPETEEAALVAIDYINQNLPWGYKHTLNQIDEVK |
| | | | | VWPQQPSGELFEIEIDTLETTCHVLDPTPVARCSV |
| | | | | RQLKEHAVEGDCDFQLLKLDGKFSVVYAKCDSS |
| | | | | PDSAEDVRKVCQDCPLLAPLNDTRVVHAAKAAL |
| | | | | AAFNAQNNGSNFQLEEISRAQLVPLPPSTYVEFT |
| | | | | VSGTDCVAKEATEAAKCNLLAEKQYGFCKATLS |
| | | | | EKLGGAEVAVTCMVFQTQPVSSQPQPEGANEAV |
| | | | | PTPVVDPDAPPSPPLGAPGLPPAGSPPDSHVLLAA |
| | | | | PPGHQLHRAHYDLRHTFMGVVSLGSPSGEVSHP |
| | | | | RKTRTVVQPSVGAAAGPVVPPCPGRIRHFKV |
| 651 | KLKB1 | Plasma | P03952 | MILFKQATYFISLFATVSCGCLTQLYENAFFRGG |
| | | Kallikrein | | DVASMYTPNAQYCQMRCTFHPRCLLFSFLPASSI |
| | | | | NDMEKRFGCFLKDSVTGTLPKVHRTGAVSGHSL |
| | | | | KQCGHQISACHRDIYKGVDMRGVNFNVSKVSSV |
| | | | | EECQKRCTNNIRCQFFSYATQTFHKAEYRNNCLL |
| | | | | KYSPGGTPTAIKVLSNVESGFSLKPCALSEIGCHM |
| | | | | NIFQHLAFSDVDVARVLTPDAFVCRTICTYHPNC |
| | | | | LFFTFYTNVWKIESQRNVCLLKTSESGTPSSSTPQ |
| | | | | ENTISGYSLLTCKRTLPEPCHSKIYPGVDFGGEEL |
| | | | | NVTFVKGVNVCQETCTKMIRCQFFTYSLLPEDC |
| | | | | KEEKCKCFLRLSMDGSPTRIAYGTQGSSGYSLRL |
| | | | | CNTGDNSVCTTKTSTRIVGGTNSSWGEWPWQVS |
| | | | | LQVKLTAQRHLCGGSLIGHQWVLTAAHCFDGLP |
| | | | | LQDVWRIYSGILNLSDITKDTPFSQIKEIIIHQNYK |
| | | | | VSEGNHDIALIKLQAPLNYTEFQKPICLPSKGDTS |
| | | | | TIYTNCWVTGWGFSKEKGEIQNILQKVNIPLVTN |
| | | | | EECQKRYQDYKITQRMVCAGYKEGGKDACKGD |
| 652 | VTNC | Vitronectin | P04004 | SGGPLVCKHNGMWRLVGITSWGEGCARREQPG |
| | | | | VYTKVAEYMDWILEKTQSSDGKAQMQSPA |
| | | | | MAPLRPLLILALLAWVALADQESCKGRCTEGEN |
| | | | | VDKKCQCDELCSYYQSCCTDYTAECKPQVTRGD |
| | | | | VFTMPEDEYTVYDDGEEKNNATVHEQVGGPSLT |
| | | | | SDLQAQSKGNPEQTPVLKPEEEAPAPEVGASKPE |
| | | | | GIDSRPETLHPGRPQPPAEEELCSGKPFDAFTDLK |
| | | | | NGSLFAFRGQYCYELDEKAVRPGYPKLIRDVWG |
| | | | | IEGPIDAAFTRINCQGKTYLFKGSQYWRFEDGVL |
| | | | | DPDYPRNISDGFDGIPDNVDAALALPAHSYSGRE |
| | | | | RVYFFKGKQYWEYQFQHQPSQEECEGSSLSAVF |
| | | | | EHFAMMQRDSWEDIFELLFWGRTSAGTRQPQFIS |
| | | | | RDWHGVPGQVDAAMAGRIYISGMAPRPSLAKK |
| | | | | QRFRHRNRKGYRSQRGHSRGRNQNSRRPSRAT |
| | | | | WLSLFSSEESNLGANNYDDYRMDWLVPATCEPI |
| | | | | QSVFFFSGDKYYRVNLRTRRVDTVDPPYPRSIAQ |
| | | | | YWLGCPAPGHL |

Table 63 identifies and defines the glycan structures from Table 55. Table 63 identifies a graphical representation of the structure and a coded representation of the composition for each glycan structure included in Table 55. As used herein, the 4-digit GL NO. is a designation that represents the number of hexoses, the number of HexNAcs, the number of Fucoses, and the number of Neuraminic Acids. Table 63 and Table 76 collectively identify and define the glycan structures from Table 58A.1 and Table 58B.1.

TABLE 63
Glycan Structure GL NOS: Structure and Composition

| Glycan Structure | | |
|---|---|---|
| GL NO. | Structure | Composition |
| 3310 | [symbol] | Hex(3)HexNAc(3)Fuc(1)NeuAc(0) |
| 4400 | [symbol] | Hex(4)HexNAc(4)Fuc(0)NeuAc(0) |
| 4410 | [symbol] | Hex(4)HexNAc(4)Fuc(1)NeuAc(0) |
| 4411 | [symbol] | Hex(4)HexNAc(4)Fuc(1)NeuAc(1) |
| 4500 | [symbol] | Hex(4)HexNAc(5)Fuc(0)NeuAc(0) |
| 5200 | [symbol] | Hex(5)HexNAc(2)Fuc(0)NeuAc(0) |
| 5400 | [symbol] | Hex(5)HexNAc(4)Fuc(0)NeuAc(0) |
| 5401 | [symbol] | Hex(5)HexNAc(4)Fuc(0)NeuAc(1) |
| 5402 | [symbol] | Hex(5)HexNAc(4)Fuc(0)NeuAc(2) |
| 5410 | [symbol] | Hex(5)HexNAc(4)Fuc(1)NeuAc(0) |
| 5411 | [symbol] | Hex(5)HexNAc(4)Fuc(1)NeuAc(1) |
| 5420 | [symbol] | Hex(5)HexNAc(4)Fuc(2)NeuAc(0) |
| 5421 | [symbol] | Hex(5)HexNAc(4)Fuc(2)NeuAc(1) |
| 5500 | [symbol] | Hex(5)HexNAc(5)Fuc(0)NeuAc(0) |
| 5501 | [symbol] | Hex(5)HexNAc(5)Fuc(0)NeuAc(1) |
| 5502 | [symbol] | Hex(5)HexNAc(5)Fuc(0)NeuAc(2) |
| 5510 | [symbol] | Hex(5)HexNAc(5)Fuc(1)NeuAc(0) |
| 5511 | [symbol] | Hex(5)HexNAc(5)Fuc(1)NeuAc(1) |
| 6200 | [symbol] | Hex(6)HexNAc(2)Fuc(0)NeuAc(0) |
| 6301 | [symbol] | Hex(6)HexNAc(3)Fuc(0)NeuAc(1) |
| 6311 | [symbol] | Hex(6)HexNAc(3)Fuc(1)NeuAc(1) |
| 6501 | [symbol] | Hex(6)HexNAc(5)Fuc(0)NeuAc(1) |
| 6502 | [symbol] | Hex(6)HexNAc(5)Fuc(0)NeuAc(2) |
| 6503 | [symbol] | Hex(6)HexNAc(5)Fuc(0)NeuAc(3) |
| 6511 | [symbol] | Hex(6)HexNAc(5)Fuc(1)NeuAc(1) |
| 6520 | [symbol] | Hex(6)HexNAc(5)Fuc(2)NeuAc(0) |
| 6610 | [symbol] | Hex(6)HexNAc(6)Fuc(1)NeuAc(0) |
| 7602 | [symbol] | Hex(7)HexNAc(6)Fuc(0)NeuAc(2) |
| 7603 | [symbol] | Hex(7)HexNAc(6)Fuc(0)NeuAc(3) |
| 10803 (5401 & 5402) (two glycans on the same peptide) | [symbol] | Hex(5)HexNAc(4)Fuc(0)NeuAc(1) and Hex(5)HexNAc(4)Fuc(0)NeuAc(2) |

Legend for Table 63

| | | | | |
|---|---|---|---|---|
| ● | [symbol] | [symbol] | [symbol] | [symbol] |
| Glc | Gal | Man | Fuc | Neu5Ac |
| | ▪ | [symbol] | [symbol] | |
| | GlcNAc | GalNAc | ManNAc | |

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for treatment management of melanoma. A transition includes a precursor ion and at least one product ion grouping. As reviewed herein, the peptide structures in Table 55, as well as their corresponding precursor ion and product ion groupings (these ions having defined m/z ratios or m/z ratios that fall within the m/z ranges identified herein), can be used in mass spectrometry-based analyses to predict treatment response, select a treatment for administration, determine whether to alter a treatment plan or dosage, or a combination thereof.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2A. The alkylation procedure may be implemented in a manner similar to, for example, alkylation procedure 204 in FIG. 2A. The digestion procedure may be implemented in a manner similar to, for example, digestion procedure 206 in FIG. 2A.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 60A or an m/z ratio within an identified m/z ratio as provided in Table 60A. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine-learning. In certain embodiments, the reaction monitoring mass spectrometry system may include multiple/selected reaction monitoring mass spectrometry (MRM/SRM-MS) to detect the one or more product ions and generate the quantification data.

### IX. Representative Experimental Results

### IX.A. Sample Preparation and Mass Spectrometry Data Production

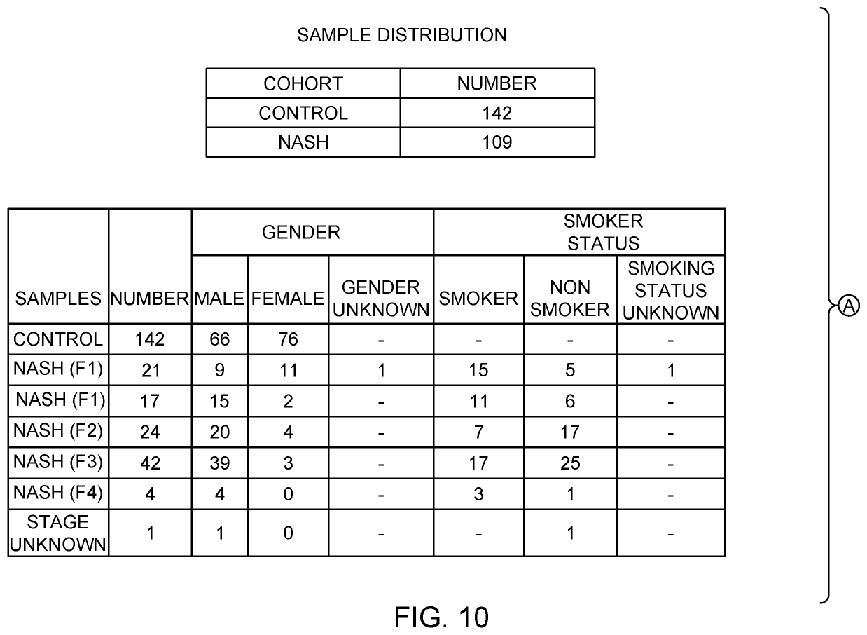

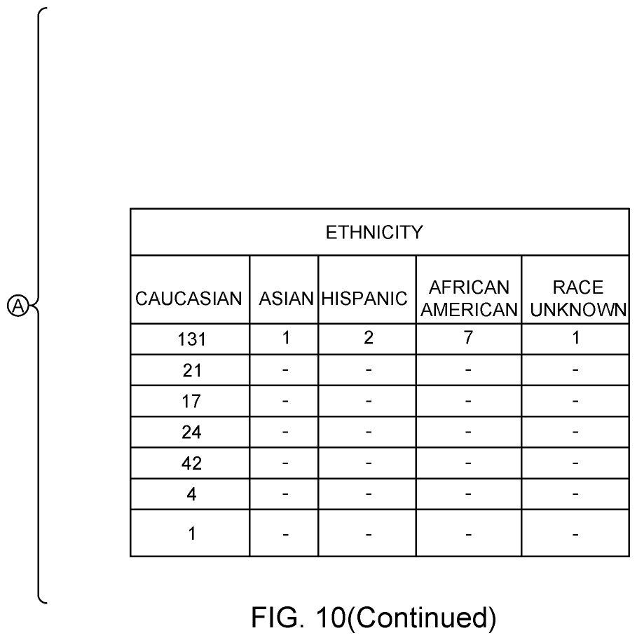

FIG. 10 is a table of the sample population used for the studies in accordance with various embodiments. The samples used to generate the experimental results included serum samples from 109 patients with a biopsy-proven diagnosis of NASH (88 biological male, 20 biological female, 1 biological sex unknown; Indivumed AG, Hamburg, Germany and from Bay Biosciences, Brookline, Massachusetts), and 142 healthy subjects with no history of liver disease (control, 66 male, 76 female) which were sourced from Precision for Medicine (Carlsbad, CA). In at least some cases, clinical diagnoses of patients with NASH were based on information provided by the sample supplier.

Prior to analysis, serum samples were reduced with DTT and alkylated with IAA followed by digestion with trypsin in a water bath at 37° C. for 18 hours. To quench the digestion, formic acid was added to each sample after incubation to a final concentration of 1%.

Digested serum samples were injected into a triple quadrupole mass spectrometer (MS) using a liquid chromatography system (e.g., a high-performance liquid chromatography (HPLC) system). Separation of the peptide structures (glycosylated and aglycosylated) was performed using a 70-min binary gradient. The triple quadrupole MS was operated in dynamic multiple reaction monitoring (dMRM) mode. Samples were injected in a randomized fashion with regard to underlying phenotype, and reference pooled serum digests were injected interspersed with study samples, at every 10^{th} sample position throughout the run.

An MRM analysis was performed on the peptide structures, representing a total of 48 high-abundance serum glycoproteins. A transition list consisted of glycopeptide structures as well as aglycosylated peptide structures from each glycoprotein. The python library Scikit-learn (https://scikit-leam.org/stable/) was used for statistical analyses and for building machine learning models.

Normalized abundance data was generated for the peptide structures using the following formula:

$$Normalized abundance for a given peptide structure = ( relative abundance of a peptide structure in sample / average relative abundance of the same peptide in the flanking reference serum samples$$

Relative abundance was calculated as the ratio of the raw abundance of any given peptide in a sample to the raw abundance of an aglycosylated peptide from the same glycoprotein in the sample.

### IX.B. Analysis of Peptide Structure Data (e.g., Abundance Data) and Confirming their Diagnostic Power

FIG. 9 illustrates that peptide structures for a single glycoprotein (e.g., A1AT, A2MG, AGP1, AAACT, AGP12, etc.) as well as peptide structures for a combination of such glycoproteins may be useful in diagnosing when a subject has progressed from a non-NASH (e.g., healthy state) to NASH or from early stage NASH to late stage NASH (not shown).

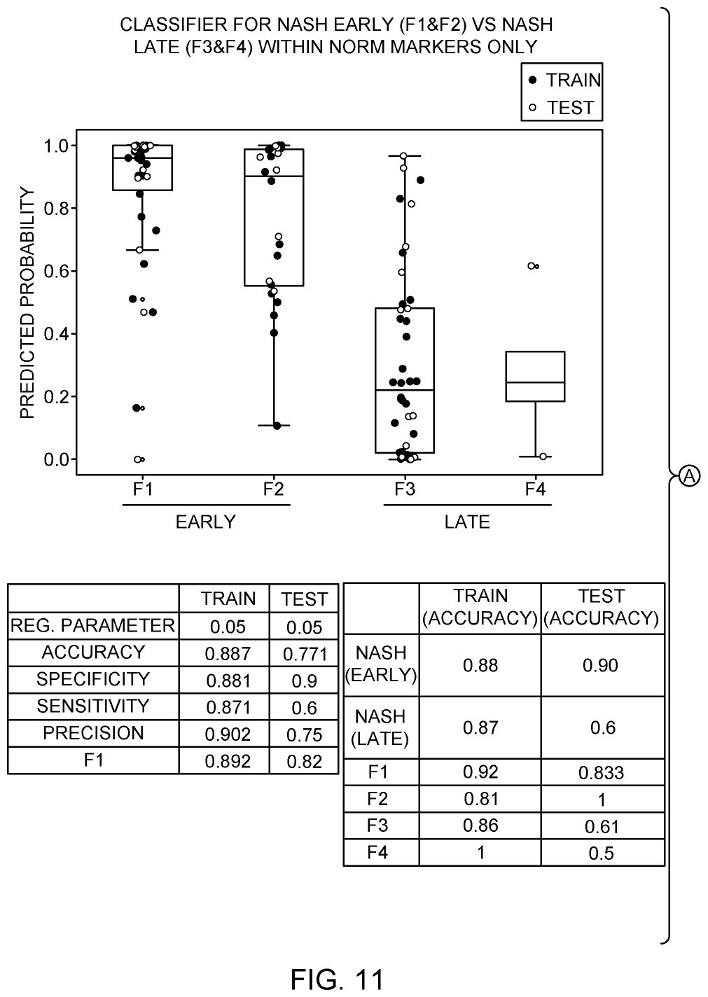

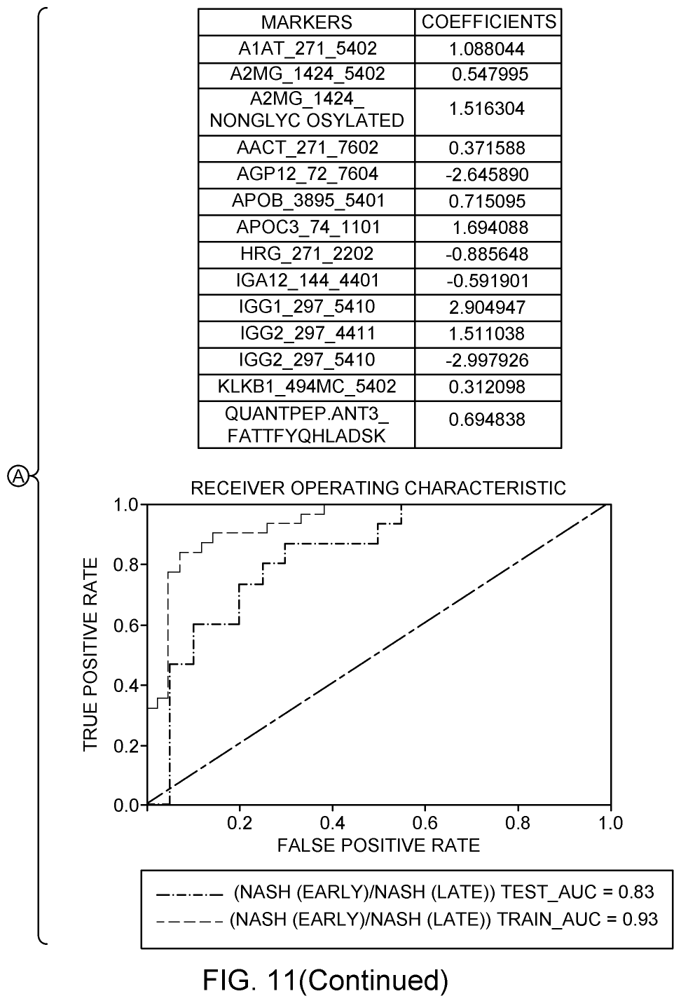

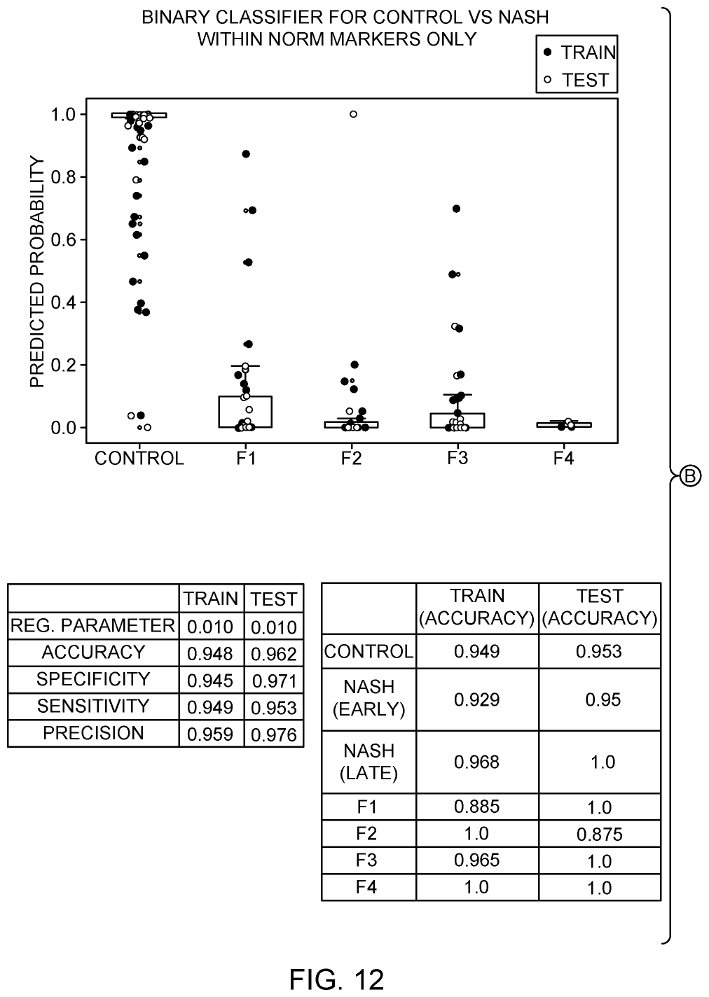

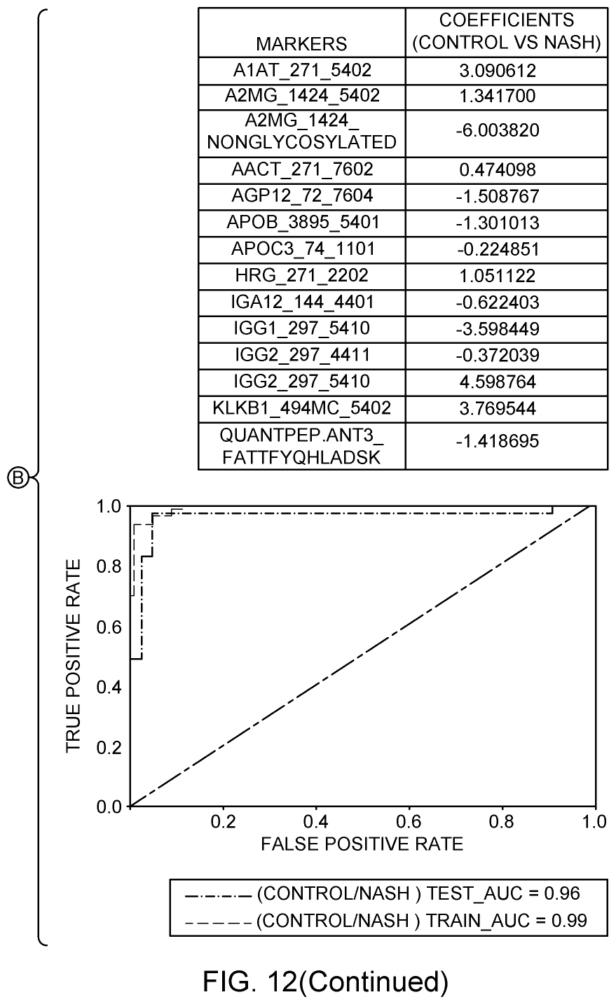

The normalized abundances of various peptide structures (e.g., those peptide structures identified in Table 1B) were used to train a first regression model (e.g., Model 1) to generate a disease indicator for a subject. The disease indicator was generated as a score (e.g., probability score) in which the range in which the score falls enables diagnosis or classification as non-NASH (e.g., control) to NASH (FIG. 12). The normalized abundances of various peptide structures (e.g., those peptide structures identified in Table 1B) were used to train a first regression model (e.g., Model 2) to generate a disease indicator for a subject. The disease indicator was generated as a score (e.g., probability score) in which the range in which the score falls enables diagnosis or classification as an early stage NASH state or a late stage NASH state (FIG. 11).

### IX.C. Validation

The upper left panel of FIG. 11 is a plot diagram illustrating validation of the disease indicator's ability to distinguish between early stage NASH state and late stage NASH, in accordance with various embodiments. As depicted, a disease indicator of about 0.5-1 was generally accurate in classifying as an early stage NASH state and a disease indicator of about 0.0-0.5 was generally accurate in classifying as a late stage NASH state. The upper right panel of FIG. 11 is a plot diagram of the receiver-operating-characteristic (ROC) curve for distinguishing between the early stage NASH state and the late stage NASH state for both the training and testing sets in accordance with various embodiments. A 70:30 train:test split of the training data was utilized, in which 70% of the training data was used to train a logistic regression model, which was used to make a prediction on the remaining 30% of the data. As shown in the panel, the area under the ROC curve (AUROC) for the training set was found to be 0.93, while the AUROC for the testing set was found to be 0.83.

The upper left panel of FIG. 12 is a plot diagram illustrating validation of the disease indicator's ability to distinguish between a control state and NASH, in accordance with various embodiments. As depicted, a disease indicator of about 0.5-1.0 was generally accurate in classifying as a control (e.g., non-NASH individual) and a disease indicator of about 0.0-0.5 was generally accurate in classifying as a NASH state.

The upper right panel of FIG. 12 is a plot diagram of the receiver-operating-characteristic (ROC) curve for distinguishing between a healthy control state and a NASH state for both the training and testing sets in accordance with various embodiments. Again, a 70:30 train:test split of the training data was utilized, in which 70% of the training data was used to train a logistic regression model, which was used to make a prediction on the remaining 30% of the data. As shown in the panel, the area under the ROC curve (AUROC) for the training set was found to be 0.99, while the AUROC for the testing set was found to be 0.96.

### IX.C.1. Validation using 3 Representative Patients

Three representative patients were tested using a trained regression model trained to distinguish between NASH and control. Table 9 provides the normalized abundances determined for each patient for various peptide structures. At the bottom of Table 9, the disease indicators computed for these patients based on the normalized abundances are provided. The disease indicator is a probability score that indicates the likelihood that the subject has NASH. For model 1, a predicted probability >0.5 indicates there may be a likelihood that the sample is a control or from an individual without NASH, and a predicted probability ≤0.5 indicates there may be a likelihood that the individual has NASH. In the present example, all three patients were correctly identified.

TABLE 9
Patient Examples

| | Patient 1 | Patient 2 | Patient 3 |
|---|---|---|---|
| | Probabil- | Probabil- | Probabil- |
| Com- | ity | ity | ity |
| pound | Score | Score | Score |
| Name | (Model 1) | (Model 1) | (Model 1) |
| A1AT_ | 1.346131747 | 1.134416126 | 1.289330789 |
| 271_ | | | |
| 5402 | | | |
| A2MG_ | 0.984693144 | 1.334874883 | 1.175643546 |
| 1424_ | | | |
| 5402 | | | |
| A2MG_ | 1.149145341 | 0.955301313 | 1.126117245 |
| 1424_ | | | |
| NONGLY- | | | |
| COSY- | | | |
| LATED | | | |
| AACT_ | 0.581364042 | 0.481911284 | 0.855041665 |
| 271_ | | | |
| 7602 | | | |
| AGP12_ | 1.559377174 | 0.992967622 | 0.770675162 |
| 72_ | | | |
| 7604 | | | |
| APOB_ | 0.980588067 | 1.222024624 | 1.267639338 |
| 3895_ | | | |
| 5401 | | | |
| APOC3_ | 1.114566801 | 0.849938341 | 1.108545626 |
| 74_ | | | |
| 1101 | | | |
| HRG_ | 0.558787554 | 0.96031758 | 0.746959247 |
| 271_ | | | |
| 2202 | | | |
| IGA12_ | 0.830701312 | 1.114278084 | 0.453645283 |
| 144_ | | | |
| 4401 | | | |
| IGG1_ | 0.708349304 | 0.597807186 | 1.183152656 |
| 297_ | | | |
| 5410 | | | |
| IGG2_ | 0.935383107 | 0.827287795 | 1.032551293 |
| 297_ | | | |
| 4411 | | | |
| IGG2_ | 0.772837034 | 0.46381311 | 1.14166881 |
| 297_ | | | |
| 5410 | | | |
| KLKB1_ | 0.780186994 | 1.59389123 | 1.608936811 |
| 494MC_ | | | |
| 5402 | | | |
| QUANT- | 1.223800501 | 1.023398181 | 1.160593734 |
| PEP. | | | |
| ANT3_ | | | |
| FATTFY | | | |
| QHLADSK | | | |
| Model 1 | 0.923148544 | 0.099315721 | 0.320775355 |
| Pre- | | | |
| dicted | | | |
| Proba- | | | |
| bility | | | |
| Score | | | |

In some embodiments, samples from one or more individuals are subjected to Model 2, in which case a predicted probability >0.5 indicates there may be a likelihood for the presence of early stage NASH and ≤0.5 indicates a likelihood for the presence of late stage NASH.

### X. Exemplary Embodiments for NASH Identification and Fibrosis Stage Prediction

The present disclosure concerns embodiments for systems, methods, and compositions related to identification of non-alcoholic steatohepatitis (NASH) and/or identification or prediction of a fibrosis stage in an individual with NASH. The embodiments concern classifying biological samples, measuring for one or more certain markers from a biological sample, assaying for one or more certain markers from a biological sample, determining the presence of one or more certain markers from a biological sample, and so forth. The embodiments of the disclosure utilize models that accurately either identify that an individual may have NASH or that identify the fibrosis stage of NASH of the individual based on the presence of one or more markers in sample(s) from the individual. In various embodiments, a model identifies that the individual has NASH and another model identifies the particular fibrotic stage of the NASH of the individual.

In particular embodiments, the systems, methods, and compositions encompassed in the disclosure are significantly different to distinguish stages of fibrosis in NASH despite the glycoprotein profile being similar among at least some of the NASH stages. The systems, methods, and compositions encompassed herein also are sufficiently specific to utilize markers that distinguish between control and NASH. In various embodiments, the systems, methods, and compositions encompassed herein identify progression from the absence of NASH to early stage NASH to late stage NASH. In various embodiments, there may be fewer marker differences between early stage and late stage NASH compared to more marker differences between control or absence of NASH and NASH. In some embodiments, the markers are accurate regardless of the status of one or more characteristics of the individual: biological sex, sample source, sample collection, smoker status, or age.

In various embodiments of the disclosure, an individual is in need of identifying whether or not they have NASH or whether or not they have early stage fibrosis of NASH or late stage fibrosis of NASH. The individual may be in need of such identification based on family history of fatty liver disease and/or based on having one or more symptoms of NASH, such as fatigue and/or upper right abdomen pain, although the individual may be asymptomatic in the early stages. The individual may be in need of identifying whether or not they have NASH and, upon confirmation of NASH, they may be in need of identifying the stage of fibrosis of NASH. In some cases, the same sample from the individual is utilized for both tests, although in other cases it is not. The same markers may or may not be utilized for both tests. In some cases, the analysis of the sample of the individual is the sole test utilized for identifying NASH and/or fibrosis stage, whereas in other cases a medical provider may utilize one or more other tests, such as ultrasound, magnetic resonance imaging, transient elastography, ultrasound elastography, and/or magnetic resonance elastography.

Any individual may be subject to methods of the disclosure, including any person of any biological sex, any gender, any smoker status, any ethnicity, and so forth. In various embodiments, an individual is subject to any method encompassed herein as a part of routine preventative or health check medical practices or because NASH is present or suspected of being present.

In various embodiments, the sample for analysis for NASH identification and/or fibrosis staging is not a liver biopsy. In particular embodiments, the sample for analysis for NASH identification and/or fibrosis staging is serum from the individual. The present disclosure provides for measuring for one or more circulating glycoproteins, glycopeptides, or non-glycosylated peptides in serum to diagnose or identify the presence of NASH and/or to predict stages of fibrosis in individuals diagnosed with NASH. In various embodiments, the sample is measured for 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the peptides of Table 1A. In various embodiments, the sample is measured for 1, 2, 3, 4, 5, 6, 7, 8, 9, 10, 11, 12, 13, or all of the peptides of Table 1B.

Embodiments of the disclosure include methods of classifying samples, including serum samples, from an individual suspected of having, known to have, or at risk for having non-alcoholic steatohepatitis (NASH) by measuring from the sample for one or more glycopeptides and/or non-glycosylated peptides encompassed herein. The methods encompass whether or not NASH is identified in the individual. In some cases, the measuring identifies the individual as not having NASH or as having NASH. In various embodiments, in cases wherein the individual has one or more glycopeptides and/or non-glycosylated peptides of Table 1A or 1, or certain levels thereof compared to control or healthy individuals, the individual may be determined to have NASH. In various embodiments, in cases wherein the individual lacks the glycopeptides and/or non-glycosylated peptides of Table 1A or 1B, or has certain levels thereof compared to control or healthy individuals, the individual may be determined not to have NASH. The measuring may identify the individual as having a particular stage of fibrosis of NASH, including at least early stage or late stage. In some cases, one or more samples from the individual are measured for one or more glycopeptides and/or non-glycosylated peptides encompassed herein that identify the individual as having a particular stage of fibrosis of NASH that is early stage or late stage. In specific cases, the measuring comprises successive or concomitant steps of identifying that the individual has NASH and that the individual has a particular stage of fibrosis of NASH.

In various embodiments, an individual at risk for having NASH is subjected to methods of the disclosure to identify, or not, the presence of NASH. Such methods also measure for one or more glycopeptides and/or non-glycosylated peptides encompassed herein. In various embodiments, in cases wherein the individual has one or more glycopeptides and/or non-glycosylated peptides of Table 1A or 1, the individual may be determined to have NASH. In various embodiments, in cases wherein the individual lacks the glycopeptides and/or non-glycosylated peptides of Table 1A or 1, the individual may be determined not to have NASH. The individual may be of any kind, although in specific cases individual at risk for having NASH has a family history of fatty liver disease; diabetes; obesity; metabolic syndrome; dyslipidemia; hypertension; elevated levels of Aspartate aminotransferase and/or alanine transaminase; a mutation in patatin-like phospholipase domain-containing 3 (PNPLA3); a mutation in transmembrane 6 superfamily member 2 (TM6SF2); a mutation in membrane bound O-acyltransferase domain-containing 7 (MBOAT7); a mutation in glucokinase regulator (GCKR), or a combination thereof.

Embodiments of the disclosure include methods of predicting a stage of fibrosis in NASH in an individual in need thereof by measuring for one or more glycopeptides or non-glycosylated peptides from Table 1A or 1B in one or more samples, which may comprise serum, from the individual. The individual may be known to have NASH or may be suspected of having NAS The predicted stage may be early stage or late stage fibrosis. In various embodiments, the sample is measured for 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the peptides of Table 1A. In various embodiments, the sample is measured for 1, 2, 3, 4, 5, 6, 7, 8, 9, 10, 11, 12, 13, or all of the peptides of Table 1B.

In embodiments wherein the measuring identifies the individual as having NASH, the individual may be recommended to take action to treat NASH, slow the progression of NASH, or (particularly in the case of early stage NASH) reverse one or more symptoms of NASH. In particular embodiments, an individual may be recommended to lose weight, exercise more, control glucose level, reduce cholesterol, limit salt intake, limit sugar intake, reduce cholesterol, avoid or reduce alcohol intake, avoid liver-harming medications or dietary supplements, get vaccinated for hepatitis A, get vaccinated for hepatitis B, take vitamin E, take pioglitazone, take liraglutide, monitor for hepatic cell carcinoma (HCC), or a combination thereof. In various embodiments, the individual with NASH is at risk for developing HCC, and the individual may be tested for HCC and/or begin monitoring periodically for the presence of HCC.

### XI. Exemplary Methodologies Relating to Diagnosis Based on Peptide Structure Data Analysis

### XI.A. General Methodology

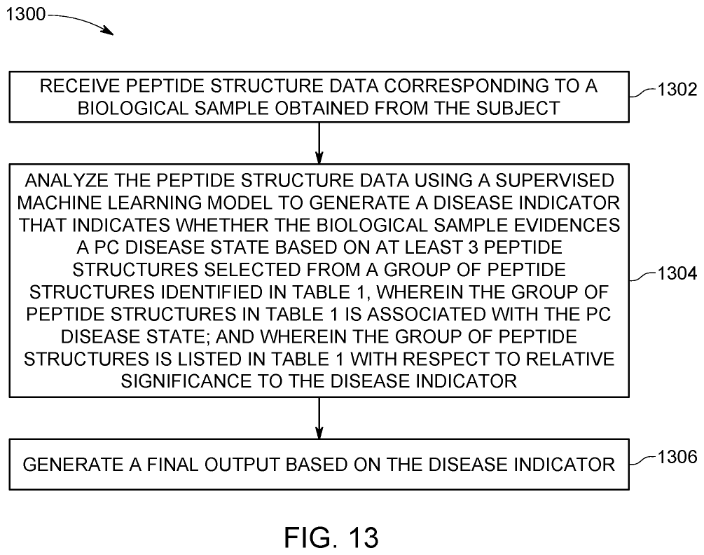

FIG. 13 is a flowchart of a process for diagnosing a subject with respect to a breast cancer (BC) disease state in accordance with one or more embodiments. Process 1300 may be implemented using, for example, at least a portion of workflow 100 as described in FIGS. 1, 2A, and 2B and/or analysis system 300 as described in FIG. 3. Process 1300 may be used to generate a final output that includes at least a diagnosis output for the subject.

Step 1302 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 9, with the peptide sequence being one of SEQ ID NOS: 46-62 as defined in Table 9.

Step 1304 includes analyzing the peptide structure data using a supervised machine learning model to generate a disease indicator that indicates whether the biological sample evidences a BC disease state based on at least 1 peptide structure selected from a group of peptide structures identified in Table 9, Table 10A, or Table 10B (below). In step 504, the group of peptide structures in Table 9, Table 10A, or Table 10B is associated with the BC disease state. The group of peptide structures is listed in Table 9, Table 10A, or Table 10B with respect to relative significance to the disease indicator.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, or all 18 of the peptide structures PS-30 through PS-47 in Table 9.

In one or more embodiments, the at least 1 peptide structure includes at least 1, at least 2, at least 3, at least 4, at least 5, at least 6, or all 7 of the peptide structures PS-33, PS-42, PS-44, PS-30, PS-47, PS-43, or PS-37 in Table 10A.

In one or more embodiments, the at least 1 peptide structure includes at least 1, at least 2, at least 3, at least 4, at least 5, at least 6, at least 7, or all 8 of the peptide structures PS-42, PS-33, PS-41, PS-43, PS-47, PS-37, PS-30, or PS-45 in Table 10B.

TABLE 9
Peptide Structures associated with Breast Cancer

| | | | | Linking | Linking | | |
|---|---|---|---|---|---|---|---|
| | | | | Site | Site | | |
| | | Prot | Pept | Position | Position | Glycan | |
| | | SEQ | SEQ | within | within | Struc | Mono- |
| PS-ID | | ID | ID | Prot | Pept | GL | isotopic |
| NO. | PS-NAME | NO. | NO. | Seq | Seq | NO. | mass |
| 33 | A2MG_1424_ | 63 | 46 | 247 | 10 | 5402 | 4635.973824 |
| | NONGLYCOSYLATED | | | | | | |
| 44 | THRB_416_5401 | 64 | 47 | 416 | 1 | 5401 | 3133.296654 |
| 30 | A1AT_107_6502 | 65 | 48 | 107 | 14 | 6502 | 6260.721064 |
| 42 | PLASMAFGB_ | 66 | 49 | N/A | N/A | N/A | 1949.995878 |
| | EEAPSLRPAPPPISGGGYR | | | | | | |
| 43 | QUANTPEP-APOM_ | 67 | 50 | N/A | N/A | N/A | 816.485756 |
| | AFLLTPR | | | | | | |
| 37 | C1S_174_5402 | 68 | 51 | 174 | 5 | 5402 | 5730.401612 |
| 47 | VTNC_242_5412 | 69 | 52 | 242 | 1 | 5412 | 5122.145318 |
| 45 | THRB_416MC_5401 | 64 | 53 | 416 | 17 | 5401 | 5084.278074 |
| 41 | IGJ_71MC_ | 70 | 54 | N/A | N/A | N/A | 2147.169814 |
| | NONGLYCOSYLATED | | | | | | |
| 34 | AACT_127_5401 | 71 | 55 | 127 | 3 | 5401 | 4125.727334 |
| 32 | A2MG_1424_5402_Z5 | 63 | 56 | 1424 | 3 | 5402 | 4366.945898 |
| 38 | CFAH_1095_5401 | 72 | 57 | 1095 | 17 | 5401 | 4930.910914 |
| 31 | A2MG_1424_5402_Z3 | 63 | 56 | 1424 | 3 | 5402 | 4366.945898 |
| 39 | HRG_271_2202 | 73 | 58 | 271 | 1 | 2202 | 2838.255284 |
| 40 | IGA2_205_5410 | 74 | 59 | 205 | 6 | 5410 | 2726.188952 |
| 35 | ANT_128_5402 | 75 | 60 | 128 | 5 | 5402 | 4070.673904 |
| 46 | TRFE_432_ | 76 | 61 | N/A | N/A | N/A | 1475.744216 |
| | NONGLYCOSYLATED | | | | | | |
| 36 | APOM_135_5402 | 67 | 62 | 135 | 15 | 5402 | 4735.914404 |

TABLE 10A
Peptide Structures for Model 1: Healthy vs. Breast Cancer

| | | Protein | Peptide |
|---|---|---|---|
| PS-ID | | SEQ ID | SEQ ID |
| NO. | PS-NAME | NO. | NO. |
| 33 | A2MG_1424_NONGLYCOSYLATED | 63 | 46 |
| 42 | PLASMAFGB_EEAPSLRPAPPPISGGGYR | 66 | 49 |
| 44 | THRB_416_5401 | 64 | 47 |
| 30 | A1AT_107_6502 | 65 | 48 |
| 47 | VTNC_242_5412 | 69 | 52 |
| 43 | QUANTPEP-APOM_AFLLTPR | 67 | 50 |
| 37 | C1S_174_5402 | 68 | 51 |

TABLE 10B
Peptide Structures for Model 2: Healthy vs. Early Stage Breast
Cancer

| | | Protein | Peptide |
|---|---|---|---|
| PS-ID | | SEQ ID | SEQ ID |
| NO. | PS-NAME | NO. | NO. |
| 42 | PLASMAFGB_EEAPSLRPAPPPISGGGYR | 66 | 49 |
| 33 | A2MG_1424_NONGLYCOSYLATED | 63 | 46 |
| 41 | IGJ_71MC_NONGLYCOSYLATED | 70 | 54 |
| 43 | QUANTPEP-APOM_AFLLTPR | 67 | 50 |
| 47 | VTNC_242_5412 | 69 | 52 |
| 37 | C1S_174_5402 | 68 | 51 |
| 30 | A1AT_107_6502 | 65 | 48 |
| 45 | THRB_416MC_5401 | 64 | 53 |

In one or more embodiments, step 1304 may be implemented using a binary classification model (e.g., a regression model). In some examples, the regression model may be, for example, penalized multivariable regression model. In various embodiments, the disease indicator may be computed using a weight coefficient associated with each peptide structure of the at least 1 peptide structure, the weight coefficient of a corresponding peptide structure of the at least 1 peptide structure may indicate the relative significance of the corresponding peptide structure to the disease indicator.

In some embodiments, step 1304 may include computing a peptide structure profile for the biological sample that identifies a weighted value for each peptide structure of the at least 1 peptide structure. The weighted value for a peptide structure of the at least 1 peptide structure may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is positive for the BC disease state and the supervised machine learning model is configured to generate an output that identifies the biological sample as either evidencing (“positive for”) the BC disease state when the disease indicator is greater than a selected threshold or not evidencing (“negative for”) the BC disease state when the disease indicator is not greater than the selected threshold. The selected threshold may be, for example, 0.30, 0.35, 0.40, 0.45, 0.50, 0.55, or some other threshold. In one or more embodiments, the selected threshold is 0.5.

Step 1306 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the BC disease state if the biological sample evidences the BC disease state based on the disease indicator. The diagnosis may be, for example, “negative” if the biological sample does not evidence the BC disease state based on the disease indicator. A negative diagnosis may mean that the biological sample has a non-breast cancer (BC) state (e.g., healthy, control, etc.). The negative diagnosis for the BC disease state can include at least one of a healthy state or a control state.

Generating the diagnosis output in step 1306 may include determining that the score falls above a selected threshold and generating a positive diagnosis for the BC disease state. Alternatively, step 1306 can include determining that the score falls below a selected threshold and generating a negative diagnosis for the BC disease state. In some scoring systems, the score can include a probability score and the selected threshold can be 0.5. In other scoring systems, the selected threshold can fall within a range between 0.4 and 0.6.

In one or more embodiments, the final output in step 1306 may include a treatment output if the diagnosis output indicates a positive diagnosis for the BC disease state. The treatment output may include, for example, at least one of an identification of a treatment for the subject, a treatment plan for administering the treatment, or both. Treatment for breast cancer may include, for example, but is not limited to, at least one of radiation therapy, chemoradiotherapy, surgery, a targeted drug therapy, or some other form of treatment. The treatment plan may include, for example, but is not limited to, a timeline or schedule for administering the treatment, dosing information, other treatment-related information, or a combination thereof.

### XI. B. Training the Model to Diagnose with respect to the BC Disease State

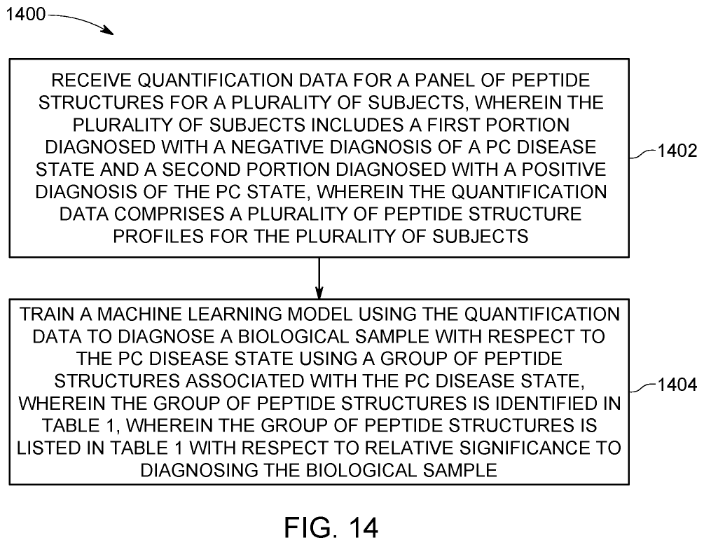

FIG. 14 is a flowchart of a process for training a model to diagnose a subject with respect to a breast cancer (BC) disease state in accordance with one or more embodiments. Process 1400 may be implemented using, for example, at least a portion of workflow 100 as described in FIGS. 1, 2A, and 2B and/or analysis system 300 as described in FIG. 3. In some embodiments, process 1400 may be one example of an implementation for training the model used in the process 1300 in FIG. 13.

Step 1402 includes receiving quantification data for a panel of peptide structures for a plurality of subjects. The plurality of subjects includes a first portion diagnosed with a negative diagnosis of a BC disease state and a second portion diagnosed with a positive diagnosis of the BC disease state. The quantification data comprises a plurality of peptide structure profiles for the plurality of subjects.

Step 1404 includes training a machine learning model using the quantification data to diagnose a biological sample with respect to the BC disease state using a group of peptide structures associated with the BC disease state (e.g., the group of peptide structures is identified in Table 9). The group of peptide structures is listed in Table 9 with respect to relative significance to diagnosing the biological sample. Step 1404 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the BC disease state and a negative diagnosis for any subject of the plurality of subjects determined not to have the BC disease state.

The machine learning model can include a binary classification model. Some binary classification models can include logistical regression models. Some logistical regression models can include LASSO regression models.

An alternative or additional step in process 1400 can include performing a differential expression analysis using initial training data to compare a first portion of the plurality of subjects diagnosed with the positive diagnosis for the BC disease state versus a second portion of the plurality of subjects diagnosed with the negative diagnosis for the BC disease state.

An alternative or additional step in process 1400 can include identifying a training group of peptide structures based on the differential expression analysis for use as prognostic markers for the BC disease state.

An alternative or additional step in process 1400 can include forming the training data based on the training group of peptide structures identified.

An alternative or additional step in process 1400 can include identifying a training group of peptide structures based on the differential expression analysis, wherein the training group of peptide structures is a subset of the plurality of peptide structures relevant to diagnosing the BC disease state. The subset may be identified based on at least one of fold-changes, false discovery rates, or p-values computed as part of the differential expression analysis.

An alternative or additional step in process 1400 can include training a machine learning model, using the quantification data for the training group of peptide structures, to diagnose a subject of a biological sample with respect to the BC disease state using a group of peptide structures associated with the BC disease state. The group of peptide structures may be a subset of the training group of peptide structures and is identified in Table 9. The group of peptide structures is listed in Table 9 with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures of Table 10A or 10B below, which include at least a portion of the group of peptide structures identified in Table 9. The markers used for training of the LASSO regression model may, in one or more embodiments, additionally include one or more other peptide structure markers.

In one or more embodiments, a subset of the markers identified in Table 10A or 10B may be used for training of the LASSO regression model. Alternatively, the markers identified in Table 10A or 10B may be a subset for training of the LASSO regression model. For example, the LASSO regression model may be trained using at least one other marker in addition to those identified in Table 10A or 10B.

### XI.C. Monitoring a Subject for a Breast Cancer Disease State

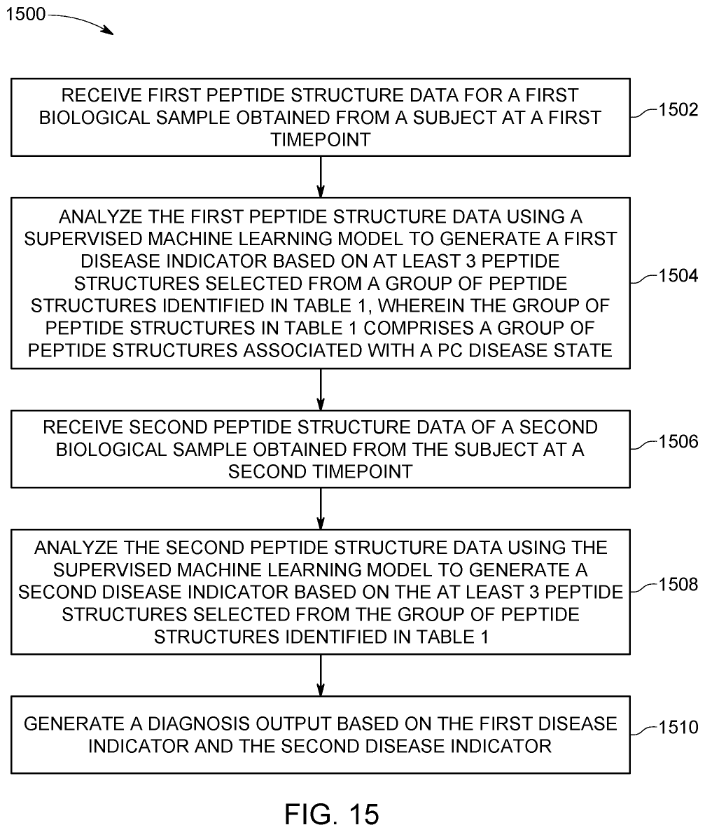

FIG. 15 is a flowchart of a process for monitoring a subject for a breast cancer (BC) disease state in accordance with one or more embodiments. Process 1500 may be implemented using, for example, at least a portion of workflow 100 as described in FIGS. 1, 2A, and 2B and/or analysis system 300 as described in FIG. 3.

Step 1502 includes receiving first peptide structure data for a first biological sample obtained from a subject at a first timepoint.

Step 1504 includes analyzing the first peptide structure data using a supervised machine learning model to generate a first disease indicator based on at least 1 peptide structure selected from a group of peptide structures identified in Table 9. The group of peptide structures in Table 9 includes a group of peptide structures associated with a BC disease state in accordance with various embodiments. The supervised machine can be a binary classification model. In some embodiments, the binary classification model can be a logistical regression model.

Step 1506 includes receiving second peptide structure data of a second biological sample obtained from the subject at a second timepoint.

Step 1508 includes analyzing the second peptide structure data using the supervised machine learning model to generate a second disease indicator based on the at least 1 peptide structure selected from the group of peptide structures identified in Table 9.

Step 1510 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the BC disease state and the second biological sample evidences the positive diagnosis for the BC disease. In other embodiments, the diagnosis output identifies whether a non-BC disease state has progressed to the BC disease state, wherein the non-BC disease state includes either a healthy state, a control state, or a benign pancreatitis state.

### XI.D. Peptide Structure and Product Ion Compositions, Kits and Reagents

Aspects of the disclosure include compositions comprising one or more of the peptide structures listed in Table 9. In some embodiments, a composition comprises a plurality of the peptide structures listed in Table 9. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, or all of the peptide structures listed in Table 9. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 46-62, listed in Table 9.

Aspects of the disclosure include compositions comprising one or more precursor ions having a defined charge and/or defined mass-to-charge (m/z) ratio, as listed in Table 11. Aspects of the disclosure include compositions comprising one or more product ions having a defined mass-to-charge (m/z) ratio, which product ions are produced by converting a peptide structure described herein (e.g., a peptide structure listed in Table 9) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 9). In some embodiments, a composition comprises a set of the product ions listed in Table 11, having an m/z ratio selected from the list provided for each peptide structure in Table 9 or Table 11.

In some embodiments, a composition comprises at least one of peptide structures PS-30 through PS-47 identified in Table 9. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, or all 18 of the peptide structures PS-30 through PS-47 in Table 9.

In one or more embodiments, a composition comprises at least 1, at least 2, at least 3, at least 4, at least 5, at least 6, or all 7 of the peptide structures PS-33, PS-42, PS-44, PS-30, PS-47, PS-43, or PS-37 in Table 10A.

In one or more embodiments, a composition comprises at least 1, at least 2, at least 3, at least 4, at least 5, at least 6, at least 7, or all 8 of the peptide structures PS-42, PS-33, PS-41, PS-43, PS-47, PS-37, PS-30, or PS-45 in Table 10B.

In some embodiments, a composition comprises a peptide structure or a product ion. The peptide structure or product ion comprises an amino acid sequence having at least 90% sequence identity to any one of SEQ ID NOS: 46-62, as identified in Table 12, corresponding to peptide structures PS-30 through PS-47 in Table 9.

In some embodiments, a composition comprises a peptide structure or a product ion. The peptide structure or product ion comprises an amino acid sequence having at least 90% sequence identity to any one of SEQ ID NOS: 46-62 as identified in Table 12, corresponding to peptide structures PS-30 through PS-47 in Table 9.

In some embodiments, the product ion is selected as one from a group consisting of product ions identified in Table 11, including product ions falling within an identified m/z range of the m/z ratio identified in Table 11 and characterized as having a precursor ion having an m/z ratio within an identified m/z range of the m/z ratio identified in Table 11. A first range for the product ion m/z ratio may be ±0.5. A second range for the product ion m/z ratio may be ±0.8. A third range for the production m/z ratio may be ±1.0. A first range for the precursor ion m/z ratio may be 1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 11, and characterized as having a precursor ion having an m/z ratio that falls within at least one of first range (±0.5), a second range (±1.0), or a third range (±1.0 of the precursor ion m/z ratio identified in Table 11.

TABLE 11
Mass Spectrometry-Related Characteristics for the
Peptide Structures associated with Breast Cancer

| PS- | Mono- | | | | 2nd | | |
|---|---|---|---|---|---|---|---|
| ID | isotopic | Precursor | Precursor. | Product | Prod | RT | Collision. |
| NO. | mass | m/z | charge | m/z | m/z | (min) | Energy |
| 33 | 4635.973824 | 1160.5 | 4 | 366.1 | N/A | 40.665 | 20 |
| 44 | 3133.296654 | 1046.1 | 3 | 366.1 | N/A | 22.675 | 25 |
| 30 | 6260.721064 | 1253.6 | 5 | 366.1 | N/A | 42.8 | 30 |
| 42 | 1949.995878 | 651.01 | 3 | 811.44 | N/A | 21.1 | 166 |
| 43 | 816.485756 | 409.2 | 2 | 599.4 | N/A | 23.2 | 10 |
| 37 | 5730.401612 | 1147.8 | 5 | 366.1 | 366.1 | 25 | 41 |
| 47 | 5122.145318 | 1282 | 4 | 366.1 | 1488.2 | 37.4 | 38 |
| 45 | 5084.278074 | 1272.6 | 4 | 366.1 | N/A | 39.9 | 25 |
| 41 | 2147.169814 | 717.2 | 3 | 912 | N/A | 26.8 | 20 |
| 34 | 4125.727334 | 1032.9 | 4 | 366.1 | N/A | 33 | 20 |
| 32 | 4366.945898 | 874.4 | 5 | 366.1 | N/A | 44 | 15 |
| 38 | 4930.910914 | 1234.2 | 4 | 366.1 | N/A | 36.2 | 20 |
| 31 | 4366.945898 | 1457.3 | 3 | 366.1 | N/A | 44 | 30 |
| 39 | 2838.255284 | 710.8 | 4 | 274.1 | N/A | 6.6 | 15 |
| 40 | 2726.188952 | 909.8 | 3 | 366.1 | N/A | 12.2 | 22 |
| 35 | 4070.673904 | 1019.2 | 4 | 366.1 | N/A | 40.8 | 20 |
| 46 | 1475.744216 | 739 | 2 | 1047.55 | N/A | 28.3 | 20 |
| 36 | 4735.914404 | 1185.5 | 4 | 366.1 | N/A | 29.8 | 25 |

Table 12A defines the peptide sequences for SEQ TD NOS: 46-62 from Table 9. Table 12 further identifies a corresponding protein SEQ ID NO. for each peptide sequence.

TABLE 12A
Peptide SEQ ID NOS

| | | Corresponding |
|---|---|---|
| Peptide | | Protein |
| SEQ ID NO. | Peptide.sequence | SEQ ID NO. |
| 46 | IITILEEEMNVSVCGLYTYGK | 63 |
| 47 | NFTENDLLVR | 64 |
| 48 | ADTHDEILEGLNFNLTEIPEAQIHEGFQELLR | 65 |
| 49 | EEAPSLRPAPPPISGGGYR | 66 |
| 50 | AFLLTPR | 67 |
| 51 | NCGVNCSGDVFTALIGEIASPNYPKPYPENSR | 68 |
| 52 | NISDGFDGIPDNVDAALALPAHSYSGR | 69 |
| 53 | WVLTAAHCLLYPPWDKNFTENDLLVR | 64 |
| 54 | IIVPLNNRENISDPTSPLR | 70 |
| 55 | TLNQSSDELQLSMGNAMFVK | 71 |
| 56 | VSNQTLSLFFTVLQDVPVR | 63 |
| 57 | SPYEMFGDEEVMCLNGNWTEPPQCK | 72 |
| 58 | SSTTKPPFKPHGSR | 73 |
| 59 | TPLTANITK | 74 |
| 60 | LGACNDTLQQLMEVFK | 75 |
| 61 | CGLVPVLAENYNK | 76 |
| 62 | TELFSSSCPGGIMLNETGQGYQR | 67 |

Table 12B provides an indication of particular markers and includes the starting position of the peptide sequence within the protein sequence and the end position of the peptide sequence within the protein sequence.

TABLE 12B
Markers and Protein Positions

| PS-ID | | | Start | End |
|---|---|---|---|---|
| NO | PS-NAME | Peptide.sequence | position | position |
| 33 | A2MG_1424_ | IITILEEEMNVSVCGLYTYGK | 238 | 258 |
| | NONGLYCOSYLATED | | | |
| 44 | THRB_416_5401 | NFTENDLLVR | 416 | 425 |
| 30 | A1AT_107_6502 | ADTHDEILEGLNFNLTEIPEAQI | 94 | 125 |
| | | HEGFQELLR | | |
| 42 | PLASMAFGB_ | EEAPSLRPAPPPISGGGYR | 54 | 72 |
| | EEAPSLRPAPPPISGGGYR | | | |
| 43 | QUANTPEP- | AFLLTPR | 172 | 178 |
| | APOM_AFLLTPR | | | |
| 37 | C1S_174_5402 | NCGVNCSGDVFTALIGEIASPN | 170 | 201 |
| | | YPKPYPENSR | | |
| 47 | VTNC_242_5412 | NISDGFDGIPDNVDAALALPAH | 242 | 268 |
| | | SYSGR | | |
| 45 | THRB_416MC_5401 | WVLTAAHCLLYPPWDKNFTEN | 400 | 425 |
| | | DLLVR | | |
| 41 | IGJ_71MC_ | IIVPLNNRENISDPTSPLR | 62 | 80 |
| | NONGLYCOSYLATED | | | |
| 34 | AACT_127_5401 | TLNQSSDELQLSMGNAMFVK | 125 | 144 |
| 32 | A2MG_1424_5402_Z5 | VSNQTLSLFFTVLQDVPVR | 1422 | 1440 |
| 38 | CFAH_1095_5401 | SPYEMFGDEEVMCLNGNWTEP | 1079 | 1103 |
| | | PQCK | | |
| 31 | A2MG_1424_5402_Z3 | VSNQTLSLFFTVLQDVPVR | 1422 | 1440 |
| 39 | HRG_271_2202 | SSTTKPPFKPHGSR | 271 | 284 |
| 40 | IGA2_205_5410 | TPLTANITK | 200 | 208 |
| 35 | ANT_128_5402 | LGACNDTLQQLMEVFK | 124 | 139 |
| 46 | TRFE_432_ | CGLVPVLAENYNK | 421 | 433 |
| | NONGLYCOSYLATED | | | |
| 36 | APOM_135_5402 | TELFSSSCPGGIMLNETGQGYQ | 121 | 143 |
| | | R | | |

Table 13 identifies the proteins of SEQ TD NOS: 63-76 from Table 9. Table 13 identifies a corresponding protein abbreviation and protein name for each of protein SEQ TD NOS: 63-76. Further, Table 13 identifies a corresponding Uniprot TD for each of protein SEQ TD NOS: 63-76.

TABLE 13
Protein SEQ ID NOS

| Protein | | | Protein |
|---|---|---|---|
| Abbre- | | Uniprot | SEQ ID |
| viation | Protein Name | ID | NO. |
| A2MG | Alpha-2-macroglobulin | P01023 | 63 |
| THRB | Prothrombin | P00734 | 64 |
| A1AT | Alpha-1-antitrypsin | P01009 | 65 |
| PLASMAFGB | Fibrinogen beta chain | P02675 | 66 |
| APOM | Apolipoprotein M | O95445 | 67 |
| C1S | Complement C1s subcomponent | P09871 | 68 |
| VTNC | Vitronectin | P04004 | 69 |
| IGJ | Immunoglobulin J chain | P01591 | 70 |
| AACT | Alpha-1-antichymotrypsin | P01011 | 71 |
| CFAH1 | ComplementFactorH | P08603 | 72 |
| HRG | Histidine-rich Glycoprotein | P04196 | 73 |
| IGA2 | Immunoglobulin heavy constant | P01877 | 74 |
| | alpha 2 | | |
| ANT | Antithrombin-III | P01008 | 75 |
| TRFE | Serotransferrin | P02787 | 76 |

Table 14 identifies and defines the glycan structures included in Table 9, all of which are N-glycans. Table 14 identifies a coded representation of the composition for each glycan structure included in Table 9. As used herein, the 4-digit GL NO. is a designation that represents the number of hexoses, the number of HexNAcs, the number of Fucoses, and the number of Neuraminic Acids.

TABLE 14
Glycan Structure GL NOS: Composition

| Composition | Structure | Glycan composition | Glycan Mass |
|---|---|---|---|
| 5401 | [symbol] | Hex(5)HexNAc(4)Fuc(0)NeuAc(1) | 1931.687482 |
| 6502 | [symbol] | Hex(6)HexNAc(5)Fuc(0)NeuAc(2) | 2587.91508 |
| 5402 | [symbol] | Hex(5)HexNAc(4)Fuc(0)NeuAc(2) | 2222.782892 |
| 5412 | [symbol] | Hex(5)HexNAc(4)Fuc(1)NeuAc(2) | 2368.840798 |

Legend for Table 14:

| | | | | |
|---|---|---|---|---|
| ● | [symbol] | [symbol] | [symbol] | [symbol] |
| Glc | Gal | Man | Fuc | Neu5Ac |
| ▪ | [symbol] | [symbol] | [symbol] | [symbol] |
| GlcNAc | GalNAc | ManNAc | Xyl | Neu5Gc |
| [symbol] | [symbol] | [symbol] | | [symbol] |
| GlcN | GalN | ManN | | KdN |
| [symbol] | [symbol] | [symbol] | [symbol] | |
| GlcA | GalA | ManA | IdoA | |

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating a BC disease state. A transition includes a precursor ion and at least one product ion grouping. As reviewed herein, the peptide structures in Table 9, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, BC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 11 or an m/z ratio within an identified m/z ratio as provided in Table 11. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### XI.E. Representative Experimental Results

### Subject Sample Models & Corresponding Training of Said Models

To assess the association of individual peptide structures (biomarkers) with breast cancer, two differential expression analyses (DEAs) were run on three different subject cohorts, adjusting for age and sex. For Model 1, differential expression was performed to compare healthy controls vs all stages of breast cancer after adjusting for age. For Model 2, differential expression was performed (adjusting for age) to compare healthy controls with early stage breast cancer (stage 1 and stage 2). The markers to be used as input for the regularized logistic regression were selected based on the false discovery rate(FDR) cut off of 0.05.

The regularized logistic regression method LASSO was utilized for breast cancer disease classification and to identify key markers. LASSO is a type of logistic regression that uses L1 regularization, which adds a penalty equal to the absolute value of the magnitude of coefficients. This type of regularization can result in sparse models with few coefficients; some coefficients can become zero and eliminated from the model which helps us retain only the key coefficients useful for prediction. Here, 70% of the total dataset was used to train the model and tune hyperparameters. In alternative embodiments, other classification algorithms such as random forest, elastic net regression, generalized additive models, etc., can be used for classifying BC.

During training, a model validation technique called 5-fold cross validation was also performed that assists in assessing how the model will generalize to an independent dataset. The best performing model was ultimately determined based on highest accuracy score on the fewest markers that did not achieve substantial overfitting. This model is then run through an independent test set that consisted of 30% of the total set to confirm biomarker relevance and appropriate model performance.

For all of the analyses, concentration normalization was used. The formula for concentration normalization is provided below:

$$Peptide concentration = [ peptide abundance / corresponding ISTD abundance ] * spike - in concentration * dilution factor$$

$$GP concentration = [ GP abundance / glycopeptide ISTD abundance ] * spike - in concentration * dilution factor$$

After raw abundance is obtained, the raw abundance is adjusted by multiplying it by the ratio of the mean of Sigma serum samples in the patient run and the mean of the Sigma serum samples in a separate reference set. This result is considered the adjusted raw abundance. From then, the concentration normalization as described above is utilized to prepare the data for analysis.

The model will output a probability score per patient based on the glycoproteomic signatures identified. Because there are two models, each of them utilizes slightly different glycoproteomic profiles to make the diagnosis. For Model 1 (healthy vs all stages of breast cancer), in various embodiments the probability score may be used to label samples as “likely to develop breast cancer” vs “not likely to develop breast cancer” agnostic of stage. For Model 2 (healthy vs early stage breast cancer (stage 1 and stage 2)), in various embodiments the probability score may determine between healthy state vs early stage breast cancer in women.

In various embodiments, a novel platform was applied for characterizing blood glycoproteomic biomarkers, combining liquid-chromatography/mass spectrometry (LC-MS) with artificial intelligence/neural networks (AI-NN) to analyze serum samples from 279 breast cancer patients at various stages (median age 56 years; samples acquired from Bay Biosciences and iSpecimen) and to analyze 102 healthy control samples (median age 52 years; samples acquired from Precision for Medicine). A panel of 596 serum glycosylated and non-glycosylated peptides, representing 71 serum proteins, were analyzed. Age-adjusted differential expression analysis for 596 normalized biomarkers were performed to evaluate statistically significant differential abundances using an FDR-adjusted q-value of 0.05 as a cutoff. Using the top 243 differentially expressed markers as input, a LASSO penalized logistic regression model with 5 fold repeated cross validation was applied to identify the top biomarkers contributing to the separation between healthy controls and breast cancer patients.

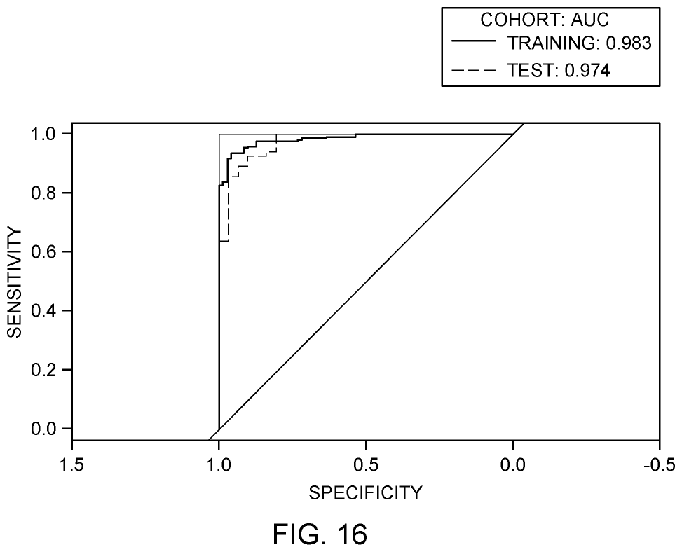

FIG. 16 is an example plot of a receiver operating characteristic (ROC) curve for Model 1 for the training set and testing set in accordance with one or more embodiments. The plot illustrates specificity versus sensitivity. Thus, out of 596 markers, 243 markers were identified that were differentially expressed (FDR≤0.05) between breast cancer samples and healthy controls. Out of those, 8 markers were obtained as the top predictors of classifying breast cancer patients and healthy controls from the LASSO classification algorithm that yielded an accuracy of 94% (93.8% sensitivity, 94.4% specificity) and the area under the curve (AUC) for the training set was 0.982. This classifier was validated on an independent test set with 30% of the subjects which gave an accuracy of 93% (95.2% sensitivity, 93.3% specificity) and AUC of 0.981.

TABLE 15A
First Differential Expression Analysis (DEA)

| | | | | Control | Control | Control |
|---|---|---|---|---|---|---|
| | | | | vs early | vs early | vs early |
| PS-ID | Control/BC | Control/BC | Control/BC | stage BC | stage BC | stage BC |
| NO. | (fold change) | (FDR) | (p-value) | (fold change) | (p-value) | (FDR) |
| 33 | 0.56 | 7.11E−46 | 1.41E−43 | 0.567 | 5.13E−40 | 1.02E−37 |
| 44 | 0.642 | 9.20E−16 | 7.83E−14 | 0.647 | 7.16E−13 | 4.74E−11 |
| 30 | 1.392 | 1.43E−12 | 7.08E−11 | 1.427 | 1.08E−12 | 5.87E−11 |
| 42 | 1.847 | 4.03E−10 | 1.00E−08 | 1.648 | 1.82E−06 | 1.81E−05 |
| 43 | 0.802 | 2.14E−13 | 1.42E−11 | 0.779 | 3.88E−14 | 3.31E−12 |
| 37 | 0.767 | 5.66E−13 | 3.07E−11 | 0.773 | 2.45E−11 | 7.70E−10 |
| 47 | 1.388 | 5.67E−11 | 1.88E−09 | 1.366 | 1.77E−08 | 3.01E−07 |
| 45 | 0.742 | 2.86E−12 | 1.22E−10 | 0.723 | 1.82E−12 | 9.05E−11 |
| 41 | 0.925 | 8.50E−12 | 3.38E−10 | 0.917 | 1.43E−11 | 5.11E−10 |
| 34 | 0.776 | 2.70E−12 | 1.22E−10 | 0.761 | 8.05E−13 | 4.80E−11 |
| 32 | 0.673 | 4.86E−11 | 1.81E−09 | 0.696 | 5.40E−09 | 1.04E−07 |
| 38 | 0.813 | 5.50E−11 | 1.88E−09 | 0.809 | 4.70E−10 | 1.08E−08 |
| 31 | 0.682 | 2.03E−10 | 6.05E−09 | 0.706 | 2.54E−08 | 4.09E−07 |
| 39 | 0.829 | 2.76E−10 | 7.48E−09 | 0.804 | 1.10E−11 | 4.39E−10 |
| 40 | 1.368 | 3.99E−10 | 1.00E−08 | 1.409 | 1.40E−09 | 2.88E−08 |
| 35 | 0.815 | 4.55E−10 | 1.08E−08 | 0.787 | 1.46E−11 | 5.11E−10 |
| 46 | 0.742 | 3.64E−09 | 7.23E−08 | 0.736 | 1.65E−07 | 2.14E−06 |
| 36 | 0.865 | 8.95E−09 | 1.67E−07 | 0.843 | 5.47E−10 | 1.21E−08 |

### A. Subject Sample Models

To assess the association of individual peptide structures (biomarkers) with pancreatic cancer, three differential expression analyses (DEAs) were run on three different subject cohorts, adjusting for age and sex.

Table 22 below identifies the fold changes, FDRs, and p-values as determined by the differential expression analysis (DEA) performed for the markers provided in Table 16. These DEA results yielded 25 markers that satisfied FDR 10¹² and concordance (AUC)≥0.7.

Model #1 Analysis: The subject cohort (Model #1), which corresponds to Model 1, for the first differential expression analysis included 290 subjects diagnosed with pancreatic cancer and 194 healthy control subjects. The samples for Model #1 was obtained from Precision for Medicine (healthy controls) and both Indivumed and iSpecimen for cancer samples. The fold change, FDR, and p-value information relevant to the markers for Model 1 can be identified by cross-referencing that information provided in Table 22 with the marker list of Table 17A.

Model #2 Analysis: The subject cohort (Model #2), which corresponds to Model 2, for the second differential expression analysis included 308 subjects diagnosed with pancreatic cancer and 259 subjects that were either healthy controls or diagnosed with benign pancreatitis. The pancreatic cancer samples for Model #2 were obtained from Indivumed, iSpecimen, and University of Iowa, the healthy controls were obtained from Precision for Medicine, and the samples for pancreatitis were obtained from both Indivumed and University of Iowa.

Model #3 Analysis: The subject cohort (Model #3), which corresponds to Model 3, for the third differential expression analysis included 205 subjects diagnosed with Stage 1 or Stage 2 pancreatic cancer and 259 subjects that were either healthy controls or diagnosed with benign pancreatitis. The early stage pancreatic cancer samples for Model #3 were obtained from Indivumed, University of Iowa, and iSpecimen, the healthy control samples were obtained from Precision for Medicine, and the benign pancreatitis samples were obtained from both Indivumed and University of Iowa.

This different differential expression analysis was run for various peptide structures (e.g., hundreds of different peptide structures) across the markers listed in Table 22 for comparing healthy control vs. pancreatic cancer disease state (i.e., Model 1). Table 22 provides the statistical results (e.g., false discovery rates (FDRs), fold changes, p-values) for this analysis for the 55 peptide structure markers identified in Table 16. These 55 peptide structure markers were determined to be highly relevant markers for diagnosing pancreatic cancer.

TABLE 22
First Differential Expression Analysis (DEA)

| | | Fold | | |
|---|---|---|---|---|
| | | change | P-value | FDR |
| | | (healthy vs | (healthy vs | (healthy vs |
| PS-NAME | PS-ID NO. | pancreatic) | pancreatic) | pancreatic) |
| A1AT_107_5412 | 48 | 1.178 | 5.22E-02 | 8.59E-02 |
| A1AT_107_6512 | 49 | 1.629 | 2.21E-09 | 1.53E-08 |
| A1AT_107_NONGLYCOSYLATED | 50 | 0.286 | 5.10E-14 | 9.04E-13 |
| A1AT_70_5402 | 51 | 0.785 | 3.32E-25 | 2.21E-23 |
| A1BG_179_5402 | 52 | 1.092 | 1.53E-05 | 5.31E-05 |
| A2MG_1424_NONGLYCOSYLATED | 53 | 0.846 | 8.76E-20 | 3.59E-18 |
| A2MG_247_5200 | 54 | 0.622 | 6.16E-14 | 1.06E-12 |
| A2MG_247_5401 | 55 | 0.809 | 1.16E-19 | 4.41E-18 |
| A2MG_247MC_5401 | 56 | 0.828 | 4.36E-16 | 1.10E-14 |
| A2MG_55_5412 | 57 | 0.877 | 6.28E-05 | 1.94E-04 |
| A2MG_869_5401 | 58 | 1.295 | 7.79E-26 | 5.92E-24 |
| A2MG_869_5402 | 59 | 1.341 | 5.32E-18 | 1.67E-16 |
| A2MG_869_6301 | 60 | 1.155 | 1.03E-15 | 2.49E-14 |
| AACT_271_6512 | 61 | 1.036 | 5.87E-01 | 6.61E-01 |
| AGP1_93_7614 | 62 | 1.35 | 1.94E-09 | 1.36E-08 |
| AGP12_56_5412 | 63 | 1.213 | 3.37E-05 | 1.11E-04 |
| AGP12_72_7601 | 64 | 0.919 | 1.35E-01 | 1.99E-01 |
| AGP12_72MC_6503 | 65 | 0.637 | 5.53E-23 | 3.08E-21 |
| APOB_3895_5401 | 66 | 0.757 | 1.81E-09 | 1.30E-08 |
| APOB_983_5402 | 67 | 1.286 | 9.29E-28 | 9.50E-26 |
| APOH_253_5412 | 68 | 1.697 | 3.31E-17 | 9.26E-16 |
| APOM_135_5401 | 69 | 1.033 | 2.19E-01 | 3.03E-01 |
| APOM_135_5402 | 70 | 1.163 | 3.91E-15 | 7.99E-14 |
| C1S_174_5402 | 71 | 0.815 | 2.40E-12 | 3.20E-11 |
| CERU_397_5412 | 72 | 1.298 | 4.00E-08 | 2.29E-07 |
| CO5_741_5401 | 73 | 0.961 | 8.67E-02 | 1.33E-01 |
| CO5_741_5402 | 74 | 0.9 | 7.47E-15 | 1.42E-13 |
| FETUA_156_5412 | 75 | 1.512 | 2.26E-15 | 4.80E-14 |
| HEMO_187_5412 | 76 | 1.232 | 1.50E-15 | 3.35E-14 |
| HEMO_453_5401 | 77 | 0.755 | 3.87E-19 | 1.29E-17 |
| HEMO_64_5402 | 78 | 1.167 | 4.45E-14 | 8.17E-13 |
| HPT_207_121015 | 79 | 1.523 | 5.46E-13 | 8.30E-12 |
| HRG_125_5402 | 80 | 1.205 | 5.79E-23 | 3.08E-21 |
| IC1_238_5412 | 81 | 1.955 | 1.07E-27 | 9.50E-26 |
| IC1_253_5402 | 82 | 1.628 | 6.84E-50 | 1.21E-47 |
| IC1_253_5412 | 83 | 1.847 | 2.14E-58 | 5.70E-56 |
| IC1_253_6503 | 84 | 1.121 | 2.07E-03 | 4.81E-03 |
| IC1_253_6513 | 85 | 1.497 | 3.89E-21 | 1.72E-19 |
| IC1_352_5402 | 86 | 1.571 | 2.25E-61 | 1.20E-58 |
| IGA12_144_3500 | 87 | 1.201 | 3.68E-05 | 1.20E-04 |
| IGG1_297_3410 | 88 | 1.297 | 4.49E-13 | 7.02E-12 |
| IGG1_297_3500 | 89 | 1.372 | 9.07E-03 | 1.81E-02 |
| IGG1_297_3510 | 90 | 1.228 | 3.71E-07 | 1.79E-06 |
| IGG2_297_3500 | 91 | 0.533 | 1.69E-08 | 1.02E-07 |
| IGM_439_9200 | 92 | 2.794 | 4.58E-12 | 5.95E-11 |
| KLKB1_494_6503 | 93 | 1.533 | 1.30E-16 | 3.46E-15 |
| QUANTPEP-A2GL_DLLLPQPDLR | 94 | 1.532 | 8.65E-18 | 6.44E-16 |
| QUANTPEP- | | | | |
| APOA1_DLATVYVDVLK | 95 | 0.697 | 6.52E-19 | 7.77E-17 |
| QUANTPEP-APOM_AFLLTPR | 96 | 0.763 | 1.08E-19 | 1.60E-17 |
| QUANTPEP-B2M_VNHVTLSQPK | 97 | 1.176 | 5.45E-06 | 2.21E-05 |
| QUANTPEP- | 98 | 0.636 | 8.35E-09 | 6.46E-08 |
| FINC_SYTITGLQPGTDYK | | | | |
| QUANTPEP- | 99 | 0.784 | 1.94E-18 | 1.93E-16 |
| TTR_TSESGELHGLTTEEEFVEGIY | | | | |
| K | | | | |
| SHBG_380_5402 | 100 | 1.028 | 5.18E-01 | 6.04E-01 |
| TRFE_432_5401 | 101 | 0.729 | 3.44E-22 | 1.66E-20 |
| VTNC_169_5401 | 102 | 0.71 | 3.25E-19 | 1.15E-17 |

### B. Training a Binary Classification Model

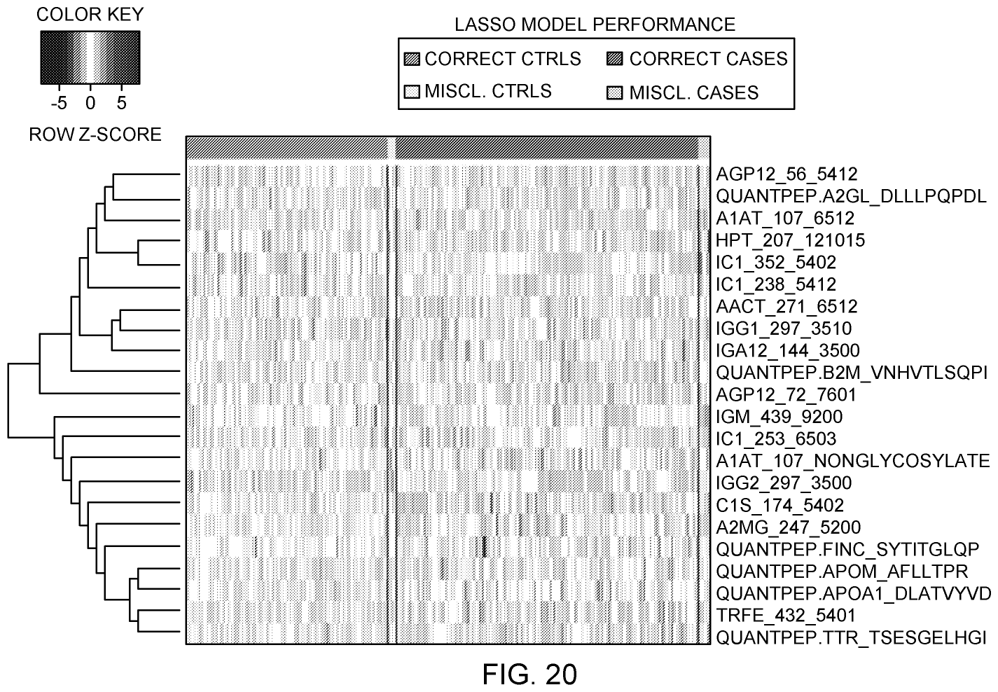

A full panel of biomarkers were included in training a binary classification model for diagnosing pancreatic cancer status. For the various models discussed herein, the total number of subjects was split into 70% training (n=159) and 30% testing (n=67). For the training set, repeated, 10-fold cross-validation was used to select optimal hyperparameters for LASSO, and then these hyperparameters were used on the entire training set develop one predictive logistic regression model. This model was then blindly used to predict pancreatic cancer status in the test set. Overall, 22 markers, 19 markers, or 17 markers were left with non-zero weights after LASSO shrinkage for associated Models 1, 2 and 3, respectively. These 22, 19 and 17 markers are identified in Tables 17A, 17B and 17C above. FIGS. 20-23 are example explanatory illustrations that correspond to Model 1. For example, FIG. 20 is a marker-wise hierarchically-clustered heat map comparing z-score values of biomarker expression levels for retained biomarkers in Model 1 across patent data set, in accordance with one or more embodiments. Columns represent patient samples, grouped by healthy control and pancreatic cancer status, and whether the model correctly or incorrectly classified a specific patient sample.

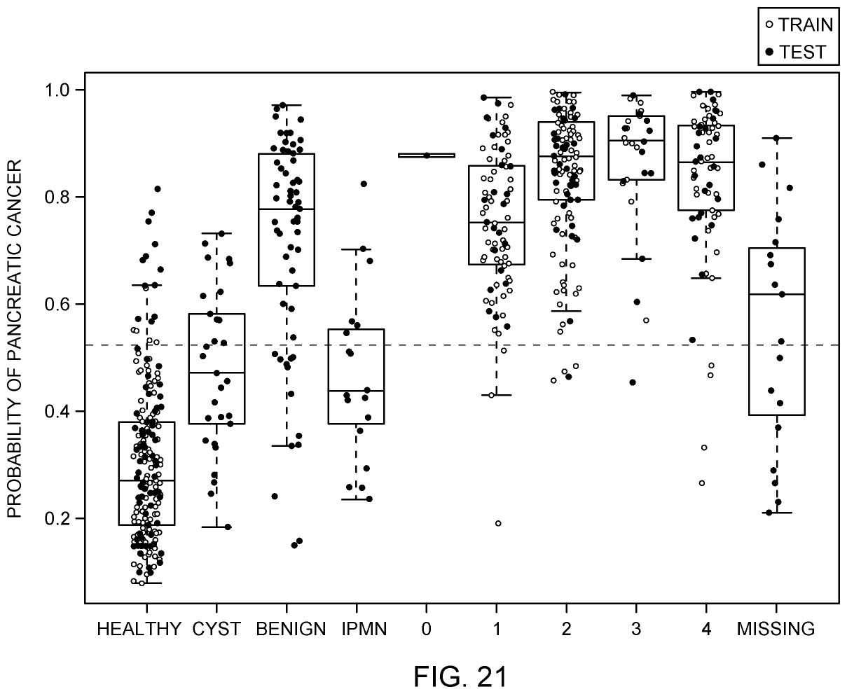

FIG. 21 is a probability dotplot illustrating probabilities of pancreatic cancer across training and test data across various patient sample entities, including pancreatic cancer stage, in accordance with one or more embodiments.

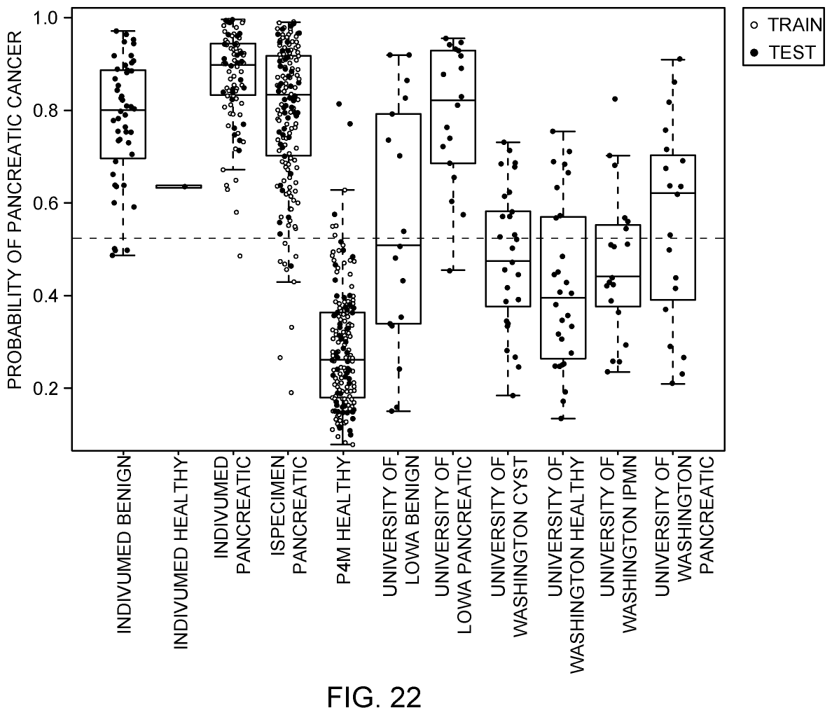

FIG. 22 is a probability dotplot illustrating probabilities of pancreatic cancer across training and test data across various sample sources and entities, in accordance with one or more embodiments.

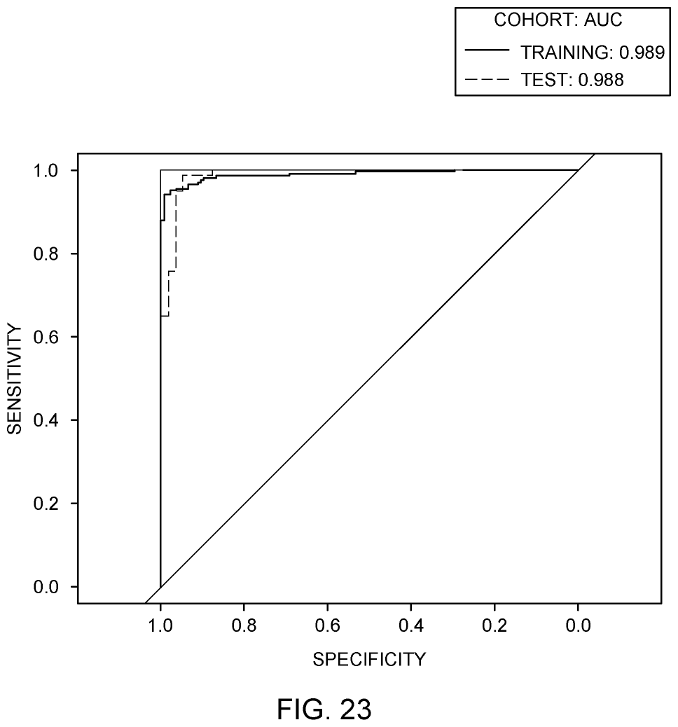

FIG. 23 is an example plot of a receiver operating characteristic (ROC) curve for Model 1 for the training set and testing set in accordance with one or more embodiments. The plot illustrates specificity versus sensitivity. The area under the curve (AUC) for the training set was found to be 0.989 and the AUC for the testing set was found to be 0.988.

### XII. Exemplary Embodiments for Breast Cancer Diagnosis and Treatment

The present disclosure concerns embodiments for systems, methods, and compositions related to identification of breast cancer in an individual. The embodiments concern classifying biological samples, measuring for one or more certain markers from a biological sample, assaying for one or more certain markers from a biological sample, determining the presence of one or more certain markers from a biological sample, and so forth. The embodiments of the disclosure utilize models that accurately either identify that an individual has breast cancer or that has a higher risk for breast cancer over the general population based on the presence of one or more markers in sample(s) from the individual. The individual may or may not be at a higher risk for breast cancer based on family or personal history; age (e.g., 50 or older); having one or more genetic markers associated with a risk for breast cancer (BRCA1 and/or BRCA2); reproductive history (e.g., onset of menses before age 12, starting menopause after age 55, first pregnancy after age 30, not breastfeeding, never having a full-term pregnancy, or a combination thereof); having dense breasts; exposure to radiation therapy; taking hormones; being overweight; having diabetes, workplace exposure to certain chemicals; one or more inherited genetic syndromes; or a combination thereof.

In various embodiments of the disclosure, an individual is in need of identifying whether or not they have breast cancer or a risk thereof. The individual may be subjected to measuring or testing for one or more markers encompassed herein as a matter of routine health maintenance or because of a specific concern, for example, such as the presence of one or more risk factors and/or one or more symptoms of breast cancer. The individual may be in need of such identification based on any one of the risk factors noted above, or the individual may be in need of such identification based on having one or more symptoms of breast cancer, such as having a new lump in the breast or armpit; thickening or swelling of part of the breast; irritation or dimpling of breast skin; redness or flaky skin in the nipple area of the breast; pulling in of the nipple or pain in the nipple area; or a combination thereof. In some cases, the analysis of the sample of the individual as described herein is the sole test utilized for identifying breast cancer, whereas in other cases a medical provider may utilize one or more other tests, such as mammogram; ultrasound; magnetic resonance imaging; CT scan; biopsy; a combination thereof, and so forth. In particular embodiments, measuring for one or more peptide structure markers as in Table 9 are utilized alone or in conjunction with one or more of these tests.

The systems, methods, and compositions encompassed herein are sufficiently specific to utilize markers that distinguish between control and breast cancer or between control and early stage breast cancer. In some embodiments, the markers are accurate regardless of the status of one or more characteristics of the individual: biological sex, sample source, sample collection, smoker status, or age, as examples.

In some embodiments, the individual is suspected of having breast cancer or is at risk for breast cancer and is in need of diagnosis thereof in addition to identification whether it is early stage breast cancer. In various embodiments, the individual is known to have breast cancer and is in need of determining whether it is early stage breast cancer, such as to determine a treatment regimen for the cancer. In specific embodiments, the same test that identifies whether an individual has breast cancer determines whether the breast cancer is early stage.

In various embodiments, the sample for analysis for breast cancer identification is a fluid from the individual, such as peripheral blood, serum, plasma, and/or nipple aspirate from the individual. The present disclosure provides for measuring for one or more circulating glycoproteins, glycopeptides, or non-glycosylated peptides in blood, serum, or plasma to diagnose or identify the presence of breast cancer and/or to identify early stage breast cancer in an individual. In various embodiments, the sample is measured for 1, 2, 3, 4, 5, 6, 7, 8, 9, 10, 11, 12, 13, 14, 15, 16, 17, or all 18 of the peptides of Table 9. In various embodiments, the sample is measured for 1, 2, 3, 4, 5, 6, or all 7 of the peptides of Table 10A. In various embodiments, the sample is measured for 1, 2, 3, 4, 5, 6, 7, 8 or all 8 of the peptides of Table 10B.

Embodiments of the disclosure include methods of classifying samples, including peripheral blood, serum, or plasma samples, from an individual suspected of having, known to have, or at risk for having breast cancer by measuring from the sample for one or more glycopeptides and/or non-glycosylated peptides encompassed herein. The methods encompass whether or not breast cancer is identified in the individual. In some cases, the measuring identifies the individual as not having breast cancer or as having breast cancer. In various embodiments, in cases wherein the individual has one or more glycopeptides and/or non-glycosylated peptides of Table 9, or certain levels thereof compared to control or healthy individuals, the individual may be determined to have breast cancer. In various embodiments, in cases wherein the individual lacks the glycopeptides and/or non-glycosylated peptides of Table 9, or has certain levels thereof compared to control or healthy individuals, the individual may be determined not to have breast cancer. The measuring may identify the individual as having a particular stage of breast cancer, including at least early stage. In specific cases, the measuring comprises successive or concomitant steps of identifying that the individual has breast cancer and whether the individual has early stage breast cancer.

In various embodiments, an individual at risk for having breast cancer is subjected to methods of the disclosure to identify, or not, the presence of breast cancer. Such methods also measure for one or more glycopeptides and/or non-glycosylated peptides encompassed herein. In various embodiments, in cases wherein the individual has one or more glycopeptides and/or non-glycosylated peptides of Table 9, the individual may be determined to have breast cancer. In various embodiments, in cases wherein the individual lacks the glycopeptides and/or non-glycosylated peptides of Table 9, the individual may be determined not to have breast cancer and is not treated for breast cancer. The individual may be of any kind, although in specific cases individual at risk for having breast cancer has a family history or one or more other risk factors.

Embodiments of the disclosure include methods of predicting that an individual will have breast cancer, including early stage breast cancer, or identifying early stage breast cancer in an individual, by measuring for one or more glycopeptides or non-glycosylated peptides from Table 9 in one or more samples from the individual. The individual may be known to have breast cancer or may be suspected of having breast cancer In various embodiments, the sample is measured for 1, 2, 3, 4, 5, 6, 7, 8, 9, 10, 11, 12, 13, 14, 15, 16, 17, or all 18 of the peptides of Table 9. In various embodiments, the sample is measured for 1, 2, 3, 4, 5, 6, or all 7 of the peptides of Table 10A. In various embodiments, the sample is measured for 1, 2, 3, 4, 5, 6, 7, or all 8 of the peptides of Table 10B.

In embodiments wherein the measuring identifies the individual as having breast cancer, the individual may be recommended to take action to treat the breast cancer, such as with at least one of radiation therapy, chemotherapy or drug therapy (Abemaciclib; Ado-Trastuzumab Emtansine; Everolimus; Alpelisib; Anastrozole; Pamidronate Disodium; Anastrozole; Exemestane; Capecitabine; Cyclophosphamide; Docetaxel; Doxorubicin Hydrochloride; Epirubicin Hydrochloride; Fam-Trastuzumab Deruxtecan-nxki; Epirubicin Hydrochloride; Eribulin Mesylate; 5-Fluoro uracil; Fam-Trastuzumab Deruxtecan-nxki; Toremifene; Fulvestrant; Letrozole; Fluorouracil Injection; Fulvestrant; Gemcitabine Hydrochloride; Goserelin Acetate; Eribulin Mesylate; Trastuzumab; Palbociclib; Gemcitabine Hydrochloride; Ixabepilone; Ixabepilone; Ado-Trastuzumab Emtansine; Pembrolizumab; Ribociclib; Lapatinib Ditosylate; Letrozole; Olaparib; Margetuximab-cmkb; Megestrol Acetate; Methotrexate Sodium; Neratinib Maleate; Neratinib Maleate; Olaparib; Paclitaxel; Palbociclib; Pamidronate Disodium; Pembrolizumab; Pertuzumab; Alpelisib; Ribociclib; Sacituzumab Govitecan-hziy; Tamoxifen Citrate; Talazoparib Tosylate; Talazoparib Tosylate; Tamoxifen Citrate; Docetaxel; Atezolizumab; Thiotepa; Thiotepa; Toremifene; Trastuzumab; Trastuzumab and Hyaluronidase-oysk; Methotrexate Sodium; Sacituzumab Govitecan-hziy; Tucatinib; Tucatinib; Lapatinib Ditosylate; Abemaciclib; Vinblastine Sulfate; Capecitabine; Goserelin Acetate; or a combination thereof.), chemoradiotherapy, surgery, hormone therapy and/or a targeted drug therapy, as examples.

Embodiments of the disclosure include methods of treating breast cancer in a subject, the method comprising: receiving a biological sample from the subject; determining a quantity of at least 1 peptide structure identified in Table 9 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has breast cancer; and administering a therapeutically effective amount of the treatment for breast cancer. The treatment may be of any kind, including at least one or more of radiation therapy, chemotherapy, chemoradiotherapy, surgery, or a targeted drug therapy. In specific embodiments, the method further comprises preparing the biological sample to form a prepared sample comprising a set of peptide structures; and inputting the prepared sample into the MRM-MS system using a liquid chromatography system. The method may also be further defined as determining a quantity of at least 1 peptide structure identified in Table 9 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has BC; and administering a therapeutically effective amount of the treatment for BC.

Embodiments of the disclosure include methods of treating breast cancer (BC) in a subject, the method comprising: receiving a biological sample from the subject; determining a quantity of at least 1 peptide structure identified in Table 10A or 10B in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the patient has breast cancer; and administering a therapeutically effective amount of the treatment for breast cancer, such as at least one of radiation therapy, chemotherapy, chemoradiotherapy, surgery, or a targeted drug therapy. In various embodiments, the method may further comprise preparing the biological sample to form a prepared sample comprising a set of peptide structures; and inputting the prepared sample into the MRM-MS system using a liquid chromatography system. In specific embodiments, the method may be further defined as determining a quantity of at least 1 peptide structure identified in Table 10A or 10B in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has breast cancer; and administering a therapeutically effective amount of the treatment for breast cancer.

Certain embodiments of the disclosure encompass methods of designing a treatment for a subject diagnosed with a breast cancer state, the method comprising: designing a therapeutic regimen for treating the subject in response to measuring that a biological sample obtained from the subject evidences the state using part or all of any method encompassed herein, including identifying one or more peptide structures of Table 9, 10A, or 10B. Various embodiments include methods of planning a treatment for a subject diagnosed with a breast cancer (BC) state, the method comprising: generating a treatment plan for treating the subject in response to measuring that a biological sample obtained from the subject evidences the state using part or all of any method encompassed herein, including identifying one or more peptide structures of Table 9, 10A, or 10B.

Embodiments of the disclosure include methods of treating a subject diagnosed with a breast cancer state, the method comprising: administering to the subject a therapeutically effective amount of one or more therapeutics or treatments to treat the subject based on measuring that a biological sample obtained from the subject evidences the state using part or all of any method encompassed herein, including that identifies one or more peptide structures of Table 9, 10A, or 10B.

In various embodiments, methods of treating a subject diagnosed with a breast cancer state are encompassed herein, the method comprising: selecting a therapeutic or treatment to treat the subject based on determining that the subject is responsive to the therapeutic using any method encompassed herein, including that identifies one or more peptide structures of Table 9, 10A, or 10B.

In various embodiments, methods are included for classifying a sample from an individual suspected of having, known to have, or at risk for breast cancer, comprising the step of measuring from the sample for one or more glycopeptides and/or non-glycosylated peptides in Table 9. In specific embodiments, the measuring identifies the individual as not having breast cancer or as having breast cancer. The measuring may identify the individual as having early stage BC, in specific embodiments, and the detection of early stage malignancy is useful such that a treatment path may be determined as soon as possible. In certain embodiments, the measuring comprises successive or concomitant steps of identifying that the individual has breast cancer and that the individual has early stage breast cancer. The individual may or may not be at risk for breast cancer. In specific cases, when the measuring identifies the individual as having breast cancer, the individual is administered an effective amount of at least one of radiation therapy, chemotherapy, chemoradiotherapy, surgery, or a targeted drug therapy. In various embodiments, the sample is measured for 2, 3, 4, 5, 6, 7, 8, 9, 10, 11, 12, 13, 14, 15, 16, 17, or all 18 of the glycopeptides and/or non-glycosylated peptides of Table 9.

Embodiments of the disclosure include methods of diagnosing breast cancer in an individual, comprising the step of identifying 1, 2, 3, 4, 5, 6, 7, 8, 9, 10, 11, 12, 13, 14, 15, 16, 17, or all 18 of the peptide structures identified in Table 9 from a sample from the individual. Embodiments of the disclosure include methods of diagnosing breast cancer in an individual, comprising the step of identifying 1, 2, 3, 4, 5, 6, or all 7 of the peptide structures identified in Table 10A from a sample from the individual. Embodiments of the disclosure include methods of diagnosing breast cancer in an individual, comprising the step of identifying 1, 2, 3, 4, 5, 6, 7, or all 8 of the peptide structures identified in Table 10B from a sample from the individual.

### XIII

### Exemplary Embodiments for Pancreatic Cancer Diagnosis and Treatment

The present disclosure concerns embodiments for systems, methods, and compositions related to identification of pancreatic cancer in an individual. The embodiments concern classifying biological samples, measuring for one or more certain markers from a biological sample, assaying for one or more certain markers from a biological sample, determining the presence of one or more certain markers from a biological sample, and so forth. The embodiments of the disclosure utilize models that accurately either identify that an individual has pancreatic cancer or that has a higher risk for pancreatic cancer over the general population based on the presence of one or more markers in sample(s) from the individual. The individual may or may not be at a higher risk for pancreatic cancer based on family history, one or more genetic markers associated with a risk for pancreatic cancer, being overweight, having diabetes, having chronic pancreatitis, workplace exposure to certain chemicals, advanced age, one or more inherited genetic syndromes, a diet rich in red and/or processed meats, or a combination thereof.

In various embodiments of the disclosure, an individual is in need of identifying whether or not they have pancreatic cancer or a risk thereof. The individual may be subjected to measuring or testing for one or more markers encompassed herein as a matter of routine health maintenance or because of a specific concern, for example, such as the presence of one or more risk factors and/or one or more symptoms of pancreatic cancer. The individual may be in need of such identification based on any one of the risk factors noted above, or the individual may be in need of such identification based on having one or more symptoms of pancreatic cancer, such as abdominal pain, including that radiates to the back; loss of appetite or unintended weight loss; jaundice; light-colored stools; dark-colored urine; itchy skin; new diagnosis of diabetes or existing diabetes that's becoming more difficult to control; blood clots; or a combination thereof. In some cases, the analysis of the sample of the individual is the sole test utilized for identifying pancreatic cancer, whereas in other cases a medical provider may utilize one or more other tests, such as ultrasound, magnetic resonance imaging, CT scan, Endoscopic retrograde cholangiopancreatography, Magnetic resonance cholangiopancreatography, Percutaneous transhepatic cholangiography, Positron emission tomography (PET) scan, angiography, biopsy, blood tests (Such as measuring for CA 19-9 and/or carcinoembryonic antigen (CEA), a combination thereof, and so forth. In particular embodiments, measuring for one or more peptide structure markers as in Table 16 are utilized alone or in conjunction with one or more of these tests.

The systems, methods, and compositions encompassed herein are sufficiently specific to utilize markers that distinguish between control and pancreatic cancer, including in various embodiments markers that themselves are not associated with inflammation, such as inflammation associated with pancreatic cancer. In some embodiments, the markers are accurate regardless of the status of one or more characteristics of the individual: biological sex, sample source, sample collection, smoker status, or age.

In some embodiments, the individual is suspected or having pancreatic cancer or is at risk for pancreatic cancer and is in need of diagnosis thereof in addition to identification whether it is early stage pancreatic cancer. In various embodiments, the individual is known to have pancreatic cancer and is in need of determining whether it is early stage pancreatic cancer, such as to determine a treatment regimen for the cancer. In specific embodiments, the same test that identifies whether an individual has pancreatic cancer determines whether the pancreatic cancer is early stage.

In various embodiments, the sample for analysis for pancreatic cancer identification is a fluid from the individual, such as peripheral blood, serum, or plasma from the individual. The present disclosure provides for measuring for one or more circulating glycoproteins, glycopeptides, or non-glycosylated peptides in blood, serum, or plasma to diagnose or identify the presence of pancreatic cancer and/or to identify early stage pancreatic cancer in an individual. In various embodiments, the sample is measured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or all 55 of the peptides of Table 16. In various embodiments, the sample is measured for 1, 2, 3, 4, 5, 6, 7, 8, 9, 10, 11, 12, 13, 14, 15, 16, 17, 18, 19, 20, 21, or all 22 of the peptides of Table 17A. In various embodiments, the sample is measured for 1, 2, 3, 4, 5, 6, 7, 8, 9, 10, 11, 12, 13, 14, 15, 16, 17, 18, or all 19 of the peptides of Table 17B. In various embodiments, the sample is measured for 1, 2, 3, 4, 5, 6, 7, 8, 9, 10, 11, 12, 13, 14, 15, 16, or all 17 of the peptides of Table 17C.

Embodiments of the disclosure include methods of classifying samples, including peripheral blood, serum, or plasma samples, from an individual suspected of having, known to have, or at risk for having pancreatic cancer by measuring from the sample for one or more glycopeptides and/or non-glycosylated peptides encompassed herein. The methods encompass whether or not pancreatic cancer is identified in the individual. In some cases, the measuring identifies the individual as not having pancreatic cancer or as having pancreatic cancer. In various embodiments, in cases wherein the individual has one or more glycopeptides and/or non-glycosylated peptides of Table 16, or certain levels thereof compared to control or healthy individuals, the individual may be determined to have pancreatic cancer. In various embodiments, in cases wherein the individual lacks the glycopeptides and/or non-glycosylated peptides of Table 16, or has certain levels thereof compared to control or healthy individuals, the individual may be determined not to have pancreatic cancer. The measuring may identify the individual as having a particular stage of pancreatic cancer, including at least early stage. In specific cases, the measuring comprises successive or concomitant steps of identifying that the individual has pancreatic cancer and whether the individual has early stage pancreatic cancer.

In various embodiments, an individual at risk for having pancreatic cancer is subjected to methods of the disclosure to identify, or not, the presence of pancreatic cancer. Such methods also measure for one or more glycopeptides and/or non-glycosylated peptides encompassed herein. In various embodiments, in cases wherein the individual has one or more glycopeptides and/or non-glycosylated peptides of Table 16, the individual may be determined to have pancreatic cancer. In various embodiments, in cases wherein the individual lacks the glycopeptides and/or non-glycosylated peptides of Table 16, the individual may be determined not to have pancreatic cancer and is not treated for pancreatic cancer. The individual may be of any kind, although in specific cases individual at risk for having pancreatic cancer has a family history or one or more other risk factors.

Embodiments of the disclosure include methods of predicting that an individual will have pancreatic cancer, including early stage pancreatic cancer, or identifying early stage pancreatic cancer in an individual, by measuring for one or more glycopeptides or non-glycosylated peptides from Table 16 in one or more samples from the individual. The individual may be known to have pancreatic cancer or may be suspected of having pancreatic cancer In various embodiments, the sample is measured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or all 55 of the peptides of Table 16. In various embodiments, the sample is measured for 1, 2, 3, 4, 5, 6, 7, 8, 9, 10, 11, 12, 13, 14, 15, 16, 17, 18, 19, 20, 21, or all 22 of the peptides of Table 17A. In various embodiments, the sample is measured for 1, 2, 3, 4, 5, 6, 7, 8, 9, 10, 11, 12, 13, 14, 15, 16, 17, 18, or all 19 of the peptides of Table 17B. In various embodiments, the sample is measured for 1, 2, 3, 4, 5, 6, 7, 8, 9, 10, 11, 12, 13, 14, 15, 16, or all 17 of the peptides of Table 17C.

In embodiments wherein the measuring identifies the individual as having pancreatic cancer, the individual may be recommended to take action to treat the pancreatic cancer, such as with at least one of radiation therapy, chemotherapy (Gemcitabine; 5-fluorouracil (5-FU); Oxaliplatin; Albumin-bound paclitaxel; Capecitabine; Cisplatin; Irinotecan; Paclitaxel; and/or Docetaxel), chemoradiotherapy, surgery, and/or a targeted drug therapy, as examples.

Embodiments of the disclosure include methods of treating pancreatic cancer in a subject, the method comprising: receiving a biological sample from the subject; determining a quantity of at least 1 peptide structure identified in Table 16 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has pancreatic cancer; and administering a therapeutically effective amount of the treatment for pancreatic cancer. The treatment may be of any kind, including at least one or more of radiation therapy, chemotherapy, chemoradiotherapy, surgery, or a targeted drug therapy. In specific embodiments, the method further comprises preparing the biological sample to form a prepared sample comprising a set of peptide structures; and inputting the prepared sample into the MRM-MS system using a liquid chromatography system. The method may also be further defined as determining a quantity of at least 1 peptide structure identified in Table 16 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has PC; and administering a therapeutically effective amount of the treatment for PC.

Embodiments of the disclosure include methods of treating pancreatic cancer (PC) in a subject, the method comprising: receiving a biological sample from the subject; determining a quantity of at least 1 peptide structure identified in Table 17A, 17B, or 17C in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the patient has pancreatic cancer; and administering a therapeutically effective amount of the treatment for pancreatic cancer, such as at least one of radiation therapy, chemotherapy, chemoradiotherapy, surgery, or a targeted drug therapy. In various embodiments, the method may further comprise preparing the biological sample to form a prepared sample comprising a set of peptide structures; and inputting the prepared sample into the MRM-MS system using a liquid chromatography system. In specific embodiments, the method may be further defined as determining a quantity of at least 1 peptide structure identified in Table 17A, 17B, or 17C in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has pancreatic cancer; and administering a therapeutically effective amount of the treatment for pancreatic cancer.

Certain embodiments of the disclosure encompass methods of designing a treatment for a subject diagnosed with a pancreatic cancer state, the method comprising: designing a therapeutic regimen for treating the subject in response to measuring that a biological sample obtained from the subject evidences the state using part or all of any method encompassed herein, including identifying one or more peptide structures of Table 16, 17A, 17B, or 17C. Various embodiments include methods of planning a treatment for a subject diagnosed with a pancreatic cancer (PC) state, the method comprising: generating a treatment plan for treating the subject in response to measuring that a biological sample obtained from the subject evidences the state using part or all of any method encompassed herein, including identifying one or more peptide structures of Table 16, 17A, 17B, or 17C.

Embodiments of the disclosure include methods of treating a subject diagnosed with a pancreatic cancer state, the method comprising: administering to the subject a therapeutically effective amount of one or more therapeutics or treatments to treat the subject based on measuring that a biological sample obtained from the subject evidences the state using part or all of any method encompassed herein, including that identifies one or more peptide structures of Table 16, 17A, 17B, or 17C.

In various embodiments, methods of treating a subject diagnosed with a pancreatic cancer state are encompassed herein, the method comprising: selecting a therapeutic or treatment to treat the subject based on determining that the subject is responsive to the therapeutic using any method encompassed herein, including that identifies one or more peptide structures of Table 16, 17A, 17B, or 17C.

In various embodiments, methods are included for classifying a sample from an individual suspected of having, known to have, or at risk for pancreatic cancer, comprising the step of measuring from the sample for one or more glycopeptides and/or non-glycosylated peptides in Table 16. In specific embodiments, the measuring identifies the individual as not having pancreatic cancer or as having pancreatic cancer. The measuring may identify the individual as having early stage PC, in specific embodiments, and the detection of early stage malignancy is useful such that a treatment path may be determined as soon as possible. In certain embodiments, the measuring comprises successive or concomitant steps of identifying that the individual has pancreatic cancer and that the individual has early stage pancreatic cancer. The individual may or may not be at risk for pancreatic cancer. In specific cases, when the measuring identifies the individual as having pancreatic cancer, the individual is administered an effective amount of at least one of radiation therapy, chemotherapy, chemoradiotherapy, surgery, or a targeted drug therapy. In various embodiments, the sample is measured for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or all of the glycopeptides and/or non-glycosylated peptides of Table 16.

Embodiments of the disclosure include methods of diagnosing pancreatic cancer in an individual, comprising the step of identifying 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or all 55 of the peptide structures identified in Table 16 from a sample from the individual. Embodiments of the disclosure include methods of diagnosing pancreatic cancer in an individual, comprising the step of identifying 1, 2, 3, 4, 5, 6, 7, 8, 9, 10, 11, 12, 13, 14, 15, 16, 17, 18, 19, 20, 21, or all 22 of the peptide structures identified in Table 17A from a sample from the individual. Embodiments of the disclosure include methods of diagnosing pancreatic cancer in an individual, comprising the step of identifying 1, 2, 3, 4, 5, 6, 7, 8, 9, 10, 11, 12, 13, 14, 15, 16, 17, 18, or all 19 of the peptide structures identified in Table 17B from a sample from the individual. Embodiments of the disclosure include methods of diagnosing pancreatic cancer in an individual, comprising the step of identifying 1, 2, 3, 4, 5, 6, 7, 8, 9, 10, 11, 12, 13, 14, 15, 16, or all 17 of the peptide structures identified in Table 17C from a sample from the individual.

### XIV

### Exemplary Methodologies Relating to Quality Control of Samples Based on Peptide Structure Data Analysis

### XIV. A. Exemplary Methodology—Based on Table 23

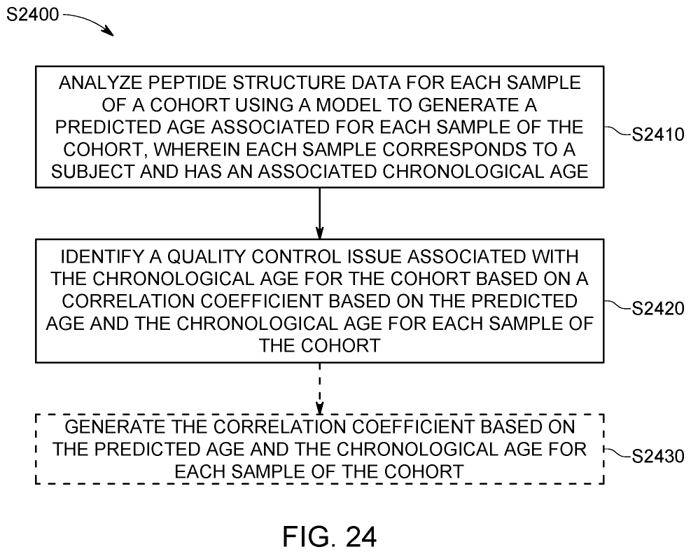

FIG. 24 is a flowchart of a method S2400 for quality control (QC) of samples, in accordance with various embodiments. Method S2400 may be implemented using, for example, at least a portion of workflow 100 as described in FIGS. 1, 2A, and 2B and/or analysis system 300 as described in FIG. 3. Method S2400 may be used to generate a final output that includes at least an identification of control issue associated with a batch or a cohort of samples.

As illustrated in FIG. 24, method S2400 includes, at step S2410, analyzing peptide structure data for each sample of a cohort using a model to generate a predicted age associated for each sample of the cohort, wherein each sample corresponds to a subject and has an associated chronological age. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 23, with the peptide sequence being one of SEQ ID NOS: 163-174 in Table 23 below.

In one or more embodiments, the model may be implemented using a binary classification model (e.g., a linear regression model). In some examples, the regression model may be, for example, penalized multivariable regression model. In various embodiments, the quantification data may be computed using a weight coefficient associated with each peptide structure, the weight coefficient of a corresponding peptide structure of the peptide structures may indicate the relative significance of the corresponding peptide structure to the disease indicator.

In some embodiments, the model may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature-relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

As illustrated in FIG. 24, method S2400 further includes, at step S2420, identifying a quality control issue associated with the chronological age for the cohort based on a correlation coefficient based on the predicted age and the chronological age for each sample of the cohort. In various embodiments, the identifying the quality control issue associated with the chronological age for the cohort is based on the correlation coefficient. In various embodiments, the correlation coefficient does not fall within a predetermined range of values. In various embodiments, the predetermined range of values ranges from about 0 to about 0.2. In various embodiments, the quality control issue includes an error of mislabeled samples or an error from sample preparation. In various embodiments, the quality control issue includes a systemic measurement or an instrument error.

In various embodiments, the peptide structure data can include a set of age-associated glycosylation biomarkers, and a set of corresponding signals associated with each of the age-associated glycosylation biomarkers. In various embodiments, the set of corresponding signals is proportional to an amount of each of the age-associated glycosylation biomarkers in the sample. In various embodiments, the model can be based on the set of age-associated glycosylation biomarkers and the set of corresponding signals associated with each of the age-associated glycosylation biomarkers. In various embodiments, the set of age-associated glycosylation biomarkers comprises at least one of the age-associated glycosylation biomarkers listed in Table 23. Table 23 below lists a first group of peptide structures associated with age or a range of ages.

In various embodiments, method S2400 may optionally include, at step S2430, generating the correlation coefficient based on the predicted age and the chronological age for each sample of the cohort. In various embodiments, the correlation coefficient can be a Pearson correlation coefficient where the predicted age is a continuous variable and the chronological age is another continuous variable. In various embodiments, the Pearson correlation coefficient (r_{xy}) can be determined via an equation:

$${r}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$

$$where n = a number of the samples in the cohort ,$$

$$i = an index number for each of the samples ,$$

$${x}_{i} = a chronological age for sample i ,$$

$$\overline{x} = a mean chronological age ,$$

$${y}_{i} = a predicted age for sample i , and$$

$$\overline{y} = a mean predicted age .$$

In various embodiments, the model can include multiplying the corresponding signal associated with age-associated glycosylation biomarkers and a respective coefficient for each sample of the cohort to form a plurality of products. In various embodiments, the model can also include summing together the plurality of products to form a summation, and then adding the summation and the intercept to form an output value, wherein the output value is proportional to the predicted age for the sample. In various embodiments, the model can include an equation:

$$O V = {\sum }_{i = 1}^{i = 20} [ ( {Signal}_{SEQ ID No : i} ) \times ( {Coefficient}_{SEQ ID No : i} ) ] + Intercept$$

$$where O V = an output value ,$$

$$i = an index number for each of the age - associated glycosylation biomarkers ,$$

$${Signal}_{SEQ ID No : i} = a corresponding signal associated with the age - associated glycosylation biomarker i , and$$

$${Coefficient}_{SEQ ID No : i} = a coefficient associated with the age - associated glycosylation biomarker i .$$

In various embodiments, each sample of the cohort has a disease condition, where the disease condition selected from the group consisting of non-small cell lung cancer, breast cancer, pancreatic cancer, colorectal cancer, and nonalcoholic steatohepatitis (NASH). In various embodiments, each sample of the cohort has either a disease condition or a healthy condition, where the disease condition selected from the group consisting of non-small cell lung cancer, breast cancer, pancreatic cancer, colorectal cancer, and nonalcoholic steatohepatitis (NASH). In various embodiments, each sample of the cohort has either a single disease condition or a healthy condition where the disease conditions include non-small cell lung cancer, breast cancer, pancreatic cancer, colorectal cancer, and nonalcoholic steatohepatitis (NASH). In various embodiments, at least one of the age-associated glycosylation biomarkers includes a glycopeptide structure defined by a peptide sequence and a glycan structure linked to the peptide sequence at a linking site of the peptide sequence, as identified in Table 23, with the peptide sequence being one of SEQ ID NOS: 163-174 as defined in Table 25A and 25B.

In various embodiments, method S2400 can optionally include receiving the peptide structure data for each sample of the cohort from a mass spectrometer. In various embodiments, the peptide structure data include at least one of a raw abundance, an adjusted raw abundance, a peptide concentration, a glycopeptide concentration, or a normalized concentration. In various embodiments, the peptide structure data comprise normalized concentration data, wherein the normalized concentration data is a function of at least one of peptide abundance data, corresponding internal standard abundance data, a spike-in concentration value, and a dilution factor. In various embodiments, the peptide structure data are generated using multiple reaction monitoring mass spectrometry (MR n-MS).

In various embodiments, method S2400 can optionally include creating a sample from the biological sample, and preparing the sample using reduction, alkylation, and enzymatic digestion to form a prepared sample that includes a set of peptide structures. In various embodiments, method S2400 can optionally include generating the peptide structure data from the prepared sample using multiple reaction monitoring mass spectrometry (MR-MS).

In various embodiments, the set of age-associated glycosylation biomarkers can include at least three of the age-associated glycosylation biomarkers listed in Table 23. In various embodiments, the set of age-associated glycosylation biomarkers can include at least five of the age-associated glycosylation biomarkers listed in Table 23.

Table 23 below lists a group of peptide structures associated with age or a range of ages. One or more features (e.g., relative abundance, concentration, site occupancy) of these peptide structures may be used in a supervised machine learning model described elsewhere herein to generate an age-related indicator.

TABLE 23
Peptide Structures Associated with Age or a Range of Ages

| | | | | | Linking | Linking | |
|---|---|---|---|---|---|---|---|
| | | | | | Site | Site | |
| | | | | | Position | Position | |
| | | | (Protein) | (Peptide) | within | within | Glycan |
| | PS-ID | | SEQ ID | SEQ ID | Protein | Peptide | Structure |
| PS-NAME | NO. | Protein Name | NO. | NO. | Sequence | Sequence | GL NO. |
| IC1 (352) - 5402 | 103 | Plasma protease C1 inhibitor | 153 | 163 | 352 | 9 | 5402 |
| IC1 (253) - 6513 | 104 | Plasma protease C1 inhibitor | 153 | 164 | 253 | 4 | 6513 |
| IGG1 (297) - 5410 | 105 | Immunoglobulin heavy constant gamma 1 | 154 | 165 | 297 | 5 | 5410 |
| IGA12 (144MC) - 4500 | 106 | Immunoglobulin heavy constant alpha 1 or 2 | 155 or 160 | 166 | 144 | 13 | 4500 |
| ZA2G (128) - 5402 | 107 | Zinc-alpha-2-glycoprotein | 156 | 167 | 128 | 8 | 5402 |
| IGG1 (297) - 3510 | 108 | Immunoglobulin heavy constant gamma 1 | 154 | 165 | 297 | 5 | 3510 |
| IGG2 (297) - 5510 | 109 | Immunoglobulin heavy constant gamma 2 | 157 | 168 | 297 | 5 | 5510 |
| IGG1 (297) - 5411 | 110 | Immunoglobulin heavy constant gamma 1 | 154 | 165 | 297 | 5 | 5411 |
| AGP12 (72MC) - 6503 | 111 | Alpha-1-acid glycoprotein 1&2 | 158 or 162 | 169 | 72 | 15 | 6503 |
| IGG1 (297) - 4510 | 112 | Immunoglobulin heavy constant gamma 1 | 154 | 165 | 297 | 5 | 4510 |
| IC1 (253) - 6503 | 113 | Plasma protease C1 inhibitor | 153 | 164 | 253 | 4 | 6503 |
| TRFE (630) - 5402 | 114 | Serotransferrin | 159 | 170 | 630 | 9 | 5402 |
| IGA2 (205) - 5511 | 115 | Immunoglobulin heavy constant alpha 2 | 160 | 171 | 205 | 6 | 5511 |
| AGP12 (72MC) - 7613 | 116 | Alpha-1-acid glycoprotein 1 or 2 | 158 or 162 | 169 | 72 | 15 | 7613 |
| AGP12 (72) - 7601 | 117 | Alpha-1-acid glycoprotein 1 or 2 | 158 or 162 | 172 | 72 | 15 | 7601 |
| IGA12 (144) - 5402 | 118 | Immunoglobulin heavy constant alpha 1 or 2 | 155 or 160 | 173 | 144 | 18 | 5402 |
| IGG1 (297) - 3410 | 119 | Immunoglobulin heavy constant gamma 1 | 154 | 165 | 297 | 5 | 3410 |
| IGM (209) - 5411_Z3 | 120 | Immunoglobulin heavy constant mu | 161 | 174 | 209 | 7 | 5411 |
| IGG1 (297) - 5412 | 121 | Immunoglobulin heavy constant gamma 1 | 154 | 165 | 297 | 5 | 5412 |
| IGG1 (297) - 3500 | 122 | Immunoglobulin heavy constant gamma 1 | 154 | 165 | 297 | 5 | 3500 |

Table 23 includes the Peptide Structure Identification Number (PS-ID NO.) that is a reference number for a particular peptide or glycopeptide. The Peptide Structure Name (PS-Name, e.g., IC1 (352)-5402), which is a reference code for the protein name (e.g., ICI), followed by the glycan linking site position in the protein (e.g., the number 352 that is within a parenthesis and represents a sequential amino acid position in protein IC1), and followed by the glycan structure GL number (e.g., the number 5402 that is preceded by a hyphen and represents a glycan composition Hex(5)HexNAc(4)Fuc(0)NeuAc(2)). The Protein Sequence ID No of Table 23 corresponds to the corresponding protein name, Unitprot ID, and amino acid sequence of Table 26. The Peptide Sequence ID No of Table 23 corresponds to the corresponding peptide sequence of Table 25A. The term Linking Site Pos. within Protein Sequence is a number that refers to the sequential position of an amino acid of the corresponding protein in which a glycan is attached. For the Glycan Linking Site Pos. within Protein Sequence, the amino acid position of the peptide sequence is defined by the sequentially numbered order of amino acids based on the Uniprot ID of the corresponding protein for the peptide sequence. The term Linking Site Pos. within Peptide Sequence is a number that refers to the sequential position of an amino acid of the corresponding peptide in which a glycan is attached. For the Glycan Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Table 27.

In some embodiments, the term AGP12 represents that the glycopeptide is a fragment of either AGP1 or AGP2. In some embodiments, the term IGA12 represents that the glycopeptide is a fragment of either IGA1 or IGA2.

In some instances of the Peptide Structure (PS) NAME, subsequent to the prefix, there is a number noted with the notation MC that indicates that there was a missed cleavage at position in the peptide sequence as noted by the number. In some instances of the Peptide Structure (PS) NAME, there is a suffix NH₃LOSS to indicate a loss of a NH₃ group.

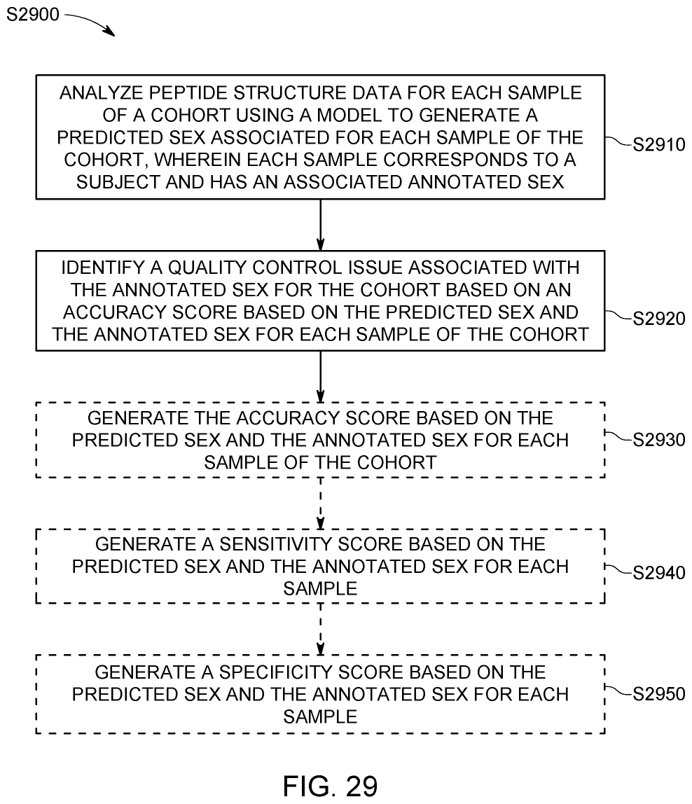

As illustrated in FIG. 29, method S2900 further includes, at step S2920, identifying a quality control issue associated with the annotated sex for the cohort based on an accuracy score based on the predicted sex and the annotated sex for each sample of the cohort. In various embodiments, the identifying the quality control issue associated with the annotated sex for the cohort is based on the accuracy score. In various embodiments, the quality control issue includes an error of mislabeled samples or an error from sample preparation. In various embodiments, the quality control issue includes a systemic measurement or an instrument error.

In various embodiments, the peptide structure data can include a set of sex-associated glycosylation biomarkers, and a set of corresponding signals associated with each of the sex-associated glycosylation biomarkers. In various embodiments, the set of corresponding signals is proportional to an amount of each of the sex-associated glycosylation biomarkers in the sample. In various embodiments, the model can be based on the set of sex-associated glycosylation biomarkers and the set of corresponding signals associated with each of the sex-associated glycosylation biomarkers. In various embodiments, the set of sex-associated glycosylation biomarkers comprises at least one of the sex-associated glycosylation biomarkers listed in Table 28. Table 28 below lists a group of peptide structures associated with sex.

In various embodiments, method S2900 may optionally include, at step S2930, generating the accuracy score based on the predicted sex and the annotated sex for each sample of the cohort.

In various embodiments, method S2900 may optionally include, at step S2940, generating a sensitivity score based on the predicted sex and the annotated sex for each sample of the cohort. The generated sensitivity score indicates a true positive rate when comparing the predicted sex and the annotated sex, in accordance with various embodiments. In various embodiments, the sensitivity score may be proportional to actual positive cases that are predicted as positive by the model. The sensitivity score may illustrate an overall performance of the model on the cohort of samples. A positive case occurs when the annotated sex is the same gender as the predicted sex.

In various embodiments, method S2900 may optionally include, at step S2950, generating a specificity score based on the predicted sex and the annotated sex for each sample of the cohort. The generated specificity score indicates a true negative rate when comparing the predicted sex and the annotated sex, in accordance with various embodiments. In various embodiments, the specificity score may be proportional to actual negative cases that are predicted as negative by the model. A negative case occurs when the annotated sex is not the same gender as the predicted sex. The specificity score may illustrate an overall performance of the model on the cohort of samples.

In various embodiments, each sample of the cohort has a disease condition, where the disease condition is selected from the group consisting of non-small cell lung cancer, breast cancer, pancreatic cancer, colorectal cancer, and nonalcoholic steatohepatitis (NASH). In various embodiments, each sample of the cohort has either a disease condition or a healthy condition, where the disease condition selected from the group consisting of non-small cell lung cancer, breast cancer, pancreatic cancer, colorectal cancer, and nonalcoholic steatohepatitis (NASH). In various embodiments, each sample of the cohort has either a single disease condition or a healthy condition where the disease conditions include non-small cell lung cancer, breast cancer, pancreatic cancer, colorectal cancer, and nonalcoholic steatohepatitis (NASH). In various embodiments, at least one of the sex-associated glycosylation biomarkers includes a glycopeptide structure defined by a peptide sequence and a glycan structure linked to the peptide sequence at a linking site of the peptide sequence, as identified in Table 28, with the peptide sequence being one of SEQ ID NOS: 183-196 as defined in Table 30A and 30B.

In various embodiments, method S2900 can optionally include receiving the peptide structure data for each sample of the cohort from a mass spectrometer. In various embodiments, the peptide structure data include at least one of a raw abundance, an adjusted raw abundance, a peptide concentration, a glycopeptide concentration, or a normalized concentration. In various embodiments, the peptide structure data comprise normalized concentration data, wherein the normalized concentration data is a function of at least one of peptide abundance data, corresponding internal standard abundance data, a spike-in concentration value, and a dilution factor. In various embodiments, the peptide structure data are generated using multiple reaction monitoring mass spectrometry (MRM-MS).

In various embodiments, method S2900 can optionally include creating a sample from the biological sample, and preparing the sample using reduction, alkylation, and enzymatic digestion to form a prepared sample that includes a set of peptide structures. In various embodiments, method S2900 can optionally include generating the peptide structure data from the prepared sample using multiple reaction monitoring mass spectrometry (MRM-MS).

In various embodiments, the set of sex-associated glycosylation biomarkers can include at least three of the sex-associated glycosylation biomarkers listed in Table 28. In various embodiments, the set of sex-associated glycosylation biomarkers can include at least five of the sex-associated glycosylation biomarkers listed in Table 28. One or more features (e.g., relative abundance, concentration, site occupancy) of these peptide structures may be used in a supervised machine learning model described elsewhere herein to generate an age-related indicator.

Table 28: Peptide Structures Associated with Sex

TABLE 28
Peptide Structures Associated with Sex

| | | | | | | Linking | Linking | |
|---|---|---|---|---|---|---|---|---|
| | | | | | | Site | Site | |
| | | | | | | Position | Position | |
| | | | | (Protein) | (Peptide) | within | within | Glycan |
| | PS-ID | | Uniprot | SEQ ID | SEQ ID | Protein | Peptide | Structure |
| PS-NAME | NO. | Protein Name | ID | NO. | NO. | Sequence | Sequence | GL NO. |
| IGJ (71) - 5401 | 123 | Immunoglobulin J chain | P01591 | 175 | 183 | 71 | 2 | 5401 |
| SHBG (380) - 5402 | 124 | Sex hormone-binding globulin | P04278 | 176 | 184 | 380 | 8 | 5402 |
| AACT (106) - 7614 | 125 | Alpha-1-antichymotrypsin | P01011 | 177 | 185 | 106 | 2 | 7614 |
| CO6 (324) - 5402 | 126 | Complement component C6 | P13671 | 178 | 186 | 324 | 3 | 5402 |
| CERU (138) - 5412 | 127 | Ceruloplasmin | P00450 | 179 | 187 | 138 | 10 | 5412 |
| IGJ (71MC) - 5412 | 128 | Immunoglobulin J chain | P01591 | 175 | 188 | 71 | 10 | 5412 |
| AACT (106) - 7604 | 129 | Alpha-1-antichymotrypsin | P01011 | 177 | 185 | 106 | 2 | 7604 |
| IGJ (71MC) - 5401 | 130 | Immunoglobulin J chain | P01591 | 175 | 188 | 71 | 10 | 5401 |
| AACT (106) - 7624 | 131 | Alpha-1-antichymotrypsin | P01011 | 177 | 185 | 106 | 2 | 7624 |
| AACT (106) - 6513 | 132 | Alpha-1-antichymotrypsin | P01011 | 177 | 185 | 106 | 2 | 6513 |
| KNG1 (169) - 5402 | 133 | Kininogen-1 | P01042 | 180 | 189 | 169 | 8 | 5402 |
| IGJ (71) - 5412 | 134 | Immunoglobulin J chain | P01591 | 175 | 183 | 71 | 2 | 5412 |
| KNG1 (169) - 6503 | 135 | Kininogen-1 | P01042 | 180 | 189 | 169 | 8 | 6503 |
| CERU (358) - | 136 | Ceruloplasmin | P00450 | 179 | 190 | n/a | n/a | NONGLYCOSYLATED |
| NONGLYCOSYLATED | | | | | | | | |
| CERU (358) - 5412 | 137 | Ceruloplasmin | P00450 | 179 | 190 | 358 | 13 | 5412 |
| KLKB1 (453) - 5402 | 138 | Plasma Kallikrein | P03952 | 181 | 191 | 453 | 7 | 5402 |
| CERU (138) - 5402 | 139 | Ceruloplasmin | P00450 | 179 | 187 | 138 | 10 | 5402 |
| ANGT (47) - 5401 | 140 | Angiotensinogen | P01019 | 182 | 192 | 47 | 12 | 5401 |
| ANGT (47) - 5402 | 141 | Angiotensinogen | P01019 | 182 | 192 | 47 | 12 | 5402 |
| KLKB1 (308) - 5401 | 142 | Plasma Kallikrein | P03952 | 181 | 193 | 308 | 13 | 5401 |
| CERU (138) - 6513 | 143 | Ceruloplasmin | P00450 | 179 | 187 | 138 | 10 | 6513 |
| IGJ (71MC) - 5411 | 144 | Immunoglobulin J chain | P01591 | 175 | 188 | 71 | 10 | 5411 |
| IGJ (71MC) - | 145 | Immunoglobulin J chain | P01591 | 175 | 188 | n/a | n/a | NONGLYCOSYLATED |
| NONGLYCOSYLATED | | | | | | | | |
| CERU (138) - 6503 | 146 | Ceruloplasmin | P00450 | 179 | 187 | 138 | 10 | 6503 |
| AACT (106) - 6503 | 147 | Alpha-1-antichymotrypsin | P01011 | 177 | 185 | 106 | 2 | 6503 |
| KLKB1 (453) - | 148 | Plasma Kallikrein | P03952 | 181 | 191 | n/a | n/a | NONGLYCOSYLATED |
| NONGLYCOSYLATED | | | | | | | | |
| CO6 (855) - 5402 | 149 | Complement component C6 | P13671 | 178 | 194 | 855 | 4 | 5402 |
| CERU (358) - 5402 | 150 | Ceruloplasmin | P00450 | 179 | 190 | 358 | 13 | 5402 |
| KLKB1 (494MC) - 5402 | 151 | Plasma Kallikrein | P03952 | 181 | 195 | 494 | 6 | 5402 |
| KLKB1 (494) - 5410 | 152 | Plasma Kallikrein | P03952 | 181 | 196 | 494 | 6 | 5410 |
| KLKB1 (494) - 5401 | 153 | Plasma Kallikrein | P03952 | 181 | 196 | 494 | 6 | 5401 |
| KLKB1 (494) - 5400 | 154 | Plasma Kallikrein | P03952 | 181 | 196 | 494 | 6 | 5400 |
| KLKB1 (494) - 6503 | 155 | Plasma Kallikrein | P03952 | 181 | 196 | 494 | 6 | 6503 |
| AACT (106) - 5402 | 156 | Alpha-1-antichymotrypsin | P01011 | 177 | 185 | 106 | 2 | 5402 |
| AACT (106) - 7603 | 157 | Alpha-1-antichymotrypsin | P01011 | 177 | 185 | 106 | 2 | 7603 |
| KLKB1 (494MC) - 6503 | 158 | Plasma Kallikrein | P03952 | 181 | 195 | 494 | 6 | 5502 |
| IGJ (71) - 5411 | 159 | Immunoglobulin J chain | P01591 | 175 | 183 | 71 | 2 | 5411 |
| KLKB1 (308) - 5402 | 160 | Plasma Kallikrein | P03952 | 181 | 193 | 308 | 13 | 5402 |
| KLKB1 (494) - 5402 | 161 | Plasma Kallikrein | P03952 | 181 | 196 | 494 | 6 | 5402 |
| CERU (138) - 6502 | 162 | Ceruloplasmin | P00450 | 179 | 187 | 138 | 10 | 6502 |
| KLKB1 (494MC) - 5401 | 163 | Plasma Kallikrein | P03952 | 181 | 195 | 494 | 6 | 5400 |
| KNG1 (169) - 6513 | 164 | Kininogen-1 | P01042 | 180 | 189 | 169 | 8 | 6513 |

Table 28 includes the Peptide Structure Identification Number (PS-ID NO.) that is a reference number for a particular peptide or glycopeptide. The Peptide Structure Name (PS-Name, e.g., IGJ (71)-5401), which is a reference code for the protein name (e.g., IGJ), followed by the glycan linking site position in the protein (e.g., the number 71 that is within a parenthesis and represents a sequential amino acid position in protein IGJ), and followed by the glycan structure GL number (e.g., the number 5402 that is preceded by a hyphen and represents a glycan composition Hex(5)HexNAc(4)Fuc(0)NeuAc(1)). The Protein Sequence ID No of Table 28 corresponds to the corresponding protein name, Unitprot ID, and amino acid sequence of Table 31. The Peptide Sequence ID No of Table 1 corresponds to the corresponding peptide sequence of Table 30A. The term Linking Site Pos. within Protein Sequence is a number that refers to the sequential position of an amino acid of the corresponding protein in which a glycan is attached. For the Glycan Linking Site Pos. within Protein Sequence, the amino acid position of the peptide sequence is defined by the sequentially numbered order of amino acids based on the Uniprot ID of the corresponding protein for the peptide sequence. The term Linking Site Pos. within Peptide Sequence is a number that refers to the sequential position of an amino acid of the corresponding peptide in which a glycan is attached. For the Glycan Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Table 32.

In some embodiments, the term AGP12 represents that the glycopeptide is a fragment of either AGP1 or AGP2. In some embodiments, the term IGA12 represents that the glycopeptide is a fragment of either IGA1 or IGA2.

In some instances of the Peptide Structure (PS) NAME, subsequent to the prefix, there is a number noted with the notation MC that indicates that there was a missed cleavage at position in the peptide sequence as noted by the number. In some instances of the Peptide Structure (PS) NAME, there is a suffix NH₃LOSS to indicate a loss of a NH₃ group.

### XIV.B. Training the Model for Quality Control of Samples

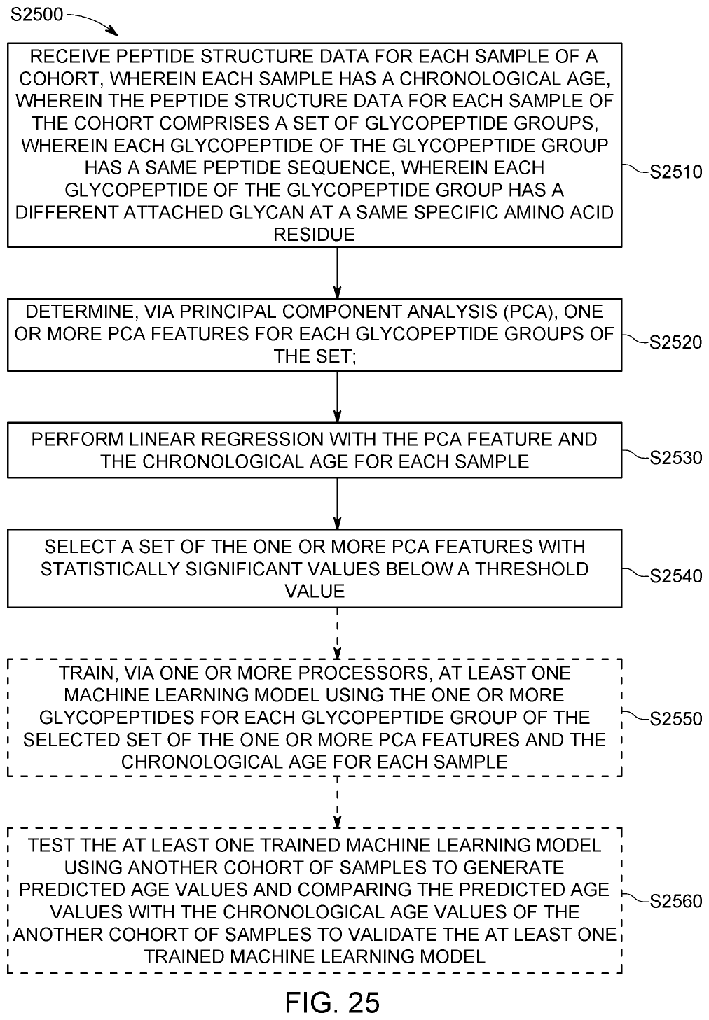

FIG. 25 is a flowchart of method S2500 of generating a model to predict an age of a patient, in accordance with various embodiments. Method S2500 may be implemented using, for example, at least a portion of workflow 100 as described in FIGS. 1, 2, and/or analysis system 300 as described in FIG. 3. In some embodiments, method S2500 may be one example of an implementation for training the model used in the method S2400 in FIG. 24.

As illustrated in FIG. 25, method S2500 includes, at step S2510, receiving peptide structure data for each sample of a cohort, wherein each sample has a chronological age, wherein the peptide structure data for each sample of the cohort include a set of glycopeptide groups, wherein each glycopeptide of the glycopeptide group has a same peptide sequence, and wherein each glycopeptide of the glycopeptide group has a different attached glycan at a same specific amino acid residue. At step S2520, method S2500 further includes determining, via principal component analysis (PCA), such as PCA 318, one or more PCA features, such as PCA features 319, for each glycopeptide group of the set of glycopeptide groups.

Method S2500 further includes, at step S2530, performing linear regression with the PCA feature and the chronological age for each sample, and at step S2540, selecting a set of the one or more PCA features with statistically significant values below a threshold value. In various embodiments, the statistically significant values for each of the PCA features is an output of the performed linear regression. In various embodiments, each of the PCA features are associated with a glycopeptide group. In various embodiments, each of the glycopeptide groups may include one or more glycopeptides where the one or more glycopeptides are glycoforms.

As illustrated in FIG. 25, method S2500 may optionally include, at step S2550, training, via one or more processors, at least one machine learning model using the one or more glycopeptides for each glycopeptide group of the selected set of the one or more PCA features and the chronological age for each sample. In various embodiments, training data can be used for training the supervised machine learning model. The training data can include a plurality of peptide structure profiles for a plurality of subjects. In various embodiments, method S2500 may optionally include, at step S2560, testing the at least one trained machine learning model using another cohort of samples to generate predicted age values and comparing the predicted age values with the chronological age values of the another cohort of samples to validate the at least one trained machine learning model. The trained machine learning model can include ElasticNet. In some embodiments, the machine learning model can include a binary classification model. Some binary classification models can include logistical regression models. Some logistical regression models can include LASSO regression models.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that selects the peptide structures identified in Table 23. The markers used for training of the LASSO regression model may, in one or more embodiments, additionally include one or more other peptide structure markers.

In various embodiments of method S2500, each sample of the cohort has a disease condition, the disease condition selected from the group consisting of non-small cell lung cancer, breast cancer, pancreatic cancer, colorectal cancer, and nonalcoholic steatohepatitis (NASH). In various embodiments of method S2500, each sample of the cohort has either a disease condition or a healthy condition, the disease condition selected from the group consisting of non-small cell lung cancer, breast cancer, pancreatic cancer, colorectal cancer, and nonalcoholic steatohepatitis (NASH). In various embodiments of method S2500, each sample of the cohort has either a single disease condition or a healthy condition.

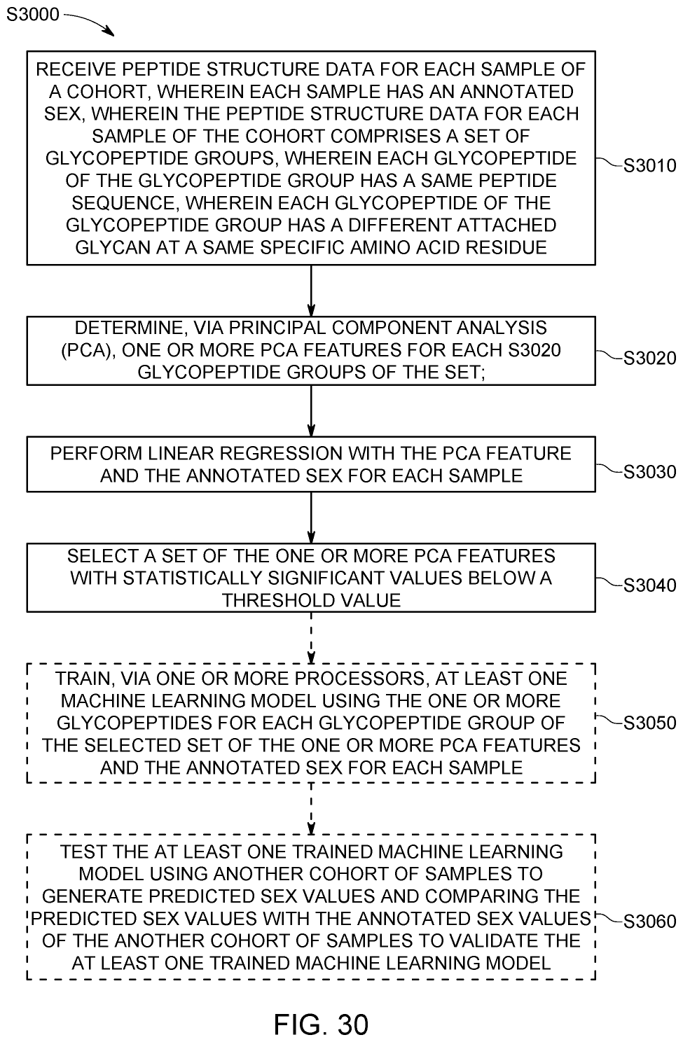

FIG. 30 is a flowchart of method S3000 of generating a model to predict a sex of a patient, in accordance with various embodiments. Method S3000 may be implemented using, for example, at least a portion of workflow 100 as described in FIGS. 1, 2, and/or analysis system 300 as described in FIG. 3. In some embodiments, method S3000 may be one example of an implementation for training the model used in the method S2900 in FIG. 29.

As illustrated in FIG. 30, method S3000 includes, at step S3010, receiving peptide structure data for each sample of a cohort, wherein each sample has an annotated sex, wherein the peptide structure data for each sample of the cohort include a set of glycopeptide groups, wherein each glycopeptide of the glycopeptide group has a same peptide sequence, and wherein each glycopeptide of the glycopeptide group has a different attached glycan at a same specific amino acid residue. At step S3020, method S3000 further includes determining, via principal component analysis (PCA), such as PCA 318, one or more PCA features, such as PCA features 319, for each glycopeptide group of the set of glycopeptide groups.

Method S3000 further includes, at step S3030, performing linear regression with the PCA feature and the annotated sex for each sample, and at step S3040, selecting a set of the one or more PCA features with statistically significant values below a threshold value. In various embodiments, the statistically significant values for each of the PCA features is an output of the performed linear regression. In various embodiments, each of the PCA features are associated with a glycopeptide group. In various embodiments, each of the glycopeptide groups may include one or more glycopeptides where the one or more glycopeptides are glycoforms.

As illustrated in FIG. 30, method S3000 may optionally include, at step S3050, training, via one or more processors, at least one machine learning model using the one or more glycopeptides for each glycopeptide group of the selected set of the one or more PCA features and the annotated sex for each sample. In various embodiments, training data can be used for training the supervised machine learning model. The training data can include a plurality of peptide structure profiles for a plurality of subjects. In various embodiments, method S3000 may optionally include, at step S3060, testing the at least one trained machine learning model using another cohort of samples to generate predicted sex values and comparing the predicted sex values with the annotated sex values of the another cohort of samples to validate the at least one trained machine learning model. The trained machine learning model can include ElasticNet. In some embodiments, the machine learning model can include a binary classification model. Some binary classification models can include logistical regression models. Some logistical regression models can include LASSO regression models.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be an elastic regression, logistical regression, or LASSO regression model that selects the peptide structures identified in Table 28. The markers used for training one of the regression models may, in one or more embodiments, additionally include one or more other peptide structure markers.

In various embodiments of method S3000, each sample of the cohort has a disease condition, the disease condition selected from the group consisting of non-small cell lung cancer, breast cancer, pancreatic cancer, colorectal cancer, and nonalcoholic steatohepatitis (NASH). In various embodiments of method S3000, each sample of the cohort has either a disease condition or a healthy condition, the disease condition selected from the group consisting of non-small cell lung cancer, breast cancer, pancreatic cancer, colorectal cancer, and nonalcoholic steatohepatitis (NASH). In various embodiments of method S3000, each sample of the cohort has either a single disease condition or a healthy condition.

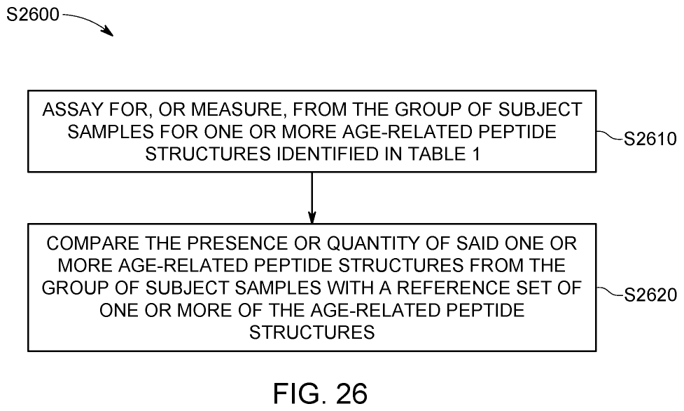

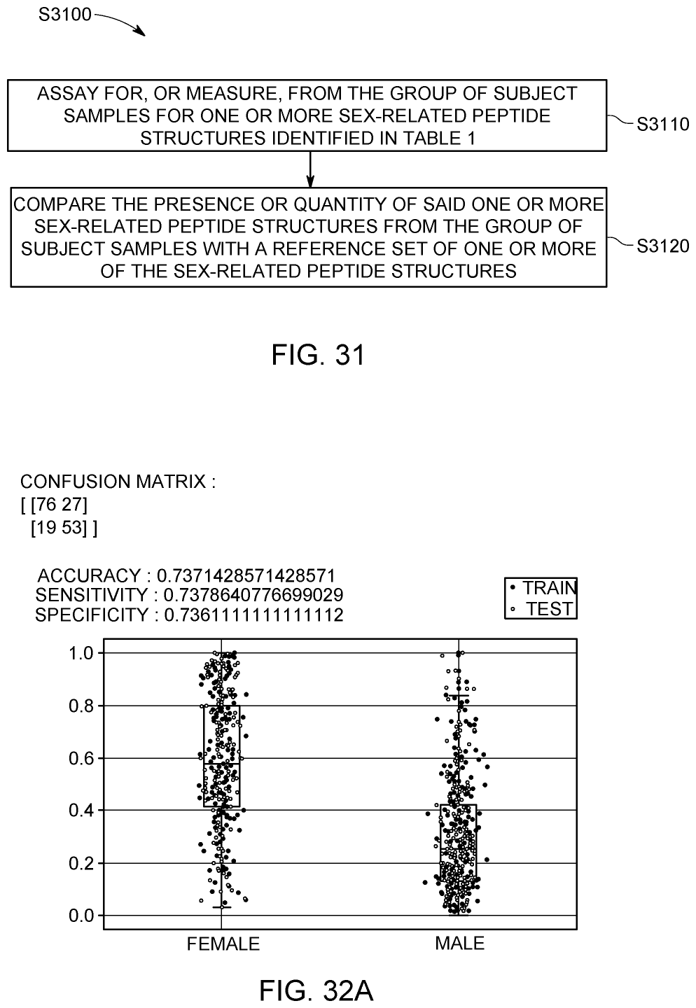

### XIV.C. Performing Quality Control of Samples

[1] FIG. 26 is a flowchart of method S2600 of performing quality control of samples, in accordance with various embodiments. FIG. 31 is a flowchart of method S3100 of performing quality control of samples, in accordance with various embodiments

### I. Peptide Structure and Product Ion Compositions, Kits and Reagents

Aspects of the disclosure include compositions comprising one or more of the peptide structures listed in Table 23. In some embodiments, a composition comprises a plurality of the peptide structures listed in Table 23. In some embodiments, a composition comprises 1, 2, 3, 4, 5, 6, 7, 8, 9, 10, 11, 12, 13, 14, 15, 16, 17, 18, 19, or all 20 of the peptide structures listed in Table 23. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 163-174, listed in Table 23. In various embodiments, the peptide structures listed in Table 23 are in accordance with the peptide sequences listed in Table 25A and the glycan symbol structures and glycan composition of Table 27.

Aspects of the disclosure include compositions comprising one or more precursor ions having a defined charge and/or defined mass-to-charge (m/z) ratio, as listed in Table 25. Aspects of the disclosure include compositions comprising one or more product ions having a defined mass-to-charge (m/z) ratio, which product ions are produced by converting a peptide structure described herein (e.g., a peptide structure listed in Table 23) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 23). In some embodiments, a composition comprises a set of the product ions listed in Table 24, having an m/z ratio selected from the list provided for each peptide structure in Table 23 or Table 24.

In some embodiments, a composition comprises at least one of peptide structures PS-103, PS-104, PS-105, PS-106, PS-107, PS-108, PS-109. PS-110, PS-111, PS-112, PS-113, PS-114, PS-115, PS-116, PS-117, PS-118, PS-119, PS-120, PS-121, and PS-122 identified in Table 23. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or all 20 of the peptide structures PS-103, PS-104, PS-105, PS-106, PS-107, PS-108, PS-109. PS-110, PS-111, PS-112, PS-113, PS-114, PS-115, PS-116, PS-117, PS-118, PS-119, PS-120, PS-121, and PS-122 in Table 23.

In some embodiments, a composition comprises a peptide structure or a product ion. The peptide structure or product ion comprises an amino acid sequence having at least 90% sequence identity to any one of SEQ ID NOS: 163-174, as identified in Table 25A, corresponding to peptide structures PS-103, PS-104, PS-105, PS-106, PS-107, PS-108, PS-109. PS-110, PS-111, PS-112, PS-113, PS-114, PS-115, PS-116, PS-117, PS-118, PS-119, PS-120, PS-121, and PS-122 in Table 23.

In some embodiments, the product ion is selected as one from a group consisting of product ions identified in Table 24, including product ions falling within an identified m/z range of the m/z ratio identified in Table 24 and characterized as having a precursor ion having an m/z ratio within an identified m/z range of the m/z ratio identified in Table 24. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (+1.5). Thus, a composition may include a product ion having an m/z ratio that falls within at least one of the first range (+0.5), the second range (+0.8), or the third range (1.0) of the product ion m/z ratio identified in Table 25, and characterized as having a precursor ion having an m/z ratio that falls within at least one of first range (+0.5), a second range (1.0), or a third range (1.0 of the precursor ion m/z ratio identified in Table 24.

TABLE 24
Mass Spectrometry-Related Characteristics for the Peptide
Structures associated with Age or a Range of Ages

| | | | | | | | Colli- |
|---|---|---|---|---|---|---|---|
| | Mono- | | | | 2nd | | sion |
| PS- | isotopic | Pre- | | Prod- | Prod- | | En- |
| ID | mass | cursor | Precurs | uct | uct | RT | ergy- |
| NO. | (g/mol) | m/z | charge | m/z | m/z | (Min) | Volt |
| 103 | 4517.13034 | 1130.8 | 4 | 204.1 | 1258.7 | 39.4 | 35 |
| 104 | 5107.14298 | 1278.3 | 4 | 204.1 | 1152.6 | 35.7 | 40 |
| 105 | 2957.14418 | 987.1 | 3 | 366.1 | 1392.6 | 7.8 | 24 |
| 106 | 4019.838876 | 1341.6 | 3 | 204.1 | 1281.2 | 41.66 | 25 |
| 107 | 3342.25967 | 1115.1 | 3 | 366.1 | 1341.6 | 10.6 | 30 |
| 108 | 2836.117908 | 946.5 | 3 | 204.1 | 1392.6 | 8.1 | 15 |
| 109 | 3128.23372 | 1043.8 | 3 | 366.1 | n/a | 12.8 | 25 |
| 110 | 3248.23959 | 1084.1 | 3 | 366.1 | 1392.6 | 8.2 | 27 |
| 111 | 5755.448988 | 1152.7 | 5 | 366.1 | 1550.3 | 41 | 28 |
| 112 | 2998.170728 | 1000.7 | 3 | 204.1 | 1392.6 | 8 | 15 |
| 113 | 4961.085074 | 1241.8 | 4 | 204.1 | 1152.6 | 35.8 | 35 |
| 114 | 4718.889192 | 1181.1 | 4 | 366.1 | n/a | 32.7 | 29 |
| 115 | 3220.36373 | 1074.5 | 3 | 366.1 | n/a | 13.1 | 24 |
| 116 | 6266.639082 | 1568.4 | 4 | 366.1 | 366.1 | 40.9 | 30 |
| 117 | 4562.887832 | 1142.2 | 4 | 366.1 | n/a | 35.9 | 20 |
| 118 | 5167.363312 | 1292.9 | 4 | 366.1 | n/a | 41.5 | 32 |
| 119 | 2633.03854 | 879 | 3 | 204.1 | 1392.6 | 7.9 | 21 |
| 120 | 4397.80301 | 1467 | 3 | 366.1 | n/a | 25.3 | 30 |
| 121 | 3539.335 | 1181.1 | 3 | 204.1 | n/a | 8.6 | 40 |
| 122 | 2690.060002 | 898 | 3 | 1392.6 | n/a | 8.1 | 25 |

Table 25A defines the peptide sequences for SEQ ID NOS: 163-174 from Table 23. Table 26 further identifies a corresponding protein SEQ ID NO. for each peptide sequence.

TABLE 25A
Peptide SEQ ID NOS

| Prot | | Pept |
|---|---|---|
| SEQ | | SEQ ID |
| ID NO. | Peptide.sequence | NO. |
| 153 | VGQLQLSHNLSLVILVPQNLK | 163 |
| 153 | VLSNNSDANLELINTWVAK | 164 |
| 154 | EEQYNSTYR | 165 |
| 155 | PALEDLLLGSEANLTCTLTGLR | 166 |
| 156 | FGCEIENNR | 167 |
| 154 | EEQYNSTYR | 165 |
| 157 | EEQFNSTFR | 168 |
| 154 | EEQYNSTYR | 165 |
| 158 | SVQEIQATFFYFTPNKTEDTIFLR | 169 |
| 154 | EEQYNSTYR | 165 |
| 153 | VLSNNSDANLELINTWVAK | 164 |
| 159 | QQQHLFGSNVTDCSGNFCLFR | 170 |
| 160 | TPLTANITK | 171 |
| 158 | SVQEIQATFFYFTPNKTEDTIFLR | 169 |
| 158 | SVQEIQATFFYFTPNK | 172 |
| 155 | LSLHRPALEDLLLGSEANLTCTLTGLR | 173 |
| 154 | EEQYNSTYR | 165 |
| 161 | GLTFQQNASSMCVPDQDTAIR | 174 |
| 154 | EEQYNSTYR | 165 |
| 154 | EEQYNSTYR | 165 |

Table 25B provides an indication of particular markers and includes the starting position of the peptide sequence within the protein sequence and the end position of the peptide sequence within the protein sequence.

TABLE 25B
Markers and Protein Positions

| PS-ID | | | Start | End |
|---|---|---|---|---|
| NO. | PS-NAME | Peptide.sequence | position | position |
| 103 | IC1 (352)-5402 | VGQLQLSHNLSLVILVPQNLK | 344 | 364 |
| 104 | IC1 (253)-6513 | VLSNNSDANLELINTWVAK | 250 | 268 |
| 105 | IGG1 (297)-5410 | EEQYNSTYR | 293 | 301 |
| 106 | IGA12 (144MC)-4500 | PALEDLLLGSEANLTCTLTGLR | 132 | 153 |
| 107 | ZA2G (128)-5402 | FGCEIENNR | 121 | 129 |
| 108 | IGG1 (297)-3510 | EEQYNSTYR | 293 | 301 |
| 109 | IGG2 (297)-5510 | EEQFNSTFR | 293 | 301 |
| 110 | IGG1 (297)-5411 | EEQYNSTYR | 293 | 301 |
| 111 | AGP12 (72MC)-6503 | SVQEIQATFFYFTPNKTEDTIFLR | 58 | 81 |
| 112 | IGG1 (297)-4510 | EEQYNSTYR | 293 | 301 |
| 113 | IC1 (253)-6503 | VLSNNSDANLELINTWVAK | 250 | 268 |
| 114 | TRFE (630)-5402 | QQQHLFGSNVTDCSGNFCLFR | 622 | 642 |
| 115 | IGA2 (205)-5511 | TPLTANITK | 200 | 208 |
| 116 | AGP12 (72MC)-7613 | SVQEIQATFFYFTPNKTEDTIFLR | 58 | 81 |
| 117 | AGP12 (72)-7601 | SVQEIQATFFYFTPNK | 58 | 73 |
| 118 | IGA12 (144)-5402 | LSLHRPALEDLLLGSEANLTCTLTGLR | 127 | 153 |
| 119 | IGG1 (297)-3410 | EEQYNSTYR | 293 | 301 |
| 120 | IGM (209)-5411_Z3 | GLTFQQNASSMCVPDQDTAIR | 203 | 223 |
| 121 | IGG1 (297)-5412 | EEQYNSTYR | 293 | 301 |
| 122 | IGG1 (297)-3500 | EEQYNSTYR | 293 | 301 |

Table 26 identifies the proteins of SEQ ID NOS: 153-161 from Table 23. Table 26 identifies a corresponding protein abbreviation, protein name, and amino acid sequence for each of protein SEQ ID NOS: 153-161. Further, Table 26 identifies a corresponding Uniprot ID for each of protein SEQ ID NOS: 153-161.

TABLE 26
Protein SEQ ID NOS

| | | | Prot | |
|---|---|---|---|---|
| | | | SEQ | |
| Protein | Protein | Uniprot | ID | |
| Abbrev. | Name | ID | NO | Protein Sequence |
| IC1 | Plasma | P05155 | 153 | MASRLTLLTLLLLLLAGDRASSNPNATSSSSQDPESLQ |
| | protease C1 | | | DRGEGKVATTVISKMLFVEPILEVSSLPTTNSTTNSAT |
| | inhibitor | | | KITANTTDEPTTQPTTEPTTQPTIQPTQPTTQLPTDSPT |
| | | | | QPTTGSFCPGPVTLCSDLESHSTEAVLGDALVDFSLKL |
| | | | | YHAFSAMKKVETNMAFSPFSIASLLTQVLLGAGENTK |
| | | | | TNLESILSYPKDFTCVHQALKGFTTKGVTSVSQIFHSP |
| | | | | DLAIRDTFVNASRTLYSSSPRVLSNNSDANLELINTWV |
| | | | | AKNTNNKISRLLDSLPSDTRLVLLNAIYLSAKWKTTFD |
| | | | | PKKTRMEPFHFKNSVIKVPMMNSKKYPVAHFIDQTLK |
| | | | | AKVGQLQLSHNLSLVILVPQNLKHRLEDMEQALSPSV |
| | | | | FKAIMEKLEMSKFQPTLLTLPRIKVTTSQDMLSIMEKL |
| | | | | EFFDFSYDLNLCGLTEDPDLQVSAMQHQTVLELTETG |
| | | | | VEAAAASAISVARTLLVFEVQQPFLFVLWDQQHKFPV |
| | | | | FMGRVYDPRA |
| IGG1 | Immunoglobulin | P01857 | 154 | ASTKGPSVFPLAPSSKSTSGGTAALGCLVKDYFPEPVT |
| | heavy | | | VSWNSGALTSGVHTFPAVLQSSGLYSLSSVVTVPSSSL |
| | constant | | | GTQTYICNVNHKPSNTKVDKKVEPKSCDKTHTCPPCP |
| | gamma 1 | | | APELLGGPSVFLFPPKPKDTLMISRTPEVTCVVVDVSH |
| | | | | EDPEVKFNWYVDGVEVHNAKTKPREEQYNSTYRVVS |
| | | | | VLTVLHQDWLNGKEYKCKVSNKALPAPIEKTISKAKG |
| | | | | QPREPQVYTLPPSRDELTKNQVSLTCLVKGFYPSDIAV |
| | | | | EWESNGQPENNYKTTPPVLDSDGSFFLYSKLTVDKSR |
| | | | | WQQGNVFSCSVMHEALHNHYTQKSLSLSPGK |
| IGA1 | Immunoglobulin | P01876 | 155 | ASPTSPKVFPLSLCSTQPDGNVVIACLVQGFFPQEPLSV |
| | heavy | | | TWSESGQGVTARNFPPSQDASGDLYTTSSQLTLPATQ |
| | constant | | | CLAGKSVTCHVKHYTNPSQDVTVPCPVPSTPPTPSPST |
| | alpha 1 | | | PPTPSPSCCHPRLSLHRPALEDLLLGSEANLTCTLTGLR |
| | | | | DASGVTFTWTPSSGKSAVQGPPERDLCGCYSVSSVLP |
| | | | | GCAEPWNHGKTFTCTAAYPESKTPLTATLSKSGNTFR |
| | | | | PEVHLLPPPSEELALNELVTLTCLARGFSPKDVLVRWL |
| | | | | QGSQELPREKYLTWASRQEPSQGTTTFAVTSILRVAA |
| | | | | EDWKKGDTFSCMVGHEALPLAFTQKTIDRLAGKPTH |
| | | | | VNVSVVMAEVDGTCY |
| ZA2G | Zinc-alpha- | P25311 | 156 | MVRMVPVLLSLLLLLGPAVPQENQDGRYSLTYIYTGL |
| | 2- | | | SKHVEDVPAFQALGSLNDLQFFRYNSKDRKSQPMGL |
| | glycoprotein | | | WRQVEGMEDWKQDSQLQKAREDIFMETLKDIVEYY |
| | | | | NDSNGSHVLQGRFGCEIENNRSSGAFWKYYYDGKDY |
| | | | | IEFNKEIPAWVPFDPAAQITKQKWEAEPVYVQRAKAY |
| | | | | LEEECPATLRKYLKYSKNILDRQDPPSVVVTSHQAPG |
| | | | | EKKKLKCLAYDFYPGKIDVHWTRAGEVQEPELRGDV |
| | | | | LHNGNGTYQSWVVVAVPPQDTAPYSCHVQHSSLAQP |
| | | | | LVVPWEAS |
| IGG2 | Immunoglobulin | P01859 | 157 | ASTKGPSVFPLAPCSRSTSESTAALGCLVKDYFPEPVT |
| | heavy | | | VSWNSGALTSGVHTFPAVLQSSGLYSLSSVVTVPSSNF |
| | constant | | | GTQTYTCNVDHKPSNTKVDKTVERKCCVECPPCPAPP |
| | gamma 2 | | | VAGPSVFLFPPKPKDTLMISRTPEVTCVVVDVSHEDPE |
| | | | | VQFNWYVDGVEVHNAKTKPREEQFNSTFRVVSVLTV |
| | | | | VHQDWLNGKEYKCKVSNKGLPAPIEKTISKTKGQPRE |
| | | | | PQVYTLPPSREEMTKNQVSLTCLVKGFYPSDISVEWES |
| | | | | NGQPENNYKTTPPMLDSDGSFFLYSKLTVDKSRWQQ |
| | | | | GNVFSCSVMHEALHNHYTQKSLSLSPGK |
| AGP1 | Alpha-1- | P02763 | 158 | MALSWVLTVLSLLPLLEAQIPLCANLVPVPITNATLDR |
| | acid | | | ITGKWFYIASAFRNEEYNKSVQEIQATFFYFTPNKTED |
| | glycoprotein | | | TIFLREYQTRQDQCIYNTTYLNVQRENGTISRYVGGQE |
| | 1 | | | HFAHLLILRDTKTYMLAFDVNDEKNWGLSVYADKPE |
| | | | | TTKEQLGEFYEALDCLRIPKSDVVYTDWKKDKCEPLE |
| | | | | KQHEKERKQEEGES |
| TRFE | Sero- | P02787 | 159 | MRLAVGALLVCAVLGLCLAVPDKTVRWCAVSEHEA |
| | transferrin | | | TKCQSFRDHMKSVIPSDGPSVACVKKASYLDCIRAIA |
| | | | | ANEADAVTLDAGLVYDAYLAPNNLKPVVAEFYGSKE |
| | | | | DPQTFYYAVAVVKKDSGFQMNQLRGKKSCHTGLGRS |
| | | | | AGWNIPIGLLYCDLPEPRKPLEKAVANFFSGSCAPCAD |
| | | | | GTDFPQLCQLCPGCGCSTLNQYFGYSGAFKCLKDGA |
| | | | | GDVAFVKHSTIFENLANKADRDQYELLCLDNTRKPV |
| | | | | DEYKDCHLAQVPSHTVVARSMGGKEDLIWELLNQAQ |
| | | | | EHFGKDKSKEFQLFSSPHGKDLLFKDSAHGFLKVPPR |
| | | | | MDAKMYLGYEYVTAIRNLREGTCPEAPTDECKPVKW |
| | | | | CALSHHERLKCDEWSVNSVGKIECVSAETTEDCIAKI |
| | | | | MNGEADAMSLDGGFVYIAGKCGLVPVLAENYNKSD |
| | | | | NCEDTPEAGYFAIAVVKKSASDLTWDNLKGKKSCHT |
| | | | | AVGRTAGWNIPMGLLYNKINHCRFDEFFSEGCAPGSK |
| | | | | KDSSLCKLCMGSGLNLCEPNNKEGYYGYTGAFRCLV |
| | | | | EKGDVAFVKHQTVPQNTGGKNPDPWAKNLNEKDYE |
| | | | | LLCLDGTRKPVEEYANCHLARAPNHAVVTRKDKEAC |
| | | | | VHKILRQQQHLFGSNVTDCSGNFCLFRSETKDLLFRD |
| | | | | DTVCLAKLHDRNTYEKYLGEEYVKAVGNLRKCSTSS |
| | | | | LLEACTFRRP |
| IGA2 | Immunoglobulin | P01877 | 160 | ASPTSPKVFPLSLDSTPQDGNVVVACLVQGFFPQEPLS |
| | heavy | | | VTWSESGQNVTARNFPPSQDASGDLYTTSSQLTLPAT |
| | constant | | | QCPDGKSVTCHVKHYTNSSQDVTVPCRVPPPPPCCHP |
| | alpha 2 | | | RLSLHRPALEDLLLGSEANLTCTLTGLRDASGATFTW |
| | | | | TPSSGKSAVQGPPERDLCGCYSVSSVLPGCAQPWNHG |
| | | | | ETFTCTAAHPELKTPLTANITKSGNTFRPEVHLLPPPSE |
| | | | | ELALNELVTLTCLARGFSPKDVLVRWLQGSQELPREK |
| | | | | YLTWASRQEPSQGTTTYAVTSILRVAAEDWKKGETFS |
| | | | | CMVGHEALPLAFTQKTIDRMAGKPTHINVSVVMAEA |
| | | | | DGTCY |
| IGM | Immunoglobulin | P01871 | 161 | GSASAPTLFPLVSCENSPSDTSSVAVGCLAQDFLPDSIT |
| | heavy | | | FSWKYKNNSDISSTRGFPSVLRGGKYAATSQVLLPSK |
| | constant | | | DVMQGTDEHVVCKVQHPNGNKEKNVPLPVIAELPPK |
| | mu | | | VSVFVPPRDGFFGNPRKSKLICQATGFSPRQIQVSWLR |
| | | | | EGKQVGSGVTTDQVQAEAKESGPTTYKVTSTLTIKES |
| | | | | DWLGQSMFTCRVDHRGLTFQQNASSMCVPDQDTAIR |
| | | | | VFAIPPSFASIFLTKSTKLTCLVTDLTTYDSVTISWTRQ |
| | | | | NGEAVKTHTNISESHPNATFSAVGEASICEDDWNSGE |
| | | | | RFTCTVTHTDLPSPLKQTISRPKGVALHRPDVYLLPPA |
| | | | | REQLNLRESATITCLVTGFSPADVFVQWMQRGQPLSP |
| | | | | EKYVTSAPMPEPQAPGRYFAHSILTVSEEEWNTGETY |
| | | | | TCVVAHEALPNRVTERTVDKSTGKPTLYNVSLVMSD |
| | | | | TAGTCY |
| AGP2 | Alpha-1- | P19652 | 162 | MALSWVLTVLSLLPLLEAQIPLCANLVPVPITNATLDR |
| | acid | | | ITGKWFYIASAFRNEEYNKSVQEIQATFFYFTPNKTED |
| | glycoprotein | | | TIFLREYQTRQNQCFYNSSYLNVQRENGTVSRYEGGR |
| | 2 | | | EHVAHLLFLRDTKTLMFGSYLDDEKNWGLSFYADKP |
| | | | | ETTKEQLGEFYEALDCLCIPRSDVMYTDWKKDKCEPL |
| | | | | EKQHEKERKQEEGES |

Table 27 identifies and defines the glycan structures included in Table 23, all of which are N-glycans. Table 27 identifies a coded representation of the composition for each glycan structure included in Table 23. As used herein, the 4-digit GL NO. is a designation that represents the number of hexoses, the number of HexNAcs, the number of Fucoses, and the number of Neuraminic Acids. Table 27 illustrates the symbol structure and composition of detected glycan moieties that correspond to glycopeptides of Table 23 based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. For reference, N-linked glycans have a glycan attached to the amino acid asparagine and O-linked glycans have a glycan attached to either a serine or a threonine.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

Referring back to Tables 5, for some entries, there are two symbol structures provided for one Glycan Structure GL NO such as, for example, Glycan Structure GL NO 4500. Thus, the identify of a peptide that references a Glycan Structure GL NO that has two symbol structures could be one of two possibilities based on the MRM of the LC-MS analysis. In some instances, a bracket symbol is used as part of the Symbol Structure (e.g., 7601) to indicate that the precise bonding linkage is not exactly known, but that the linking line segment is attached to one of the plurality of adjacent carbohydrates immediately adjacent to the bracket.

TABLE 27
Glycan structure GL NO, symbol structure, and composition

| Glycan | | | |
|---|---|---|---|
| Structure | Symbol | Glycan | Glycan_{—} |
| GL NO. | Structure | Composition | MW |
| 7601 | [symbol] | Hex(7)HexNAc(6)Fuc(0)NeuAc(1) | 2643.941358 |
| 6503 | [symbol] | Hex(6)HexNAc(5)Fuc(0)NeuAc(3) | 2860.99999 |
| 7613 | [symbol] | Hex(7)HexNAc(6)Fuc(1)NeuAc(3) | 3372.190084 |
| 6513 | [symbol] | Hex(6)HexNAc(5)Fuc(1)NeuAc(3) | 3007.057896 |
| 5402 | [symbol] | Hex(5)HexNAc(4)Fuc(0)NeuAc(2) | 2204.772392 |
| 4500 | [symbol] | Hex(4)HexNAc(5)Fuc(0)NeuAc(0) | 1825.66094 |
| 5511 | [symbol] | Hex(5)HexNAc(5)Fuc(1)NeuAc(1) | 2262.814256 |
| 3410 | [symbol] | Hex(3)HexNAc(4)Fuc(1)NeuAc(0) | 1444.533838 |
| 3500 | [symbol] | Hex(3)HexNAc(5)Fuc(0)NeuAc(0) | 1501.5553 |
| 3510 | [symbol] | Hex(3)HexNAc(5)Fuc(1)NeuAc(0) | 1647.613206 |
| 4510 | [symbol] | Hex(4)HexNAc(5)Fuc(1)NeuAc(0) | 1809.666026 |
| 5410 | [symbol] | Hex(5)HexNAc(4)Fuc(1)NeuAc(0) | 1768.639478 |
| 5411 | [symbol] | Hex(5)HexNAc(4)Fuc(1)NeuAc(1) | 2059.734888 |
| 5412 | [symbol] | Hex(5)HexNAc(4)Fuc(1)NeuAc(2) | 2350.830298 |
| 5510 | [symbol] | Hex(5)HexNAc(5)Fuc(1)NeuAc(0) | 1971.718846 |

Legend for Table 27:
● Glc [symbol]
Gal [symbol]
Man [symbol]
Fuc [symbol]
Neu5Ac
▪ GlcNAc [symbol]
GalNAc [symbol]
ManNAc

The identity of the various monosaccharides is illustrated by the Legend section located at the end of Table 27. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful as quality control compositions and data with respect to a group of samples. A transition includes a precursor ion and at least one product ion grouping. As encompassed herein, the peptide structures in Table 23, as well as their corresponding precursor ion and product ion groupings (these ions having defined m/z ratios or m/z ratios that fall within the m/z ranges identified herein) in Table 24, can be used in mass spectrometry-based analyses as positive controls for performance quality and/or accuracy.

Aspects of the disclosure include methods for analyzing one or more peptide structures, as described herein. In some embodiments, the methods involve processing a sample from a subject, such as a patient, to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 24 or an m/z ratio within an identified m/z ratio as provided in Table 24. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### II. Peptide Structure and Product Ion Compositions, Kits and Reagents

Aspects of the disclosure include compositions comprising one or more of the peptide structures listed in Table 28. In some embodiments, a composition comprises a plurality of the peptide structures listed in Table 28. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or all 42 of the peptide structures listed in Table 28. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 183-196, listed in Table 28. In various embodiments, the peptide structures listed in Table 28 are in accordance with the peptide sequences listed in Table 30A and the glycan symbol structures and glycan composition of Table 32.

Aspects of the disclosure include compositions comprising one or more precursor ions having a defined charge and/or defined mass-to-charge (m/z) ratio, as listed in Table 30. Aspects of the disclosure include compositions comprising one or more product ions having a defined mass-to-charge (m/z) ratio, which product ions are produced by converting a peptide structure described herein (e.g., a peptide structure listed in Table 28) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 28). In some embodiments, a composition comprises a set of the product ions listed in Table 29, having an m/z ratio selected from the list provided for each peptide structure in Table 28 or Table 29.

In some embodiments, a composition comprises at least one of peptide structures PS-123, PS-124, PS-125, PS-126, PS-127, PS-128, PS-129, PS-130, PS-131, PS-132, PS-133, PS-134, PS-135, PS-136, PS-137, PS-138, PS-139, PS-140, PS-141, PS-142, PS-143, PS-144, PS-145, PS-146, PS-147, PS-148, PS-149, PS-150, PS-151, PS-152, PS-153, PS-154, PS-155, PS-156, PS-157, PS-158, PS-159, PS-160, PS-161, PS-162, PS-163, and PS-164 identified in Table 28. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, or all 42 of the peptide structures PS-123, PS-124, PS-125, PS-126, PS-127, PS-128, PS-129, PS-130, PS-131, PS-132, PS-133, PS-134, PS-135, PS-136, PS-137, PS-138, PS-139, PS-140, PS-141, PS-142, PS-143, PS-144, PS-145, PS-146, PS-147, PS-148, PS-149, PS-150, PS-151, PS-152, PS-153, PS-154, PS-155, PS-156, PS-157, PS-158, PS-159, PS-160, PS-161, PS-162, PS-163, and PS-164 in Table 28.

In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, or all 42 of the precursor and product ions in Table 29.

In some embodiments, a composition comprises a peptide structure or a product ion. The peptide structure or product ion comprises an amino acid sequence having at least 90% sequence identity to any one of SEQ ID NOS: 183-196, as identified in Table 30A, corresponding to peptide structures PS-123, PS-124, PS-125, PS-126, PS-127, PS-128, PS-129, PS-130, PS-131, PS-132, PS-133, PS-134, PS-135, PS-136, PS-137, PS-138, PS-139, PS-140, PS-141, PS-142, PS-143, PS-144, PS-145, PS-146, PS-147, PS-148, PS-149, PS-150, PS-151, PS-152, PS-153, PS-154, PS-155, PS-156, PS-157, PS-158, PS-159, PS-160, PS-161, PS-162, PS-163, and PS-164 in Table 28.

In some embodiments, the product ion is selected as one from a group consisting of product ions identified in Table 29, including product ions falling within an identified m/z range of the m/z ratio identified in Table 29 and characterized as having a precursor ion having an m/z ratio within an identified m/z range of the m/z ratio identified in Table 29. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (+1.5). Thus, a composition may include a product ion having an m/z ratio that falls within at least one of the first range (±0.5), the second range (±0.8), or the third range (±1.0) of the product ion m/z ratio identified in Table 30, and characterized as having a precursor ion having an m/z ratio that falls within at least one of first range (±0.5), a second range (±1.0), or a third range (±1.0 of the precursor ion m/z ratio identified in Table 29.

Table 29 shows various parameters associated with the identification of the peptide and glycopeptides using LC and MRM-MS. The retention time (RT) represents the amount of time in minutes for the peptide elute from the chromatography column. The collision energy represents the energy applied to the peptide for creating fragments (i.e., product ions) such as, for example, in the 2^{nd} quadrupole of the triple quadrupole MS. The precursor m/z represents a ratio value associated with an ionized form having a precursor charge for the peptide or glycopeptide. The precursor ion is associated with a first product ion having a m/z ratio that was formed from a collision and with a second product ion having a m/z ratio that was formed from a collision.

TABLE 29
Mass Spectrometry-Related Characteristics for Sex-Associated Peptide Structures

| | Monoisotopic | | Collision | | | | 2nd |
|---|---|---|---|---|---|---|---|
| PS-ID | mass | RT | Energy- | Precursor | Precursor | Product | Product |
| NO. | (g/mol) | (min) | Volt | m/z | charge | m/z | m/z |
| 123 | 3141.28648 | 15.3 | 26 | 1048.1 | 3 | 366.1 | n/a |
| 124 | 3247.31309 | 16.9 | 30 | 1083.8 | 3 | 204.1 | n/a |
| 125 | 6062.47241 | 38.3 | 35 | 1214.1 | 5 | 274.1 | n/a |
| 126 | 3026.23708 | 16.2 | 25 | 1009.8 | 3 | 366.1 | n/a |
| 127 | 4242.66392 | 16.5 | 25 | 1062.2 | 4 | 366.1 | 1048.5 |
| 128 | 4498.00011 | 27.3 | 28 | 1125.6 | 4 | 366.1 | n/a |
| 129 | 5916.4145 | 38.3 | 30 | 1184.9 | 5 | 274.1 | n/a |
| 130 | 4060.8468 | 26.6 | 27 | 1016.2 | 4 | 366.1 | n/a |
| 131 | 6208.53032 | 38.3 | 35 | 1243.3 | 5 | 274.1 | n/a |
| 132 | 5406.24481 | 37.8 | 30 | 1082.6 | 5 | 274.1 | n/a |
| 133 | 4825.02159 | 30.7 | 36 | 1208 | 4 | 366.1 | n/a |
| 134 | 3578.4398 | 15.7 | 25 | 1193.8 | 3 | 366.1 | n/a |
| 135 | 5481.24918 | 31.6 | 20 | 1097.8 | 5 | 274.1 | n/a |
| 136 | 1638.782388 | 31.38 | 25 | 820.9 | 2 | 1052.5 | 678.3 |
| 137 | 3989.61269 | 30 | 20 | 998.9 | 4 | 366.1 | n/a |
| 138 | 3640.56461 | 35.7 | 20 | 911.4 | 4 | 274.1 | 820.4 |
| 139 | 4096.60602 | 16.7 | 20 | 1025.7 | 4 | 274.1 | 1048.5 |
| 140 | 4447.95094 | 23.9 | 15 | 891 | 5 | 366.1 | 913.5 |
| 141 | 4739.04635 | 25.3 | 18 | 949.2 | 5 | 274.1 | 913.5 |
| 142 | 3896.67588 | 37 | 20 | 975.7 | 4 | 366.1 | n/a |
| 143 | 4898.89152 | 17.1 | 30 | 1226.2 | 4 | 366.1 | 1048.5 |
| 144 | 4206.9047 | 26.4 | 26 | 1052.8 | 4 | 366.1 | n/a |
| 145 | 2147.169814 | 26.825 | 20 | 717.2 | 3 | 912 | 608.3 |
| 146 | 4752.83361 | 17.2 | 25 | 1189.7 | 4 | 274.1 | n/a |
| 147 | 5260.18691 | 37.9 | 30 | 1053.4 | 5 | 274.1 | n/a |
| 148 | 1435.79221 | 35.295 | 20 | 719.3 | 2 | 1160.7 | 903.5 |
| 149 | 2940.14866 | 4.1 | 27 | 981.4 | 3 | 366.1 | n/a |
| 150 | 3843.55478 | 30 | 20 | 962.4 | 4 | 366.1 | n/a |
| 151 | 3655.51799 | 27 | 35 | 1220.2 | 4 | 366.1 | 827.9 |
| 152 | 4014.81635 | 30.4 | 20 | 1004.7 | 4 | 366.1 | n/a |
| 153 | 4159.85386 | 31.8 | 25 | 1041.5 | 4 | 366.1 | n/a |
| 154 | 3868.75845 | 30.7 | 30 | 968.2 | 4 | 366.1 | n/a |
| 155 | 5107.17687 | 33.5 | 38 | 1277.8 | 4 | 366.1 | n/a |
| 156 | 4603.95931 | 37.2 | 30 | 922.2 | 5 | 204.1 | n/a |
| 157 | 5625.31909 | 37.7 | 30 | 1126.7 | 5 | 274.1 | 1302.1 |
| 158 | 4311.74559 | 27.2 | 38 | 1438.9 | 3 | 366.1 | 827.9 |
| 159 | 3287.34439 | 15.3 | 25 | 1096.8 | 3 | 366.1 | 1431.7 |
| 160 | 4187.77129 | 37.8 | 18 | 1048.4 | 4 | 1094 | n/a |
| 161 | 4450.94927 | 32.9 | 35 | 1114.2 | 4 | 204.1 | 1225.6 |
| 162 | 4461.7382 | 16.5 | 34 | 1117.2 | 4 | 366.1 | n/a |
| 163 | 3364.42258 | 26 | 25 | 1123.1 | 3 | 366.1 | 827.9 |
| 164 | 5627.30709 | 31.5 | 35 | 1408.6 | 4 | 274.1 | n/a |

Table 30A defines the peptide sequences for SEQ ID NOS: 183-196 from Table 28. Table 31 further identifies a corresponding protein SEQ TD NO. for each peptide sequence.

TABLE 30A
Peptide SEQ ID NOS

| (Pro- | | (Pep- |
|---|---|---|
| tein) | | tide) |
| SEQ | | SEQ |
| ID | | ID |
| NO. | Peptide.sequence | NO. |
| 175 | ENISDPTSPLR | 183 |
| 176 | LDVDQALNR | 184 |
| 177 | FNLTETSEAEIHQSFQHLLR | 185 |
| 178 | VLNFTTK | 186 |
| 179 | EHEGAIYPDNTTDFQR | 187 |
| 175 | IIVPLNNRENISDPTSPLR | 188 |
| 177 | FNLTETSEAEIHQSFQHLLR | 185 |
| 175 | IIVPLNNRENISDPTSPLR | 188 |
| 177 | FNLTETSEAEIHQSFQHLLR | 185 |
| 177 | FNLTETSEAEIHQSFQHLLR | 185 |
| 180 | HGIQYFNNNTQHSSLFMLNEVK | 189 |
| 175 | ENISDPTSPLR | 183 |
| 180 | HGIQYFNNNTQHSSLFMLNEVK | 189 |
| 179 | AGLQAFFQVQECNK | 190 |
| 179 | AGLQAFFQVQECNK | 190 |
| 181 | IYSGILNLSDITK | 191 |
| 179 | EHEGAIYPDNTTDFQR | 187 |
| 182 | VYIHPFHLVIHNESTCEQLAK | 192 |
| 182 | VYIHPFHLVIHNESTCEQLAK | 192 |
| 181 | IYPGVDFGGEELNVTFVK | 193 |
| 179 | EHEGAIYPDNTTDFQR | 187 |
| 175 | IIVPLNNRENISDPTSPLR | 188 |
| 175 | IIVPLNNRENISDPTSPLR | 188 |
| 179 | EHEGAIYPDNTTDFQR | 187 |
| 177 | FNLTETSEAEIHQSFQHLLR | 185 |
| 181 | IYSGILNLSDITK | 191 |
| 178 | LSSNSTK | 194 |
| 179 | AGLQAFFQVQECNK | 190 |
| 181 | LQAPLNYTEFQK | 195 |
| 181 | LQAPLNYTEFQKPICLPSK | 196 |
| 181 | LQAPLNYTEFQKPICLPSK | 196 |
| 181 | LQAPLNYTEFQKPICLPSK | 196 |
| 181 | LQAPLNYTEFQKPICLPSK | 196 |
| 177 | FNLTETSEAEIHQSFQHLLR | 185 |
| 177 | FNLTETSEAEIHQSFQHLLR | 185 |
| 181 | LQAPLNYTEFQK | 195 |
| 175 | ENISDPTSPLR | 183 |
| 181 | IYPGVDFGGEELNVTFVK | 193 |
| 181 | LQAPLNYTEFQKPICLPSK | 196 |
| 179 | EHEGAIYPDNTTDFQR | 187 |
| 181 | LQAPLNYTEFQK | 195 |
| 180 | HGIQYFNNNTQHSSLFMLNEVK | 189 |

[11] Table 30B provides an indication of particular markers and includes the starting position of the peptide sequence within the protein sequence and the end position of the peptide sequence within the protein sequence.

TABLE 30B
Markers and Protein Positions

| PS- | | | Start | End |
|---|---|---|---|---|
| ID | | Peptide. | posi- | posi- |
| NO. | PS-NAME | sequence | tion | tion |
| 123 | IGJ | ENISDPTSP | 70 | 80 |
| | (71)- | LR | | |
| | 5401 | | | |
| 124 | SHBG | LDVDQALNR | 373 | 381 |
| | (380)- | | | |
| | 5402 | | | |
| 125 | AACT | FNLTETSEA | 105 | 124 |
| | (106)- | EIHQSFQHL | | |
| | 7614 | LR | | |
| 126 | CO6 | VLNFTTK | 322 | 328 |
| | (324)- | | | |
| | 5402 | | | |
| 127 | CERU | EHEGAIYPD | 129 | 144 |
| | (138)- | NTTDFQR | | |
| | 5412 | | | |
| 128 | IGJ | IIVPLNNRE | 62 | 80 |
| | (71MC)- | NISDPTSPL | | |
| | 5412 | R | | |
| 129 | AACT | FNLTETSEA | 105 | 124 |
| | (106)- | EIHQSFQHL | | |
| | 7604 | LR | | |
| 130 | IGJ | IIVPLNNRE | 62 | 80 |
| | (71MC)- | NISDPTSPL | | |
| | 5401 | R | | |
| 131 | AACT | FNLTETSEA | 105 | 124 |
| | (106)- | EIHQSFQHL | | |
| | 7624 | LR | | |
| 132 | AACT | FNLTETSEA | 105 | 124 |
| | (106)- | EIHQSFQHL | | |
| | 6513 | LR | | |
| 133 | KNG1 | HGIQYFNNNT | 162 | 183 |
| | (169)- | QHSSLFMLNE | | |
| | 5402 | VK | | |
| 134 | IGJ | ENISDPTSPL | 70 | 80 |
| | (71)- | R | | |
| | 5412 | | | |
| 135 | KNG1 | HGIQYFNNNT | 162 | 183 |
| | (169)- | QHSSLFMLNE | | |
| | 6503 | VK | | |
| 136 | CERU | AGLQAFFQVQ | 346 | 359 |
| | (358)- | ECNK | | |
| | NON- | | | |
| | GLYCOS. | | | |
| 137 | CERU | AGLQAFFQVQ | 346 | 359 |
| | (358)- | ECNK | | |
| | 5412 | | | |
| 138 | KLKB1 | IYSGILNLSD | 447 | 459 |
| | (453)- | ITK | | |
| | 5402 | | | |
| 139 | CERU | EHEGAIYPDN | 129 | 144 |
| | (138)- | TTDFQR | | |
| | 5402 | | | |
| 140 | ANGT | VYIHPFHLVI | 36 | 56 |
| | (47)- | HNESTCEQLA | | |
| | 5401 | K | | |
| 141 | ANGT | VYIHPFHLVI | 36 | 56 |
| | (47)- | HNESTCEQLA | | |
| | 5402 | K | | |
| 142 | KLKB1 | IYPGVDFGGE | 296 | 313 |
| | (308)- | ELNVTFVK | | |
| | 5401 | | | |
| 143 | CERU | EHEGAIYPDN | 129 | 144 |
| | (138)- | TTDFQR | | |
| | 6513 | | | |
| 144 | IGJ | IIVPLNNREN | 62 | 80 |
| | (71MC)- | ISDPTSPLR | | |
| | 5411 | | | |
| 145 | IGJ | IIVPLNNREN | 61 | 80 |
| | (71MC)- | ISDPTSPLR | | |
| | NON- | | | |
| | GLYCOS. | | | |
| 146 | CERU | EHEGAIYPDN | 129 | 144 |
| | (138)- | TTDFQR | | |
| | 6503 | | | |
| 147 | AACT | FNLTETSEAE | 105 | 124 |
| | (106)- | IHQSFQHLLR | | |
| | 6503 | | | |
| 148 | KLKB1 | IYSGILNLSD | 447 | 459 |
| | (453)- | ITK | | |
| | NON- | | | |
| | GLYCOS. | | | |
| 149 | CO6 | LSSNSTK | 852 | 858 |
| | (855)- | | | |
| | 5402 | | | |
| 150 | CERU | AGLQAFFQVQ | 346 | 359 |
| | (358)- | ECNK | | |
| | 5402 | | | |
| 151 | KLKB1 | LQAPLNYTEF | 489 | 500 |
| | (494MC)- | QK | | |
| | 5402 | | | |
| 152 | KLKB1 | LQAPLNYTEF | 489 | 507 |
| | (494)- | QKPICLPSK | | |
| | 5410 | | | |
| 153 | KLKB1 | LQAPLNYTEF | 489 | 507 |
| | (494)- | QKPICLPSK | | |
| | 5401 | | | |
| 154 | KLKB1 | LQAPLNYTEF | 489 | 507 |
| | (494)- | QKPICLPSK | | |
| | 5400 | | | |
| 155 | KLKB1 | LQAPLNYTEF | 489 | 507 |
| | (494)- | QKPICLPSK | | |
| | 6503 | | | |
| 156 | AACT | FNLTETSEAE | 105 | 124 |
| | (106)- | IHQSFQHLLR | | |
| | 5402 | | | |
| 157 | AACT | FNLTETSEAE | 105 | 124 |
| | (106)- | IHQSFQHLLR | | |
| | 7603 | | | |
| 158 | KLKB1 | LQAPLNYTEF | 489 | 500 |
| | (494MC)- | QK | | |
| | 6503 | | | |
| 159 | IGJ | ENISDPTSPL | 70 | 80 |
| | (71)- | R | | |
| | 5411 | | | |
| 160 | KLKB1 | IYPGVDFGGE | 296 | 313 |
| | (308)- | ELNVTFVK | | |
| | 5402 | | | |
| 161 | KLKB1 | LQAPLNYTEF | 489 | 507 |
| | (494)- | QKPICLPSK | | |
| | 5402 | | | |
| 162 | CERU | EHEGAIYPDN | 129 | 144 |
| | (138)- | TTDFQR | | |
| | 6502 | | | |
| 163 | KLKB1 | LQAPLNYTEF | 489 | 500 |
| | (494MC)- | QK | | |
| | 5401 | | | |
| 164 | KNG1 | HGIQYFNNNT | 162 | 183 |
| | (169)- | QHSSLFMLNE | | |
| | 6513 | VK | | |

Table 31 identifies the proteins of SEQ TD NOS: 175-182 from Table 28. Table 31 identifies a corresponding protein abbreviation, protein name, and amino acid sequence for each of protein SEQ TD NOS: 175-182. Further, Table 31 identifies a corresponding Uniprot ID for each of protein SEQ TD NOS: 175-182.

TABLE 31
Protein SEQ ID NOS

| | | | Prot | |
|---|---|---|---|---|
| Pro- | Pro- | Uni- | SEQ | |
| tein | tein | prot | ID | |
| Abbrev | Name | ID | NO. | Protein Sequence |
| IGJ | Immuno- | P01591 | 175 | MKNHLLFWGVLAVFIKA |
| | globulin | | | VHVKAQEDERIVLVDNK |
| | J chain | | | CKCARITSRIIRSSEDP |
| | | | | NEDIVERNIRIIVPLNN |
| | | | | RENISDPTSPLRTRFVY |
| | | | | HLSDLCKKCDPTEVELD |
| | | | | NQIVTATQSNICDEDSA |
| | | | | TETCYTYDRNKCYTAVV |
| | | | | PLVYGGETKMVETALTP |
| | | | | DACYPD |
| SHBG | Sex | P04278 | 176 | MESRGPLATSRLLLLLL |
| | hormone- | | | LLLLRHTRQGWALRPVL |
| | binding | | | PTQSAHDPPAVHLSNGP |
| | globulin | | | GQEPIAVMTFDLTKITK |
| | | | | TSSSFEVRTWDPEGVIF |
| | | | | YGDTNPKDDWFMLGLRD |
| | | | | GRPEIQLHNHWAQLTVG |
| | | | | AGPRLDDGRWHQVEVKM |
| | | | | EGDSVLLEVDGEEVLRL |
| | | | | RQVSGPLTSKRHPIMRI |
| | | | | ALGGLLFPASNLRLPLV |
| | | | | PALDGCLRRDSWLDKQA |
| | | | | EISASAPTSLRSCDVES |
| | | | | NPGIFLPPGTQAEFNLR |
| | | | | DIPQPHAEPWAFSLDLG |
| | | | | LKQAAGSGHLLALGTPE |
| | | | | NPSWLSLHLQDQKVVLS |
| | | | | SGSGPGLDLPLVLGLPL |
| | | | | QLKLSMSRVVLSQGSKM |
| | | | | KALALPPLGLAPLLNLW |
| | | | | AKPQGRLFLGALPGEDS |
| | | | | STSFCLNGLWAQGQRLD |
| | | | | VDQALNRSHEIWTHSCP |
| | | | | QSPGNGTDASH |
| AACT | Alpha- | P01011 | 177 | MERMLPLLALGLLAAGF |
| | 1-anti- | | | CPAVLCHPNSPLDEENL |
| | chymo- | | | TQENQDRGTHVDLGLAS |
| | trypsin | | | ANVDFAFSLYKQLVLKA |
| | | | | PDKNVIFSPLSISTALA |
| | | | | FLSLGAHNTTLTEILKG |
| | | | | LKFNLTETSEAEIHQSF |
| | | | | QHLLRTLNQSSDELQLS |
| | | | | MGNAMFVKEQLSLLDRF |
| | | | | TEDAKRLYGSEAFATDF |
| | | | | QDSAAAKKLINDYVKNG |
| | | | | TRGKITDLIKDLDSQTM |
| | | | | MVLVNYIFFKAKWEMPF |
| | | | | DPQDTHQSRFYLSKKKW |
| | | | | VMVPMMSLHHLTIPYFR |
| | | | | DEELSCTVVELKYTGNA |
| | | | | SALFILPDQDKMEEVEA |
| | | | | MLLPETLKRWRDSLEFR |
| | | | | EIGELYLPKFSISRDYN |
| | | | | LNDILLQLGIEEAFTSK |
| | | | | ADLSGITGARNLAVSQV |
| | | | | VHKAVLDVFEEGTEASA |
| | | | | ATAVKITLLSALVETRT |
| | | | | IVRFNRPFLMIIVPTDT |
| | | | | QNIFFMSKVTNPKQA |
| CO6 | Comple- | P13671 | 178 | MARRSVLYFILLNALIN |
| | ment | | | KGQACFCDHYAWTQWTS |
| | compo- | | | CSKTCNSGTQSRHRQIV |
| | nent C6 | | | VDKYYQENFCEQICSKQ |
| | | | | ETRECNWQRCPINCLLG |
| | | | | DFGPWSDCDPCIEKQSK |
| | | | | VRSVLRPSQFGGQPCTA |
| | | | | PLVAFQPCIPSKLCKIE |
| | | | | EADCKNKFRCDSGRCIA |
| | | | | RKLECNGENDCGDNSDE |
| | | | | RDCGRTKAVCTRKYNPI |
| | | | | PSVQLMGNGFHFLAGEP |
| | | | | RGEVLDNSFTGGICKTV |
| | | | | KSSRTSNPYRVPANLEN |
| | | | | VGFEVQTAEDDLKTDFY |
| | | | | KDLTSLGHNENQQGSFS |
| | | | | SQGGSSFSVPIFYSSKR |
| | | | | SENINHNSAFKQAIQAS |
| | | | | HKKDSSFIRIHKVMKVL |
| | | | | NFTTKAKDLHLSDVFLK |
| | | | | ALNHLPLEYNSALYSRI |
| | | | | FDDFGTHYFTSGSLGGV |
| | | | | YDLLYQFSSEELKNSGL |
| | | | | TEEEAKHCVRIETKKRV |
| | | | | LFAKKTKVEHRCTTNKL |
| | | | | SEKHEGSFIQGAEKSIS |
| | | | | LIRGGRSEYGAALAWEK |
| | | | | GSSGLEEKTFSEWLESV |
| | | | | KENPAVIDFELAPIVDL |
| | | | | VRNIPCAVTKRNNLRKA |
| | | | | LQEYAAKFDPCQCAPCP |
| | | | | NNGRPTLSGTECLCVCQ |
| | | | | SGTYGENCEKQSPDYKS |
| | | | | NAVDGQWGCWSSWSTCD |
| | | | | ATYKRSRTRECNNPAPQ |
| | | | | RGGKRCEGEKRQEEDCT |
| | | | | FSIMENNGQPCINDDEE |
| | | | | MKEVDLPEIEADSGCPQ |
| | | | | PVPPENGFIRNEKQLYL |
| | | | | VGEDVEISCLTGFETVG |
| | | | | YQYFRCLPDGTWRQGDV |
| | | | | ECQRTECIKPVVQEVLT |
| | | | | ITPFORLYRIGESIELT |
| | | | | CPKGFVVAGPSRYTCQG |
| | | | | NSWTPPISNSLTCEKDT |
| | | | | LTKLKGHCQLGQKQSGS |
| | | | | ECICMSPEEDCSHHSED |
| | | | | LCVFDTDSNDYFTSPAC |
| | | | | KFLAEKCLNNQQLHFLH |
| | | | | IGSCQDGRQLEWGLERT |
| | | | | RLSSNSTKKESCGYDTC |
| | | | | YDWEKCSASTSKCVCLL |
| | | | | PPQCFKGGNQLYCVKMG |
| | | | | SSTSEKTLNICEVGTIR |
| | | | | CANRKMEILHPGKCLA |
| CERU | Cerulo- | P00450 | 179 | MKILILGIFLFLCSTPA |
| | plasmin | | | WAKEKHYYIGIIETTWD |
| | | | | YASDHGEKKLISVDTEH |
| | | | | SNIYLQNGPDRIGRLYK |
| | | | | KALYLQYTDETFRTTIE |
| | | | | KPVWLGFLGPIIKAETG |
| | | | | DKVYVHLKNLASRPYTF |
| | | | | HSHGITYYKEHEGAIYP |
| | | | | DNTTDFQRADDKVYPGE |
| | | | | QYTYMLLATEEQSPGEG |
| | | | | DGNCVTRIYHSHIDAPK |
| | | | | DIASGLIGPLIICKKDS |
| | | | | LDKEKEKHIDREFVVMF |
| | | | | SVVDENFSWYLEDNIKT |
| | | | | YCSEPEKVDKDNEDFQE |
| | | | | SNRMYSVNGYTFGSLPG |
| | | | | LSMCAEDRVKWYLFGMG |
| | | | | NEVDVHAAFFHGQALTN |
| | | | | KNYRIDTINLFPATLFD |
| | | | | AYMVAQNPGEWMLSCQN |
| | | | | LNHLKAGLQAFFQVQEC |
| | | | | NKSSSKDNIRGKHVRHY |
| | | | | YIAAEEIIWNYAPSGID |
| | | | | IFTKENLTAPGSDSAVF |
| | | | | FEQGTTRIGGSYKKLVY |
| | | | | REYTDASFTNRKERGPE |
| | | | | EEHLGILGPVIWAEVGD |
| | | | | TIRVTFHNKGAYPLSIE |
| | | | | PIGVRFNKNNEGTYYSP |
| | | | | NYNPQSRSVPPSASHVA |
| | | | | PTETFTYEWTVPKEVGP |
| | | | | TNADPVCLAKMYYSAVD |
| | | | | PTKDIFTGLIGPMKICK |
| | | | | KGSLHANGRQKDVDKEF |
| | | | | YLFPTVFDENESLLLED |
| | | | | NIRMFTTAPDQVDKEDE |
| | | | | DFQESNKMHSMNGFMYG |
| | | | | NQPGLTMCKGDSVVWYL |
| | | | | FSAGNEADVHGIYFSGN |
| | | | | TYLWRGERRDTANLFPQ |
| | | | | TSLTLHMWPDTEGTFNV |
| | | | | ECLTTDHYTGGMKQKYT |
| | | | | VNQCRRQSEDSTFYLGE |
| | | | | RTYYIAAVEVEWDYSPQ |
| | | | | REWEKELHHLQEQNVSN |
| | | | | AFLDKGEFYIGSKYKKV |
| | | | | VYRQYTDSTFRVPVERK |
| | | | | AEEEHLGILGPQLHADV |
| | | | | GDKVKIIFKNMATRPYS |
| | | | | IHAHGVQTESSTVTPTL |
| | | | | PGETLTYVWKIPERSGA |
| | | | | GTEDSACIPWAYYSTVD |
| | | | | QVKDLYSGLIGPLIVCR |
| | | | | RPYLKVFNPRRKLEFAL |
| | | | | LFLVFDENESWYLDDNI |
| | | | | KTYSDHPEKVNKDDEEF |
| | | | | IESNKMHAINGRMFGNL |
| | | | | QGLTMHVGDEVNWYLMG |
| | | | | MGNEIDLHTVHFHGHSF |
| | | | | QYKHRGVYSSDVFDIFP |
| | | | | GTYQTLEMFPRTPGIWL |
| | | | | LHCHVTDHIHAGMETTY |
| | | | | TVLQNEDTKSG |
| KNG1 | Kinino- | P01042 | 180 | MKLITILFLCSRLLLSL |
| | gen-1 | | | TQESQSEEIDCNDKDLF |
| | | | | KAVDAALKKYNSQNQSN |
| | | | | NQFVLYRITEATKTVGS |
| | | | | DTFYSFKYEIKEGDCPV |
| | | | | QSGKTWQDCEYKDAAKA |
| | | | | ATGECTATVGKRSSTKF |
| | | | | SVATQTCQITPAEGPVV |
| | | | | TAQYDCLGCVHPISTQS |
| | | | | PDLEPILRHGIQYFNNN |
| | | | | TQHSSLFMLNEVKRAQR |
| | | | | QVVAGLNFRITYSIVQT |
| | | | | NCSKENFLFLTPDCKSL |
| | | | | WNGDTGECTDNAYIDIQ |
| | | | | LRIASFSQNCDIYPGKD |
| | | | | FVQPPTKICVGCPRDIP |
| | | | | TNSPELEETLTHTITKL |
| | | | | NAENNATFYFKIDNVKK |
| | | | | ARVQVVAGKKYFIDFVA |
| | | | | RETTCSKESNEELTESC |
| | | | | ETKKLGQSLDCNAEVYV |
| | | | | VPWEKKIYPTVNCQPLG |
| | | | | MISLMKRPPGFSPFRSS |
| | | | | RIGEIKEETTVSPPHTS |
| | | | | MAPAQDEERDSGKEQGH |
| | | | | TRRHDWGHEKQRKHNLG |
| | | | | HGHKHERDQGHGHQRGH |
| | | | | GLGHGHEQQHGLGHGHK |
| | | | | FKLDDDLEHQGGHVLDH |
| | | | | GHKHKHGHGHGKHKNKG |
| | | | | KKNGKHNGWKTEHLASS |
| | | | | SEDSTTPSAQTQEKTEG |
| | | | | PTPIPSLAKPGVTVTFS |
| | | | | DFQDSDLIATMMPPISP |
| | | | | APIQSDDDWIPDIQIDP |
| | | | | NGLSFNPISDFPDTTSP |
| | | | | KCPGRPWKSVSEINPTT |
| | | | | QMKESYYFDLTDGLS |
| KLKB1 | Plasma | P03952 | 181 | MILFKQATYFISLFATV |
| | Kalli- | | | SCGCLTQLYENAFFRGG |
| | krein | | | DVASMYTPNAQYCQMRC |
| | | | | TFHPRCLLFSFLPASSI |
| | | | | NDMEKRFGCFLKDSVTG |
| | | | | TLPKVHRTGAVSGHSLK |
| | | | | QCGHQISACHRDIYKGV |
| | | | | DMRGVNFNVSKVSSVEE |
| | | | | CQKRCTNNIRCQFFSYA |
| | | | | TQTFHKAEYRNNCLLKY |
| | | | | SPGGTPTAIKVLSNVES |
| | | | | GFSLKPCALSEIGCHMN |
| | | | | IFQHLAFSDVDVARVLT |
| | | | | PDAFVCRTICTYHPNCL |
| | | | | FFTFYTNVWKIESQRNV |
| | | | | CLLKTSESGTPSSSTPQ |
| | | | | ENTISGYSLLTCKRTLP |
| | | | | EPCHSKIYPGVDFGGEE |
| | | | | LNVTFVKGVNVCQETCT |
| | | | | KMIRCQFFTYSLLPEDC |
| | | | | KEEKCKCFLRLSMDGSP |
| | | | | TRIAYGTQGSSGYSLRL |
| | | | | CNTGDNSVCTTKTSTRI |
| | | | | VGGTNSSWGEWPWQVSL |
| | | | | QVKLTAQRHLCGGSLIG |
| | | | | HQWVLTAAHCFDGLPLQ |
| | | | | DVWRIYSGILNLSDITK |
| | | | | DTPFSQIKEIIIHQNYK |
| | | | | VSEGNHDIALIKLQAPL |
| | | | | NYTEFQKPICLPSKGDT |
| | | | | STIYTNCWVTGWGFSKE |
| | | | | KGEIQNILQKVNIPLVT |
| | | | | NEECQKRYQDYKITQRM |
| | | | | VCAGYKEGGKDACKGDS |
| | | | | GGPLVCKHNGMWRLVGI |
| | | | | TSWGEGCARREQPGVYT |
| | | | | KVAEYMDWILEKTQSSD |
| | | | | GKAQMQSPA |
| ANGT | Angio- | P01019 | 182 | MRKRAPQSEMAPAGVSL |
| | tensin- | | | RATILCLLAWAGLAAGD |
| | ogen | | | RVYIHPFHLVIHNESTC |
| | | | | EQLAKANAGKPKDPTFI |
| | | | | PAPIQAKTSPVDEKALQ |
| | | | | DQLVLVAAKLDTEDKLR |
| | | | | AAMVGMLANFLGFRIYG |
| | | | | MHSELWGVVHGATVLSP |
| | | | | TAVFGTLASLYLGALDH |
| | | | | TADRLQAILGVPWKDKN |
| | | | | CTSRLDAHKVLSALQAV |
| | | | | QGLLVAQGRADSQAQLL |
| | | | | LSTVVGVFTAPGLHLKQ |
| | | | | PFVQGLALYTPVVLPRS |
| | | | | LDFTELDVAAEKIDRFM |
| | | | | QAVTGWKTGCSLMGASV |
| | | | | DSTLAFNTYVHFQGKMK |
| | | | | GFSLLAEPQEFWVDNST |
| | | | | SVSVPMLSGMGTFQHWS |
| | | | | DIQDNFSVTQVPFTESA |
| | | | | CLLLIQPHYASDLDKVE |
| | | | | GLTFQQNSLNWMKKLSP |
| | | | | RTIHLTMPQLVLQGSYD |
| | | | | LQDLLAQAELPAILHTE |
| | | | | LNLQKLSNDRIRVGEVL |
| | | | | NSIFFELEADEREPTES |
| | | | | TQQLNKPEVLEVTLNRP |
| | | | | FLFAVYDQSATALHFLG |
| | | | | RVANPLSTA |

Table 32 identifies and defines the glycan structures included in Table 28, all of which are N-glycans. Table 32 identifies a coded representation of the composition for each glycan structure included in Table 28. As used herein, the 4-digit GL NO. is a designation that represents the number of hexoses, the number of HexNAcs, the number of Fucoses, and the number of Neuraminic Acids. Table 32 illustrates the symbol structure and composition of detected glycan moieties that correspond to glycopeptides of Table 28 based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. For reference, N-linked glycans have a glycan attached to the amino acid asparagine and O-linked glycans have a glycan attached to either a serine or a threonine.

[14] The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.
[15] Referring back to Table 32, for some entries, there are two symbol structures provided for one Glycan Structure GL NO such as, for example, Glycan Structure GL NO 5400. Thus, the identify of a peptide that references a Glycan Structure GL NO that has two symbol structures could be one of two possibilities based on the MRM of the LC-MS analysis. In some instances, a bracket symbol is used as part of the Symbol Structure to indicate that the precise bonding linkage is not exactly known, but that the linking line segment is attached to one of the plurality of adjacent carbohydrates immediately adjacent to the bracket.

TABLE 32
Glycan structure GL NO, symbol structure, and composition

| Glycan | | | |
|---|---|---|---|
| Structure | Symbol | Glycan | Glycan_{—} |
| GL NO. | Structure. | Composition | MW |
| 5402 | [symbol] | Hex(5)HexNAc(4)Fuc(0)NeuAc(2) | 2204.772392 |
| 6503 | [symbol] | Hex(6)HexNAc(5)Fuc(0)NeuAc(3) | 2860.99999 |
| 6513 | [symbol] | Hex(6)HexNAc(5)Fuc(1)NeuAc(3) | 3007.057896 |
| 7603 | [symbol] | Hex(7)HexNAc(6)Fuc(0)NeuAc(3) | 3226.132178 |
| 7604 | [symbol] | Hex(7)HexNAc(6)Fuc(0)NeuAc(4) | 3517.227588 |
| 7614 | [symbol] | Hex(7)HexNAc(6)Fuc(1)NeuAc(4) | 3663.285494 |
| 7624 | [symbol] | Hex(7)HexNAc(6)Fuc(2)NeuAc(4) | 3809.3434 |
| 5401 | [symbol] | Hex(5)HexNAc(4)Fuc(0)NeuAc(1) | 1913.676982 |
| 5412 | [symbol] | Hex(5)HexNAc(4)Fuc(1)NeuAc(2) | 2350.830298 |
| 6502 | [symbol] | Hex(6)HexNAc(5)Fuc(0)NeuAc(2) | 2569.90458 |
| 5411 | [symbol] | Hex(5)HexNAc(4)Fuc(1)NeuAc(1) | 2059.734888 |
| 5400 | [symbol] | Hex(5)HexNAc(4)Fuc(0)NeuAc(0) | 1622.581572 |
| 5410 | [symbol] | Hex(5)HexNAc(4)Fuc(1)NeuAc(0) | 1768.639478 |
| 5502 | [symbol] | Hex(5)HexNAc(5)Fuc(0)NeuAc(2) | 2407.85176 |

Legend for Table 32:
● Glc [symbol]
Gal [symbol]
Man [symbol]
Fuc [symbol]
Neu5Ac
▪ GlcNAc [symbol]
GalNAc [symbol]
ManNAc

[16] The identity of the various monosaccharides is illustrated by the Legend section located at the end of Table 32. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.
[17] Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.
[18] The peptide structures and the transitions produced therefrom, as described herein, may be useful as quality control compositions and data with respect to a group of samples. A transition includes a precursor ion and at least one product ion grouping. As reviewed herein, the peptide structures in Table 28, as well as their corresponding precursor ion and product ion groupings (these ions having defined m/z ratios or m/z ratios that fall within the m/z ranges identified herein) in Table 29, can be used in mass spectrometry-based analyses to diagnose and facilitate treatment of diseases, such as, for example, adenoma or CRC.
[19] Aspects of the disclosure include methods for analyzing one or more peptide structures, as described herein. In some embodiments, the methods involve processing a sample from a subject, such as a patient, to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.
[20] In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 29 and the m/z ratio is within a predetermined interval. In some embodiments, the methods involve generating quantification (e.g., abundance) data for the one or more product ions detected using the reaction monitoring mass spectrometry system.
[21] In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### XIV.D. Representative Experimental Results

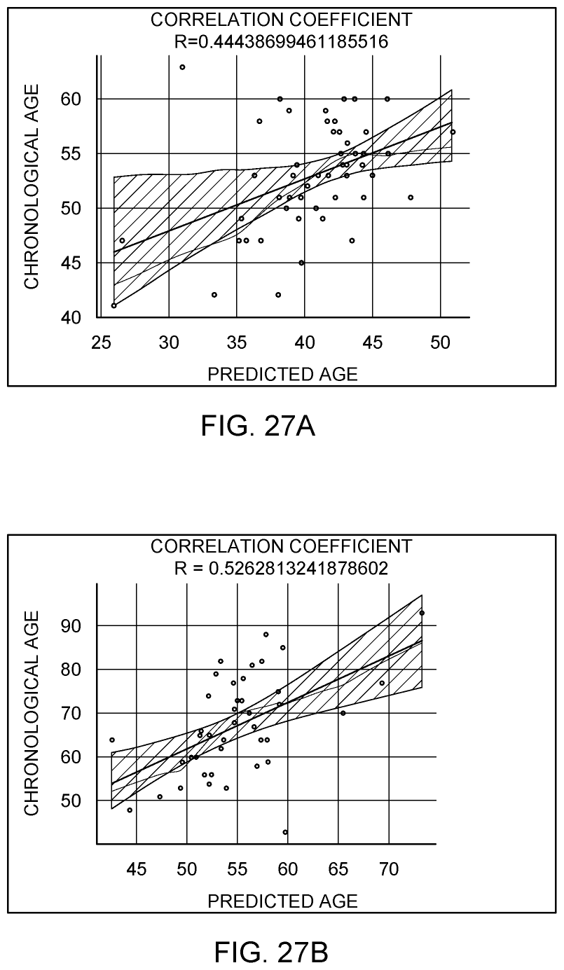

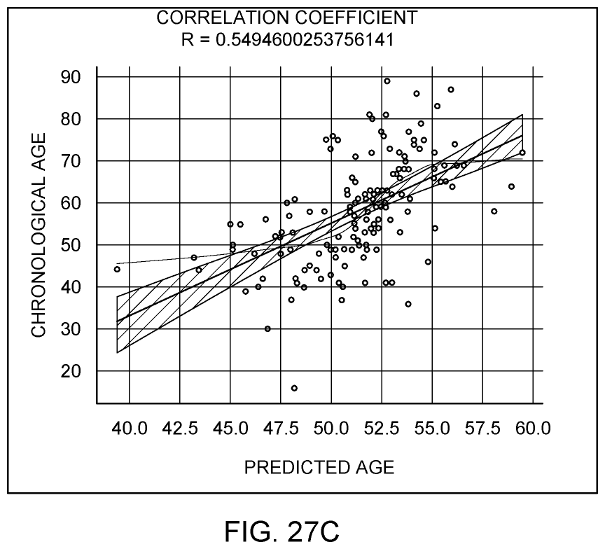

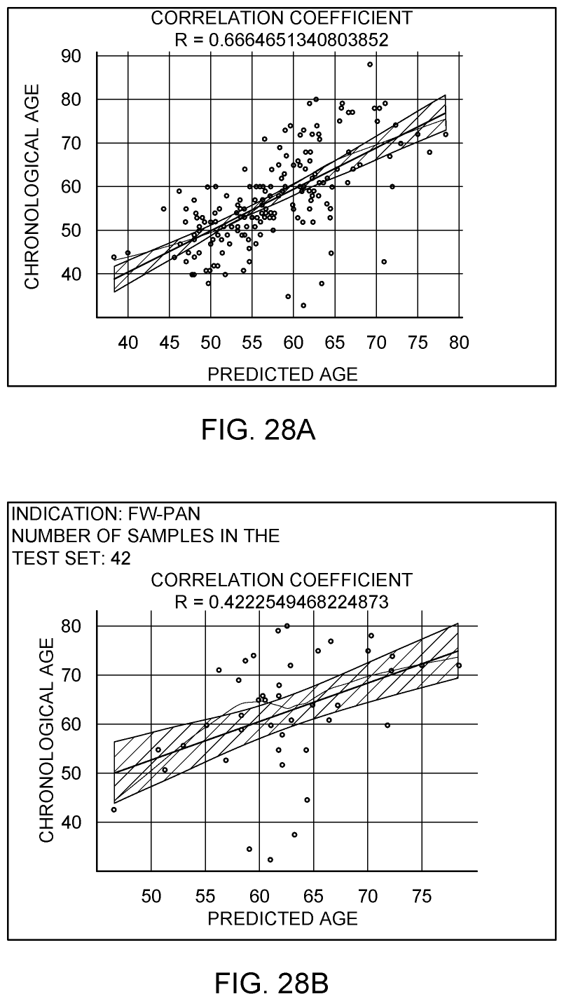

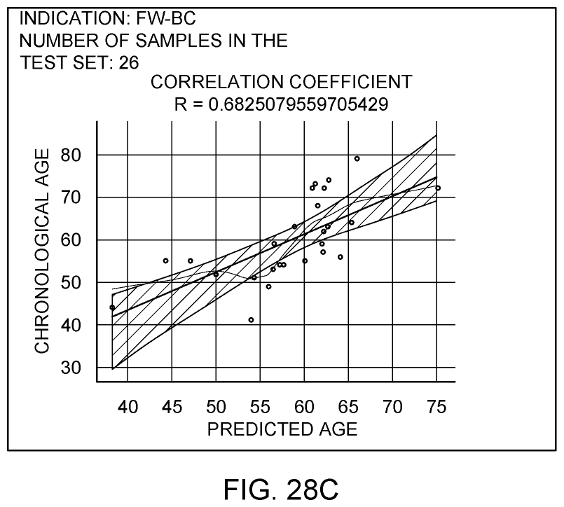

To assess the performance of the model for age-associated peptide structures (biomarkers), results are illustrated in FIGS. 27A-27C for healthy cohort of samples, and in FIGS. 28A-28C for samples from diseased subjects and samples from healthy subjects. It is worthwhile to note that in FIGS. 27A-27C and 28A-28C that the correlation coefficient R was greater than 0.2 suggesting that the correlation between the chronological age and the predicted age were sufficiently accurate indicating an absence of a quality control issue with respect to this test.

FIGS. 27A, 27B, and 27C illustrate plots of predicted age versus chronological age for healthy subjects to determine correlation coefficients of various cohorts.

FIGS. 28A, 28B, and 28C illustrate plots of predicted age versus chronological age for samples that include both disease and healthy subjects to determine correlation coefficients of various cohorts.

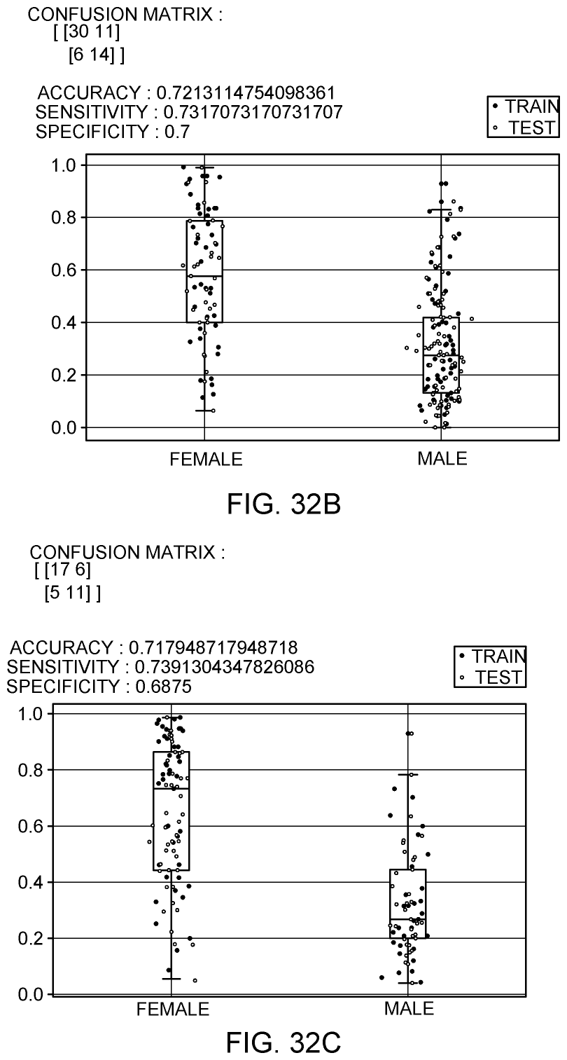

To assess the performance of the model for sex-associated peptide structures (biomarkers), results are illustrated in FIGS. 32A-32C for samples with both disease and healthy conditions.

FIGS. 32A, 32B, and 32C illustrate plots of accuracy score, sensitivity score, and specificity score for training and test datasets.

### Exemplary Embodiments for Quality Control

Methods of the disclosure encompass various embodiments of quality control glycopeptide compositions that can be utilized as an indicator for whether or not the method had one or more errors of any kind. In various embodiments, a grouping of glycopeptide and peptide compositions are associated with a ground truth about an expected age or range of ages of members of a group of subjects having samples tested therefor. The age information that is known to be real or true may be based on prior knowledge from annotation of the respective samples, for example.

In specific embodiments, a batch of samples in need of analysis or measuring, such as for disease diagnosis and/or treatment, are analyzed for the disease diagnosis and/or treatment. The samples are also analyzed or measured for one or more peptide structures of Table 23, and the outcome of this quality check analysis or measuring is used as an indicator for the quality of the method for disease diagnosis and/or treatment. If the one or more peptide structures of Table 23 are not present in the samples as predicted (qualitatively or quantitatively), this indicates that collectively the predicted age of the subjects is erroneous, and it can be considered that there was an error in the process and/or an error with sample annotation (including sample annotation for one or more of the samples, in some cases at least the majority of samples). Examples of points of error in the method include measuring by mass spectrometry, ionizing glycopeptides with a mass spectrometer, digesting proteins into glycopeptides, annotating one or more samples, user error, equipment failure, inputting data associated with each of the subjects into a database that includes chronological age, randomizing samples and mapping the samples to an autosampler for inputting into a LCMS, or a combination thereof.

In various embodiments, there are methods of performing quality control for a group of subject samples, comprising the step of assaying or measuring for one or more age-related peptide structures identified in Table 23. In some cases, there is comparison of one or more age-related peptide structures assayed for or measured from the samples to a reference set of the corresponding one or more age-related peptide structures in Table 23. In embodiments in which the one or more age-related peptide structures are identified in the samples as predicted, the method is considered to have suitable quality, the method is considered to lack any substantive errors, and so forth. In embodiments wherein the one or more age-related peptide structures are not identified in the samples or are at an incorrect quantity (or range of quantity), including one that is not predicted, the method can be considered not to have suitable quality, the method can be considered to have one or more substantive processing, instrumental, and/or human errors, and so forth.

In particular embodiments, the methods allow utilization of one or more age-related peptide structures in Table 23 as an indicator of quality control for a batch of samples, and the presence or quantity of the structures are indicative of a specific age (within a year) of a subject or of a range of age of a subject. The range of age may be 2, 3, 4, 5, 6, 7, 8, 9, 10, or more years. The range may be within 2, 3, 4, 5, 6, 7, 8, 9, or 10 or more years of the chronological age. The range may be, or may be about, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 years within the chronological age.

Methods of the disclosure encompass various embodiments of quality control glycopeptide compositions that can be utilized as an indicator for whether or not the method had one or more errors of any kind. In various embodiments, a grouping of glycopeptide and peptide compositions are associated with a ground truth about an expected biological sex of members of a group of subjects having samples tested therefor. The sex information that is known to be real or true may be based on prior knowledge from annotation of the respective samples, for example.

In specific embodiments, a batch of samples in need of analysis or measuring, such as for disease diagnosis and/or treatment, are analyzed for the disease diagnosis and/or treatment. The samples are also analyzed or measured for one or more peptide structures of Table 28, and the outcome of this quality check analysis or measuring is used as an indicator for the quality of the method for disease diagnosis and/or treatment. If the one or more peptide structures of Table 28 are not present in the samples as predicted (qualitatively or quantitatively), this indicates that collectively the predicted sex of the subjects is erroneous, and it can be considered that there was an error in the process and/or an error with sample annotation (including sample annotation for one or more of the samples, in some cases at least the majority of samples). Examples of points of error in the method include measuring by mass spectrometry, ionizing glycopeptides with a mass spectrometer, digesting proteins into glycopeptides, annotating one or more samples, user error, equipment failure, inputting data associated with each of the subjects into a database that includes annotated sex, randomizing samples and mapping the samples to an autosampler for inputting into a LCMS, and a combination thereof.

In various embodiments, there are methods of performing quality control for a group of subject samples, comprising the step of assaying or measuring for one or more sex-related peptide structures identified in Table 28. In some cases, there is comparison of one or more sex-related peptide structures assayed for or measured from the samples to a reference set of the corresponding one or more sex-related peptide structures in Table 28. In embodiments in which the one or more sex-related peptide structures are identified in the samples as predicted, the method is considered to have suitable quality, the method is considered to lack any substantive errors, and so forth. In embodiments wherein the one or more sex-related peptide structures are not identified in the samples or are at an incorrect quantity (or range of quantity), including one that is not predicted, the method can be considered not to have suitable quality, the method can be considered to have one or more substantive processing, instrumental, and/or human errors, and so forth.

### I. Representative Experimental Results—Ovarian Cancer

### I.A. Exemplary Retrospective Analysis

### I.A.1. Sample Acquisition

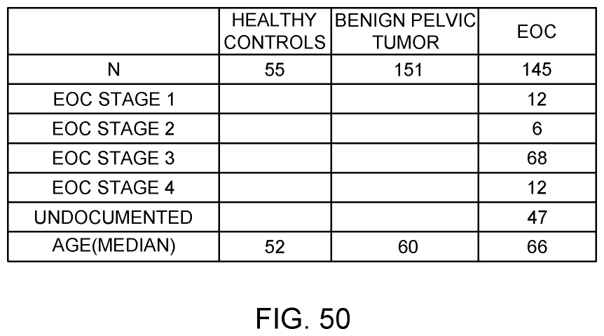

FIG. 50 is a table describing the distribution of the samples acquired in this exemplary retrospective analysis in accordance with one or more embodiments. As shown in FIG. 50, serum samples were acquired from a commercial biobank for 151 women with benign pelvic masses, 145 women with malignant epithelial ovarian cancer (EOC), and 55 healthy controls. Information on stage of EOC was available in 98 of the 145 patients with EOC. All samples were obtained prior to therapeutic intervention. Information on the benign or malignant nature of tumors was based on histopathological analysis of tissue specimens.

### I.A.2. Sample Processing

Sample processing involved pooled human serum/plasma (e.g., glycoprotein standards purified from human serum/plasma) for assay normalization, dithiothreitol (DTT), and iodoacetamide (IAA), sequencing-grade trypsin, LC-MS-grade water and acetonitrile, and formic acid (LC-MS grade). Serum samples were treated with DTT and IAA to reduce disulfide bonds and to inhibit cysteine proteases, respectively, followed by digestion with trypsin at 37° C. for 18 hours. The digestion was quenched by adding formic acid to each sample to a final concentration of 1% (v/v).

LC-MS analysis included separating digested serum samples over an Agilent ZORBAX Eclipse Plus C18 column (2.1 mm×150 mm i.d., 1.8 μm particle size) using an Agilent 1290 Infinity UHPLC system. The mobile phase A consisted of 3% acetonitrile, 0.1% formic acid in water (v/v), and the mobile phase B of 90% acetonitrile 0.1% formic acid in water (v/v), with the flow rate set at 0.5 mL/minute. The binary solvent composition was set at 100% mobile phase A at the beginning of the run, linearly shifting to 20% B at 20 minutes, 30% B at 40 minutes, and 44% B at 47 minutes. The column was flushed with 100% B and equilibrated with 100% A for a total run time of 70 minutes. After electrospray ionization, operated in positive ion mode, samples were injected into an Agilent 6495B triple quadrupole MS operated in dynamic multiple reaction monitoring (dMRM) mode. The MRM transitions comprised 513 glycopeptide structures which were normalized by comparing them with the abundance of 71 non-glycosylated peptide structures, representing each of 71 proteins from which the glycopeptides monitored were derived. Samples were injected randomized as to underlying phenotype, and reference pooled serum digests were injected interspersed with study samples.

### I.A.3. Data Analysis

Analysis resulted in 683 peptide structures (both peptide and glycopeptide isoforms) being reflected by 1106 MRM transitions, representing 71 high-abundance (concentrations [symbol] of 10 μg/ml) serum glycoproteins. Our transition list consisted of glycopeptides and non-glycosylated peptides from each glycoprotein. A spectrogram feature recognition and integration software based on recurrent neural networks was used to integrate chromatogram peaks and to obtain molecular abundance quantification for each peptide structure.

Normalized abundances of peptide structures, corrected for within-run drift, were assessed in samples from healthy controls, patients with benign pelvic tumors and those with EOC. Raw abundances were normalized by using spiked-in heavy-isotope-labeled internal standards with known peptide concentrations. The calculation relies either on relative abundance or on site occupancy, i.e., on the fractional abundance across all glycans observed at that site. Log-transformed concentration-normalized data for 501 glycopeptide structures (452 of which are based on on-site occupancy and 49 on relative abundance) and for 70 aglycosylated peptide structures were ultimately used for the analysis, totaling 571 unique peptide structures. Fold changes for individual peptide structures were calculated on normalized abundances of healthy (control) vs. EOC samples and benign tumor vs. EOC samples. False discovery rates (FDR) were calculated using the Benjamini-Hochberg method. Principal component analysis (PCA) was performed on log-concentration-normalized abundances of glycopeptide structures to investigate differences among the three phenotypes (e.g., healthy control, EOC, and benign pelvic tumor) studied. Prior to performing PCA, normalized abundances were scaled such that the distributions of all biomarkers were Gaussian with zero mean and unit variance.

To compare any two phenotypes, age-adjusted linear regression was used on a feature-by-feature basis with phenotype serving as the sole binary independent variable. Correcting for multiple comparisons, differences of any biomarker among phenotype groups compared were considered statistically significant where the FDR was less than 0.05. Examples of features include relative abundance (or normalized relative abundance), concentration (or normalized concentration), and site occupancy (fractional abundance across all glycans observed at the corresponding linking site of the corresponding peptide sequence).

For supervised multivariate modeling, a total of 1084 features (571 concentration, 49 relative abundance, and 464 site occupancy features) were log-transformed and split into a training set formed by 80% of all samples from women with benign pelvic tumors and EOC, and a testing set formed by the remaining 20% of these women and all healthy controls. To perform binary classification and predict the probability of EOC, repeated five-fold cross-validated LASSO-regularized logistic regression was used with hyperparameters tuned to prevent overfitting and promote balanced sensitivity and specificity metrics. Training of the binary classification model was performed using the subset of the 1084 total features having low coefficients of variation (<20%) in pooled serum replicates. This subset included 976 features, with each feature being a concentration, relative abundance, or site occupancy for a corresponding peptide structure and where some peptide structures correspond with multiple features. For example, a given peptide structure may be associated with one, two, or three features within the subset of the 976 features.

### I.A.4. Results

Normalized abundances of 428 peptide structures were found to display statistically significantly different abundances (FDR<0.05) in samples of patients with benign pelvic tumors and samples of patients with EOC. 139 peptide structures had statistically significant abundance differences between benign vs. early stage (e.g., stage 1 or 2) EOC. 412 peptide structures had statistically significant abundance differences between benign vs. late stage (e.g., stage 3 or 4) EOC, 137 of which overlapped with those for benign v. early stage. When comparing samples of healthy controls with samples from all EOCs, benign tumors, early stage (e.g., stage 1 or 2) EOC, and late stage (e.g., stage 3 or 4) EOC, statistically significant abundances were found for 386, 149, 215, and 365 markers, respectively. 120 peptide structures were found to be statistically significantly differentially abundant in healthy controls vs. patients with benign pelvic tumors, and in healthy control vs. EOC. 200 peptide structures were found to be statistically significantly differentially abundant in in healthy control vs. early stage EOC and healthy control vs. late stage EOC. Lastly, of the 428 and 386 markers that were found statistically significantly differentially expressed between EOC vs. benign pelvic tumors and EOC vs. healthy controls, respectively, 328 were shared.

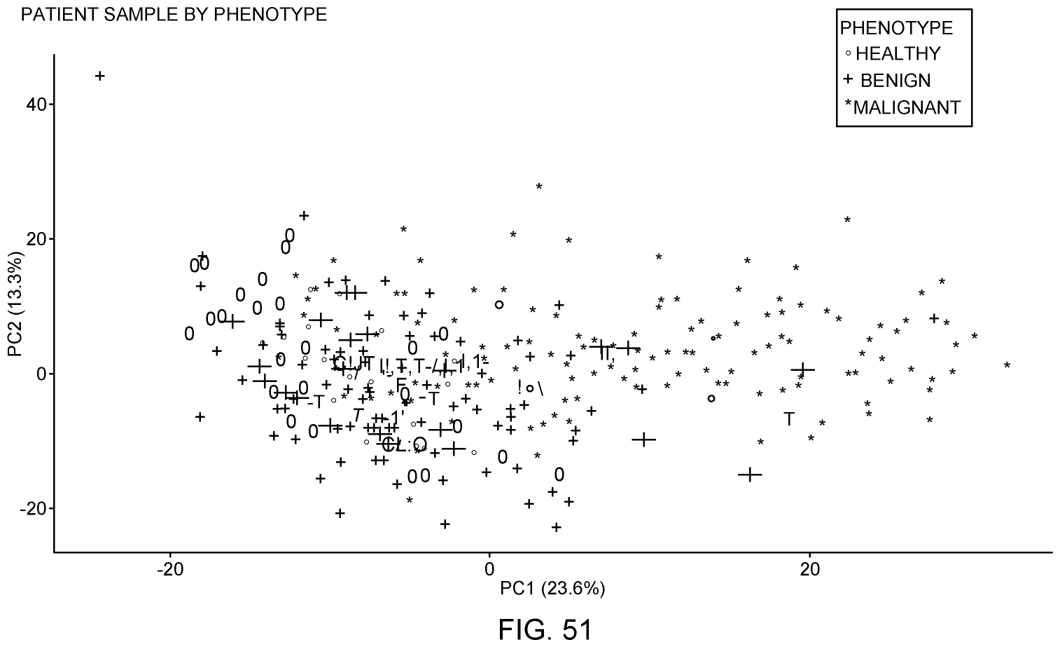

FIG. 51 is a plot diagram illustrating the results of a principal component analysis performed to assess the segregation between healthy, benign pelvic tumor, and EOC samples across first and second principal components in accordance with one or more embodiments. Generally, EOC samples segregated distinctly from healthy control samples, while most benign pelvic tumors did not segregate as distinctly from healthy control samples.

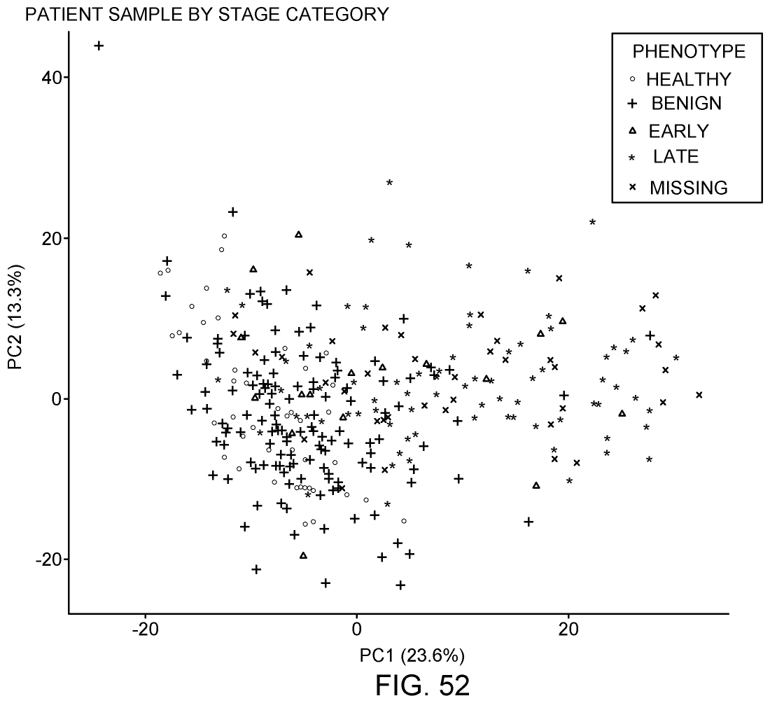

FIG. 52 is a plot diagram illustrating the results of a principal component analysis performed to assess segregation between healthy, benign pelvic tumor, early EOC, late EOC, and missing (undocumented) samples). Generally, EOC samples (and in particular late stage EOC samples) segregated distinctly from healthy control samples, while most benign pelvic tumors did not segregate as distinctly from healthy control samples.

### I.A.5. Results in Context of Screening for Malignant EOC

To assess the suitability of serum glycoproteomics in the context of screening for malignant EOC, a multivariable model was built to predict EOC vs. healthy status. This multivariable model is a supervised machine learning model that includes a logistic regression model, the logistic regression model including a LASSO regression model. Repeated cross-validation in the training set established the optimal LASSO hyperparameter (lambda=0.0608, cross-validated F1=0.971). Applying this amount of shrinkage to the panel of 976 features resulted in a logistic model with 10 peptide structures with non-zero coefficients.

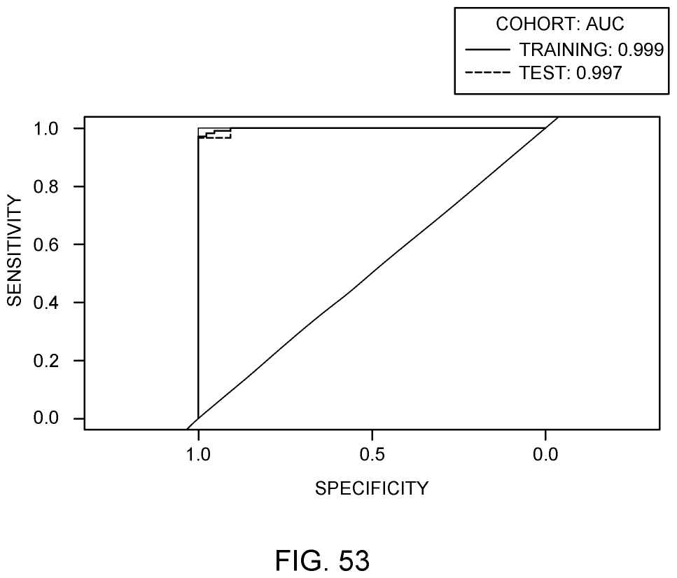

FIG. 53 is an illustration of a receiver operating characteristic (ROC) diagram corresponding to the multivariable model built to predict malignancy v. benign status of pelvic tumors in accordance with one or more embodiments. The multivariable model achieved high accuracy in both the training set (accuracy=0.975, sensitivity=0.983, specificity=0.955) and the test set (accuracy=0.976, sensitivity=0.967, specificity=1.0). Further, ROC analysis demonstrated strong performance across a range of cutoffs, and little overfitting, with the training AUC (area under the curve)=0.999 and test AUC=0.997.

Thus, the multivariable model that was built may be used accurately and reliably to malignant EOC and distinguish such malignancy from a healthy status. Such diagnostic power may be used to reduce the need for unnecessary invasive testing. Further, such diagnostic information can be used to identify patients with EOC earlier, which may lead to earlier treatment, improved treatment recommendations, and improved treatment plans.

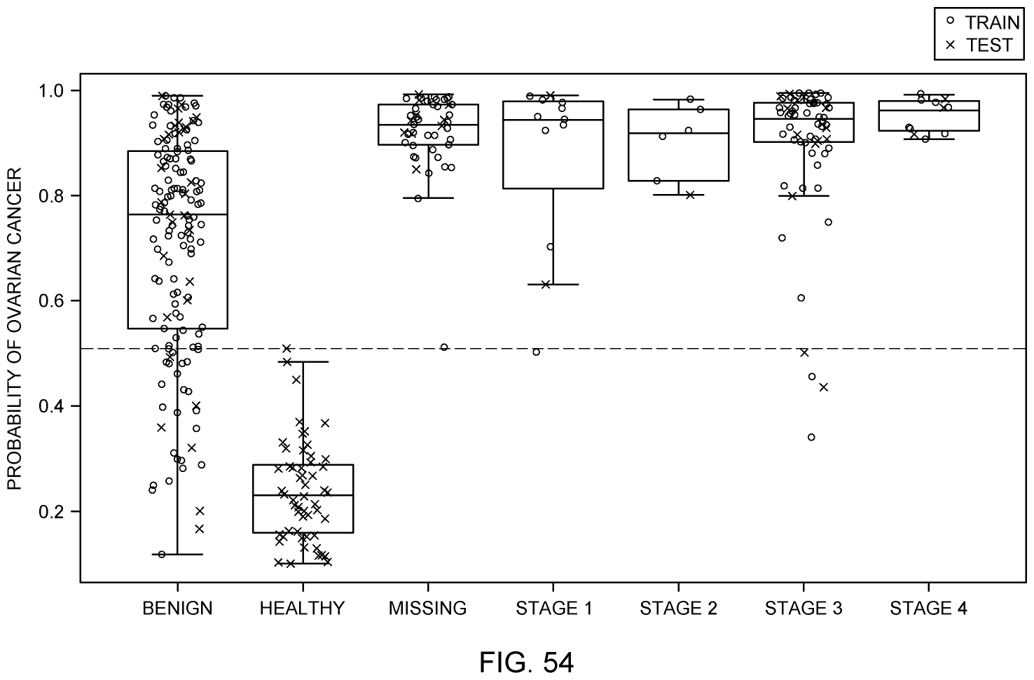

FIG. 54 is an illustration of a diagram showing the probability distributions for the various groups using the multivariable model for predicting malignancy v. benign status of pelvic tumors in accordance with one or more embodiments. As shown in FIG. 54, the probability distributions for benign pelvic tumor, healthy, missing (undocumented), stage 1 EOC, stage 2 EOC, stage 3 EOC, and stage 4 EOC samples increased with cancer stage, with probability distributions being similar across training and test sets. Notably, applying the built multivariable model to healthy patients, who were not utilized in the training, resulted in few misclassifications and a spread nearly equivalent to that of the benign pelvic tumor cases. Such results indicate that the glycoproteomic signature of the solidly predicts malignancy and severity of disease.

Table 48 below provides the fold changes, FDRs, and p-values for the 10 peptide structures PS-165 to PS-174 (same as those in Table 41 above) based on differential expression analysis (DEA). The peptide structures PS-165 to PS-174 are ordered both in Table 41 and in Table 48 with respect to relative significance to the probability score generated by the model. More significant peptide structures had higher coefficients in the LASSO regression model, while less significant peptide structures had lower coefficients in the LASSO regression model. In other words, relative significance to the probability score decreased with decreasing coefficients. Further, each peptide structure is associated with a feature that was used for the model (relab=relative abundance; conc=concentration).

TABLE 48
Peptide Structure Markers for Regression Model to distinguish
between Epithelial Ovarian Cancer and Healthy State

| | | Healthy v | Healthy v | Healthy v | |
|---|---|---|---|---|---|
| PS- | | EOC | EOC | EOC | |
| ID NO. | PS-NAME | (Fold Change) | (FDR) | (p-value) | Feature |
| 165 | ZA2G_128_5402 | 1.57212 | 1.99E−13 | 3.14E−15 | relab |
| 166 | IC1_253_6503 | 2.26917 | 6.42E−18 | 2.25E−20 | conc |
| 167 | CFAI_494_5402 | 1.30391 | 3.00E−07 | 4.78E−08 | relab |
| 168 | CERU_138_6513 | 1.37235 | 2.14E−06 | 4.85E−07 | relab |
| 169 | IGG1_297_3410 | 1.98807 | 1.03E−09 | 6.47E−11 | conc |
| 170 | HEMO_64_5402 | 1.53316 | 3.06E−11 | 1.12E−12 | relab |
| 171 | APOB_983_5402 | 1.98566 | 1.11E−13 | 1.17E−15 | conc |
| CK-1 | FINC_SYTITGLQPGTDYK | 0.51932 | 9.92e−09 | 1.043e−09 | relab |
| 172 | HPT_207_121005 | 2.21826 | 3.17E−10 | 1.66E−11 | conc |
| 173 | IGG3_297_3400 | N/A | N/A | N/A | relab |
| 174 | IGG34_297_3400 | N/A | N/A | N/A | relab |
| CK-2 | APOM_135_8500_CHK | 0.59098 | 1.58e−17 | 8.28e−20 | conc |

### I.A.6. Results in Context of Triaging Pelvic Tumors

To assess the suitability of serum glycoproteomics in the context of clinically triaging pelvic tumors, a multivariable model was built to predict malignancy vs. benign status of such pelvic tumors. This multivariable model is a supervised machine learning model that includes a logistic regression model, the logistic regression model including a LASSO regression model. Repeated cross-validation in the training set established the optimal LASSO hyperparameter (lambda=0.045, cross-validated F1=0.849). Applying this amount of shrinkage to the panel of 976 features resulted in a logistic model with 25 peptide structures with non-zero coefficients.

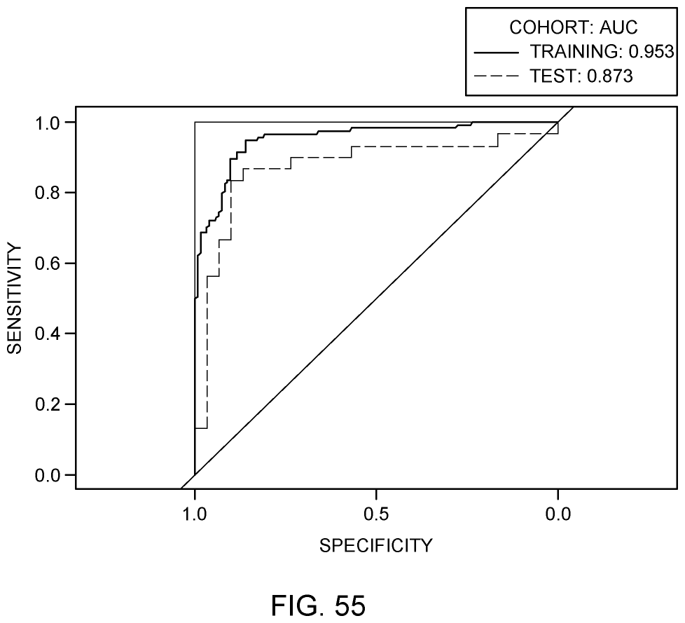

FIG. 55 is an illustration of a receiver operating characteristic (ROC) diagram corresponding to the multivariable model built to predict malignancy v. benign status of pelvic tumors in accordance with one or more embodiments. The multivariable model achieved high accuracy in both the training set (accuracy=0.869, sensitivity=0.835, specificity=0.901) and the test set (accuracy=0.867, sensitivity=0.867, specificity=0.867). Further, ROC analysis demonstrated strong performance across a range of cutoffs, and little overfitting, with the training AUC (area under the curve)=0.953 and test AUC=0.873.

Thus, the multivariable model that was built may be used accurately and reliably to triage pelvic tumors and distinguish those that are malignant from those that are benign. Such diagnostic power may be used to reduce the need for invasive testing (e.g., biopsy) prior to treatment can be administered. Further, such diagnostic information can be used to improve treatment recommendations and treatment plans (e.g., earlier treatment in the case of malignant EOC) and reduce indications for unnecessary treatment (e.g., no indication for surgery when the pelvic tumor is benign).

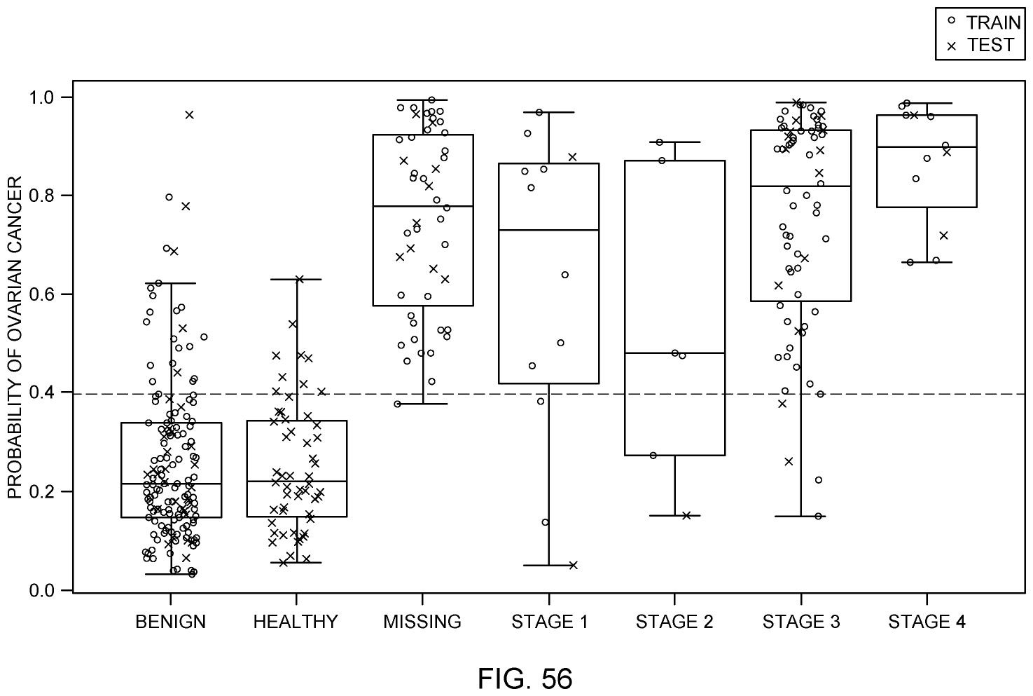

FIG. 56 is an illustration of a diagram showing the probability distributions for the various groups using the multivariable model for predicting malignancy v. benign status of pelvic tumors in accordance with one or more embodiments. As shown in FIG. 54, the probability distributions for benign pelvic tumor, healthy, missing (undocumented), stage 1 EOC, stage 2 EOC, stage 3 EOC, and stage 4 EOC samples increased with cancer stage, with probability distributions being similar across training and test sets. Notably, applying the built multivariable model to healthy patients, who were not utilized in the training, resulted in few misclassifications and a spread nearly equivalent to that of the benign pelvic tumor cases. Such results indicate that the glycoproteomic signature of the 25 peptide structures for the LASSO regression model solidly predict malignancy and severity of disease.

Table 49 below provides the fold changes, FDRs, and p-values for the 25 peptide structures PS-169 and PS-175 to PS-198 (same as those in Table 42 above) based on differential expression analysis (DEA). The peptide structures PS-169 and PS-175 to PS-198 are ordered both in Table 42 and in Table 49 with respect to relative significance to the probability score generated by the model. More significant peptide structures had higher coefficients in the LASSO regression model, while less significant peptide structures had lower coefficients in the LASSO regression model. In other words, relative significance to the probability score decreased with decreasing coefficients. Further, each peptide structure is associated with a feature that was used for the model (relab=relative abundance; conc=concentration).

TABLE 49
Peptide Structure Markers for Regression Model to distinguish
between Epithelial Ovarian Cancer and Benign Pelvic Tumor

| | | Benign v. | | | |
|---|---|---|---|---|---|
| | | EOC | Benign v. | | |
| PS-ID | | (Fold | EOC | Benign v. EOC | |
| NO. | PS-NAME | Change) | (FDR) | (p-value) | Feature |
| CK-3 | APOD_98_9800_CHECK | 1.54848 | 4.78e−13 | 8.46e−14 | relab |
| 175 | CO2_621_5200 | 1.36880 | 1.73E−11 | 3.66E−12 | relab |
| 169 | IGG1_297_3410 | 1.54336 | 2.47E−10 | 6.61E−11 | relab |
| 176 | AGP1_93_7612 | 2.39546 | 2.79E−16 | 2.20E−17 | relab |
| 177 | AACT_271_7602 | 1.68006 | 2.27E−08 | 7.70E−09 | conc |
| 178 | A2MG_1424_5402 | 1.15594 | 0.007733584 | 0.005106062 | relab |
| 179 | AACT_271_6513 | 2.34075 | 2.81E−18 | 1.04E−19 | relab |
| 180 | CERU_397_5402 | 1.07300 | 0.008195667 | 0.005425503 | relab |
| 181 | APOB_3411_5301 | 1.01808 | 0.743228938 | 0.714593147 | relab |
| 182 | AACT_106_6513 | 2.11211 | 1.42E−16 | 9.67E−18 | relab |
| 183 | CERU_138_5402 | 1.08927 | 0.002831028 | 0.001760096 | conc |
| 184 | A1AT_107_6513 | 2.15635 | 6.82E−14 | 1.06E−14 | relab |
| 185 | AGP1_93_7602 | 1.11780 | 0.012740002 | 0.008679266 | relab |
| 186 | VTNC_242_6502 | 0.83257 | 0.000446981 | 0.000252845 | relab |
| 187 | IGG2_297_3510 | 0.69463 | 8.28E−10 | 2.36E−10 | conc |
| 188 | CFAH_882_5411 | 0.84102 | 1.06E−05 | 4.78E−06 | relab |
| CK-4 | APOM_135_8500_CHECK | 0.81884 | 1.16e−08 | 3.87e−09 | conc |
| 189 | AGP1_103_8704 | 1.18615 | 0.001152856 | 0.000676369 | relab |
| 190 | IGG1_297_4300 | 0.60088 | 2.09E−11 | 4.47E−12 | relab |
| 191 | APOH_253_5401 | 0.62217 | 1.65E−16 | 1.16E−17 | conc |
| 192 | APOD_98_5411 | 0.71180 | 1.50E−12 | 2.82E−13 | conc |
| 193 | TRFE_630_5411 | 0.69298 | 4.01E−14 | 5.62E−15 | conc |
| 194 | CERU_138_6502 | 0.81476 | 7.13E−07 | 2.87E−07 | relab |
| 195 | A2MG_1424_5411 | 0.67638 | 1.53E−23 | 2.68E−26 | conc |
| 196 | A2MG_55_5411 | 0.71212 | 2.20E−20 | 1.93E−22 | conc |
| 197 | TRFE_630_5412 | 0.77453 | 1.01E−09 | 2.95E−10 | conc |
| 198 | IGG2_297_4511 | 0.73039 | 3.50E−08 | 1.23E−08 | conc |

Table 50A below provides the fold changes, FDRs, and p-values forthe 36 peptide structures PS-168, PS-172, PS-182, PS-200, PS-201, PS-205, PS-220, PS-226 to PS-254 (same as those in Table 43B above) based on differential expression analysis (DEA). The peptide structures PS-168, PS-172, PS-182, PS-200, PS-201, PS-205, PS-220, PS-226 to PS-254 are ordered in Table 50A with respect to relative significance to the p value score generated by the model.

TABLE 50A
Peptide Structure Markers for Regression Model to
distinguish between late stage (3/4) EOC and early
stage EOC (1/2) using the biomarkers of Table 43B.

| | | Benign v. | | |
|---|---|---|---|---|
| | | EOC | Benign v. | |
| PS-ID | | (Fold | EOC | Benign v. EOC |
| NO. | PS-NAME | Change) | (FDR) | (p-value) |
| 230 | AGP1_103_6513 | 1.539 | 0.0001 | 1.22E−06 |
| 253 | KNG1_294_6503 | 0.817 | 0.0006 | 1.94E−05 |
| 205 | TRFE_630_6513 | 1.361 | 0.001 | 3.71E−05 |
| 249 | HPT_207_11915 | 1.469 | 0.0011 | 4.62E−05 |
| 232 | AGP1_103_7614 | 1.425 | 0.0011 | 5.35E−05 |
| 172 | HPT_207_121005 | 0.736 | 0.0013 | 7.32E−05 |
| 247 | HPT_184_6513 | 1.575 | 0.002 | 0.0001 |
| 250 | HPT_241_6513 | 1.64 | 0.0022 | 0.0002 |
| 253 | KNG1_205_6513 | 1.512 | 0.0028 | 0.0002 |
| 235 | AGP1_93_7613 | 1.436 | 0.0031 | 0.0003 |
| 236 | AGP12_56_6503 | 0.78 | 0.0031 | 0.0003 |
| 238 | AGP12_72_6503 | 0.759 | 0.0034 | 0.0003 |
| 245 | HEMO_187_6503 | 0.861 | 0.0034 | 0.0003 |
| 241 | APOH_162_6503 | 0.799 | 0.0035 | 0.0003 |
| 251 | HPT_241_7613 | 1.58 | 0.0039 | 0.0004 |
| 235 | AGP1_93_6513 | 1.398 | 0.0039 | 0.0004 |
| 243 | CERU_762_6513 | 1.19 | 0.0039 | 0.0004 |
| 201 | AGP1_93_7614 | 1.327 | 0.0039 | 0.0004 |
| 226 | AACT_106_6503 | 0.749 | 0.0045 | 0.0005 |
| 229 | AGP1_103_6503 | 0.792 | 0.0045 | 0.0006 |
| 244 | FETUA_156_6503 | 0.837 | 0.0045 | 0.0006 |
| 228 | AACT_271_6502 | 0.688 | 0.0046 | 0.0006 |
| 254 | VTNC_242_6503 | 0.836 | 0.0049 | 0.0007 |
| 231 | AGP1_103_7602 | 0.767 | 0.0049 | 0.0007 |
| 220 | AACT_106_7614 | 1.379 | 0.0051 | 0.0007 |
| 240 | AGP2_103_6513 | 1.438 | 0.0053 | 0.0008 |
| 227 | AACT_106_7604 | 0.794 | 0.0056 | 0.0009 |
| 237 | AGP12_56_6513 | 1.426 | 0.0065 | 0.001 |
| 182 | AACT_106_6513 | 1.336 | 0.0096 | 0.0017 |
| 246 | HEMO_187_6513 | 1.232 | 0.0107 | 0.002 |
| 233 | AGP1_93_6503 | 0.796 | 0.0112 | 0.0022 |
| 200 | FETUA_176_6513 | 1.394 | 0.0123 | 0.0025 |
| 168 | CERU_138_6513 | 1.143 | 0.0253 | 0.0066 |
| 239 | AGP12_72_7603 | 0.835 | 0.0259 | 0.0069 |
| 242 | CERU_397_6513 | 1.225 | 0.0375 | 0.0113 |
| 248 | HPT_207_11904 | 0.865 | 0.0386 | 0.0117 |

Table 50B below provides the fold changes, FDRs, and p-values for the 25 peptide structures denoted by SEQ ID NO 475-499 (in accordance with Table 43C above) using differential expression analysis (DEA).

TABLE 50B
Peptide Structure Markers for Regression Model to distinguish between late stage
(3/4) EOC and early stage EOC (1/2) using the biomarkers of Table 43C.

| | | stage 3, 4 vs | stage 3, 4 vs | stage 3, 4 vs |
|---|---|---|---|---|
| SEQ ID | | stage 1, 2 | stage 1, 2 | stage 1, 2 |
| NO | PS-NAME | (fold_change) | (FDR) | (p-value) |
| 475 | AACT_271_6512 | 0.704 | 0.0028 | 0.0002 |
| 476 | AGP1_103_6503 | 0.792 | 0.0045 | 0.0006 |
| 477 | AGP1_103_6513 | 1.539 | 0.0001 | 1.22E−06 |
| 478 | AGP1_103_7614 | 1.425 | 0.0011 | 5.35E−05 |
| 479 | AGP1_93_6513 | 1.398 | 0.0039 | 0.0004 |
| 480 | AGP1_93_7604 | 1.471 | 0.001 | 3.94E−05 |
| 481 | AGP1_93_7613 | 1.436 | 0.0031 | 0.0003 |
| 482 | AGP1_93_7614 | 1.327 | 0.0039 | 0.0004 |
| 483 | AGP12_56_6503 | 0.78 | 0.0031 | 0.0003 |
| 484 | AGP12_72_6503 | 0.759 | 0.0034 | 0.0003 |
| 485 | AGP12_72_7604 | 1.546 | 0.0012 | 6.85E−05 |
| 486 | AGP12_72MC_7604 | 1.592 | 0.0013 | 8.30E−05 |
| 487 | AGP12_72MC_7614 | 1.464 | 0.002 | 0.0001 |
| 488 | APOH_162_6503 | 0.799 | 0.0035 | 0.0003 |
| 489 | CERU_762_6513 | 1.19 | 0.0039 | 0.0004 |
| 490 | HEMO_187_6503 | 0.861 | 0.0034 | 0.0003 |
| 491 | HPT_184_6513 | 1.575 | 0.002 | 0.0001 |
| 492 | HPT_207_11915 | 1.469 | 0.0011 | 4.62E−05 |
| 493 | HPT_207_121005 | 0.736 | 0.0013 | 7.32E−05 |
| 494 | HPT_241_6503 | 1.607 | 0.001 | 4.78E−05 |
| 495 | HPT_241_6513 | 1.64 | 0.0022 | 0.0002 |
| 496 | HPT_241_7613 | 1.58 | 0.0039 | 0.0004 |
| 497 | KNG1_205_6513 | 1.512 | 0.0028 | 0.0002 |
| 498 | KNG1_294_6503 | 0.817 | 0.0006 | 1.94E−05 |
| 499 | TRFE_630_6513 | 1.361 | 0.001 | 3.71E−05 |

Table 50C below provides the fold changes, FDRs, and p-values for the 50 peptide structures denoted by SEQ ID NO 500-549 (in accordance with Table 43D above) using differential expression analysis (DEA). For this differential expression analysis, the subjects from EOC stages 1-4 were used for this analysis as shown in the table of FIG. 50. EOC stages 1 and 2 (12+6) were combined to be the early stage cohort and EOC stages 3 and 4 (68+12) were combined to be the late stage cohort.

TABLE 50C
Peptide Structure Markers for Regression Model to distinguish between late stage
(3/4) EOC and early stage EOC (1/2) using the biomarkers of Table 43D.

| | | stage 3, 4 vs | stage 3, 4 vs | stage 3, 4 vs |
|---|---|---|---|---|
| SEQ ID | | stage 1, 2 | stage 1, 2 | stage 1, 2 |
| NO | PS-Name | (fold_change) | (FDR) | (p-value) |
| 500 | A1AT_107_6512 | 1.319 | 0.294 | 0.454 |
| 501 | A2MG_1424_6501 | 1.248 | 0.129 | 0.274 |
| 502 | A2MG_1424_6511 | 1.025 | 0.776 | 0.868 |
| 503 | AACT_106_6503 | 0.739 | 0.035 | 0.132 |
| 504 | AACT_106_7604 | 0.789 | 0.040 | 0.148 |
| 505 | AACT_106_7614 | 1.319 | 013 | 0.076 |
| 506 | AACT_271_6502 | 0.587 | 0.004 | 0.056 |
| 507 | AACT_271_6503 | 0.663 | 0.013 | 0.076 |
| 508 | AGP12_56_6503 | 0.746 | 0.005 | 0.059 |
| 509 | AGP12_72MC_6503 | 0.841 | 0.099 | 0.239 |
| 510 | AGP12_72MC_6513 | 1.389 | 0.003 | 0.054 |
| 511 | AGP12_72MC_7613 | 1.561 | 0.001 | 0.035 |
| 512 | AGP12_72MC_7614 | 1.669 | 0.001 | 0.035 |
| 513 | AGP12_72_6503 | 0.685 | 0.005 | 0.061 |
| 514 | AGP12_72_7603 | 0.769 | 0.027 | 0.115 |
| 515 | AGP12_72_7604 | 0.835 | 0.083 | 0.22 |
| 516 | AGP1_103_6503 | 0.838 | 0.125 | 0.269 |
| 517 | AGP1_103_6513 | 1.481 | <0.001 | 0.013 |
| 518 | AGP1_103_7602 | 0.705 | 0.013 | 0.076 |
| 519 | AGP1_103_7604 | 0.866 | 0.245 | 0.408 |
| 520 | AGP1_103_7614 | 1.386 | <0.001 | 0.013 |
| 521 | AGP1_93_6503 | 0.828 | 0.125 | 0.269 |
| 522 | AGP1_93_7602 | 0.854 | 0.182 | 0.3417 |
| 523 | AGP1_93_7603 | 0.887 | 0.243 | 0.404 |
| 524 | AGP1_93_7604 | 0.951 | 0.624 | 0.761 |
| 525 | AGP1_93_7612 | 1.293 | 0.031 | 0.126 |
| 526 | AGP1_93_7613 | 1.417 | 0.001 | 0.03 |
| 527 | AGP1_93_7614 | 1.508 | <0.001 | 0.015 |
| 528 | AGP2_103_6503 | 0.805 | 0.094 | 0.232 |
| 529 | AGP2_103_6513 | 1.386 | <0.001 | 0.015 |
| 530 | AGP2_103_7604 | 0.836 | 0.173 | 0.334 |
| 531 | APOH_162_6503 | 0.856 | 0.099 | 0.239 |
| 532 | APOH_253_5412 | 1.278 | 0.0185 | 0.093 |
| 533 | CERU_397_6513 | 1.323 | 0.012 | 0.076 |
| 534 | CERU_762_6503 | 0.814 | 0.019 | 0.095 |
| 535 | FETUA_156_6503 | 0.856 | 0.043 | 0.155 |
| 536 | FETUA_156_6513 | 1.155 | 0.222 | 0.387 |
| 537 | FETUA_176_6513 | 1.358 | 0.088 | 0.228 |
| 538 | HEMO_187_6503 | 0.882 | 0.056 | 0.175 |
| 539 | HEMO_187_6513 | 1.214 | 0.044 | 0.155 |
| 540 | HPT_184_6513 | 1.50 | 0.032 | 0.130 |
| 541 | HPT_207_11904 | 0.812 | 0.038 | 0.141 |
| 542 | HPT_207_11914 | 1.253 | 0.0128 | 0.076 |
| 543 | HPT_207_11915 | 1.55 | <0.001 | 0.015 |
| 544 | HPT_207_121005 | 0.742 | 0.0136 | 0.077 |
| 545 | HPT_241_6511 | 1.217 | 0.171 | 0.33 |
| 546 | HPT_241_6512 | 1.595 | 0.01 | 0.07 |
| 547 | HPT_241_6513 | 1.629 | 0.007 | 0.065 |
| 548 | HPT_241_7613 | 1.506 | 0.053 | 0.172 |
| 549 | KNG1_294_6503 | 0.8 | 0.003 | 0.054 |

The markers from Table 43B were used to train a regularized regression model (e.g., LASSO regression model). Coefficients for the regularized regression model (e.g., LASSO regression model) are provided in Table 51A. Using the values of Table 51A, a probability for one of the states can be determined by summing together the product of the concentration of each biomarker in the sample and the respective coefficient (of one column) and then adding the summation and the intercept to yield the logit of a probability score. For example, the logit of the probability, to which the inverse logit function can be applied, is equal to the following equation 1 (eq. 1).

$${\sum }_{i = 1}^{i = n} [ ( {Concentration}_{PS - ID NO : i} ) \times ( {Coefficient}_{PS - ID NO : i} ) ] + Intercept \\ where n = a number of biomarkers having a unique PS - ID No , \\ i = an index number for each of the biomarkers , \qquad ( eq . 1 )$$

TABLE 51A
Coefficients for Peptide Structure Markers for a Regression
Model to distinguish between late stage (3/4) EOC and early
stage EOC (1/2) based on the biomarkers of Table 43B

| | | Coefficients |
|---|---|---|
| | | (stage 3, 4 vs stage |
| PS-ID No | PS-NAME | 1, 2) |
| 226 | AACT_106_6503 | 0.789963 |
| 182 | AACT_106_6513 | −0.100283 |
| 227 | AACT_106_7604 | −0.23972 |
| 220 | AACT_106_7614 | −0.174242 |
| 228 | AACT_271_6502 | −1.019336 |
| 229 | AGP1_103_6503 | −1.129647 |
| 230 | AGP1_103_6513 | 2.450196 |
| 231 | AGP1_103_7602 | −0.826335 |
| 232 | AGP1_103_7614 | 0.342801 |
| 233 | AGP1_93_6503 | 0.888268 |
| 234 | AGP1_93_6513 | −1.638508 |
| 235 | AGP1_93_7613 | −0.120831 |
| 201 | AGP1_93_7614 | −0.94578 |
| 236 | AGP12_56_6503 | −0.211526 |
| 237 | AGP12_56_6513 | 0.105946 |
| 238 | AGP12_72_6503 | −1.586863 |
| 239 | AGP12_72_7603 | 0.544458 |
| 240 | AGP2_103_6513 | −0.132156 |
| 241 | APOH_162_6503 | 0.465722 |
| 168 | CERU_138_6513 | |
| 242 | CERU_397_6513 | −1.056969 |
| 243 | CERU_762_6513 | 1.538329 |
| 244 | FETUA_156_6503 | −0.478039 |
| 200 | FETUA_176_6513 | 0.343385 |
| 245 | HEMO_187_6503 | −0.012419 |
| 246 | HEMO_187_6513 | −2.077228 |
| 247 | HPT_184_6513 | 1.018212 |
| 248 | HPT_207_11904 | 0.950984 |
| 249 | HPT_207_11915 | 1.289189 |
| 172 | HPT_207_121005 | −0.387854 |
| 250 | HPT_241_6513 | −0.890611 |
| 251 | HPT_241_7613 | 0.311607 |
| 252 | KNG1_205_6513 | 0.287132 |
| 253 | KNG1_294_6503 | 0.188466 |
| 205 | TRFE_630_6513 | −0.19935 |
| 254 | VTNC_242_6503 | −0.11461 |

The markers from Table 43C were used to train a regularized regression model (e.g., LASSO regression model). Coefficients for the regularized regression model (e.g., LASSO regression model) are provided in Table 51B. Using the values of Table 51B, a probability for one of the states can be determined by summing together the product of the concentration of each biomarker in the sample and the respective coefficient (of one column) and then adding the summation and the intercept to yield the logit of a probability score (see equation 1).

TABLE 51B
Coefficients for Peptide Structure Markers for a Regression
Model to distinguish between late stage (3/4) EOC and early
stage EOC (1/2) based on the biomarkers of Table 43C.

| SEQ | | |
|---|---|---|
| ID NO | PS-NAME | Coefficients |
| 475 | AACT_271_6512 | 0.267422 |
| 476 | AGP1_103_6503 | −0.44212 |
| 477 | AGP1_103_6513 | 1.735177 |
| 478 | AGP1_103_7614 | 0.240767 |
| 479 | AGP1_93_6513 | −1.34581 |
| 480 | AGP1_93_7604 | 0.346537 |
| 481 | AGP1_93_7613 | −0.33861 |
| 482 | AGP1_93_7614 | −1.4923 |
| 483 | AGP12_56_6503 | −0.75867 |
| 484 | AGP12_72_6503 | −0.57017 |
| 485 | AGP12_72_7604 | 0.214311 |
| 486 | AGP12_72MC_7604 | −0.42256 |
| 487 | AGP12_72MC_7614 | 0.675864 |
| 488 | APOH_162_6503 | 0.124674 |
| 489 | CERU_762_6513 | 0.180667 |
| 490 | HEMO_187_6503 | −0.35174 |
| 491 | HPT_184_6513 | 0.895905 |
| 492 | HPT_207_11915 | 0.362611 |
| 493 | HPT_207_121005 | −0.17095 |
| 494 | HPT_241_6503 | 0.26564 |
| 495 | HPT_241_6513 | −2.44213 |
| 496 | HPT_241_7613 | −0.0651 |
| 497 | KNG1_205_6513 | 0.318803 |
| 498 | KNG1_294_6503 | −0.02677 |
| 499 | TRFE_630_6513 | 0.782865 |

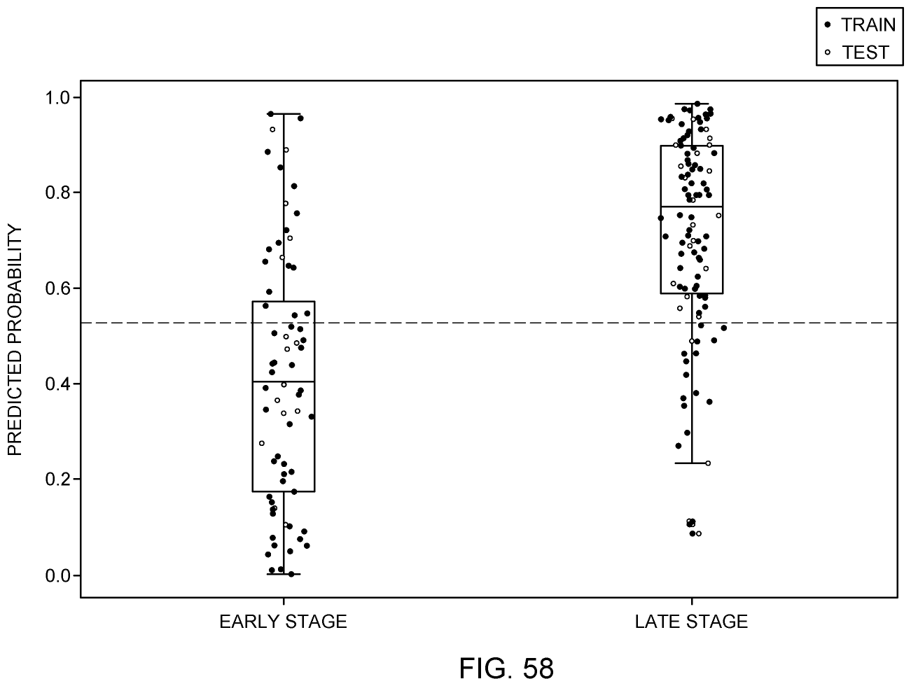

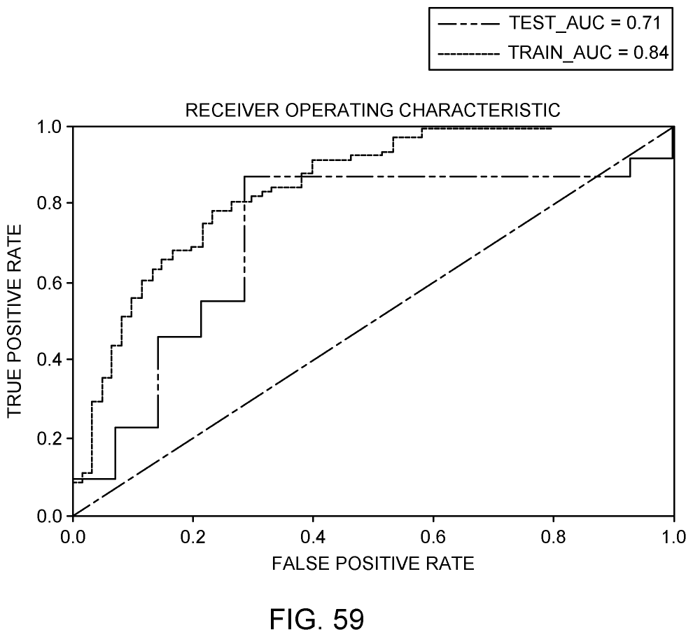

Using the model coefficients of Table 51B, a predicted probability was generated for early stage and late stage ovarian cancer samples showing a stratification in predicted probabilities between the two cohorts as is illustrated in FIG. 58. FIG. 59 illustrates a receiver-operating-characteristic (ROC) curve and the area under curve (AUC) for the regularized regression model (e.g., LASSO regression model) for early stage and late stage ovarian cancer samples using testing case data and training case data. Table 52 shows the accuracy, sensitivity, specificity and precision for the training data set and the testing data set.

Table 53 shows the training accuracy and testing accuracy for the early stage and late stage cohort for ovarian cancer.

TABLE 52

| | Train | Test |
|---|---|---|
| Accuracy | 0.753 | 0.778 |
| Sensitivity | 0.802 | 0.864 |
| Specificity | 0.7 | 0.714 |
| Precision | 0.712 | 0.769 |

TABLE 53

| | Train | Test |
|---|---|---|
| | (accuracy) | (accuracy) |
| Early Stage | 0.7 | 0.714286 |
| Late Stage | 0.802326 | 0.863636 |

The markers from Table 43D were used to train a regularized regression model (e.g., LASSO regression model). Coefficients for the regularized regression model (e.g., LASSO regression model) are provided in Table 51C. Using the values of Table 51C, a probability for one of the states can be determined by summing together the product of the concentration of each biomarker in the sample and the respective coefficient (of one column) and then adding the summation and the intercept to yield the logit of a probability score (see equation 1).

TABLE 51C
Coefficients for Peptide Structure Markers for a Regression
Model to distinguish between late stage (3/4) EOC and early
stage EOC (1/2) based on the biomarkers of Table 43D.

| SEQ | | |
|---|---|---|
| ID NO | PS-Name | Coefficients |
| 500 | A1AT_107_6512 | 0 |
| 501 | A2MG_1424_6501 | 0 |
| 502 | A2MG_1424_6511 | 0 |
| 503 | AACT_106_6503 | 0 |
| 504 | AACT_106_7604 | −0.0022402 |
| 505 | AACT_106_7614 | −0.652235354 |
| 506 | AACT_271_6502 | −0.680231822 |
| 507 | AACT_271_6503 | −1.169551 |
| 508 | AGP12_56_6503 | −2.2045339 |
| 509 | AGP12_72MC_6503 | 0.56949569 |
| 510 | AGP12_72MC_6513 | 0 |
| 511 | AGP12_72MC_7613 | −2.2203046 |
| 512 | AGP12_72MC_7614 | 0 |
| 513 | AGP12_72_6503 | −1.5094621 |
| 514 | AGP12_72_7603 | 0.91786725 |
| 515 | AGP12_72_7604 | 0 |
| 516 | AGP1_103_6503 | 0 |
| 517 | AGP1_103_6513 | 1.51746308 |
| 518 | AGP1_103_7602 | 0 |
| 519 | AGP1_103_7604 | 0 |
| 520 | AGP1_103_7614 | 0 |
| 521 | AGP1_93_6503 | 0 |
| 522 | AGP1_93_7602 | 0.05936161 |
| 523 | AGP1_93_7603 | 1.69720204 |
| 524 | AGP1_93_7604 | 0 |
| 525 | AGP1_93_7612 | 0 |
| 526 | AGP1_93_7613 | 0 |
| 527 | AGP1_93_7614 | 0 |
| 528 | AGP2_103_6503 | 0 |
| 529 | AGP2_103_6513 | 0.15270371 |
| 530 | AGP2_103_7604 | 0 |
| 531 | APOH_162_6503 | 0 |
| 532 | APOH_253_5412 | 0.84222935 |
| 533 | CERU_397_6513 | 0.07304956 |
| 534 | CERU_762_6503 | −0.106671 |
| 535 | FETUA_156_6503 | 0.50770712 |
| 536 | FETUA_156_6513 | −1.2528564 |
| 537 | FETUA_176_6513 | 0 |
| 538 | HEMO_187_6503 | 0 |
| 539 | HEMO_187_6513 | 0 |
| 540 | HPT_184_6513 | 0.25532311 |
| 541 | HPT_207_11904 | 0 |
| 542 | HPT_207_11914 | 0 |
| 543 | HPT_207_11915 | 0 |
| 544 | HPT_207_121005 | 0 |
| 545 | HPT_241_6511 | 0.50726531 |
| 546 | HPT_241_6512 | 0 |
| 547 | HPT_241_6513 | 0 |
| 548 | HPT_241_7613 | 0 |
| 549 | KNG1_294_6503 | 0 |

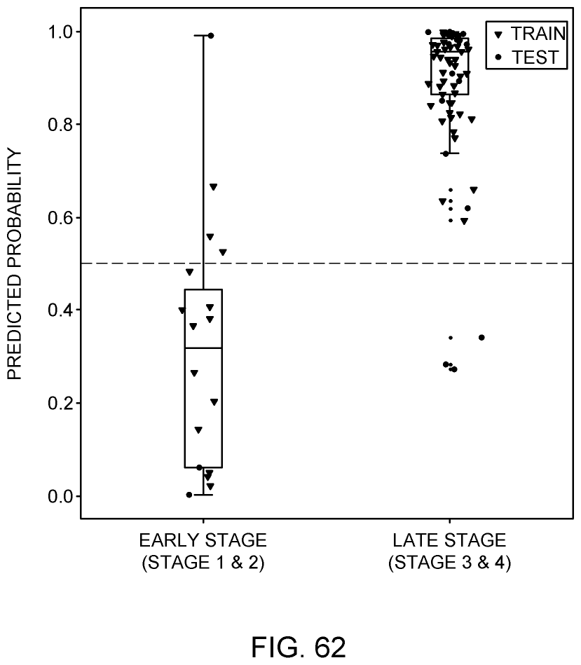

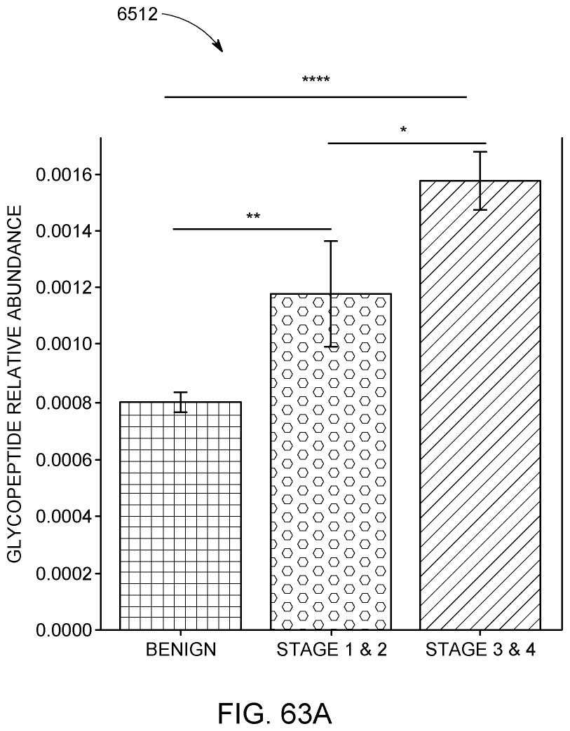

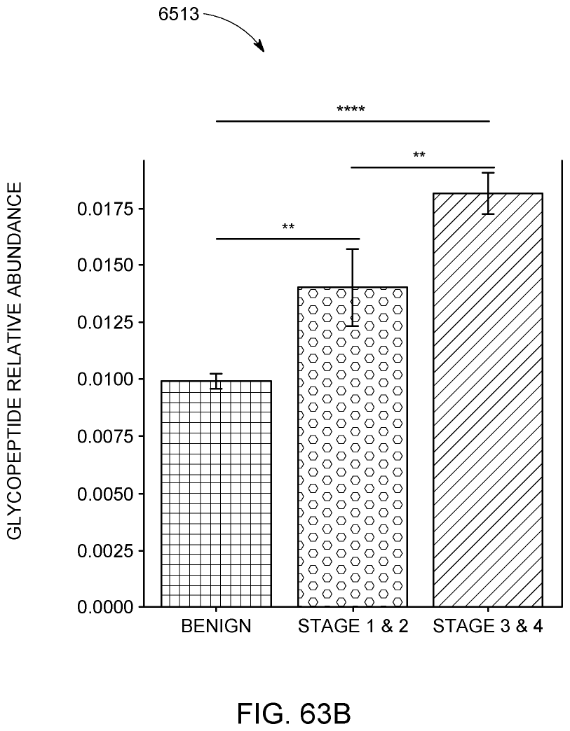

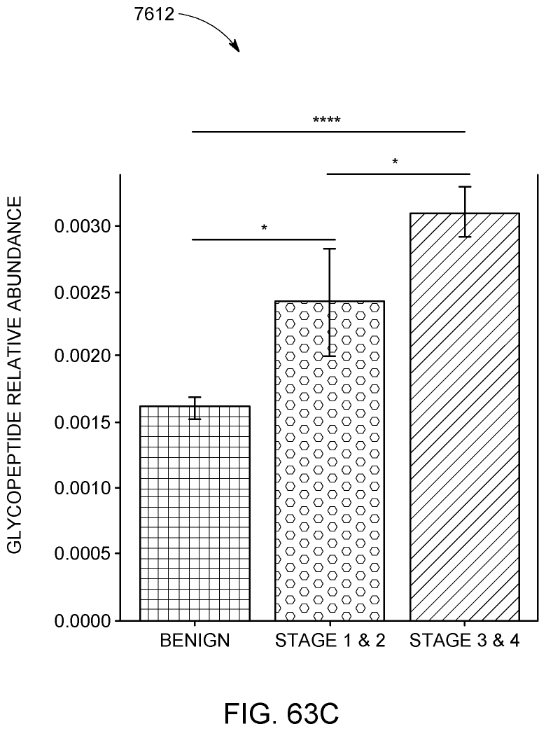

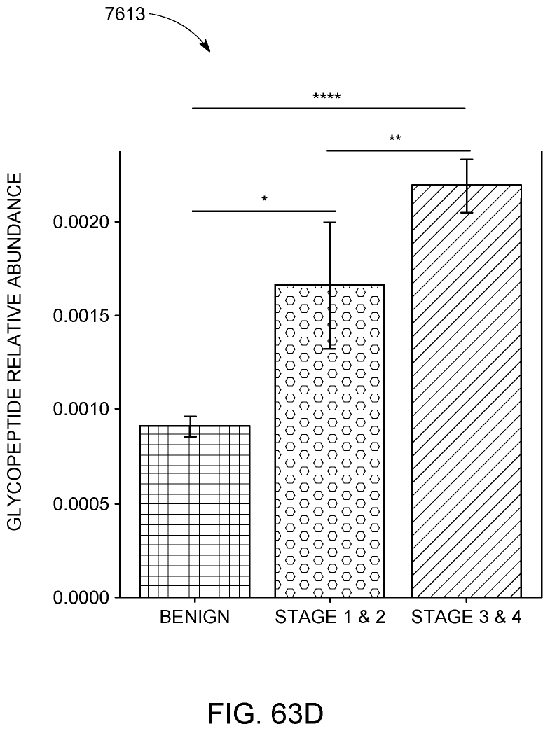

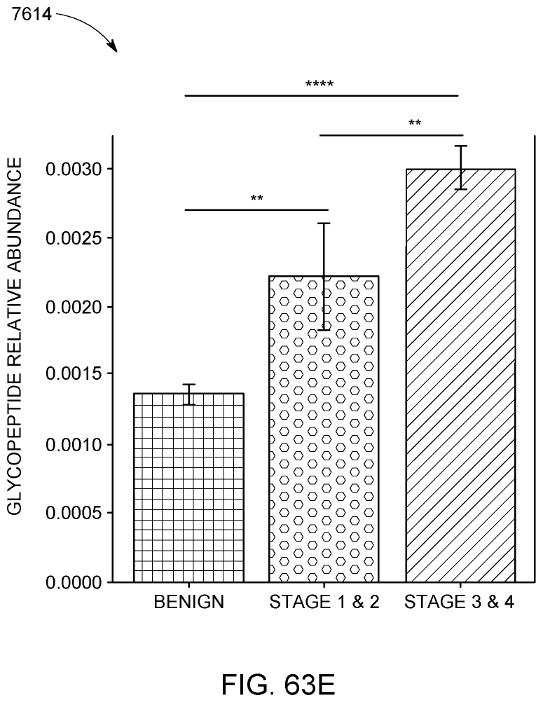

Using the model coefficients of Table 51C, a predicted probability was generated for early stage and late stage ovarian cancer samples showing a stratification in predicted probabilities between the two cohorts as is illustrated in FIG. 62. In various embodiments, predicted probability can be generated for classifying early stage and late stage ovarian cancer samples using the markers with non-zero coefficients such as SEQ ID NO's 504-509, 511, 513, 514, 517, 522, 523, 529, 532-536, 540, and 545. A logistic regression model was used with the glycopeptides of Table 43D where the glycopeptides had 1 or more sialic acids and zero or more fucosylations for the early and late stage EOC cohorts. The glycopeptides that included fucose were found to be associated with EOC. In addition, glycopeptides that included fucose and also carrying tri- and tetra-antennary glycan structure were found to be more strongly associated with EOC. For example, FIGS. 63A to 63E show that the relative abundance of tri- and tetra-antennary glycan structures in benign tumors, early-stage EOC and late-stage EOC showed an increase with the progression of the EOC disease. The numbers 6512, 6512, 7612, 7613, 7614 correspond to the five distinct glycans attached to the glycopeptides. Referring back to FIGS. 63A to 63C, the three leftmost bar graphs represent glycopeptides with tetra-antennary glycans with varying degrees of sialylation. The two rightmost bar graphs are FIGS. 63D and 63E and they represent glycopeptides with tri-antennary glycans with two or three sialic acids. The asterisk(s) in FIGS. 63A and 63E represent the following—* p-value<=0.05, ** p-value<=0.01, *** p-value<=0.001, and **** p-value<=0.0001). It should be noted that the horizontal bars on FIGS. 63A to 63E are used to show the statistical comparisons between the benign and late-stage cohorts (highest horizontal bar), early-stage and late-stage cohorts (middle horizontal bar), and the benign and early-stage cohorts (lowest horizontal bar),

Table 54 shows the accuracy, sensitivity, and specificity for the training data set and the testing data set.

TABLE 54

| | Train | Test |
|---|---|---|
| Accuracy | 0.963 | 0.8 |
| Sensitivity | 1 | 0.824 |
| Specificity | 0.812 | 0.667 |

### I.B. Exemplary Retrospective & Prospective Analysis

A validation study was conducted using both retrospective patient samples and samples collected prospectively in the ongoing Clinical Validation of the InterVenn Ovarian CAncer Liquid Biopsy (VOCAL) study. Samples included those from patients with malignant EOC and patients with benign pelvic tumors. Samples were processed in a manner similar to the manner described for the Exemplary Retrospective Analysis.

A logistic regression model was built identifying a panel of 38 peptide structures (same as those in Table 43A above). This panel of 38 peptide structures had an overall predictive accuracy of over 86% for the prediction of malignancy versus benign status of pelvic tumors.

Table 50A below provides the fold changes and p-values for the 38 peptide structures also identified in Table 43A above based on differential expression analysis (DEA). These peptide structures are ordered both in Table 43A and in Table 50A with respect to relative significance to the probability score generated by the model based on p-values. In this context, more significant peptide structures have lower p-values, while less significant peptide structures have higher p-values. In other words, relative significance to the probability score decreased with increasing p-values.

### II. Representative Experimental Results—Melanoma

### Samples:

Sample data via glycoproteomic analysis of pretreatment blood samples was compiled for a sample population comprising advanced malignant melanoma patients treated with pembrolizumab (Pembro; n=24) or nivolumab-ipilimumab (ipi/nivo; n=11). Samples were analyzed using an advanced glycoproteomics platform that combines ultra-high-performance liquid chromatography coupled to triple quadrupole mass spectrometry and a neural-network-based data processing engine. Individual glycopeptide signatures derived from 67 abundant serum proteins were analyzed and correlated with treatment, progression-free survival (PFS, and other clinical outcome metrics).

### Analysis:

Two response groups were defined based on PFS: early disruption (e.g., early failure) (EF; PFS event within 6 months) and sustained control (SC; no events for ≥12 months). Differential relative/absolute abundances for 498 serum glycopeptides and aglycosylated peptides were calculated between SC and EF patients for each treatment group to determine a set of peptide structures more abundant in each SC versus EF by treatment group. A score was developed for each treatment group based on the 20 markers within each treatment group identified as the most statistically significant ones based on one-sided Wilcoxon test comparing EF and SC. For a given patient, the score was computed as the proportion of glycopeptides/aglycosylated peptides with relative/absolute abundance exceeding their median abundance. A low score was associated with high risk for early failure.

Table 64 and Table 65 below show the median abundances identified for the set of peptide structures. These median abundances are examples of what may be used as reference abundances for these peptide structures.

TABLE 64
Median Abundances for Peptide Structures associated with Pembro

| | Peptide | |
|---|---|---|
| PS- | Structure | Median |
| ID NO. | (PS) NAME | Abundance |
| 330 | IGG1_297_5400 | 0.1044605 |
| 331 | IGG2_297_5411 | 0.1214551 |
| 332 | IGG1_297_5510 | 0.1032259 |
| 333 | IGG2_297_5410 | 0.0993292 |
| 334 | IGG1_297_5410 | 0.0525704 |
| 335 | IGG2_297_4411 | −0.0737209 |
| 336 | THBG_36_5402 | 0.1421563 |
| 337 | IGG2_297_5510 | 0.1248705 |
| 338 | AGP1_33_6503 | 0.3600423 |
| 339 | CO8B_243_6610 | 0.1909322 |
| 340 | IGA12_144_5502 | −0.1191828 |
| 341 | KLKB1_494_5410 | 0.0492207 |
| 342 | IGG1_297_4400 | 0.1883168 |
| 343 | AACT_271_7602 | 0.0923715 |
| 344 | CO8B_553_5410 | −0.014819 |
| 345 | FETUA_156_5402.5421 | −0.2216863 |
| 346 | IGA12_144_5501 | 0.0029389 |
| 347 | IGG2_297_4500 | 0.0712961 |
| 348 | AGP1_33_6502 | 0.0921509 |
| 349 | CLUS_374_6520.6501 | 0.0556276 |

TABLE 65
Median Abundances for Peptide Structures
associated with Ipi/Nivo

| PS- | Peptide | |
|---|---|---|
| ID | Structure | Median |
| NO. | (PS) NAME | Abundance |
| 350 | A2MG_869_5200 | 0.0754619 |
| 338 | AGP1_33_6503 | 0.3600423 |
| 351 | CFAH_882_5420.5401 | 0.1460826 |
| 352 | CFAH_911_5420.5401 | 0.1281516 |
| 353 | HEMO_453_5420.5401 | 0.2013525 |
| 354 | IGG34_297_4410 | 0.2134462 |
| 355 | KLKB1_127_5410 | −0.0022041 |
| 356 | TRFE_432_5401 | −0.0482695 |
| 357 | QUANTPEP.IGG4_ | 0.0439244 |
| | TTPPVLDSDGSFFLYSR | |
| 358 | NEWQUANTPEP- | −0.0280153 |
| | IGG3_ | |
| | TPEVTCVVVDVSHEDPEVQFK | |
| 359 | A2MG_869_6200 | −0.0430135 |
| 360 | HPT_184_5511 | −0.2843536 |
| 361 | VTNC_169_5401 | −0.0248306 |
| 362 | AACT_271_7603 | 0.1237403 |
| 363 | HPT_207_10803 | 0.0825476 |
| 364 | HPT_241_5401.5420 | −0.0008547 |
| 365 | IGG34_297_4411 | 0.2102183 |
| 366 | ITIH4_517_5420.5401 | 0.0750044 |
| 341 | KLKB1_494_5410 | 0.0492207 |
| 367 | AACT_127_5401 | 0.0715 |

### Results:

When examined in all patients in the cohort (regardless of treatment), both treatment scores isolated EF from SC. Algorithmic assignment was performed by choosing the treatment with the highest treatment-specific score (e.g., if ipi/nivo score >pembro score, then assign to ipi/nivo). PFS was superior for cases where the assigned treatment matched the treatment received. Log-rank p-values comparing PFS by assigned treatment within pembro- and ipi/nivo-treated cases were 0.009 and 0.0004, respectively. Our results show that serum glycoproteomic analysis allows targeted treatment assignment not only to immune checkpoint inhibitor treatment in general, but specifically to the most likely successful agent among different drugs for melanoma. This may fundamentally improve the clinical use of immuno-therapy in subjects with melanoma.

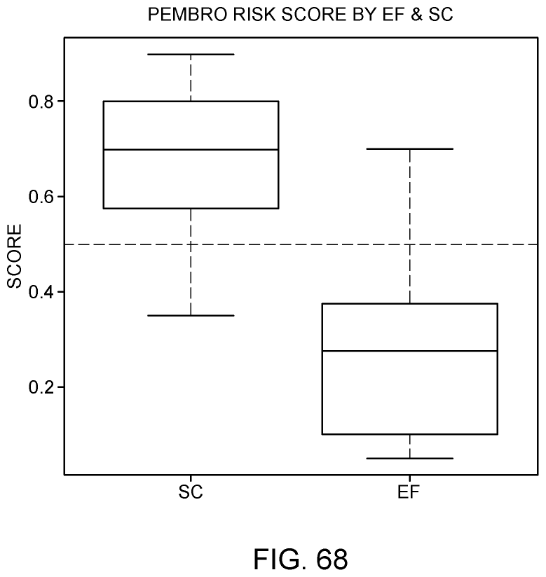

FIG. 68 is a plot showing the distribution of the treatment scores generated for those patients who were treated with pembro in accordance with one or more embodiments.

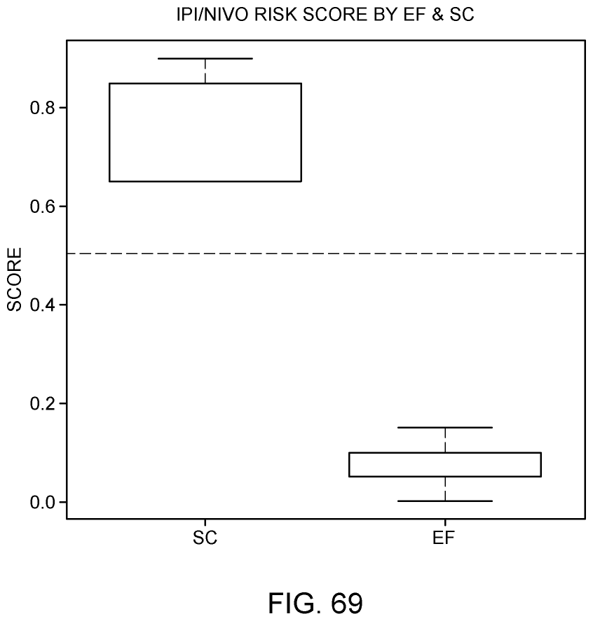

FIG. 69 is a plot showing the distribution of the treatment scores generated for those patients who were treated with ipi/nivo in accordance with one or more embodiments.

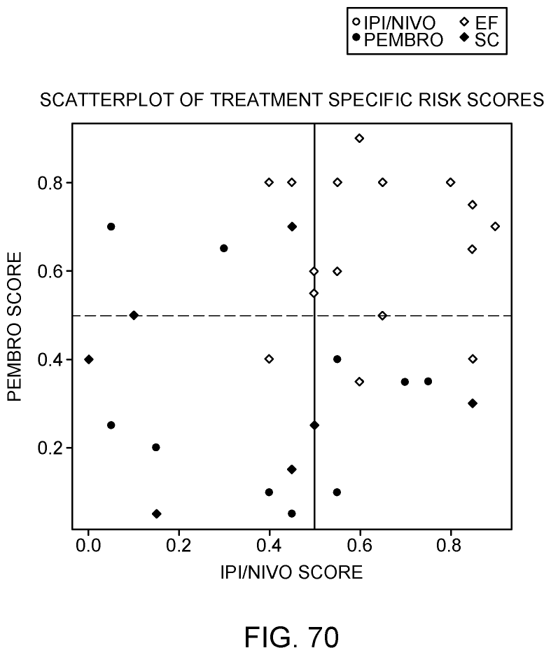

FIG. 70 is a scatterplot showing the treatment scores by treatment type in accordance with one or more embodiments.

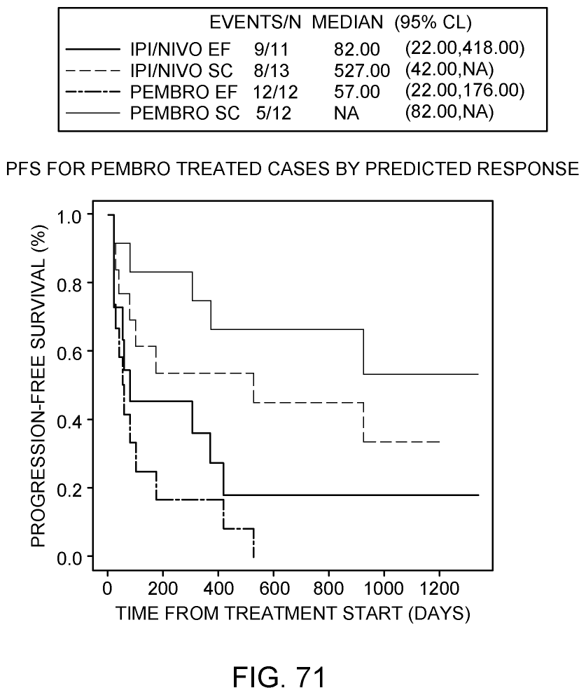

FIG. 71 is a plot showing disruption event times for patients treated with pembro by their predicted response.

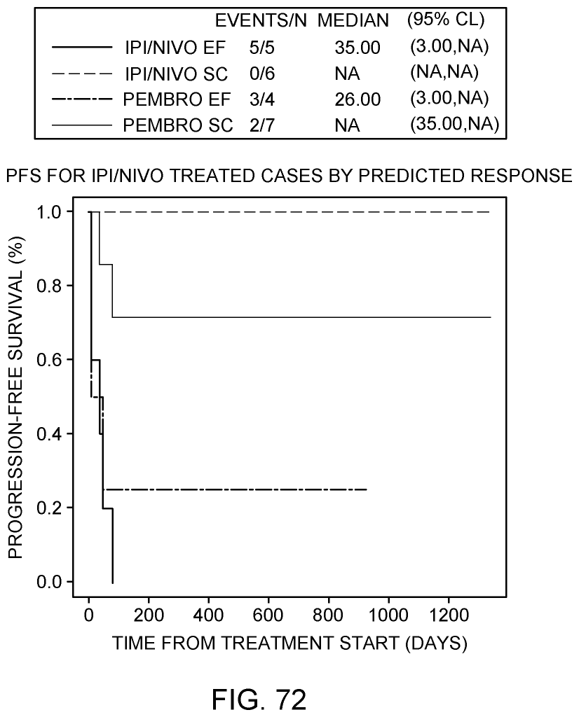

FIG. 72 is a plot showing disruption event times for patients treated with ipi/nivo by their predicted response.

### 2. Biomarkers for Determining Immuno-Oncologyresponse

Provided herein are methods, devices, glycopeptides, and kits for identifying glycoproteomic biomarkers and signatures for risk of having a disease or a condition, progression of the disease or condition, and response of the disease or condition to a treatment, such as treatment with immune checkpoint blockade for cancer. In some cases, the disease or condition may be cancer. In some cases, the progression of the disease or condition includes but is not limited to stage of cancer or size of tumor or a surrogate endpoint. Such information may be used to provide actionable recommendations for treatment to a healthcare provider, including but not limited to initiation of a new treatment, continuation of ongoing treatment, adding a new therapy, or changing the dosage and/or frequency of ongoing treatment.

Protein glycosylation is one of the abundant and most complex form of post-translational protein modification. Glycosylation profoundly can affect structure, conformation, and function of a polypeptide. The elucidation of the potential role of differential polypeptide glycosylation as biomarkers has so far been limited by the technical complexity of generating and interpreting this information. A novel, powerful platform has been established that combines ultra-high-performance liquid chromatography (LC) coupled to triple quadrupole mass spectrometry (MS) with a machine-learning and neural-network-based data processing engine that allows for high-throughput, highly scalable interrogation of the glycoproteome. The glycoproteomic biomarkers and signatures may be used to predict which cancer patients may respond to immune checkpoint blockade treatment, such as PD1/PDL1 checkpoint inhibitors.

Changes in glycosylation have been described in relationship to disease states such as cancer. See, e.g., Dube, D. H.; Bertozzi, C. R. Glycans in Cancer and Inflammation—Potential for Therapeutics and Diagnostics. Nature Rev. Drug Disc. 2005, 4, 477-88, the entire contents of which are herein incorporated by reference in its entirety for all purposes. However, clinically relevant, non-invasive assays for diagnosing cancer in a patient based on glycosylation changes in a sample from that patient are still needed.

Mass spectroscopy (MS) offers sensitive and precise measurement of cancer-specific biomarkers including glycopeptides. See, for example, Ruhaak, L. R., et al., Protein-Specific Differential Glycosylation of Immunoglobulins in Serum of Ovarian Cancer Patients DOI: 10.1021/acs.jproteome.5b01071; J. Proteome Res., 2016, 15, 1002-1010 (2016); also Miyamoto, S., et al., Multiple Reaction Monitoring for the Quantitation of Serum Protein Glycosylation Profiles: Application to Ovarian Cancer, DOI: 10.1021/acs.jproteome.7b00541, J. Proteome Res. 2018, 17, 222-233 (2017), the entire contents of which are herein incorporated by reference in its entirety for all purposes. However, using MS to diagnose cancer has not been demonstrated to date in a clinically relevant manner. What is needed are new biomarkers and new methods of using MS to assess a diagnosis for a disease or a condition, a risk of having a disease or a condition, progression of the disease or condition, and response of the disease or condition to a treatment.

### I. Overview

Provided herein are methods for identifying one or more glycopeptide biomarkers predictive of a disease or a condition in a subject, the method comprising: (a) obtaining from a subject a first sample at a first timepoint and a second sample at a second timepoint, wherein the first sample and the second sample comprise a glycoprotein; (b) fragmenting the glycoprotein in the first sample or the second sample into one or more glycopeptides, wherein the one or more glycopeptides comprise one or more amino acid sequences selected from a group consisting of SEQ ID NO: 673-703, 731-779, and 570-595, and combinations thereof, (c) determining an amount of the one or more glycopeptides using multiple reaction monitoring mass spectrometry (MRM-MS); (d) associating the amount of the one or more glycopeptides with the first timepoint or the second timepoint, wherein the subject has a change in a disease or a condition from the first timepoint to the second timepoint; and (e) identifying as glycopeptide biomarkers the glycopeptide where the amount of the one or more glycopeptides changed from the first timepoint to the second timepoint.

Described herein are methods for identifying one or more glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) assessing, by the computer, the predictive model using a cross-validation with n-1 subjects to generate an outcome score for a holdout subject; (d) iterating, by the computer, step (c) for each of n subjects as the holdout subject to generate an outcome score for each subject; (e) dichotomizing, by the computer, the outcome scores for each subject at a cutoff outcome score as below or above the cutoff outcome score; (f) analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide; (g) identifying, by the computer, the glycopeptide having the interaction p-value <0.05 as a glycopeptide biomarker for predicting the disease or the condition. In some embodiments, the cross-validation is leave-one-out cross-validation (LOOCV). In some embodiments, the cutoff outcome score was determined to optimize Harrell's C-index. In some embodiments, the interaction p-value is less than or equal to 0.01, 0.005, or 0.001 in step (g).

Provided herein are method for identifying one or more glycopeptide biomarkers predictive of a disease or a condition in a subject, the method comprising: (a) obtaining, by a computer, data of an amount of one or more glycopeptides for a set (n) of subjects, wherein the one or more glycopeptides are generated by fragmenting a glycoprotein in a sample from a subject, the amount of one or more glycopeptides are determined using multiple reaction monitoring mass spectrometry (MRM-MS), and the data for each subject comprises data from samples taken at a plurality of timepoints; (b) selecting, by the computer, a subset of the one or more glycopeptides to include in a predictive model; (c) assessing, by the computer, the predictive model using a cross-validation with n-1 subjects to generate an outcome score for a holdout subject; (d) iterating, by the computer, step (c) for each of n subjects as the holdout subject to generate an outcome score for each subject; (e) dichotomizing, by the computer, the outcome scores for each subject at a cutoff outcome score as below or above the cutoff outcome score; (f) analyzing, by the computer, the amount of one or more glycopeptides for subjects having outcome scores above the cutoff outcome score to the amount of one or more glycopeptides for subjects having outcome scores below the cutoff outcome score for each glycopeptide in the subset of the one or more glycopeptides to determine a hazard ratio and an interaction p-value for each glycopeptide; (g) identifying, by the computer, the glycopeptide having the interaction p-value ≤0.05 as a glycopeptide biomarker for predicting the disease or the condition.

Described herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 673-703, 731-779, and 570-595, and combinations thereof, (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is melanoma and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

Provided herein are methods for assessing a status of a condition and a treatment in a subject, the method comprising: (a) fragmenting a glycoprotein in a sample from a subject into one or more glycopeptides, wherein the sample comprises one or more of glycoproteins, glycans, or glycopeptides; (b) performing mass spectroscopy (MS) on the one or more glycopeptides using multiple reaction monitoring mass spectrometry (MRM-MS) to quantify an amount of the one or more glycopeptides in the sample, wherein the one or more glycopeptides comprise one or more amino acid sequences selected from a group consisting of SEQ ID NOs: 673-703, 731-779, and 570-595, and combinations thereof, (c) inputting data of the amount of the one or more glycopeptides into a trained model to generate an output probability, wherein the output probability is indicative of whether a treatment positively influences an outcome of the subject having a condition; and (d) generating a treatment recommendation based on the output probability, wherein the condition is non-small cell lung cancer (NSCLC) and the treatment comprises checkpoint inhibitors. In some embodiments, the outcome comprises overall survival time. In some embodiments, the outcome comprises progression-free survival time. In some embodiments, the treatment comprises one or more of ipilimumab, nivolumab, and pembrolizumab. In some embodiments, the treatment comprises one or more of PD-1-, PD-L1-, and CTLA-4-inhibitors. In some embodiments, the treatment comprises chemotherapy. In some embodiments, the chemotherapy comprises one or more of carboplatin and pemetrexed. In some embodiments, the recommendation comprises continuing the treatment if the output probability indicates the treatment positively influences the outcome.

In some embodiments, provided herein are methods for identifying a classification for a sample, the method comprising: quantifying by mass spectroscopy (MS) one or more glycopeptides in a sample wherein the glycopeptides each, individually in each instance, comprises a glycopeptide consisting essentially of an amino acid sequence selected from the group consisting of SEQ ID NO: 673-703, 731-779, and 570-595, and combinations thereof, and inputting the quantification into a trained model to generate an output probability; determining if the output probability is above or below a threshold for a classification; and identifying a classification for the sample based on whether the output probability is above or below a threshold for a classification.

In some embodiments, provided herein are methods for training a machine-learning algorithm, comprising: providing a first data set of MRM transition signals indicative of a sample comprising a glycopeptide consisting of, or consisting essentially of, an amino acid sequence selected from the group consisting of SEQ ID NO: 673-703, 731-779, and 570-595; providing a second data set of MRM transition signals indicative of a control sample; and comparing the first data set with the second data set using a machine-learning algorithm.

In some embodiments, provided herein are methods for diagnosing a patient having cancer; the method comprising: obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NO: 673-703, 731-779, and 570-595; or to detect and quantify one or more MRM transitions; inputting the quantification of the detected glycopeptides or the MRM transitions into a trained model to generate an output probability, determining if the output probability is above or below a threshold for a classification; identifying a diagnostic classification for the patient based on whether the output probability is above or below a threshold for a classification; and providing a recommendation for treatment. In some examples, the method includes performing mass spectroscopy of the biological sample using MRM-MS with a QQQ.

### II. Biomarkers

Provided herein are glycopeptide biomarkers. These biomarkers are useful for a variety of applications, including, but not limited to, diagnosing diseases and conditions. For example, certain biomarkers set forth herein, or combinations thereof, are useful for diagnosing cancer. In some embodiments, the cancer is melanoma. In some embodiments, the cancer is non-small cell lung cancer (NSCLC). In some embodiments, the biomarkers are useful for diagnosing and screening patients having cancer, an autoimmune disease, or fibrosis. In some embodiments, the biomarkers are useful for classifying a patient so that the patient receives the appropriate medical treatment. In some embodiments, the biomarkers are useful for treating or ameliorating a disease or condition in patient by, for example, identifying a therapeutic agent with which to treat a patient. In some embodiments, the biomarkers are useful for determining a prognosis of treatment for a patient or a likelihood of success or survivability for a treatment regimen.

In some embodiments, a sample from a patient is analyzed by MS and the results are used to determine the presence, absolute amount, and/or relative amount of a glycopeptide consisting of an amino acid sequence selected from SEQ ID NO: 673-703, 731-779, and 570-595 in the sample. In some embodiments, a sample from a patient is analyzed by MS and the results are used to determine the presence, absolute amount, and/or relative amount of a glycopeptide consisting essentially of an amino acid sequence selected from SEQ ID NO: 673-703, 731-779, and 570-595 in the sample. In some embodiments, a sample from a patient is analyzed by MS and the results are used to determine the presence, absolute amount, and/or relative amount of a glycopeptide consisting of, or consisting essentially of, an amino acid sequence selected from SEQ ID NO: 673-703, 731-779, and 570-595 in the sample. In some embodiments, a sample from a patient is analyzed by MS and the results are used to determine the presence, absolute amount, and/or relative amount of a glycopeptide consisting of, or consisting essentially of, an amino acid sequence selected from SEQ ID NO: 673-703, 731-779, and 570-595 in the sample. In some embodiments, the presence, absolute amount, and/or relative amount of a glycopeptide is determined by analyzing the MS results. In some embodiments, the MS results are analyzed using machine-learning.

Provided herein are biomarkers selected from glycans, peptides, glycopeptides, fragments thereof, and combinations thereof. In some embodiments, the glycopeptide comprise an amino acid sequence selected from SEQ ID NO: 673-703, 731-779, and 570-595. In some embodiments, the glycopeptide consists essentially of an amino acid sequence selected from SEQ ID NO: 673-703, 731-779, and 570-595.

### O-Glycosylation

In some examples, the glycopeptides set forth herein include O-glycosylated peptides. These peptides include glycopeptides in which a glycan is bonded to the peptide through an oxygen atom of an amino acid. Typically, the amino acid to which the glycan is bonded is threonine (T) or serine (S). In some examples, the amino acid to which the glycan is bonded is threonine (T). In some examples, the amino acid to which the glycan is bonded is serine (S).

In certain examples, the O-glycosylated peptides include those peptides from the group selected from Apolipoprotein C-III (APOC3), Alpha-2-HS-glycoprotein (FETUA), and combinations thereof. In certain examples, the O-glycosylated peptide, set forth herein, is an Apolipoprotein C-III (APOC3) peptide. In certain examples, the O-glycosylated peptide, set forth herein, is an Alpha-2-HS-glycoprotein (FETUA).

### N-Glycosylation

In some examples, the glycopeptides set forth herein include N-glycosylated peptides. These peptides include glycopeptides in which a glycan is bonded to the peptide through a nitrogen atom of an amino acid. Typically, the amino acid to which the glycan is bonded is asparagine (N) or arginine (R). In some examples, the amino acid to which the glycan is bonded is asparagine (N). In some examples, the amino acid to which the glycan is bonded is arginine (R).

In certain examples, the N-glycosylated peptides include members selected from the group consisting of Alpha-1-antitrypsin (AlAT), Alpha-iB-glycoprotein (A1BG), Leucine-richAlpha-2-glycoprotein (A2GL), Alpha-2-macroglobulin (A2MG), Alpha-1-antichymotrypsin (AACT), Afamin (AFAM), Alpha-1-acid glycoprotein 1 & 2 (AGP12), Alpha-1-acid glycoprotein 1 (AGP1), Alpha-1-acid glycoprotein 2 (AGP2), Apolipoprotein A-1 (APOA1), Apolipoprotein B-100 (APOB), Apolipoprotein D (APOD), Beta-2-glycoprotein-1 (APOH), Apolipoprotein M (APOM), Attractin (ATRN), Calpain-3 (CAN3), Ceruloplasmin (CERU), Complement Factor H (CFAH), Complement Factor I (CFAI), Clusterin (CLUS), ComplementC3 (CO3), ComplementC4-A&B (CO4A&CO4B), ComplementcomponentC6 (CO6),

ComplementComponentC8AChain (CO8A), Coagulation factor XII (FA12),

Haptoglobin (HPT), Histidine-rich Glycoprotein (HRG), Immunoglobulin heavy constant alpha 1&2 (IgAl2), Immunoglobulin heavy constant alpha 2 (IgA2),

Immunoglobulin heavy constant gamma 2 (IgG2), Immunoglobulin heavy constant mu (IgM), Inter-alpha-trypsin inhibitor heavy chain H1 (ITIH1), Plasma Kallikrein (KLKB1),

Kininogen-1 (KNG1), Serum paraoxonase/arylesterase 1 (PON1), Selenoprotein P (SEPP1), Prothrombin (THRB), Serotransferrin (TRFE), Transthyretin (TTR), Protein unc-13HomologA (UN13A), Vitronectin (VTNC), Zinc-alpha-2-glycoprotein (ZA2G), Insulin-like growth factor-II (IGF2), Apolipoprotein C-1 (APOC1), Hemopexin (HEMO), Immunoglobulin heavy constant gamma 1 (IgG1), Immunoglobulin J chain (IgJ), and combinations thereof.

### Peptides and Glycopeptides

In some examples, set forth herein is a glycopeptide or peptide consisting of an amino acid sequence selected from the group consisting of SEQ ID NO: 673-703, 731-779, and 570-595, and combinations thereof.

In some examples, set forth herein is a glycopeptide or peptide consisting essentially of an amino acid sequence selected from the group consisting of SEQ ID NO: 673-703, 731-779, and 570-595, and combinations thereof.

### III. Methods

Provided herein are methods of identifying the glycoproteomic biomarkers and signatures that may be used to predict which cancer patients respond to immune checkpoint blockade treatment, such as PD1/PDL1 checkpoint inhibitors, and have an improvement or a positive change in their condition.

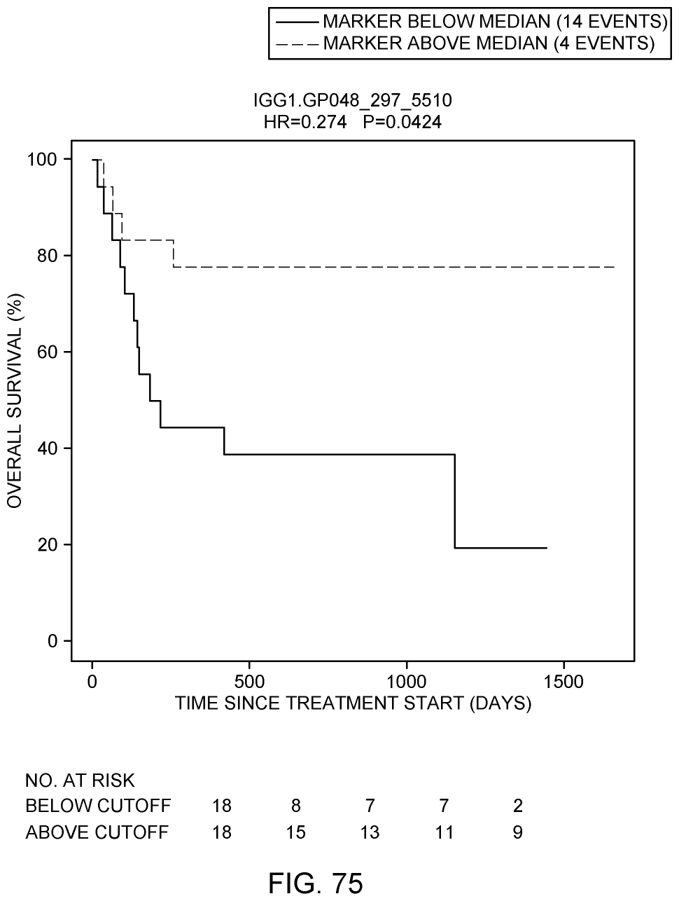

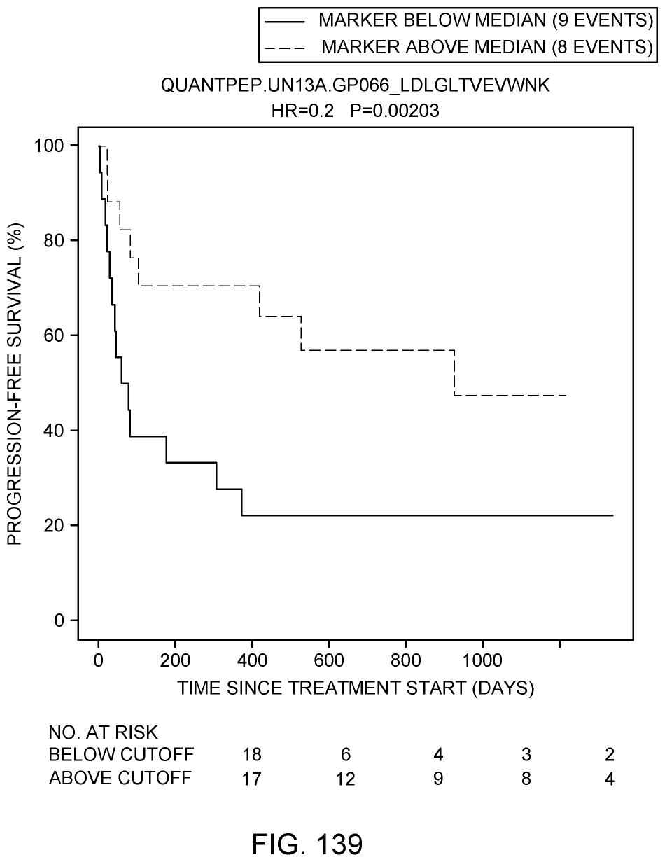

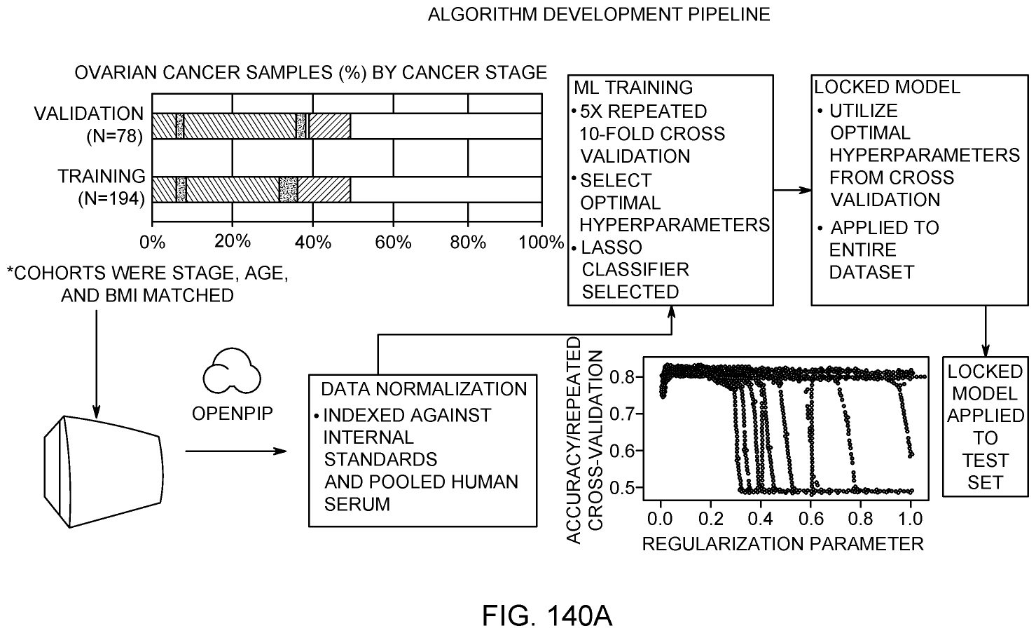

In some embodiments, individual glycopeptide expression levels are associated with various timepoints to determine which glycopeptides changed with events, such as death or metastasis, at the various timepoints. In some embodiments, individual glycopeptide expression levels are associated with time from treatment initiation to progression/metastasis (progression-free survival, PFS) or death (overall survival, OS) in the patient cohorts. In some embodiments, examples of individual glycopeptide expression levels are shown in FIGS. 75-139.

In some embodiments, multivariable models are used predict OS and PFS in cancer patients. In some embodiments, the cancer patients have NSCLC or melanoma. In some embodiments, a small subset of glycopeptides for modeling are selected, a model with n-1 patients from a total of n patients is built, a survival score on the one holdout patient is predicted, and the step are iterated over all patients as individual holdouts, to generate unbiased prediction scores for everyone (a leave-one-out cross-validation approach, LOOCV). In some embodiments, the resulting scores are dichotomized at a cutoff which optimizes Harrell's C-index. In some embodiments, Kaplan-Meier (KM) curves were plotted for each glycopeptide.

In some embodiments, hazard ratio (HR), p-value, and interaction P-value were calculated. In some embodiments, hazard ratio (HR) is calculated from a Cox Proportional Hazards model, representing the multiplicative increase in odds of death or progression-free survival time for each increase of the biomarker by 1 unit. In some embodiments, p-value is associated with the HR above. In some embodiments, P<0.01 was considered significant. In some embodiments, P≤0.05, P≤0.01, P≤0.005, or P≤0.001 was considered significant. In some embodiments, interaction P-value is associated with the biomarker ×treatment interaction; significance indicates potential for use in treatment selection.

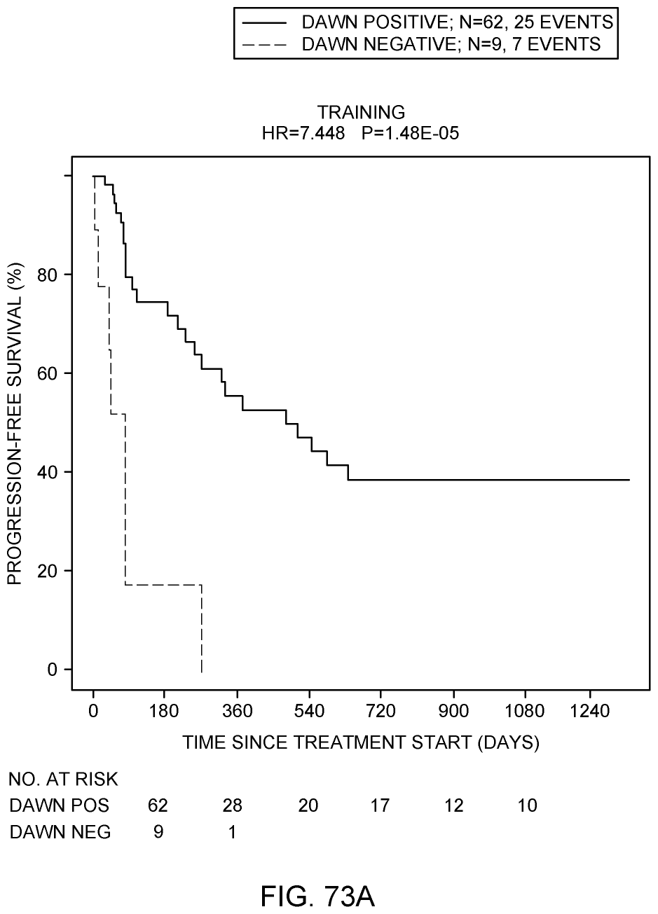

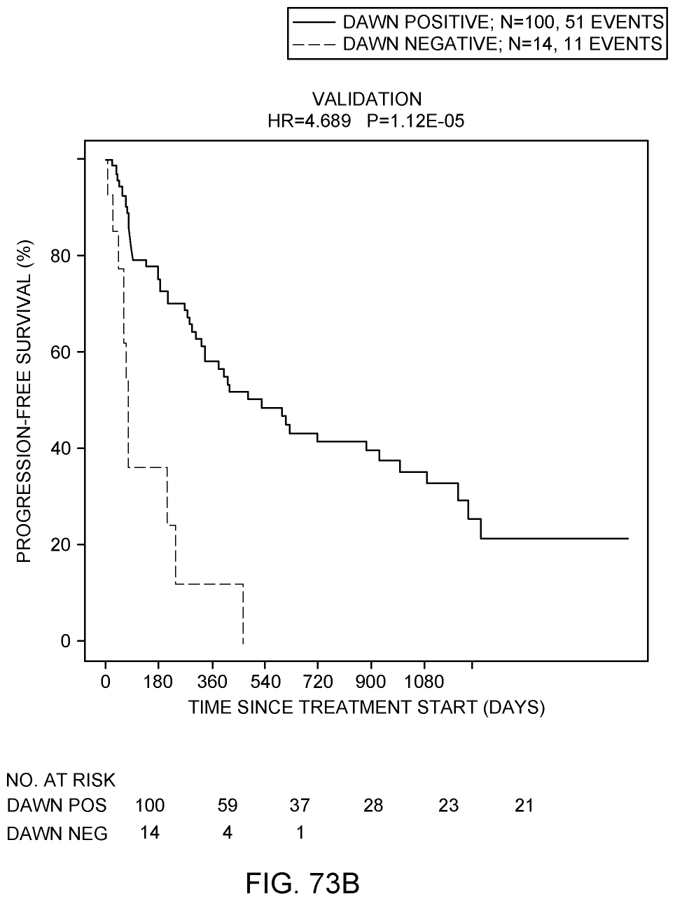

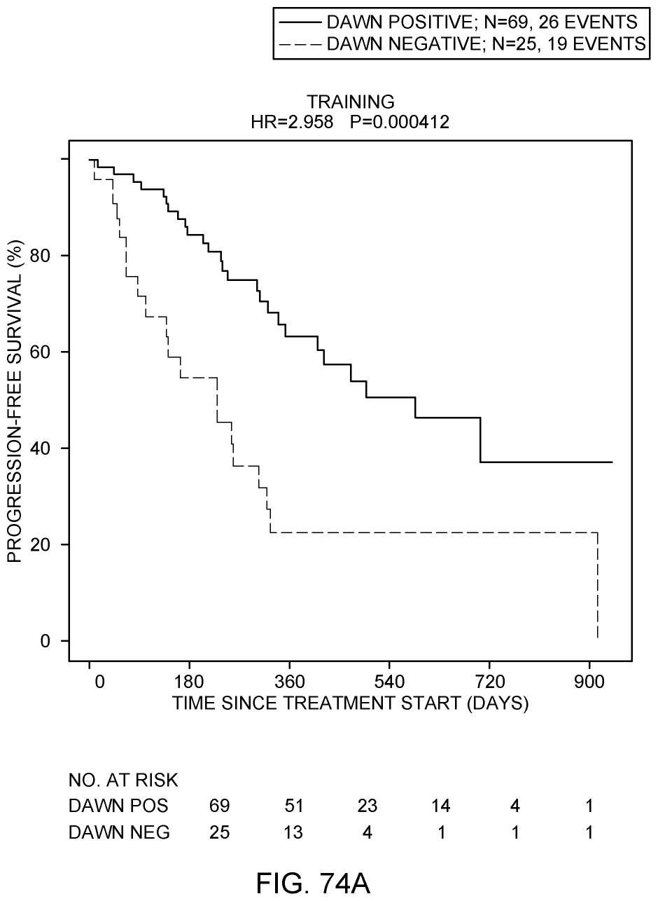

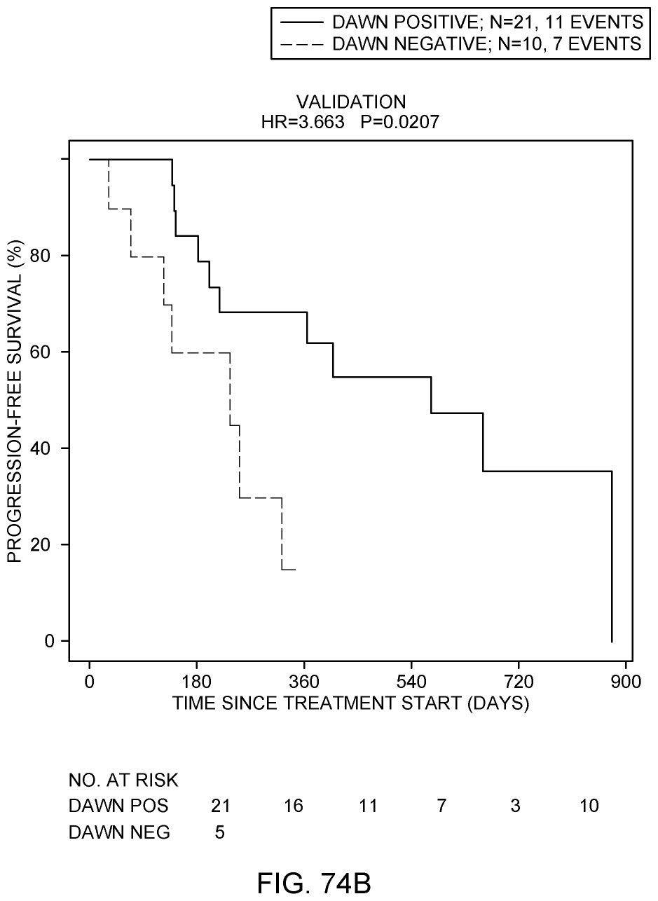

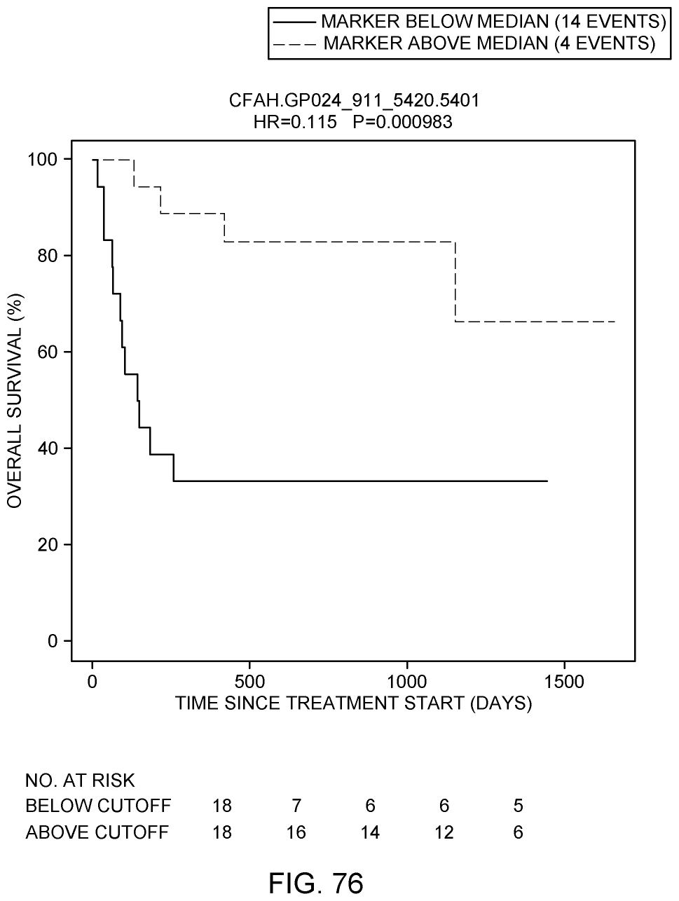

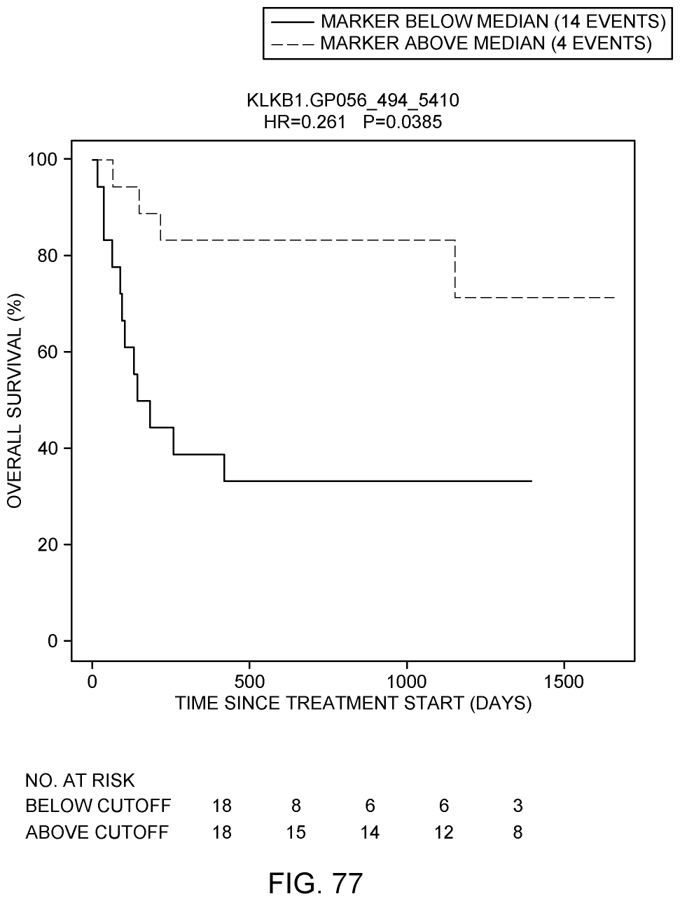

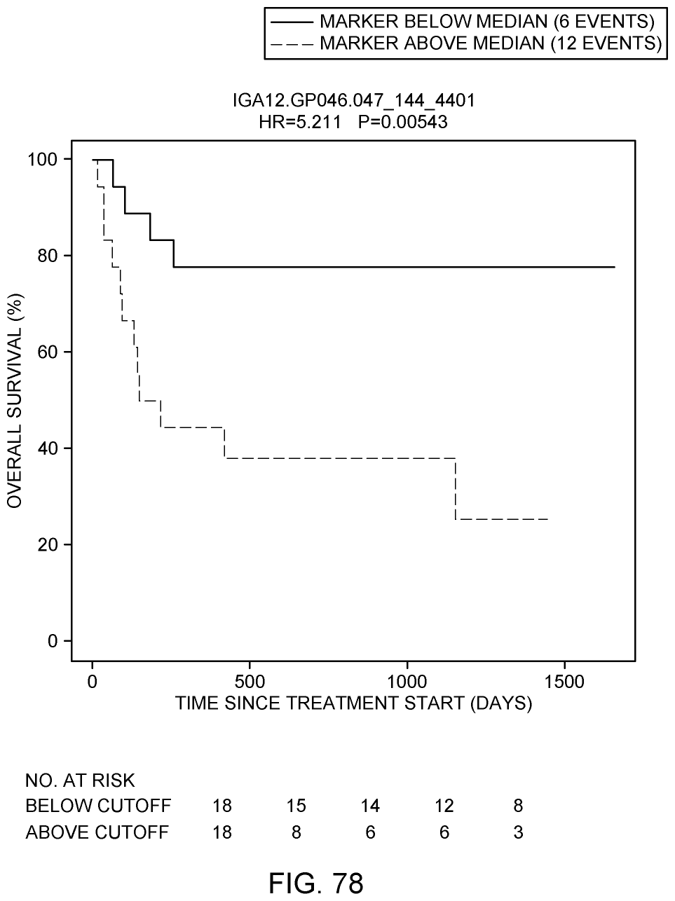

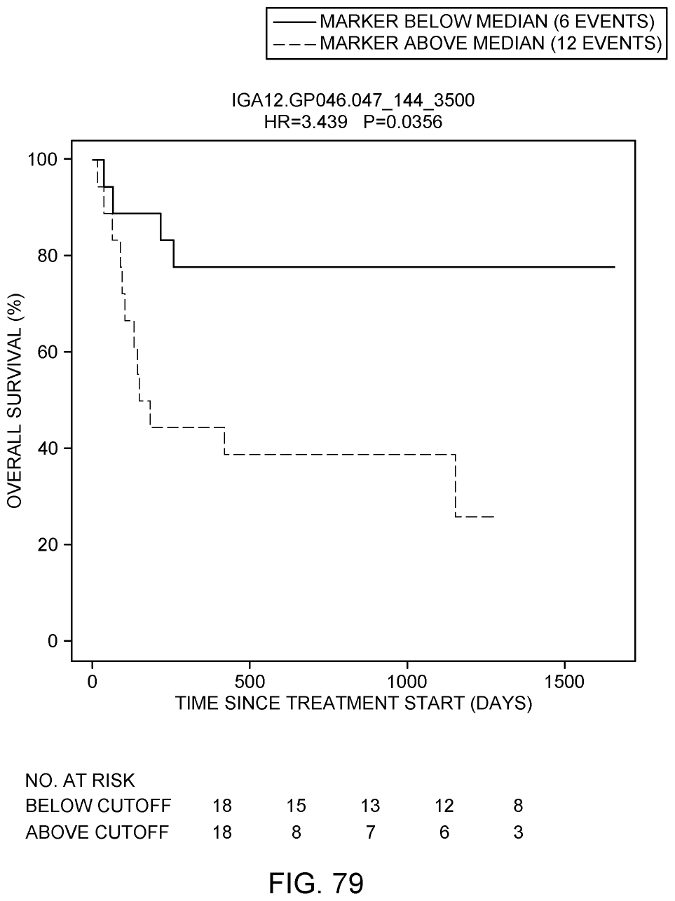

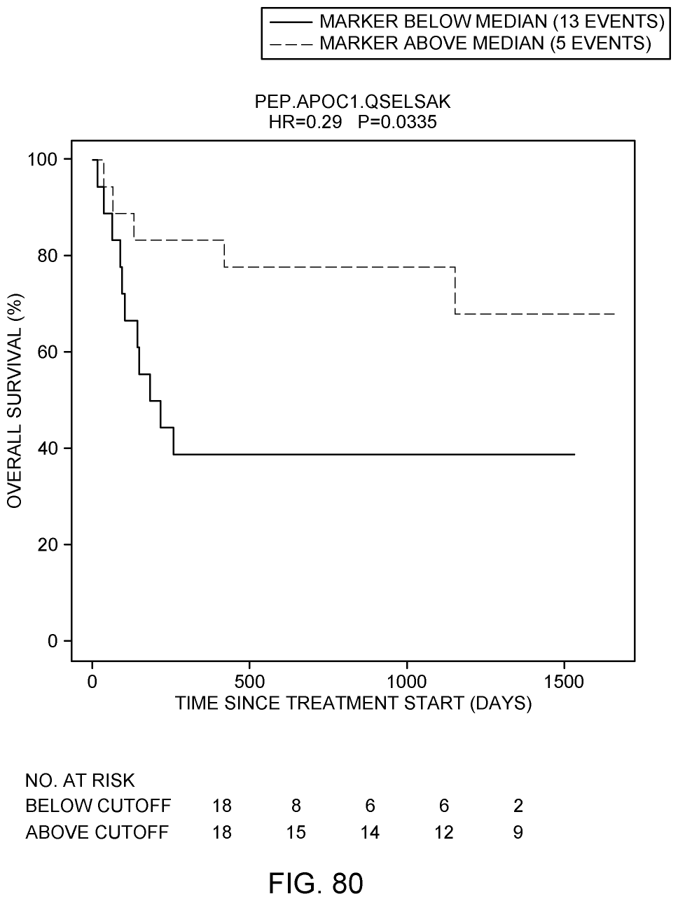

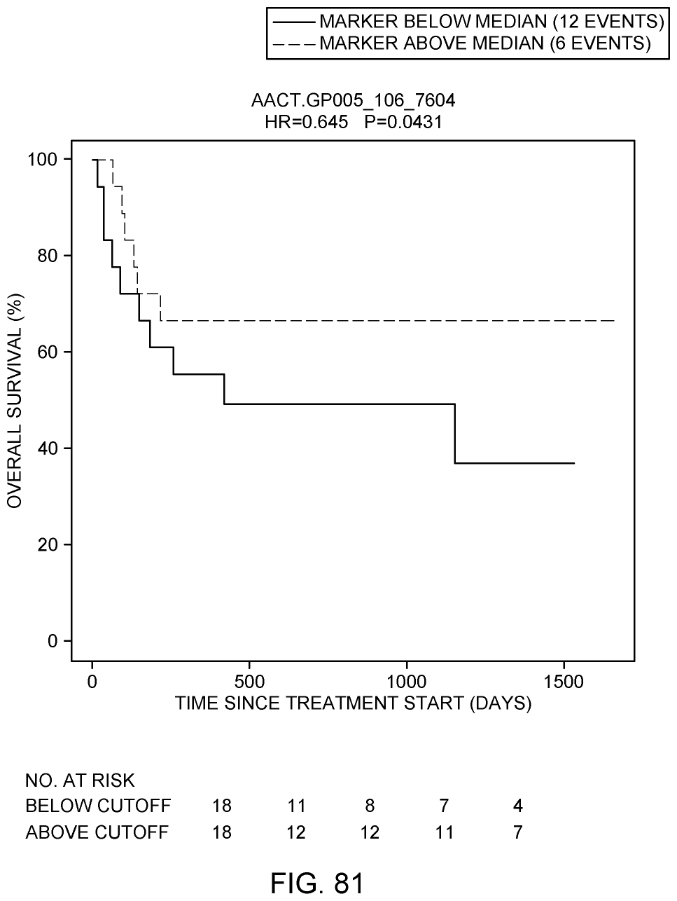

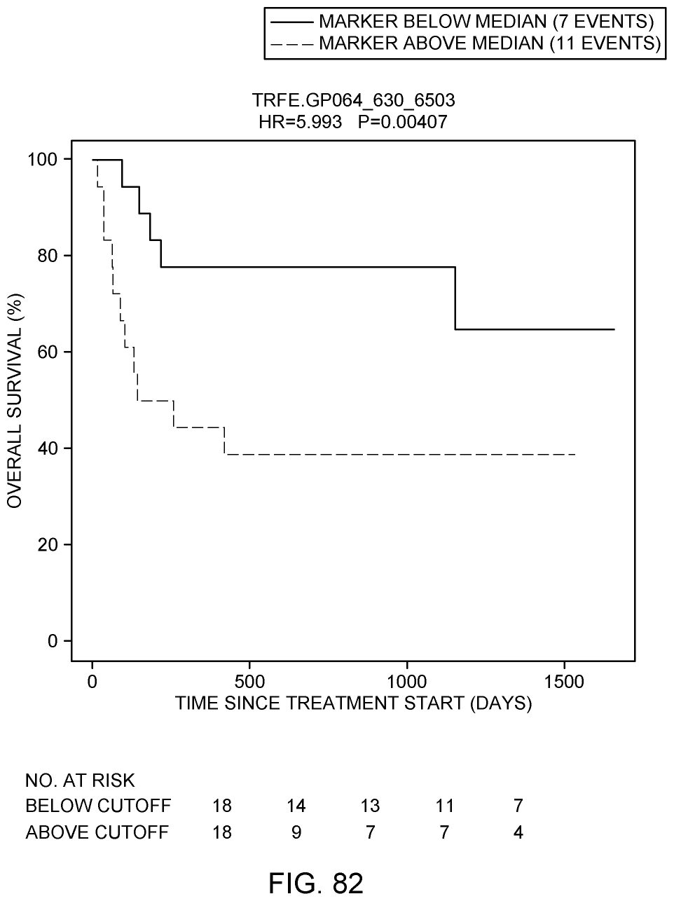

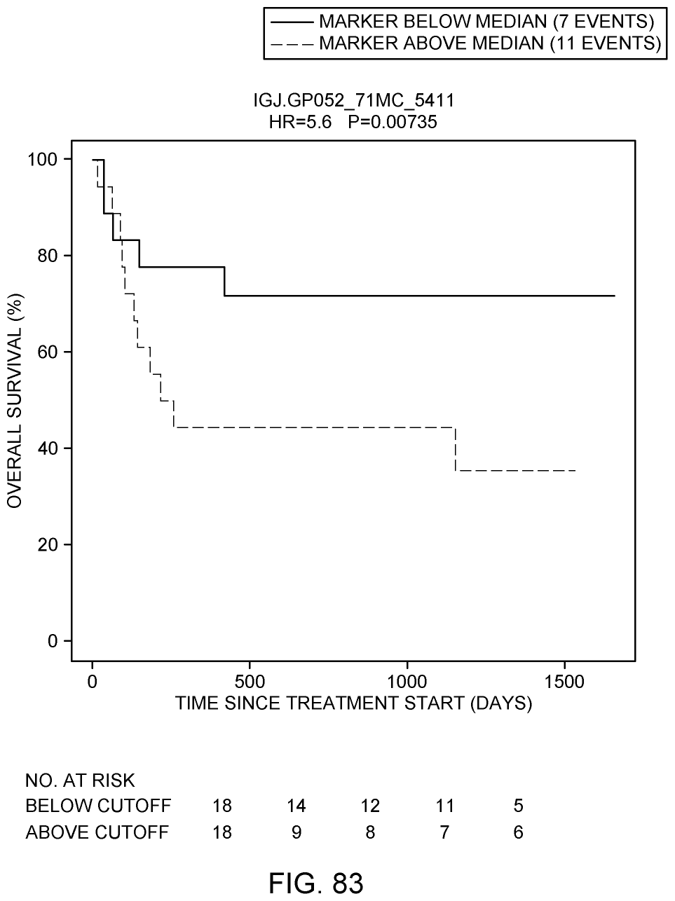

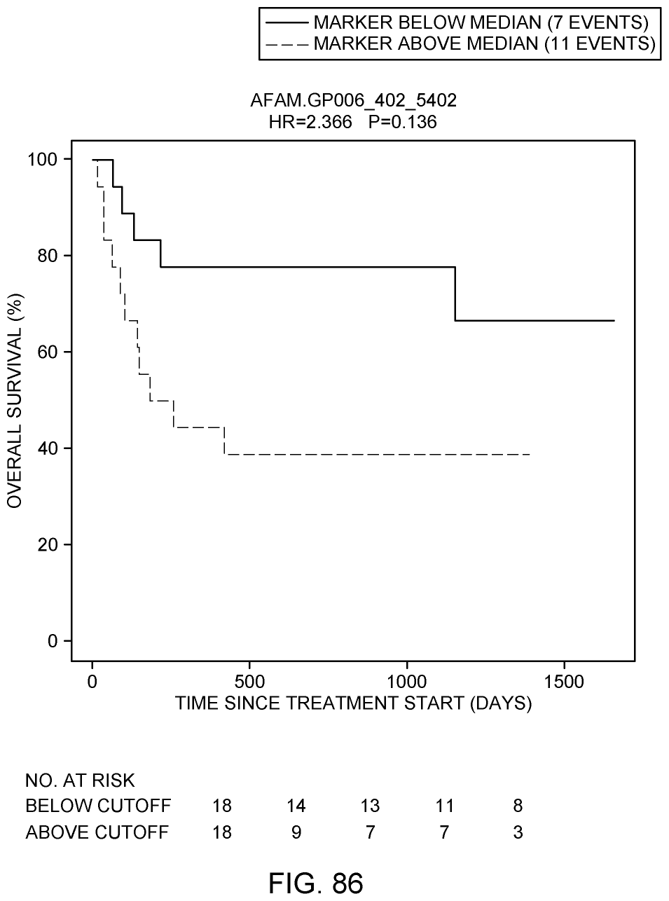

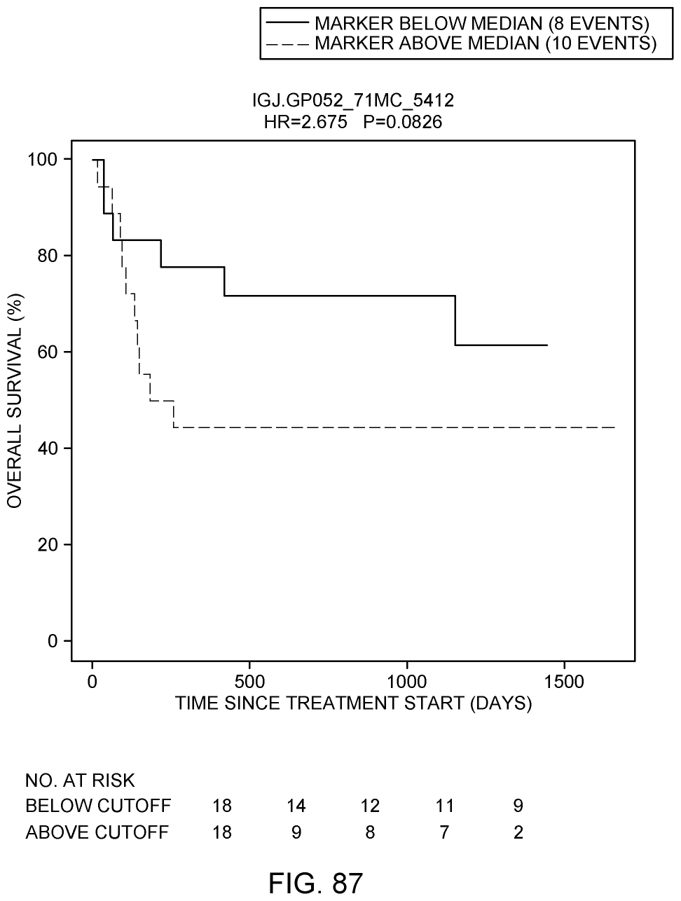

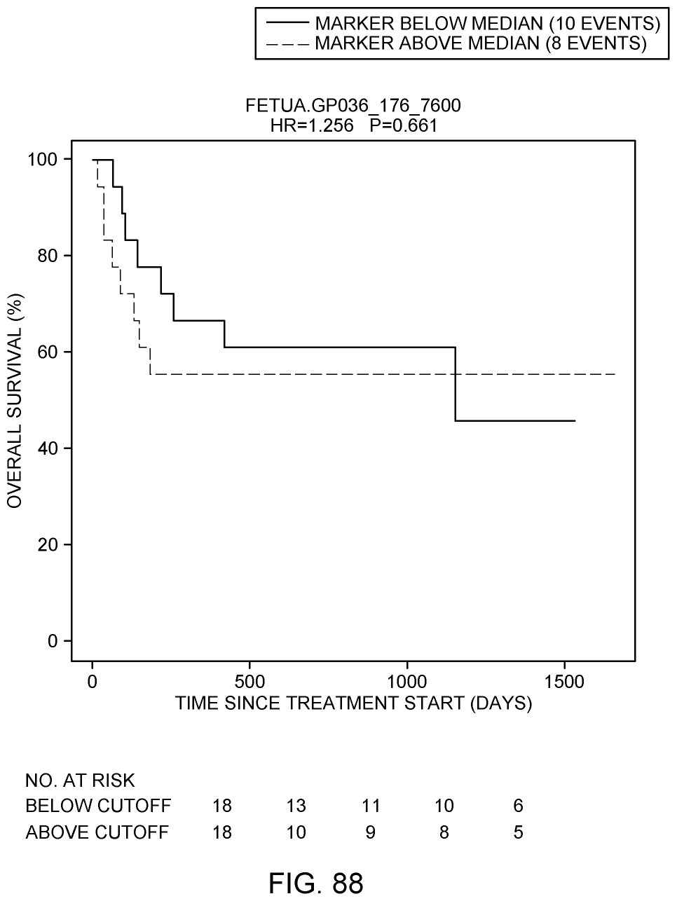

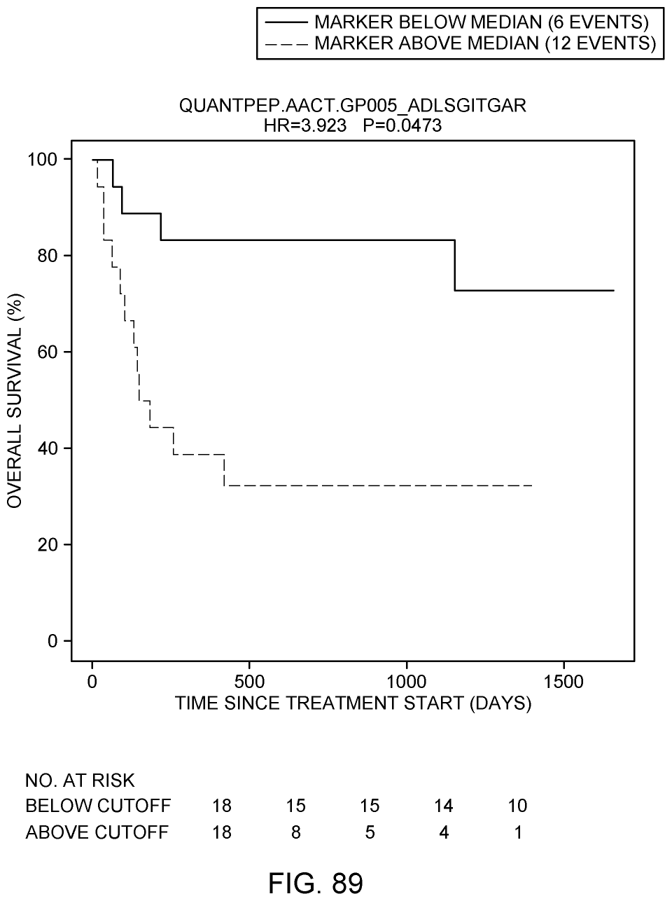

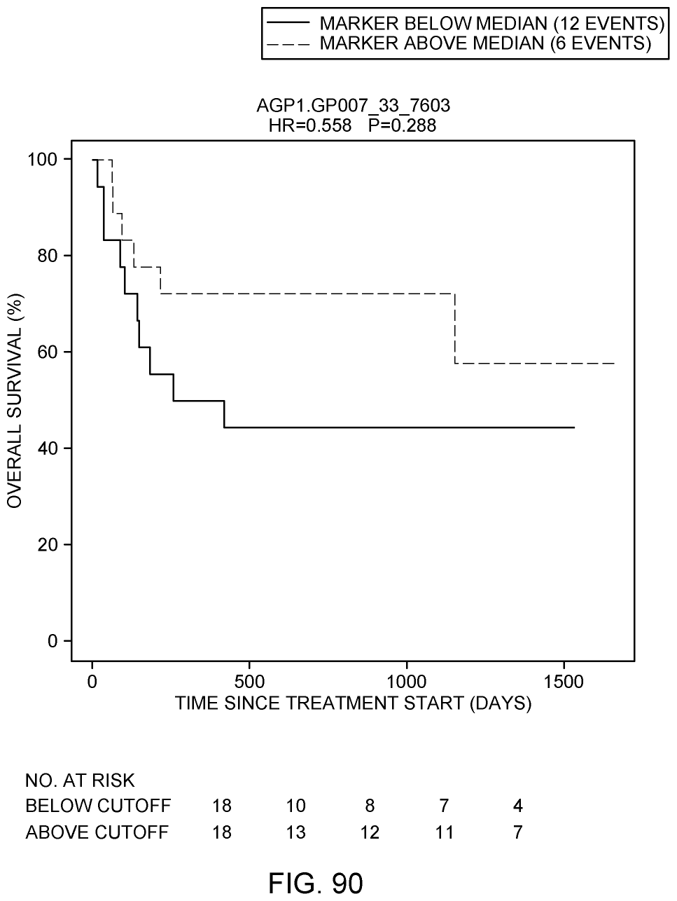

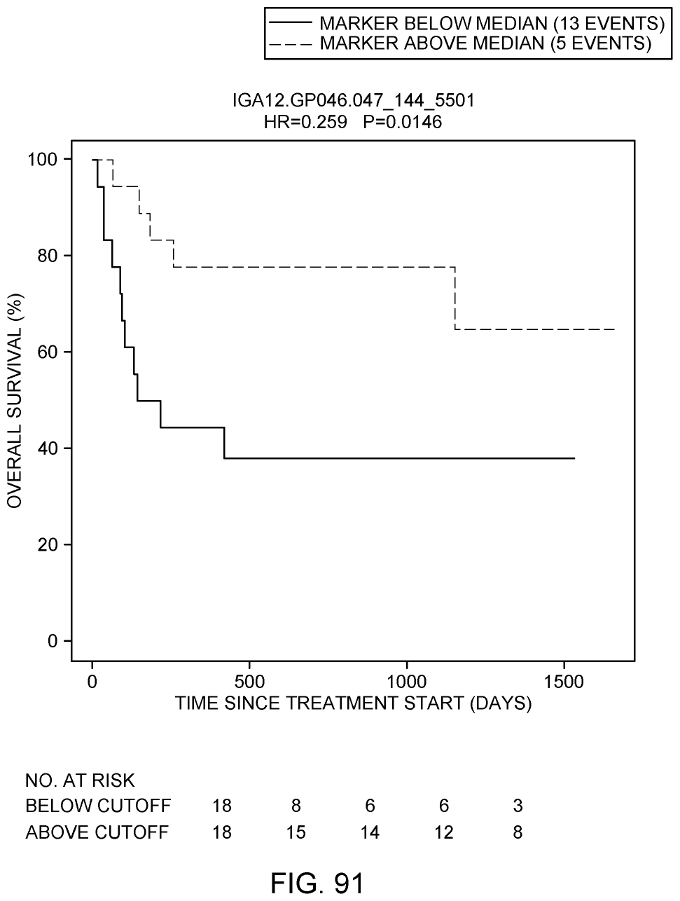

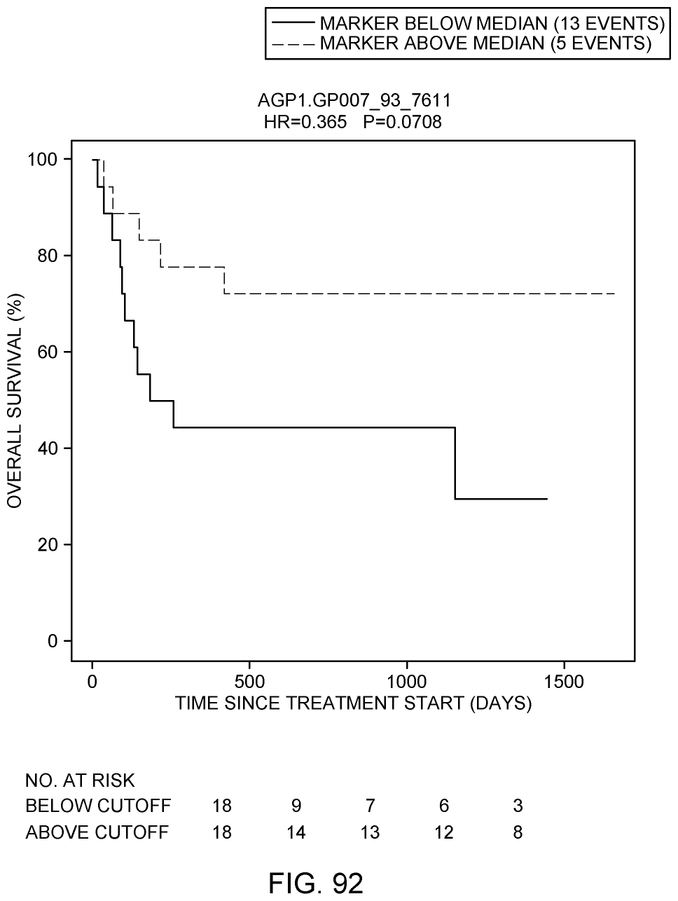

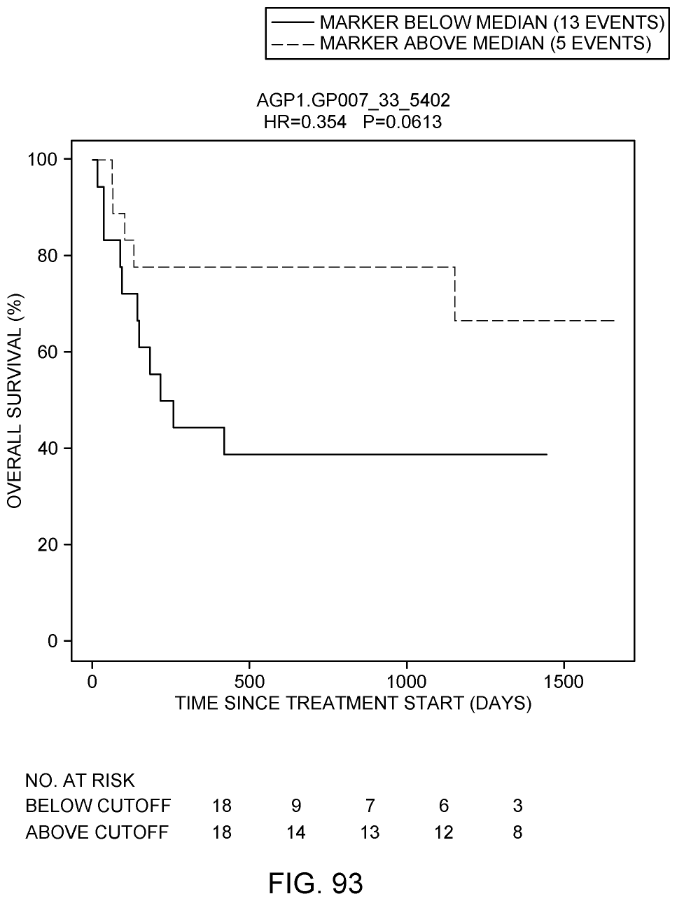

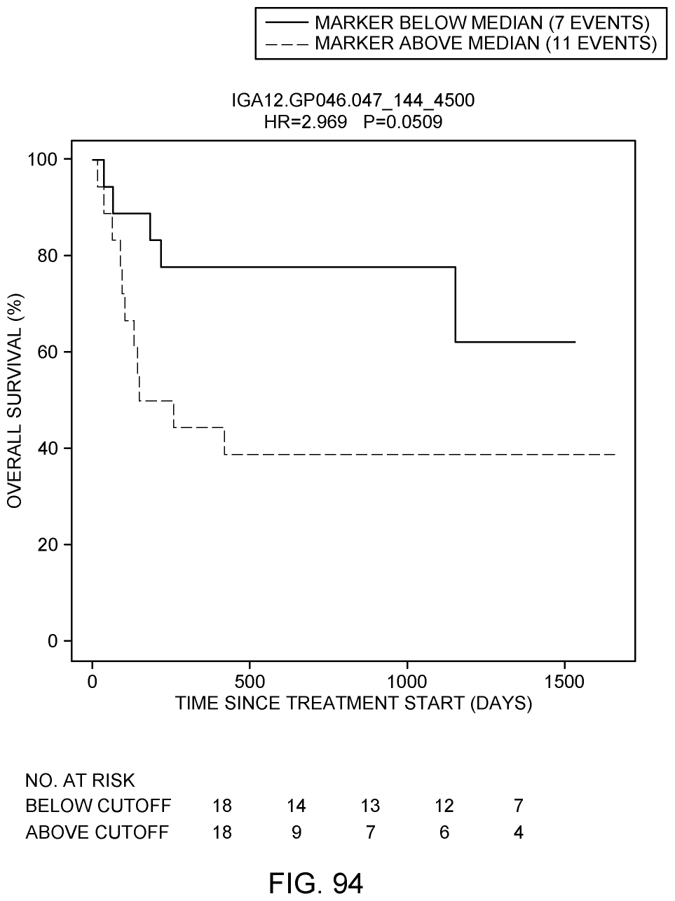

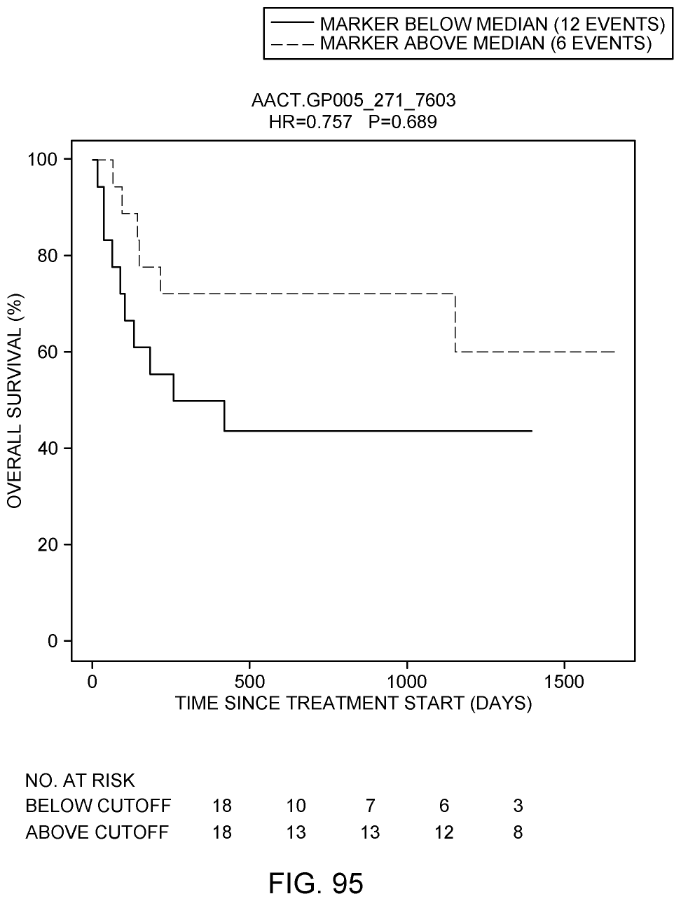

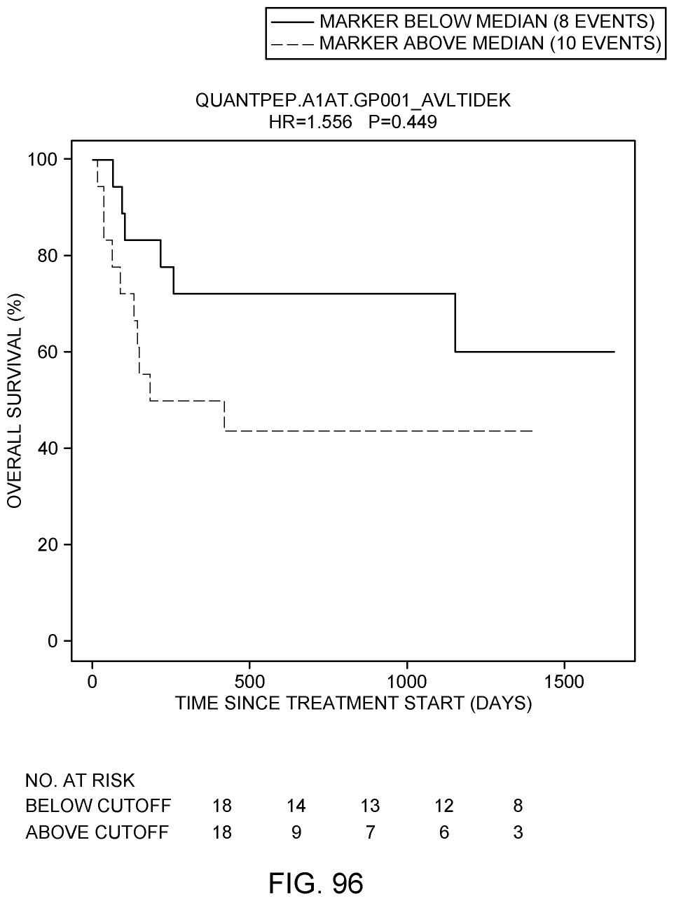

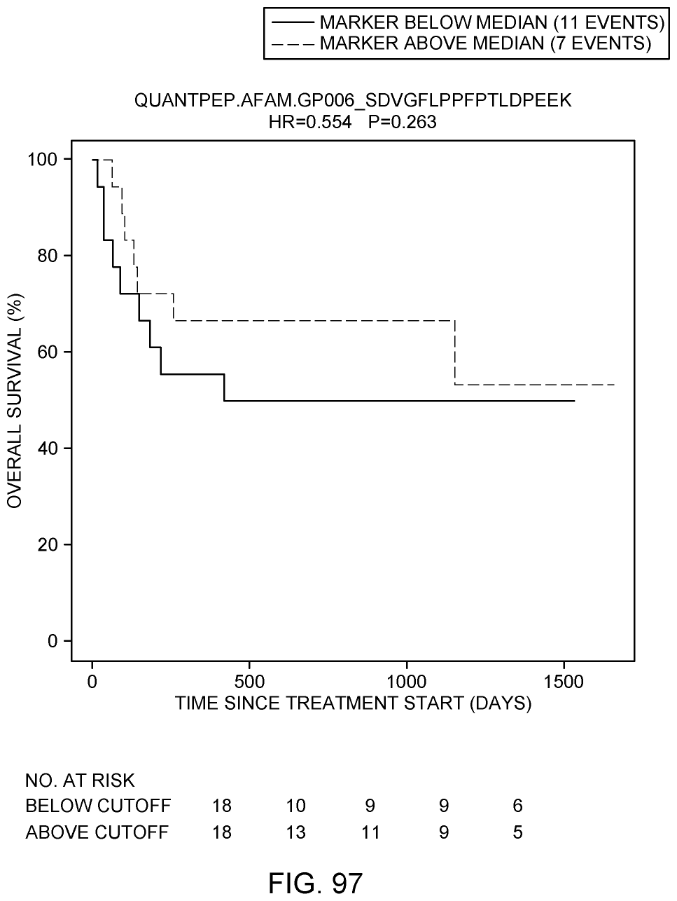

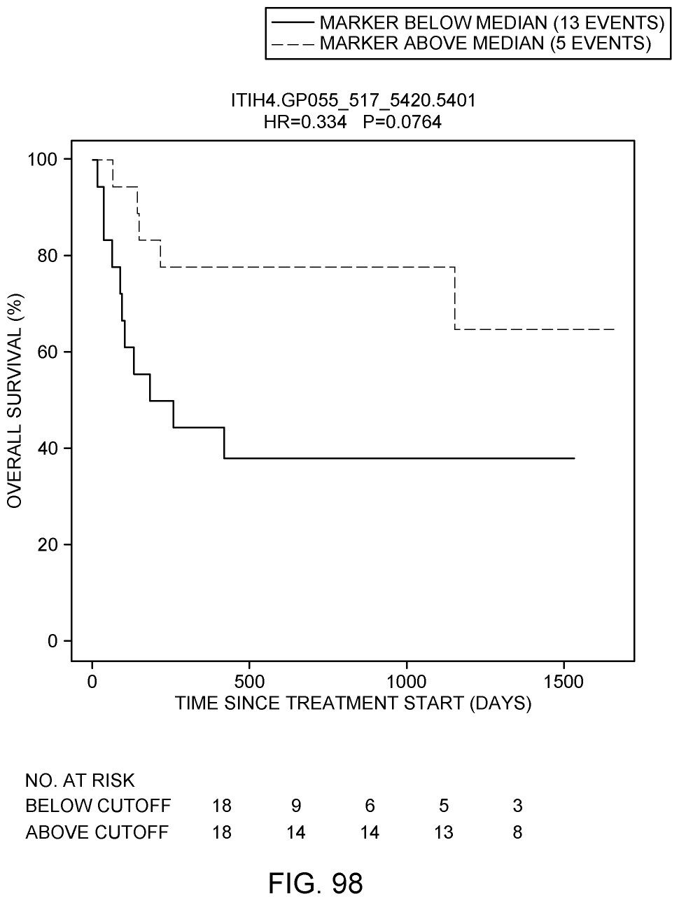

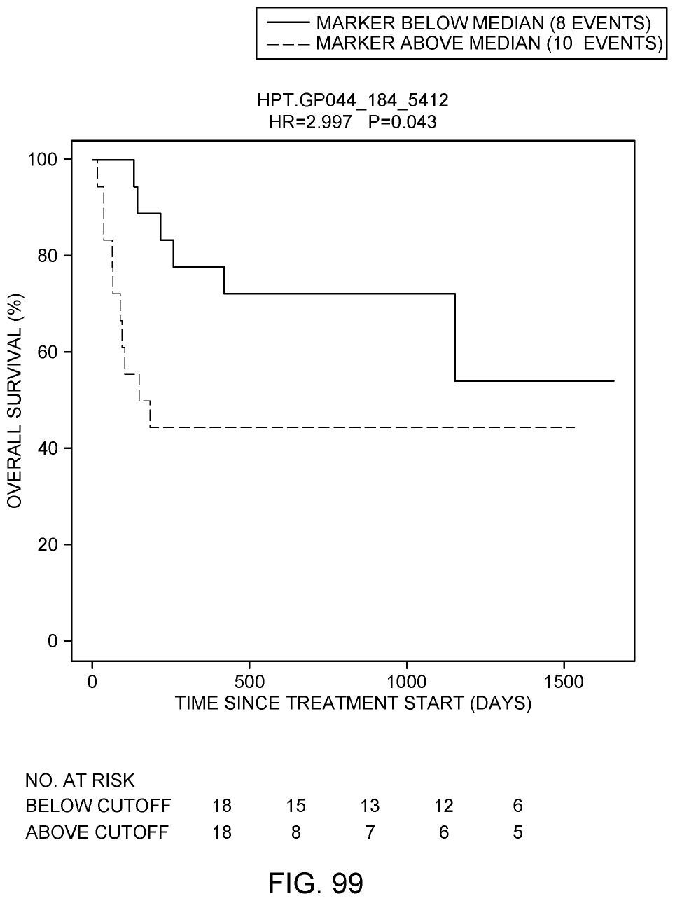

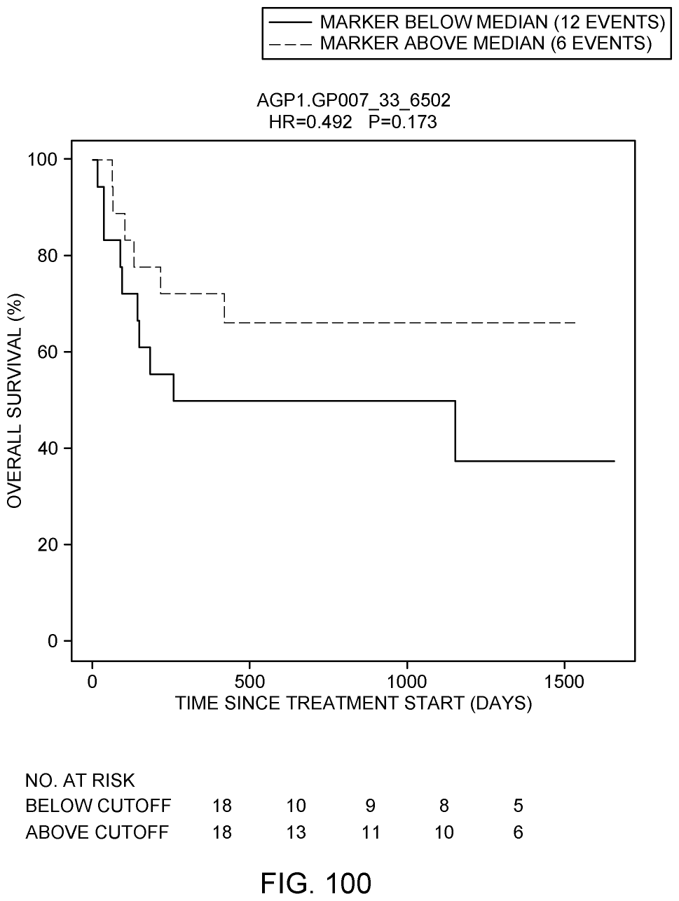

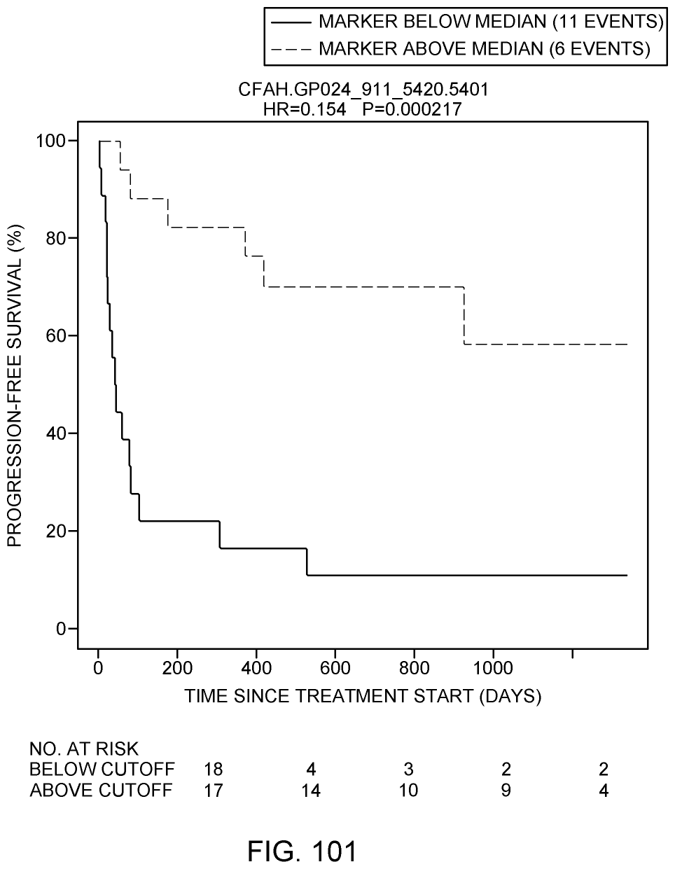

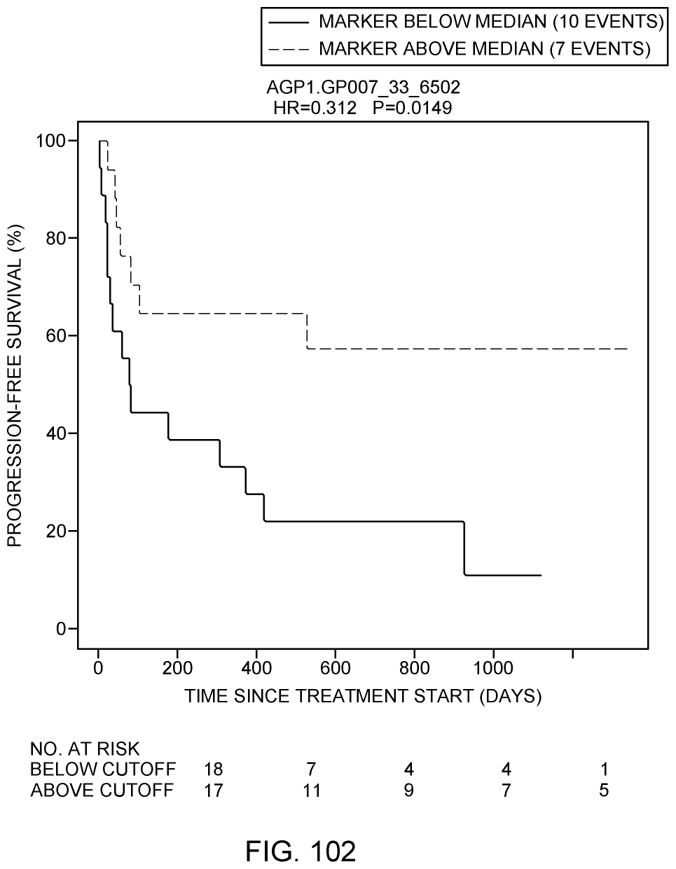

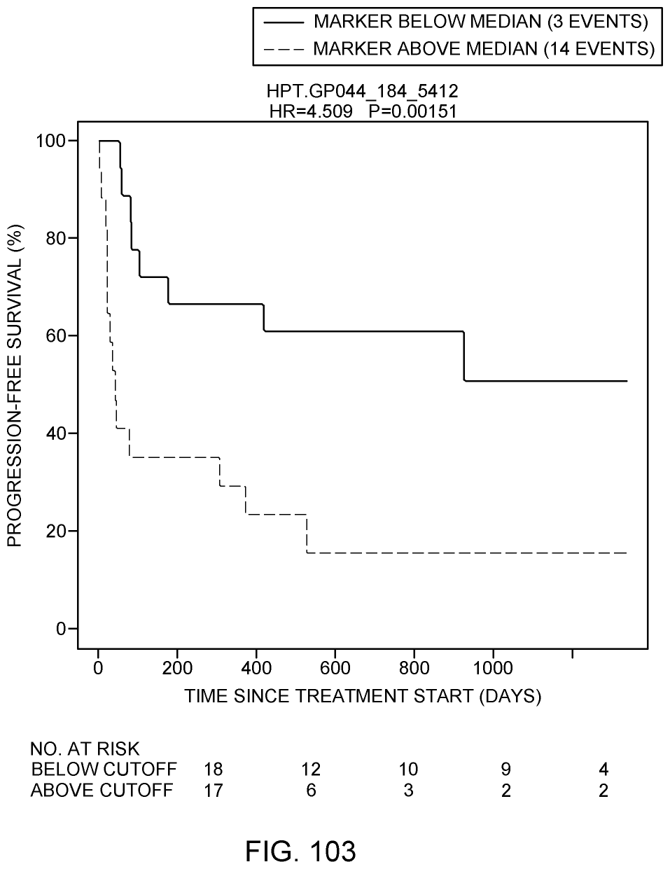

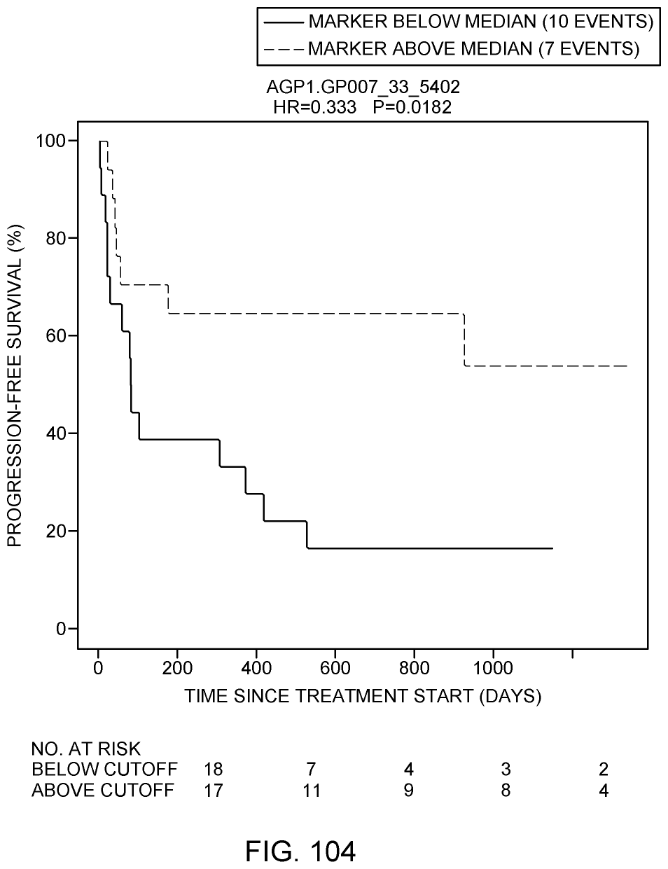

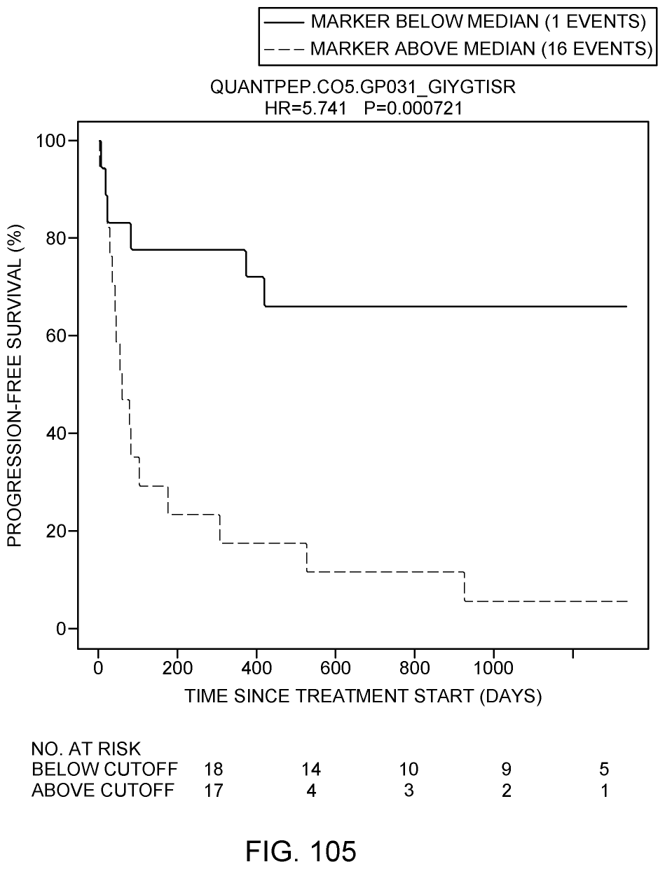

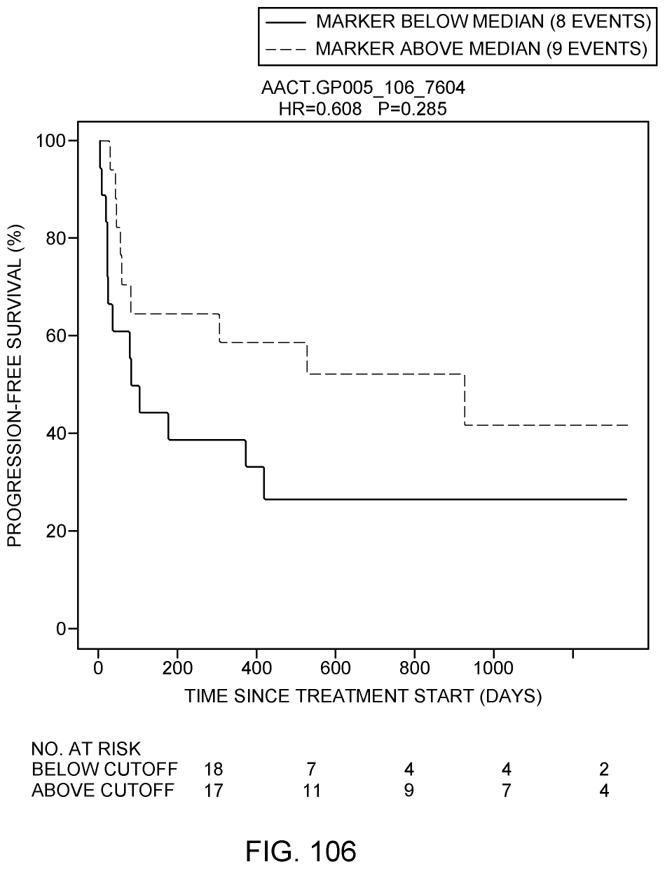

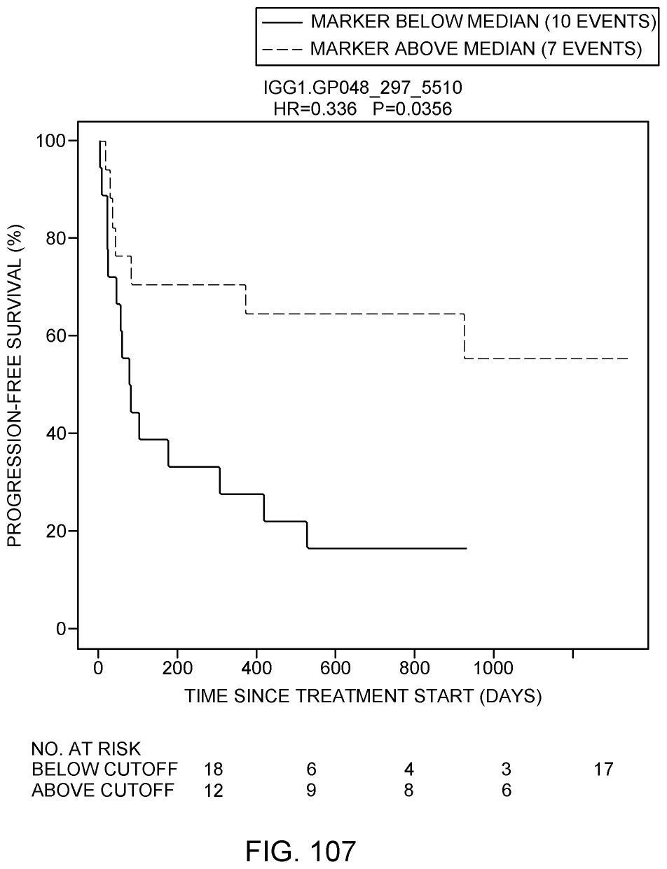

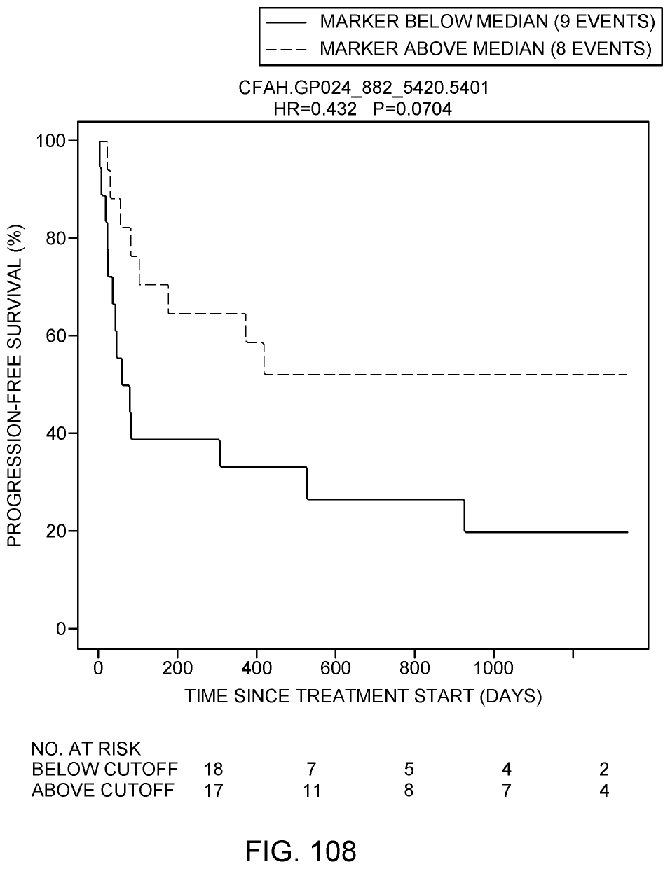

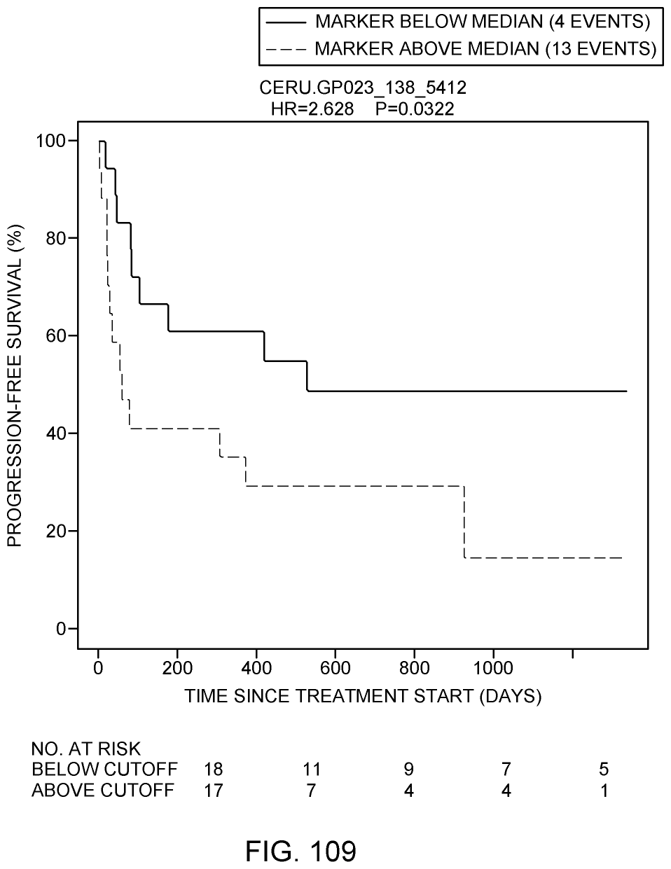

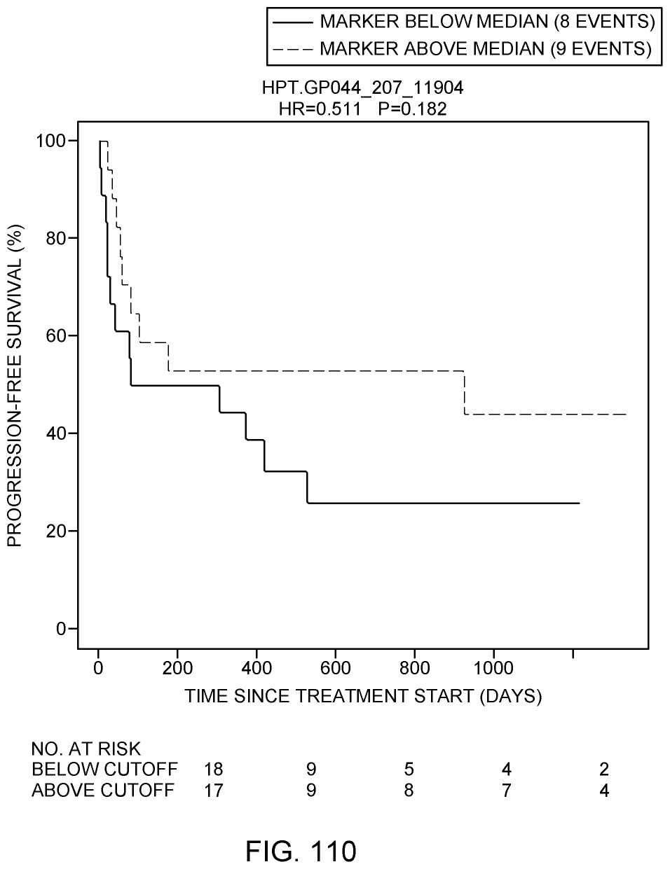

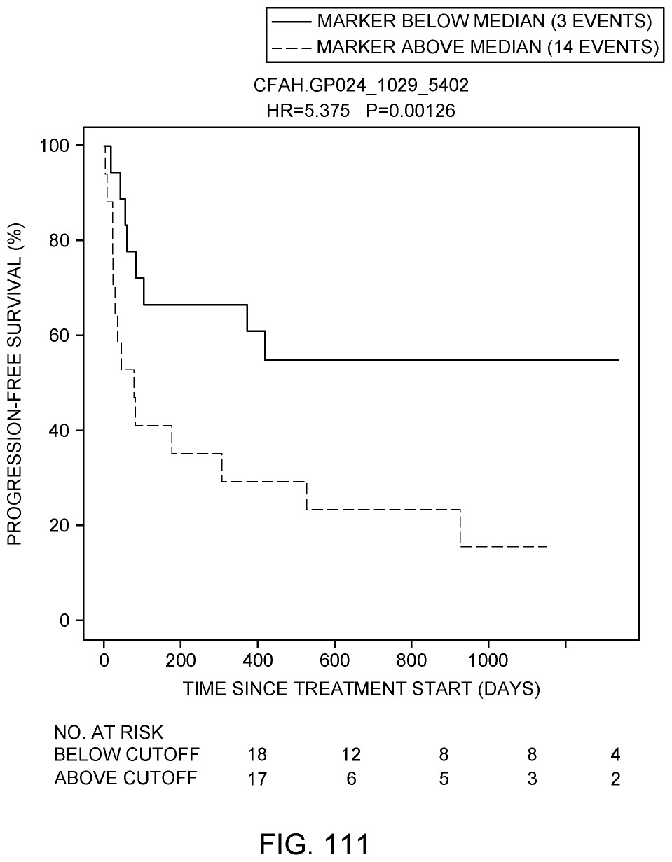

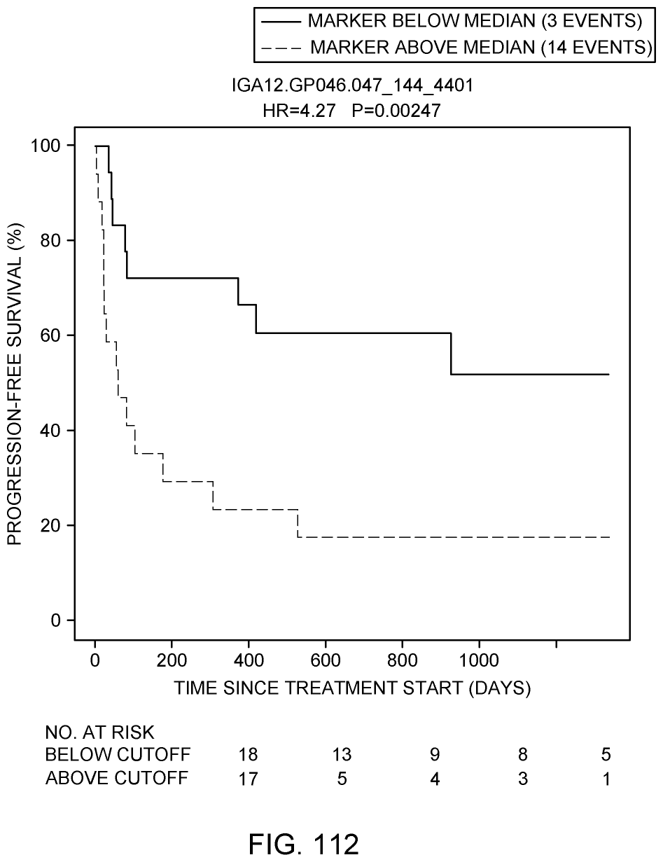

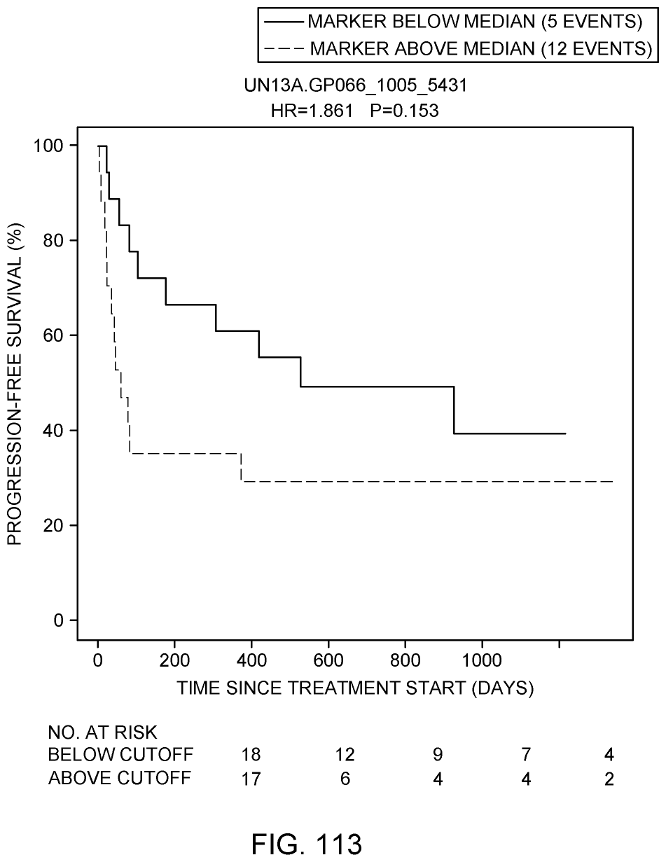

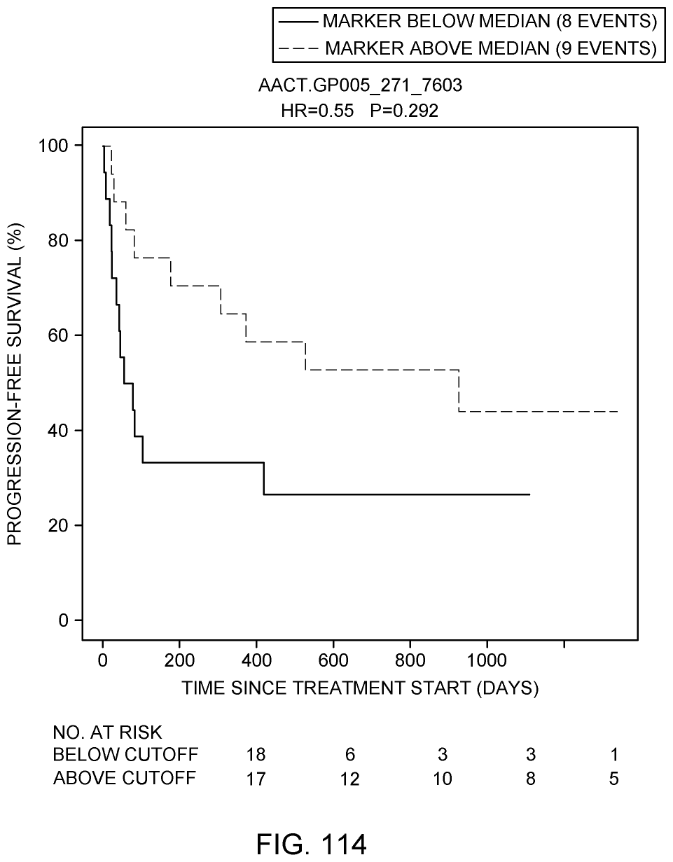

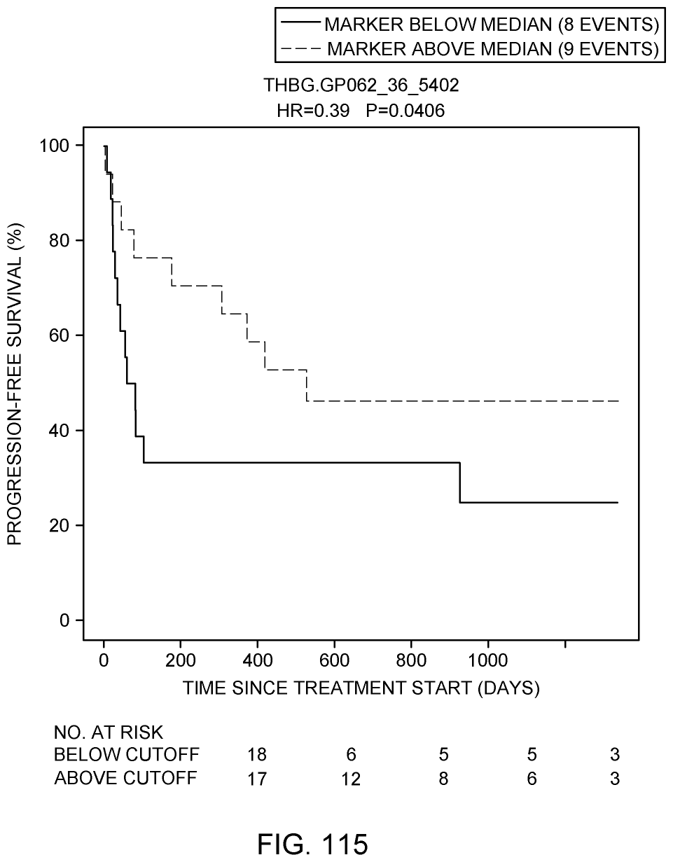

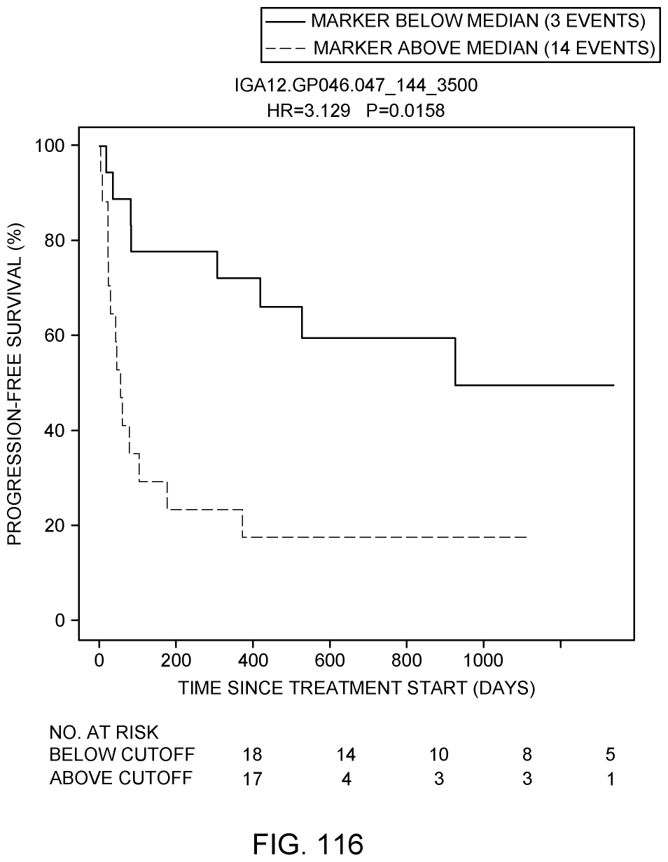

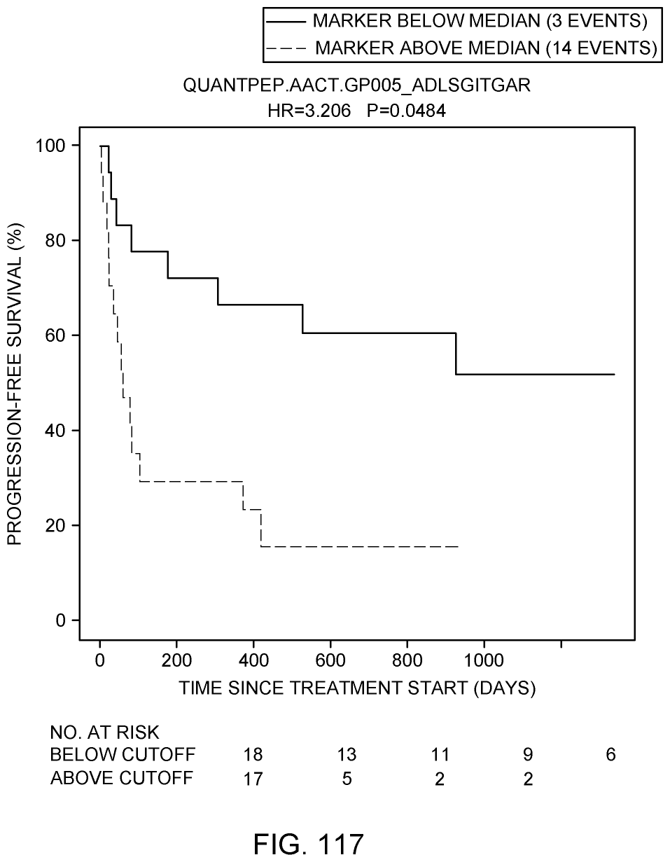

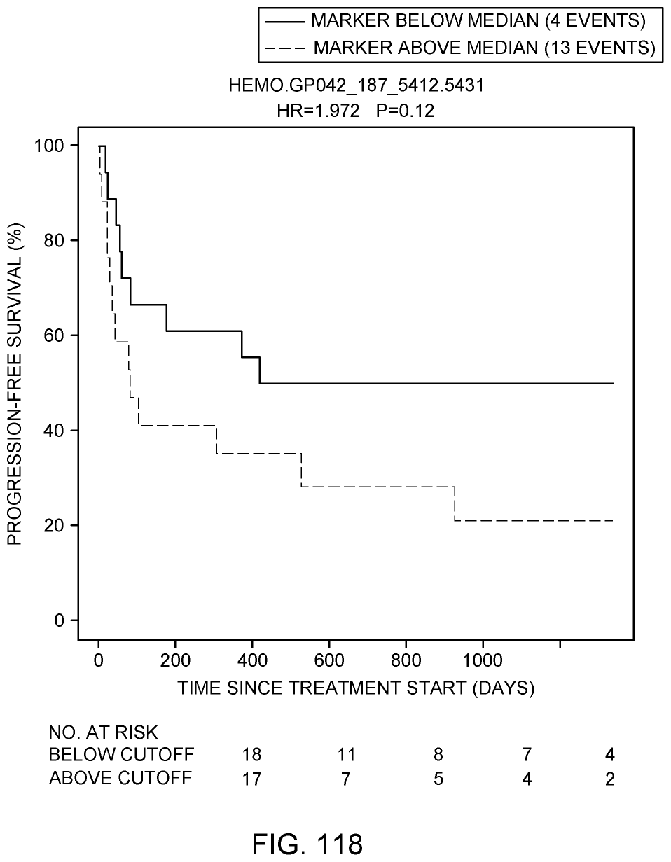

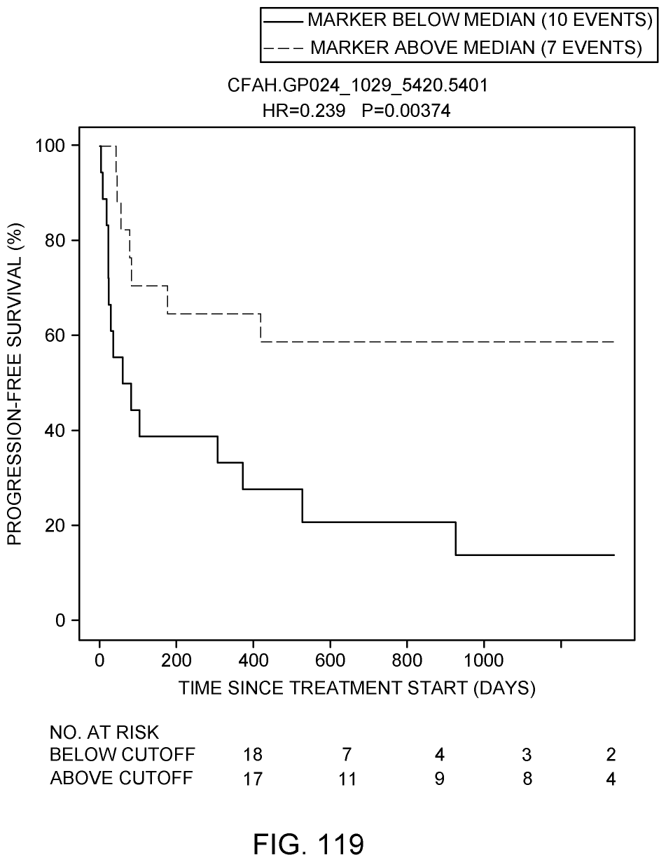

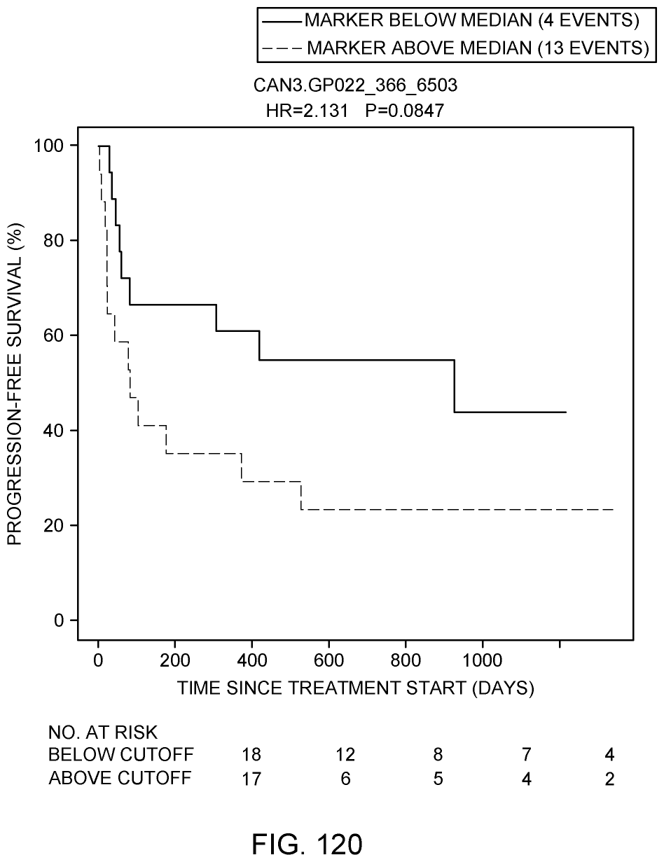

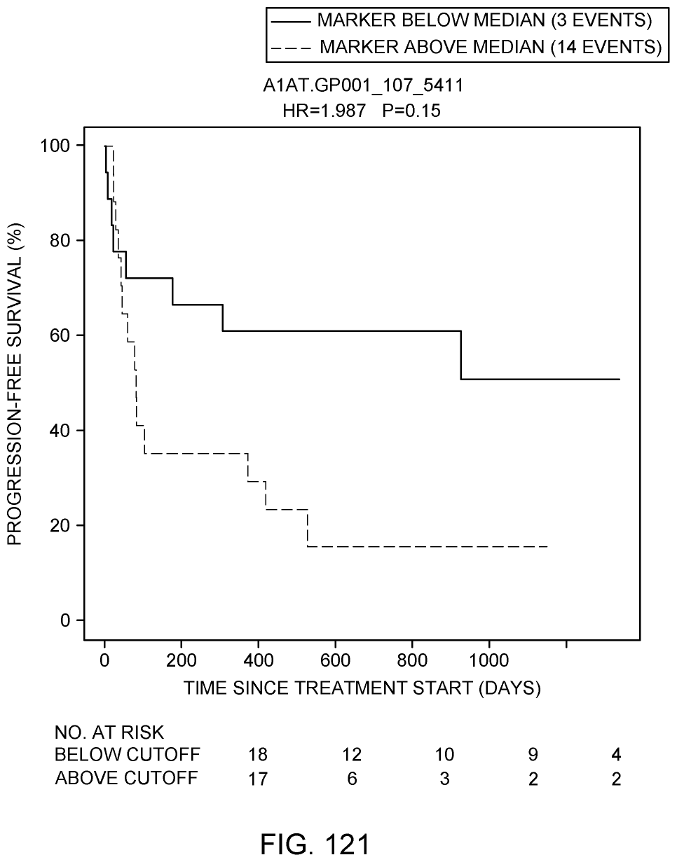

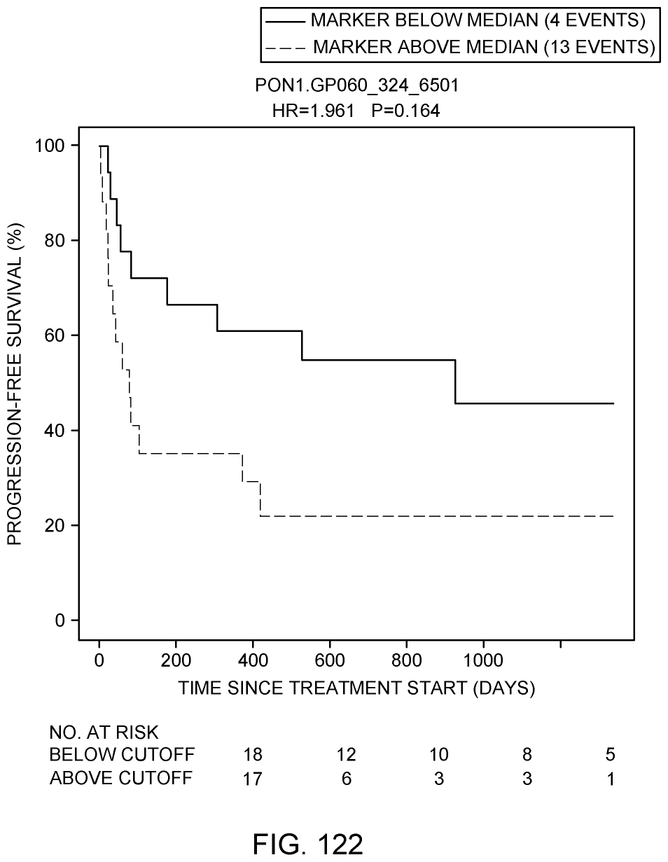

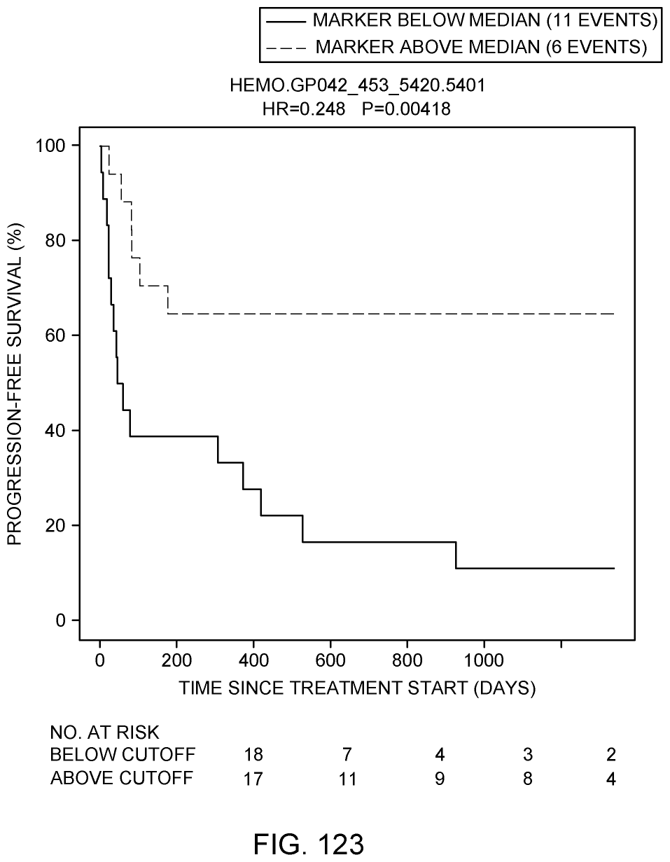

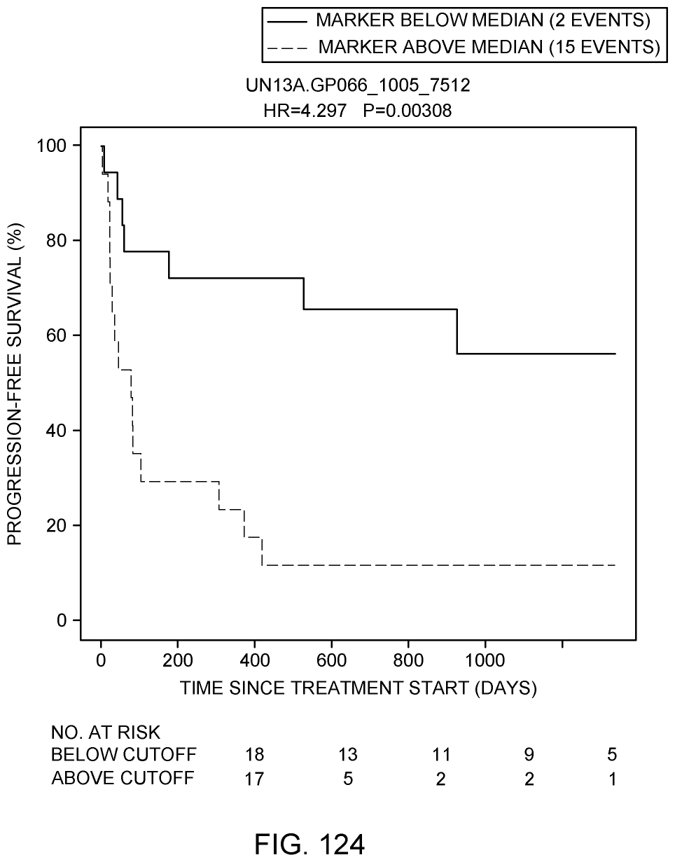

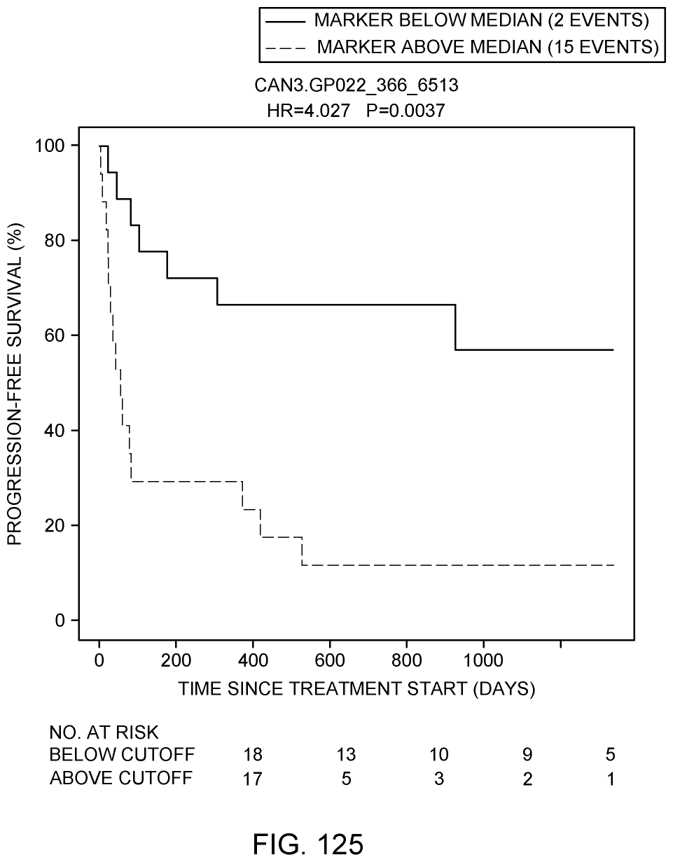

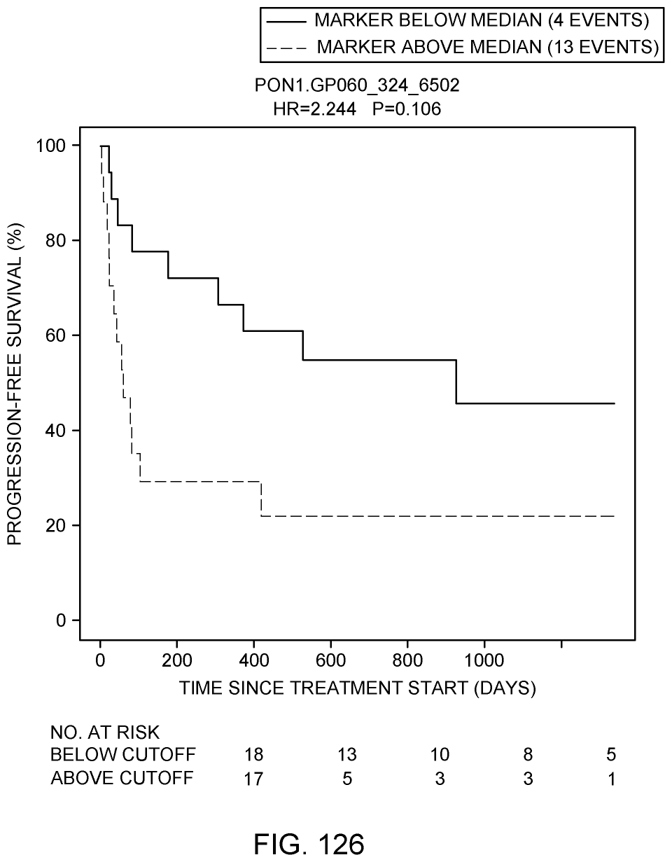

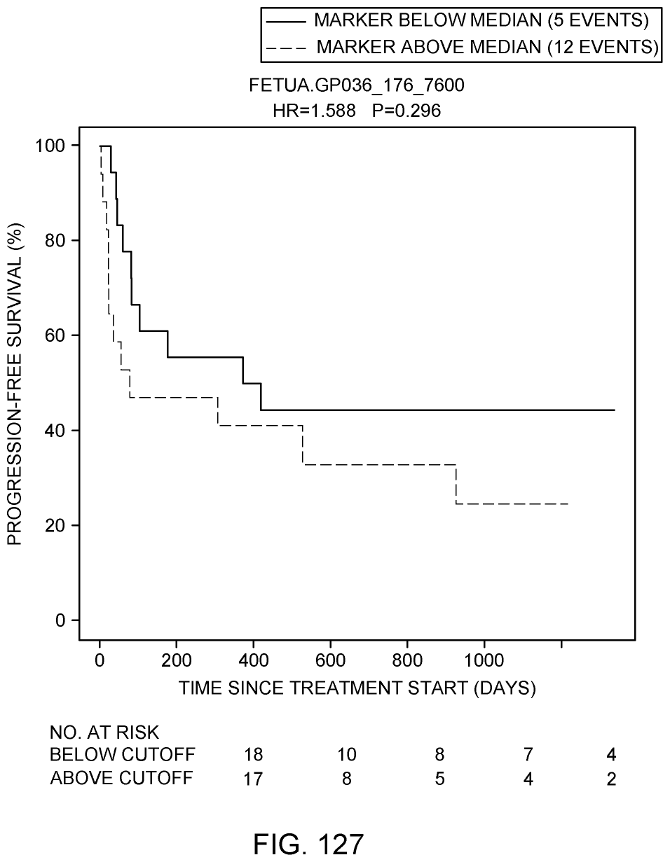

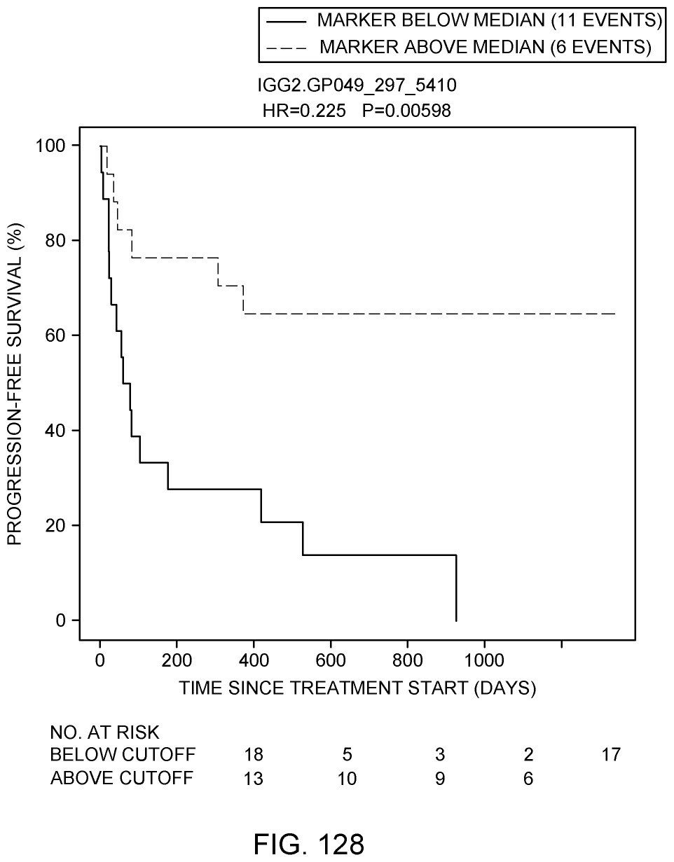

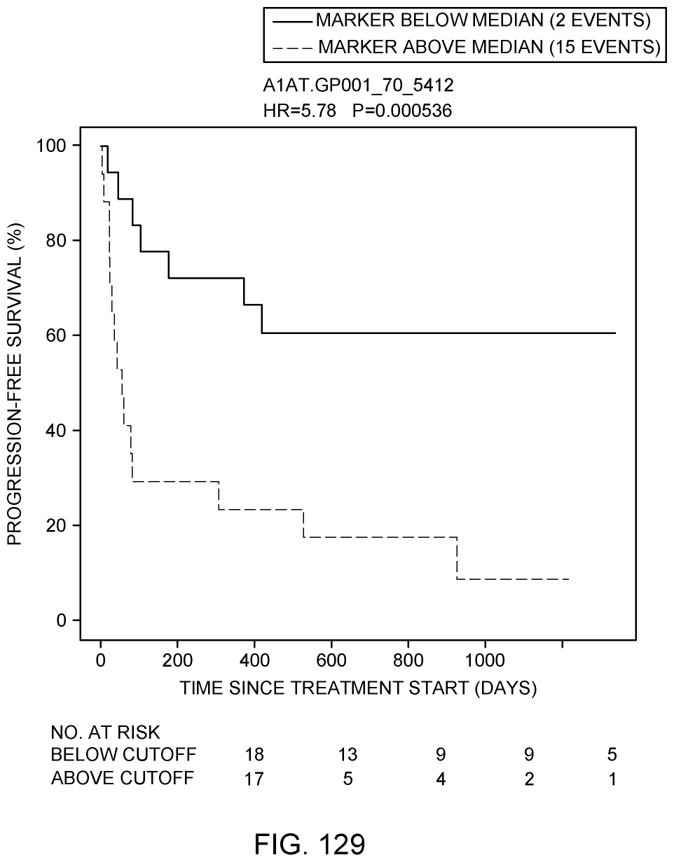

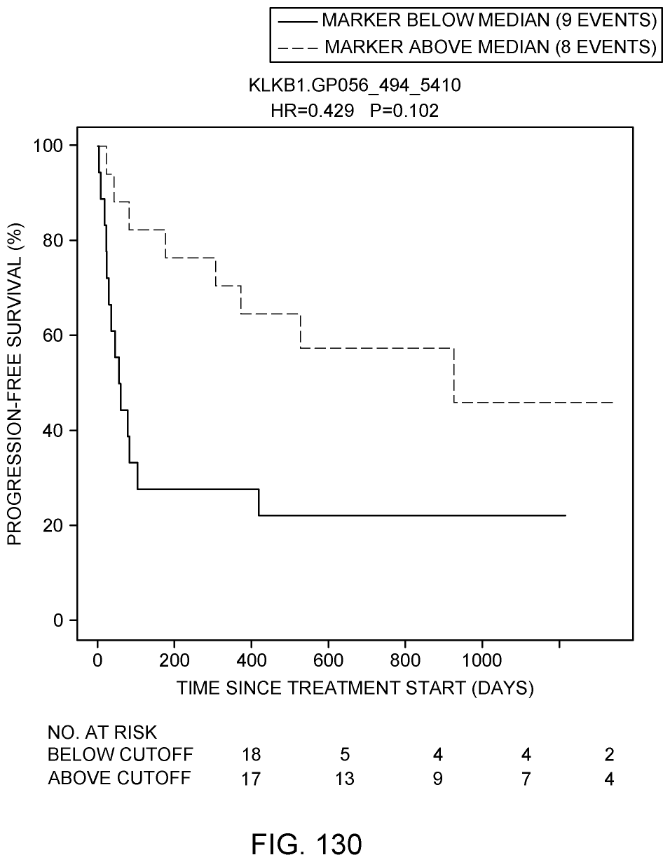

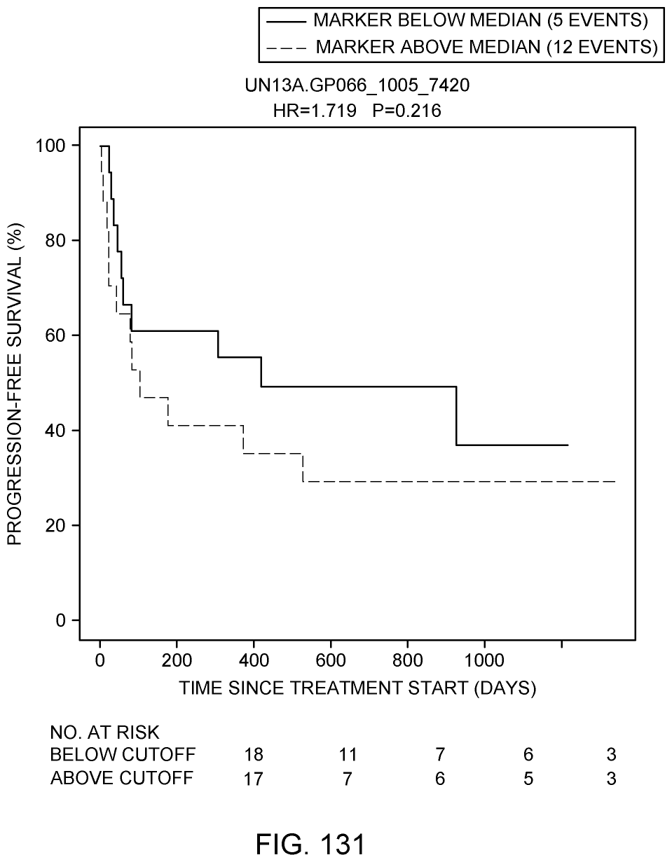

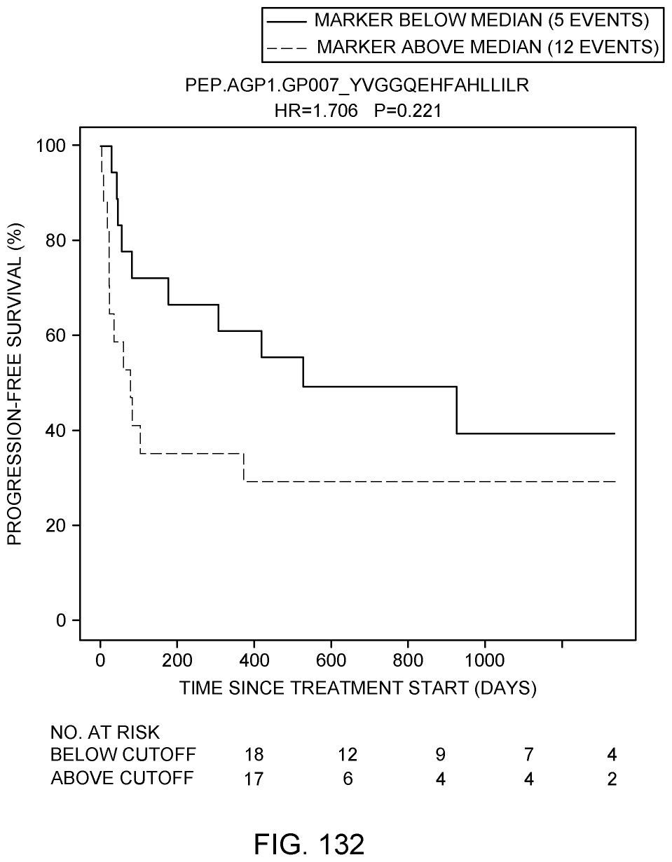

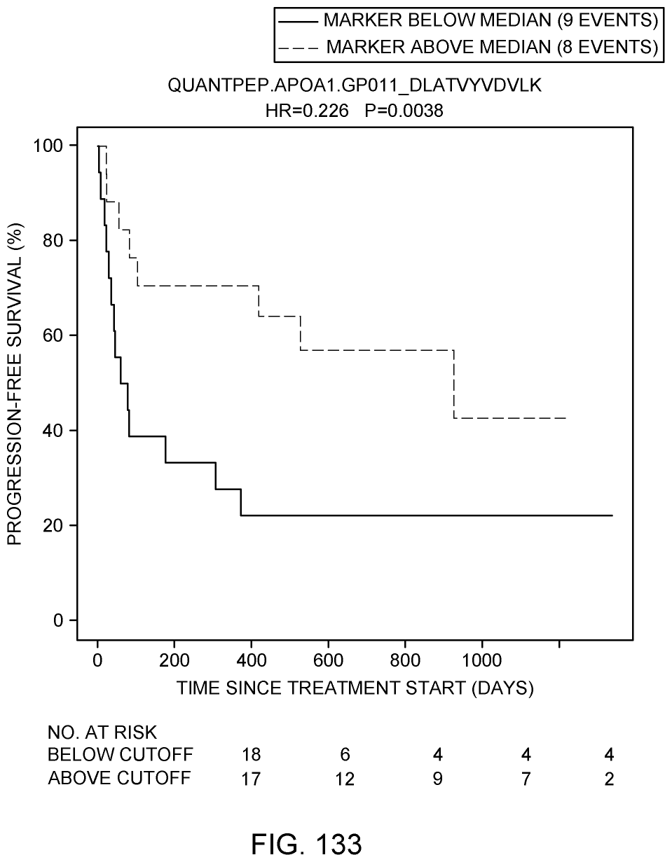

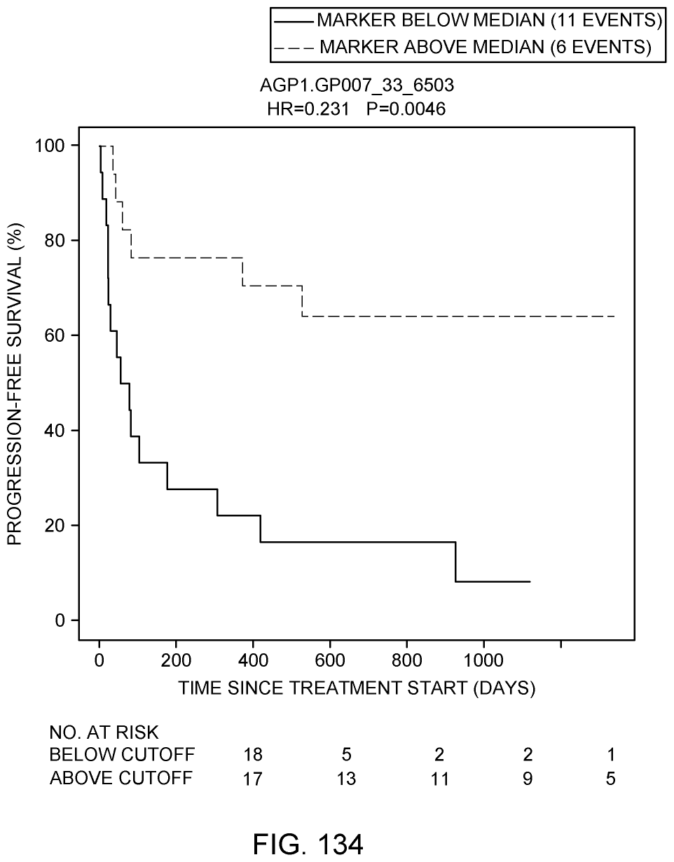

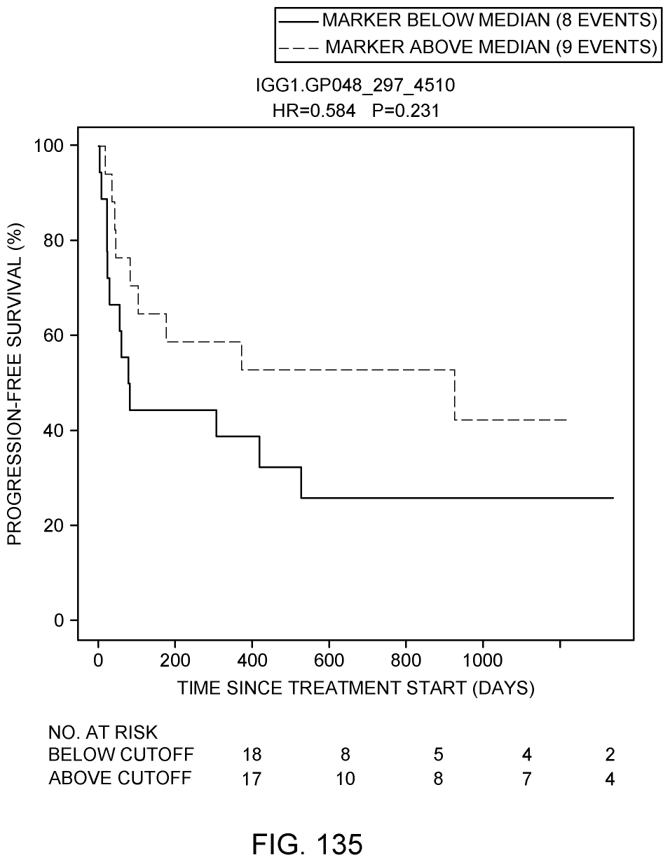

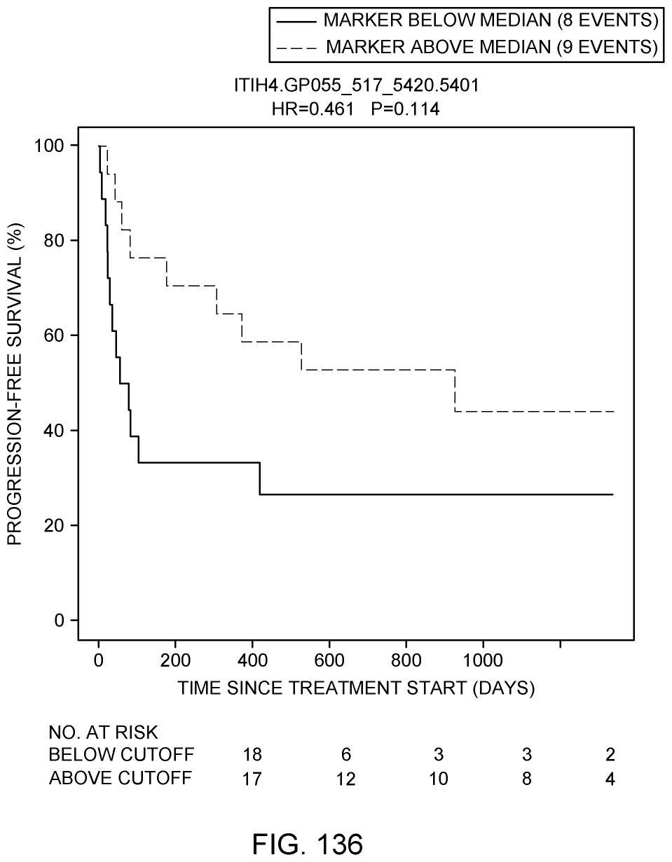

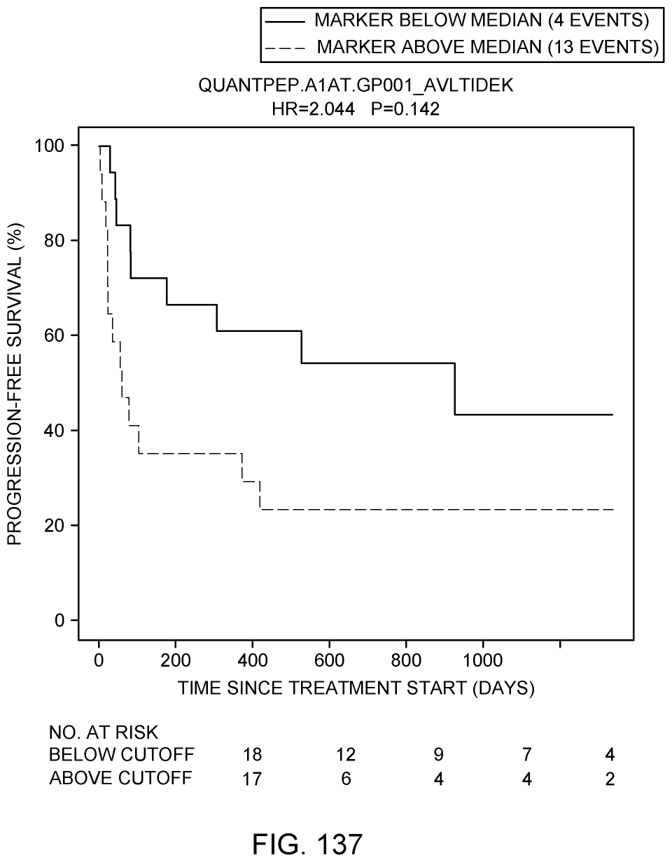

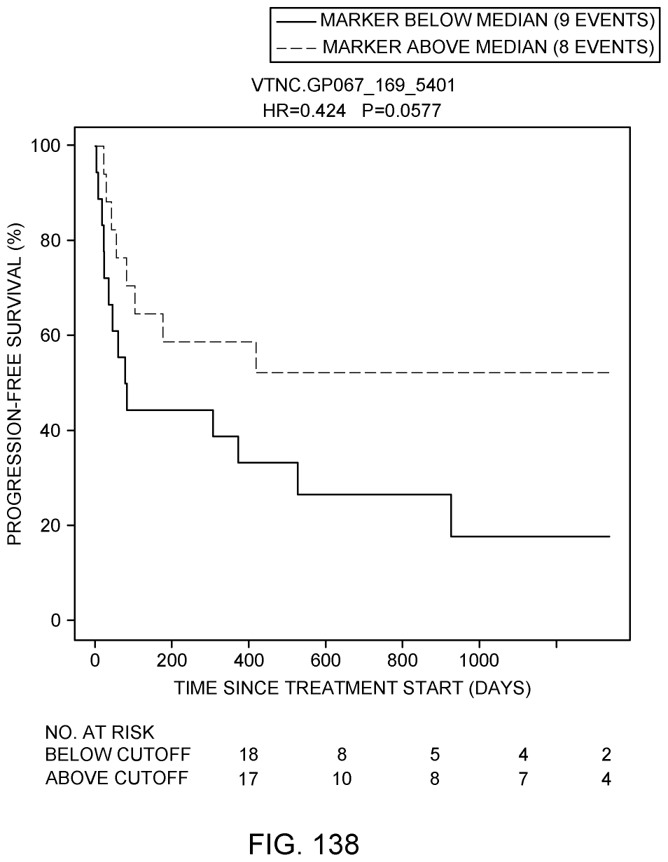

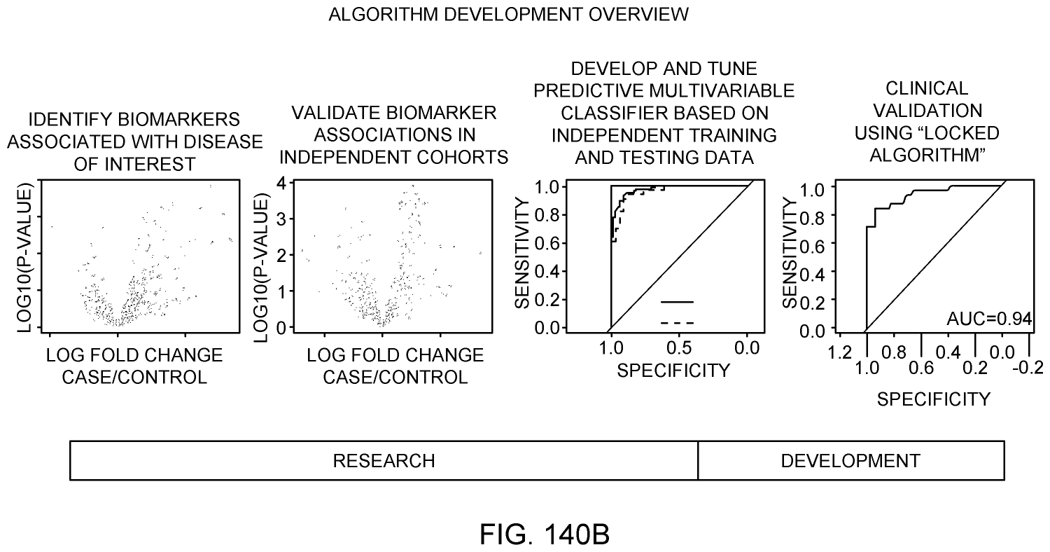

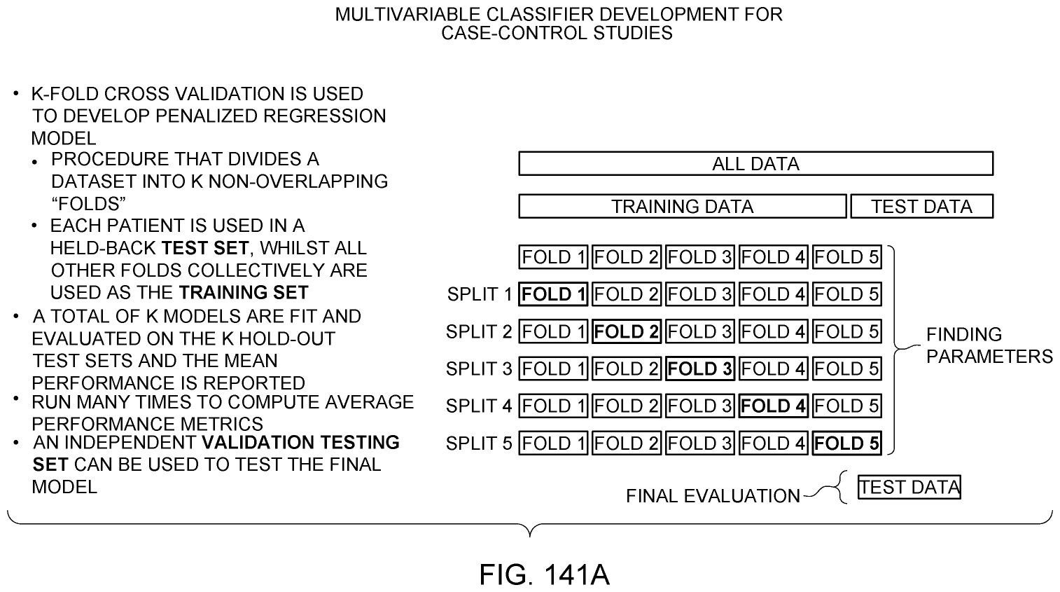

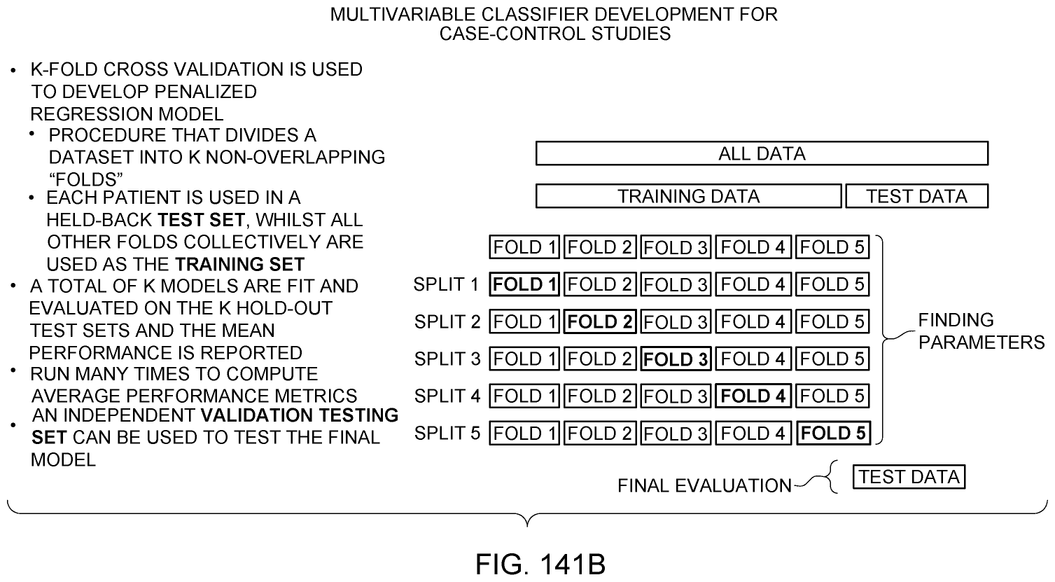

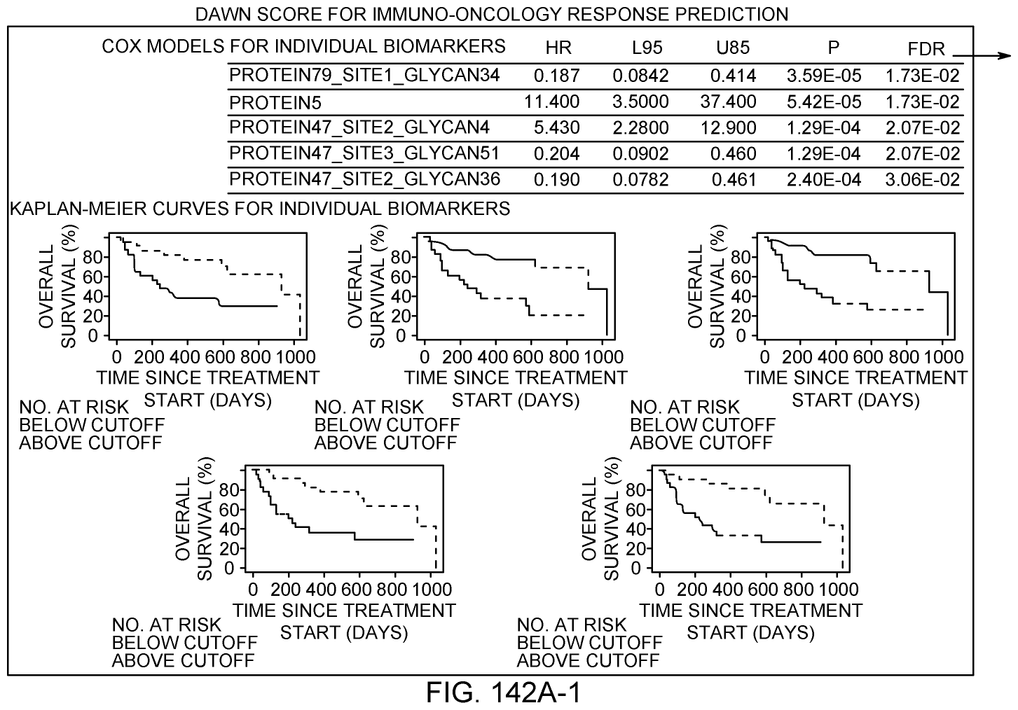

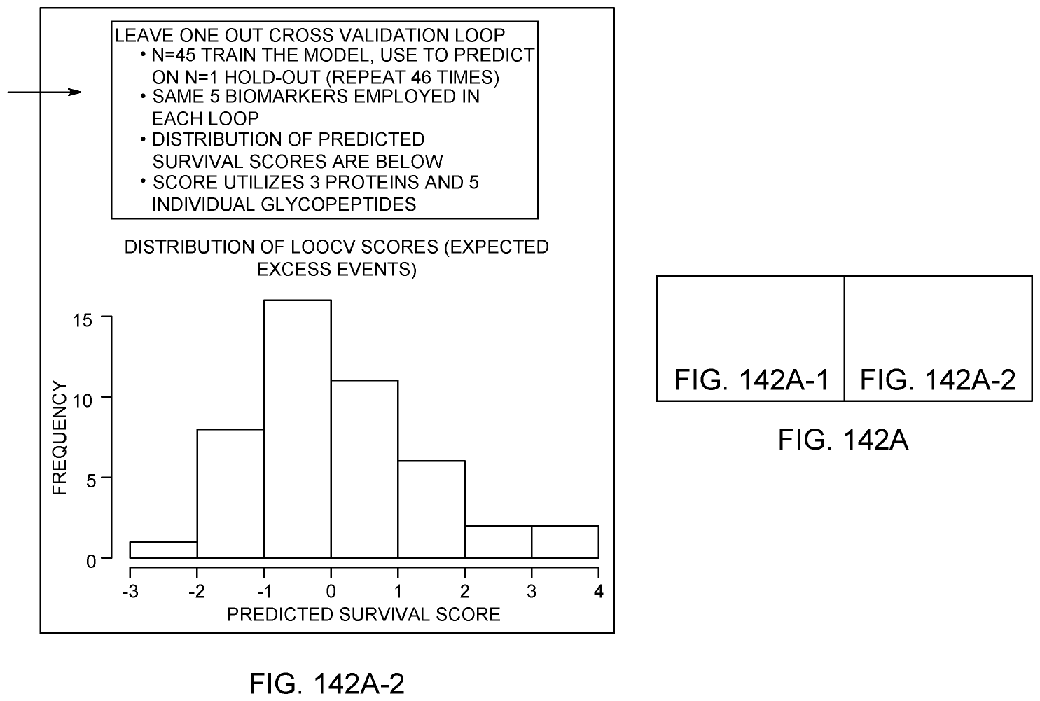

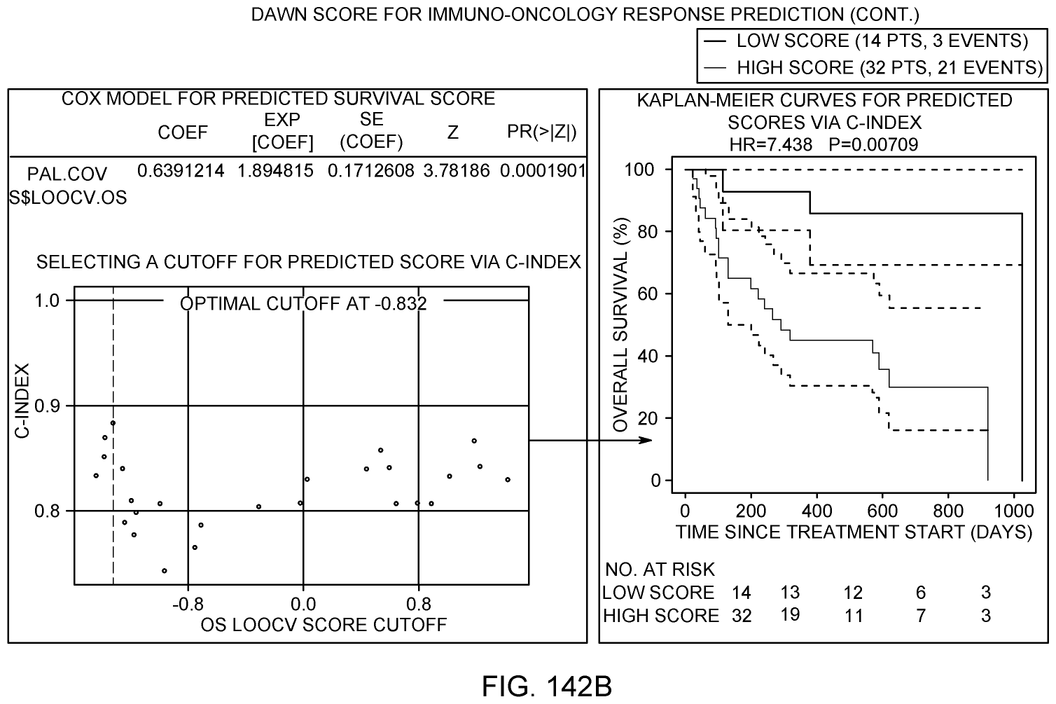

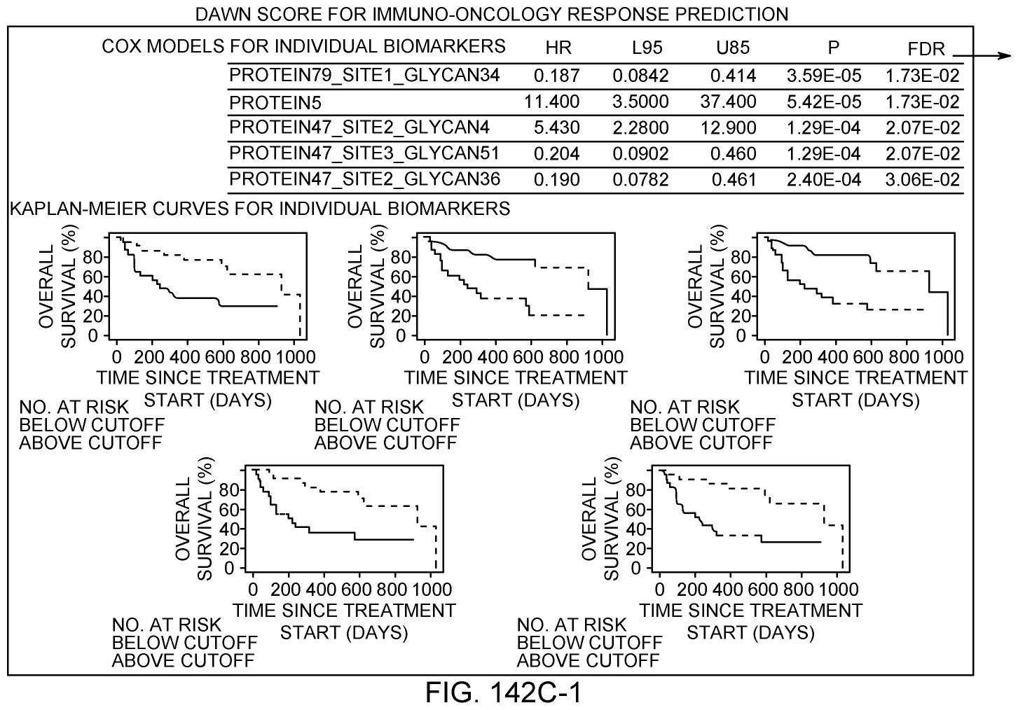

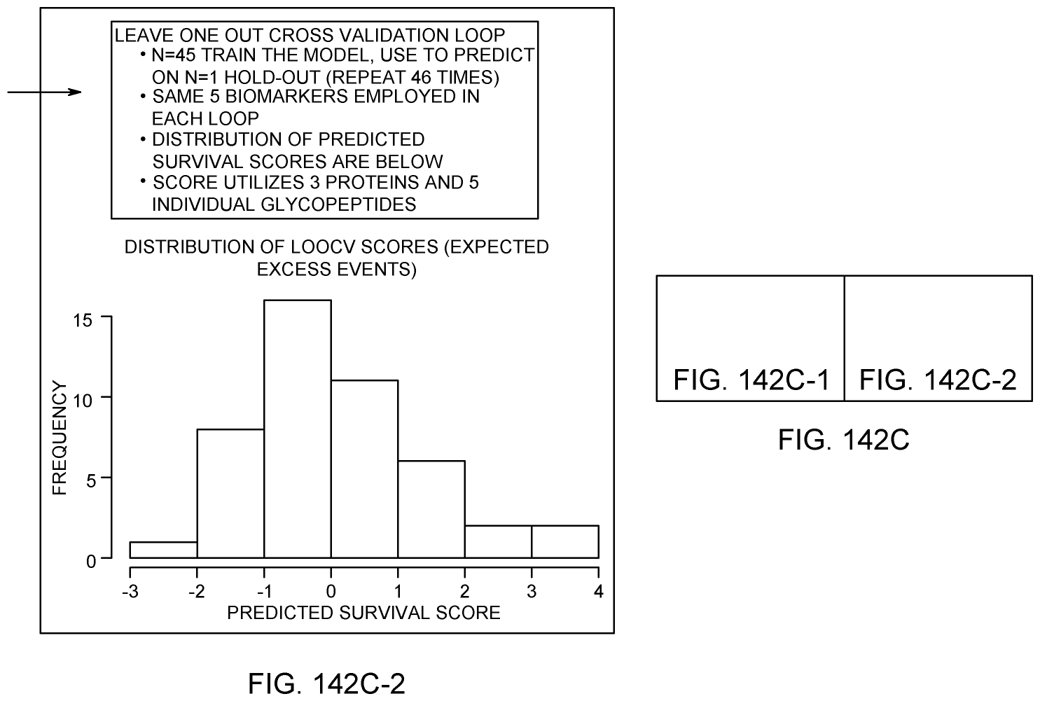

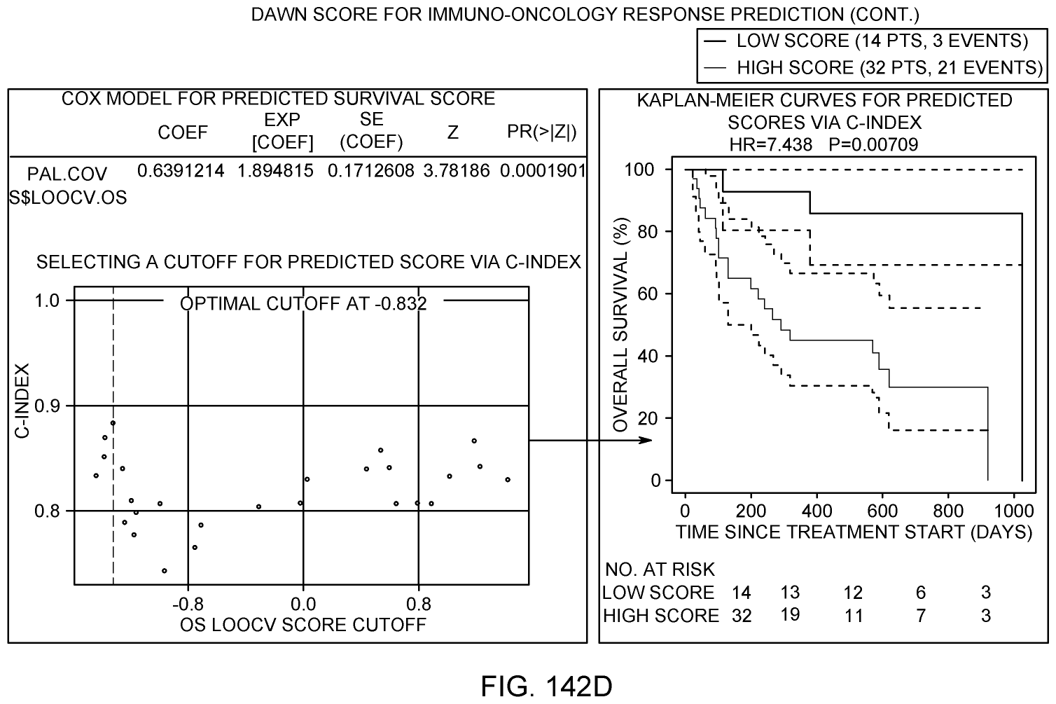

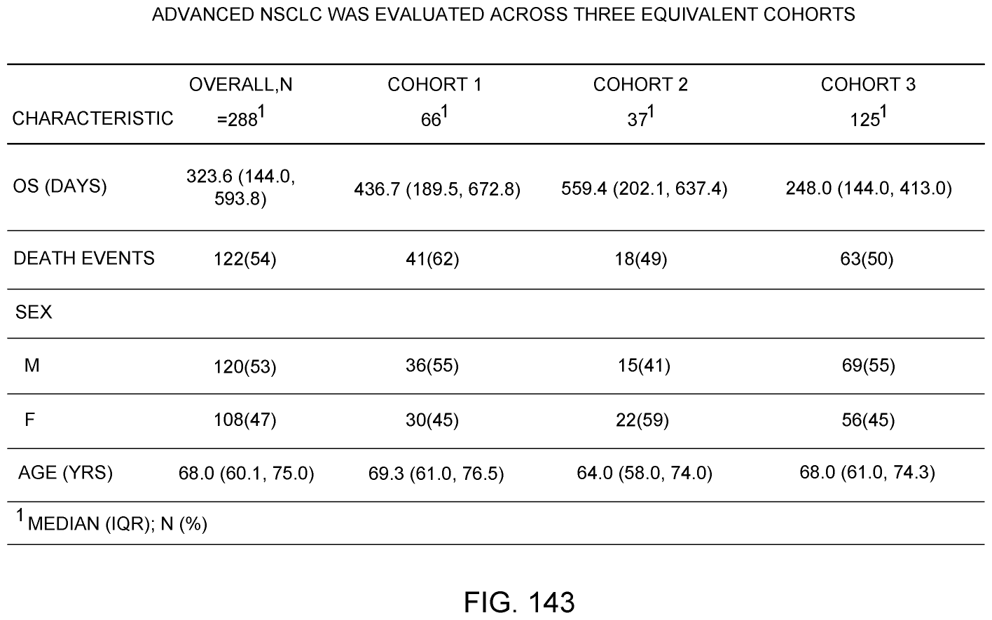

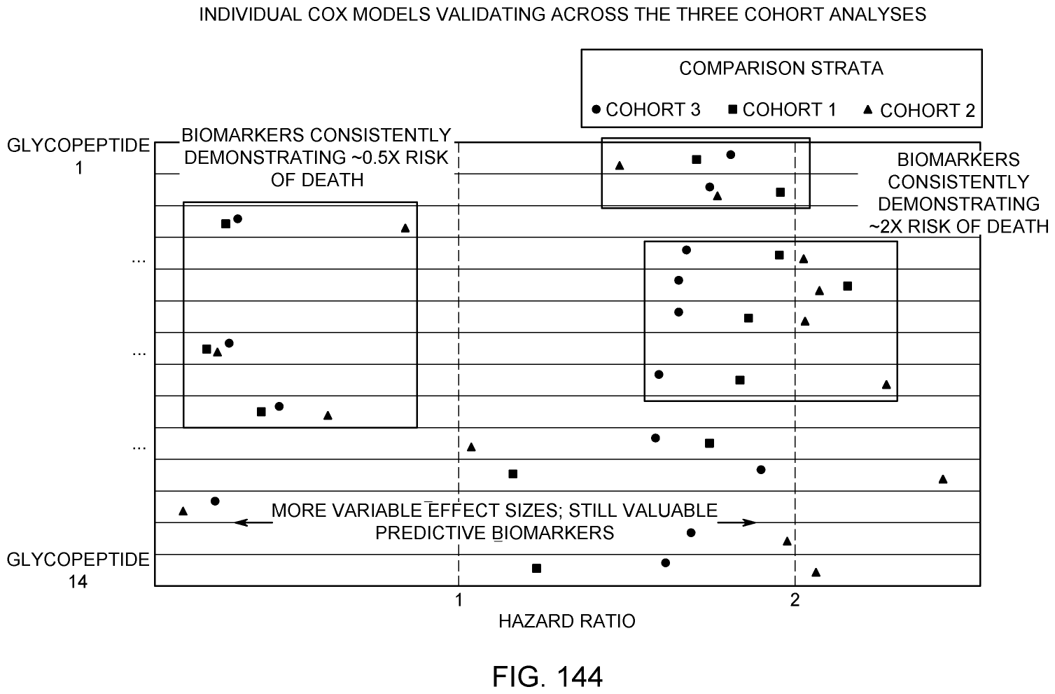

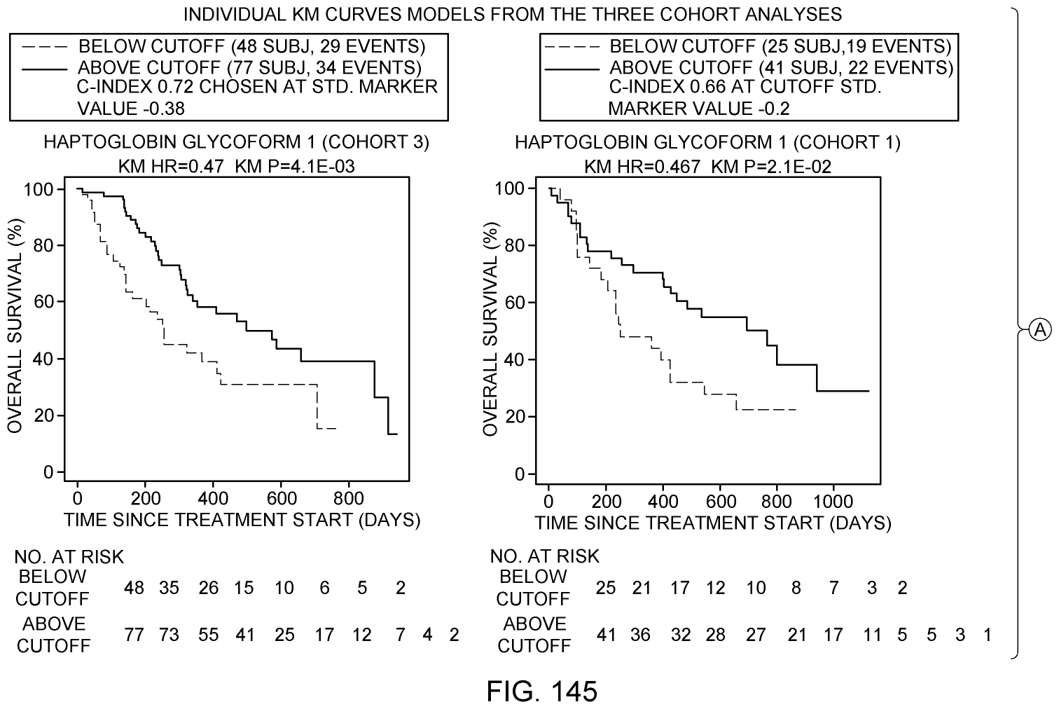

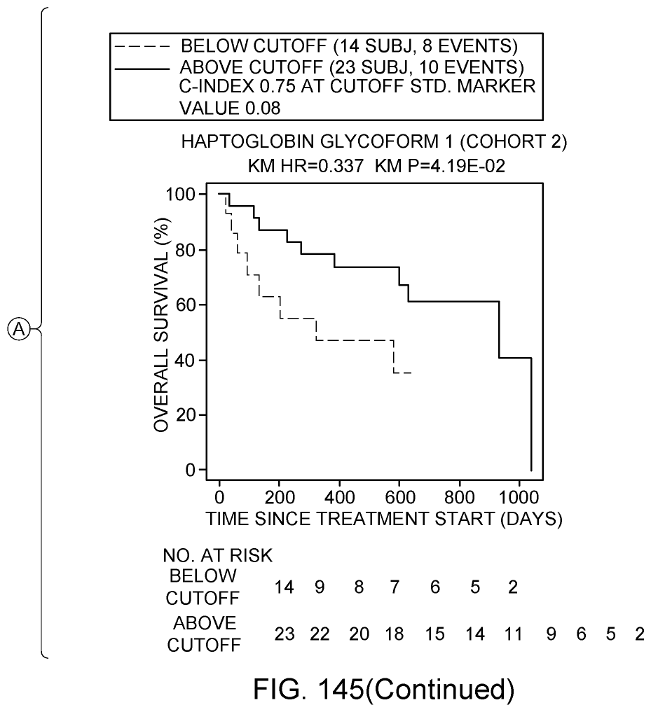

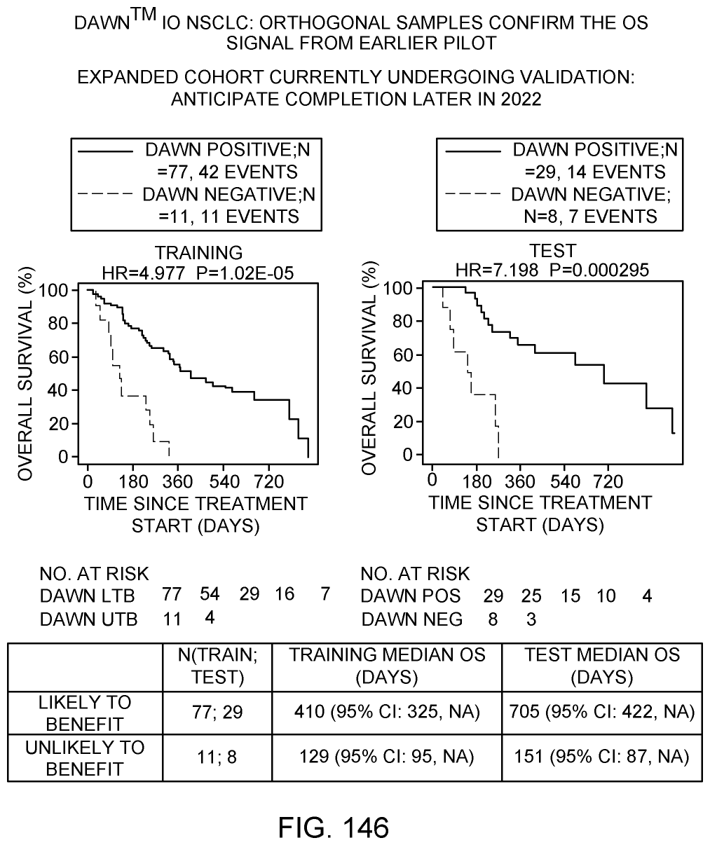

In some embodiments, the model helped to determine whether the glycopeptide marker individually predictive of OS. In some embodiments, the model helped to determine whether the glycopeptide marker individually predictive of PFS. In some embodiments, the model helped to determine whether the glycopeptide marker individually is of use in treatment selection or varied with and without treatment. In some embodiments, individual Kaplan-Meier (KM) curves are plotted for the markers relevant in each disease for each outcome, such as OS or PFS. In some embodiments, hazard ratios and p-values on the plots are representative of the plotted high/low split at median biomarker expression. Examples of individual KM curves are shown in FIGS. 75-139 for melanoma and NSCLC. FIGS. 75-100 show overall survival (OS) Kaplan-Meier curves of patients with metastatic melanoma for various glycopeptide fragments. FIGS. 101-139 show progression-free survival (PFS) Kaplan-Meier curves of patients with metastatic melanoma for various glycopeptide fragments. Examples of such multivariate KM curves generated from the individual KM curves are seen in FIGS. 73A, 73B, 74A, and 74B. FIGS. 140A and 140B illustrate an algorithm development pipeline for identifying non-small-cell lung cancer (NSCLC), in accordance with the presently disclosed embodiments. FIGS. 141A and 141B illustrate a multivariate classifier development for case-control studies for identifying non-small-cell lung cancer (NSCLC), in accordance with the presently disclosed embodiments. FIGS. 142A-142D illustrate scoring prediction curves for identifying non-small-cell lung cancer (NSCLC), in accordance with the presently disclosed embodiments.

In some embodiments, patients are treated with a therapeutically effective amount of an immune-therapeutic. In some embodiments, the immune-therapeutic comprises an immune checkpoint inhibitor. In some embodiments, the checkpoint inhibitor comprises PD-1 inhibitors, PD-L1 inhibitors, or CTLA-4 inhibitors, or combinations thereof.

In some embodiments, patients are treated with a therapeutically effective amount of a targeted therapeutic agent. In some embodiments, the targeted therapeutic agent is a drug that targets blood vessel that targets vascular endothelial growth factor (VEGF) such as bevacizumab, ramucirumab, and ziv-aflibercept. In some embodiments, the targeted therapeutic agent comprises an epidermal growth factor receptor (EGFR). In some embodiments, the EGFR comprises cetuximab or panitumumab. In some embodiments, the targeted therapeutic agent comprises a kinase inhibitor. In some embodiments, the kinase inhibitor comprises regorafenib.

In some embodiments, the patient is treated with a targeted therapy. In some embodiments, the methods herein include administering a therapeutically effective amount of one or more of 5-fluorouracil (5-FU); capecitabine, irinotecan, oxaliplatin, trifluridine, or tipiracil.

### Methods for Detecting Glycopeptides

In some embodiments, provided herein are methods for detecting one or more a multiple-reaction-monitoring (MRM) transition, comprising: obtaining a biological sample from a patient, wherein the biological sample comprises one or more glycopeptides; digesting and/or fragmenting a glycopeptide in the sample; and detecting a multiple-reaction-monitoring (MR) transition.

In some embodiments, provided herein are methods of detecting one or more glycopeptides, wherein each glycopeptide is individually in each instance selected from a glycopeptide consisting of an amino acid sequence selected from the group consisting of SEQ ID NOs: 570-595, 673-703, and 731-779, and combinations thereof. In some embodiments, provided herein are methods of detecting one or more glycopeptides, wherein each glycopeptide is individually in each instance selected from a glycopeptide consisting essentially of an amino acid sequence selected from the group consisting of SEQ ID NO: 673-703, 731-779, and 570-595, and combinations thereof.

In some embodiments, provided herein are methods of detecting one or more glycopeptides. In some examples, set forth herein is a method of detecting one or more glycopeptide fragments. In certain examples, the method includes detecting the glycopeptide group to which the glycopeptide, or fragment thereof, belongs. In some of these examples, the glycopeptide group is selected from Alpha-1-antitrypsin (AlAT), Alpha-1B-glycoprotein (A1BG), Leucine-richAlpha-2-glycoprotein (A2GL), Alpha-2-macroglobulin (A2MG),

Alpha-1-antichymotrypsin (AACT), Afamin (AFAM), Alpha-1-acid glycoprotein 1 & 2 (AGP12), Alpha-1-acid glycoprotein 1 (AGP1), Alpha-1-acid glycoprotein 2 (AGP2), Apolipoprotein A-1 (APOA1), Apolipoprotein C-III (APOC3), Apolipoprotein B-100 (APOB), Apolipoprotein D (APOD), Beta-2-glycoprotein-1 (APOH), Apolipoprotein M (APOM), Attractin (ATRN), Calpain-3 (CAN3), Ceruloplasmin (CERU), Complement Factor H (CFAH), Complement Factor I (CFAI), Clusterin (CLUS), ComplementC3 (CO3), ComplementC4-A&B (CO4A&CO4B), ComplementcomponentC6 (CO6), ComplementComponentC8AChain (CO8A), Coagulation factor XII (FA12), Alpha-2-HS-glycoprotein (FETUA), Haptoglobin (HPT), Histidine-rich Glycoprotein (HRG), Immunoglobulin heavy constant alpha 1&2 (IgAl2), Immunoglobulin heavy constant alpha 2 (IgA2), Immunoglobulin heavy constant gamma 2 (IgG2), Immunoglobulin heavy constant mu (IgM), Inter-alpha-trypsin inhibitor heavy chain H1 (ITIH1), Plasma Kallikrein (KLKB1), Kininogen-1 (KNG1), Serum paraoxonase/arylesterase 1 (PON1), Selenoprotein P (SEPP1), Prothrombin (THRB), Serotransferrin (TRFE), Transthyretin (TTR), Protein unc-13HomologA (UN13A), Vitronectin (VTNC), Zinc-alpha-2-glycoprotein (ZA2G), Insulin-like growth factor-II (IGF2), Apolipoprotein C-I (APOC1), and combinations thereof.

In some embodiments, provided herein are methods comprising detecting a glycopeptide, a glycan on the glycopeptide and the glycosylation site residue where the glycan bonds to the glycopeptide. In some embodiments, the method includes detecting a glycan residue. In some embodiments, the method includes detecting a glycosylation site on a glycopeptide. In some embodiments, this process is accomplished with mass spectroscopy used in tandem with liquid chromatography.

In some embodiments, provided herein are methods comprising obtaining a biological sample from a patient. In some examples, the biological sample is synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, bone marrow, gastric acid, bile, semen, pus, aqueous humor, transudate, or combinations of the foregoing. In some examples, the biological sample is selected from the group consisting of blood, plasma, saliva, mucus, urine, stool, tissue, sweat, tears, hair, or a combination thereof. In some examples, the biological sample is a blood sample. In some examples, the biological sample is a plasma sample. In some examples, the biological sample is a saliva sample. In some examples, the biological sample is a mucus sample. In some examples, the biological sample is a urine sample. In some examples, the biological sample is a stool sample. In some examples, the biological sample is a sweat sample. In some examples, the biological sample is a tear sample. In some examples, the biological sample is a hair sample.

In some examples, the method comprises digesting and/or fragmenting a glycopeptide in the sample. In some examples, the method includes digesting a glycopeptide in the sample. In some examples, the method includes fragmenting a glycopeptide in the sample. In some examples, the digested or fragmented glycopeptide is analyzed using mass spectroscopy. In some examples, the glycopeptide is digested or fragmented in the solution phase using digestive enzymes. In some examples, the glycopeptide is digested or fragmented in the gaseous phase inside a mass spectrometer, or the instrumentation associated with a mass spectrometer. In some examples, the mass spectroscopy results are analyzed using machine-learning algorithms. In some examples, the mass spectroscopy results are the quantification of the glycopeptides, glycans, peptides, and fragments thereof. In some examples, this quantification is used as an input in a trained model to generate an output probability. The output probability is a probability of being within a given category or classification, e.g., the classification of having cancer or the classification of not having cancer. In some other examples, the output probability is a probability of being within a given category or classification, e.g., the classification of having cancer or the classification of not having cancer. In some examples, the output probability is a probability of being within a given category or classification, e.g., the classification of having an autoimmune disease or the classification of not having an autoimmune disease. In some examples, the output probability is a probability of being within a given category or classification, e.g., the classification of having fibrosis or the classification of not having fibrosis.

In some examples, the mass spectroscopy is performed using multiple reaction monitoring (MRM) mode. In some examples, the mass spectroscopy is performed using qTOF MS in data-dependent acquisition. In some examples, the mass spectroscopy is performed using or MS-only mode.

In some examples, the method comprises introducing the sample, or a portion thereof, into a mass spectrometer. In some examples, the method comprises fragmenting a glycopeptide in the sample after introducing the sample, or a portion thereof, into the mass spectrometer. In some examples, the method includes digesting a glycopeptide in the sample occurs before introducing the sample, or a portion thereof, into the mass spectrometer. In some examples, the method comprises fragmenting a glycopeptide in the sample to provide a glycopeptide ion, a peptide ion, a glycan ion, a glycan adduct ion, or a glycan fragment ion. In some examples, the method comprises digesting and/or fragmenting a glycopeptide in the sample to provide one or more glycopeptides consisting of an amino acid sequence selected from the group consisting of SEQ ID NO: 673-703, 731-779, and 570-595, and combinations thereof. In some examples, the method comprises digesting and/or fragmenting a glycopeptide in the sample to provide one or more glycopeptides consisting essentially of an amino acid sequence selected from the group consisting of SEQ ID NO: 673-703, 731-779, and 570-595, and combinations thereof.

In some examples, the method includes detecting an MRM transition indicative of a glycopeptide or glycan residue, wherein the glycopeptide consists essentially of an amino acid sequence selected from the group consisting of SEQ ID NO: 673-703, 731-779, and 570-595 and combinations thereof. In some examples, the method includes detecting an MRM transition indicative of a glycopeptide consisting essentially of an amino acid sequence selected from the group consisting of SEQ ID NO: 673-703, 731-779, and 570-595 and combinations thereof. In some examples, the method includes detecting more than one MRM transition indicative of a combination of glycopeptides having amino acid sequences selected from a combination of SEQ ID NO: 673-703, 731-779, and 570-595.

In some examples, the method includes detecting a MRM transition indicative of a glycopeptide or glycan residue, wherein the glycopeptide consists essentially of an amino acid sequence selected from the group consisting of SEQ ID NO: 673-703, 731-779, and 570-595, and combinations thereof. In some examples, the method includes detecting a MRM transition indicative of a glycopeptide consisting essentially of an amino acid sequence selected from the group consisting of SEQ ID NO: 101-131, and combinations thereof. In some examples, the method includes detecting a MRM transition indicative of a glycopeptide consisting essentially of an amino acid sequence selected from the group consisting of SEQ ID NO: 159-207, and combinations thereof. In some examples, the method includes detecting a MRM transition indicative of a glycopeptide consisting essentially of an amino acid sequence selected from the group consisting of SEQ ID NO: 570-595, and combinations thereof.

In some examples, the method includes detecting a MRM transition indicative of a glycopeptide or glycan residue, wherein the glycopeptide consists essentially of an amino acid sequence selected from the group consisting of SEQ ID NOs: 221-46, and combinations thereof. In some examples, the method includes detecting a MRM transition indicative of a glycopeptide consisting essentially of an amino acid sequence selected from the group consisting of SEQ ID NOs: 673-703, and combinations thereof.. In some examples, the method includes detecting a MRM transition indicative of a glycopeptide or glycan residue, wherein the glycopeptide consists essentially of an amino acid sequence selected from the group consisting of 159-207.

In some examples, the method comprises performing mass spectroscopy on the biological sample using multiple-reaction-monitoring mass spectroscopy (MR-MS).

In some examples, the method includes digesting a glycoprotein in the sample to provide one or more glycopeptides consisting essentially of an amino acid sequence selected from the group consisting of SEQ ID NO: 673-703, 731-779, and 570-595, and combinations thereof. In some examples, the biological sample is combined with chemical reagents. In some examples, the biological sample is combined with enzymes. In some examples, the enzymes are lipases. In some examples, the enzymes are proteases. In some examples, the enzymes are serine proteases. In some examples, the enzyme is selected from the group consisting of trypsin, chymotrypsin, thrombin, elastase, and subtilisin. In some examples, the enzyme is trypsin. In some examples, the methods comprises contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, the method includes detecting an MRM transition indicative of a glycopeptide or glycan residue, wherein the glycopeptide consisting of, or consisting essentially of, an amino acid sequence selected from the group consisting of SEQ ID NO: 673-703, 731-779, and 570-595 and combinations thereof. In some examples, the method includes detecting an MRM transition indicative of a glycopeptide or glycan residue, wherein the glycopeptide consists essentially of an amino acid sequence selected from the group consisting of SEQ ID NO: 673-703, 731-779, and 570-595 and combinations thereof. In some examples, the method includes detecting an MRM transition indicative of a glycopeptide consisting essentially of an amino acid sequence selected from the group consisting of SEQ ID NO: 673-703, 731-779, and 570-595 and combinations thereof. In some examples, the method includes detecting more than one MRM transition indicative of a combination of glycopeptides having amino acid sequences selected from a combination of SEQ ID NO: 673-703, 731-779, and 570-595.

In some examples, the method includes detecting a MRM transition indicative of a glycopeptide or glycan residue, wherein the glycopeptide consists essentially of an amino acid sequence selected from the group consisting of SEQ ID NO: 673-703, 731-779, and 570-595, and combinations thereof.

In some examples, the method comprises performing mass spectroscopy on the biological sample using multiple-reaction-monitoring mass spectroscopy (MRM-MS).

In some examples, the method comprises digesting a glycopeptide in the sample to provide a glycopeptide consisting of an amino acid sequence selected from the group consisting of SEQ ID NO: 673-703, 731-779, and 570-595, and combinations thereof. In some examples, the biological sample is contacted with one or more chemical reagents. In some examples, the biological sample is contacted with one or more enzymes. In some examples, the enzymes are lipases. In some examples, the enzymes are proteases. In some examples, the enzymes are serine proteases. In some examples, the enzyme is selected from the group consisting of trypsin, chymotrypsin, thrombin, elastase, and subtilisin. In some of these examples, the enzyme is trypsin. In some examples, the methods include contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, the method includes conducting tandem liquid chromatography-mass spectroscopy on the biological sample. In some examples, the method includes multiple-reaction-monitoring mass spectroscopy (MRM-MS) mass spectroscopy on the biological sample. In some examples, the method includes detecting an MRM transition using a triple quadrupole (QQQ) and/or a quadrupole time-of-flight (qTOF) mass spectrometer. In some examples, the method includes detecting an MRM transition using a QQQ mass spectrometer. In some examples, the method includes detecting using a qTOF mass spectrometer. In some examples, a suitable instrument for use with the instant methods is an Agilent 6495B Triple Quadrupole LC/MS. In some examples, the method includes detecting using a QQQ mass spectrometer. In some examples, a suitable instrument for use with the instant methods is an Agilent 6545 L/Q-TOF.

In some examples, the method comprises detecting more than one MRM transition using a QQQ and/or qTOF mass spectrometer. In some examples, the method includes detecting more than one MRM transition using a QQQ mass spectrometer. In some examples, the method includes detecting more than one MRM transition using a qTOF mass spectrometer. In some examples, the method includes detecting more than one MRM transition using a QQQ mass spectrometer.

In some examples, the methods herein include quantifying one or more glycomic parameters of the one or more biological samples comprises employing a coupled chromatography procedure. In some examples, these glycomic parameters include the identification of a glycopeptide group, identification of glycans on the glycopeptide, identification of a glycosylation site, identification of part of an amino acid sequence which the glycopeptide includes. In some examples, the coupled chromatography procedure comprises: performing or effectuating a liquid chromatography-mass spectrometry (LC-MS) operation. In some examples, the coupled chromatography procedure comprises: performing or effectuating a multiple reaction monitoring mass spectrometry (MRM-MS) operation. In some examples, the methods herein include a coupled chromatography procedure which comprises: performing or effectuating a liquid chromatography-mass spectrometry (LC-MS) operation; and effectuating a multiple reaction monitoring mass spectrometry (MRM-MS) operation. In some examples, the methods include training a machine-learning algorithm using one or more glycomic parameters of the one or more biological samples obtained by one or more of a triple quadrupole (QQQ) mass spectrometry operation and/or a quadrupole time-of-flight (qTOF) mass spectrometry operation. In some examples, the methods include training a machine-learning algorithm using one or more glycomic parameters of the one or more biological samples obtained by a triple quadrupole (QQQ) mass spectrometry operation. In some examples, the methods include training a machine-learning algorithm using one or more glycomic parameters of the one or more biological samples obtained by a quadrupole time-of-flight (qTOF) mass spectrometry operation. In some examples, the methods include quantifying one or more glycomic parameters of the one or more biological samples comprises employing one or more of a triple quadrupole (QQQ) mass spectrometry operation and a quadrupole time-of-flight (qTOF) mass spectrometry operation. In some examples, machine-learning algorithms are used to quantify these glycomic parameters. In some examples, including any of the foregoing, the mass spectroscopy is performed using multiple reaction monitoring (MRM) mode. In some examples, the mass spectroscopy is performed using qTOF MS in data-dependent acquisition. In some examples, the mass spectroscopy is performed using or MS-only mode.

In some examples, the method includes detecting one or more MRM transitions indicative of glycans. In some examples, the method comprises quantifying a glycan. In some examples, the method comprises quantifying a first glycan and quantifying a second glycan; and further comprising comparing the quantification of the first glycan with the quantification of the second glycan. In some examples, the method comprises associating the detected glycan with a peptide residue site, whence the glycan was bonded. In some examples, the method comprises generating a glycosylation profile of the sample. In some examples, the method comprises associating the detected glycan with a timepoint.

In some examples, the method includes spatially profiling glycans on a tissue section associated with the sample. In some examples, including any of the foregoing, the method includes spatially profiling glycopeptides on a tissue section associated with the sample. In some examples, the method includes matrix-assisted laser desorption ionization time-of-flight mass spectrometry (MALDI-TOF) mass spectroscopy in combination with the methods herein.

In some examples, the method includes quantifying relative abundance of a glycan and/or a peptide.

In some examples, the method includes normalizing the amount of a glycopeptide by quantifying a glycopeptide consisting essentially of an amino acid sequence selected from the group consisting of SEQ ID NO: 673-703, 731-779, and 570-595, and combinations thereof and comparing that quantification to the amount of another chemical species. In some examples, the method includes normalizing the amount of a peptide by quantifying a glycopeptide consisting of an amino acid sequence selected from the group consisting of SEQ ID NO: 673-703, 731-779, and 570-595, and combinations thereof, and comparing that quantification to the amount of another glycopeptide consisting of an amino acid sequence selected from the group consisting of SEQ ID NO: 673-703, 731-779, and 570-595. In some examples, the method includes normalizing the amount of a peptide by quantifying a glycopeptide consisting essentially of an amino acid sequence selected from the group consisting of SEQ ID NO: 673-703, 731-779, and 570-595, and combinations thereof, and comparing that quantification to the amount of another glycopeptide consisting essentially of an amino acid sequence selected from the group consisting of SEQ ID NO: 673-703, 731-779, and 570-595.

### Methods for Classifying Samples Comprising Glycopeptides

In some embodiment, provided herein are methods for identifying a classification for a sample, the method comprising: quantifying by mass spectroscopy (MS) one or more glycopeptides in a sample wherein the glycopeptides each, individually in each instance, comprises a glycopeptide consisting essentially of an amino acid sequence selected from the group consisting of, or consisting essentially of SEQ ID NO: 673-703, 731-779, and 570-595, and combinations thereof, and inputting the quantification into a trained model to generate a output probability; determining if the output probability is above or below a threshold for a classification; and identifying a classification for the sample based on whether the output probability is above or below a threshold for a classification.

In some examples, provided herein are methods for identifying glycopeptide biomarkers, comprising: obtaining a biological sample from a patient; digesting and/or fragmenting a glycopeptide in the sample; detecting a multiple-reaction-monitoring (MRM) transition; and classifying the glycopeptides based on the MRM transitions detected. In some examples, a machine-learning algorithm is used to train a model using the analyzed the MRM transitions as inputs. In some examples, a machine-learning algorithm is trained using the MRM transitions as a training data set. In some examples, the methods herein include identifying glycopeptides, peptides, and glycans based on their mass spectroscopy relative abundance. In some examples, a machine-learning algorithm or algorithms select and/or identify peaks in a mass spectroscopy spectrum. In some examples, the MS is MRM-MS with a QQQ and/or qTOF mass spectrometer.

In some examples, including any of the foregoing, the mass spectroscopy is performed using multiple reaction monitoring (MRM) mode. In some examples, the mass spectroscopy is performed using qTOF MS in data-dependent acquisition. In some examples, the mass spectroscopy is performed using or MS-only mode.

In some examples, the machine-learning algorithm is selected from the group consisting of a deep learning algorithm, a neural network algorithm, an artificial neural network algorithm, a supervised machine-learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof. In certain examples, the machine-learning algorithm is lasso regression.

In some examples, the method includes classifying a sample as within, or embraced by, a disease classification or a disease severity classification.

In some examples, the classification is identified with 80% confidence, 85% confidence, 90% confidence, 95% confidence, 99% confidence, or 99.9999% confidence.

In some examples, the method includes quantifying by MS the glycopeptide in a sample at a first time point; quantifying by MS the glycopeptide in a sample at a second time point; and comparing the quantification at the first time point with the quantification at the second time point.

In some examples, the method includes quantifying by MS a different glycopeptide in a sample at a third time point; quantifying by MS the different glycopeptide in a sample at a fourth time point; and comparing the quantification at the fourth time point with the quantification at the third time point.

In some examples, the method includes monitoring the health status of a patient.

In some examples, monitoring the health status of a patient includes monitoring the onset and progression of disease in a patient with risk factors such as genetic mutations, as well as detecting cancer recurrence.

In some examples, the method includes diagnosing a patient with a disease or condition based on the quantification. In some examples, the method includes treating the patient with a therapeutically effective amount of a therapeutic agent comprising one or more of a chemotherapeutic, an immunotherapy, a hormone therapy, a targeted therapy, a neoadjuvant therapy, and surgery. In some embodiments, the treatment comprises checkpoint inhibitors. In some examples, the method includes diagnosing an individual with a disease or condition based on the quantification. In some examples, the method includes treating the individual with a therapeutically effective amount of a treatment.

### Methods for Diagnosing Patients

In some examples, provided herein are methods for assessing a patient having a disease or condition, comprising measuring by mass spectroscopy a glycopeptide in a sample from the patient.

In another embodiment, provided herein are methods for assessing a patient having cancer; the method comprising: obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NO: 673-703, 731-779, and 570-595; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and assessing the patient as having cancer based on the classification.

In another embodiment, set forth herein is a method for diagnosing a patient having cancer; the method comprising: inputting the quantification of detected glycopeptides or MRM transitions into a trained model to generate an output probability, determining if the output probability is above or below a threshold for a classification; and identifying a diagnostic classification for the patient based on whether the output probability is above or below a threshold for a classification; and assessing the patient as based on the classification. In some examples, the method includes obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of 21-46, 101-131, and 159-207.

In some examples, set forth herein is a method for assessing a patient having cancer; the method comprising: obtaining a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NO: 673-703, 731-779, and 570-595; analyzing the detected glycopeptides or the MRM transitions to identify a classification; and assessing the patient based on the diagnostic classification.

In some examples, set forth herein is a method for assessing a patient having cancer; the method comprising: analyzing detected or quantified glycopeptides or MRM transitions to identify a classification; and assessing the patient based on the classification. In some examples, the method includes obtaining a biological sample from the patient; and performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NO: 673-703, 731-779, and 570-595.

In some examples, set forth herein is a method for diagnosing, monitoring, or classifying aging in an individual; the method comprising: obtaining a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NO: 673-703, 731-779, and 570-595; analyzing the detected glycopeptides or the MRM transitions to identify a diagnostic classification; and diagnosing, monitoring, or classifying the individual as having an aging classification based on the diagnostic classification.

### Diseases and Conditions

Provided herein are biomarkers for diagnosing a variety of diseases and conditions. In some examples, the diseases and conditions include cancer. In some examples, the diseases and conditions are not limited to cancer.

In some embodiments, cancer refers to a physiological condition in a subject that is typically characterized by unregulated cell growth. Examples of cancer include, but are not limited to, melanoma, carcinoma, lymphoma, blastoma, sarcoma, and leukemia and metastases thereof. The term “metastasis” refers to the transference of disease-producing organisms or of malignant or cancerous cells to other parts of the body by way of the blood or lymphatic vessels or membranous surfaces. Non-limiting examples of such cancers include small-cell lung cancer, non-small cell lung cancer, adenocarcinoma of the lung, squamous carcinoma of the lung, melanoma, squamous cell cancer, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, thyroid cancer, hepatic carcinoma and various types of head and neck cancer. The phrase “stage of disease” refers to the stages of cancer progression referred to as Stage I, II, III, or IV. Stage of disease indicates if metastasis has occurred in the subject.

In some examples, the “patient” described herein is equivalently described as an “individual.” For example, in some methods herein, set forth are biomarkers for monitoring or diagnosing a disease or a condition in an individual. In some of these examples, the individual is not necessarily a patient who has a medical condition in need of therapy.

### Machine-Learning Model

In some examples, the methods herein comprise quantifying one or more glycopeptides consisting essentially of an amino acid sequence selected from the group consisting of SEQ ID NO: 673-703, 731-779, and 570-595 using mass spectroscopy and/or liquid chromatography. In some examples, the quantification results are used as inputs in a trained model. In some examples, the quantification results are classified or categorized with a predictive algorithm based on the absolute amount, relative amount, and/or type of each glycan or glycopeptide quantified in the test sample, wherein the predictive algorithm is trained on corresponding values for each marker obtained from a population of individuals having known diseases or conditions. In some examples, the disease or condition is cancer. In some cases, the disease or condition is melanoma. In some cases, the disease or condition is NSCLC.

In some examples, including any of the foregoing, set forth herein is a method for training a machine-learning algorithm, comprising: providing a first data set of MRM transition signals indicative of a sample comprising a glycopeptide consisting essentially of an amino acid sequence selected from the group consisting of SEQ ID NO: 673-703, 731-779, and 570-595; providing a second data set of MRM transition signals indicative of a control sample; and comparing the first data set with the second data set using a machine-learning algorithm.

In some examples, the methods herein include using a sample comprising a glycopeptide consisting of an amino acid sequence selected from the group consisting of SEQ ID NO: 673-703, 731-779, and 570-595 is a sample from a patient having the disease or condition. In some examples, the methods herein include using a sample comprising a glycopeptide consisting essentially of an amino acid sequence selected from the group consisting of SEQ ID NO: 673-703, 731-779, and 570-595 is a sample from a patient having cancer. In some examples, the methods herein include using a control sample, wherein the control sample is a sample from a patient not having the disease or condition.

In some examples, the methods herein include using a sample comprising a glycopeptide consisting essentially of an amino acid sequence selected from the group consisting of SEQ ID NO: 673-703, 731-779, and 570-595, which is a pooled sample from one or more patients having the disease or condition. In some examples, the methods herein include using a control sample, which is a pooled sample from one or more patients not having the disease or condition.

In some examples, the methods include generating machine-learning models trained using mass spectrometry data (e.g., MRM-MS transition signals) from patients having a disease or condition and patients not having a disease or condition. In some examples, the disease or condition is cancer. In some examples, the methods include optimizing the machine-learning models by cross-validation with known standards or other samples. In some examples, the methods include qualifying the performance using the mass spectrometry data to form panels of glycans and glycopeptides with individual sensitivities and specificities. In certain examples, the methods include determining a confidence percent in relation to a diagnosis. In some examples, one to ten glycopeptides consisting essentially of an amino acid sequence selected from the group consisting of SEQ ID NO: 673-703, 731-779, and 570-595 may be useful for diagnosing a patient with the disease or condition with a certain confidence percent. In some examples, ten to fifty glycopeptides consisting essentially of an amino acid sequence selected from the group consisting of SEQ ID NO: 673-703, 731-779, and 570-595 may be useful for diagnosing a patient with the disease or condition with a higher confidence percent.

In some examples, including any of the foregoing, the methods include performing MRM-MS and/or LC-MS on a biological sample. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NO: 673-703, 731-779, and 570-595. In some examples, the methods include comparing, by the computing device, the mass spectra data with the theoretical mass spectra data to generate comparison data indicative of a similarity of each of the plurality of mass spectra to each of the plurality of theoretical target mass spectra associated with a corresponding glycopeptide of the plurality of glycopeptides.

In some examples, machine-learning algorithms are used to determine, by the computing device and based on the MRM-MS data, a distribution of a plurality of characteristic ions in the plurality of mass spectra; and determining, by the computing device and based on the distribution, whether one or more of the plurality of characteristic ions is a glycopeptide ion.

In some examples, the methods herein include training a predictive algorithm. Herein, training the predictive algorithm may refer to supervised learning of a predictive algorithm on the basis of values for one or more glycopeptides consisting of, or consisting essentially of, an amino acid sequence selected from the group consisting of SEQ ID NO: 673-703, 731-779, and 570-595. Training the predictive algorithm may refer to variable selection in a statistical model on the basis of values for one or more glycopeptides consisting essentially of an amino acid sequence selected from the group consisting of SEQ ID NO: 673-703, 731-779, and 570-595. Training a predictive algorithm may for example include determining a weighting vector in feature space for each category, or determining a function or function parameters.

In some examples, the machine-learning algorithm is selected from the group consisting of a deep learning algorithm, a neural network algorithm, an artificial neural network algorithm, a supervised machine-learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof. In certain examples, the machine-learning algorithm is lasso regression.

In some examples, the machine-learning algorithm is LASSO, Ridge Regression, Random Forests, K-nearest Neighbors (KNN), Deep Neural Networks (DNN), and Principal Components Analysis (PCA). In certain examples, DNN's are used to process mass spec data into analysis-ready forms. In some examples, DNN's are used for peak picking from a mass spectra. In some examples, PCA is useful in feature detection.

In some examples, LASSO is used to provide feature selection.

In some examples, machine-learning algorithms are used to quantify peptides from each protein that are representative of the protein abundance. In some examples, this quantification includes quantifying proteins for which glycosylation is not measured.

In some examples, glycopeptide sequences are identified by fragmentation in the mass spectrometer and database search using Byonic software (Protein Metrics Inc).

In some examples, the methods herein include unsupervised learning to detect features of MRMS-MS data that represent known biological quantities, such as protein function or glycan motifs. In certain examples, these features are used as input for classifying by machine-learning. In some examples, the classification is performed using LASSO, Ridge Regression, or Random Forest nature.

In some examples, the methods herein include mapping input data (e.g., MRM transition peaks) to a value (e.g., a scale based on 0-100) before processing the value in an algorithm. For example, after an MRM transition is identified and the peak characterized, the methods herein include assessing the MS scans in an m/z and retention time window around the peak for a given patient. In some examples, the resulting chromatogram is integrated by a machine-learning algorithm that determines the peak start and stop points, and calculates the area bounded by those points and the intensity (height). The resulting integrated value is the abundance, which then feeds into machine-learning and statistical analyses training and data sets.

In some examples, machine-learning output, in one instance, is used as machine-learning input in another instance. For example, in addition to the PCA being used for a classification process, the DNN data processing feeds into PCA and other analyses. This results in at least three levels of algorithmic processing. Other hierarchical structures are contemplated within the scope of the instant disclosure.

In some examples, the methods include comparing the amount of each glycan or glycopeptide quantified in the sample to corresponding reference values for each glycan or glycopeptide in a predictive algorithm. In some examples, the methods include a comparative process by which the amount of a glycan or glycopeptide quantified in the sample is compared to a reference value for the same glycan or glycopeptide using a predictive algorithm. The comparative process may be part of a classification by a predictive algorithm. The comparative process may occur at an abstract level, e.g., in n-dimensional feature space or in a higher dimensional space.

In some examples, the methods herein include classifying a patient's sample based on the amount of each glycan or glycopeptide quantified in the sample with a predictive algorithm. In some examples, the methods include using statistical or machine-learning classification processes by which the amount of a glycan or glycopeptide quantified in the test sample is used to determine a category of health with a predictive algorithm. In some examples, the predictive algorithm is a statistical or machine-learning classification algorithm.

In some examples, classification by a predictive algorithm may include scoring likelihood of a panel of glycan or glycopeptide values belonging to each possible category, and determining the highest-scoring category. Classification by a predictive algorithm may include comparing a panel of marker values to previous observations by means of a distance function. Examples of predictive algorithms suitable for classification include random forests, support vector machines, logistic regression (e.g. multiclass or multinomial logistic regression, and/or algorithms adapted for sparse logistic regression). A wide variety of other predictive algorithms that are suitable for classification may be used, as known to a person skilled in the art.

In some examples, the methods herein include supervised learning of a predictive algorithm on the basis of values for each glycan or glycopeptide obtained from a population of individuals having a disease or condition (e.g., melanoma or NSCLS). In some examples, the methods include variable selection in a statistical model on the basis of values for each glycan or glycopeptide obtained from a population of individuals having the disease or condition. Training a predictive algorithm may for example include determining a weighting vector in feature space for each category, or determining a function or function parameters.

In one embodiment, the reference value is the amount of a glycan or glycopeptide in a sample or samples derived from one individual. Alternatively, the reference value may be derived by pooling data obtained from multiple individuals, and calculating an average (for example, mean or median) amount for a glycan or glycopeptide. Thus, the reference value may reflect the average amount of a glycan or glycopeptide in multiple individuals. Said amounts may be expressed in absolute or relative terms, in the same manner as described herein.

In some examples, the reference value may be derived from the same sample as the sample that is being tested, thus allowing for an appropriate comparison between the two. For example, if the sample is derived from urine, the reference value is also derived from urine. In some examples, if the sample is a blood sample (e.g. a plasma or a serum sample), then the reference value will also be a blood sample (e.g. a plasma sample or a serum sample, as appropriate). When comparing between the sample and the reference value, the way in which the amounts are expressed is matched between the sample and the reference value. Thus, an absolute amount can be compared with an absolute amount, and a relative amount can be compared with a relative amount. Similarly, the way in which the amounts are expressed for classification with the predictive algorithm is matched to the way in which the amounts are expressed for training the predictive algorithm.

When the amounts of the glycan or glycopeptide are determined, the method may comprise comparing the amount of each glycan or glycopeptide to its corresponding reference value. When the cumulative amount of one, some or all the glycan or glycopeptides are determined, the method may comprise comparing the cumulative amount to a corresponding reference value. When the amounts of the glycan or glycopeptides are combined with each other in a formula to form an index value, the index value can be compared to a corresponding reference index value derived in the same manner.

The reference values may be obtained either within (i.e., constituting a step of) or external to the (i.e., not constituting a step of) methods described herein. In some examples, the methods include a step of establishing a reference value for the quantity of the markers. In other examples, the reference values are obtained externally to the method described herein and accessed during the comparison step of the invention.

In certain embodiments, the lasso regression machine-learning model may be a regression model or other classification model that may be evaluated utilizing receiver operating characteristic (ROC) evaluation and/or area under curve (AOC) evaluation. For example, in certain embodiments, as will be further illustrated with respect to FIGS. 73A, 73B, 74A, 74B, the ROC model evaluation may represent a plot of sensitivity rate (e.g., patient likely not responsive) against a plot of specificity rate (patient likely to be responsive) and may be further optimized based on an iterative tuning of hyperparameters of the lasso regression machine-learning model. The trained the lasso regression machine-learning model may be then utilized to predict patient overall survival (OS) and progression-free survival (PFS) patients with metastatic melanoma for various glycopeptide fragments and patients with non-small-cell lung cancer (NSCLC) for various glycopeptide fragments, in accordance with the presently disclosed embodiments.

In some examples, including any of the foregoing, training of a predictive algorithm may be obtained either within (i.e., constituting a step of) or external to (i.e., not constituting a step of) the methods set forth herein. In some examples, the methods include a step of training of a predictive algorithm. In some examples, the predictive algorithm is trained externally to the method herein and accessed during the classification step of the invention. The reference value may be determined by quantifying the amount of a glycan or glycopeptide in a sample obtained from a population of healthy individual(s). The predictive algorithm may be trained by quantifying the amount of a glycan or glycopeptide in a sample obtained from a population of healthy individual(s). As used herein, the term “healthy individual” refers to an individual or group of individuals who are in a healthy state, e.g., patients who have not shown any symptoms of the disease, have not been diagnosed with the disease and/or are not likely to develop the disease. Preferably said healthy individual(s) is not on medication affecting the disease and has not been diagnosed with any other disease. The one or more healthy individuals may have a similar sex, age and body mass index (BMI) as compared with the test individual. The reference value may be determined by quantifying the amount of a glycan or glycopeptide in a sample obtained from a population of individual(s) suffering from the disease. The predictive algorithm may be trained by quantifying the amount of a marker in a sample obtained from a population of individual(s) suffering from the disease. More preferably such individual(s) may have similar sex, age and body mass index (BMI) as compared with the test individual. The reference value may be obtained from a population of individuals suffering from cancer. The predictive algorithm may be trained by quantifying the amount of a glycan or glycopeptide in a sample obtained from a population of individuals suffering from cancer. Once the characteristic glycan or glycopeptide profile of cancer is determined, the profile of markers from a biological sample obtained from an individual may be compared to this reference profile to determine whether the test subject also has cancer. Once the predictive algorithm is trained to classify cancer, the profile of markers from a biological sample obtained from an individual may be classified by the predictive algorithm to determine whether the test subject is also at that particular stage of cancer.

### Kits

In some examples, including any of the foregoing, set forth herein is a kit comprising a glycopeptide standard, a buffer, and one or more glycopeptides consisting of an amino acid sequence selected from the group consisting of SEQ ID NO: 673-703, 731-779, and 570-595.

In some examples, including any of the foregoing, set forth herein is a kit comprising a glycopeptide standard, a buffer, and one or more glycopeptides consisting essentially of an amino acid sequence selected from the group consisting of SEQ ID NO: 673-703, 731-779, and 570-595.

In some examples, set forth herein is a kit comprising a glycopeptide standard, a buffer, and one or more glycopeptides consisting of an amino acid sequence selected from the group consisting of SEQ ID NO: 673-703, 731-779, and 570-595, and combinations thereof. In some examples, set forth herein is a kit comprising a glycopeptide standard, a buffer, and one or more glycopeptides consisting essentially of an amino acid sequence selected from the group consisting of SEQ ID NO: 673-703. In some examples, set forth herein is a kit comprising a glycopeptide standard, a buffer, and one or more glycopeptides consisting essentially of an amino acid sequence selected from the group consisting of SEQ ID NO: 731-779. In some examples, set forth herein is a kit comprising a glycopeptide standard, a buffer, and one or more glycopeptides consisting essentially of an amino acid sequence selected from the group consisting of SEQ ID NO: 570-595.

In some examples, set forth herein is a kit for diagnosing or monitoring cancer in an individual wherein the glycan or glycopeptide profile of a sample from said individual is determined and the measured profile is compared with a profile of a normal patient or a profile of a patient with a family history of cancer. In some examples, the kit comprises one or more glycopeptides consisting of an amino acid sequence selected from the group consisting SEQ ID NO: 673-703, 731-779, and 570-595. In some examples, the kit comprises one or more glycopeptides consisting essentially of an amino acid sequence selected from the group consisting of SEQ ID NO: 673-703, 731-779, and 570-595.

In some examples, set forth herein is a kit comprising the reagents for quantification of the oxidized, nitrated, and/or glycated free adducts derived from glycopeptides.

### Clinical Assays

In some examples, the biomarkers, methods, and/or kits may be used in a clinical setting for diagnosing patients. In some of these examples, the analysis of samples includes the use of internal standards. These standards may include one or more glycopeptides consisting of an amino acid sequence selected from the group consisting of SEQ ID NO: 673-703, 731-779, and 570-595. These standards may include one or more glycopeptides consisting essentially of an amino acid sequence selected from the group consisting of SEQ ID NO: 673-703, 731-779, and 570-595.

In a clinical setting, samples may be prepared (e.g., by digestion) to include one or more glycopeptides consisting of an amino acid sequence selected from the group consisting of SEQ ID NO: 673-703, 731-779, and 570-595. In a clinical setting, samples may be prepared (e.g., by digestion) to include one or more glycopeptides consisting essentially of an amino acid sequence selected from the group consisting of SEQ ID NO: 673-703, 731-779, and 570-595. In some examples, the amount of a glycan or glycopeptide may be assessed by comparing the amount of one or more glycopeptides consisting of an amino acid sequence selected from the group consisting of SEQ ID NO: 673-703, 731-779, and 570-595 to the concentration of another biomarker. In some examples, the amount of a glycan or glycopeptide may be assessed by comparing the amount of one or more glycopeptides consisting essentially of an amino acid sequence selected from the group consisting of SEQ ID NO: 673-703, 731-779, and 570-595 to the concentration of another biomarker.

In some examples, the amount of a glycan or glycopeptide may be assessed by comparing the amount of one or more glycopeptides consisting of an amino acid sequence selected from the group consisting of SEQ ID NOs: 826-955 the amount of one or more glycopeptides consisting of an amino acid sequence selected from the group consisting of SEQ ID NOs: 826-955.

In some examples, the amount of a glycan or glycopeptide may be assessed by comparing the amount of one or more glycopeptides consisting essentially of an amino acid sequence selected from the group consisting of SEQ ID NO: 673-703, 731-779, and 570-595 to the amount of one or more glycopeptides consisting essentially of an amino acid sequence selected from the group consisting of SEQ ID NO: 673-703, 731-779, and 570-595.

In some examples, including any of the foregoing, the kit may include software for computing the normalization of a glycopeptide MRM transition signal.

In some examples, including any of the foregoing, the kit may include software for quantifying the amount of a glycopeptide consisting of, or consisting essentially of, an amino acid sequence selected from the group consisting of SEQ ID NO: 673-703, 731-779, and 570-595. In some examples, including any of the foregoing, the kit may include software for quantifying the relative amount of a glycopeptide consisting of, or consisting essentially of, an amino acid sequence selected from the group consisting of SEQ ID NO: 673-703, 731-779, and 570-595.

In some examples, including any of the foregoing, a trained model is stored on a server which is accessed by a clinician performing a method, set forth herein. In some examples, the clinician inputs the quantification of the MRM transition signals from a patient's sample into a trained model which are stored on a server. In some examples, the server is accessed by the internet, wireless communication, or other digital or telecommunication methods.

In some examples, including any of the foregoing, a trained model is stored on a server which is accessed by a clinician performing a method, set forth herein. In some examples, the clinician inputs the quantification of the glycopeptide or glycopeptides consisting of, or consisting essentially of, an amino acid sequence selected from the group consisting of SEQ ID NO: 673-703, 731-779, and 570-595 from a patient's sample into a trained model which are stored on a server. In some examples, the server is accessed by the internet, wireless communication, or other digital or telecommunication

Individual KM curves may be plotted for the markers relevant in for the disease interest in four files. Hazard ratios and p-values on the plots are representative of the plotted high/low split at median biomarker expression. FIGS. 73A and 73B show progression-free survival (PFS) Kaplan-Meier curves of patients with metastatic melanoma for various glycopeptide fragments. FIGS. 74A and 74B show progression-free survival (PFS) Kaplan-Meier curves of patients with non-small-cell lung cancer (NSCLC) for various glycopeptide fragments. FIGS. 75-100 show overall survival (OS) Kaplan-Meier curves of patients with metastatic melanoma for various glycopeptide fragments of interest for melanoma. FIGS. 101-139 show progression-free survival (PFS) Kaplan-Meier curves of patients with metastatic melanoma for various glycopeptide fragments for melanoma.

### IV. Additional Proteins and Glycopeptides

TABLE 66
Glycopeptides associated with melanoma

| | | | | Link- | |
|---|---|---|---|---|---|
| | | | | ing | |
| | | | | Site | |
| | | | | Pos. | |
| | | | | in | |
| | | | | Pep- | Glycan |
| SEQ | Trans- | Peptide | | tide | Struc- |
| ID | ition | Structure | Peptide | Se- | ture |
| NO: | Number | (PS) NAME | Sequence | quence | GL NO. |
| 673 | 673 | QUANTPEP. | DLLLPQPD | N/A | N/A |
| | | A2GL_DL | LR | | |
| | | LLPQPDLR | | | |
| 674 | 674 | QUANTPEP. | SLDFTELD | N/A | N/A |
| | | ANGT_SLDF | VAAEK | | |
| | | TELDVAAEK | | | |
| 675 | 675 | A1AT_ | QLAHQSNS | 1 | 5412 |
| | | 70_5412 | TNIFFSPV | | |
| | | | SIATAFAM | | |
| | | | LSLGTK | | |
| 676 | 676 | HPT_ | MVSHHNLT | 1 | 5412 |
| | | 184_5412 | TGATLINE | | |
| | | | QWLLTTAK | | |
| 677 | 677 | HPT_ | VVLHPNYS | 3 | 6513 |
| | | 241_6513 | QVDIGLIK | | |
| 678 | 678 | HEMO_ | SWPAVGNC | 2 | 5412 |
| | | 187_5412 | SSALR | | |
| 679 | 679 | IC1_ | VATTVISK | 1 | 1102 |
| | | 48_1102 | | | |
| 680 | 680 | HPT_ | MVSHHNLT | 1 | 6513 |
| | | 184_6513 | TGATLINE | | |
| | | | QWLLTTAK | | |
| 681 | 681 | APOC3_74_ | FSEFWDLD | 1 | N/A |
| | | NONGLYCO- | PEVRPTSA | | |
| | | SYLATED | VAA | | |
| 682 | 682 | IGM_ | GLTFQQNA | 2 | 5500 |
| | | 209_5500 | SSMCVPDQ | | |
| | | | DTAIR | | |
| 683 | 683 | FETUA_ | VCQDCPLL | 1 | 5412 |
| | | 156_5412 | APLNDTR | | |
| 684 | 684 | QUANTPEP. | VNHVTLSQ | 1 | |
| | | B2M_VNHV | PK | | |
| | | TLSQPK | | | |
| 685 | 685 | IC1_ | VLSNNSDA | 3 | 5412 |
| | | 253_5412 | NLELINTW | | |
| | | | VAK | | |
| 686 | 686 | CERU_ | EHEGAIYP | 1 | 5412 |
| | | 138_5412 | DNTTDFQR | | |
| 687 | 687 | IGM_ | GLTFQQNA | 2 | 5501 |
| | | 209_5501 | SSMCVPDQ | | |
| | | | DTAIR | | |
| 688 | 688 | THRB_ | WVLTAAHC | 3 | 5402 |
| | | 416MC_ | LLYPPWDK | | |
| | | 5402 | NFTENDLL | | |
| | | | VR | | |
| 689 | 689 | TRFE_ | QQQHLFGS | 2 | 5412 |
| | | 630_5412 | NVTDCSGN | | |
| | | | FCLFR | | |
| 690 | 690 | FETUA_ | AALAAFNA | 2 | 6501 |
| | | 176_6501 | QNNGSNFQ | | |
| | | | LEEISR | | |
| 691 | 691 | CO5_ | ANISHK | 1 | 5412 |
| | | 741_5412 | | | |
| 692 | 692 | FETUA_ | AALAAFNA | 2 | 5412 |
| | | 176_5412 | QNNGSNFQ | | |
| | | | LEEISR | | |
| 693 | 693 | CFAH_ | ISEENETT | 3 | 5401 |
| | | 911_5401 | CYMGK | | |
| 694 | 694 | IGG1_ | EEQYNSTY | 1 | 4511 |
| | | 297_4511 | R | | |
| 695 | 695 | A2MG_ | IITILEEE | 2 | 5200 |
| | | 247_5200 | MNVSVCGL | | |
| | | | YTYGKPVP | | |
| | | | GHVTVSIC | | |
| | | | R | | |
| 696 | 696 | CERU_ | EHEGAIYP | 1 | 5402 |
| | | 138_5402 | DNTTDFQR | | |
| 697 | 697 | IGA2_ | TPLTANIT | 1 | 4510 |
| | | 205_4510 | K | | |
| 698 | 698 | HRG_ | VIDFNCTT | 1 | 5402 |
| | | 125_5402 | SSVSSALA | | |
| | | | NTK | | |
| 699 | 699 | HPT_207_ | NLFLNHSE | 2 | 121005 |
| | | 121005 | NATAK | | |
| 700 | 700 | AACT_ | FNLTETSE | 1 | 7604 |
| | | 106_7604 | AEIHQSFQ | | |
| | | | HLLR | | |
| 701 | 701 | CERU_ | ENLTAPGS | 3 | 6503 |
| | | 397_6503 | DSAVFFEQ | | |
| | | | GTTR | | |
| 702 | 702 | HPT_ | NLFLNHSE | 2 | 11904 |
| | | 207_11904 | NATAK | | |

TABLE 67
Glycoproteins Associated with Melanoma

| SEQ | Protein | | Uniprot |
|---|---|---|---|
| ID NO. | Abbreviation | Protein Name | ID |
| 704 | A2GL | Leucine-richAlpha- | P02750 |
| | | 2-glycoprotein | |
| 705 | ANGT | P01019\|Angiotensinogen | P01019 |
| 706 | HPT | Haptoglobin | P00738 |
| 707 | HEMO | Hemopexin | P02790 |
| 708 | IC1 | Plasma protease C1 inhibitor | P05155 |
| 709 | HPT | Haptoglobin | P00738 |
| 710 | APOC3 | Apolipoprotein C-III | P02656 |
| 711 | IGM | Immunoglobulin heavy | P01871 |
| | | constant mu | |
| 712 | FETUA | Alpha-2-HS-glycoprotein | P02765 |
| 713 | B2M | Beta-2-microglobulin | P61769 |
| 714 | IC1 | Plasma protease C1 inhibitor | P05155 |
| 715 | CERU | Ceruloplasmin | P00450 |
| 716 | IGM | Immunoglobulin heavy | P01871 |
| | | constant mu | |
| 717 | THRB | Prothrombin | P00734 |
| 718 | TRFE | Serotransferrin | P02787 |
| 719 | FETUA | Alpha-2-HS-glycoprotein | P02765 |
| 720 | CO5 | ComplementC5 | P01031 |
| 721 | FETUA | Alpha-2-HS-glycoprotein | P02765 |
| 722 | CFAH | ComplementFactorH | P08603 |
| 723 | IGG1 | Immunoglobulin heavy | P01857 |
| | | constant gamma 1 | |
| 724 | A2MG | Alpha-2-macroglobulin | P01023 |
| 725 | CERU | Ceruloplasmin | P00450 |
| 726 | IGA2 | Immunoglobulin heavy | P01877 |
| | | constant alpha 2 | |
| 727 | HRG | Histidine-rich Glycoprotein | P04196 |
| 728 | HPT | Haptoglobin | P00738 |
| 729 | AACT | Alpha-1-antichymotrypsin | P01011 |
| 730 | HPT | Haptoglobin | P00738 |

TABLE 68
Glycopeptides Associated with NSCLC

| | | | | Linking | |
|---|---|---|---|---|---|
| SEQ | Trans- | Peptide | | Site Pos. | Glycan |
| ID | ition | Structure | Peptide | in Peptide | Structure |
| NO: | Number | (PS) NAME | Sequence | Sequence | GL NO. |
| 731 | 731 | TRFE_630_6513 | QQQHLFGSNVTDCSG | 9 | 6513 |
| | | | NFCLFR | | |
| 732 | 732 | AGP1_93_6503 | QDQCIYNTTYLNVQR | 7 | 6503 |
| 733 | 733 | IGG2_297_5510 | EEQFNSTFR | 5 | 5510 |
| 734 | 734 | IGG1_297_5410 | EEQYNSTYR | 5 | 5410 |
| 735 | 735 | AACT_271_6502 | YTGNASALFILPDQDK | 4 | 6502 |
| 736 | 736 | AGP1_103_6503 | ENGTISR | 2 | 6503 |
| 737 | 737 | IGG1_297_3410 | EEQYNSTYR | 5 | 3410 |
| 738 | 738 | IGG1_297_5510 | EEQYNSTYR | 5 | 5510 |
| 739 | 739 | VTNC_86_6503 | NNATVHEQVGGPSLT | 2 | 6503 |
| | | | SDLQAQSK | | |
| 740 | 740 | HPT_241_6513 | VVLHPNYSQVDIGLIK | 6 | 6513 |
| 741 | 741 | CERU_762_6523 | ELHHLQEQNVSNAFL | 9 | 6523 |
| | | | DK | | |
| 742 | 742 | HRG_345_5412 | HSHNNNSSDLHPHK | 6 | 5412 |
| 743 | 743 | HPT_207_5401 | NLFLNHSENATAK | 5 | 5401 |
| 744 | 744 | AGP1_93_8704 | QDQCIYNTTYLNVQR | 7 | 8704 |
| 745 | 745 | HRG_125_5402 | VIDFNCTTSSVSSALA | 5 | 5402 |
| | | | NTK | | |
| 746 | 746 | A1AT_271_5401 | YLGNATAIFFLPDEGK | 4 | 5401 |
| 747 | 747 | KNG1_205_5412 | ITYSIVQTNCSK | 9 | 5412 |
| 748 | 748 | TRFE_432_5401 | CGLVPVLAENYNK | 12 | 5401 |
| 749 | 749 | IGG2_297_5410 | EEQFNSTFR | 5 | 5410 |
| 750 | 750 | TRFE_630_5400 | QQQHLFGSNVTDCSG | 9 | 5400 |
| | | | NFCLFR | | |
| 751 | 751 | AGP1_93_7603 | QDQCIYNTTYLNVQR | 7 | 7603 |
| 752 | 752 | CERU_762_6512 | ELHHLQEQNVSNAFL | 9 | 6512 |
| | | | DK | | |
| 753 | 753 | A1AT_107_6502 | ADTHDEILEGLNFNLT | 14 | 6502 |
| | | | EIPEAQIHEGFQELLR | | |
| 754 | 754 | KLKB1_494_5400 | LQAPLNYTEFQKPICL | 6 | 5400 |
| | | | PSK | | |
| 755 | 755 | IGG1_297_5411 | EEQYNSTYR | 5 | 5411 |
| 756 | 756 | HPT_207_121005 | NLFLNHSENATAK | 5, 9 | 121005 |
| 757 | 757 | FETUA_176_5412 | AALAAFNAQNNGSNF | 11 | 5412 |
| | | | QLEEISR | | |
| 758 | 758 | HPT_241_5412 | VVLHPNYSQVDIGLIK | 6 | 5412 |
| 759 | 759 | CFAH_882_5401 | IPCSQPPQIEHGTINSSR | 15 | 5401 |
| 760 | 760 | AGP1_93_6502 | QDQCIYNTTYLNVQR | 7 | 6502 |
| 761 | 761 | IC1_352_5412 | VGQLQLSHNLSLVILV | 9 | 5412 |
| | | | PQNLK | | |
| 762 | 762 | HEMO_187_NON- | SWPAVGNCSSALR | 7 | NONGLY- |
| | | GLYCOSYLATED | | | COSYL- |
| | | | | | ATED |
| 763 | 763 | KLKB1_396_5401 | IVGGTNSSWGEWPWQ | 6 | 5401 |
| | | | VSLQVK | | |
| 764 | 764 | IGJ_71_5412 | ENISDPTSPLR | 2 | 5412 |
| 765 | 765 | AGP12_72MC_ | SVQEIQATFFYFTPNK | 15 | 7614 |
| | | 7614 | TEDTIFLR | | |
| 766 | 766 | TRFE_630_5401 | QQQHLFGSNVTDCSG | 9 | 5401 |
| | | | NFCLFR | | |
| 767 | 767 | TRFE_630_5411 | QQQHLFGSNVTDCSG | 9 | 5411 |
| | | | NFCLFR | | |
| 768 | 768 | IGM_209_5512 | GLTFQQNASSMCVPD | 7 | 5512 |
| | | | QDTAIR | | |
| 769 | 769 | KNG1_137_ | FSVATQTCQITPAEGP | N/A | NONGLY- |
| | | NONGLYCO- | VVTAQYDCLGCVHPIS | | COSYL- |
| | | SYLATED | TQSPDLEPILR | | ATED |
| 770 | 770 | FHR1_126_5402 | LQNNENNISCVER | 7 | 5402 |
| 771 | 771 | IGG1_297_4500 | EEQYNSTYR | 5 | 4500 |
| 772 | 772 | AGP1_93_7612 | QDQCIYNTTYLNVQR | 7 | 7612 |
| 773 | 773 | A1AT_271_5402 | YLGNATAIFFLPDEGK | 4 | 5402 |
| 774 | 774 | A1AT_271_6503 | YLGNATAIFFLPDEGK | 4 | 6503 |
| 775 | 775 | KNG1_294_5412 | LNAENNATFYFK | 6 | 5412 |
| 776 | 776 | CO2_621_6200 | QSVPAHFVALNGSK | 11 | 6200 |
| 777 | 777 | HRG_271_2202 | SSTTKPPFKPHGSR | 1 | 2202 |
| 778 | 778 | APOD_98_5412 | ADGTVNQIEGEATPVN | 16 | 5412 |
| | | | LTEPAK | | |
| 779 | 779 | AFAM_33_5402 | DIENFNSTQK | 6 | 5402 |

TABLE 69
Glycoproteins associated with NSCLC

| SEQ | Protein | | |
|---|---|---|---|
| ID NO. | Abbreviation | Protein Name | Uniprot ID |
| 780 | TRFE | Serotransferrin | P02787 |
| 781 | AGP1 | Alpha-1-acid glycoprotein 1 | P02763 |
| 782 | IGG2 | Immunoglobulin heavy | P01859 |
| | | constant gamma 2 | |
| 783 | IGG1 | Immunoglobulin heavy | P01857 |
| | | constant gamma 1 | |
| 784 | AACT | Alpha-1-antichymotrypsin | P01011 |
| 785 | AGP1 | Alpha-1-acid glycoprotein 1 | P02763 |
| 786 | IGG1 | Immunoglobulin heavy | P01857 |
| | | constant gamma 1 | |
| 787 | VTNC | Vitronectin | P04004 |
| 788 | HPT | Haptoglobin | P00738 |
| 789 | CERU | Ceruloplasmin | P00450 |
| 790 | HRG | Histidine-rich Glycoprotein | P04196 |
| 791 | HPT | Haptoglobin | P00738 |
| 792 | AGP1 | Alpha-1-acid glycoprotein 1 | P02763 |
| 793 | HRG | Histidine-rich Glycoprotein | P04196 |
| 794 | A1AT | Alpha-1-antitrypsin | P01009 |
| 795 | KNG1 | Kininogen-1 | P01042 |
| 796 | TRFE | Serotransferrin | P02787 |
| 797 | IGG2 | Immunoglobulin heavy | P01859 |
| | | constant gamma 2 | |
| 798 | TRFE | Serotransferrin | P02787 |
| 799 | AGP1 | Alpha-1-acid glycoprotein 1 | P02763 |
| 800 | CERU | Ceruloplasmin | P00450 |
| 801 | A1AT | Alpha-1-antitrypsin | P01009 |
| 802 | KLKB1 | Plasma Kallikrein | P03952 |
| 803 | IGG1 | Immunoglobulin heavy | P01857 |
| | | constant gamma 1 | |
| 804 | HPT | Haptoglobin | P00738 |
| 805 | FETUA | Alpha-2-HS-glycoprotein | P02765 |
| 806 | HPT | Haptoglobin | P00738 |
| 807 | CFAH | Complement Factor H | P08603 |
| 808 | AGP1 | Alpha-1-acid glycoprotein 1 | P02763 |
| 809 | IC1 | Plasma protease C1 inhibitor | P05155 |
| 810 | HEMO | Hemopexin | P02790 |
| 811 | KLKB1 | Plasma Kallikrein | P03952 |
| 812 | IGJ | Immunoglobulin J chain | P01591 |
| 813 | AGP1&2 | Alpha-1-acid | P02763&P19652 |
| | | glycoprotein 1&2 | |
| 814 | TRFE | Serotransferrin | P02787 |
| 815 | IGM | Immunoglobulin heavy | P01871 |
| | | constant mu | |
| 816 | KNG1 | Kininogen-1 | P01042 |
| 817 | FHR1 | Complement factor | Q03591 |
| | | H-related protein 1 | |
| 818 | IGG1 | Immunoglobulin heavy | P01857 |
| | | constant gamma 1 | |
| 819 | AGP1 | Alpha-1-acid glycoprotein 1 | P02763 |
| 820 | A1AT | Alpha-1-antitrypsin\|A1AT | P01009 |
| 821 | KNG1 | Kininogen-1\|KNG1 | P01042 |
| 822 | CO2 | ComplementC2 | P06681 |
| 823 | HRG | Histidine-rich Glycoprotein | P04196 |
| 824 | APOD | Apolipoprotein D | P05090 |
| 825 | AFAM | Afamin | P43652 |

TABLE 70
Glycopeptides

| | | | | | Linking | |
|---|---|---|---|---|---|---|
| Pept | | | | | Site | |
| SEQ | Peptide | Protein | Prot | | Pos. in | Glycan |
| ID | Structure | Abbrevi- | SEQ | Peptide | Protein | Structure |
| NO: | (PS) Name | ation | ID | Sequence | Sequence | GL NO. |
| 826 | A1AT_70_5412 | A1AT | 956 | QLAHQSNSTNIFFSP | 70 | 5412 |
| | | | | VSIATAFAMLSLGT | | |
| | | | | K | | |
| 827 | AFAM_33_5402 | AFAM | 991 | DIENFNSTQK | 33 | 5402 |
| 828 | AGP1_93_6502 | AGP1 | 975 | QDQCIYNTTYLNVQ | 93 | 6502 |
| | | | | R | | |
| 829 | AGP12_72MC_ | AGP1 | 975 | SVQEIQATFFYFTPN | 72 | 7614 |
| | 7614 | AGP2 | 996 | KTEDTIFLR | | |
| 830 | CFAH_911_540 | CFAH | 987 | ISEENETTCYMGK | 911 | 5401 |
| | 1 | | | | | |
| 831 | HPT_207_10803 | HPT | 962 | NLFLNHSENATAK | 207 | 10803 |
| 832 | HPT_207_5401 | HPT | 962 | NLFLNHSENATAK | 207 | 5401 |
| 833 | HPT_241_5412 | HPT | 962 | VVLHPNYSQVDIGLI | 241 | 5412 |
| | | | | K | | |
| 834 | IGG1_297_5400 | IGG1 | 969 | EEQYNSTYR | 297 | 5400 |
| 835 | TRFE_630_5400 | TRFE | 977 | QQQHLFGSNVTDCS | 630 | 5400 |
| | | | | GNFCLFR | | |
| 836 | AGP1_33_6503 | AGP1 | 975 | QIPLCANLVPVPITN | 33 | 6503 |
| | | | | ATLDQITGK | | |
| 837 | UN13A_1005_7 | UN13A | 995 | ACLNSTYEYIFNNC | 1005 | 7512 |
| | 512 | | | HELYSR | | |
| 838 | A1AT_107_650 | A1AT | 956 | ADTHDEILEGLNFN | 107 | 6502 |
| | 2 | | | LTEIPEAQIHEGFQE | | |
| | | | | LLR | | |
| 839 | A1AT_271_540 | A1AT | 956 | YLGNATAIFFLPDEG | 271 | 5401 |
| | 1 | | | K | | |
| 840 | A1AT_271_540 | A1AT | 956 | YLGNATAIFFLPDEG | 271 | 5402 |
| | 2 | | | K | | |
| 841 | A1AT_271_650 | A1AT | 956 | YLGNATAIFFLPDEG | 271 | 6503 |
| | 3 | | | K | | |
| 842 | A2MG_247_520 | A2MG | 965 | IITILEEEMNVSVCG | 247 | 5200 |
| | 0 | | | LYTYGKPVPGHVTV | | |
| | | | | SICR | | |
| 843 | A2MG_869_520 | A2MG | 965 | SLGNVNFTVSAEAL | 869 | 5200 |
| | 0 | | | ESQELCGTEVPSVPE | | |
| | | | | HGR | | |
| 844 | A2MG_869_620 | A2MG | 965 | SLGNVNFTVSAEAL | 869 | 6200 |
| | 0 | | | ESQELCGTEVPSVPE | | |
| | | | | HGR | | |
| 845 | AACT_106_760 | AACT | 963 | FNLTETSEAEIHQSF | 106 | 7604 |
| | 4 | | | QHLLR | | |
| 846 | AACT_127_540 | AACT | 963 | TLNQSSDELQLSMG | 127 | 5401 |
| | 1 | | | NAMFVK | | |
| 847 | AACT_271_650 | AACT | 963 | YTGNASALFILPDQ | 271 | 6502 |
| | 2 | | | DK | | |
| 848 | AACT_271_760 | AACT | 963 | YTGNASALFILPDQ | 271 | 7602 |
| | 2 | | | DK | | |
| 849 | AACT_271_760 | AACT | 963 | YTGNASALFILPDQ | 271 | 7603 |
| | 3 | | | DK | | |
| 850 | AGP1_103_650 | AGP1 | 975 | ENGTISR | 103 | 6503 |
| | 3 | | | | | |
| 851 | AGP1_33_6502 | AGP1 | 975 | QIPLCANLVPVPITN | 33 | 6502 |
| | | | | ATLDQITGK | | |
| 852 | AGP1_33_6503 | AGP1 | 975 | QIPLCANLVPVPITN | 33 | 6503 |
| | | | | ATLDQITGK | | |
| 853 | AGP1_93_6503 | AGP1 | 975 | QDQCIYNTTYLNVQ | 93 | 6503 |
| | | | | R | | |
| 854 | AGP1_93_7603 | AGP1 | 975 | QDQCIYNTTYLNVQ | 93 | 7603 |
| | | | | R | | |
| 855 | AGP1_93_7612 | AGP1 | 975 | QDQCIYNTTYLNVQ | 93 | 7612 |
| | | | | R | | |
| 856 | AGP1_93_8704 | AGP1 | 975 | QDQCIYNTTYLNVQ | 93 | 8704 |
| | | | | R | | |
| 857 | APOC3_74_ | APOC3 | 973 | FSEFWDLDPEVRPTS | 74 | NON- |
| | NON-GLYCO- | | | AVAA | | GLYCO- |
| | SYLATED | | | | | SYLATED |
| 858 | APOD_98_5412 | APOD | 982 | ADGTVNQIEGEATP | 98 | 5412 |
| | | | | VNLTEPAK | | |
| 859 | CERU_138_540 | CERU | 960 | EHEGAIYPDNTTDF | 138 | 5402 |
| | 2 | | | QR | | |
| 860 | CERU_138_541 | CERU | 960 | EHEGAIYPDNTTDF | 138 | 5412 |
| | 2 | | | QR | | |
| 861 | CERU_397_650 | CERU | 960 | ENLTAPGSDSAVFFE | 397 | 6503 |
| | 3 | | | QGTTR | | |
| 862 | CERU_762_651 | CERU | 960 | ELHHLQEQNVSNAF | 762 | 6512 |
| | 2 | | | LDK | | |
| 863 | CERU_762_652 | CERU | 960 | ELHHLQEQNVSNAF | 762 | 6523 |
| | 3 | | | LDK | | |
| 864 | CFAH_1029_54 | CFAH | 987 | MDGASNVTCINSR | 1029 | 5401 |
| | 01 | | | | | |
| 865 | CFAH_1029_54 | CFAH | 987 | MDGASNVTCINSR | 1029 | 5402 |
| | 02 | | | | | |
| 866 | CFAH_882_540 | CFAH | 987 | IPCSQPPQIEHGTINS | 882 | 5401 |
| | 1 | | | SR | | |
| 867 | CLUS_374_650 | CLUS | 988 | LANLTQGEDQYYLR | 374 | 6501 |
| | 1 | | | | | |
| 868 | CO2_621_6200 | CO2 | 985 | QSVPAHFVALNGSK | 621 | 6200 |
| 869 | CO5_741_5412 | CO5 | 966 | ANISHK | 741 | 5412 |
| 870 | CO8B_243_661 | CO8B | 986 | EYESYSDFERNVTE | 243 | 6610 |
| | 0 | | | K | | |
| 871 | CO8B_553_541 | CO8B | 986 | WNCWSNWSSCSGR | 553 | 5410 |
| | 0 | | | | | |
| 872 | FETUA_156_54 | FETUA | 976 | VCQDCPLLAPLNDT | 156 | 5402 |
| | 02 | | | R | | |
| 873 | FETUA_156_54 | FETUA | 976 | VCQDCPLLAPLNDT | 156 | 5412 |
| | 12 | | | R | | |
| 874 | FETUA_176_54 | FETUA | 976 | AALAAFNAQNNGS | 176 | 5412 |
| | 12 | | | NFQLEEISR | | |
| 875 | FETUA_176_65 | FETUA | 976 | AALAAFNAQNNGS | 176 | 6501 |
| | 01 | | | NFQLEEISR | | |
| 876 | FHR1_126_5402 | FHR1 | 993 | LQNNENNISCVER | 126 | 5402 |
| 877 | HEMO_187_541 | HEMO | 978 | SWPAVGNCSSALR | 187 | 5412 |
| | 2 | | | | | |
| 878 | HEMO_187_ | HEMO | 978 | SWPAVGNCSSALR | 187 | NON- |
| | NON-GLYCO- | | | | | GLYCO- |
| | SYLATED | | | | | SYLATED |
| 879 | HEMO_453_540 | HEMO | 978 | ALPQPQNVTSLLGC | 453 | 5401 |
| | 1 | | | TH | | |
| 880 | HPT_184_5412 | HPT | 962 | MVSHHNLTTGATLI | 184 | 5412 |
| | | | | NEQWLLTTAK | | |
| 881 | HPT_184_5511 | HPT | 962 | MVSHHNLTTGATLI | 184 | 5511 |
| | | | | NEQWLLTTAK | | |
| 882 | HPT_184_6513 | HPT | 962 | MVSHHNLTTGATLI | 184 | 6513 |
| | | | | NEQWLLTTAK | | |
| 883 | HPT_207_11904 | HPT | 962 | NLFLNHSENATAK | 207 | 11904 |
| 884 | HPT_207_12100 | HPT | 962 | NLFLNHSENATAK | 207 | 121005 |
| | 5 | | | | | |
| 885 | HPT_241_5401 | HPT | 962 | VVLHPNYSQVDIGLI | 241 | 5401 |
| | | | | K | | |
| 886 | HPT_241_6513 | HPT | 962 | VVLHPNYSQVDIGLI | 241 | 6513 |
| | | | | K | | |
| 887 | HR_ 125_5401 | HRG | 981 | VIDFNCTTSSVSSAL | 125 | 5401 |
| | | | | ANTK | | |
| 888 | HRG_125_5402 | HRG | 98 | VIDFNCTTSSVSSAL | 125 | 5402 |
| | | | | ANTK | | |
| 889 | HRG_271_2202 | HRG | 981 | SSTTKPPFKPHGSR | 271 | 2202 |
| 890 | HRG_345_5412 | HRG | 981 | HSHNNNSSDLHPHK | 345 | 5412 |
| 891 | IC1_253_5412 | IC1 | 983 | VLSNNSDANLELINT | 253 | 5412 |
| | | | | WVAK | | |
| 892 | IC1_352_5412 | IC1 | 983 | VGQLQLSHNLSL VIL | 352 | 5412 |
| | | | | VPQNLK | | |
| 893 | IC1_48_1102 | IC1 | 983 | VATTVISK | 48 | 1102 |
| 894 | IGA12_144_350 | IGA1 | 555 | LSLHRPALEDLLLGS | 144 | 3500 |
| | 0 | IGA2 | 972 | EANLTCTLTGLR | | |
| 895 | IGA12_144_440 | IGA1 | 555 | LSLHRPALEDLLLGS | 144 | 4401 |
| | 1 | IGA2 | 972 | EANLTCTLTGLR | | |
| 896 | IGA12_144_450 | IGA1 | 555 | LSLHRPALEDLLLGS | 144 | 4500 |
| | 0 | IGA2 | 972 | EANLTCTLTGLR | | |
| 897 | IGA12_144_550 | IGA1 | 555 | LSLHRPALEDLLLGS | 144 | 5501 |
| | 1 | IGA2 | 972 | EANLTCTLTGLR | | |
| 898 | IGA12_144_550 | IGA1 | 555 | LSLHRPALEDLLLGS | 144 | 5502 |
| | 2 | IGA2 | 972 | EANLTCTLTGLR | | |
| 899 | IGA2_205_4510 | IGA2 | 972 | TPLTANITK | 205 | 4510 |
| 900 | IGG1_297_3410 | IGG1 | 969 | EEQYNSTYR | 297 | 3410 |
| 901 | IGG1_297_4400 | IGG1 | 969 | EEQYNSTYR | 297 | 4400 |
| 902 | IGG1_297_4500 | IGG1 | 969 | EEQYNSTYR | 297 | 4500 |
| 903 | IGG1_297_4510 | IGG1 | 969 | EEQYNSTYR | 297 | 4510 |
| 904 | IGG1_297_4511 | IGG1 | 969 | EEQYNSTYR | 297 | 4511 |
| 905 | IGG1_297_5410 | IGG1 | 969 | EEQYNSTYR | 297 | 5410 |
| 906 | IGG1_297_5411 | IGG1 | 969 | EEQYNSTYR | 297 | 5411 |
| 907 | IGG1_297_5510 | IGG1 | 969 | EEQYNSTYR | 297 | 5510 |
| 908 | IGG2_297_4411 | IGG2 | 782 | EEQFNSTFR | 297 | 4411 |
| 909 | IGG2_297_4500 | IGG2 | 782 | EEQFNSTFR | 297 | 4500 |
| 910 | IGG2_297_5410 | IGG2 | 782 | EEQFNSTFR | 297 | 5410 |
| 911 | IGG2_297_5411 | IGG2 | 782 | EEQFNSTFR | 297 | 5411 |
| 912 | IGG2_297_5510 | IGG2 | 782 | EEQFNSTFR | 297 | 5510 |
| 913 | IGG34_297_441 | IGG3 | 563 | EEQYNSTFR | 297 | 4410 |
| | 0 | IGG4 | 970 | | | |
| 914 | IGG34_297_441 | IGG3 | 563 | EEQYNSTFR | 297 | 4411 |
| | 1 | IGG4 | 970 | | | |
| 915 | IGJ_71_5412 | IGJ | 968 | ENISDPTSPLR | 71 | 5412 |
| 916 | IGM_209_5500 | IGM | 971 | GLTFQQNASSMCVP | 209 | 5500 |
| | | | | DQDTAIR | | |
| 917 | IGM_209_5501 | IGM | 97 | GLTFQQNASSMCVP | 209 | 5501 |
| | | | | DQDTAIR | | |
| 918 | IGM_209_5510 | IGM | 971 | GLTFQQNASSMCVP | 209 | 5510 |
| | | | | DQDTAIR | | |
| 919 | IGM_209_5512 | IGM | 971 | GLTFQQNASSMCVP | 209 | 5512 |
| | | | | DQDTAIR | | |
| 920 | ITIH4_517_540 | ITIH4 | 994 | LPTQNITFQTESSVA | 517 | 5401 |
| | 1 | | | EQEAEFQSPK | | |
| 921 | KLKB1_127_54 | KLKB1 | 979 | GVNFNVSK | 127 | 5410 |
| | 10 | | | | | |
| 922 | KLKB1_396_54 | KLKB1 | 979 | IVGGTNSSWGEWP | 396 | 5401 |
| | 01 | | | WQVSLQVK | | |
| 923 | KLKB1_494_54 | KLKB1 | 979 | LQAPLNYTEFQKPIC | 494 | 5400 |
| | 00 | | | LPSK | | |
| 924 | KLKB1_494_54 | KLKB1 | 979 | LQAPLNYTEFQKPIC | 494 | 5410 |
| | 10 | | | LPSK | | |
| 925 | KNG1_137_ | KNG1 | 967 | FSVATQTCQITPAEG | 137 | NON- |
| | NONGLYCO- | | | PVVTAQYDCLGCV | | GLYCO- |
| | SYLATED | | | HPISTQSPDLEPILR | | SYLATED |
| 926 | KNG1_205_541 | KNG1 | 967 | ITYSIVQTNCSK | 205 | 5412 |
| | 2 | | | | | |
| 927 | KNG1_294_541 | KNG1 | 967 | LNAENNATFYFK | 294 | 5412 |
| | 2 | | | | | |
| 928 | NEWQUANTPE | IGG3 | 540 | TPEVTCVVVDVSHE | N/A | N/A |
| | P- | | | DPEVQFK | | |
| | IGG3_TPEVTC | | | | | |
| | VVVDVSHEDP | | | | | |
| | EVQFK | | | | | |
| 929 | QUANTPEP.A1 | A1AT | 956 | AVLTIDEK | N/A | N/A |
| | AT_AVLTIDEK | | | | | |
| 930 | QUANTPEP.A2 | A2GL | 974 | DLLLPQPDLR | N/A | N/A |
| | GL_DLLLPQPD | | | | | |
| | LR | | | | | |
| 931 | QUANTPEP.AN | ANGT | 964 | SLDFTELDVAAEK | N/A | N/A |
| | GT_SLDFTELD | | | | | |
| | VAAEK | | | | | |
| 932 | QUANTPEP.B2 | B2M | 992 | VNHVTLSQPK | N/A | N/A |
| | M_VNHVTLSQ | | | | | |
| | PK | | | | | |
| 933 | QUANTPEP.IG | IGG4 | 970 | TTPPVLDSDGSFFLY | N/A | N/A |
| | G4_TTPPVLDS | | | SR | | |
| | DGSFFLYSR | | | | | |
| 934 | QUANTPEP.TR | TFRE | 977 | DDTVCLAK | N/A | N/A |
| | FE_DDTVCLA | | | | | |
| | K | | | | | |
| 935 | THBG_36_5402 | THBG | 984 | VTACHSSQPNATLY | 36 | 5402 |
| | THBG | | | K | | |
| 936 | THRB_416MC_ | THRB | 959 | WVLTAAHCLL YPP | 416 | 5402 |
| | 5402 | | | WDKNFTENDLL VR | | |
| 937 | TRFE_432_5401 | TRFE | 977 | CGLVPVLAENYNK | 432 | 5401 |
| 938 | TRFE_630_5401 | TRFE | 977 | QQQHLFGSNVTDCS | 630 | 5401 |
| | | | | GNFCLFR | | |
| 939 | TRFE_630_5411 | TRFE | 977 | QQQHLFGSNVTDCS | 630 | 5411 |
| | | | | GNFCLFR | | |
| 940 | TRFE_630_5412 | TRFE | 977 | QQQHLFGSNVTDCS | 630 | 5412 |
| | | | | GNFCLFR | | |
| 941 | TRFE_630_6513 | TRFE | 977 | QQQHLFGSNVTDCS | 630 | 6513 |
| | | | | GNFCLFR | | |
| 942 | VTNC_169_540 | VTNC | 980 | NGSLFAFR | 169 | 5401 |
| | 1 | | | | | |
| 943 | VTNC_86_6503 | VTNC | 980 | NNATVHEQVGGPSL | 86 | 6503 |
| | | | | TSDLQAQSK | | |
| 944 | PON1_324_650 | PON1 | 990 | VTQVYAENGTVLQ | 324 | 6501 |
| | 1 | | | GSTVASVYK | | |
| 945 | UN13A_1005_5 | UN13A | 995 | ACLNSTYEYIFNNC | 1005 | 5431 |
| | 431 | | | HEL YSR | | |
| 946 | CAN3_366_651 | CAN3 | 989 | NPWGQVEWNGSWS | 366 | 6513 |
| | 3 | | | DR | | |
| 947 | UN13A_1005_7 | UN13A | 995 | ACLNSTYEYIFNNC | 1005 | 7420 |
| | 420 | | | HELYSR | | |
| 948 | CAN3_366_650 | CAN3 | 989 | NPWGQVEWNGSWS | 366 | 6503 |
| | 3 | | | DR | | |
| 949 | AACT_106_760 | AACT | 963 | FNLTETSEAEIHQSF | 106 | 7604 |
| | 4 | | | QHLLR | | |
| 950 | A1AT_107_541 | A1AT | 956 | ADTHDEILEGLNFN | 107 | 5411 |
| | 1 | | | LTEIPEAQIHEGFQE | | |
| | | | | LLR | | |
| 951 | AGP1_33_5402 | AGP1 | 975 | QIPLCANLVPVPITN | 33 | 5402 |
| | | | | ATLDQITGK | | |
| 952 | FETUA_176_76 | FETUA | 976 | AALAAFNAQNNGS | 176 | 7600 |
| | 00 | | | NFQLEEISR | | |
| 953 | ITIH4_517_542 | ITIH5 | 994 | LPTQNITFQTESSVA | 517 | 5420.54 |
| | 0.5401 | | | EQEAEFQSPK | | 01 |
| 954 | PON1_324_650 | PON1 | 990 | VTQVYAENGTVLQ | 324 | 6502 |
| | 2 | | | GSTVASVYK | | |
| 955 | AGP1_33_6502 | AGP1 | 975 | QIPLCANL VPVPITN | 33 | 6502 |
| | | | | ATLDQITGK | | |

TABLE 71
Glycoproteins

| SEQ | Protein | | Uniprot |
|---|---|---|---|
| ID NO: | Abbreviation | Protein Name | ID |
| 956 | A1AT | Alpha-1-antitrypsin | P01009 |
| 957 | A2MG | Alpha-2-macroglobulin | P01023 |
| 958 | KLKB1 | Plasma Kallikrein | P03952 |
| 959 | THRB | Prothrombin | P00734 |
| 960 | CERU | Ceruloplasmin | P00450 |
| 961 | THRB | Prothrombin | P00734 |
| 962 | HPT | Haptoglobin | P00738 |
| 963 | AACT | Alpha-1-antichymotrypsin | P01011 |
| 964 | ANGT | Angiotensinogen | P01019 |
| 965 | A2MG | Alpha-2-macroglobulin | P01023 |
| 966 | CO5 | ComplementC5 | P01031 |
| 967 | KNG1 | Kininogen-1 | P01042 |
| 968 | IGJ | Immunoglobulin J chain | P01591 |
| 969 | IGG1 | Immunoglobulin heavy | P01857 |
| | | constant gamma 1 | |
| 970 | IGG4 | Immunoglobulin heavy | P01861 |
| | | constant gamma 4 | |
| 971 | IGM | Immunoglobulin heavy | P01871 |
| | | constant mu | |
| 972 | IGA2 | Immunoglobulin heavy | P01877 |
| | | constant alpha 2 | |
| 973 | APOC3 | Apolipoprotein C-III | P02656 |
| 974 | A2GL | Leucine-richAlpha-2- | P02750 |
| | | glycoprotein | |
| 975 | AGP1 | Alpha-1-acid glycoprotein 1 | P02763 |
| 976 | FETUA | Alpha-2-HS-glycoprotein | P02765 |
| 977 | TRFE | Serotransferrin | P02787 |
| 978 | HEMO | Hemopexin | P02790 |
| 979 | KLKB1 | Plasma Kallikrein | P03952 |
| 980 | VTNC | Vitronectin | P04004 |
| 981 | HRG | Histidine-rich Glycoprotein | P04196 |
| 982 | APOD | Apolipoprotein D | P05090 |
| 983 | IC1 | Plasma protease C1 inhibitor | P05155 |
| 984 | THBG | Thyroxine-bindingGlobulin | P05543 |
| 985 | CO2 | ComplementC2 | P06681 |
| 986 | CO8B | ComplementComponentC8BChain | P07358 |
| 987 | CFAH | ComplementFactorH | P08603 |
| 988 | CLUS | Clusterin | P10909 |
| 989 | CAN3 | Calpain-3 | P20807 |
| 990 | PON1 | Serum | P27169 |
| | | paraoxonase/arylesterase 1 | |
| 991 | AFAM | Afamin | P43652 |
| 992 | B2M | Beta-2-microglobulin | P61769 |
| 993 | FHR1 | Complement factor H-related | Q03591 |
| | | protein 1 | |
| 994 | ITIH4 | Inter-alpha-trypsin inhibitor | Q14624 |
| | | heavy chain H4 | |
| 995 | UN13A | Protein unc-13HomologA | Q9UPW8 |
| 996 | AGP2 | Alpha-1-acid glycoprotein 2 | P19652 |

TABLE 72
Protein abbreviation, glycosylation site, glycan
structure, precursor ion m/z, and product ion
m/z for transitions associated with melanoma

| Transition | | | | Precursor | Product |
|---|---|---|---|---|---|
| Number | Protein | Site | Structure | m/z | m/z |
| 673 | A2GL | N/A | N/A | 590.3 | 725.4 |
| 674 | ANGT | N/A | N/A | 719.4 | 316.2 |
| 675 | A1AT | 70 | 5412 | 1107.7 | 366.1 |
| 676 | HPT | 184 | 5412 | 1258.7 | 366.1 |
| 677 | HPT | 241 | 6513 | 1201.5 | 366.1 |
| 678 | HEMO | 187 | 5412 | 1253.2 | 366.1 |
| 679 | IC1 | 48 | 1102 | 883.4 | 274.1 |
| 680 | HPT | 184 | 6513 | 1138.4 | 366.1 |
| 681 | APOC3 | 74 | NONGLYCO- | 1069.2 | 1097.59 |
| | | | SYLATED | | |
| 682 | IGM | 209 | 5500 | 1042.4 | 366.1 |
| 683 | FETUA | 156 | 5412 | 1031.9 | 204.1 |
| 684 | B2M | N/A | N/A | 561.8 | 244.2 |
| 685 | IC1 | 253 | 5412 | 1114.2 | 204.1 |
| 686 | CERU | 138 | 5412 | 1062.2 | 366.1 |
| 687 | IGM | 209 | 5501 | 1115 | 366.1 |
| 688 | THRB | 416 | 5402 | 1076.5 | 274.1 |
| 689 | TRFE | 630 | 5412 | 1217.7 | 366.1 |
| 690 | FETUA | 176 | 6501 | 1161.7 | 366.1 |
| 691 | CO5 | 741 | 5412 | 1007.7 | 366.1 |
| 692 | FETUA | 176 | 5412 | 1180.2 | 366.1 |
| 693 | CFAH | 911 | 5401 | 1159.4 | 366.1 |
| 694 | IGG1 | 297 | 4511 | 1097.8 | 204.1 |
| 695 | A2MG | 247 | 5200 | 1239.1 | 1314.2 |
| 696 | CERU | 138 | 5402 | 1025.7 | 274.1 |
| 697 | IGA2 | 205 | 4510 | 923.5 | 366.1 |
| 698 | HRG | 125 | 5402 | 1056.2 | 366.1 |
| 699 | HPT | 207 | 121005 | 1378.9 | 366.1 |
| 700 | AACT | 106 | 7604 | 1184.9 | 274.1 |
| 701 | CERU | 397 | 6503 | 998.8 | 204.1 |
| 702 | HPT | 207 | 11904 | 1247.7 | 366.1 |

TABLE 73
Retention time, Δ retention time, and collision
energy for transitions associated with melanoma

| | Retention | Delta | |
|---|---|---|---|
| Transition | Time | Retention | Collision |
| Number | (min) | Time | Energy |
| 673 | 30.53 | 1.4 | 15 |
| 674 | 30.46 | 1.2 | 21 |
| 675 | 47.02 | 2 | 27 |
| 676 | 33.49 | 1.4 | 31 |
| 677 | 31.15 | 1.4 | 30 |
| 678 | 21.54 | 1.5 | 30 |
| 679 | 11.64 | 1.4 | 25 |
| 680 | 34.56 | 1.4 | 28 |
| 681 | 38.45 | N/A | N/A |
| 682 | 23.6 | 1.4 | 30 |
| 683 | 27.38 | 1.6 | 30 |
| 684 | 9.46 | 1.2 | 25 |
| 685 | 35.71 | 1.4 | 30 |
| 686 | 16.6 | 1.4 | 25 |
| 687 | 25.38 | 1.4 | 20 |
| 688 | 40.57 | 1.4 | 20 |
| 689 | 32.42 | 1.8 | 30 |
| 690 | 30.11 | 1.4 | 29 |
| 691 | 4.17 | 1.6 | 30 |
| 692 | 30.61 | 1.4 | 29 |
| 693 | 12.23 | 1.4 | 35 |
| 694 | 8.61 | 1.3 | 15 |
| 695 | 38.71 | 1.3 | 25 |
| 696 | 16.83 | 1.4 | 20 |
| 697 | 12.44 | 1.4 | 22 |
| 698 | 28.65 | 1.4 | 25 |
| 699 | 13.48 | 1.5 | 35 |
| 700 | 38.45 | 1.2 | 30 |
| 701 | 27.87 | 1.4 | 40 |
| 702 | 13.45 | 1.5 | 31 |

TABLE 74
Protein abbreviation, glycosylation site, glycan structure, precursor
ion m/z, and product ion m/z for transitions associated with NSCLC

| Transition | | | | Precursor | Product |
|---|---|---|---|---|---|
| Number | Protein | Site | Structure | m/z | m/z |
| 731 | TRFE | 630 | 6513 | 1105.6 | 366.1 |
| 732 | AGP1 | 93 | 6503 | 1195.3 | 366.1 |
| 733 | IGG2 | 297 | 5510 | 1043.8 | 366.1 |
| 734 | IGG1 | 297 | 5410 | 987.1 | 366.1 |
| 735 | AACT | 271 | 6502 | 1441.6 | 366.1 |
| 736 | AGP1 | 103 | 6503 | 1213.3 | 366.1 |
| 737 | IGG1 | 297 | 3410 | 879 | 204.1 |
| 738 | IGG1 | 297 | 5510 | 1054.7 | 366.1 |
| 739 | VTNC | 86 | 6503 | 1311.8 | 366.1 |
| 740 | HPT | 241 | 6513 | 1201.5 | 366.1 |
| 741 | CERU | 762 | 6523 | 1295 | 274.1 |
| 742 | HRG | 345 | 5412 | 994.4 | 366.1 |
| 743 | HPT | 207 | 5401 | 1124.8 | 366.1 |
| 744 | AGP1 | 93 | 8704 | 967.4 | 366.1 |
| 745 | HRG | 125 | 5402 | 1056.2 | 366.1 |
| 746 | A1AT | 271 | 5401 | 1224.5 | 366.1 |
| 747 | KNG1 | 205 | 5412 | 942.4 | 274.1 |
| 748 | TRFE | 432 | 5401 | 1131.1 | 366.1 |
| 749 | IGG2 | 297 | 5410 | 976.1 | 366.1 |
| 750 | TRFE | 630 | 5400 | 1035.6 | 366.1 |
| 751 | AGP1 | 93 | 7603 | 1286.6 | 366.1 |
| 752 | CERU | 762 | 6512 | 1186 | 366.1 |
| 753 | A1AT | 107 | 6502 | 1253.6 | 366.1 |
| 754 | KLKB1 | 494 | 5400 | 968.2 | 366.1 |
| 755 | IGG1 | 297 | 5411 | 1084.1 | 366.1 |
| 756 | HPT | 207 | 121005 | 1378.9 | 366.1 |
| 757 | FETUA | 176 | 5412 | 1180.2 | 366.1 |
| 758 | HPT | 241 | 5412 | 1383 | 366.1 |
| 759 | CFAH | 882 | 5401 | 984.7 | 366.1 |
| 760 | AGP1 | 93 | 6502 | 1122.5 | 366.1 |
| 761 | IC1 | 352 | 5412 | 1167.3 | 366.1 |
| 762 | HEMO | 187 | NONGLYCO- | 703.3 | 566.3 |
| | | | SYLATED | | |
| 763 | KLKB1 | 396 | 5401 | 1069.2 | 204.1 |
| 764 | IGJ | 71 | 5412 | 1193.8 | 366.1 |
| 765 | AGP12 | 72 | 7614 | 1313.1 | 366.1 |
| 766 | TRFE | 630 | 5401 | 1108.4 | 366.1 |
| 767 | TRFE | 630 | 5411 | 1144.9 | 366.1 |
| 768 | IGM | 209 | 5512 | 1224.1 | 366.1 |
| 769 | KNG1 | 137 | NONGLYCO- | 1190.6 | 1349.7 |
| | | | SYLATED | | |
| 770 | FHR1 | 126 | 5402 | 1265.5 | 366.1 |
| 771 | IGG1 | 297 | 4500 | 951.7 | 204.1 |
| 772 | AGP1 | 93 | 7612 | 1250.3 | 366.1 |
| 773 | A1AT | 271 | 5402 | 991.2 | 366.1 |
| 774 | A1AT | 271 | 6503 | 1155.5 | 274.1 |
| 775 | KNG1 | 294 | 5412 | 946.9 | 204.1 |
| 776 | CO2 | 621 | 6200 | 945.1 | 829.4 |
| 777 | HRG | 271 | 2202 | 710.8 | 274.1 |
| 778 | APOD | 98 | 5412 | 1152.5 | 274.1 |
| 779 | AFAM | 33 | 5402 | 851.1 | 366.1 |

TABLE 75
Retention time, Δ retention time, and collision
energy for transitions associated with NSCLC

| | Retention | Delta | |
|---|---|---|---|
| Transition | Time | Retention | Collision |
| Number | (min) | Time | Energy |
| 731 | 33.44 | 1.4 | 27 |
| 732 | 23.77 | 1.4 | 25 |
| 733 | 12.99 | 1.2 | 25 |
| 734 | 7.9 | 1.3 | 24 |
| 735 | 30.91 | 1.4 | 35 |
| 736 | 5.97 | 1.6 | 30 |
| 737 | 8.01 | 1.3 | 21 |
| 738 | 8.09 | 1.3 | 20 |
| 739 | 19.44 | 1.4 | 37 |
| 740 | 31.27 | 1.4 | 30 |
| 741 | 20.77 | 1.4 | 25 |
| 742 | 6.55 | 1.4 | 25 |
| 743 | 14.38 | 1.5 | 30 |
| 744 | 23.81 | 1.4 | 23 |
| 745 | 28.7 | 1.4 | 25 |
| 746 | 37.62 | 1.4 | 30 |
| 747 | 16.93 | 1.4 | 20 |
| 748 | 26.42 | 1.4 | 28 |
| 749 | 12.87 | 1.2 | 20 |
| 750 | 30.62 | 1.4 | 25 |
| 751 | 23.61 | 1.4 | 25 |
| 752 | 19.97 | 1.4 | 36 |
| 753 | 42.79 | 1.6 | 30 |
| 754 | 30.4 | 1.4 | 30 |
| 755 | 8.35 | 1.3 | 27 |
| 756 | 13.41 | 1.5 | 35 |
| 757 | 30.64 | 1.4 | 29 |
| 758 | 30.52 | 1.4 | 35 |
| 759 | 14.9 | 1.6 | 25 |
| 760 | 23.17 | 1.4 | 28 |
| 761 | 39.45 | 1.5 | 30 |
| 762 | 21.83 | 1.4 | 20 |
| 763 | 39.8 | 1.4 | 25 |
| 764 | 16.04 | 1.4 | 25 |
| 765 | 41.23 | 1.4 | 27 |
| 766 | 31.54 | 1.8 | 27 |
| 767 | 30.97 | 1.6 | 30 |
| 768 | 26.21 | 1.4 | 30 |
| 769 | 38.59 | 1 | 30 |
| 770 | 11.55 | 1.5 | 30 |
| 771 | 8.13 | 1.3 | 23 |
| 772 | 22.84 | 1.4 | 31 |
| 773 | 38.43 | 1.4 | 24 |
| 774 | 38.74 | 1.4 | 30 |
| 775 | 22.96 | 1.4 | 20 |
| 776 | 16.26 | 1.4 | 25 |
| 777 | 6.74 | 1.4 | 15 |
| 778 | 24.42 | 1.4 | 30 |
| 779 | 11.62 | 1.2 | 20 |

TABLE 76
Glycan Structures

| Glycan | | |
|---|---|---|
| Structure | | |
| GL NO. | Structure | Composition |
| 1102 | [symbol] | Hex(1)HexNAc(1)Fuc(0)NeuAc(2) |
| 2202 | [symbol] | Hex(2)HexNAc(2)Fuc(0)NeuAc(2) |
| 3410 | [symbol] | Hex(3)HexNAc(4)Fuc(1)NeuAc(0) |
| 3500 | [symbol] | Hex(3)HexNAc(5)Fuc(0)NeuAc(0) |
| 4400 | [symbol] | Hex(4)HexNAc(4)Fuc(0)NeuAc(0) |
| 4401 | [symbol] | Hex(4)HexNAc(4)Fuc(0)NeuAc(1) |
| 4410 | [symbol] | Hex(4)HexNAc(4)Fuc(1)NeuAc(0) |
| 4411 | [symbol] | Hex(4)HexNAc(4)Fuc(1)NeuAc(1) |
| 4500 | [symbol] | Hex(4)HexNAc(5)Fuc(0)NeuAc(0) |
| 4510 | [symbol] | Hex(4)HexNAc(5)Fuc(1)NeuAc(0) |
| 4511 | [symbol] | Hex(4)HexNAc(5)Fuc(1)NeuAc(1) |
| 5200 | [symbol] | Hex(5)HexNAc(2)Fuc(0)NeuAc(0) |
| 5400 | [symbol] | Hex(5)HexNAc(4)Fuc(0)NeuAc(0) |
| 5401 | [symbol] | Hex(5)HexNAc(4)Fuc(0)NeuAc(1) |
| 5402 | [symbol] | Hex(5)HexNAc(4)Fuc(0)NeuAc(2) |
| 5410 | [symbol] | Hex(5)HexNAc(4)Fuc(1)NeuAc(0) |
| 5411 | [symbol] | Hex(5)HexNAc(4)Fuc(1)NeuAc(1) |
| 5412 | [symbol] | Hex(5)HexNAc(4)Fuc(1)NeuAc(2) |
| 5420 | [symbol] | Hex(5)HexNAc(4)Fuc(2)NeuAc(0) |
| 5421 | [symbol] | Hex(5)HexNAc(4)Fuc(2)NeuAc(1) |
| 5431 | [symbol] | Hex(5)HexNAc(4)Fuc(3)NeuAc(1) |
| 5500 | [symbol] | Hex(5)HexNAc(5)Fuc(0)NeuAc(0) |
| 5501 | [symbol] | Hex(5)HexNAc(5)Fuc(0)NeuAc(1) |
| 5502 | [symbol] | Hex(5)HexNAc(5)Fuc(0)NeuAc(2) |
| 5510 | [symbol] | Hex(5)HexNAc(5)Fuc(1)NeuAc(0) |
| 5511 | [symbol] | Hex(5)HexNAc(5)Fuc(1)NeuAc(1) |
| 5512 | [symbol] | Hex(5)HexNAc(5)Fuc(1)NeuAc(2) |
| 6200 | [symbol] | Hex(6)HexNAc(2)Fuc(0)NeuAc(0) |
| 6501 | [symbol] | Hex(6)HexNAc(5)Fuc(0)NeuAc(1) |
| 6502 | [symbol] | Hex(6)HexNAc(5)Fuc(0)NeuAc(2) |
| 6503 | [symbol] | Hex(6)HexNAc(5)Fuc(0)NeuAc(3) |
| 6512 | [symbol] | Hex(6)HexNAc(5)Fuc(1)NeuAc(2) |
| 6513 | [symbol] | Hex(6)HexNAc(5)Fuc(1)NeuAc(3) |
| 6520 | [symbol] | Hex(6)HexNAc(5)Fuc(2)NeuAc(0) |
| 6523 | [symbol] | Hex(6)HexNAc(5)Fuc(2)NeuAc(3) |
| 6610 | [symbol] | Hex(6)HexNAc(6)Fuc(1)NeuAc(0) |
| 7420 | [symbol] | Hex(7)HexNAc(4)Fuc(2)NeuAc(0) |
| 7512 | [symbol] | Hex(7)HexNAc(5)Fuc(1)NeuAc(2) |
| 7600 | [symbol] | Hex(7)HexNAc(6)Fuc(0)NeuAc(0) |
| 7602 | [symbol] | Hex(7)HexNAc(6)Fuc(0)NeuAc(2) |
| 7603 | [symbol] | Hex(7)HexNAc(6)Fuc(0)NeuAc(3) |
| 7604 | [symbol] | Hex(7)HexNAc(6)Fuc(0)NeuAc(4) |
| 7612 | [symbol] | Hex(7)HexNAc(6)Fuc(1)NeuAc(2) |
| 7614 | [symbol] | Hex(7)HexNAc(6)Fuc(1)NeuAc(4) |
| 8704 | [symbol] | Hex(8)HexNAc(7)Fuc(0)NeuAc(4) |
| 10803 (5401 & 5402) (two glycans on the same peptide) | [symbol] | 5401: Hex(5)HexNAc(4)Fuc(0)NeuAc(1) and 5402: Hex(5)HexNAc(4)Fuc(0)NeuAc(2) |
| 11904 (5402 and 6502) | [symbol] | 5402: Hex(5)HexNAc(4)Fuc(0)NeuAc(2) 6502: Hex(6)HexNAc(5)Fuc(0)NeuAc(2) |
| 121005 (6502) | [symbol] | 6502: Hex(6)HexNAc(5)Fuc(0)NeuAc(2) |
| 121005 (6503) | [symbol] | 6503: Hex(6)HexNAc(5)Fuc(0)NeuAc(3) |

Legend for Table 76
▪ Glc [symbol]
Gal [symbol]
Man [symbol]
Fuc [symbol]
Neu5Ac
● GlcNAc [symbol]
GalNAc [symbol]
ManNAc

In some embodiments, provided herein are methods for diagnosing a melanoma condition (metastatic melanoma) comprising detecting one or more biomarkers. In some embodiments, the one or more biomarkers comprise one or more glycopeptides. In some embodiments, the one or more biomarkers comprises one or more peptide structures set forth in Table 61. In some embodiments, the method comprises detecting one or more glycopeptides comprising a sequence set forth in SEQ ID NOS: 570-595. In some embodiments, the method comprises detecting one or more glycopeptides comprising a sequence set forth in SEQ ID NOS: 570-595. In some embodiments, the method comprises detecting one or more glycopeptides comprising a sequence set forth in SEQ ID NOS: 570-595. In some embodiments, the method comprises detecting one or more glycopeptides comprising a sequence set forth in SEQ ID NOS: 570-595. In some embodiments, the glycopeptide comprises a glycan with the structures in Table 61. In some embodiments, the glycopeptide is a glycopeptide of a glycoprotein provided in Table 62A. In some embodiments, the glycopeptide is a glycopeptide provided in Table 70. In some embodiments the glycopeptide comprises a sequence set forth in SEQ ID NO:826-955. In some embodiments, the glycopeptide is a glycopeptide a glycoprotein comprising SEQ ID NO:550-569.

In some embodiments, the diagnosis is based upon presence and/or amount of at least one, at least two, at least three, at least four, at least five, at least six, at least seven or eight peptide structures from Table 61. In some embodiments, the diagnosis is based upon the presence and/or amount of one or more peptides comprising the amino acid sequence of SEQ ID NOS: 570-595. In some embodiments, the diagnosis is based upon the presence and/or amount of two or more peptides comprising the amino acid sequence of SEQ ID NOS: 570-595. In some embodiments, the diagnosis is based upon the presence and/or amount of three or more peptides comprising the amino acid sequence of SEQ ID NOS: 570-595. In some embodiments, the diagnosis is based upon the presence and/or amount of four or more peptides comprising the amino acid sequence of SEQ ID NOS: 570-595. In some embodiments, the diagnosis is based upon the presence and/or amount of five or more peptides comprising the amino acid sequence of SEQ ID NOS: 570-595. In some embodiments, the diagnosis is based upon the presence and/or amount of six or more peptides comprising the amino acid sequence of SEQ ID NOS: 570-595. In some embodiments, the diagnosis is based upon the presence and/or amount of seven or more peptides comprising the amino acid sequence of SEQ ID NOS: 570-595. In some embodiments, the diagnosis is based upon the presence and/or amount of each of the peptides comprising the amino acid sequence of SEQ ID NOS: 570-595. In some embodiments, the glycopeptide is a glycopeptide of a glycoprotein provided in Table 62A. In some embodiments, the glycopeptide is a glycopeptide provided in Table 70. In some embodiments the glycopeptide comprises a sequence set forth in SEQ ID NO: 826-955. In some embodiments, the glycopeptide is a glycopeptide a glycoprotein comprising SEQ ID NO: 550-569.

In some embodiments, the diagnosis is based upon the presence and/or amount of one or more peptides consisting of the amino acid sequence of SEQ ID NOS: 570-595. In some embodiments, the diagnosis is based upon the presence and/or amount of two or more peptides consisting of the amino acid sequence of SEQ ID NOS: 570-595. In some embodiments, the diagnosis is based upon the presence and/or amount of three or more peptides consisting of the amino acid sequence of SEQ ID NOS: 570-595. In some embodiments, the diagnosis is based upon the presence and/or amount of four or more peptides consisting of the amino acid sequence of SEQ ID NOS: 570-595. In some embodiments, the diagnosis is based upon the presence and/or amount of five or more peptides consisting of the amino acid sequence of SEQ ID NOS: 570-595. In some embodiments, the diagnosis is based upon the presence and/or amount of six or more peptides consisting of the amino acid sequence of SEQ ID NOS: 570-595. In some embodiments, the diagnosis is based upon the presence and/or amount of seven or more peptides consisting of the amino acid sequence of SEQ ID NOS: 570-595. In some embodiments, the diagnosis is based upon the presence and/or amount of each of the peptides consisting of the amino acid sequence of SEQ ID NOS: 570-595. In some embodiments, the glycopeptide is a glycopeptide of a glycoprotein provided in Table 62A. In some embodiments, the glycopeptide is a glycopeptide provided in Table 70. In some embodiments the glycopeptide comprises a sequence set forth in SEQ ID NO: 826-955. In some embodiments, the glycopeptide is a glycopeptide a glycoprotein comprising SEQ ID NO: 550-569.

In some embodiments, the diagnosis is based upon the presence and/or amount of one or more peptides comprising the amino acid sequence of SEQ ID NOS: 570-595. In some embodiments, the diagnosis is based upon the presence and/or amount of two or more peptides comprising the amino acid sequence of SEQ ID NOS: 570-595. In some embodiments, the diagnosis is based upon the presence and/or amount of three or more peptides comprising the amino acid sequence of SEQ ID NOS: 570-595. In some embodiments, the diagnosis is based upon the presence and/or amount of four or more peptides comprising the amino acid sequence of SEQ ID NOS: 570-595. In some embodiments, the diagnosis is based upon the presence and/or amount of five or more peptides comprising the amino acid sequence of SEQ ID NOS: 570-595. In some embodiments, the diagnosis is based upon the presence and/or amount of six or more peptides comprising the amino acid sequence of SEQ ID NOS: 570-595. In some embodiments, the diagnosis is based upon the presence and/or amount of seven or more peptides comprising the amino acid sequence of SEQ ID NOS: 570-595. In some embodiments, the diagnosis is based upon the presence and/or amount of each of the peptides comprising the amino acid sequence of SEQ ID NOS: 570-595. In some embodiments, the glycopeptide is a glycopeptide of a glycoprotein provided in Table 62A. In some embodiments, the glycopeptide is a glycopeptide provided in Table 70. In some embodiments the glycopeptide comprises a sequence set forth in SEQ ID NO: 826-955. In some embodiments, the glycopeptide is a glycopeptide a glycoprotein comprising SEQ ID NO: 550-569.

In some embodiments, the diagnosis is based upon the presence and/or amount of one or more peptides consisting of the amino acid sequence of SEQ ID NOS: 570-595. In some embodiments, the diagnosis is based upon the presence and/or amount of two or more peptides consisting of the amino acid sequence of SEQ ID NOS: 570-595. In some embodiments, the diagnosis is based upon the presence and/or amount of three or more peptides consisting of the amino acid sequence of SEQ ID NOS: 570-595. In some embodiments, the diagnosis is based upon the presence and/or amount of four or more peptides consisting of the amino acid sequence of SEQ ID NOS: 570-595. In some embodiments, the diagnosis is based upon the presence and/or amount of five or more peptides consisting of the amino acid sequence of SEQ ID NOS: 570-595. In some embodiments, the diagnosis is based upon the presence and/or amount of six or more peptides consisting of the amino acid sequence of SEQ ID NOS: 570-595. In some embodiments, the diagnosis is based upon the presence and/or amount of seven or more peptides consisting of the amino acid sequence of SEQ ID NOS: 570-595. In some embodiments, the diagnosis is based upon the presence and/or amount of each of the peptides consisting of the amino acid sequence of SEQ ID NOS: 570-595. In some embodiments, the glycopeptide is a glycopeptide of a glycoprotein provided in Table 62A. In some embodiments, the glycopeptide is a glycopeptide provided in Table 70. In some embodiments the glycopeptide comprises a sequence set forth in SEQ ID NO: 826-955. In some embodiments, the glycopeptide is a glycopeptide a glycoprotein comprising SEQ ID NO: 550-569.

In some embodiments, provided herein is a method of treating a melanoma condition (metastatic melanoma) in an individual based upon the presence, absence, or amount of one or more peptide structures set forth in Table 61. In some embodiments, one or more peptide structures set forth in SEQ ID NOS: 570-595 is detected. In some embodiments, the method further comprises delivering a therapeutic agent based upon the presence, absence, or amount of one or more peptide structures set forth in Table 61. In some embodiments, the method comprises selecting a therapeutic agent based upon the presence, absence, or amount of one or more peptide structures set forth in Table 61. In some embodiments, the therapeutic agent is a chemotherapeutic agent and/or a hormone therapy.

In some embodiments, provided herein are methods for diagnosing a melanoma condition (metastatic melanoma) comprising detecting one or more biomarkers. In some embodiments, the one or more biomarkers comprise one or more glycopeptides. In some embodiments, the one or more biomarkers comprises one or more peptide structures set forth in Table 66. In some embodiments, the method comprises detecting one or more glycopeptides comprising a sequence set forth in SEQ ID NOs: 673-703. In some embodiments, the method comprises detecting one or more glycopeptides comprising a sequence set forth in SEQ ID NOs: 673-703. In some embodiments, the method comprises detecting one or more glycopeptides comprising a sequence set forth in SEQ ID NOs: 673-703. In some embodiments, the method comprises detecting one or more glycopeptides comprising a sequence set forth in SEQ ID NOs: 673-703. In some embodiments, the glycopeptide comprises a glycan with the structures in Table 66. In some embodiments the glycopeptide is a glycopeptide of a glycoprotein provided in Table 67. In some embodiments, the glycopeptide is a glycopeptide of a glycoprotein provided in SEQ ID NO: 704-730.

In some embodiments, the diagnosis is based upon presence and/or amount of at least one, at least two, at least three, at least four, at least five, at least six, at least seven or eight peptide structures from Table 66. In some embodiments, the diagnosis is based upon the presence and/or amount of one or more peptides comprising the amino acid sequence of SEQ ID NOs: 673-708. In some embodiments, the diagnosis is based upon the presence and/or amount of two or more peptides comprising the amino acid sequence of SEQ ID NOs: 673-708. In some embodiments, the diagnosis is based upon the presence and/or amount of three or more peptides comprising the amino acid sequence of SEQ ID NOs: 673-708. In some embodiments, the diagnosis is based upon the presence and/or amount of four or more peptides comprising the amino acid sequence of SEQ ID NOs: 673-708. In some embodiments, the diagnosis is based upon the presence and/or amount of five or more peptides comprising the amino acid sequence of SEQ ID NOs: 673-708. In some embodiments, the diagnosis is based upon the presence and/or amount of six or more peptides comprising the amino acid sequence of SEQ ID NOs: 673-708. In some embodiments, the diagnosis is based upon the presence and/or amount of seven or more peptides comprising the amino acid sequence of SEQ ID NOs: 673-708. In some embodiments, the diagnosis is based upon the presence and/or amount of each of the peptides comprising the amino acid sequence of SEQ ID NOs: 673-708. In some embodiments the glycopeptide is a glycopeptide of a glycoprotein provided in Table 67. In some embodiments, the glycopeptide is a glycopeptide of a glycoprotein provided in SEQ ID NO: 704-730.

In some embodiments, the diagnosis is based upon the presence and/or amount of one or more peptides consisting of the amino acid sequence of SEQ ID NOs: 673-708. In some embodiments, the diagnosis is based upon the presence and/or amount of two or more peptides consisting of the amino acid sequence of SEQ ID NOs: 673-708. In some embodiments, the diagnosis is based upon the presence and/or amount of three or more peptides consisting of the amino acid sequence of SEQ ID NOs: 673-708. In some embodiments, the diagnosis is based upon the presence and/or amount of four or more peptides consisting of the amino acid sequence of SEQ ID NOs: 673-708. In some embodiments, the diagnosis is based upon the presence and/or amount of five or more peptides consisting of the amino acid sequence of SEQ ID NOs: 673-708. In some embodiments, the diagnosis is based upon the presence and/or amount of six or more peptides consisting of the amino acid sequence of SEQ ID NOs: 673-708. In some embodiments, the diagnosis is based upon the presence and/or amount of seven or more peptides consisting of the amino acid sequence of SEQ ID NOs: 673-708. In some embodiments, the diagnosis is based upon the presence and/or amount of each of the peptides consisting of the amino acid sequence of SEQ ID NOs: 673-708. In some embodiments the glycopeptide is a glycopeptide of a glycoprotein provided in Table 67. In some embodiments, the glycopeptide is a glycopeptide of a glycoprotein provided in SEQ ID NO: 704-730.

In some embodiments, the diagnosis is based upon the presence and/or amount of one or more peptides comprising the amino acid sequence of SEQ ID NOs: 673-708. In some embodiments, the diagnosis is based upon the presence and/or amount of two or more peptides comprising the amino acid sequence of SEQ ID NOs: 673-708. In some embodiments, the diagnosis is based upon the presence and/or amount of three or more peptides comprising the amino acid sequence of SEQ ID NOs: 673-708. In some embodiments, the diagnosis is based upon the presence and/or amount of four or more peptides comprising the amino acid sequence of SEQ ID NOs: 673-708. In some embodiments, the diagnosis is based upon the presence and/or amount of five or more peptides comprising the amino acid sequence of SEQ ID NOs: 673-708. In some embodiments, the diagnosis is based upon the presence and/or amount of six or more peptides comprising the amino acid sequence of SEQ ID NOs: 673-708. In some embodiments, the diagnosis is based upon the presence and/or amount of seven or more peptides comprising the amino acid sequence of SEQ ID NOs: 673-708. In some embodiments, the diagnosis is based upon the presence and/or amount of each of the peptides comprising the amino acid sequence of SEQ ID NOs: 673-708. In some embodiments the glycopeptide is a glycopeptide of a glycoprotein provided in Table 67. In some embodiments, the glycopeptide is a glycopeptide of a glycoprotein provided in SEQ ID NO: 704-730.

In some embodiments, the diagnosis is based upon the presence and/or amount of one or more peptides consisting of the amino acid sequence of SEQ ID NOs: 673-708. In some embodiments, the diagnosis is based upon the presence and/or amount of two or more peptides consisting of the amino acid sequence of SEQ ID NOs: 673-708. In some embodiments, the diagnosis is based upon the presence and/or amount of three or more peptides consisting of the amino acid sequence of SEQ ID NOs: 673-708. In some embodiments, the diagnosis is based upon the presence and/or amount of four or more peptides consisting of the amino acid sequence of SEQ ID NOs: 673-708. In some embodiments, the diagnosis is based upon the presence and/or amount of five or more peptides consisting of the amino acid sequence of SEQ ID NOs: 673-708. In some embodiments, the diagnosis is based upon the presence and/or amount of six or more peptides consisting of the amino acid sequence of SEQ ID NOs: 673-708. In some embodiments, the diagnosis is based upon the presence and/or amount of seven or more peptides consisting of the amino acid sequence of SEQ ID NOs: 673-708. In some embodiments, the diagnosis is based upon the presence and/or amount of each of the peptides consisting of the amino acid sequence of SEQ ID NOs: 673-708. In some embodiments the glycopeptide is a glycopeptide of a glycoprotein provided in Table 67. In some embodiments, the glycopeptide is a glycopeptide of a glycoprotein provided in SEQ ID NO: 704-730.

In some embodiments, provided herein is a method of treating a melanoma condition (metastatic melanoma) in an individual based upon the presence, absence, or amount of one or more peptide structures set forth in Table 66. In some embodiments, one or more peptide structures set forth in SEQ ID NOs: 673-708 is detected. In some embodiments, the method further comprises delivering a therapeutic agent based upon the presence, absence, or amount of one or more peptide structures set forth in Table 66. In some embodiments, the method comprises selecting a therapeutic agent based upon the presence, absence, or amount of one or more peptide structures set forth in Table 66. In some embodiments, the therapeutic agent is a chemotherapeutic agent and/or a hormone therapy. In some embodiments the glycopeptide is a glycopeptide of a glycoprotein provided in Table 67. In some embodiments, the glycopeptide is a glycopeptide of a glycoprotein provided in SEQ ID NO: 704-730.

In some embodiments, provided herein are methods for diagnosing non-small-cell lung cancer (NSCLC) comprising detecting one or more biomarkers. In some embodiments, the one or more biomarkers comprise one or more glycopeptides. In some embodiments, the one or more biomarkers comprises one or more peptide structures set forth in Table 68. In some embodiments, the method comprises detecting one or more glycopeptides comprising a sequence set forth in SEQ ID NOs: 731-779. In some embodiments, the method comprises detecting one or more glycopeptides comprising a sequence set forth in SEQ ID NOs: 731-779. In some embodiments, the method comprises detecting one or more glycopeptides comprising a sequence set forth in SEQ ID NOs: 731-779. In some embodiments, the method comprises detecting one or more glycopeptides comprising a sequence set forth in SEQ ID NOs: 731-779. In some embodiments, the glycopeptide comprises a glycan with the structures in Table 68. In some embodiments, the glycopeptide is a glycopeptide of a glycoprotein provided in Table 69. In some embodiments, the glycopeptide is a glycopeptide of a glycoprotein comprising a sequence set forth in SEQ ID NO: 780-825.

In some embodiments, the diagnosis is based upon presence and/or amount of at least one, at least two, at least three, at least four, at least five, at least six, at least seven or eight peptide structures from Table 68. In some embodiments, the diagnosis is based upon the presence and/or amount of one or more peptides comprising the amino acid sequence of SEQ ID NOs: 731-779. In some embodiments, the diagnosis is based upon the presence and/or amount of two or more peptides comprising the amino acid sequence of SEQ ID NOs: 731-779. In some embodiments, the diagnosis is based upon the presence and/or amount of three or more peptides comprising the amino acid sequence of SEQ ID NOs: 731-779. In some embodiments, the diagnosis is based upon the presence and/or amount of four or more peptides comprising the amino acid sequence of SEQ ID NOs: 731-779. In some embodiments, the diagnosis is based upon the presence and/or amount of five or more peptides comprising the amino acid sequence of SEQ ID NOs: 731-779. In some embodiments, the diagnosis is based upon the presence and/or amount of six or more peptides comprising the amino acid sequence of SEQ ID NOs: 731-779. In some embodiments, the diagnosis is based upon the presence and/or amount of seven or more peptides comprising the amino acid sequence of SEQ ID NOs: 731-779. In some embodiments, the diagnosis is based upon the presence and/or amount of each of the peptides comprising the amino acid sequence of SEQ ID NOs: 731-779. In some embodiments, the glycopeptide is a glycopeptide of a glycoprotein provided in Table 69. In some embodiments, the glycopeptide is a glycopeptide of a glycoprotein comprising a sequence set forth in SEQ ID NO: 780-825.

In some embodiments, the diagnosis is based upon the presence and/or amount of one or more peptides consisting of the amino acid sequence of SEQ ID NOs: 731-779. In some embodiments, the diagnosis is based upon the presence and/or amount of two or more peptides consisting of the amino acid sequence of SEQ ID NOs: 731-779. In some embodiments, the diagnosis is based upon the presence and/or amount of three or more peptides consisting of the amino acid sequence of SEQ ID NOs: 731-779. In some embodiments, the diagnosis is based upon the presence and/or amount of four or more peptides consisting of the amino acid sequence of SEQ ID NOS: 570-595. In some embodiments, the diagnosis is based upon the presence and/or amount of five or more peptides consisting of the amino acid sequence of SEQ ID NOs: 731-779. In some embodiments, the diagnosis is based upon the presence and/or amount of six or more peptides consisting of the amino acid sequence of SEQ ID NOs: 731-779. In some embodiments, the diagnosis is based upon the presence and/or amount of seven or more peptides consisting of the amino acid sequence of SEQ ID NOs: 731-779. In some embodiments, the diagnosis is based upon the presence and/or amount of each of the peptides consisting of the amino acid sequence of SEQ ID NOs: 731-779. In some embodiments, the glycopeptide is a glycopeptide of a glycoprotein provided in Table 69. In some embodiments, the glycopeptide is a glycopeptide of a glycoprotein comprising a sequence set forth in SEQ ID NO: 780-825.

In some embodiments, the diagnosis is based upon the presence and/or amount of one or more peptides comprising the amino acid sequence of SEQ ID NOs: 731-779. In some embodiments, the diagnosis is based upon the presence and/or amount of two or more peptides comprising the amino acid sequence of SEQ ID NOs: 731-779. In some embodiments, the diagnosis is based upon the presence and/or amount of three or more peptides comprising the amino acid sequence of SEQ ID NOs: 731-779. In some embodiments, the diagnosis is based upon the presence and/or amount of four or more peptides comprising the amino acid sequence of SEQ ID NOs: 731-779. In some embodiments, the diagnosis is based upon the presence and/or amount of five or more peptides comprising the amino acid sequence of SEQ ID NOs: 731-779. In some embodiments, the diagnosis is based upon the presence and/or amount of six or more peptides comprising the amino acid sequence of SEQ ID NOs: 731-779. In some embodiments, the diagnosis is based upon the presence and/or amount of seven or more peptides comprising the amino acid sequence of SEQ ID NOs: 731-779. In some embodiments, the diagnosis is based upon the presence and/or amount of each of the peptides comprising the amino acid sequence of SEQ ID NOs: 731-779. In some embodiments, the glycopeptide is a glycopeptide of a glycoprotein provided in Table 69. In some embodiments, the glycopeptide is a glycopeptide of a glycoprotein comprising a sequence set forth in SEQ ID NO: 780-825.

In some embodiments, the diagnosis is based upon the presence and/or amount of one or more peptides consisting of the amino acid sequence of SEQ ID NOs: 731-779. In some embodiments, the diagnosis is based upon the presence and/or amount of two or more peptides consisting of the amino acid sequence of SEQ ID NOs: 731-779. In some embodiments, the diagnosis is based upon the presence and/or amount of three or more peptides consisting of the amino acid sequence of SEQ ID NOs: 731-779. In some embodiments, the diagnosis is based upon the presence and/or amount of four or more peptides consisting of the amino acid sequence of SEQ ID NOs: 731-779. In some embodiments, the diagnosis is based upon the presence and/or amount of five or more peptides consisting of the amino acid sequence of SEQ ID NOs: 731-779. In some embodiments, the diagnosis is based upon the presence and/or amount of six or more peptides consisting of the amino acid sequence of SEQ ID NOs: 731-779. In some embodiments, the diagnosis is based upon the presence and/or amount of seven or more peptides consisting of the amino acid sequence of SEQ ID NOs: 731-779. In some embodiments, the diagnosis is based upon the presence and/or amount of each of the peptides consisting of the amino acid sequence of SEQ ID NOs: 731-779. In some embodiments, the glycopeptide is a glycopeptide of a glycoprotein provided in Table 69. In some embodiments, the glycopeptide is a glycopeptide of a glycoprotein comprising a sequence set forth in SEQ ID NO: 780-825.

In some embodiments, provided herein is a method of treating non-small-cell lung cancer (NSCLC) in an individual based upon the presence, absence, or amount of one or more peptide structures set forth in Table 68. In some embodiments, one or more peptide structures set forth in SEQ ID NOs: 731-779 is detected. In some embodiments, the method further comprises delivering a therapeutic agent based upon the presence, absence, or amount of one or more peptide structures set forth in Table 68. In some embodiments, the method comprises selecting a therapeutic agent based upon the presence, absence, or amount of one or more peptide structures set forth in Table 68. In some embodiments, the therapeutic agent is a chemotherapeutic agent and/or a hormone therapy. In some embodiments, the glycopeptide is a glycopeptide of a glycoprotein provided in Table 69. In some embodiments, the glycopeptide is a glycopeptide of a glycoprotein comprising a sequence set forth in SEQ ID NO: 780-825.

In the descriptions herein, it is understood that every description, variation, embodiment or aspect of a biomarker, peptide, glycopeptide, glycoprotein may be combined with every description, variation, embodiment or aspect of other biomarkers, peptides, glycopeptide, glycoproteins the same as if each and every combination of descriptions is specifically and individually listed.

### II. Biomarkers for Determining Subject with NSCLC would Likely Benefit from Therapy

Provided herein are glycopeptide biomarkers. These biomarkers are useful for a variety of applications, including determining whether a subject is likely to benefit from pembrolizumab therapy. In some embodiments, the subject has NSCLC.. In some embodiments, the biomarkers are useful for classifying a patient so that the patient receives the appropriate medical treatment. In some embodiments, the biomarkers are useful for treating or ameliorating a disease or condition in patient by, for example, identifying a therapeutic agent with which to treat a patient.

### PROTEINS AND GLYCOPEPTIDES

TABLE 77
Glycoproteins associated with NSCLC

| SEQ ID | Protein | | Uniprot | |
|---|---|---|---|---|
| NO. | Abbreviation | Protein Name | ID | Protein Sequence |
| 997 | A1AT | Alpha-1- | P01009 | MPSSVSWGILLLAGLCC |
| | | antitrypsin | | LVPVSLAEDPQGDAAQ |
| | | | | KTDTSHHDQDHPTFNKI |
| | | | | TPNLAEFAFSLYRQLAH |
| | | | | QSNSTNIFFSPVSIATAF |
| | | | | AMLSLGTKADTHDEILE |
| | | | | GLNFNLTEIPEAQIHEGF |
| | | | | QELLRTLNQPDSQLQLT |
| | | | | TGNGLFLSEGLKLVDKF |
| | | | | LEDVKKLYHSEAFTVNF |
| | | | | GDTEEAKKQINDYVEK |
| | | | | GTQGKIVDLVKELDRDT |
| | | | | VFALVNYIFFKGKWERP |
| | | | | FEVKDTEEEDFHVDQVT |
| | | | | TVKVPMMKRLGMFNIQ |
| | | | | HCKKLSSWVLLMKYLG |
| | | | | NATAIFFLPDEGKLQHL |
| | | | | ENELTHDIITKFLENEDR |
| | | | | RSASLHLPKLSITGTYDL |
| | | | | KSVLGQLGITKVFSNGA |
| | | | | DLSGVTEEAPLKLSKAV |
| | | | | HKAVLTIDEKGTEAAG |
| | | | | AMFLEAIPMSIPPEVKFN |
| | | | | KPFVFLMIEQNTKSPLF |
| | | | | MGKVVNPTQK |
| 998 | AGP1 | Alpha-1-acid | P02763 | MALSWVLTVLSLLPLLE |
| | | glycoprotein 1 | | AQIPLCANLVPVPITNAT |
| | | | | LDQITGKWFYIASAFRN |
| | | | | EEYNKSVQEIQATFFYF |
| | | | | TPNKTEDTIFLREYQTR |
| | | | | QDQCIYNTTYLNVQRE |
| | | | | NGTISRYVGGQEHFAHL |
| | | | | LILRDTKTYMLAFDVND |
| | | | | EKNWGLSVYADKPETT |
| | | | | KEQLGEFYEALDCLRIP |
| | | | | KSDVVYTDWKKDKCEP |
| | | | | LEKQHEKERKQEEGES |
| 999 | AGP2 | Alpha-1-acid | P19652 | MALSWVLTVLSLLPLLE |
| | | glycoprotein 2 | | AQIPLCANLVPVPITNAT |
| | | | | LDRITGKWFYIASAFRN |
| | | | | EEYNKSVQEIQATFFYF |
| | | | | TPNKTEDTIFLREYQTR |
| | | | | QNQCFYNSSYLNVQRE |
| | | | | NGTVSRYEGGREHVAH |
| | | | | LLFLRDTKTLMFGSYLD |
| | | | | DEKNWGLSFYADKPET |
| | | | | TKEQLGEFYEALDCLCI |
| | | | | PRSDVMYTDWKKDKCE |
| | | | | PLEKQHEKERKQEEGES |
| 1000 | APOD | Apolipoprotein | P05090 | MVMLLLLLSALAGLFG |
| | | D | | AAEGQAFHLGKCPNPP |
| | | | | VQENFDVNKYLGRWYE |
| | | | | IEKIPTTFENGRCIQANY |
| | | | | SLMENGKIKVLNQELRA |
| | | | | DGTVNQIEGEATPVNLT |
| | | | | EPAKLEVKFSWFMPSAP |
| | | | | YWILATDYENYALVYS |
| | | | | CTCIIQLFHVDFAWILAR |
| | | | | NPNLPPETVDSLKNILTS |
| | | | | NNIDVKKMTVTDQVNC |
| | | | | PKLS |
| 1001 | TRFE | Serotransferrin | P02787 | MRLAVGALLVCAVLGL |
| | | | | CLAVPDKTVRWCAVSE |
| | | | | HEATKCQSFRDHMKSVI |
| | | | | PSDGPSVACVKKASYLD |
| | | | | CIRAIAANEADAVTLDA |
| | | | | GLVYDAYLAPNNLKPV |
| | | | | VAEFYGSKEDPQTFYYA |
| | | | | VAVVKKDSGFQMNQLR |
| | | | | GKKSCHTGLGRSAGWN |
| | | | | IPIGLLYCDLPEPRKPLE |
| | | | | KAVANFFSGSCAPCAD |
| | | | | GTDFPQLCQLCPGCGCS |
| | | | | TLNQYFGYSGAFKCLK |
| | | | | DGAGDVAFVKHSTIFEN |
| | | | | LANKADRDQYELLCLD |
| | | | | NTRKPVDEYKDCHLAQ |
| | | | | VPSHTVVARSMGGKED |
| | | | | LIWELLNQAQEHFGKD |
| | | | | KSKEFQLFSSPHGKDLL |
| | | | | FKDSAHGFLKVPPRMD |
| | | | | AKMYLGYEYVTAIRNL |
| | | | | REGTCPEAPTDECKPVK |
| | | | | WCALSHHERLKCDEWS |
| | | | | VNSVGKIECVSAETTED |
| | | | | CIAKIMNGEADAMSLD |
| | | | | GGFVYIAGKCGLVPVLA |
| | | | | ENYNKSDNCEDTPEAG |
| | | | | YFAIAVVKKSASDLTW |
| | | | | DNLKGKKSCHTAVGRT |
| | | | | AGWNIPMGLLYNKINH |
| | | | | CRFDEFFSEGCAPGSKK |
| | | | | DSSLCKLCMGSGLNLCE |
| | | | | PNNKEGYYGYTGAFRC |
| | | | | LVEKGDVAFVKHQTVP |
| | | | | QNTGGKNPDPWAKNLN |
| | | | | EKDYELLCLDGTRKPVE |
| | | | | EYANCHLARAPNHAVV |
| | | | | TRKDKEACVHKILRQQ |
| | | | | QHLFGSNVTDCSGNFCL |
| | | | | FRSETKDLLFRDDTVCL |
| | | | | AKLHDRNTYEKYLGEE |
| | | | | YVKAVGNLRKCSTSSLL |
| | | | | EACTFRRP |

TABLE 78
Glycopeptides Associated with NSCLC

| | | | Linking | Linking | |
|---|---|---|---|---|---|
| | | | Site | Site | |
| | Peptide | | Pos. in | Pos. in | Glycan |
| SEQ ID | Structure | Peptide | Protein | Peptide | Structure |
| NO. | (PS) NAME | Sequence | Sequence | Sequence | GL NO. |
| 1002 | A1AT_271_5401 | YLGNATAIFFLPDEGK | 271 | 4 | 5401 |
| 1003 | A1AT_271_5402 | YLGNATAIFFLPDEGK | 271 | 4 | 5402 |
| 1004 | AGP1 | YVGGQEHFAHLLILR | n/a | n/a | n/a |
| 1005 | AGP1_93_7612 | QDQCIYNTTYLNVQR | 93 | 7 | 7612 |
| 1006 | AGP12* | TEDTIFLR | n/a | n/a | n/a |
| 1007 | APOD_98_5412 | ADGTVNQIEGEATPVN | 98 | 16 | 5412 |
| | | LTEPAK | | | |
| 1008 | TRFE_630_5401 | QQQHLFGSNVTDCSG | 630 | 9 | 5401 |
| | | NFCLFR | | | |

TABLE 79
Protein abbreviation, glycosylation site, glycan structure, precursor
ion m/z, and product ion m/z for transitions associated with NSCLC

| SEQ | | Collision | 1^{st} | 1^{st} | 2^{nd} | 2^{nd} | 1^{st} | 2^{nd} |
|---|---|---|---|---|---|---|---|---|
| ID | RT | Energy | Precursor | Precursor | Precursor | Precursor | Product | Product |
| NO. | (min) | (V) | m/z | Charge | m/z | Charge | m/z | m/z |
| 1002 | 37.5 | 30 | 1224.5 | 3 | N/A | N/A | 366.1 | 980 |
| 1003 | 38.2 | 24 | 991.2 | 4 | N/A | N/A | 366.1 | 980.5 |
| 1004 | 28.2 | 33 | 877 | 2 | N/A | N/A | 745.9 | 982.6 |
| 1005 | 22.6 | 31 | 1250.3 | 4 | N/A | N/A | 366.1 | N/A |
| 1006 | 21.9 | 15 | 497.8 | 2 | N/A | N/A | 764.4 | 649.4 |
| 1007 | 24.1 | 30 | 1152.5 | 4 | N/A | N/A | 274.1 | N/A |
| 1008 | 31.4 | 27 | 1108.4 | 4 | N/A | N/A | 366.1 | 1359.6 |

TABLE 80
Glycan Structures

| Glycan Structure | | |
|---|---|---|
| GL NO. | Symbol Structure | Composition |
| 5401 | [symbol] | Hex(5)HexNAc(4)Fuc(0)NeuAc(1) |
| 5402 | [symbol] | Hex(5)HexNAc(4)Fuc(0)NeuAc(2) |
| 5412 | [symbol] | Hex(5)HexNAc(4)Fuc(1)NeuAc(2) |
| 7612 | [symbol] | Hex(7)HexNAc(6)Fuc(1)NeuAc(2) |

Legend for Table 80
● Glc [symbol]
Gal [symbol]
Man [symbol]
Fuc [symbol]
Neu5Ac
▪ GlcNAc [symbol]
GalNAc [symbol]
ManNAc

In some embodiments, a sample from a patient is analyzed by MS and the results are used to determine the presence, absolute amount, and/or relative amount of a glycopeptide consisting of an amino acid sequence selected from SEQ ID NO: 1002-1008 in the sample. In some embodiments, a sample from a patient is analyzed by MS and the results are used to determine the presence, absolute amount, and/or relative amount of a glycopeptide consisting essentially of an amino acid sequence selected from SEQ ID NO: 1002-1008 in the sample. In some embodiments, a sample from a patient is analyzed by MS and the results are used to determine the presence, absolute amount, and/or relative amount of a glycopeptide consisting of, or consisting essentially of, an amino acid sequence selected from SEQ ID NO: 1002-1008 in the sample. In some embodiments, a sample from a patient is analyzed by MS and the results are used to determine the presence, absolute amount, and/or relative amount of a glycopeptide consisting of, or consisting essentially of, an amino acid sequence selected from SEQ ID NO: 1002-1008 in the sample. In some embodiments, the presence, absolute amount, and/or relative amount of a glycopeptide is determined by analyzing the MS results. In some embodiments, the MS results are analyzed using machine-learning. Provided herein are biomarkers selected from glycans, peptides, glycopeptides, fragments thereof, and combinations thereof. In some embodiments, the glycopeptide comprise an amino acid sequence selected from SEQ ID NO: 1002-1008. In some embodiments, the glycopeptide consists essentially of an amino acid sequence selected from SEQ ID NO: 1002-1008.

### O-Glycosylation

In some examples, the glycopeptides set forth herein include O-glycosylated peptides. These peptides include glycopeptides in which a glycan is bonded to the peptide through an oxygen atom of an amino acid. Typically, the amino acid to which the glycan is bonded is threonine (T) or serine (S). In some examples, the amino acid to which the glycan is bonded is threonine (T). In some examples, the amino acid to which the glycan is bonded is serine (S).

### N-Glycosylation

In some examples, the glycopeptides set forth herein include N-glycosylated peptides. These peptides include glycopeptides in which a glycan is bonded to the peptide through a nitrogen atom of an amino acid. Typically, the amino acid to which the glycan is bonded is asparagine (N) or arginine (R). In some examples, the amino acid to which the glycan is bonded is asparagine (N). In some examples, the amino acid to which the glycan is bonded is arginine (R).
In certain examples, the N-glycosylated peptides include members selected from the group consisting of Alpha-1-antitrypsin (AlAT), Alpha-1-acid glycoprotein 1 & 2 (AGP12), Alpha-1-acid glycoprotein 1 (AGP1), Alpha-1-acid glycoprotein 2 (AGP2), Apolipoprotein D (APOD), Serotransferrin (TRFE), and combinations thereof.

### Peptides and Glycopeptides

In some examples, set forth herein is a glycopeptide or peptide consisting of an amino acid sequence selected from the group consisting of SEQ ID NO: 1002-1008.

In some examples, set forth herein is a glycopeptide or peptide consisting essentially of an amino acid sequence selected from the group consisting of SEQ ID NO: 1002-1008.

In some examples, set forth herein is a glycopeptide or peptide comprising an amino acid sequence selected from the group consisting of SEQ ID NO: 1002-1008. In some embodiments, the glycopeptide or peptide is between about 5 to about 50 amino acids in length. In some embodiments, the glycopeptide or peptide is between about 5 to about 20 amino acids in length. In some embodiments, the glycopeptide or peptide is between about 5 to about 15 amino acids in length.

### III. METHODS

Provided herein are methods of identifying the glycoproteomic biomarkers and signatures that may be used to predict which cancer patients respond to pembrolizumab therapy, and have an improvement or a positive change in their condition.

In some embodiments, individual glycopeptide expression levels are associated with various timepoints to determine which glycopeptides changed with events, such as death or metastasis, at the various timepoints. In some embodiments, individual glycopeptide expression levels are associated with time from treatment initiation to death (overall survival, OS) in the patient cohorts.

In some embodiments, multivariable models are used to predict OS in cancer patients. In some embodiments, the cancer patients have NSCLC. In some embodiments, a small subset of glycopeptides for modeling are selected, a model is built and trained based on a training subset of patient data, and a survival score is predicted for each patient based on the trained model. In some embodiments, the resulting scores are dichotomized at a cutoff which optimizes Harrell's C-index. In some embodiments, the resulting scores are dichotomized at a cutoff which optimizes hazard ratio.

In some embodiments, hazard ratio (HR), p-value, and interaction P-value were calculated. In some embodiments, hazard ratio (HR) is calculated from a Cox Proportional Hazards model, representing the multiplicative increase in odds of death for each increase of the biomarker by 1 unit. In some embodiments, p-value is associated with the HR above. In some embodiments, P<0.01 was considered significant. In some embodiments, P<0.05, P<0.01, P<0.005, or P<0.001 was considered significant.

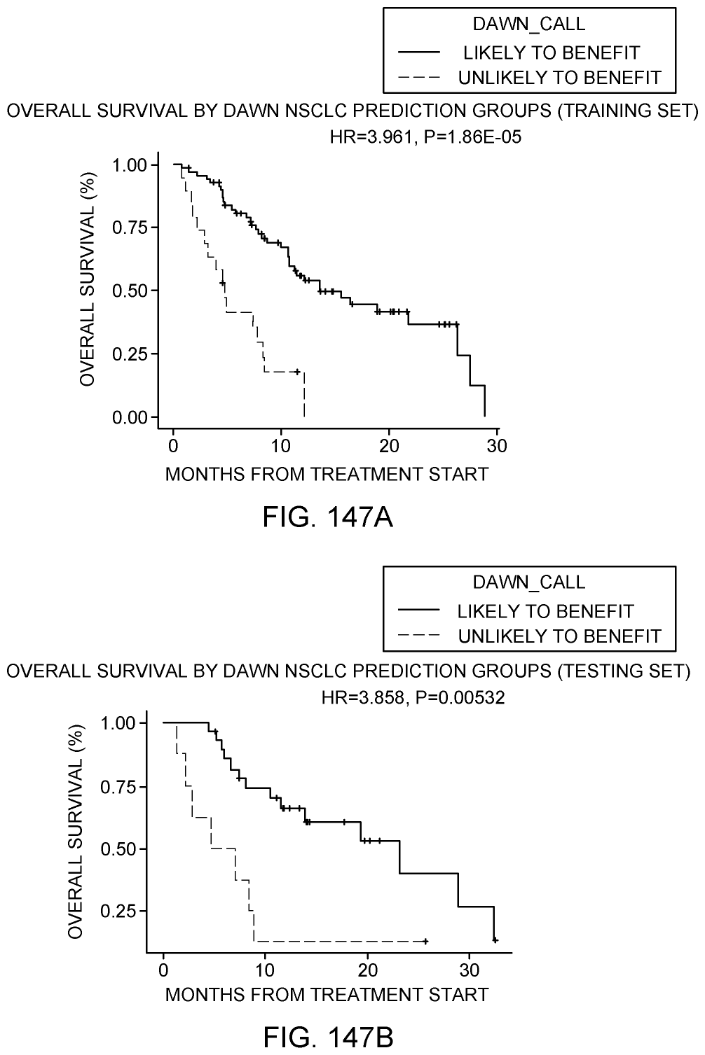

In some embodiments, the model helped to determine whether the glycopeptide marker individually is predictive of OS. In some embodiments, the model helped to determine whether the glycopeptide marker individually is of use in treatment selection or varied with and without treatment. In some embodiments, individual Kaplan-Meier (KM) curves are plotted for the markers relevant in each disease for each outcome, such as OS. In some embodiments, hazard ratios and p-values on the plots are representative of the plotted high/low split at median biomarker expression. Examples of such multivariate KM curves generated from the individual KM curves are seen in FIGS. 147A and 147B.

### Methods for Detecting Glycopeptides

In some embodiments, provided herein are methods for detecting one or more a multiple-reaction-monitoring (MRM) transition, comprising: obtaining a biological sample from a patient, wherein the biological sample comprises one or more glycopeptides; digesting and/or fragmenting a glycopeptide in the sample; and detecting a multiple-reaction-monitoring (MRM) transition.

In some embodiments, provided herein are methods of detecting one or more glycopeptides, wherein each glycopeptide is individually in each instance selected from a glycopeptide consisting of an amino acid sequence selected from the group consisting of SEQ ID NO: 1002-1008. In some embodiments, provided herein are methods of detecting one or more glycopeptides, wherein each glycopeptide is individually in each instance selected from a glycopeptide consisting essentially of an amino acid sequence selected from the group consisting of SEQ ID NO: 1002-1008.

In some embodiments, provided herein are methods of detecting one or more glycopeptides. In some examples, set forth herein is a method of detecting one or more glycopeptide fragments. In certain examples, the method includes detecting the glycopeptide group to which the glycopeptide, or fragment thereof, belongs. In some of these examples, the glycopeptide group is selected from Alpha-1-antitrypsin (AlAT), Alpha-1-acid glycoprotein 1 & 2 (AGP12), Alpha-1-acid glycoprotein 1 (AGP1), Alpha-1-acid glycoprotein 2 (AGP2), Apolipoprotein D (APOD), Serotransferrin (TRFE), and combinations thereof.

In some embodiments, provided herein are methods comprising detecting a glycopeptide, a glycan on the glycopeptide and the glycosylation site residue where the glycan bonds to the glycopeptide. In some embodiments, the method includes detecting a glycan residue. In some embodiments, the method includes detecting a glycosylation site on a glycopeptide. In some embodiments, this process is accomplished with mass spectroscopy used in tandem with liquid chromatography.

In some embodiments, provided herein are methods comprising obtaining a biological sample from a patient. In some examples, the biological sample is synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, bone marrow, gastric acid, bile, semen, pus, aqueous humor, transudate, or combinations of the foregoing. In some examples, the biological sample is selected from the group consisting of blood, plasma, saliva, mucus, urine, stool, tissue, sweat, tears, hair, or a combination thereof. In some examples, the biological sample is a blood sample. In some examples, the biological sample is a plasma sample. In some examples, the biological sample is a saliva sample. In some examples, the biological sample is a mucus sample. In some examples, the biological sample is a urine sample. In some examples, the biological sample is a stool sample. In some examples, the biological sample is a sweat sample. In some examples, the biological sample is a tear sample. In some examples, the biological sample is a hair sample.

In some examples, the method comprises digesting and/or fragmenting a glycopeptide in the sample. In some examples, the method includes digesting a glycopeptide in the sample. In some examples, the method includes fragmenting a glycopeptide in the sample. In some examples, the digested or fragmented glycopeptide is analyzed using mass spectroscopy. In some examples, the glycopeptide is digested or fragmented in the solution phase using digestive enzymes. In some examples, the glycopeptide is digested or fragmented in the gaseous phase inside a mass spectrometer, or the instrumentation associated with a mass spectrometer. In some examples, the mass spectroscopy results are analyzed using machine-learning algorithms. In some examples, the mass spectroscopy results are the quantification of the glycopeptides, glycans, peptides, and fragments thereof. In some examples, this quantification is used as an input in a trained model to generate an output probability. The output probability is a probability of being within a given category or classification, e.g., the classification of being likely to respond to pembrolizumab therapy or not being likely to respond to pembrolizumab therapy.

In some examples, the mass spectrometry is performed using multiple reaction monitoring (MRM) mode. In some examples, the mass spectrometry is performed using qTOF MS in data-dependent acquisition. In some examples, the mass spectrometry is performed using MS-only mode.

In some examples, the method comprises introducing the sample, or a portion thereof, into a mass spectrometer. In some examples, the method comprises fragmenting a glycopeptide in the sample after introducing the sample, or a portion thereof, into the mass spectrometer. In some examples, the method includes digesting a glycopeptide in the sample occurs before introducing the sample, or a portion thereof, into the mass spectrometer. In some examples, the method comprises fragmenting a glycopeptide in the sample to provide a glycopeptide ion, a peptide ion, a glycan ion, a glycan adduct ion, or a glycan fragment ion. In some examples, the method comprises digesting and/or fragmenting a glycopeptide in the sample to provide one or more glycopeptides consisting of an amino acid sequence selected from the group consisting of SEQ ID NO: 1002-1008. In some examples, the method comprises digesting and/or fragmenting a glycopeptide in the sample to provide one or more glycopeptides consisting essentially of an amino acid sequence selected from the group consisting of SEQ ID NO: 1002-1008.

In some examples, the method includes detecting an MRM transition indicative of a glycopeptide or glycan residue, wherein the glycopeptide consists essentially of an amino acid sequence selected from the group consisting of SEQ ID NO: 1002-1008. In some examples, the method includes detecting an MRM transition indicative of a glycopeptide consisting essentially of an amino acid sequence selected from the group consisting of SEQ ID NO: 1002-1008. In some examples, the method includes detecting more than one MRM transition indicative of a combination of glycopeptides having amino acid sequences selected from a combination of SEQ ID NO: 1002-1008.

In some examples, the method includes detecting a MRM transition indicative of a glycopeptide or glycan residue, wherein the glycopeptide consists essentially of an amino acid sequence selected from the group consisting of SEQ ID NO: 1002-1008. In some examples, the method includes detecting a MRM transition indicative of a glycopeptide consisting essentially of an amino acid sequence selected from the group consisting of SEQ ID NO: 1002-1008.

In some examples, the method includes detecting a MRM transition indicative of a glycopeptide or glycan residue, wherein the glycopeptide consists essentially of an amino acid sequence selected from the group consisting of SEQ ID NOs: 1002-1008.

In some examples, the method comprises performing mass spectroscopy on the biological sample using multiple-reaction-monitoring mass spectroscopy (MRM-MS).

In some examples, the method includes digesting a glycoprotein in the sample to provide one or more glycopeptides consisting essentially of an amino acid sequence selected from the group consisting of SEQ ID NO: 1002-1008. In some examples, the biological sample is combined with chemical reagents. In some examples, the biological sample is combined with enzymes. In some examples, the enzymes are lipases. In some examples, the enzymes are proteases. In some examples, the enzymes are serine proteases. In some examples, the enzyme is selected from the group consisting of trypsin, chymotrypsin, thrombin, elastase, and subtilisin. In some examples, the enzyme is trypsin. In some examples, the methods comprises contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, the method includes detecting an MRM transition indicative of a glycopeptide or glycan residue, wherein the glycopeptide consisting of, or consisting essentially of, an amino acid sequence selected from the group consisting of SEQ ID NO: 1002-1008. In some examples, the method includes detecting an MRM transition indicative of a glycopeptide or glycan residue, wherein the glycopeptide consists essentially of an amino acid sequence selected from the group consisting of SEQ ID NO: 1002-1008. In some examples, the method includes detecting an MRM transition indicative of a glycopeptide consisting essentially of an amino acid sequence selected from the group consisting of SEQ ID NO: 1002-1008. In some examples, the method includes detecting more than one MRM transition indicative of a combination of glycopeptides having amino acid sequences selected from a combination of SEQ ID NO: 1002-1008.

In some examples, the method includes detecting a MRM transition indicative of a glycopeptide or glycan residue, wherein the glycopeptide consists essentially of an amino acid sequence selected from the group consisting of SEQ ID NO: 1002-1008.

In some examples, the method comprises performing mass spectrometry on the biological sample using multiple-reaction-monitoring mass spectrometry (MRM-MS).

In some examples, the method comprises digesting a glycopeptide in the sample to provide a glycopeptide consisting of an amino acid sequence selected from the group consisting of SEQ ID NO: 1002-1008. In some examples, the biological sample is contacted with one or more chemical reagents. In some examples, the biological sample is contacted with one or more enzymes. In some examples, the enzymes are lipases. In some examples, the enzymes are proteases. In some examples, the enzymes are serine proteases. In some examples, the enzyme is selected from the group consisting of trypsin, chymotrypsin, thrombin, elastase, and subtilisin. In some of these examples, the enzyme is trypsin. In some examples, the methods include contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, the method includes conducting tandem liquid chromatography-mass spectrometry on the biological sample. In some examples, the method includes conducting multiple-reaction-monitoring mass spectrometry (MRM-MS) on the biological sample. In some examples, the method includes detecting an MRM transition using a triple quadrupole (QQQ) and/or a quadrupole time-of-flight (qTOF) mass spectrometer. In some examples, the method includes detecting an MRM transition using a QQQ mass spectrometer. In some examples, the method includes detecting using a qTOF mass spectrometer. In some examples, a suitable instrument for use with the instant methods is an Agilent 6495B Triple Quadrupole LC/MS. In some examples, the method includes detecting using a QQQ mass spectrometer. In some examples, a suitable instrument for use with the instant methods is an Agilent 6545 LC/Q-TOF.

In some examples, the method comprises detecting more than one MRM transition using a QQQ and/or qTOF mass spectrometer. In some examples, the method includes detecting more than one MRM transition using a QQQ mass spectrometer. In some examples, the method includes detecting more than one MRM transition using a qTOF mass spectrometer. In some examples, the method includes detecting more than one MRM transition using a QQQ mass spectrometer.

In some examples, the methods herein include quantifying one or more glycomic parameters of the one or more biological samples comprises employing a coupled chromatography procedure. In some examples, these glycomic parameters include the identification of a glycopeptide group, identification of glycans on the glycopeptide, identification of a glycosylation site, identification of part of an amino acid sequence which the glycopeptide includes. In some examples, the coupled chromatography procedure comprises: performing or effectuating a liquid chromatography-mass spectrometry (LC-MS) operation. In some examples, the coupled chromatography procedure comprises: performing or effectuating a multiple reaction monitoring mass spectrometry (MRM-MS) operation. In some examples, the methods herein include a coupled chromatography procedure which comprises: performing or effectuating a liquid chromatography-mass spectrometry (LC-MS) operation; and effectuating a multiple reaction monitoring mass spectrometry (MRM-MS) operation. In some examples, the methods include training a machine-learning algorithm using one or more glycomic parameters of the one or more biological samples obtained by one or more of a triple quadrupole (QQQ) mass spectrometry operation and/or a quadrupole time-of-flight (qTOF) mass spectrometry operation. In some examples, the methods include training a machine-learning algorithm using one or more glycomic parameters of the one or more biological samples obtained by a triple quadrupole (QQQ) mass spectrometry operation. In some examples, the methods include training a machine-learning algorithm using one or more glycomic parameters of the one or more biological samples obtained by a quadrupole time-of-flight (qTOF) mass spectrometry operation. In some examples, the methods include quantifying one or more glycomic parameters of the one or more biological samples comprises employing one or more of a triple quadrupole (QQQ) mass spectrometry operation and a quadrupole time-of-flight (qTOF) mass spectrometry operation. In some examples, machine-learning algorithms are used to quantify these glycomic parameters. In some examples, including any of the foregoing, the mass spectrometry is performed using multiple reaction monitoring (MRM) mode. In some examples, the mass spectrometry is performed using qTOF MS in data-dependent acquisition. In some examples, the mass spectrometry is performed using MS-only mode.

In some examples, the method includes detecting one or more MRM transitions indicative of glycans. In some examples, the method comprises quantifying a glycan. In some examples, the method comprises quantifying a first glycan and quantifying a second glycan; and further comprising comparing the quantification of the first glycan with the quantification of the second glycan. In some examples, the method comprises associating the detected glycan with a peptide residue site, whence the glycan was bonded. In some examples, the method comprises generating a glycosylation profile of the sample. In some examples, the method comprises associating the detected glycan with a timepoint.

In some examples, the method includes spatially profiling glycans on a tissue section associated with the sample. In some examples, including any of the foregoing, the method includes spatially profiling glycopeptides on a tissue section associated with the sample. In some examples, the method includes matrix-assisted laser desorption ionization time-of-flight (MALDI-TOF) mass spectrometry in combination with the methods herein.

In some examples, the method includes quantifying relative abundance of a glycan and/or a peptide.

In some examples, the method includes normalizing the amount of a glycopeptide by quantifying a glycopeptide consisting essentially of an amino acid sequence selected from the group consisting of SEQ ID NO: 1002-1008 and comparing that quantification to the amount of another chemical species. In some examples, the method includes normalizing the amount of a peptide by quantifying a glycopeptide consisting of an amino acid sequence selected from the group consisting of SEQ ID NO: 1002-1008, and comparing that quantification to the amount of another glycopeptide consisting of an amino acid sequence selected from the group consisting of SEQ ID NO: 1002-1008. In some examples, the method includes normalizing the amount of a peptide by quantifying a glycopeptide consisting essentially of an amino acid sequence selected from the group consisting of SEQ ID NO: 1002-1008, and comparing that quantification to the amount of another glycopeptide consisting essentially of an amino acid sequence selected from the group consisting of SEQ ID NO: 1002-1008.

### Methods for Classifying Samples Comprising Glycopeptides

In some embodiment, provided herein are methods for identifying a classification for a sample, the method comprising: quantifying by mass spectrometry (MS) one or more glycopeptides in a sample wherein the glycopeptides each, individually in each instance, comprises a glycopeptide consisting essentially of an amino acid sequence selected from the group consisting of, or consisting essentially of SEQ ID NO: 1002-1008; and inputting the quantification into a trained model to generate a output probability; determining if the output probability is above or below a threshold for a classification; and identifying a classification for the sample based on whether the output probability is above or below a threshold for a classification.

In some examples, set forth herein is a method for classifying a biological sample from a patient with respect to a plurality of states associated with responsiveness to pembrolizumab therapy the method comprising: obtaining a biological sample from the patient; performing mass spectrometry of the biological sample using MRM-MS with a QQQ and/or qTOF spectrometer to detect one or more glycopeptides consisting of, or consisting essentially of, an amino acid sequence selected from the group consisting of SEQ ID NO: 1002-1008; analyzing the detected glycopeptides or the MRM transitions to identify a diagnostic classification.

In some embodiments, provided herein are methods of classifying a biological sample from a subject with NSCLC with respect to a plurality of states associated with responsiveness to pembrolizumab therapy the method comprising receiving peptide structure data, inputting quantification data from the peptide structure data into a machine-learning model, identifying, by the machine-learning model a risk score, and classifying the biological sample with respect to a plurality of states based upon the risk score. In some embodiments, the classification is likelihood of response or likelihood of non-response to pembrolizumab. In some embodiments, the classification is likely to benefit from pembrolizumab therapy or not likely to benefit from pembrolizumab therapy. In some embodiments, the risk score represents the likelihood that a subject who is not likely to benefit from the pembrolizumab therapy has an increased risk of death due to administration of pembrolizumab therapy. Ins some embodiments, the risk score represents a risk to the subject of receiving pembrolizumab therapy. In some embodiments, the risk score represents a risk to the subject of not receiving pembrolizumab therapy.

In some embodiments, the method further comprises producing a treatment output. In some embodiments, the treatment output is based upon the classification of likely response to pembrolizumab or likely nonresponse to pembrolizumab. In some embodiments, the treatment output is based upon a risk score for the subject. In some embodiments, a risk score above a certain threshold is associated with a particular treatment output and a risk score below a certain threshold is associated with a particular treatment output.

In some examples, provided herein are methods for identifying glycopeptide biomarkers, comprising: obtaining a biological sample from a patient; digesting and/or fragmenting a glycopeptide in the sample; detecting a multiple-reaction-monitoring (MRM) transition; and classifying the glycopeptides based on the MRM transitions detected. In some examples, a machine-learning algorithm is used to train a model using the MRM transitions as inputs. In some examples, a machine-learning algorithm is trained using the MRM transitions as a training data set. In some examples, the methods herein include identifying glycopeptides, peptides, and glycans based on their mass spectrometry relative abundance. In some examples, a machine-learning algorithm or algorithms select and/or identify peaks in a mass spectrometry spectrum. In some examples, the MS is MRM-MS with a QQQ and/or qTOF mass spectrometer.

In some examples, including any of the foregoing, the mass spectrometry is performed using multiple reaction monitoring (MRM) mode. In some examples, the mass spectrometry is performed using qTOF MS in data-dependent acquisition mode. In some examples, the mass spectrometry is performed using MS-only mode.

In some examples, the machine-learning algorithm is selected from the group consisting of a deep learning algorithm, a neural network algorithm, an artificial neural network algorithm, a supervised machine-learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof. In certain examples, the machine-learning algorithm is a generalized additive model (GAM) regression. For reference, GAM can have relationships between the individual predictors and the dependent variable to provide smooth patterns that can be linear or nonlinear.

In some examples, the method includes classifying a sample as within, or embraced by, a disease classification or a disease severity classification.

In some examples, the classification is identified with 80% confidence, 85% confidence, 90% confidence, 95% confidence, 99% confidence, or 99.9999% confidence.

In some examples, the method includes quantifying by MS the glycopeptide in a sample at a first time point; quantifying by MS the glycopeptide in a sample at a second time point; and comparing the quantification at the first time point with the quantification at the second time point.

In some examples, the method includes quantifying by MS a different glycopeptide in a sample at a third time point; quantifying by MS the different glycopeptide in a sample at a fourth time point; and comparing the quantification at the fourth time point with the quantification at the third time point.

In some examples, the method includes monitoring the health status of a patient.

In some examples, monitoring the health status of a patient includes monitoring the onset and progression of disease in a patient with risk factors such as genetic mutations, as well as detecting cancer recurrence.

In some examples, the method includes diagnosing a patient with a disease or condition based on the quantification. In some examples, the method includes treating the patient with a therapeutically effective amount of a therapeutic agent comprising one or more of a chemotherapeutic, an immunotherapy, a hormone therapy, a targeted therapy, a neoadjuvant therapy, and surgery. In some embodiments, the treatment comprises checkpoint inhibitors. In some examples, the method includes diagnosing an individual with a disease or condition based on the quantification. In some examples, the method includes treating the individual with a therapeutically effective amount of a treatment.

### Methods for Treatment Management of Patients

In some examples, provided herein are methods for managing treatment of a subject diagnosed with NSCLC comprising measuring by mass spectrometry a glycopeptide in a sample from the patient.

In another embodiment, provided herein are methods for managing treatment of a patient having NSCLC; the method comprising: obtaining a biological sample from the patient; performing mass spectrometry of the biological sample using MRM-MS with a QQQ and/or qTOF spectrometer to detect and quantify one or more glycopeptides consisting essentially of an amino acid sequence selected from the group consisting of SEQ ID NO: 1002-1008; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and managing treatment of the patient as having NSCLC based on the classification.

In another embodiment, set forth herein is a method for managing treatment of a subject with NSCLC inputting the quantification of detected glycopeptides or MRM transitions into a trained model to generate an output probability, determining if the output probability is above or below a threshold for a classification; and identifying a diagnostic classification for the patient based on whether the output probability is above or below a threshold for a classification; and managing treatment of the patient as based on the classification. In some examples, the method includes obtaining a biological sample from the subject; performing mass spectrometry of the biological sample using MRM-MS with a QQQ and/or qTOF spectrometer to detect and quantify one or more glycopeptides consisting essentially of an amino acid sequence selected from the group consisting of SEQ ID NO: 1002-1008.

In some examples, set forth herein is a method for managing treatment of a patient having NSCLC; the method comprising: obtaining a biological sample from the patient; performing mass spectrometry of the biological sample using MRM-MS with a QQQ and/or qTOF spectrometer to detect one or more glycopeptides consisting of, or consisting essentially of, an amino acid sequence selected from the group consisting of SEQ ID NO: 1002-1008; analyzing the detected glycopeptides or the MRM transitions to identify a classification; and managing treatment of the patient based on the diagnostic classification.

In some examples, set forth herein is a method for managing treatment of a patient having NSCLC; the method comprising: analyzing detected or quantified glycopeptides or MRM transitions to identify a classification; and managing treatment of the patient based on the classification. In some examples, the method includes obtaining a biological sample from the patient; and performing mass spectrometry of the biological sample using MRM-MS with a QQQ and/or qTOF spectrometer to detect one or more glycopeptides consisting of, or consisting essentially of, an amino acid sequence selected from the group consisting of SEQ ID NO: 1002-1008.

In some embodiments, provided herein are methods for determining whether a subject diagnosed with non-small-cell lung cancer (NSCLC) will benefit from pembrolizumab therapy comprising detecting one or more biomarkers. In some embodiments, the one or more biomarkers comprise one or more glycopeptides. In some embodiments, the one or more biomarkers comprises one or more peptide structures set forth in Table 78. In some embodiments, the method comprises detecting one or more glycopeptides comprising a sequence set forth in SEQ ID NO: 1002-1008. In some embodiments, the method comprises detecting two or more glycopeptides comprising a sequence set forth in SEQ ID NO: 1002-1008. In some embodiments, the method comprises detecting three or more glycopeptides comprising a sequence set forth in SEQ ID NO: 1002-1008. In some embodiments, the method comprises detecting four or more glycopeptides comprising a sequence set forth in SEQ ID NO: 1002-1008. In some embodiments, the method comprises detecting five or more glycopeptides comprising a sequence set forth in SEQ ID NO: 1002-1008. In some embodiments, the method comprises detecting six or more glycopeptides comprising a sequence set forth in SEQ ID NO: 1002-1008. In some embodiments, the method comprises detecting each of the glycopeptides comprising a sequence set forth in SEQ ID NO: 1002-1008. In some embodiments, the glycopeptide comprises a glycan with the structures in Table 78. In some embodiments, the glycopeptide is a glycopeptide of a glycoprotein provided in Table 77. In some embodiments, the glycopeptide is a glycopeptide of a glycoprotein comprising a sequence set forth in SEQ ID NO: 997-1001.

In some embodiments, the determination of whether a subject diagnosed with NSCLC will benefit from pembrolizumab therapy is based upon presence and/or amount of at least one, at least two, at least three, at least four, at least five, at least six, or seven peptide structures from Table 78. In some embodiments, the determination is based upon the presence and/or amount of one or more peptides comprising the amino acid sequence of SEQ ID NO: 1002-1008. In some embodiments, the determination is based upon the presence and/or amount of two or more peptides comprising the amino acid sequence of SEQ ID NO: 1002-1008. In some embodiments, the determination is based upon the presence and/or amount of three or more peptides comprising the amino acid sequence of SEQ ID NO: 1002-1008. In some embodiments, the determination is based upon the presence and/or amount of four or more peptides comprising the amino acid sequence of SEQ ID NO: 1002-1008. In some embodiments, the determination is based upon the presence and/or amount of five or more peptides comprising the amino acid sequence of SEQ ID NO: 1002-1008. In some embodiments, the determination is based upon the presence and/or amount of six or more peptides comprising the amino acid sequence of SEQ ID NO: 1002-1008. In some embodiments, the determination is based upon the presence and/or amount of each of the peptides comprising the amino acid sequence of SEQ ID NO: 1002-1008. In some embodiments, the glycopeptide is a glycopeptide of a glycoprotein provided in Table 77. In some embodiments, the glycopeptide is a glycopeptide of a glycoprotein comprising a sequence set forth in SEQ ID NO: 1-5.

In some embodiments, the determination is based upon the presence and/or amount of one or more peptides consisting of the amino acid sequence of SEQ ID NO: 1002-1008. In some embodiments, the determination is based upon the presence and/or amount of two or more peptides consisting of the amino acid sequence of SEQ ID NO: 1002-1008. In some embodiments, the determination is based upon the presence and/or amount of three or more peptides consisting of the amino acid sequence of SEQ ID NO: 1002-1008. In some embodiments, the determination is based upon the presence and/or amount of four or more peptides consisting of the amino acid sequence of SEQ ID NO: 1002-1008. In some embodiments, the determination is based upon the presence and/or amount of five or more peptides consisting of the amino acid sequence of SEQ ID NO: 1002-1008. In some embodiments, the determination is based upon the presence and/or amount of six or more peptides consisting of the amino acid sequence of SEQ ID NO: 1002-1008. In some embodiments, the determination is based upon the presence and/or amount of each of the peptides consisting of the amino acid sequence of SEQ ID NO: 1002-1008. In some embodiments, the glycopeptide is a glycopeptide of a glycoprotein provided in Table 77. In some embodiments, the glycopeptide is a glycopeptide of a glycoprotein comprising a sequence set forth in SEQ ID NO: 1-5.

In some embodiments, the determination is based upon the presence and/or amount of one or more peptides comprising the amino acid sequence of SEQ ID NO: 1002-1008. In some embodiments, the determination is based upon the presence and/or amount of two or more peptides comprising the amino acid sequence of SEQ ID NO: 1002-1008. In some embodiments, the determination is based upon the presence and/or amount of three or more peptides comprising the amino acid sequence of SEQ ID NO: 1002-1008. In some embodiments, the determination is based upon the presence and/or amount of four or more peptides comprising the amino acid sequence of SEQ ID NO: 1002-1008. In some embodiments, the determination is based upon the presence and/or amount of five or more peptides comprising the amino acid sequence of SEQ ID NO: 1002-1008. In some embodiments, the determination is based upon the presence and/or amount of six or more peptides comprising the amino acid sequence of SEQ ID NO: 1002-1008. In some embodiments, the determination is based upon the presence and/or amount of each of the peptides comprising the amino acid sequence of SEQ ID NO: 1002-1008. In some embodiments, a peptide comprising the amino acid sequence set forth in SEQ ID NO:8 and/or SEQ ID NO:10 is detected. In some embodiments, the glycopeptide is a glycopeptide of a glycoprotein provided in Table 77. In some embodiments, the glycopeptide is a glycopeptide of a glycoprotein comprising a sequence set forth in SEQ ID NO: 1-5.

In some embodiments, the determination is based upon the presence and/or amount of one or more peptides consisting of the amino acid sequence of SEQ ID NO: 1002-1008. In some embodiments, the determination is based upon the presence and/or amount of two or more peptides consisting of the amino acid sequence of SEQ ID NO: 1002-1008. In some embodiments, the determination is based upon the presence and/or amount of three or more peptides consisting of the amino acid sequence of SEQ ID NO: 1002-1008. In some embodiments, the determination is based upon the presence and/or amount of four or more peptides consisting of the amino acid sequence of SEQ ID NO: 1002-1008. In some embodiments, the determination is based upon the presence and/or amount of five or more peptides consisting of the amino acid sequence of SEQ ID NO: 1002-1008. In some embodiments, the determination is based upon the presence and/or amount of six or more peptides consisting of the amino acid sequence of SEQ ID NO: 1002-1008. In some embodiments, the determination is based upon the presence and/or amount of each of the peptides consisting of the amino acid sequence of SEQ ID NO: 1002-1008. In some embodiments, a peptide comprising the amino acid sequence set forth in SEQ ID NO:1004 and/or SEQ ID NO:1006 is detected. In some embodiments, the glycopeptide is a glycopeptide of a glycoprotein provided in Table 77. In some embodiments, the glycopeptide is a glycopeptide of a glycoprotein comprising a sequence set forth in SEQ ID NO: 997-1001.

### Methods of Treatment

In some embodiments, provided herein are methods of treating a subject with NSCLC. In some embodiments, the methods comprises detecting one or more peptides or glycopeptides provided herein and administering a treatment based upon the presence, absence, or amount of the glycopeptide. In some embodiments, the treatment is pembrolizumab therapy. In some embodiments, a therapy other than pembrolizubam is administered. In some embodiments, the subject is determined to be suitable for therapy with pembrolizumab based upon the peptides and glycopeptides provided herein.

In some embodiments, provided herein is a method of treating non-small-cell lung cancer (NSCLC) in an individual based upon the presence, absence, or amount of one or more peptide structures set forth in Table 78. In some embodiments, provided herein is a method of treating non-small-cell lung cancer (NSCLC) in an individual based upon the presence, absence, or amount of two or more peptide structures set forth in Table 78. In some embodiments, provided herein is a method of treating non-small-cell lung cancer (NSCLC) in an individual based upon the presence, absence, or amount of three or more peptide structures set forth in Table 78. In some embodiments, provided herein is a method of treating non-small-cell lung cancer (NSCLC) in an individual based upon the presence, absence, or amount of four or more peptide structures set forth in Table 78. In some embodiments, provided herein is a method of treating non-small-cell lung cancer (NSCLC) in an individual based upon the presence, absence, or amount of five or more peptide structures set forth in Table 78. In some embodiments, provided herein is a method of treating non-small-cell lung cancer (NSCLC) in an individual based upon the presence, absence, or amount of six or more peptide structures set forth in Table 78. In some embodiments, provided herein is a method of treating non-small-cell lung cancer (NSCLC) in an individual based upon the presence, absence, or amount of each of the peptide structures set forth in Table 78. In some embodiments, one or more peptide structures set forth in SEQ ID NO: 1002-1008 is detected. In some embodiments, two or more peptide structures set forth in SEQ ID NO: 1002-1008 are detected. In some embodiments, three or more peptide structures set forth in SEQ ID NO: 1002-1008 are detected. In some embodiments, four or more peptide structures set forth in SEQ ID NO: 1002-1008 are detected. In some embodiments, five or more peptide structures set forth in SEQ ID NO: 1002-1008 are detected. In some embodiments, six or more peptide structures set forth in SEQ ID NO: 1002-1008 are detected. In some embodiments, each of the peptide structures set forth in SEQ ID NO: 1002-1008 is detected. In some embodiments, a peptide comprising the amino acid sequence set forth in SEQ ID NO:8 and/or SEQ ID NO:10 is detected. In some embodiments, the method further comprises delivering a therapeutic agent based upon the presence, absence, or amount of one or more peptide structures set forth in Table 78. In some embodiments, the method further comprises delivering a therapeutic agent based upon the presence, absence, or amount of two or more peptide structures set forth in Table 78. In some embodiments, the method further comprises delivering a therapeutic agent based upon the presence, absence, or amount of three or more peptide structures set forth in Table 78. In some embodiments, the method further comprises delivering a therapeutic agent based upon the presence, absence, or amount of four or more peptide structures set forth in Table 78. In some embodiments, the method further comprises delivering a therapeutic agent based upon the presence, absence, or amount of five or more peptide structures set forth in Table 78. In some embodiments, the method further comprises delivering a therapeutic agent based upon the presence, absence, or amount of six or more peptide structures set forth in Table 78. In some embodiments, the method further comprises delivering a therapeutic agent based upon the presence, absence, or amount of each of the peptide structures set forth in Table 78. In some embodiments, the method comprises selecting a therapeutic agent based upon the presence, absence, or amount of one or more peptide structures set forth in Table 78. In some embodiments, the method comprises selecting a therapeutic agent based upon the presence, absence, or amount of two or more peptide structures set forth in Table 78. In some embodiments, the method comprises selecting a therapeutic agent based upon the presence, absence, or amount of three or more peptide structures set forth in Table 78. In some embodiments, the method comprises selecting a therapeutic agent based upon the presence, absence, or amount of four or more peptide structures set forth in Table 78. In some embodiments, the method comprises selecting a therapeutic agent based upon the presence, absence, or amount of five or more peptide structures set forth in Table 78. In some embodiments, the method comprises selecting a therapeutic agent based upon the presence, absence, or amount of six or more peptide structures set forth in Table 78. In some embodiments, the method comprises selecting a therapeutic agent based upon the presence, absence, or amount of each of the peptide structures set forth in Table 78. In some embodiments, the therapeutic agent is a chemotherapeutic agent and/or a hormone therapy. In some embodiments, the glycopeptide is a glycopeptide of a glycoprotein provided in Table 77. In some embodiments, the glycopeptide is a glycopeptide of a glycoprotein comprising a sequence set forth in SEQ ID NO: 997-1001.

In some embodiments, the method comprises selecting a therapy to treat NSCLC. In some embodiments, the method of selecting a therapy for NSCLC comprises inputting quantification data identified from peptide structure data for a set of peptides and/or glycopeptides into one or more machine-learning model trained to identify a risk score. In some embodiments, the method of selecting a therapy comprises classifying the sample as being likely to respond or not likely to respond to pembrolizumab based upon the disease indicator. In some embodiments, the therapy is selected based upon presence and/or amount of at least one, at least two, at least three, at least four, at least five, at least six, or seven peptide structures from Table 79. In some embodiments, the therapy is selected based upon presence and/or amount of at least one, at least two, at least three, at least four, at least five, at least six, or seven peptide and/or glycopeptides comprising the amino acid sequence of SEQ ID NO: 1002-1008 along with the associated glycan set forth in Table 79. In some embodiments, the presence, absence, and/or amount of one or more peptides and/or glycopeptide is determined by MRM-MS.

In some embodiments, a therapy other than pembrolizumab is selected and/or administered based upon the presence, absence and/or amount of one or more glycopeptides provided herie] . In some embodiments, the other therapy is selected from the group comprising a surgery, a chemotherapeutic therapy, a patient-specific therapy, a targeted immunotherapy, a radiation procedure, a radiofrequency ablation (RFA) procedure, or a combination thereof. In some embodiments, the surgery comprises the removal of one or more parts of the lung. In some embodiments, the platinum-coordinating compound comprises one of cisplatin, carboplatin, and nedaplatin. In some embodiments, the chemotherapeutic comprises docetaxel, paclitaxel, albumin-bound paclitaxel, vinorelbine, gemcitabine, irinotecan, pemetrexed, tegafur/gimeracil/oteracil, etoposide, or a combination thereof. In some embodiments, the chemotherapeutic therapy is a platinum-doublet regimen. In some embodiments, the chemotherapeutic therapy is a platinum-triple regimen. In some embodiments, the targeted immunotherapy comprises one or more antibody directed towards an immune system checkpoint protein including but not limited to PD-1, PD-L1, and CTLA-4. In some embodiments, the therapy for NSCLC comprises a combination of one or more antibody that targets PD-1, PD-L1, and CTLA-4. In some embodiments, the targeted therapy comprises one or more patient-specific therapy agent selected based on patient-specific changes in tumor cell gene expression including but not limited to changes in KRAS, EGFR, ALK, ROS1, BRAF, RET, MET, and NTRK genes. In some embodiments, the patient-specific therapy is an inhibitor of an oncogene. In some embodiments, the patient-specific therapy is an inhibitor of one or more of KRAS, EGFR, ALK, ROS1, BRAF, MEK, RET, MET, and NTRK. In some embodiments, the radiation procedure comprises the use of high-energy rays or particles to treat NSCLC. In some embodiments, the brachytherapy comprises the placement of radioactive material in or adjacent to the tumor in the airway (e.g., bronchial tubes).

In some embodiments, the subject is administered a combination therapy comprising pembrolizumab and chemotherapy based upon the presence, absence, and/or amount of one or more peptides or glycopeptides provided herein. In some embodiments, the chemotherapy therapy comprises a platinum-coordinating compound, a chemotherapeutic, or a combination thereof. In some embodiments, the platinum-coordinating compound comprises one of cisplatin (CDDP), carboplatin (CBDCA), and nedaplatin (CDGP). In some embodiments, the chemotherapeutic comprises docetaxel (Taxotere, DTX), paclitaxel (Taxol, PTX), albumin-bound paclitaxel (nab-paclitaxel, Abraxane), vinorelbine (Navelbine,VNR), gemcitabine (Gemzar, GEM), irinotecan (CPT-11), pemetrexed (Alimta, PEM), tegafur/gimeracil/oteracil (S1), etoposide (VP-16), or a combination thereof. In some embodiments, the chemotherapeutic therapy is a platinum-doublet regimen. In some embodiments, the chemotherapeutic therapy is a platinum-triple regimen. In some embodiments, the method comprises classifying a biological sample with respect to a plurality of states associated with NSCLC based upon one or more peptide structures provided in Table 79. In some embodiments, the method comprises inputting quantification data identified from peptide structure data for a set of peptides and/or glycopeptides into one or more machine-learning model trained to identify a risk score. In some embodiments, the method comprises classifying the subject as being likely to respond to pembrolizumab or not likely to respond to pembrolizumab based upon the risk score. In some embodiments, the peptide structure data comprises one or more peptide structure provided in Table 79. In some embodiments, the presence, absence, and/or amount of one or more peptides and/or glycopeptides is determined by MRM-MS. In some embodiments, the method comprises selecting a particular therapy described herein based upon the presence, amount, and/or relative amount of one or more biomarkers comprising the peptide structures provided in Table 79. In some embodiments, the method comprises selecting a particular therapy described herein based upon the presence, amount, and/or relative amount of one or more biomarkers comprising the glycopeptides provided in Table 79. In some embodiments, the method comprises administering a particular therapy described herein based upon the presence, amount, and/or relative amount of one or more biomarkers comprising the peptide structures provided in Table 79. In some embodiments, the method comprises administering a particular therapy described herein based upon the presence, amount, and/or relative amount of one or more biomarkers comprising the glycopeptides provided in Table 79. In some embodiments, the method further comprises selecting a particular therapy described herein based upon the disease indicator and/or classification. In some embodiments, the method further comprises administering a particular therapy described herein based upon the disease indicator and/or classification.

In some embodiments, patients are treated with a therapeutically effective amount of an immune-therapeutic. In some embodiments, the immune-therapeutic comprises an immune checkpoint inhibitor. In some embodiments, the checkpoint inhibitor comprises pembrolizumab.

In some embodiments, patients are treated with a therapeutically effective amount of a targeted therapeutic agent. In some embodiments, the targeted therapeutic agent is a drug that targets blood vessel that targets vascular endothelial growth factor (VEGF) such as bevacizumab, ramucirumab, and ziv-aflibercept. In some embodiments, the targeted therapeutic agent comprises an epidermal growth factor receptor (EGFR). In some embodiments, the EGFR comprises cetuximab or panitumumab. In some embodiments, the targeted therapeutic agent comprises a kinase inhibitor. In some embodiments, the kinase inhibitor comprises regorafenib.

In some embodiments, the targeted immunotherapy comprises one or more antibody directed towards an immune system checkpoint protein including but not limited to PD-1, PD-L1, and CTLA-4. In some embodiments, the antibody targeting PD-1 comprises nivolumab (Opdivo), pembrolizumab (Keytruda), and cemiplimab (Libtayo). In some embodiments, the antibody targeting PD-L1 comprises chemotherapy (Tecentriq), durvalumab (Imfinzi), and avelumab (Bavencio). In some embodiments, the antibody targeting CTLA-4 comprises ipilimumab (Yervoy). In some embodiments, the therapy for NSCLC comprises a combination of one or more antibody that targets PD-1, PD-L1, and CTLA-4. In some embodiments, the method comprises classifying a biological sample with respect to a plurality of states associated with NSCLC based upon one or more peptide structures provided in Table 78. In some embodiments, the method comprises classifying a biological sample with respect to a plurality of states associated with NSCLC based upon one or more glycopeptides provided in Table 78. In some embodiments, the method comprises inputting quantification data identified from peptide structure data for a set of peptides and/or glycopeptides into one or more machine-learning model trained to identify a risk score. In some embodiments, the method comprises classifying the sample as being likely to respond to pembrolizumab or not likely to respond to pembrolizumab based upon the risk indicator. In some embodiments, the peptide structure data comprises one or more peptide structure provided in Table 79. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 78. In some embodiments, the method comprises selecting a particular therapy described herein based upon the presence, amount, and/or relative amount of one or more biomarkers comprising the glycopeptides provided in Table 79. In some embodiments, the method comprises administering a particular therapy described herein based upon the presence, amount, and/or relative amount of one or more biomarkers comprising the peptide structures provided in Table 78. In some embodiments, the method comprises administering a particular therapy described herein based upon the presence, amount, and/or relative amount of one or more biomarkers comprising the glycopeptides provided in Table 78. In some embodiments, the method further comprises selecting a particular therapy described herein based upon the risk score and/or classification. In some embodiments, the method further comprises administering a particular therapy described herein based upon the risk score and/or classification.

In some embodiments, the alternative therapy comprises one or more patient-specific therapy agent selected based on patient-specific changes in tumor cell gene expression including but not limited to changes in KRAS, EGFR, ALK, ROS1, BRAF, RET, MET, and NTRK genes. In some embodiments, the patient-specific therapy is an inhibitor of an oncogene. In some embodiments, the patient-specific therapy is an inhibitor of one or more of KRAS, EGFR, ALK, ROS1, BRAF, MEK, RET, MET, and NTRK. In some embodiments, the patient-specific therapy comprises one or more of sotorasib (Lumakras), erlotinib (Tarceva), afatinib (Gilotrif), gefitinib (Iressa), osimertinib (Tagrisso), dacomitinib (Vizimpro), amivantamab (Rybrevant), nobocertinib (Exkivity), necitumumab (Portrazza), crizotinib (Xalkori), ceritinib (Zykadia), alectinib (Alecensa), brigatinib (Alunbrig), lorlatinib (Lorbrena), entrectinib (Rozlytrek), dabrafenib (Tafinlar), trametinib (Mekinist), selpercatinib (Retevmo), pralsetinib (Gavreto), capmatinib (Tabrecta), tepotinib (Tepmetko), larotrectinib (Vitrakvi), and combinations thereof. In some embodiments the patient-specific therapy comprises an angiogenesis inhibitor. In some embodiments, the angiogenesis inhibitor comprises one of bevacizumab (Avastin, BEV) and ramucirumab (Cyramza, RAM). In some embodiments, the therapy for NSCLC comprises a combination of one or more patient-specific therapy agents. In some embodiments, the method comprises classifying a biological sample with respect to a plurality of states associated with NSCLC based upon one or more peptide structures provided in Table 78. In some embodiments, the method comprises classifying a biological sample with respect to a plurality of states associated with NSCLC based upon one or more glycopeptides provided in Table 78. In some embodiments, the method comprises inputting quantification data identified from peptide structure data for a set of peptides and/or glycopeptides into one or more machine-learning model trained to identify a disease indicator. In some embodiments, the method comprises classifying the sample as having NSCLC or not having NSCLC based upon the disease indicator. In some embodiments, the peptide structure data comprises one or more peptide structure provided in Table 78. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 78. In some embodiments, the method comprises selecting a particular therapy described herein based upon the presence, amount, and/or relative amount of one or more biomarkers comprising the glycopeptides provided in Table 79. In some embodiments, the method comprises administering a particular therapy described herein based upon the presence, amount, and/or relative amount of one or more biomarkers comprising the peptide structures provided in Table 78. In some embodiments, the method comprises administering a particular therapy described herein based upon the presence, amount, and/or relative amount of one or more biomarkers comprising the glycopeptides provided in Table 78. In some embodiments, the method further comprises selecting a particular therapy described herein based upon the disease indicator and/or classification. In some embodiments, the method further comprises administering a particular therapy described herein based upon the disease indicator and/or classification.

In some embodiments, the radiation procedure comprises the use of high-energy rays or particles to treat NSCLC. In some embodiments, the radiation procedure comprises external beam radiation therapy (EBRT) and internal radiation therapy (also referred to as brachytherapy). In some embodiments, the EBRT comprises one or more of stereotactic ablative radiotherapy (SABR), three-dimensional conformal radiation therapy (3D-CRT), intensity modulated radiation therapy (IMRT), volumetric modulated arc therapy (VMAT), and stereotactic radiosurgery (SRS). In some embodiments, the brachytherapy comprises the placement of radioactive material in or adjacent to the tumor in the airway (e.g., bronchial tubes). In some embodiments, the method comprises classifying a biological sample with respect to a plurality of states associated with NSCLC based upon one or more peptide structures provided in Table 78. In some embodiments, the method comprises inputting quantification data identified from peptide structure data for a set of peptides and/or glycopeptides into one or more machine-learning model trained to identify a risk score. In some embodiments, the method comprises classifying the sample as being likely to respond to pembrolizumab based upon the risk score. In some embodiments, the peptide structure data comprises one or more peptide structure provided in Table 78. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 78. In some embodiments, the method comprises selecting a particular therapy described herein based upon the presence, amount, and/or relative amount of one or more biomarkers comprising the glycopeptides provided in Table 78. In some embodiments, the method comprises administering a particular therapy described herein based upon the presence, amount, and/or relative amount of one or more biomarkers comprising the peptide structures provided in Table 78. In some embodiments, the method comprises administering a particular therapy described herein based upon the presence, amount, and/or relative amount of one or more biomarkers comprising the glycopeptides provided in Table 78. In some embodiments, the method further comprises selecting a particular therapy described herein based upon the disease indicator and/or classification. In some embodiments, the method further comprises administering a particular therapy described herein based upon the disease indicator and/or classification.

In some embodiments, pembrolizumab comprises a VH comprising the amino acid sequence of SEQ ID NO:1009 and a VL comprising the amino acid sequence of SEQ ID NO:1010. In some embodiments, pembrolizumab is administered as a first line treatment. In some embodiments, the first treatment may comprise an individual with early-stage NSCLC or NSCLC that has not metastasized. In some embodiments, the pembrolizumab is used alone when the individual with NSCLC cannot undergo surgery or chemotherapy. In some embodiments, the pembrolizumab is administered alone when the individual with NSCLC is positive for PD-L1. In some embodiments, the pembrolizumab is administered alone when the individual with NSCLC is negative for EGFR or ALK.

In some embodiments, the pembrolizumab is used in combination with a chemotherapy for treating an individual having NSCLC. In some embodiments, the pembrolizumab is administered with pemetrexed. In some embodiments, pembrolizumab is administered with pemetrexed and a platinum compound. In some embodiments, the platinum compound comprises one or more of cisplatin, carboplatin, and nedaplatin. In some embodiments, the individual having NSCLC has NSCLC that has metastasized. In some embodiments, the individual having NSCLC has nonsquamous NSCLC. In some embodiments, the individual having NSCLC does not have a mutated EGFR or ALK gene.

In some embodiments, the pembrolizumab is administered with carboplatin. In some embodiments, the pembrolizumab is administered with carboplatin and either paclitaxel or paclitaxel protein-bound. In some embodiments, the individual having NSCLC has NSCLC that has metastasized. In some embodiments, the individual having NSCLC has squamous NSCLC.

In some embodiments, the pembrolizumab is administered at a dose of about 200 mg every 3 weeks (Q3W). In some embodiments, the pembrolizumab is administered at a dose of about 400 mg Q3W. In some embodiments, the pembrolizumab is administered at a dose of about 2 mg/kg every 3 weeks (Q3W). In some embodiments, the antibody that specifically binds to PD-1 is administered, optionally as a monotherapy, in a first treatment cycle followed by combination therapy with chemotherapy. In some embodiments, the pembrolizumab is administered at a dose of about 200 mg to about 400 mg Q3W followed by combination therapy with chemotherapy. In some embodiments, the pembrolizumab is administered at a dose of at least 2 mg/kg QW or Q3W followed by combination therapy with chemotherapy. In some embodiments, the pembrolizumab is administered at a dose of about 100 mg, 200 mg, 300 mg, or 400 mg Q3W followed by combination therapy with chemotherapy. In some embodiments, the methods provided herein include treatment of the individual by administration of pembrolizumab, wherein pembrolizumab is a humanized antibody comprising a heavy chain variable region (VH) having the sequence set forth in SEQ ID NO:1009, and a light chain variable region (VL) having the sequence set forth in SEQ ID NO:1010. In some embodiments, the methods provided herein include treatment of the individual by administration of pembrolizumab, wherein the PD-1 antibody is a humanized antibody comprising a heavy chain having the sequence set forth in SEQ ID NO:1011, and a light chain having the sequence set forth in SEQ ID NO:1012. In some embodiments, a complete response (CR), partial response (PR), or stable disease (SD) is achieved following treatment. In some embodiments, the tumor size is reduced following treatment. In some embodiments, the number of lesions are reduced following treatment. In some embodiments, treatment with one or more antibody is continued until the individual is no longer responsive to therapy, is no longer willing to continue therapy, or is unable to continue therapy. In some embodiments, treatment with one or more antibody is continued for about 2 years.

TABLE 81
XIV. Pembrolizumab heavy chain and
light chain variable region amino acid
sequences

| Antibody | Variable | SEQ ID | |
|---|---|---|---|
| ID | Region | NO: | Amino Acid Sequence |
| Pembrolizumab | VH | 1009 | QVQLVQSGVEVKKPGASVK |
| | | | VSCKASGYTFTNYYMYWVR |
| | | | QAPGQGLEWMGGINPSNGG |
| | | | TNFNEKFKNRVTLTTDSST |
| | | | TTAYMELKSLQFDDTAVYY |
| | | | CARRDVHYRFDMGFDYWGQ |
| | | | GTTVTVSS |
| Pembrolizumab | VL | 1010 | EIVLTQSPATLSLSPGERA |
| | | | TLSCRASKGVSTSGYSYLH |
| | | | WYQQKPGQAPRLLIYLASY |
| | | | LESGVPARFSGSGSGTDFT |
| | | | LTISSLEPEDFAVYYCQHS |
| | | | RDLPLTFGGGTKVEIK |

TABLE 82
XV. Pembrolizumab full-length
heavy chain and light chain amino acid
sequences

| Antibody | | SEQ ID | |
|---|---|---|---|
| ID | Chain | NO: | Amino Acid Sequence |
| Pembrolizumab | Heavy | 1011 | QVQLVQSGVEVKKPGASVK |
| | chain | | VSCKASGYTFTNYYMYWVR |
| | | | QAPGQGLEWMGGINPSNGG |
| | | | TNENEKEKNRVTLTTDSST |
| | | | TTAYMELKSLQFDDTAVYY |
| | | | CARRDYRFDMGFDYWGQGT |
| | | | TVTVSSASTKGPSVFPLAP |
| | | | CSRSTSESTAALGCLVKDY |
| | | | FPEPVTVSWNSGALTSGVH |
| | | | TFPAVLQSSGLYSLSSVVT |
| | | | VPSSSLGTKTYTCNVDHKP |
| | | | SNTKVDKRVESKYGPPCPP |
| | | | CPAPEFLGGPSVFLFPPKP |
| | | | KDTLMISRTPEVTCVVVDV |
| | | | SQEDPEVQFNWYVDGVEVH |
| | | | NAKTKPREEQFNSTYRVVS |
| | | | VLTVLHQDWLNGKEYKCKV |
| | | | SNKGLPSSIEKTISKAKGQ |
| | | | PREPQVYTLPPSQEEMTKN |
| | | | QVSLTCLVKGFYPSDIAVE |
| | | | WESNGQPENNYKTTPPVLD |
| | | | SDGSFFLYSRLTVDKSRWQ |
| | | | EGNVFSCSVMHEALHNHYT |
| | | | QKSLSLSLGK |
| Pembrolizumab | Light | 1012 | EIVLTQSPATLSLSPGERA |
| | chain | | TLSCRASKGVSTSGYSYLH |
| | | | WYQQKPGQAPRLLIYLASY |
| | | | LESGVPARFSGSGSGTDFT |
| | | | LTISSLEPEDFAVYYCQHS |
| | | | RDLPLTFGGGTKVEIKRTV |
| | | | AAPSVFIFPPSDEQLKSGT |
| | | | ASVVCLLNNFYPREAKVQW |
| | | | KVDNALQSGNSQESVTEQD |
| | | | SKDSTYSLSSTLTLSKADY |
| | | | EKHKVYACEVTHQGLSSPV |
| | | | TKSFNRGEC |

In some embodiments, the antibody comprises the VH and/or the VL of antibody Pembrolizumab as shown in Table 81. In some embodiments, the antibody comprises the heavy chain and/or the light chain of antibody Pembrolizumab as shown in Table 82.

In some embodiments, the antibody that binds to PD-1 comprises a heavy chain variable domain (VHI) sequence having at least 90%, 91%, 92%, 93%, 94%, 95%, 960%, 97%, 9800, 9900, or 10000 sequence identity to the amino acid sequence of SEQ ID NO: 1009. In certain embodiments, a VH sequence contains substitutions (e.g., conservative substitutions), insertions, or deletions relative to the amino acid sequence of SEQ ID NO: 1009.

In another aspect, an antibody that binds to PD-1 is provided, wherein the antibody comprises a light chain variable domain (VL) having at least 900%, 91%, 9200, 9300 9400, 95%, 96%, 97%, 98%, 99%, or 100% sequence identity to the amino acid sequence of SEQ ID NO: 1010. In certain embodiments, a VL sequence contains substitutions (e.g., conservative substitutions), insertions, or deletions relative to the amino acid sequence of SEQ ID NO:1010.

In one embodiment, the antibody that binds to PD-1 comprises a VH comprising the amino acid sequence of SEQ ID NO:1009 and a VL comprising the amino acid sequence of SEQ ID NO:1010.

In some embodiments, an antibody that binds to PD-1 comprises a heavy chain comprising the amino acid sequence of SEQ ID NO:1011 and a light chain comprising the amino acid sequence of SEQ ID NO:1012.

In another aspect, an antibody that binds to PD-1 is provided, wherein the antibody comprises a VH as in any of the embodiments provided above, and a VL as in any of the embodiments provided above.

In some embodiments, an antibody that binds PD-1 comprising a humanized antibody. In some embodiments, the humanized antibody comprises one or more of the peptides shown in Table 81. In some embodiments, the humanized antibody comprises the amino acid sequence set forth in SEQ ID NO:1009. In some embodiments, the humanized antibody comprises the amino acid sequence set forth in SEQ ID NO:1010.

In some embodiments, the patient is treated with a targeted therapy. In some embodiments, the methods herein include administering a therapeutically effective amount of one or more of 5-fluorouracil (5-FU); capecitabine, irinotecan, oxaliplatin, trifluridine, or tipiracil.

In some embodiments the treatment administered to the subject is determined based upon the treatment output. In some embodiments, if the treatment output indicates that the subject is not likely to respond to pembrolizumab therapy, the treatment recommendation comprises an alternative therapy selected from the group consisting of standard non-checkpoint immunotherapy, standard chemotherapy, combination chemotherapy and non-checkpoint immunotherapy, targeted therapy, radiation therapy, a new generation checkpoint inhibitor alone or in combination, a LAG-3 inhibitor, a recommendation for participation in a clinical trial for an oncotherapeutic, laser therapy, photodynamic therapy, adjuvant treatment with osimertinib for subject with EGFR mutations, targeted therapy for patients with certain gene mutations such as anti-angiogenic agents, drugs that target cells with KRAS gene changes, drugs that target cells with EGFR changes, drugs that target cells with ALK gene changes, drugs that target cells with ROS1 gene changes, drugs that target cells with BRAF gene changes, chemotherapy, cisplatin, carboplatin, paclitaxel, albumin-bound paclitaxel, docetaxel, gemcitabine, vinorelbine, etoposide, pemetrexed, chemotherapy combined with radiation therapy (chemoradiation) and chemoradiation followed by durvalumab

### Machine-Learning Model

In some examples, the methods herein comprise quantifying one or more glycopeptides consisting essentially of an amino acid sequence selected from the group consisting of SEQ ID NO: 1002-1008 using mass spectrometry or tandem liquid chromatography-mass spectrometry. In some examples, the quantification results are used as inputs in a trained model. In some examples, the quantification results are classified or categorized with a predictive algorithm based on the absolute amount, relative amount, and/or type of each glycan or glycopeptide quantified in the test sample, wherein the predictive algorithm is trained on corresponding values for each marker obtained from a population of individuals having known diseases or conditions. In some examples, the disease or condition is cancer. In some cases, the disease or condition is NSCLC.

In some examples, including any of the foregoing, set forth herein is a method for training a machine-learning algorithm, comprising: providing a first data set of MRM transition signals indicative of a sample comprising a glycopeptide consisting essentially of an amino acid sequence selected from the group consisting of SEQ ID NO: 1002-1008; providing a second data set of MRM transition signals indicative of a control sample; and comparing the first data set with the second data set using a machine-learning algorithm.

In some examples, the methods herein include using a sample comprising a glycopeptide consisting of an amino acid sequence selected from the group consisting of SEQ ID NO: 1002-1008 is a sample from a patient having the disease or condition. In some examples, the methods herein include using a sample comprising a glycopeptide consisting essentially of an amino acid sequence selected from the group consisting of SEQ ID NO: 1002-1008 is a sample from a patient having cancer. In some examples, the methods herein include using a control sample, wherein the control sample is a sample from a patient not having the disease or condition.

In some examples, the methods herein include using a sample comprising a glycopeptide consisting essentially of an amino acid sequence selected from the group consisting of SEQ ID NO: 1002-1008, which is a pooled sample from one or more patients having the disease or condition. In some examples, the methods herein include using a control sample, which is a pooled sample from one or more patients not having the disease or condition.

In some examples, the methods include generating machine-learning models trained using mass spectrometry data (e.g., MRM-MS transition signals) from patients having a disease or condition and patients not having a disease or condition. In some examples, the disease or condition is cancer. In some examples, the methods include optimizing the machine-learning models by cross-validation with known standards or other samples. In some examples, the methods include qualifying the performance using the mass spectrometry data to form panels of glycans and glycopeptides with individual sensitivities and specificities. In certain examples, the methods include determining a confidence percent in relation to a diagnosis. In some examples, one to ten glycopeptides consisting essentially of an amino acid sequence selected from the group consisting of SEQ ID NO: 1002-1008 may be useful for diagnosing a patient with the disease or condition with a certain confidence percent. In some examples, ten to fifty glycopeptides consisting essentially of an amino acid sequence selected from the group consisting of SEQ ID NO: 1002-1008 may be useful for diagnosing a patient with the disease or condition with a higher confidence percent.

In some examples, including any of the foregoing, the methods include performing MRM-MS and/or LC-MS on a biological sample. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NO: 1002-1008. In some examples, the methods include comparing, by the computing device, the mass spectra data with the theoretical mass spectra data to generate comparison data indicative of a similarity of each of the plurality of mass spectra to each of the plurality of theoretical target mass spectra associated with a corresponding glycopeptide of the plurality of glycopeptides.

In some examples, machine-learning algorithms are used to determine, by the computing device and based on the MRM-MS data, a distribution of a plurality of characteristic ions in the plurality of mass spectra; and determining, by the computing device and based on the distribution, whether one or more of the plurality of characteristic ions is a glycopeptide ion.

In some examples, the methods herein include training a predictive algorithm. Herein, training the predictive algorithm may refer to supervised learning of a predictive algorithm on the basis of values for one or more glycopeptides consisting of, or consisting essentially of, an amino acid sequence selected from the group consisting of SEQ ID NO: 1002-1008. Training the predictive algorithm may refer to variable selection in a statistical model on the basis of values for one or more glycopeptides consisting essentially of an amino acid sequence selected from the group consisting of SEQ ID NO: 1002-1008. Training a predictive algorithm may for example include determining a weighting vector in feature space for each category, or determining a function or function parameters.

In some examples, the machine-learning algorithm is selected from the group consisting of a deep learning algorithm, a neural network algorithm, an artificial neural network algorithm, a supervised machine-learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof. In certain examples, the machine-learning algorithm is generalized additive model regression.

In some examples, the machine-learning algorithm is generalized additive model, LASSO, Ridge Regression, Random Forests, K-nearest Neighbors (KNN), Deep Neural Networks (DNN), and Principal Components Analysis (PCA). In certain examples, DNN's are used to process mass spec data into analysis-ready forms. In some examples, DNN's are used for peak picking from a mass spectra. In some examples, PCA is useful in feature detection.

In some examples, LASSO is used to provide feature selection. In some examples, elastic net regression is used to provide feature selection.

In some examples, machine-learning algorithms are used to quantify peptides from each protein that are representative of the protein abundance. In some examples, this quantification includes quantifying proteins for which glycosylation is not measured.

In some examples, glycopeptide sequences are identified by fragmentation in the mass spectrometer and database search using Byonic software (Protein Metrics Inc).

In some examples, the methods herein include unsupervised learning to detect features of MRM-MS data that represent known biological quantities, such as protein function or glycan motifs. In certain examples, these features are used as input for classifying by machine-learning. In some examples, the classification is performed using generalized additive models, LASSO, Ridge Regression, or Random Forest nature.

In some examples, the methods herein include mapping input data (e.g., MRM transition peaks) to a value (e.g., a scale based on 0-100) before processing the value in an algorithm. For example, after an MRM transition is identified and the peak characterized, the methods herein include assessing the MS scans in an m/z and retention time window around the peak for a given patient. In some examples, the resulting chromatogram is integrated by a machine-learning algorithm that determines the peak start and stop points, and calculates the area bounded by those points and the intensity (height). The resulting integrated value is the abundance, which then feeds into machine-learning and statistical analyses training and data sets. In some examples, a concentration is calculated from the abundance, which then feeds into machine-learning and statistical analyses training and data sets.

In some examples, machine-learning output, in one instance, is used as machine-learning input in another instance. For example, in addition to the PCA being used for a classification process, the DNN data processing feeds into PCA and other analyses. This results in at least three levels of algorithmic processing. Other hierarchical structures are contemplated within the scope of the instant disclosure.

In some examples, the methods include comparing the amount of each glycan or glycopeptide quantified in the sample to corresponding reference values for each glycan or glycopeptide in a predictive algorithm. In some examples, the methods include a comparative process by which the amount of a glycan or glycopeptide quantified in the sample is compared to a reference value for the same glycan or glycopeptide using a predictive algorithm. The comparative process may be part of a classification by a predictive algorithm. The comparative process may occur at an abstract level, e.g., in n-dimensional feature space or in a higher dimensional space.

In some examples, the methods herein include classifying a patient's sample based on the amount of each glycan or glycopeptide quantified in the sample with a predictive algorithm. In some examples, the methods include using statistical or machine-learning classification processes by which the amount of a glycan or glycopeptide quantified in the test sample is used to determine a category of health with a predictive algorithm. In some examples, the predictive algorithm is a statistical or machine-learning classification algorithm.

In some examples, classification by a predictive algorithm may include scoring likelihood of a panel of glycan or glycopeptide values belonging to each possible category, and determining the highest-scoring category. Classification by a predictive algorithm may include comparing a panel of marker values to previous observations by means of a distance function. Examples of predictive algorithms suitable for classification include generalized additive models, random forests, support vector machines, logistic regression (e.g. multiclass or multinomial logistic regression, and/or algorithms adapted for sparse logistic regression). A wide variety of other predictive algorithms that are suitable for classification may be used, as known to a person skilled in the art.

In some examples, the methods herein include supervised learning of a predictive algorithm on the basis of values for each glycan or glycopeptide obtained from a population of individuals having a disease or condition (e.g., NSCLC). In some examples, the methods include variable selection in a statistical model on the basis of values for each glycan or glycopeptide obtained from a population of individuals having the disease or condition. Training a predictive algorithm may for example include determining a weighting vector in feature space for each category, or determining a function or function parameters.

In one embodiment, the reference value is the amount of a glycan or glycopeptide in a sample or samples derived from one individual. Alternatively, the reference value may be derived by pooling data obtained from multiple individuals, and calculating an average (for example, mean or median) amount for a glycan or glycopeptide. Thus, the reference value may reflect the average amount of a glycan or glycopeptide in multiple individuals. Said amounts may be expressed in absolute or relative terms, in the same manner as described herein.

In some examples, the reference value may be derived from the same sample as the sample that is being tested, thus allowing for an appropriate comparison between the two. For example, if the sample is derived from urine, the reference value is also derived from urine. In some examples, if the sample is a blood sample (e.g. a plasma or a serum sample), then the reference value will also be a blood sample (e.g. a plasma sample or a serum sample, as appropriate). When comparing between the sample and the reference value, the way in which the amounts are expressed is matched between the sample and the reference value. Thus, an absolute amount can be compared with an absolute amount, and a relative amount can be compared with a relative amount. Similarly, the way in which the amounts are expressed for classification with the predictive algorithm is matched to the way in which the amounts are expressed for training the predictive algorithm.

When the amounts of the glycan or glycopeptide are determined, the method may comprise comparing the amount of each glycan or glycopeptide to its corresponding reference value. When the cumulative amount of one, some or all the glycan or glycopeptides are determined, the method may comprise comparing the cumulative amount to a corresponding reference value. When the amounts of the glycan or glycopeptides are combined with each other in a formula to form an index value, the index value can be compared to a corresponding reference index value derived in the same manner.

The reference values may be obtained either within (i.e., constituting a step of) or external to the (i.e., not constituting a step of) methods described herein. In some examples, the methods include a step of establishing a reference value for the quantity of the markers. In other examples, the reference values are obtained externally to the method described herein and accessed during the comparison step of the invention.

In certain embodiments, the generalized additive model may be a regression model or other classification model that may be evaluated utilizing receiver operating characteristic (ROC) evaluation and/or area under curve (AOC) evaluation. For example, in certain embodiments, the ROC model evaluation may represent a plot of sensitivity rate (e.g., patient likely not responsive) against a plot of specificity rate (patient likely to be responsive) and may be further optimized based on an iterative tuning of hyperparameters of the generalized additive model. The trained generalized additive model may be then utilized to predict patient overall survival (OS) for various glycopeptide fragments, in accordance with the presently disclosed embodiments.

In some examples, including any of the foregoing, training of a predictive algorithm may be obtained either within (i.e., constituting a step of) or external to (i.e., not constituting a step of) the methods set forth herein. In some examples, the methods include a step of training of a predictive algorithm. In some examples, the predictive algorithm is trained externally to the method herein and accessed during the classification step of the invention. The reference value may be determined by quantifying the amount of a glycan or glycopeptide in a sample obtained from a population of healthy individual(s). The predictive algorithm may be trained by quantifying the amount of a glycan or glycopeptide in a sample obtained from a population of healthy individual(s). As used herein, the term “healthy individual” refers to an individual or group of individuals who are in a healthy state, e.g., patients who have not shown any symptoms of the disease, have not been diagnosed with the disease and/or are not likely to develop the disease. Preferably said healthy individual(s) is not on medication affecting the disease and has not been diagnosed with any other disease. The one or more healthy individuals may have a similar sex, age and body mass index (BMI) as compared with the test individual. The reference value may be determined by quantifying the amount of a glycan or glycopeptide in a sample obtained from a population of individual(s) suffering from the disease. The predictive algorithm may be trained by quantifying the amount of a marker in a sample obtained from a population of individual(s) suffering from the disease. More preferably such individual(s) may have similar sex, age and body mass index (BMI) as compared with the test individual. The reference value may be obtained from a population of individuals suffering from cancer. The predictive algorithm may be trained by quantifying the amount of a glycan or glycopeptide in a sample obtained from a population of individuals having NSCLC who have been treated with pembrolizumab therapy or combination pembrolizumab and chemotherapy. Once the characteristic glycan or glycopeptide profile of individuals responsive to pembrolizumab therapy is determined, the profile of markers from a biological sample obtained from an individual with NSCLC may be compared to this reference profile to determine whether the test subject will also be responsive to pembrolizumab therapy. Once the predictive algorithm is trained to classify those individuals responsive to pembrolizumab therapy, the profile of markers from a biological sample obtained from an individual diagnosed with NSCLC may be classified by the predictive algorithm to determine whether the test subject is also likely responsive to pembrolizumab therapy.

### Kits

In some examples, including any of the foregoing, set forth herein is a kit comprising a glycopeptide standard, a buffer, and one or more glycopeptides consisting of an amino acid sequence selected from the group consisting of SEQ ID NO: 1002-1008.

In some examples, including any of the foregoing, set forth herein is a kit comprising a glycopeptide standard, a buffer, and one or more glycopeptides consisting essentially of an amino acid sequence selected from the group consisting of SEQ ID NO: 1002-1008.

In some examples, set forth herein is a kit comprising a glycopeptide standard, a buffer, and one or more glycopeptides consisting of an amino acid sequence selected from the group consisting of SEQ ID NO: 1002-1008, and combinations thereof. In some examples, set forth herein is a kit comprising a glycopeptide standard, a buffer, and one or more glycopeptides consisting essentially of an amino acid sequence selected from the group consisting of SEQ ID NO: 1002-1008.

In some examples, set forth herein is a kit for diagnosing or monitoring cancer in an individual wherein the glycan or glycopeptide profile of a sample from said individual is determined and the measured profile is compared with a profile of a normal patient or a profile of a patient with a family history of cancer. In some examples, the kit comprises one or more glycopeptides consisting of an amino acid sequence selected from the group consisting SEQ ID NO: 1002-1008. In some examples, the kit comprises one or more glycopeptides consisting essentially of an amino acid sequence selected from the group consisting of SEQ ID NO: 1002-1008.

In some examples, set forth herein is a kit comprising the reagents for quantification of the oxidized, nitrated, and/or glycated free adducts derived from glycopeptides.

### Compositions

Also provided herein are compositions comprising the glycoproteins, glycopeptides, and peptides provided herein. In some embodiments, provided herein is a composition comprising one or more different peptide structures, two or more different peptide structures, three or more different peptide structures, four or different more peptide structures, five or different more peptide structures, six different peptide structures, or all seven peptide structures comprising the sequences set forth in SEQ ID NO:6-12

### Clinical Assays

In some examples, the biomarkers, methods, and/or kits may be used in a clinical setting for diagnosing patients. In some of these examples, the analysis of samples includes the use of internal standards. These standards may include one or more glycopeptides consisting of an amino acid sequence selected from the group consisting of SEQ ID NO: 1002-1008. These standards may include one or more glycopeptides consisting essentially of an amino acid sequence selected from the group consisting of SEQ ID NO: 1002-1008.

In a clinical setting, samples may be prepared (e.g., by digestion) to include one or more glycopeptides consisting of an amino acid sequence selected from the group consisting of SEQ ID NO: 1002-1008. In a clinical setting, samples may be prepared (e.g., by digestion) to include one or more glycopeptides consisting essentially of an amino acid sequence selected from the group consisting of SEQ ID NO: 1002-1008. In some examples, the amount of a glycan or glycopeptide may be assessed by comparing the amount of one or more glycopeptides consisting of an amino acid sequence selected from the group consisting of SEQ ID NO: 1002-1008 to the concentration of another biomarker. In some examples, the amount of a glycan or glycopeptide may be assessed by comparing the amount of one or more glycopeptides consisting essentially of an amino acid sequence selected from the group consisting of SEQ ID NO: 1002-1008 to the concentration of another biomarker.

In some examples, the amount of a glycan or glycopeptide may be assessed by comparing the amount of one or more glycopeptides consisting of an amino acid sequence selected from the group consisting of SEQ ID NO: 1002-1008 to the amount of one or more glycopeptides consisting of an amino acid sequence selected from the group consisting of SEQ ID NO: 1002-1008.

In some examples, the amount of a glycan or glycopeptide may be assessed by comparing the amount of one or more glycopeptides consisting essentially of an amino acid sequence selected from the group consisting of SEQ ID NO: 1002-1008 to the amount of one or more glycopeptides consisting essentially of an amino acid sequence selected from the group consisting of SEQ ID NO: 1002-1008.

In some examples, including any of the foregoing, the kit may include software for computing the normalization of a glycopeptide MRM transition signal.

In some examples, including any of the foregoing, the kit may include software for quantifying the amount of a glycopeptide consisting of, or consisting essentially of, an amino acid sequence selected from the group consisting of SEQ ID NO: 1002-1008. In some examples, including any of the foregoing, the kit may include software for quantifying the relative amount of a glycopeptide consisting of, or consisting essentially of, an amino acid sequence selected from the group consisting of SEQ ID NO: 1002-1008.

In some examples, including any of the foregoing, a trained model is stored on a server which is accessed by a clinician performing a method, set forth herein. In some examples, the clinician inputs the quantification of the MRM transition signals from a patient's sample into a trained model which are stored on a server. In some examples, the server is accessed by the internet, wireless communication, or other digital or telecommunication methods.

In some examples, including any of the foregoing, a trained model is stored on a server which is accessed by a clinician performing a method, set forth herein. In some examples, the clinician inputs the quantification of the glycopeptide or glycopeptides consisting of, or consisting essentially of, an amino acid sequence selected from the group consisting of SEQ ID NO: 1002-1008 from a patient's sample into a trained model which are stored on a server. In some examples, the server is accessed by the internet, wireless communication, or other digital or telecommunication

Individual KM curves may be plotted for the markers relevant in for the disease interest in four files. Hazard ratios and p-values on the plots are representative of the plotted high/low split at median biomarker expression. FIGS. 147A and 147B show overall survival (OS) Kaplan-Meier curves of patients with non-small-cell lung cancer (NSCLC) for various glycopeptide fragments.

In the descriptions herein, it is understood that every description, variation, embodiment or aspect of a biomarker, peptide, glycopeptide, glycoprotein may be combined with every description, variation, embodiment or aspect of other biomarkers, peptides, glycopeptide, glycoproteins the same as if each and every combination of descriptions is specifically and individually listed.

### II. Additional Considerations

Any headers and/or sub-headers between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### XV. EXAMPLES

The following examples are included to demonstrate particular embodiments of the disclosure. It should be appreciated by those of skill in the art that the techniques disclosed in the examples that follow represent techniques discovered by the inventors to function well in the practice of the embodiments of the disclosure, and thus can be considered to constitute particular modes for its practice. However, those of skill in the art should, in light of the present disclosure, appreciate that many changes can be made in the various embodiments that are disclosed and still obtain a like or similar result without departing from the spirit and scope of the disclosure.

### Example 1

### Serum Glycoprotein Markers for Predicting Stages of Fibrosis in Non-Alcoholic Steatohepatitis

Examples of methods and results are provided that are related to identification of glycoprotein markers in the serum of individuals that allow prediction of different stages of fibrosis in non-alcoholic steatohepatitis (NASH). Serum from 142 healthy controls (76—female, 66—male) were obtained from a commercial provider. Serum from 109 NASH patients (20 biological female, 88 biological male, and 1 biological sex unknown) was obtained from two different commercial providers. NASH fibrosis staging among the individuals with NASH was as follows: F1-38 individuals, F2-17 individuals, F3-42 individuals, F4-4 individuals. Commercially obtained serum reference samples were run alongside patient samples. Samples were processed for analysis using a proprietary method of the Applicant. Processed samples were analyzed on a Triple Quadrupole (QQQ) that was operated in dynamic Multiple reaction monitoring (MRM) mode. The inventors used PB-net, a peak picking software built in-house, to quantify area under the peaks. The python statistical library Scikit-learn was used for all statistical analyses.

In the study, the inventors sought to distinguish serum from NASH patients from control and to distinguish NASH—early (F1&F2) and NASH—late (F3&F4). MRM analysis was performed on control- and NASH-serum. The transition list consisted of glycopeptides and non-glycosylated peptides from proteins that are commonly implicated in various diseases. Prognostic markers were sought with abundance levels significantly different between control and NASH and significantly different between early and late stages of NASH. There were at least 48 glycopeptide and peptide markers that met the aforementioned significance criteria and that also showed the same direction of change in control vs NASH and NASH (early) vs NASH (late). For building the models a 70:30 split was used between train: test set. In a specific embodiment, 14 out these 48 were utilized to train the models. The logistic regression classifier for control vs NASH achieved a test set accuracy of 96% and test AUC of 0.96 (FIG. 12). When a logistic regression binary classifier model was developed of NASH—early (F1&F2) vs NASH —late (F3&F4), a test set accuracy of about 77% and test AUC of 0.83 was achieved (FIG. 11).

### Example 2

### Predicting Breast Cancer in Women Using Liquid Biopsy Derived Glycoproteomic Markers

Examples of methods and results are provided that are related to identification of glycoprotein markers in the peripheral blood of individuals that allow prediction or identification for the presence of breast cancer.

Serum samples from 279 breast cancer patients (median age 56 years) at various stages acquired from Bay Biosciences and iSpecimen and 102 healthy control samples (median age 52 years) acquired from Precision for Medicine. A panel of 596 serum glycosylated and non-glycosylated peptides, representing 71 serum proteins, were analyzed. Age-adjusted differential expression analysis for 596 normalized biomarkers were performed to evaluate statistically significant differential abundances using an FDR-adjusted q-value of 0.05 as a cutoff. Using the top 243 differentially expressed markers as input, a LASSO penalized logistic regression model with 5-fold repeated cross validation was applied to identify the top biomarkers contributing to the separation between healthy controls and breast cancer patients.

Similarly, another model was built with the same steps as above except in this model only stage 1 (n=83) and stage 2 (n=111) samples were included along with 102 healthy control samples to assess the performance of our platform in early-stage breast cancer detection.

### Example 3

### Glycoproteomic Biomarkers in Samples for Pancreatic Cancer Screening

Examples of methods and results are provided that are related to identification of glycoprotein markers in the peripheral blood of individuals that allow prediction or identification for the presence of pancreatic cancer. Peripheral blood from 194 healthy controls were obtained from a commercial provider. In Model 1, peripheral blood from 290 pancreatic cancer patients was obtained from two different commercial providers. In Model 2, peripheral blood from 308 pancreatic cancer patients was obtained from two commercial providers and an academic institution. In Model 3, peripheral blood from 205 early stage pancreatic cancer patients was obtained from two commercial providers and an academic institution. Commercially obtained reference samples were run alongside patient samples, and the reference samples included healthy controls (Model 1), healthy controls and benign pancreatitis patients (Model 2), and healthy controls and benign pancreatitis patients (Model 3). Samples were processed for analysis using a proprietary method of the Applicant. Processed samples were analyzed on a Triple Quadrupole (QQQ) that was operated in dynamic Multiple reaction monitoring (MRM) mode. The R programming language was used to perform statistical analyses and generate visualizations, and the R package ‘caret’ was used for ML modeling procedures.

In the study, the inventors sought to distinguish peripheral blood from individuals from control and individuals with pancreatic cancer. MRM analysis was performed on control- and pancreatic cancer peripheral blood. The transition list comprised glycopeptides and non-glycosylated peptides from proteins that are commonly implicated in various diseases. Prognostic markers were sought with abundance levels significantly different between control and pancreatic cancer and, for Model 3, significantly different between control and early stage pancreatic cancer. There were at least 55 glycopeptide and peptide markers that met the aforementioned significance criteria and that also showed the same direction of change in control vs. pancreatic cancer (or early stage pancreatic cancer). For building the models, a 70:30 split was used between train: test set. In a specific embodiment, 22, 19, or 17 out these 55 were utilized to train the respective Models 1, 2, and 3. For a logistic regression binary classifier model developed of healthy vs. pancreatic cancer patients (Model 1), a test set accuracy of about 0.971 and test AUC of 0.988 was achieved. For a logistic regression binary classifier model developed of healthy+benign vs. pancreatic cancer patients (Model 2), a test set accuracy of about 0.863 and test AUC of 0.892 was achieved. For a logistic regression binary classifier model developed of healthy+benign vs. early-stage pancreatic cancer patients (Model 3), a test set accuracy of about 0.825 and test AUC of 0.873 was achieved.

### Example 4. Sample Preparation and MS Data Production for Training a Diagnostic Model

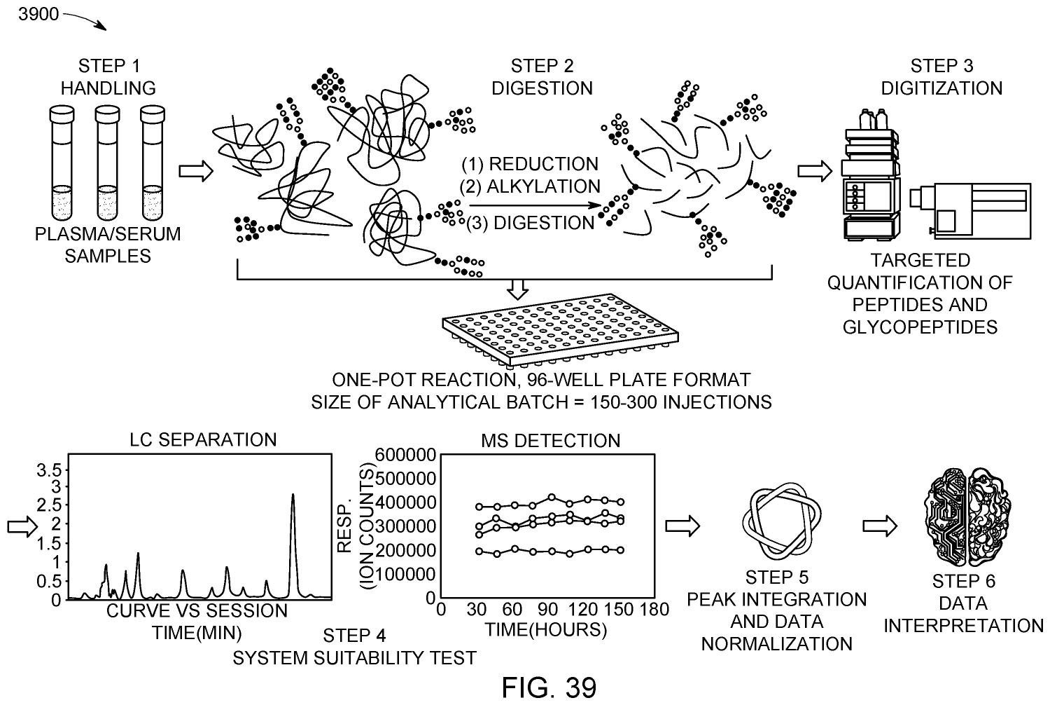

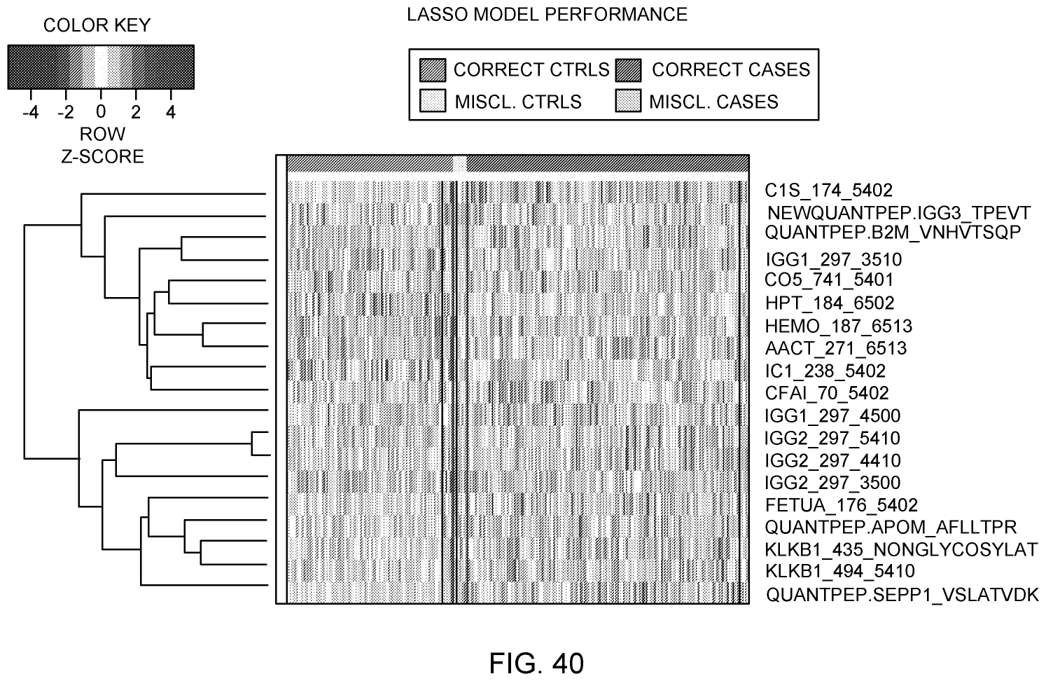

A schematic for the overall workflow for sample preparation and analysis is given in FIG. 39, in which the data interpretation process as illustrated by Step 6 may include performing one or more machine-learning model based prediction and/or classification tasks utilizing a regularized regression model (e.g., LASSO regression model) as described herein. In this workflow, 694 glycopeptide (GP) and non-glycosylated peptide transitions were interrogated, which were derived from 74 serum proteins in pre-treatment peripheral blood samples from a cohort of 194 healthy individuals and a cohort of 316 individuals with NSCLC, as shown in Table 33.

Individuals were assessed prior to sample analysis, using clinical criteria for the NSCLC diagnosis and staging. Lung biopsy samples were subject to a histopathological assessment and clinical imaging techniques were used to stage individuals having NSCLC. The stage of the NSCLC was assigned using the TNM system that is based on the size and extent of the main tumor (T), the spread to nearby lymph nodes (N), and the spread (metastasis) to distant sites (M).

TABLE 33
Summary of patient age and sex

| | No. of | Age | Median | | | Cancer |
|---|---|---|---|---|---|---|
| Status | individuals | range | Age | Female | Male | Stage (0-4) |
| Healthy | 194 | 30-63 | 52 | 102 | 92 | N/A |
| NSCLC | 316 | 31-89 | 66 | 128 | 187 | 1/99/80/84/49 |

*For NSCLC cohort: 1 individual with unknown sex, and 3 individuals with unknown cancer stage.

Pooled human serum for assay normalization and calibration purposes, dithiothreitol (DTT), and iodoacetamide (IAA) were purchased from Millipore Sigma (St. Louis, MO). Sequencing grade trypsin was purchased from Promega (Madison, WI). Acetonitrile (LC-MS grade) was purchased from Honeywell (Muskegon, MI). All other reagents used were procured from Millipore Sigma, VWR, and Fisher Scientific.

Prior to analysis, plasma samples were reduced with DTT in a water bath at 60° C. for 50 min, then alkylated with IAA followed by digestion with trypsin in a water bath at 37° C. for 18 hours. To quench the digestion, formic acid was added to each sample after incubation to a final concentration of 1% (v/v). A pool of 18 stable isotope-labeled synthetic peptides matching the sequence of 18 endogenous peptide targets were included at a known concentration for the purpose of determining absolute endogenous protein concentrations in the samples.

Digested plasma samples were injected into an Agilent 6495B triple quadrupole mass spectrometer equipped with an Agilent 1290 Infinity ultra-high-pressure (UHP)—LC system and an Agilent ZORBAX Eclipse Plus C18 column (2.1 mm×150 mm i.d., 1.8 μm particle size). Separation of the peptides and glycopeptides was performed using a 49-min binary gradient. The aqueous mobile phase A was 3% acetonitrile, 0.1% formic acid in water (v/v), and the organic mobile phase B was 90% acetonitrile, 0.1% formic acid in water (v/v). The flow rate was set at 0.5 mL/min. Electrospray ionization (ESI) was used as the ionization source and was operated in positive ion mode. The triple quadrupole MS was operated in dynamic multiple reaction monitoring (dMRM) mode. Samples were injected in a randomized fashion with regard to underlying phenotype, and reference pooled serum digests were injected interspersed with study samples, at every 10^{th} sample position throughout the run. The R library ‘caret’ was used for building machine-learning models, and all other statistical analyses used base R functions.

Concentration of peptides and glycopeptides were calculated by the following formula:

$$[ peptide ] = \frac{peptide raw abundance}{ISTD raw abundance} \times [ ISTD ] \times dilution factor$$

Wherein the internal standard (ISTD) corresponds to the peptide of interest, optionally as a stable isotope internal standard (e.g., heavy labeled lysine). An example ISTD could be GWVTDGFSSLK* where the terminal lysine is a heavy stable isotope labeled lysine for the unlabeled peptide with sequence—GWVTDGFSSLK.

$$glycopeptide site occupancy = \frac{glycopeptide raw abundance}{glycoprotein raw abundance}$$

Wherein the glycoprotein raw abundance is the raw abundance sum of all of the glycopeptides from the same glycoprotein.

$${[ glycopeptide ]}_{approximate} = glycopeptide site occupancy \times {[ peptide ]}_{glycopeptide}$$

Wherein the [peptide] is the peptide concentration of the quantification peptide from the same glycoprotein.

### Example 5. Analysis of Peptide Structure Data

A MRM-MS analysis was performed on pre-treatment peripheral blood samples from healthy individuals and individuals having NSCLC, wherein digested and processed samples containing one or more of peptide and glycopeptide were subjected to LC-MS/MS running in a MRM mode to quantify the presence, amount, and/or structure of the one or more peptides and glycopeptide. Concentrations of glycopeptides and peptides were calculated as described in Example 4. Provided in Table 35 are the identified peptide sequences and glycan moieties that were determined to have significantly different concentrations for peptides and glycopeptides when comparing the healthy cohort vs the NSCLC cohort. In Tables 36A and 36B, the glycan structures referenced in Table 35 are shown. The proteins and glycoproteins associated with these peptides and glycopeptides, respectively, are summarized in Table 34. In Table 37, the MRM-MS and liquid chromatography (LC) parameters for the peptide and glycopeptide identification are summarized.

TABLE 34
Proteins and glycoproteins associated with NSCLC control, all stages of
NSCLC, early-stage NSCLC, and late-stage NSCLC

| SEQ | Protein | | | |
|---|---|---|---|---|
| ID | Abbre- | | UniProt | |
| NO | viation | Protein Name | ID | Protein Sequence |
| SEQ | A2GL | Leucine-rich | P02750 | MSSWSRQRPKSPGGIQPHVSRTLFLLLLLAASAWGVT |
| ID | | alpha-2- | | LSPKDCQVFRSDHGSSISCQPPAEIPGYLPADTVHLAV |
| NO: | | glycoprotein | | EFFNLTHLPANLLQGASKLQELHLSSNGLESLSPEFLR |
| 197 | | | | PVPQLRVLDLTRNALTGLPPGLFQASATLDTLVLKEN |
| | | | | QLEVLEVSWLHGLKALGHLDLSGNRLRKLPPGLLAN |
| | | | | FTLLRTLDLGENQLETLPPDLLRGPLQLERLHLEGNKL |
| | | | | QVLGKDLLLPQPDLRYLFLNGNKLARVAAGAFQGLR |
| | | | | QLDMLDLSNNSLASVPEGLWASLGQPNWDMRDGFDI |
| | | | | SGNPWICDQNLSDLYRWLQAQKDKMFSQNDTRCAG |
| | | | | PEAVKGQTLLAVAKSQ |
| SEQ | A2MG | Alpha-2- | P01023 | MGKNKLLHPSLVLLLLVLLPTDASVSGKPQYMVLVPS |
| ID | | macroglobulin | | LLHTETTEKGCVLLSYLNETVTVSASLESVRGNRSLFT |
| NO: | | | | DLEAENDVLHCVAFAVPKSSSNEEVMFLTVQVKGPT |
| 198 | | | | QEFKKRTTVMVKNEDSLVFVQTDKSIYKPGQTVKFR |
| | | | | VVSMDENFHPLNELIPLVYIQDPKGNRIAQWQSFQLE |
| | | | | GGLKQFSFPLSSEPFQGSYKVVVQKKSGGRTEHPFTV |
| | | | | EEFVLPKFEVQVTVPKIITILEEEMNVSVCGLYTYGKP |
| | | | | VPGHVTVSICRKYSDASDCHGEDSQAFCEKFSGQLNS |
| | | | | HGCFYQQVKTKVFQLKRKEYEMKLHTEAQIQEEGTV |
| | | | | VELTGRQSSEITRTITKLSFVKVDSHFRQGIPFFGQVRL |
| | | | | VDGKGVPIPNKVIFIRGNEANYYSNATTDEHGLVQFSI |
| | | | | NTTNVMGTSLTVRVNYKDRSPCYGYQWVSEEHEEA |
| | | | | HHTAYLVFSPSKSFVHLEPMSHELPCGHTQTVQAHYI |
| | | | | LNGGTLLGLKKLSFYYLIMAKGGIVRTGTHGLLVKQE |
| | | | | DMKGHFSISIPVKSDIAPVARLLIYAVLPTGDVIGDSA |
| | | | | KYDVENCLANKVDLSFSPSQSLPASHAHLRVTAAPQS |
| | | | | VCALRAVDQSVLLMKPDAELSASSVYNLLPEKDLTGF |
| | | | | PGPLNDQDNEDCINRHNVYINGITYTPVSSTNEKDMY |
| | | | | SFLEDMGLKAFTNSKIRKPKMCPQLQQYEMHGPEGL |
| | | | | RVGFYESDVMGRGHARLVHVEEPHTETVRKYFPETW |
| | | | | IWDLVVVNSAGVAEVGVTVPDTITEWKAGAFCLSED |
| | | | | AGLGISSTASLRAFQPFFVELTMPYSVIRGEAFTLKAT |
| | | | | VLNYLPKCIRVSVQLEASPAFLAVPVEKEQAPHCICAN |
| | | | | GRQTVSWAVTPKSLGNVNFTVSAEALESQELCGTEVP |
| | | | | SVPEHGRKDTVIKPLLVEPEGLEKETTFNSLLCPSGGE |
| | | | | VSEELSLKLPPNVVEESARASVSVLGDILGSAMQNTQ |
| | | | | NLLQMPYGCGEQNMVLFAPNIYVLDYLNETQQLTPEI |
| | | | | KSKAIGYLNTGYQRQLNYKHYDGSYSTFGERYGRNQ |
| | | | | GNTWLTAFVLKTFAQARAYIFIDEAHITQALIWLSQR |
| | | | | QKDNGCFRSSGSLLNNAIKGGVEDEVTLSAYITIALLE |
| | | | | IPLTVTHPVVRNALFCLESAWKTAQEGDHGSHVYTK |
| | | | | ALLAYAFALAGNQDKRKEVLKSLNEEAVKKDNSVH |
| | | | | WERPQKPKAPVGHFYEPQAPSAEVEMTSYVLLAYLT |
| | | | | AQPAPTSEDLTSATNIVKWITKQQNAQGGFSSTQDTV |
| | | | | VALHALSKYGAATFTRTGKAAQVTIQSSGTFSSKFQV |
| | | | | DNNNRLLLQQVSLPELPGEYSMKVTGEGCVYLQTSL |
| | | | | KYNILPEKEEFPFALGVQTLPQTCDEPKAHTSFQISLSV |
| | | | | SYTGSRSASNMAIVDVKMVSGFIPLKPTVKMLERSNH |
| | | | | VSRTEVSSNHVLIYLDKVSNQTLSLFFTVLQDVPVRDL |
| | | | | KPAIVKVYDYYETDEFAIAEYNAPCSKDLGNA |
| SEQ | AACT | Alpha-1- | P01011 | MERMLPLLALGLLAAGFCPAVLCHPNSPLDEENLTQE |
| ID | | antichymotrypsin | | NQDRGTHVDLGLASANVDFAFSLYKQLVLKAPDKNV |
| NO: | | | | IFSPLSISTALAFLSLGAHNTTLTEILKGLKFNLTETSEA |
| 199 | | | | EIHQSFQHLLRTLNQSSDELQLSMGNAMFVKEQLSLL |
| | | | | DRFTEDAKRLYGSEAFATDFQDSAAAKKLINDYVKN |
| | | | | GTRGKITDLIKDLDSQTMMVLVNYIFFKAKWEMPFDP |
| | | | | QDTHQSRFYLSKKKWVMVPMMSLHHLTIPYFRDEEL |
| | | | | SCTVVELKYTGNASALFILPDQDKMEEVEAMLLPETL |
| | | | | KRWRDSLEFREIGELYLPKFSISRDYNLNDILLQLGIEE |
| | | | | AFTSKADLSGITGARNLAVSQVVHKAVLDVFEEGTEA |
| | | | | SAATAVKITLLSALVETRTIVRFNRPFLMIIVPTDTQNI |
| | | | | FFMSKVTNPKQA |
| SEQ | AGP1 | Alpha-1-acid | P02763, | MALSWVLTVLSLLPLLEAQIPLCANLVPVPITNATLDR |
| ID | | glycoprotein 1 | | ITGKWFYIASAFRNEEYNKSVQEIQATFFYFTPNKTED |
| NO: | | | | TIFLREYQTRQDQCIYNTTYLNVQRENGTISRYVGGQ |
| 200 | | | | EHFAHLLILRDTKTYMLAFDVNDEKNWGLSVYADKP |
| | | | | ETTKEQLGEFYEALDCLRIPKSDVVYTDWKKDKCEPL |
| | | | | EKQHEKERKQEEGES |
| SEQ | AGP2 | Alpha-1-acid | P19652 | MALSWVLTVLSLLPLLEAQIPLCANLVPVPITNATLDR |
| ID | | glycoprotein 2 | | ITGKWFYIASAFRNEEYNKSVQEIQATFFYFTPNKTED |
| NO: | | | | TIFLREYQTRQNQCFYNSSYLNVQRENGTVSRYEGGR |
| 201 | | | | EHVAHLLFLRDTKTLMFGSYLDDEKNWGLSFYADKP |
| | | | | ETTKEQLGEFYEALDCLCIPRSDVMYTDWKKDKCEPL |
| | | | | EKQHEKERKQEEGES |
| SEQ | APOB | Apolipoprotein | P04114 | MDPPRPALLALLALPALLLLLLAGARAEEEMLENVSL |
| ID | | B-100 | | VCPKDATRFKHLRKYTYNYEAESSSGVPGTADSRSAT |
| NO: | | | | RINCKVELEVPQLCSFILKTSQCTLKEVYGFNPEGKAL |
| 202 | | | | LKKTKNSEEFAAAMSRYELKLAIPEGKQVFLYPEKDE |
| | | | | PTYILNIKRGIISALLVPPETEEAKQVLFLDTVYGNCST |
| | | | | HFTVKTRKGNVATEISTERDLGQCDRFKPIRTGISPLA |
| | | | | LIKGMTRPLSTLISSSQSCQYTLDAKRKHVAEAICKEQ |
| | | | | HLFLPFSYKNKYGMVAQVTQTLKLEDTPKINSRFFGE |
| | | | | GTKKMGLAFESTKSTSPPKQAEAVLKTLQELKKLTISE |
| | | | | QNIQRANLFNKLVTELRGLSDEAVTSLLPQLIEVSSPIT |
| | | | | LQALVQCGQPQCSTHILQWLKRVHANPLLIDVVTYLV |
| | | | | ALIPEPSAQQLREIFNMARDQRSRATLYALSHAVNNY |
| | | | | HKTNPTGTQELLDIANYLMEQIQDDCTGDEDYTYLIL |
| | | | | RVIGNMGQTMEQLTPELKSSILKCVQSTKPSLMIQKA |
| | | | | AIQALRKMEPKDKDQEVLLQTFLDDASPGDKRLAAY |
| | | | | LMLMRSPSQADINKIVQILPWEQNEQVKNFVASHIANI |
| | | | | LNSEELDIQDLKKLVKEALKESQLPTVMDFRKFSRNY |
| | | | | QLYKSVSLPSLDPASAKIEGNLIFDPNNYLPKESMLKT |
| | | | | TLTAFGFASADLIEIGLEGKGFEPTLEALFGKQGFFPDS |
| | | | | VNKALYWVNGQVPDGVSKVLVDHFGYTKDDKHEQ |
| | | | | DMVNGIMLSVEKLIKDLKSKEVPEARAYLRILGEELG |
| | | | | FASLHDLQLLGKLLLMGARTLQGIPQMIGEVIRKGSK |
| | | | | NDFFLHYIFMENAFELPTGAGLQLQISSSGVIAPGAKA |
| | | | | GVKLEVANMQAELVAKPSVSVEFVTNMGIIIPDFARS |
| | | | | GVQMNTNFFHESGLEAHVALKAGKLKFIIPSPKRPVK |
| | | | | LLSGGNTLHLVSTTKTEVIPPLIENRQSWSVCKQVFPG |
| | | | | LNYCTSGAYSNASSTDSASYYPLTGDTRLELELRPTGE |
| | | | | IEQYSVSATYELQREDRALVDTLKFVTQAEGAKQTEA |
| | | | | TMTFKYNRQSMTLSSEVQIPDFDVDLGTILRVNDESTE |
| | | | | GKTSYRLTLDIQNKKITEVALMGHLSCDTKEERKIKG |
| | | | | VISIPRLQAEARSEILAHWSPAKLLLQMDSSATAYGST |
| | | | | VSKRVAWHYDEEKIEFEWNTGTNVDTKKMTSNFPVD |
| | | | | LSDYPKSLHMYANRLLDHRVPQTDMTFRHVGSKLIV |
| | | | | AMSSWLQKASGSLPYTQTLQDHLNSLKEFNLQNMGL |
| | | | | PDFHIPENLFLKSDGRVKYTLNKNSLKIEIPLPFGGKSS |
| | | | | RDLKMLETVRTPALHFKSVGFHLPSREFQVPTFTIPKL |
| | | | | YQLQVPLLGVLDLSTNVYSNLYNWSASYSGGNTSTD |
| | | | | HFSLRARYHMKADSVVDLLSYNVQGSGETTYDHKNT |
| | | | | FTLSYDGSLRHKFLDSNIKFSHVEKLGNNPVSKGLLIF |
| | | | | DASSSWGPQMSASVHLDSKKKQHLFVKEVKIDGQFR |
| | | | | VSSFYAKGTYGLSCQRDPNTGRLNGESNLRFNSSYLQ |
| | | | | GTNQITGRYEDGTLSLTSTSDLQSGIIKNTASLKYENY |
| | | | | ELTLKSDTNGKYKNFATSNKMDMTFSKQNALLRSEY |
| | | | | QADYESLRFFSLLSGSLNSHGLELNADILGTDKINSGA |
| | | | | HKATLRIGQDGISTSATTNLKCSLLVLENELNAELGLS |
| | | | | GASMKLTTNGRFREHNAKFSLDGKAALTELSLGSAY |
| | | | | QAMILGVDSKNIFNFKVSQEGLKLSNDMMGSYAEMK |
| | | | | FDHTNSLNIAGLSLDFSSKLDNIYSSDKFYKQTVNLQL |
| | | | | QPYSLVTTLNSDLKYNALDLTNNGKLRLEPLKLHVA |
| | | | | GNLKGAYQNNEIKHIYAISSAALSASYKADTVAKVQG |
| | | | | VEFSHRLNTDIAGLASAIDMSTNYNSDSLHFSNVFRSV |
| | | | | MAPFTMTIDAHTNGNGKLALWGEHTGQLYSKFLLKA |
| | | | | EPLAFTFSHDYKGSTSHHLVSRKSISAALEHKVSALLT |
| | | | | PAEQTGTWKLKTQFNNNEYSQDLDAYNTKDKIGVEL |
| | | | | TGRTLADLTLLDSPIKVPLLLSEPINIIDALEMRDAVEK |
| | | | | PQEFTIVAFVKYDKNQDVHSINLPFFETLQEYFERNRQ |
| | | | | TIIVVLENVQRNLKHINIDQFVRKYRAALGKLPQQAN |
| | | | | DYLNSFNWERQVSHAKEKLTALTKKYRITENDIQIAL |
| | | | | DDAKINFNEKLSQLQTYMIQFDQYIKDSYDLHDLKIAI |
| | | | | ANIIDEIIEKLKSLDEHYHIRVNLVKTIHDLHLFIENIDF |
| | | | | NKSGSSTASWIQNVDTKYQIRIQIQEKLQQLKRHIQNI |
| | | | | DIQHLAGKLKQHIEAIDVRVLLDQLGTTISFERINDILE |
| | | | | HVKHFVINLIGDFEVAEKINAFRAKVHELIERYEVDQQ |
| | | | | IQVLMDKLVELAHQYKLKETIQKLSNVLQQVKIKDYF |
| | | | | EKLVGFIDDAVKKLNELSFKTFIEDVNKFLDMLIKKLK |
| | | | | SFDYHQFVDETNDKIREVTQRLNGEIQALELPQKAEA |
| | | | | LKLFLEETKATVAVYLESLQDTKITLIINWLQEALSSA |
| | | | | SLAHMKAKFRETLEDTRDRMYQMDIQQELQRYLSLV |
| | | | | GQVYSTLVTYISDWWTLAAKNLTDFAEQYSIQDWAK |
| | | | | RMKALVEQGFTVPEIKTILGTMPAFEVSLQALQKATF |
| | | | | QTPDFIVPLTDLRIPSVQINFKDLKNIKIPSRFSTPEFTIL |
| | | | | NTFHIPSFTIDFVEMKVKIIRTIDQMLNSELQWPVPDIY |
| | | | | LRDLKVEDIPLARITLPDFRLPEIAIPEFIIPTLNLNDFQV |
| | | | | PDLHIPEFQLPHISHTIEVPTFGKLYSILKIQSPLFTLDA |
| | | | | NADIGNGTTSANEAGIAASITAKGESKLEVLNFDFQA |
| | | | | NAQLSNPKINPLALKESVKFSSKYLRTEHGSEMLFFGN |
| | | | | AIEGKSNTVASLHTEKNTLELSNGVIVKINNQLTLDSN |
| | | | | TKYFHKLNIPKLDFSSQADLRNEIKTLLKAGHIAWTSS |
| | | | | GKGSWKWACPRFSDEGTHESQISFTIEGPLTSFGLSNK |
| | | | | INSKHLRVNQNLVYESGSLNFSKLEIQSQVDSQHVGH |
| | | | | SVLTAKGMALFGEGKAEFTGRHDAHLNGKVIGTLKN |
| | | | | SLFFSAQPFEITASTNNEGNLKVRFPLRLTGKIDFLNNY |
| | | | | ALFLSPSAQQASWQVSARFNQYKYNQNFSAGNNENI |
| | | | | MEAHVGINGEANLDFLNIPLTIPEMRLPYTIITTPPLKD |
| | | | | FSLWEKTGLKEFLKTTKQSFDLSVKAQYKKNKHRHSI |
| | | | | TNPLAVLCEFISQSIKSFDRHFEKNRNNALDFVTKSYN |
| | | | | ETKIKFDKYKAEKSHDELPRTFQIPGYTVPVVNVEVSP |
| | | | | FTIEMSAFGYVFPKAVSMPSFSILGSDVRVPSYTLILPS |
| | | | | LELPVLHVPRNLKLSLPDFKELCTISHIFIPAMGNITYD |
| | | | | FSFKSSVITLNTNAELFNQSDIVAHLLSSSSSVIDALQY |
| | | | | KLEGTTRLTRKRGLKLATALSLSNKFVEGSHNSTVSL |
| | | | | TTKNMEVSVATTTKAQIPILRMNFKQELNGNTKSKPT |
| | | | | VSSSMEFKYDFNSSMLYSTAKGAVDHKLSLESLTSYF |
| | | | | SIESSTKGDVKGSVLSREYSGTIASEANTYLNSKSTRSS |
| | | | | VKLQGTSKIDDIWNLEVKENFAGEATLQRIYSLWEHS |
| | | | | TKNHLQLEGLFFTNGEHTSKATLELSPWQMSALVQV |
| | | | | HASQPSSFHDFPDLGQEVALNANTKNQKIRWKNEVRI |
| | | | | HSGSFQSQVELSNDQEKAHLDIAGSLEGHLRFLKNIIL |
| | | | | PVYDKSLWDFLKLDVTTSIGRRQHLRVSTAFVYTKNP |
| | | | | NGYSFSIPVKVLADKFIIPGLKLNDLNSVLVMPTFHVP |
| | | | | FTDLQVPSCKLDFREIQIYKKLRTSSFALNLPTLPEVKF |
| | | | | PEVDVLTKYSQPEDSLIPFFEITVPESQLTVSQFTLPKS |
| | | | | VSDGIAALDLNAVANKIADFELPTIIVPEQTIEIPSIKFS |
| | | | | VPAGIVIPSFQALTARFEVDSPVYNATWSASLKNKAD |
| | | | | YVETVLDSTCSSTVQFLEYELNVLGTHKIEDGTLASKT |
| | | | | KGTFAHRDFSAEYEEDGKYEGLQEWEGKAHLNIKSP |
| | | | | AFTDLHLRYQKDKKGISTSAASPAVGTVGMDMDEDD |
| | | | | DFSKWNFYYSPQSSPDKKLTIFKTELRVRESDEETQIK |
| | | | | VNWEEEAASGLLTSLKDNVPKATGVLYDYVNKYHW |
| | | | | EHTGLTLREVSSKLRRNLQNNAEWVYQGAIRQIDDID |
| | | | | VRFQKAASGTTGTYQEWKDKAQNLYQELLTQEGQA |
| | | | | SFQGLKDNVFDGLVRVTQEFHMKVKHLIDSLIDFLNF |
| | | | | PRFQFPGKPGIYTREELCTMFIREVGTVLSQVYSKVHN |
| | | | | GSEILFSYFQDLVITLPFELRKHKLIDVISMYRELLKDL |
| | | | | SKEAQEVFKAIQSLKTTEVLRNLQDLLQFIFQLIEDNIK |
| | | | | QLKEMKFTYLINYIQDEINTIFSDYIPYVFKLLKENLCL |
| | | | | NLHKFNEFIQNELQEASQELQQIHQYIMALREEYFDPS |
| | | | | IVGWTVKYYELEEKIVSLIKNLLVALKDFHSEYIVSAS |
| | | | | NFTSQLSSQVEQFLHRNIQEYLSILTDPDGKGKEKIAE |
| | | | | LSATAQEIIKSQAIATKKIISDYHQQFRYKLQDFSDQLS |
| | | | | DYYEKFIAESKRLIDLSIQNYHTFLIYITELLKKLQSTT |
| | | | | VMNPYMKLAPGELTIIL |
| SEQ | APOM | Apolipoprotein | O95445 | MFHQIWAALLYFYGIILNSIYQCPEHSQLTTLGVDGKE |
| ID | | M | | FPEVHLGQWYFIAGAAPTKEELATFDPVDNIVFNMAA |
| NO: | | | | GSAPMQLHLRATIRMKDGLCVPRKWIYHLTEGSTDL |
| 203 | | | | RTEGRPDMKTELFSSSCPGGIMLNETGQGYQRFLLYN |
| | | | | RSPHPPEKCVEEFKSLTSCLDSKAFLLTPRNQEACELS |
| | | | | NN |
| SEQ | B2M | Beta-2- | P61769 | MSRSVALAVLALLSLSGLEAIQRTPKIQVYSRHPAENG |
| ID | | microglobulin | | KSNFLNCYVSGFHPSDIEVDLLKNGERIEKVEHSDLSF |
| NO: | | | | SKDWSFYLLYYTEFTPTEKDEYACRVNHVTLSQPKIV |
| 204 | | | | KWDRDM |
| SEQ | C1S | Complement | P09871 | MWCIVLFSLLAWVYAEPTMYGEILSPNYPQAYPSEVE |
| ID | | C1s | | KSWDIEVPEGYGIHLYFTHLDIELSENCAYDSVQIISGD |
| NO: | | subcomponent | | TEEGRLCGQRSSNNPHSPIVEEFQVPYNKLQVIFKSDF |
| 205 | | | | SNEERFTGFAAYYVATDINECTDFVDVPCSHFCNNFIG |
| | | | | GYFCSCPPEYFLHDDMKNCGVNCSGDVFTALIGEIAS |
| | | | | PNYPKPYPENSRCEYQIRLEKGFQVVVTLRREDFDVE |
| | | | | AADSAGNCLDSLVFVAGDRQFGPYCGHGFPGPLNIET |
| | | | | KSNALDIIFQTDLTGQKKGWKLRYHGDPMPCPKEDTP |
| | | | | NSVWEPAKAKYVFRDVVQITCLDGFEVVEGRVGATS |
| | | | | FYSTCQSNGKWSNSKLKCQPVDCGIPESIENGKVEDP |
| | | | | ESTLFGSVIRYTCEEPYYYMENGGGGEYHCAGNGSW |
| | | | | VNEVLGPELPKCVPVCGVPREPFEEKQRIIGGSDADIK |
| | | | | NFPWQVFFDNPWAGGALINEYWVLTAAHVVEGNRE |
| | | | | PTMYVGSTSVQTSRLAKSKMLTPEHVFIHPGWKLLEV |
| | | | | PEGRTNFDNDIALVRLKDPVKMGPTVSPICLPGTSSDY |
| | | | | NLMDGDLGLISGWGRTEKRDRAVRLKAARLPVAPLR |
| | | | | KCKEVKVEKPTADAEAYVFTPNMICAGGEKGMDSCK |
| | | | | GDSGGAFAVQDPNDKTKFYAAGLVSWGPQCGTYGL |
| | | | | YTRVKNYVDWIMKTMQENSTPRED |
| SEQ | CERU | Ceruloplasmin | P00450 | MKILILGIFLFLCSTPAWAKEKHYYIGIIETTWDYASD |
| ID | | | | HGEKKLISVDTEHSNIYLQNGPDRIGRLYKKALYLQY |
| NO: | | | | TDETFRTTIEKPVWLGFLGPIIKAETGDKVYVHLKNLA |
| 206 | | | | SRPYTFHSHGITYYKEHEGAIYPDNTTDFQRADDKVY |
| | | | | PGEQYTYMLLATEEQSPGEGDGNCVTRIYHSHIDAPK |
| | | | | DIASGLIGPLIICKKDSLDKEKEKHIDREFVVMFSVVDE |
| | | | | NFSWYLEDNIKTYCSEPEKVDKDNEDFQESNRMYSV |
| | | | | NGYTFGSLPGLSMCAEDRVKWYLFGMGNEVDVHAA |
| | | | | FFHGQALTNKNYRIDTINLFPATLFDAYMVAQNPGEW |
| | | | | MLSCQNLNHLKAGLQAFFQVQECNKSSSKDNIRGKH |
| | | | | VRHYYIAAEEIIWNYAPSGIDIFTKENLTAPGSDSAVFF |
| | | | | EQGTTRIGGSYKKLVYREYTDASFTNRKERGPEEEHL |
| | | | | GILGPVIWAEVGDTIRVTFHNKGAYPLSIEPIGVRFNK |
| | | | | NNEGTYYSPNYNPQSRSVPPSASHVAPTETFTYEWTV |
| | | | | PKEVGPTNADPVCLAKMYYSAVDPTKDIFTGLIGPMK |
| | | | | ICKKGSLHANGRQKDVDKEFYLFPTVFDENESLLLED |
| | | | | NIRMFTTAPDQVDKEDEDFQESNKMHSMNGFMYGN |
| | | | | QPGLTMCKGDSVVWYLFSAGNEADVHGIYFSGNTYL |
| | | | | WRGERRDTANLFPQTSLTLHMWPDTEGTFNVECLTT |
| | | | | DHYTGGMKQKYTVNQCRRQSEDSTFYLGERTYYIAA |
| | | | | VEVEWDYSPQREWEKELHHLQEQNVSNAFLDKGEFY |
| | | | | IGSKYKKVVYRQYTDSTFRVPVERKAEEEHLGILGPQ |
| | | | | LHADVGDKVKIIFKNMATRPYSIHAHGVQTESSTVTP |
| | | | | TLPGETLTYVWKIPERSGAGTEDSACIPWAYYSTVDQ |
| | | | | VKDLYSGLIGPLIVCRRPYLKVFNPRRKLEFALLFLVF |
| | | | | DENESWYLDDNIKTYSDHPEKVNKDDEEFIESNKMH |
| | | | | AINGRMFGNLQGLTMHVGDEVNWYLMGMGNEIDLH |
| | | | | TVHFHGHSFQYKHRGVYSSDVFDIFPGTYQTLEMFPR |
| | | | | TPGIWLLHCHVTDHIHAGMETTYTVLQNEDTKSG |
| SEQ | CFAI | Complement | P05156 | MKLLHVFLLFLCFHLRFCKVTYTSQEDLVEKKCLAKK |
| ID | | Factor I | | YTHLSCDKVFCQPWQRCIEGTCVCKLPYQCPKNGTA |
| NO: | | | | VCATNRRSFPTYCQQKSLECLHPGTKFLNNGTCTAEG |
| 207 | | | | KFSVSLKHGNTDSEGIVEVKLVDQDKTMFICKSSWSM |
| | | | | REANVACLDLGFQQGADTQRRFKLSDLSINSTECLHV |
| | | | | HCRGLETSLAECTFTKRRTMGYQDFADVVCYTQKAD |
| | | | | SPMDDFFQCVNGKYISQMKACDGINDCGDQSDELCC |
| | | | | KACQGKGFHCKSGVCIPSQYQCNGEVDCITGEDEVGC |
| | | | | AGFASVTQEETEILTADMDAERRRIKSLLPKLSCGVK |
| | | | | NRMHIRRKRIVGGKRAQLGDLPWQVAIKDASGITCG |
| | | | | GIYIGGCWILTAAHCLRASKTHRYQIWTTVVDWIHPD |
| | | | | LKRIVIEYVDRIIFHENYNAGTYQNDIALIEMKKDGNK |
| | | | | KDCELPRSIPACVPWSPYLFQPNDTCIVSGWGREKDN |
| | | | | ERVFSLQWGEVKLISNCSKFYGNRFYEKEMECAGTY |
| | | | | DGSIDACKGDSGGPLVCMDANNVTYVWGVVSWGEN |
| | | | | CGKPEFPGVYTKVANYFDWISYHVGRPFISQYNV |
| SEQ | CO5 | Complement | P01031 | MGLLGILCFLIFLGKTWGQEQTYVISAPKIFRVGASENI |
| ID | | C5 | | VIQVYGYTEAFDATISIKSYPDKKFSYSSGHVHLSSEN |
| NO: | | | | KFQNSAILTIQPKQLPGGQNPVSYVYLEVVSKHFSKSK |
| 208 | | | | RMPITYDNGFLFIHTDKPVYTPDQSVKVRVYSLNDDL |
| | | | | KPAKRETVLTFIDPEGSEVDMVEEIDHIGIISFPDFKIPS |
| | | | | NPRYGMWTIKAKYKEDFSTTGTAYFEVKEYVLPHFS |
| | | | | VSIEPEYNFIGYKNFKNFEITIKARYFYNKVVTEADVYI |
| | | | | TFGIREDLKDDQKEMMQTAMQNTMLINGIAQVTFDS |
| | | | | ETAVKELSYYSLEDLNNKYLYIAVTVIESTGGFSEEAE |
| | | | | IPGIKYVLSPYKLNLVATPLFLKPGIPYPIKVQVKDSLD |
| | | | | QLVGGVPVTLNAQTIDVNQETSDLDPSKSVTRVDDG |
| | | | | VASFVLNLPSGVTVLEFNVKTDAPDLPEENQAREGYR |
| | | | | AIAYSSLSQSYLYIDWTDNHKALLVGEHLNIIVTPKSP |
| | | | | YIDKITHYNYLILSKGKIIHFGTREKFSDASYQSINIPVT |
| | | | | QNMVPSSRLLVYYIVTGEQTAELVSDSVWLNIEEKCG |
| | | | | NQLQVHLSPDADAYSPGQTVSLNMATGMDSWVALA |
| | | | | AVDSAVYGVQRGAKKPLERVFQFLEKSDLGCGAGGG |
| | | | | LNNANVFHLAGLTFLTNANADDSQENDEPCKEILRPR |
| | | | | RTLQKKIEEIAAKYKHSVVKKCCYDGACVNNDETCE |
| | | | | QRAARISLGPRCIKAFTECCVVASQLRANISHKDMQL |
| | | | | GRLHMKTLLPVSKPEIRSYFPESWLWEVHLVPRRKQL |
| | | | | QFALPDSLTTWEIQGVGISNTGICVADTVKAKVFKDV |
| | | | | FLEMNIPYSVVRGEQIQLKGTVYNYRTSGMQFCVKM |
| | | | | SAVEGICTSESPVIDHQGTKSSKCVRQKVEGSSSHLVT |
| | | | | FTVLPLEIGLHNINFSLETWFGKEILVKTLRVVPEGVK |
| | | | | RESYSGVTLDPRGIYGTISRRKEFPYRIPLDLVPKTEIK |
| | | | | RILSVKGLLVGEILSAVLSQEGINILTHLPKGSAEAELM |
| | | | | SVVPVFYVFHYLETGNHWNIFHSDPLIEKQKLKKKLK |
| | | | | EGMLSIMSYRNADYSYSVWKGGSASTWLTAFALRVL |
| | | | | GQVNKYVEQNQNSICNSLLWLVENYQLDNGSFKENS |
| | | | | QYQPIKLQGTLPVEARENSLYLTAFTVIGIRKAFDICPL |
| | | | | VKIDTALIKADNFLLENTLPAQSTFTLAISAYALSLGD |
| | | | | KTHPQFRSIVSALKREALVKGNPPIYRFWKDNLQHKD |
| | | | | SSVPNTGTARMVETTAYALLTSLNLKDINYVNPVIKW |
| | | | | LSEEQRYGGGFYSTQDTINAIEGLTEYSLLVKQLRLSM |
| | | | | DIDVSYKHKGALHNYKMTDKNFLGRPVEVLLNDDLI |
| | | | | VSTGFGSGLATVHVTTVVHKTSTSEEVCSFYLKIDTQ |
| | | | | DIEASHYRGYGNSDYKRIVACASYKPSREESSSGSSHA |
| | | | | VMDISLPTGISANEEDLKALVEGVDQLFTDYQIKDGH |
| | | | | VILQLNSIPSSDFLCVRFRIFELFEVGFLSPATFTVYEYH |
| | | | | RPDKQCTMFYSTSNIKIQKVCEGAACKCVEADCGQM |
| | | | | QEELDLTISAETRKQTACKPEIAYAYKVSITSITVENVF |
| | | | | VKYKATLLDIYKTGEAVAEKDSEITFIKKVTCTNAEL |
| | | | | VKGRQYLIMGKEALQIKYNFSFRYIYPLDSLTWIEYW |
| | | | | PRDTTCSSCQAFLANLDEFAEDIFLNGC |
| SEQ | FETUA | Alpha-2-HS- | P02765 | MKSLVLLLCLAQLWGCHSAPHGPGLIYRQPNCDDPET |
| ID | | glycoprotein | | EEAALVAIDYINQNLPWGYKHTLNQIDEVKVWPQQP |
| NO: | | | | SGELFEIEIDTLETTCHVLDPTPVARCSVRQLKEHAVE |
| 209 | | | | GDCDFQLLKLDGKFSVVYAKCDSSPDSAEDVRKVCQ |
| | | | | DCPLLAPLNDTRVVHAAKAALAAFNAQNNGSNFQLE |
| | | | | EISRAQLVPLPPSTYVEFTVSGTDCVAKEATEAAKCNL |
| | | | | LAEKQYGFCKATLSEKLGGAEVAVTCMVFQTQPVSS |
| | | | | QPQPEGANEAVPTPVVDPDAPPSPPLGAPGLPPAGSPP |
| | | | | DSHVLLAAPPGHQLHRAHYDLRHTFMGVVSLGSPSG |
| | | | | EVSHPRKTRTVVQPSVGAAAGPVVPPCPGRIRHFKV |
| SEQ | HEMO | Hemopexin | P02790 | MARVLGAPVALGLWSLCWSLAIATPLPPTSAHGNVA |
| ID | | | | EGETKPDPDVTERCSDGWSFDATTLDDNGTMLFFKG |
| NO: | | | | EFVWKSHKWDRELISERWKNFPSPVDAAFRQGHNSV |
| 210 | | | | FLIKGDKVWVYPPEKKEKGYPKLLQDEFPGIPSPLDA |
| | | | | AVECHRGECQAEGVLFFQGDREWFWDLATGTMKER |
| | | | | SWPAVGNCSSALRWLGRYYCFQGNQFLRFDPVRGEV |
| | | | | PPRYPRDVRDYFMPCPGRGHGHRNGTGHGNSTHHGP |
| | | | | EYMRCSPHLVLSALTSDNHGATYAFSGTHYWRLDTS |
| | | | | RDGWHSWPIAHQWPQGPSAVDAAFSWEEKLYLVQG |
| | | | | TQVYVFLTKGGYTLVSGYPKRLEKEVGTPHGIILDSV |
| | | | | DAAFICPGSSRLHIMAGRRLWWLDLKSGAQATWTEL |
| | | | | PWPHEKVDGALCMEKSLGPNSCSANGPGLYLIHGPNL |
| | | | | YCYSDVEKLNAAKALPQPQNVTSLLGCTH |
| SEQ | HPT | Haptoglobin | P00738 | MSALGAVIALLLWGQLFAVDSGNDVTDIADDGCPKP |
| ID | | | | PEIAHGYVEHSVRYQCKNYYKLRTEGDGVYTLNDKK |
| NO: | | | | QWINKAVGDKLPECEADDGCPKPPEIAHGYVEHSVR |
| 211 | | | | YQCKNYYKLRTEGDGVYTLNNEKQWINKAVGDKLP |
| | | | | ECEAVCGKPKNPANPVQRILGGHLDAKGSFPWQAKM |
| | | | | VSHHNLTTGATLINEQWLLTTAKNLFLNHSENATAKD |
| | | | | IAPTLTLYVGKKQLVEIEKVVLHPNYSQVDIGLIKLKQ |
| | | | | KVSVNERVMPICLPSKDYAEVGRVGYVSGWGRNANF |
| | | | | KFTDHLKYVMLPVADQDQCIRHYEGSTVPEKKTPKSP |
| | | | | VGVQPILNEHTFCAGMSKYQEDTCYGDAGSAFAVHD |
| | | | | LEEDTWYATGILSFDKSCAVAEYGVYVKVTSIQDWV |
| | | | | QKTIAEN |
| SEQ | HRG | Histidine-rich | P04196 | MKALIAALLLITLQYSCAVSPTDCSAVEPEAEKALDLI |
| ID | | Glycoprotein | | NKRRRDGYLFQLLRIADAHLDRVENTTVYYLVLDVQ |
| NO: | | | | ESDCSVLSRKYWNDCEPPDSRRPSEIVIGQCKVIATRH |
| 212 | | | | SHESQDLRVIDFNCTTSSVSSALANTKDSPVLIDFFEDT |
| | | | | ERYRKQANKALEKYKEENDDFASFRVDRIERVARVR |
| | | | | GGEGTGYFVDFSVRNCPRHHFPRHPNVFGFCRADLFY |
| | | | | DVEALDLESPKNLVINCEVFDPQEHENINGVPPHLGHP |
| | | | | FHWGGHERSSTTKPPFKPHGSRDHHHPHKPHEHGPPP |
| | | | | PPDERDHSHGPPLPQGPPPLLPMSCSSCQHATFGTNGA |
| | | | | QRHSHNNNSSDLHPHKHHSHEQHPHGHHPHAHHPHE |
| | | | | HDTHRQHPHGHHPHGHHPHGHHPHGHHPHGHHPHC |
| | | | | HDFQDYGPCDPPPHNQGHCCHGHGPPPGHLRRRGPG |
| | | | | KGPRPFHCRQIGSVYRLPPLRKGEVLPLPEANFPSFPLP |
| | | | | HHKHPLKPDNQPFPQSVSESCPGKFKSGFPQVSMFFT |
| | | | | HTFPK |
| SEQ | IC1 | Plasma | P05155 | MASRLTLLTLLLLLLAGDRASSNPNATSSSSQDPESLQ |
| ID | | protease C1 | | DRGEGKVATTVISKMLFVEPILEVSSLPTTNSTTNSAT |
| NO: | | inhibitor | | KITANTTDEPTTQPTTEPTTQPTIQPTQPTTQLPTDSPT |
| 213 | | | | QPTTGSFCPGPVTLCSDLESHSTEAVLGDALVDFSLKL |
| | | | | YHAFSAMKKVETNMAFSPFSIASLLTQVLLGAGENTK |
| | | | | TNLESILSYPKDFTCVHQALKGFTTKGVTSVSQIFHSP |
| | | | | DLAIRDTFVNASRTLYSSSPRVLSNNSDANLELINTWV |
| | | | | AKNTNNKISRLLDSLPSDTRLVLLNAIYLSAKWKTTF |
| | | | | DPKKTRMEPFHFKNSVIKVPMMNSKKYPVAHFIDQTL |
| | | | | KAKVGQLQLSHNLSLVILVPQNLKHRLEDMEQALSPS |
| | | | | VFKAIMEKLEMSKFQPTLLTLPRIKVTTSQDMLSIMEK |
| | | | | LEFFDFSYDLNLCGLTEDPDLQVSAMQHQTVLELTET |
| | | | | GVEAAAASAISVARTLLVFEVQQPFLFVLWDQQHKFP |
| | | | | VFMGRVYDPRA |
| SEQ | IGA1 | Immunoglobulin | P01876 | ASPTSPKVFPLSLCSTQPDGNVVIACLVQGFFPQEPLS |
| ID | | heavy constant | | VTWSESGQGVTARNFPPSQDASGDLYTTSSQLTLPAT |
| NO: | | alpha 1 | | QCLAGKSVTCHVKHYTNPSQDVTVPCPVPSTPPTPSPS |
| 214 | | | | TPPTPSPSCCHPRLSLHRPALEDLLLGSEANLTCTLTGL |
| | | | | RDASGVTFTWTPSSGKSAVQGPPERDLCGCYSVSSVL |
| | | | | PGCAEPWNHGKTFTCTAAYPESKTPLTATLSKSGNTF |
| | | | | RPEVHLLPPPSEELALNELVTLTCLARGFSPKDVLVR |
| | | | | WLQGSQELPREKYLTWASRQEPSQGTTTFAVTSILRV |
| | | | | AAEDWKKGDTFSCMVGHEALPLAFTQKTIDRLAGKP |
| | | | | THVNVSVVMAEVDGTCY |
| SEQ | IGG1 | Immunoglobulin | P01857 | ASTKGPSVFPLAPSSKSTSGGTAALGCLVKDYFPEPVT |
| ID | | heavy constant | | VSWNSGALTSGVHTFPAVLQSSGLYSLSSVVTVPSSSL |
| NO: | | gamma 1 | | GTQTYICNVNHKPSNTKVDKKVEPKSCDKTHTCPPCP |
| 215 | | | | APELLGGPSVFLFPPKPKDTLMISRTPEVTCVVVDVSH |
| | | | | EDPEVKFNWYVDGVEVHNAKTKPREEQYNSTYRVVS |
| | | | | VLTVLHQDWLNGKEYKCKVSNKALPAPIEKTISKAK |
| | | | | GQPREPQVYTLPPSRDELTKNQVSLTCLVKGFYPSDIA |
| | | | | VEWESNGQPENNYKTTPPVLDSDGSFFLYSKLTVDKS |
| | | | | RWQQGNVFSCSVMHEALHNHYTQKSLSLSPGK |
| SEQ | IGG2 | Immunoglobulin | P01859 | ASTKGPSVFPLAPCSRSTSESTAALGCLVKDYFPEPVT |
| ID | | heavy constant | | VSWNSGALTSGVHTFPAVLQSSGLYSLSSVVTVPSSN |
| NO: | | gamma 2 | | FGTQTYTCNVDHKPSNTKVDKTVERKCCVECPPCPAP |
| 216 | | | | PVAGPSVFLFPPKPKDTLMISRTPEVTCVVVDVSHEDP |
| | | | | EVQFNWYVDGVEVHNAKTKPREEQFNSTFRVVSVLT |
| | | | | VVHQDWLNGKEYKCKVSNKGLPAPIEKTISKTKGQP |
| | | | | REPQVYTLPPSREEMTKNQVSLTCLVKGFYPSDISVE |
| | | | | WESNGQPENNYKTTPPMLDSDGSFFLYSKLTVDKSR |
| | | | | WQQGNVFSCSVMHEALHNHYTQKSLSLSPGK |
| SEQ | IGG3 | Immunoglobulin | P01860 | ASTKGPSVFPLAPCSRSTSGGTAALGCLVKDYFPEPVT |
| ID | | heavy constant | | VSWNSGALTSGVHTFPAVLQSSGLYSLSSVVTVPSSSL |
| NO: | | gamma 3 | | GTQTYTCNVNHKPSNTKVDKRVELKTPLGDTTHTCP |
| 217 | | | | RCPEPKSCDTPPPCPRCPEPKSCDTPPPCPRCPEPKSCD |
| | | | | TPPPCPRCPAPELLGGPSVFLFPPKPKDTLMISRTPEVT |
| | | | | CVVVDVSHEDPEVQFKWYVDGVEVHNAKTKPREEQ |
| | | | | YNSTFRVVSVLTVLHQDWLNGKEYKCKVSNKALPAP |
| | | | | IEKTISKTKGQPREPQVYTLPPSREEMTKNQVSLTCLV |
| | | | | KGFYPSDIAVEWESSGQPENNYNTTPPMLDSDGSFFL |
| | | | | YSKLTVDKSRWQQGNIFSCSVMHEALHNRFTQKSLSL |
| | | | | SPGK |
| SEQ | ITIH2 | Inter-alpha- | P19823 | MKRLTCFFICFFLSEVSGFEIPINGLSEFVDYEDLVELA |
| ID | | trypsin | | PGKFQLVAENRRYQRSLPGESEEMMEEVDQVTLYSY |
| NO: | | inhibitor | | KVQSTITSRMATTMIQSKVVNNSPQPQNVVFDVQIPK |
| 218 | | heavy chain | | GAFISNFSMTVDGKTFRSSIKEKTVGRALYAQARAKG |
| | | H2 | | KTAGLVRSSALDMENFRTEVNVLPGAKVQFELHYQE |
| | | | | VKWRKLGSYEHRIYLQPGRLAKHLEVDVWVIEPQGL |
| | | | | RFLHVPDTFEGHFDGVPVISKGQQKAHVSFKPTVAQQ |
| | | | | RICPNCRETAVDGELVVLYDVKREEKAGELEVENGYF |
| | | | | VHFFAPDNLDPIPKNILFVIDVSGSMWGVKMKQTVEA |
| | | | | MKTILDDLRAEDHFSVIDFNQNIRTWRNDLISATKTQ |
| | | | | VADAKRYIEKIQPSGGTNINEALLRAIFILNEANNLGLL |
| | | | | DPNSVSLIILVSDGDPTVGELKLSKIQKNVKENIQDNIS |
| | | | | LFSLGMGFDVDYDFLKRLSNENHGIAQRIYGNQDTSS |
| | | | | QLKKFYNQVSTPLLRNVQFNYPHTSVTDVTQNNFHN |
| | | | | YFGGSEIVVAGKFDPAKLDQIESVITATSANTQLVLET |
| | | | | LAQMDDLQDFLSKDKHADPDFTRKLWAYLTINQLLA |
| | | | | ERSLAPTAAAKRRITRSILQMSLDHHIVTPLTSLVIENE |
| | | | | AGDERMLADAPPQDPSCCSGALYYGSKVVPDSTPSW |
| | | | | ANPSPTPVISMLAQGSQVLESTPPPHVMRVENDPHFII |
| | | | | YLPKSQKNICFNIDSEPGKILNLVSDPESGIVVNGQLV |
| | | | | GAKKPNNGKLSTYFGKLGFYFQSEDIKIEISTETITLSH |
| | | | | GSSTFSLSWSDTAQVTNQRVQISVKKEKVVTITLDKE |
| | | | | MSFSVLLHRVWKKHPVNVDFLGIYIPPTNKFSPKAHG |
| | | | | LIGQFMQEPKIHIFNERPGKDPEKPEASMEVKGQKLIIT |
| | | | | RGLQKDYRTDLVFGTDVTCWFVHNSGKGFIDGHYKD |
| | | | | YFVPQLYSFLKRP |
| SEQ | KLKB1 | Plasma | P03952 | MILFKQATYFISLFATVSCGCLTQLYENAFFRGGDVAS |
| ID | | Kallikrein | | MYTPNAQYCQMRCTFHPRCLLFSFLPASSINDMEKRF |
| NO: | | | | GCFLKDSVTGTLPKVHRTGAVSGHSLKQCGHQISACH |
| 219 | | | | RDIYKGVDMRGVNFNVSKVSSVEECQKRCTNNIRCQ |
| | | | | FFSYATQTFHKAEYRNNCLLKYSPGGTPTAIKVLSNV |
| | | | | ESGFSLKPCALSEIGCHMNIFQHLAFSDVDVARVLTPD |
| | | | | AFVCRTICTYHPNCLFFTFYTNVWKIESQRNVCLLKTS |
| | | | | ESGTPSSSTPQENTISGYSLLTCKRTLPEPCHSKIYPGV |
| | | | | DFGGEELNVTFVKGVNVCQETCTKMIRCQFFTYSLLP |
| | | | | EDCKEEKCKCFLRLSMDGSPTRIAYGTQGSSGYSLRL |
| | | | | CNTGDNSVCTTKTSTRIVGGTNSSWGEWPWQVSLQV |
| | | | | KLTAQRHLCGGSLIGHQWVLTAAHCFDGLPLQDVWR |
| | | | | IYSGILNLSDITKDTPFSQIKEIIIHQNYKVSEGNHDIALI |
| | | | | KLQAPLNYTEFQKPICLPSKGDTSTIYTNCWVTGWGF |
| | | | | SKEKGEIQNILQKVNIPLVTNEECQKRYQDYKITQRM |
| | | | | VCAGYKEGGKDACKGDSGGPLVCKHNGMWRLVGIT |
| | | | | SWGEGCARREQPGVYTKVAEYMDWILEKTQSSDGK |
| | | | | AQMQSPA |
| SEQ | SEPP1 | Selenoprotein | P49908 | MWRSLGLALALCLLPSGGTESQDQSSLCKQPPAWSIR |
| ID | | P | | DQDPMLNSNGSVTVVALLQASUYLCILQASKLEDLR |
| NO: | | | | VKLKKEGYSNISYIVVNHQGISSRLKYTHLKNKVSEHI |
| 220 | | | | PVYQQEENQTDVWTLLNGSKDDFLIYDRCGRLVYHL |
| | | | | GLPFSFLTFPYVEEAIKIAYCEKKCGNCSLTTLKDEDF |
| | | | | CKRVSLATVDKTVETPSPHYHHEHHHNHGHQHLGSS |
| | | | | ELSENQQPGAPNAPTHPAPPGLHHHHKHKGQHRQGH |
| | | | | PENRDMPASEDLQDLQKKLCRKRCINQLLCKLPTDSE |
| | | | | LAPRSUCCHCRHLIFEKTGSAITUQCKENLPSLCSUQG |
| | | | | LRAEENITESCQURLPPAAUQISQQLIPTEASASURUK |
| | | | | NQAKKUEUPSN |
| SEQ | TRFE | Serotransferrin | P02787 | MRLAVGALLVCAVLGLCLAVPDKTVRWCAVSEHEA |
| ID | | | | TKCQSFRDHMKSVIPSDGPSVACVKKASYLDCIRAIA |
| NO: | | | | ANEADAVTLDAGLVYDAYLAPNNLKPVVAEFYGSKE |
| 221 | | | | DPQTFYYAVAVVKKDSGFQMNQLRGKKSCHTGLGR |
| | | | | SAGWNIPIGLLYCDLPEPRKPLEKAVANFFSGSCAPCA |
| | | | | DGTDFPQLCQLCPGCGCSTLNQYFGYSGAFKCLKDG |
| | | | | AGDVAFVKHSTIFENLANKADRDQYELLCLDNTRKP |
| | | | | VDEYKDCHLAQVPSHTVVARSMGGKEDLIWELLNQA |
| | | | | QEHFGKDKSKEFQLFSSPHGKDLLFKDSAHGFLKVPP |
| | | | | RMDAKMYLGYEYVTAIRNLREGTCPEAPTDECKPVK |
| | | | | WCALSHHERLKCDEWSVNSVGKIECVSAETTEDCIAK |
| | | | | IMNGEADAMSLDGGFVYIAGKCGLVPVLAENYNKSD |
| | | | | NCEDTPEAGYFAIAVVKKSASDLTWDNLKGKKSCHT |
| | | | | AVGRTAGWNIPMGLLYNKINHCRFDEFFSEGCAPGSK |
| | | | | KDSSLCKLCMGSGLNLCEPNNKEGYYGYTGAFRCLV |
| | | | | EKGDVAFVKHQTVPQNTGGKNPDPWAKNLNEKDYE |
| | | | | LLCLDGTRKPVEEYANCHLARAPNHAVVTRKDKEAC |
| | | | | VHKILRQQQHLFGSNVTDCSGNFCLFRSETKDLLFRD |
| | | | | DTVCLAKLHDRNTYEKYLGEEYVKAVGNLRKCSTSS |
| | | | | LLEACTFRRP |
| SEQ | ZA2G | Zinc-alpha-2- | P25311 | MVRMVPVLLSLLLLLGPAVPQENQDGRYSLTYIYTGL |
| ID | | glycoprotein | | SKHVEDVPAFQALGSLNDLQFFRYNSKDRKSQPMGL |
| NO: | | | | WRQVEGMEDWKQDSQLQKAREDIFMETLKDIVEYY |
| 222 | | | | NDSNGSHVLQGRFGCEIENNRSSGAFWKYYYDGKDY |
| | | | | IEFNKEIPAWVPFDPAAQITKQKWEAEPVYVQRAKAY |
| | | | | LEEECPATLRKYLKYSKNILDRQDPPSVVVTSHQAPG |
| | | | | EKKKLKCLAYDFYPGKIDVHWTRAGEVQEPELRGDV |
| | | | | LHNGNGTYQSWVVVAVPPQDTAPYSCHVQHSSLAQP |
| | | | | LVVPWEAS |
| SEQ | IGA2 | Immunoglobulin | P01877 | ASPTSPKVFPLSLDSTPQDGNVVVACLVQGFFPQEPLS |
| ID | | heavy constant | | VTWSESGQNVTARNFPPSQDASGDLYTTSSQLTLPAT |
| NO: | | alpha 2 | | QCPDGKSVTCHVKHYTNSSQDVTVPCRVPPPPPCCHP |
| 223 | | | | RLSLHRPALEDLLLGSEANLTCTLTGLRDASGATFTW |
| | | | | TPSSGKSAVQGPPERDLCGCYSVSSVLPGCAQPWNHG |
| | | | | ETFTCTAAHPELKTPLTANITKSGNTFRPEVHLLPPPSE |
| | | | | ELALNELVTLTCLARGFSPKDVLVRWLQGSQELPREK |
| | | | | YLTWASRQEPSQGTTTYAVTSILRVAAEDWKKGETFS |
| | | | | CMVGHEALPLAFTQKTIDRMAGKPTHINVSVVMAEA |
| | | | | DGTCY |

Table 35 includes the Peptide Structure (PS) Name (e.g., A2GL_186_5402), which is a reference code for the protein name (e.g., A2GL), followed by the glycan linking site position in the protein (e.g., the number 186 that is preceded by an underscore and represents a sequential amino acid position in protein A2GL), and followed by the glycan structure GL number (e.g., the number 5402 that is preceded by an underscore and represents a glycan composition Hex(5)HexNAc(4)Fuc(0)NeuAc(2)). The Peptide Structure (PS) Name of Table 35 contains a prefix that represents an abbreviation (that may include a combination of letters and numbers) for a protein abbreviation that corresponds to the Protein Abbreviation of Table 34. The term Glycan Linking Site Pos. in Protein Sequence is a number that refers to the sequential position of an amino acid of the corresponding protein in which a glycan is attached. For the Glycan Linking Site Pos. in Protein Sequence, the amino acid position of the peptide sequence is defined by the sequentially numbered order of amino acids based on the Uniprot ID of the corresponding protein for the peptide sequence. The term Glycan Linking Site Pos. in Peptide Sequence is a number that refers to the sequential position of an amino acid of the corresponding peptide in which a glycan is attached. For the Glycan Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Tables 36A and 36B.

TABLE 35
Details of peptides and glycopeptides
with statistically significant changing
abundances in healthy and NSCLC samples

| | | | Glycan | Glycan | |
|---|---|---|---|---|---|
| | | | Linking | Linking | |
| | | | Site | Site | |
| | | | Pos. in | Pos. in | Glycan |
| SEQ ID | Peptide Structure | Peptide | Peptide | Protein | Structure |
| NO | (PS) NAME | Sequence | Sequence | Sequence | GL NO |
| SEQ ID | A2GL_186_5402 | LPPGLLANFTLL | 8 | 186 | 5402 |
| NO: 224 | | R | | | |
| SEQ ID | A2MG_1424_6511 | VSNQTLSLFFTV | 3 | 1424 | 6511 |
| NO: 225 | | LQDVPVR | | | |
| SEQ ID | A2MG | VSNQTLSLFFTV | N/A | N/A | Non- |
| NO: 226 | | LQDVPVR | | | Glycosylated |
| SEQ ID | A2MG_869_5402 | SLGNVNFTVSAE | 6 | 869 | 5402 |
| NO: 227 | | ALESQELCGTEV | | | |
| | | PSVPEHGR | | | |
| SEQ ID | AACT_127_6503 | TLNQSSDELQLS | 3 | 127 | 6503 |
| NO: 228 | | MGNAMFVK | | | |
| SEQ ID | AACT_271_6513 | YTGNASALFILP | 4 | 271 | 6513 |
| NO: 229 | | DQDK | | | |
| SEQ ID | APOB_3411_5301 | FVEGSHNSTVSL | 7 | 3411 | 5301 |
| NO: 230 | | TTK | | | |
| SEQ ID | C1S_174_5402 | NCGVNCSGDVF | 5 | 174 | 5402 |
| NO: 231 | | TALIGEIASPNYP | | | |
| | | KPYPENSR | | | |
| SEQ ID | CFAI_70_5402 | NGTAVCATNR | 1 | 70 | 5402 |
| NO: 232 | | | | | |
| SEQ ID | CO5_741_5401 | ANISHK | 2 | 741 | 5401 |
| NO: 233 | | | | | |
| SEQ ID | FETUA_176_5402 | AALAAFNAQNN | 11 | 176 | 5402 |
| NO: 234 | | GSNFQLEEISR | | | |
| SEQ ID | FETUA | TVVQPSVGAAA | N/A | N/A | Non- |
| NO: 235 | | GPVVPPCPGR | | | Glycosylated |
| SEQ ID | HEMO_187_6513 | SWPAVGNCSSA | 7 | 187 | 6513 |
| NO: 236 | | LR | | | |
| SEQ ID | HEMO_453_5402 | ALPQPQNVTSLL | 7 | 453 | 5402 |
| NO: 237 | | GCTH | | | |
| SEQ ID | HPT_184_6501 | MVSHHNLTTGA | 6 | 184 | 6501 |
| NO: 238 | | TLINEQWLLTTA | | | |
| | | K | | | |
| SEQ ID | HPT_184_6502 | MVSHHNLTTGA | 6 | 184 | 6502 |
| NO: 239 | | TLINEQWLLTTA | | | |
| | | K | | | |
| SEQ ID | HPT_241_6512 | VVLHPNYSQVDI | 6 | 241 | 6512 |
| NO: 240 | | GLIK | | | |
| SEQ ID | IC1_238_5402 | DTFVNASR | 5 | 238 | 5402 |
| NO: 241 | | | | | |
| SEQ ID | IC1_253_6513 | VLSNNSDANLEL | 4 | 253 | 6513 |
| NO: 242 | | INTWVAK | | | |
| SEQ ID | IGA12_144MC_3500 | PALEDLLLGSEA | 13 | 144 | 3500 |
| NO: 243 | | NLTCTLTGLR | | | |
| SEQ ID | IGG1_297_3510 | EEQYNSTYR | 5 | 180 | 3510 |
| NO: 244 | | | | | |
| SEQ ID | IGG1_297_4500 | EEQYNSTYR | 5 | 180 | 4500 |
| NO: 245 | | | | | |
| SEQ ID | IGG1_297_5410 | EEQYNSTYR | 5 | 180 | 5410 |
| NO: 246 | | | | | |
| SEQ ID | IGG2_297_3500 | EEQFNSTFR | 5 | 175 | 3500 |
| NO: 247 | | | | | |
| SEQ ID | IGG2_297_4410 | EEQFNSTFR | 5 | 175 | 4410 |
| NO: 248 | | | | | |
| SEQ ID | IGG2_297_5410 | EEQFNSTFR | 5 | 175 | 5410 |
| NO: 249 | | | | | |
| SEQ ID | KLKB1 | IYSGILNLSDITK | N/A | N/A | Non- |
| NO: 250 | | | | | Glycosylated |
| SEQ ID | KLKB1_494_5410 | LQAPLNYTEFQK | 6 | 494 | 5410 |
| NO: 251 | | PICLPSK | | | |
| SEQ ID | IGG3 | TPEVTCVVVDVS | N/A | N/A | Non- |
| NO: 252 | | HEDPEVQFK | | | Glycosylated |
| SEQ ID | APOM | AFLLTPR | N/A | N/A | Non- |
| NO: 253 | | | | | Glycosylated |
| SEQ ID | B2M | VNHVTLSQPK | N/A | N/A | Non- |
| NO: 254 | | | | | Glycosylated |
| SEQ ID | SEPP1 | VSLATVDK | N/A | N/A | Non- |
| NO: 255 | | | | | Glycosylated |
| SEQ ID | TRFE_630_5402_ | QQQHLFGSNVT | 9 | 630 | 5402 |
| NO: 256 | NH3LOSS | DCSGNFCLFR | | | |
| SEQ ID | ZA2G_128_5402 | FGCEIENNR | 8 | 128 | 5402 |
| NO: 257 | | | | | |
| SEQ ID | A2MG_1424_5411 | VSNQTLSLFFTV | 3 | 1424 | 5411 |
| NO: 258 | | LQDVPVR | | | |
| SEQ ID | A2MG_1424_5412 | VSNQTLSLFFTV | 3 | 1424 | 5412 |
| NO: 259 | | LQDVPVR | | | |
| SEQ ID | A2MG_247_5200 | IITILEEEMNVSV | 10 | 247 | 5200 |
| NO: 260 | | CGLYTYGKPVP | | | |
| | | GHVTVSICR | | | |
| SEQ ID | A2MG_247_5401 | IITILEEEMNVSV | 10 | 247 | 5401 |
| NO: 261 | | CGLYTYGKPVP | | | |
| | | GHVTVSICR | | | |
| SEQ ID | A2MG_247MC_5401 | IITILEEEMNVSV | 10 | 247 | 5401 |
| NO: 262 | | CGLYTYGK | | | |
| SEQ ID | A2MG_869_5401 | SLGNVNFTVSAE | 6 | 869 | 5401 |
| NO: 263 | | ALESQELCGTEV | | | |
| | | PSVPEHGR | | | |
| SEQ ID | AGP12_72_6503 | SVQEIQATFFYFT | 15 | 72 | 6503 |
| NO: 264 | | PNK | | | |
| SEQ ID | AGP12_72_7603 | SVQEIQATFFYFT | 15 | 72 | 7603 |
| NO: 265 | | PNK | | | |
| SEQ ID | AGP12_72_7604 | SVQEIQATFFYFT | 15 | 72 | 7604 |
| NO: 266 | | PNK | | | |
| SEQ ID | AGP12_72MC_6503 | SVQEIQATFFYFT | 15 | 72 | 6503 |
| NO: 267 | | PNKTEDTIFLR | | | |
| SEQ ID | AGP12_72MC_7603 | SVQEIQATFFYFT | 15 | 72 | 7603 |
| NO: 268 | | PNKTEDTIFLR | | | |
| SEQ ID | AGP2_103_6513 | ENGTVSR | 2 | 103 | 6513 |
| NO: 269 | | | | | |
| SEQ ID | AGP2_103_7614 | ENGTVSR | 2 | 103 | 7614 |
| NO: 270 | | | | | |
| SEQ ID | APOB_983_5402 | QVFPGLNYCTSG | 16 | 983 | 5402 |
| NO: 271 | | AYSNASSTDSAS | | | |
| | | YYPLTGDTR | | | |
| SEQ ID | CERU_138_6513 | EHEGAIYPDNTT | 10 | 138 | 6513 |
| NO: 272 | | DFQR | | | |
| SEQ ID | CERU_762_6513 | ELHHLQEQNVS | 9 | 762 | 6513 |
| NO: 273 | | NAFLDK | | | |
| SEQ ID | FETUA_176_6501 | AALAAFNAQNN | 11 | 176 | 6501 |
| NO: 274 | | GSNFQLEEISR | | | |
| SEQ ID | FETUA_176_6513 | AALAAFNAQNN | 11 | 176 | 6513 |
| NO: 275 | | GSNFQLEEISR | | | |
| SEQ ID | HPT_207_121015 | NLFLNHSENATA | 5, | 207, | 6513, |
| NO: 276 | | K | 9 | 211 | 6502 |
| SEQ ID | HPT_207_5401 | NLFLNHSENATA | 5 | 207 | 5401 |
| NO: 277 | | K | | | |
| SEQ ID | HPT_241_6500 | VVLHPNYSQVDI | 6 | 241 | 6500 |
| NO: 278 | | GLIK | | | |
| SEQ ID | HPT_241_6511 | VVLHPNYSQVDI | 6 | 241 | 6511 |
| NO: 279 | | GLIK | | | |
| SEQ ID | HRG_125_5402 | VIDFNCTTSSVSS | 5 | 125 | 5402 |
| NO: 280 | | ALANTK | | | |
| SEQ ID | HRG_271_1102 | SSTTKPPFKPHGS | 1 | 271 | 1102 |
| NO: 281 | | R | | | |
| SEQ ID | HRG_271_2201 | SSTTKPPFKPHGS | 1 | 271 | 2201 |
| NO: 282 | | R | | | |
| SEQ ID | IGA12_144_5400 | LSLHRPALEDLL | 18 | 144 | 5400 |
| NO: 283 | | LGSEANLTCTLT | | | |
| | | GLR | | | |
| SEQ ID | IGA12_144_5401 | LSLHRPALEDLL | 18 | 144 | 5401 |
| NO: 284 | | LGSEANLTCTLT | | | |
| | | GLR | | | |
| SEQ ID | IGA12_144MC_4501 | PALEDLLLGSEA | 13 | 144 | 4501 |
| NO: 285 | | NLTCTLTGLR | | | |
| SEQ ID | IGA12_144MC_5401 | PALEDLLLGSEA | 13 | 144 | 5401 |
| NO: 286 | | NLTCTLTGLR | | | |
| SEQ ID | IGG1_297_3410 | EEQYNSTYR | 5 | 180 | 3410 |
| NO: 287 | | | | | |
| SEQ ID | IGG1_297_4310 | EEQYNSTYR | 5 | 180 | 4310 |
| NO: 288 | | | | | |
| SEQ ID | IGG1_297_4410_Z4 | EEQYNSTYR | 5 | 180 | 4410 |
| NO: 289 | | | | | |
| SEQ ID | IGG2 | EEQFNSTFR | N/A | N/A | Non- |
| NO: 290 | | | | | Glycosylated |
| SEQ ID | ITIH2_118_5402 | GAFISNFSMTVD | 6 | 118 | 5402 |
| NO: 291 | | GK | | | |
| SEQ ID | KLKB1_127_5402 | GVNFNVSK | 5 | 127 | 5402 |
| NO: 292 | | | | | |
| SEQ ID | KLKB1_494_6503 | LQAPLNYTEFQK | 6 | 494 | 6503 |
| NO: 293 | | PICLPSK | | | |
| SEQ ID | TRFE_630_5400 | QQQHLFGSNVT | 9 | 630 | 5400 |
| NO: 294 | | DCSGNFCLFR | | | |
| SEQ ID | TRFE_630_5402 | QQQHLFGSNVT | 9 | 630 | 5402 |
| NO: 295 | | DCSGNFCLFR | | | |
| SEQ ID | TRFE_630_6513 | QQQHLFGSNVT | 9 | 630 | 6513 |
| NO: 296 | | DCSGNFCLFR | | | |

In some embodiments, the term AGP 12 for SEQ ID NOs: 67-71 represent that the glycopeptide is a fragment of either AGP1 or AGP2. In some embodiments, the term IGA12 for SEQ ID NOs: 243, 283-286 represent that the glycopeptide is a fragment of either IGA1 or IGA2. For the SEQ ID NO:276 in Table 35, the identity of the glycopeptide is one of two possibilities that have the same monoisotopic mass. In the first possibility, the glycan having the Glycan GL NO 6513 is attached to the peptide with a Glycan linking site position of 5 in the peptide sequence. In the second possibility, the glycan having the Glycan GL NO 6502 is attached to the peptide with a Glycan linking site position of 9 in the peptide sequence.

In Table 35, if the first number subsequent to the first underscore in the Peptide Structure (PS) NAME is inconsistent with the Glycan Linking Site Pos. in Protein Sequence column, then the Glycan Linking Site Pos. in Protein Sequence column should be used for identification of the peptide. In Table 35, if the second number subsequent to the second underscore in the Peptide Structure (PS) NAME is inconsistent with the Glycan Structure GL NO column, then the Glycan Structure GL NO column should be used for identification of the glycan portion of the glycopeptide. If the Peptide Structure (PS) NAME does not contain any numbers, then the peptide is non-glycosylated. In some instances of the Peptide Structure (PS) NAME, subsequent to the prefix, there is a number noted with the notation MC that indicates that there was a missed cleavage at position in the peptide sequence as noted by the number. In some instances of the Peptide Structure (PS) NAME, there is a suffix NH3LOSS to indicate a loss of a NH₃ group.

Tables 36A and 36B illustrate the symbol structure and composition of detected glycan moieties that correspond to glycopeptides of Table 35 based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. It should be noted that the Glycan Structure GL NOs. 1102 and 2201 are O-linked glycans that are in Table 36A and that N-linked glycans are in Table 36B. For reference, N-linked glycans have a glycan attached to the amino acid asparagine and O-linked glycans have a glycan attached to either a serine or a threonine.

The identity of the various monosaccharides is illustrated by the Legend section located at the end of Table 36B. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

Referring back to Tables 36A and 36B, for some entries, there are two symbol structures provided for one Glycan Structure GL NO such as, for example, Glycan Structure GL NO 4500. Thus, the identify of a peptide that references a Glycan Structure GL NO that has two symbol structures could one of two possibilities based on the MRM of the LC-MS analysis. In some instances, a bracket symbol is used as part of the Symbol Structure to indicate that the precise bonding linkage is not exactly known, but that the linking line segment is attached to one of the plurality of adjacent carbohydrates immediately adjacent to the bracket

TABLE 36A
Glycan structure GL NO, symbol structure, and composition
of detected glycan moieties for O-linked glycans

| Glycan Structure | Symbol | |
|---|---|---|
| GL NO. | Structure | Composition |
| 1102 | [symbol] | Hex(1)HexNAc(1)Fuc(0)NeuAc(2) |
| 2201 | [symbol] | Hex(2)HexNAc(2)Fuc(0)NeuAc(1) |

TABLE 36B
Glycan structure GL NO, symbol structure, and composition
of detected glycan moieties for N-linked glycans

| Glycan Structure | Symbol | |
|---|---|---|
| GL NO. | Structure | Composition |
| 3410 | [symbol] | Hex(3)HexNAc(4)Fuc(1)NeuAc(0) |
| 3500 | [symbol] | Hex(3)HexNAc(5)Fuc(0)NeuAc(0) |
| 3510 | [symbol] | Hex(3)HexNAc(5)Fuc(1)NeuAc(0) |
| 4310 | [symbol] | Hex(4)HexNAc(3)Fuc(1)NeuAc(0) |
| 4410 | [symbol] | Hex(4)HexNAc(4)Fuc(1)NeuAc(0) |
| 4500 | [symbol] | Hex(4)HexNAc(5)Fuc(0)NeuAc(0) |
| 4501 | [symbol] | Hex(4)HexNAc(5)Fuc(0)NeuAc(1) |
| 5200 | [symbol] | Hex(5)HexNAc(2)Fuc(0)NeuAc(0) |
| 5301 | [symbol] | Hex(5)HexNAc(3)Fuc(0)NeuAc(1) |
| 5400 | [symbol] | Hex(5)HexNAc(4)Fuc(0)NeuAc(0) |
| 5401 | [symbol] | Hex(5)HexNAc(4)Fuc(0)NeuAc(1) |
| 5402 | [symbol] | Hex(5)HexNAc(4)Fuc(0)NeuAc(2) |
| 5410 | [symbol] | Hex(5)HexNAc(4)Fuc(1)NeuAc(0) |
| 5411 | [symbol] | Hex(5)HexNAc(4)Fuc(1)NeuAc(1) |
| 5412 | [symbol] | Hex(5)HexNAc(4)Fuc(1)NeuAc(2) |
| 6500 | [symbol] | Hex(6)HexNAc(5)Fuc(0)NeuAc(0) |
| 6501 | [symbol] | Hex(6)HexNAc(5)Fuc(0)NeuAc(1) |
| 6502 | [symbol] | Hex(6)HexNAc(5)Fuc(0)NeuAc(2) |
| 6503 | [symbol] | Hex(6)HexNAc(5)Fuc(0)NeuAc(3) |
| 6511 | [symbol] | Hex(6)HexNAc(5)Fuc(1)NeuAc(1) |
| 6512 | [symbol] | Hex(6)HexNAc(5)Fuc(1)NeuAc(2) |
| 6513 | [symbol] | Hex(6)HexNAc(5)Fuc(1)NeuAc(3) |
| 7603 | [symbol] | Hex(7)HexNAc(6)Fuc(0)NeuAc(3) |
| 7604 | [symbol] | Hex(7)HexNAc(6)Fuc(0)NeuAc(4) |
| 7614 | [symbol] | Hex(7)HexNAc(6)Fuc(1)NeuAc(4) |

- Legend for Tables 36A and 36B
● Glc [symbol]
Gal [symbol]
Man [symbol]
Fuc [symbol]
Neu5Ac
▪ GlcNAc [symbol]
GalNAc [symbol]
ManNAc

TABLE 37
MRM-MS and liquid chromatography (LC) parameters for peptide structures
associated with healthy control and NSCLC populations

| SEQ | | | Collision | 1st | 1st | 2nd | 2nd | 1st | 2nd |
|---|---|---|---|---|---|---|---|---|---|
| ID | Monoisotopic | RT | Energy | Precursor | Precursor | Precursor | Precursor | Product | Product |
| NO | mass | (min) | (V) | m/z | Charge | m/z | Charge | m/z | m/z |
| 224 | 3628.627482 | 40.2 | 32 | 1210.9 | 3 | 908.4 | 4 | 366.1 | 366.1 |
| 258 | 4221.908394 | 43.5 | 22 | 1057 | 4 | 1057 | 4 | 366.1 | 1184.1 |
| 259 | 4513.003804 | 44 | 23 | 1129.7 | 4 | 1129.7 | 4 | 366.1 | 1183.6 |
| 225 | 4587.040582 | 43.3 | 23 | 1148.3 | 4 | 1148.3 | 4 | 366.1 | 1183.6 |
| 226 | 2162.173506 | 44.8 | 10 | 721.8 | 3 | 721.8 | 3 | 371.24 | 826.48 |
| 260 | 4950.310912 | 38.8 | 25 | 1239.1 | 4 | N/A | N/A | 1314.2 | N/A |
| 261 | 5647.565058 | 39.3 | 30 | 1413.6 | 4 | 1131.1 | 5 | 366.1 | 1313.3 |
| 262 | 4344.878414 | 40.165 | 30 | 1450 | 3 | 1450 | 3 | 366.1 | 1318.7 |
| 263 | 5326.297412 | 35.1 | 22 | 1066.7 | 5 | 1066.7 | 5 | 366.1 | 1206.6 |
| 227 | 5617.392822 | 35.9 | 25 | 1124.9 | 5 | 1124.9 | 5 | 366.1 | 1206.2 |
| 228 | 5073.050342 | 34.1 | 35 | 1269.8 | 4 | 1269.8 | 4 | 366.1 | 1208.6 |
| 229 | 4758.930872 | 31.3 | 30 | 1191.2 | 4 | 1191.2 | 4 | 366.1 | 978.5 |
| 264 | 4779.946464 | 37.7 | 30 | 1196.5 | 4 | 1196.5 | 4 | 366.1 | 1062.5 |
| 265 | 5145.078652 | 37.5 | 25 | 1287.8 | 4 | 1287.8 | 4 | 366.1 | 1062.5 |
| 266 | 5436.174062 | 37.8 | 30 | 1360.5 | 4 | 1360.5 | 4 | 366.1 | 1062.5 |
| 267 | 5755.448988 | 41 | 28 | 1152.7 | 5 | 1152.7 | 5 | 366.1 | 1550.3 |
| 268 | 6120.581176 | 40.9 | 23 | 1531.9 | 4 | 1225.7 | 5 | 366.1 | 1550.3 |
| 269 | 3768.424656 | 4.6 | 33 | 1257.5 | 3 | 1257.5 | 3 | 366.1 | 965.5 |
| 270 | 4424.652254 | 4.7 | 30 | 1107.4 | 4 | 1107.4 | 4 | 366.1 | 965.5 |
| 230 | 3316.397426 | 12.6 | 28 | 1106.8 | 3 | N/A | N/A | 366.1 | N/A |
| 271 | 5754.335366 | 33.6 | 30 | 1440.3 | 4 | N/A | N/A | 366.1 | N/A |
| 231 | 5730.401612 | 41 | 25 | 1147.8 | 5 | 1434.3 | 4 | 366.1 | 366.1 |
| 272 | 4898.89152 | 17.1 | 30 | 1226.2 | 4 | 1226.2 | 4 | 366.1 | 1048.5 |
| 273 | 5028.0545 | 20.8 | 30 | 1258.5 | 4 | 1258.5 | 4 | 274.1 | 1113 |
| 232 | 3267.260004 | 6.4 | 34 | 1090.1 | 3 | N/A | N/A | 366.1 | N/A |
| 233 | 2582.037538 | 3.9 | 20 | 862 | 3 | N/A | N/A | 366.1 | N/A |
| 234 | 4568.91817 | 30.4 | 28 | 1143.7 | 4 | N/A | N/A | 366.1 | N/A |
| 274 | 4642.954948 | 29.8 | 29 | 1161.7 | 4 | N/A | N/A | 366.1 | N/A |
| 275 | 5371.203674 | 30.4 | 34 | 1343.8 | 4 | N/A | N/A | 366.1 | N/A |
| 235 | 2015.062182 | 23.65 | 15 | 672.8 | 3 | 672.8 | 3 | 683.33 | 586.28 |
| 236 | 4410.719444 | 21.5 | 25 | 1104.2 | 4 | N/A | N/A | 274.1 | N/A |
| 237 | 3939.644654 | 30.6 | 30 | 1314.9 | 3 | N/A | N/A | 366.1 | N/A |
| 238 | 4957.194128 | 32.2 | 20 | 992.8 | 5 | N/A | N/A | 366.1 | N/A |
| 239 | 5248.289538 | 33.5 | 26 | 1051 | 5 | 1051 | 5 | 366.1 | 1441.7 |
| 276 | 7034.688732 | 13.2 | 29 | 1173.6 | 6 | N/A | N/A | 366.1 | N/A |
| 277 | 3371.403238 | 13.5 | 30 | 1124.8 | 3 | 1124.8 | 3 | 366.1 | 831.4 |
| 278 | 3781.71769 | 27.6 | 30 | 1261.6 | 3 | 1261.6 | 3 | 366.1 | 999.5 |
| 279 | 4218.871006 | 28.9 | 25 | 1055.7 | 4 | 1055.7 | 4 | 366.1 | 999.5 |
| 240 | 4509.966416 | 30 | 28 | 1128.8 | 4 | N/A | N/A | 366.1 | N/A |
| 280 | 4218.740064 | 28.4 | 25 | 1056.2 | 4 | 1407.9 | 3 | 366.1 | 366.1 |
| 281 | 2473.123096 | 7.3 | 15 | 619.5 | 4 | N/A | N/A | 274.1 | N/A |
| 282 | 2547.159874 | 5.5 | 25 | 638 | 4 | 638 | 4 | 274.1 | 865.4 |
| 241 | 3113.207564 | 10.2 | 25 | 1039.1 | 3 | 1039.1 | 3 | 366.1 | 1112.5 |
| 242 | 5107.14298 | 35.7 | 40 | 1278.3 | 4 | 1278.3 | 4 | 204.1 | 1152.6 |
| 283 | 4585.172492 | 40.2 | 28 | 1147.3 | 4 | N/A | N/A | 204.1 | N/A |
| 284 | 4876.267902 | 40.8 | 20 | 976.3 | 5 | N/A | N/A | 366.1 | N/A |
| 243 | 3857.786056 | 42 | 27 | 1287.6 | 3 | 1287.6 | 3 | 204.1 | 1281.2 |
| 285 | 4310.934286 | 42.4 | 35 | 1079.2 | 4 | 1079.2 | 4 | 204.1 | 1281.2 |
| 286 | 4269.907738 | 42.4 | 20 | 1069 | 4 | 1069 | 4 | 366.1 | 1281.2 |
| 287 | 2633.03854 | 7.9 | 21 | 879 | 3 | 879 | 3 | 204.1 | 1392.6 |
| 244 | 2836.117908 | 8.1 | 15 | 946.5 | 3 | 946.5 | 3 | 204.1 | 1392.6 |
| 288 | 2592.011992 | 7.8 | 35 | 1297.5 | 2 | N/A | N/A | 366.1 | N/A |
| 289 | 2795.09136 | 7.8 | 15 | 699.8 | 4 | 699.8 | 4 | 204.1 | 1392.6 |
| 245 | 2852.112822 | 8.1 | 23 | 951.7 | 3 | 951.7 | 3 | 204.1 | 1392.6 |
| 246 | 2957.14418 | 7.8 | 24 | 987.1 | 3 | 987.1 | 3 | 366.1 | 1392.6 |
| 247 | 2658.070174 | 13.1 | 30 | 887.4 | 3 | N/A | N/A | 1360.6 | N/A |
| 248 | 2763.101532 | 13 | 22 | 922.1 | 3 | 922.1 | 3 | 204.1 | 1360.6 |
| 249 | 2925.154352 | 12.7 | 20 | 976.1 | 3 | N/A | N/A | 366.1 | N/A |
| 290 | 1156.514874 | 14.93 | 16 | 579.6 | 2 | 579.6 | 2 | 624.3 | 510.3 |
| 291 | 3677.469324 | 29.2 | 30 | 1227.5 | 3 | N/A | N/A | 366.1 | N/A |
| 292 | 3068.222488 | 14 | 25 | 1024.1 | 3 | 1024.1 | 3 | 366.1 | 1067.5 |
| 250 | 1435.792214 | 35.295 | 20 | 719.3 | 2 | 719.3 | 2 | 1160.7 | 903.5 |
| 251 | 4014.816354 | 30.4 | 20 | 1004.7 | 4 | N/A | N/A | 366.1 | N/A |
| 293 | 5107.176866 | 33.5 | 38 | 1277.8 | 4 | N/A | N/A | 366.1 | N/A |
| 252 | 2413.147086 | 31.9 | 25 | 805.4 | 3 | N/A | N/A | 994.5 | 1044 |
| 253 | 816.485756 | 23.2 | 10 | 409.2 | 2 | N/A | N/A | 599.4 | 486.3 |
| 254 | 1121.61928 | 9.5 | 25 | 561.8 | 2 | N/A | N/A | 244.2 | 455.3 |
| 255 | 831.470164 | 12.7 | 10 | 416.7 | 2 | N/A | N/A | 646.4 | 733.4 |
| 294 | 4136.698372 | 30.4 | 25 | 1035.6 | 4 | N/A | N/A | 366.1 | N/A |
| 295 | 4718.889192 | 32.7 | 29 | 1181.1 | 4 | N/A | N/A | 366.1 | N/A |
| 256 | 4701.863 | 35.2 | 29 | 1177 | 4 | N/A | N/A | 366.1 | N/A |
| 296 | 5521.174696 | 33.2 | 27 | 1105.6 | 5 | 1105.6 | 5 | 366.1 | 1359.6 |
| 257 | 3342.25967 | 10.6 | 30 | 1115.1 | 3 | 1115.1 | 3 | 366.1 | 1341.6 |

Table 37 shows various parameters associated with the identification of the peptide and glycopeptides using LC and MRM-MS. The retention time (RT) represents the amount of time in minutes for the peptide elute from the chromatography column. The collision energy represents the energy applied to the peptide for creating fragments (i.e., product ions) such as, for example, in the 2^{nd} quadrupole of the triple quadrupole MS. The first precursor m/z represents a ratio value associated with an ionized form having a first precursor charge for the peptide or glycopeptide. Similarly, the second precursor m/z represents a ratio value associated with an ionized form having a second precursor charge for the peptide or glycopeptide. The first precursor ion is associated with a first product ion having a m/z ratio that was formed from a collision and the second precursor ion is associated with a second product ion having a m/z ratio that was formed from a collision. Under certain circumstances, the first precursor and the second precursor may be the same, but the associated first and second product m/z ratios are different.

To demonstrate the statistical significance in the peptide structure concentration difference between the healthy control and NSCLC populations (stages 1-4 population, stages 1-2 population, or stages 3-4 population), the fold changes, p-values, false discovery rates (FDR), and area under the curved (AUC) using one marker in the model are provided in Tables 38A and 38B. Fold-changes for individual peptides and glycopeptides, were calculated on normalized abundances of NSCLC stages 1-4 samples vs control (Table 38A). NSCLC stages 1-2 samples vs control (Table 38B), and NSCLC stages 3-4 vs control samples (Table 38B). For example, the fold change can be based the concentration of a biomarker from one of the NSCLC cohorts divided by the concentration of the same biomarker from the control cohort. In general, fold changes that are much higher or lower than unity are associated with a difference between the two cohorts. False discovery rate was calculated using the Benjamini-Hochberg method. Age- and sex-adjusted differential expression analysis for 596 normalized biomarkers were performed to evaluate statistically significant differential abundances using an FDR-adjusted q-value of 0.05 as a cutoff. Repeated five-fold cross-validated LASSO-regularized logistic regression was performed to create a multivariable classifier that predicts whether a serum sample belongs to the healthy or NSCLC cohort.

TABLE 38A
Differential expression analysis for healthy
control and NSCLC (stages 1-4) sample sets

| | Stages | | | Area under |
|---|---|---|---|---|
| | 1-4 / | | False | curve using |
| | Control | | Discovery | only one |
| | Fold | | Rate | marker in |
| SEQ ID NO | Change | p value | (FDR) | model |
| 271 | 1.272 | 4.42E−30 | 1.18E−27 | 0.8526 |
| 261 | 0.78 | 6.95E−28 | 1.23E−25 | 0.8732 |
| 282 | 2.583 | 1.07E−26 | 1.42E−24 | 0.8461 |
| 275 | 2.08 | 1.03E−25 | 1.09E−23 | 0.8434 |
| 263 | 1.267 | 3.59E−25 | 3.19E−23 | 0.8153 |
| 266 | 0.633 | 6.74E−25 | 5.12E−23 | 0.8364 |
| 262 | 0.757 | 8.08E−25 | 5.37E−23 | 0.8358 |
| 279 | 0.472 | 2.11E−24 | 1.25E−22 | 0.8253 |
| 277 | 0.526 | 6.60E−23 | 3.51E−21 | 0.8236 |
| 258 | 0.734 | 4.93E−22 | 2.38E−20 | 0.831 |
| 226 | 0.821 | 7.77E−22 | 3.44E−20 | 0.8372 |
| 259 | 0.772 | 6.78E−20 | 2.69E−18 | 0.793 |
| 284 | 0.758 | 7.09E−20 | 2.69E−18 | 0.8161 |
| 267 | 0.714 | 1.34E−19 | 4.74E−18 | 0.823 |
| 246 | 0.692 | 1.24E−18 | 4.12E−17 | 0.8777 |
| 260 | 0.65 | 1.32E−18 | 4.12E−17 | 0.8119 |
| 274 | 2.187 | 1.76E−18 | 5.20E−17 | 0.803 |
| 278 | 0.505 | 4.29E−18 | 1.20E−16 | 0.7883 |
| 294 | 1.956 | 5.84E−18 | 1.55E−16 | 0.8053 |
| 296 | 1.408 | 1.08E−17 | 2.74E−16 | 0.7992 |
| 227 | 1.337 | 1.55E−17 | 3.75E−16 | 0.7764 |
| 276 | 1.61 | 1.76E−17 | 4.07E−16 | 0.8271 |
| 286 | 0.777 | 3.65E−17 | 7.77E−16 | 0.8111 |
| 270 | 1.382 | 8.66E−17 | 1.77E−15 | 0.8192 |
| 268 | 0.729 | 1.05E−16 | 2.07E−15 | 0.7722 |
| 265 | 0.775 | 1.10E−16 | 2.09E−15 | 0.8127 |
| 295 | 0.905 | 3.08E−16 | 5.65E−15 | 0.785 |
| 281 | 1.487 | 3.26E−16 | 5.66E−15 | 0.7524 |
| 290 | 1.371 | 7.95E−16 | 1.32E−14 | 0.8152 |
| 273 | 1.218 | 2.95E−15 | 4.76E−14 | 0.7959 |
| 236 | 1.355 | 4.44E−15 | 6.75E−14 | 0.807 |
| 264 | 0.789 | 4.63E−15 | 6.84E−14 | 0.7997 |
| 292 | 1.214 | 4.90E−15 | 7.04E−14 | 0.793 |
| 287 | 1.376 | 9.02E−15 | 1.26E−13 | 0.8415 |
| 257 | 1.163 | 1.06E−14 | 1.43E−13 | 0.7496 |
| 283 | 1.257 | 1.50E−14 | 1.92E−13 | 0.7798 |
| 280 | 1.178 | 1.52E−14 | 1.92E−13 | 0.7922 |
| 272 | 1.199 | 3.13E−14 | 3.70E−13 | 0.7726 |
| 293 | 1.469 | 5.12E−14 | 5.92E−13 | 0.7975 |
| 289 | 0.88 | 5.47E−14 | 6.20E−13 | 0.7773 |
| 269 | 1.434 | 1.00E−13 | 1.11E−12 | 0.7993 |
| 244 | 1.359 | 2.37E−13 | 2.48E−12 | 0.8362 |
| 285 | 1.217 | 6.08E−13 | 6.22E−12 | 0.7774 |
| 291 | 1.106 | 1.52E−12 | 1.49E−11 | 0.8038 |
| 241 | 1.113 | 3.92E−12 | 3.73E−11 | 0.7083 |
| 288 | 0.853 | 5.09E−12 | 4.75E−11 | 0.8071 |
| 231 | 0.806 | 2.41E−11 | 2.00E−10 | 0.6848 |
| 242 | 1.32 | 1.40E−10 | 1.12E−09 | 0.7999 |
| 237 | 1.051 | 1.14E−09 | 7.81E−09 | 0.6865 |
| 229 | 1.36 | 1.72E−09 | 1.12E−08 | 0.7383 |
| 243 | 1.301 | 1.79E−09 | 1.15E−08 | 0.8077 |
| 245 | 0.636 | 2.61E−09 | 1.61E−08 | 0.7035 |
| 230 | 1.43 | 4.58E−09 | 2.68E−08 | 0.7325 |
| 240 | 1.492 | 7.60E−09 | 4.21E−08 | 0.7622 |
| 232 | 1.302 | 1.49E−08 | 7.78E−08 | 0.7192 |
| 251 | 1.271 | 1.11E−07 | 5.01E−07 | 0.6912 |
| 235 | 0.891 | 6.53E−06 | 2.17E−05 | 0.6638 |
| 256 | 0.944 | 8.00E−06 | 2.63E−05 | 0.6892 |
| 247 | 0.699 | 4.27E−03 | 8.42E−03 | 0.5962 |
| 238 | 0.904 | 1.68E−02 | 2.91E−02 | 0.5722 |
| 234 | 0.962 | 3.93E−02 | 6.28E−02 | 0.6331 |
| 248 | 0.97 | 1.11E−01 | 1.57E−01 | 0.6445 |
| 233 | 1.036 | 1.25E−01 | 1.74E−01 | 0.5753 |
| 250 | 0.97 | 1.32E−01 | 1.81E−01 | 0.5959 |
| 249 | 0.939 | 1.47E−01 | 1.97E−01 | 0.698 |
| 224 | 0.984 | 4.29E−01 | 5.03E−01 | 0.5457 |
| 228 | 1.019 | 4.58E−01 | 5.26E−01 | 0.5308 |
| 239 | 0.973 | 4.64E−01 | 5.32E−01 | 0.4884 |
| 225 | 0.989 | 7.30E−01 | 7.86E−01 | 0.5634 |

TABLE 38B
Differential expression analysis for healthy control
and NSCLC (stages 1-2 or stages 3-4) sample sets

| | Stages | | | Area under | Stages | | | Area under |
|---|---|---|---|---|---|---|---|---|
| | 1-2/ | | False | curve using | 3-4/ | | False | curve using |
| SEQ | Control | | Discovery | only one | Control | | Discovery | only one |
| ID | Fold | p | Rate | marker in | Fold | p | Rate | marker in |
| NO | Change | value | (FDR) | model | Change | value | (FDR) | model |
| 271 | 1.271 | 6.83E−23 | 1.82E−20 | 0.8433 | 1.263 | 1.97E−21 | 1.90E−19 | 0.8682 |
| 261 | 0.797 | 8.86E−20 | 1.18E−17 | 0.867 | 0.765 | 1.29E−22 | 3.44E−20 | 0.8809 |
| 282 | 2.179 | 1.04E−15 | 3.47E−14 | 0.8358 | 2.982 | 1.02E−21 | 1.80E−19 | 0.8616 |
| 275 | 1.911 | 1.15E−17 | 6.11E−16 | 0.8293 | 2.229 | 2.15E−21 | 1.90E−19 | 0.8599 |
| 263 | 1.268 | 5.01E−19 | 4.68E−17 | 0.8167 | 1.259 | 3.41E−15 | 1.55E−13 | 0.8117 |
| 266 | 0.648 | 1.23E−16 | 5.05E−15 | 0.8268 | 0.605 | 7.49E−21 | 5.70E−19 | 0.8458 |
| 262 | 0.777 | 6.05E−18 | 3.58E−16 | 0.8342 | 0.749 | 5.35E−18 | 3.37E−16 | 0.8371 |
| 279 | 0.537 | 2.66E−14 | 7.45E−13 | 0.8119 | 0.412 | 1.56E−21 | 1.90E−19 | 0.8463 |
| 277 | 0.534 | 6.24E−17 | 2.77E−15 | 0.8266 | 0.527 | 8.71E−15 | 3.09E−13 | 0.8197 |
| 258 | 0.712 | 5.16E−20 | 9.14E−18 | 0.8452 | 0.76 | 1.07E−12 | 2.22E−11 | 0.8077 |
| 226 | 0.81 | 1.79E−17 | 8.66E−16 | 0.8269 | 0.828 | 4.66E−15 | 1.77E−13 | 0.8537 |
| 259 | 0.746 | 5.28E−19 | 4.68E−17 | 0.8131 | 0.806 | 1.20E−09 | 1.39E−08 | 0.7618 |
| 284 | 0.754 | 9.75E−16 | 3.46E−14 | 0.809 | 0.772 | 8.44E−13 | 1.87E−11 | 0.8267 |
| 267 | 0.683 | 4.46E−18 | 2.97E−16 | 0.8484 | 0.764 | 2.51E−09 | 2.52E−08 | 0.7935 |
| 246 | 0.718 | 4.45E−12 | 8.01E−11 | 0.8659 | 0.688 | 3.80E−15 | 1.55E−13 | 0.8921 |
| 260 | 0.618 | 3.86E−18 | 2.93E−16 | 0.8314 | 0.683 | 2.37E−11 | 4.36E−10 | 0.7819 |
| 274 | 1.923 | 6.65E−12 | 1.04E−10 | 0.8041 | 2.477 | 1.24E−14 | 4.11E−13 | 0.7981 |
| 278 | 0.484 | 1.35E−15 | 4.22E−14 | 0.7875 | 0.545 | 1.93E−13 | 5.41E−12 | 0.7886 |
| 294 | 1.734 | 1.51E−11 | 2.06E−10 | 0.7822 | 2.153 | 1.49E−16 | 7.90E−15 | 0.8403 |
| 296 | 1.392 | 1.56E−13 | 3.99E−12 | 0.7885 | 1.401 | 4.25E−13 | 1.02E−11 | 0.8132 |
| 227 | 1.317 | 1.58E−13 | 3.99E−12 | 0.7688 | 1.361 | 1.93E−13 | 5.41E−12 | 0.7824 |
| 276 | 1.58 | 2.90E−12 | 5.92E−11 | 0.8198 | 1.658 | 3.66E−15 | 1.55E−13 | 0.8379 |
| 286 | 0.772 | 2.04E−14 | 6.02E−13 | 0.81 | 0.795 | 1.81E−10 | 2.60E−09 | 0.8113 |
| 270 | 1.356 | 1.02E−12 | 2.36E−11 | 0.8119 | 1.37 | 1.60E−10 | 2.36E−09 | 0.8246 |
| 268 | 0.727 | 1.60E−12 | 3.55E−11 | 0.7618 | 0.714 | 3.48E−13 | 8.83E−12 | 0.7921 |
| 265 | 0.765 | 5.20E−16 | 1.98E−14 | 0.8065 | 0.774 | 7.00E−12 | 1.33E−10 | 0.8215 |
| 295 | 0.914 | 4.54E−12 | 8.01E−11 | 0.7843 | 0.897 | 2.59E−13 | 6.88E−12 | 0.7846 |
| 281 | 1.416 | 8.53E−11 | 1.01E−09 | 0.7506 | 1.506 | 6.22E−12 | 1.23E−10 | 0.753 |
| 290 | 1.335 | 4.56E−10 | 5.05E−09 | 0.8026 | 1.428 | 5.70E−18 | 3.37E−16 | 0.8277 |
| 273 | 1.228 | 3.94E−12 | 7.49E−11 | 0.8036 | 1.198 | 7.56E−10 | 9.81E−09 | 0.7828 |
| 236 | 1.355 | 1.12E−11 | 1.62E−10 | 0.8085 | 1.34 | 2.67E−10 | 3.55E−09 | 0.8039 |
| 264 | 0.785 | 3.63E−12 | 7.15E−11 | 0.8092 | 0.817 | 1.39E−08 | 1.19E−07 | 0.7847 |
| 292 | 1.215 | 5.43E−12 | 8.83E−11 | 0.7836 | 1.217 | 1.03E−10 | 1.61E−09 | 0.803 |
| 287 | 1.309 | 1.50E−08 | 1.21E−07 | 0.827 | 1.386 | 1.17E−10 | 1.78E−09 | 0.8585 |
| 257 | 1.173 | 7.02E−12 | 1.07E−10 | 0.747 | 1.14 | 1.99E−09 | 2.10E−08 | 0.7518 |
| 283 | 1.253 | 5.01E−11 | 6.50E−10 | 0.7809 | 1.22 | 4.91E−09 | 4.75E−08 | 0.7804 |
| 280 | 1.147 | 8.45E−10 | 8.32E−09 | 0.7795 | 1.19 | 2.93E−11 | 5.20E−10 | 0.8105 |
| 272 | 1.169 | 1.43E−08 | 1.17E−07 | 0.76 | 1.214 | 4.88E−11 | 8.38E−10 | 0.7873 |
| 293 | 1.507 | 6.85E−13 | 1.66E−11 | 0.7938 | 1.462 | 1.03E−10 | 1.61E−09 | 0.7986 |
| 289 | 0.871 | 1.12E−11 | 1.62E−10 | 0.7886 | 0.885 | 1.64E−08 | 1.38E−07 | 0.7583 |
| 269 | 1.401 | 1.52E−09 | 1.44E−08 | 0.8016 | 1.438 | 8.33E−10 | 1.03E−08 | 0.7917 |
| 244 | 1.318 | 4.86E−08 | 3.40E−07 | 0.8403 | 1.29 | 1.77E−06 | 8.71E−06 | 0.8276 |
| 285 | 1.218 | 2.71E−09 | 2.36E−08 | 0.7877 | 1.186 | 1.32E−06 | 6.73E−06 | 0.7652 |
| 291 | 1.108 | 5.17E−09 | 4.31E−08 | 0.798 | 1.101 | 2.72E−07 | 1.62E−06 | 0.8099 |
| 241 | 1.116 | 6.99E−10 | 7.15E−09 | 0.7033 | 1.108 | 6.88E−08 | 4.81E−07 | 0.7162 |
| 288 | 0.868 | 6.76E−08 | 4.56E−07 | 0.7939 | 0.851 | 1.07E−09 | 1.27E−08 | 0.8236 |
| 231 | 0.803 | 7.81E−11 | 9.66E−10 | 0.7076 | 0.83 | 3.26E−07 | 1.86E−06 | 0.6661 |
| 242 | 1.304 | 2.16E−08 | 1.69E−07 | 0.7954 | 1.316 | 1.64E−07 | 1.01E−06 | 0.8024 |
| 237 | 1.045 | 2.96E−06 | 1.47E−05 | 0.6727 | 1.055 | 6.58E−07 | 3.55E−06 | 0.7045 |
| 229 | 1.308 | 6.03E−06 | 2.72E−05 | 0.7353 | 1.404 | 2.00E−08 | 1.63E−07 | 0.7423 |
| 243 | 1.284 | 2.22E−06 | 1.14E−05 | 0.8059 | 1.27 | 1.15E−05 | 4.73E−05 | 0.8066 |
| 245 | 0.562 | 8.36E−11 | 1.01E−09 | 0.7393 | 0.736 | 6.69E−04 | 1.88E−03 | 0.6516 |
| 230 | 1.544 | 2.66E−09 | 2.36E−08 | 0.7616 | 1.304 | 2.78E−06 | 1.31E−05 | 0.6961 |
| 240 | 1.571 | 5.18E−09 | 4.31E−08 | 0.7785 | 1.372 | 1.29E−04 | 4.44E−04 | 0.7377 |
| 232 | 1.346 | 2.93E−08 | 2.22E−07 | 0.7255 | 1.216 | 5.43E−05 | 2.05E−04 | 0.7145 |
| 251 | 1.224 | 9.88E−05 | 3.37E−04 | 0.667 | 1.302 | 9.05E−08 | 5.87E−07 | 0.7175 |
| 235 | 0.893 | 6.72E−05 | 2.40E−04 | 0.6475 | 0.887 | 2.20E−04 | 7.09E−04 | 0.6825 |
| 256 | 0.946 | 1.39E−04 | 4.59E−04 | 0.6747 | 0.941 | 7.90E−05 | 2.86E−04 | 0.7101 |
| 247 | 0.687 | 1.10E−02 | 2.28E−02 | 0.6102 | 0.651 | 8.10E−03 | 1.62E−02 | 0.5773 |
| 238 | 0.947 | 2.66E−01 | 3.63E−01 | 0.5368 | 0.858 | 3.83E−03 | 9.01E−03 | 0.617 |
| 234 | 0.958 | 4.13E−02 | 7.27E−02 | 0.5949 | 0.958 | 6.65E−02 | 1.02E−01 | 0.6803 |
| 248 | 0.976 | 2.62E−01 | 3.58E−01 | 0.6358 | 0.951 | 3.40E−02 | 5.70E−02 | 0.6619 |
| 233 | 1.061 | 2.71E−02 | 5.13E−02 | 0.6076 | 1.006 | 8.37E−01 | 8.71E−01 | 0.5361 |
| 250 | 0.958 | 6.97E−02 | 1.16E−01 | 0.5961 | 0.985 | 5.28E−01 | 6.03E−01 | 0.6033 |
| 249 | 1.005 | 9.18E−01 | 9.34E−01 | 0.6674 | 0.907 | 6.60E−02 | 1.02E−01 | 0.7415 |
| 224 | 0.979 | 3.48E−01 | 4.43E−01 | 0.5375 | 0.984 | 4.76E−01 | 5.56E−01 | 0.5517 |
| 228 | 1.05 | 9.53E−02 | 1.52E−01 | 0.5374 | 0.992 | 7.79E−01 | 8.17E−01 | 0.4827 |
| 239 | 0.999 | 9.80E−01 | 9.82E−01 | 0.4995 | 0.966 | 4.20E−01 | 4.95E−01 | 0.5282 |
| 225 | 0.954 | 2.14E−01 | 3.02E−01 | 0.5789 | 1.024 | 5.51E−01 | 6.24E−01 | 0.5357 |

### Example 6. Training and Validation of Diagnostic Models

The quantified concentrations of various peptide structures identified in Table 35 (e.g., SEQ ID NOs: 224-296) across the entire sample set were used to train a regularized regression model (e.g., LASSO regression model) to generate a disease indicator for a subject. The disease indicator was generated as a score (e.g., a probability score), wherein the range in which the score falls enables diagnosis or classification as a healthy state or a NSCLC state. The same markers were used to train a regularized regression model (e.g., LASSO regression model). Coefficients for the regularized regression model (e.g., LASSO regression model) are provided in Table 39.

TABLE 39
Coefficients for each marker used in the regularized
regression model (e.g., LASSO regression model)

| | Coefficients for | Coefficients for | Coefficients for |
|---|---|---|---|
| | model predicting | model predicting | model predicting |
| | probability of | probability of | probability of |
| | having NSCLC | having NSCLC | having NSCLC |
| | based on control | based on control | based on control |
| SEQ ID NO | vs stages 1-4 | vs stages 1-2 | vs stages 3-4 |
| (Intercept) | 0.75719123 | −0.0340956 | −0.4020039 |
| 224 | N/A | 0.17004465 | N/A |
| 225 | N/A | N/A | 0.053271 |
| 226 | N/A | N/A | −0.0169616 |
| 227 | N/A | 0.06704283 | N/A |
| 228 | N/A | N/A | 0.01076784 |
| 229 | 0.29409065 | N/A | 0.33640677 |
| 230 | N/A | N/A | 0.02291029 |
| 231 | −0.0263181 | N/A | N/A |
| 232 | 0.08908578 | N/A | N/A |
| 233 | 0.19048452 | 0.10331115 | 0.05524534 |
| 234 | −0.089109 | N/A | −0.0256861 |
| 235 | N/A | N/A | −0.1392993 |
| 236 | 0.05759125 | N/A | 0.15024667 |
| 237 | N/A | N/A | 0.06035004 |
| 238 | N/A | 0.09845643 | N/A |
| 239 | 0.40050612 | N/A | 0.28329752 |
| 240 | N/A | 0.25400149 | N/A |
| 241 | 0.20270253 | 0.18259914 | 0.00158212 |
| 242 | N/A | 0.16515737 | 0.05928281 |
| 243 | N/A | N/A | 0.08498688 |
| 244 | 0.41864233 | 0.36128256 | 0.39412099 |
| 245 | −0.0374678 | N/A | N/A |
| 246 | N/A | N/A | −0.0450124 |
| 247 | −0.1605237 | −0.0222012 | N/A |
| 248 | −0.055033 | −0.0309227 | N/A |
| 249 | −0.0577257 | N/A | N/A |
| 250 | −0.13873 | −0.0479855 | N/A |
| 251 | −0.0326004 | −0.171405 | N/A |
| 252 | 0.12386169 | 0.01319359 | N/A |
| 253 | −0.1090814 | −0.081731 | N/A |
| 254 | 0.00820188 | 0.17822201 | 0.10494401 |
| 255 | −0.3321263 | −0.3515829 | −0.1436638 |
| 256 | N/A | N/A | −0.192089 |
| 257 | N/A | N/A | 0.09775612 |

Using the values of Table 39, a probability can be determined by summing together the product of the concentration of each biomarker in the sample and the respective coefficient (of one column) and then adding the summation and the intercept to yield the logit of a probability score. For example, the logit of the probability, to which the inverse logit function can be applied, is equal to:

$${\sum }_{i = 27}^{i = 60} [ ( {Concentration}_{SEQ ID NO : i} ) \times ( {Coefficient}_{SEQ ID NO : i} ) ] + Intercept$$

With respect to the above equations and Table 39, coefficients that were N/A could be construed as zero. Using a FDR<0.05 in comparing NSCLC and healthy control samples, 432 biomarkers with significant abundance differences were identified. Using 70% of the complete cohort (balanced by case/control membership, NSCLC stage, sex, and age quartile) as a training set, a total of 375 glycopeptide and non-glycosylated peptide biomarker features were selected that remained differentially expressed at FDR-adjusted q-value ≤0.05 as input into a LASSO-regularized multivariable classifier. This resulted in a 19-biomarker model (Table 40) exhibiting an accuracy of 94.8% (96.9% sensitivity, 91.2% specificity) and AUC of 0.989 in predicting NSCLC for all stages (i.e., stages 1-4). This classifier was validated in an independent test set including the remaining 30% of subjects, yielding an accuracy of 94.5% (95.5% sensitivity, 93.0% specificity) and AUC of 0.975. Sensitivity in the test set was 100%/96%/99%/96%/94%/100%, for NSCLC stages 0, 1, 2, 3, 4, and “unknown”, respectively. It should be noted that stage 0 is a cohort where a carcinoma is in situ and is considered the earliest stage at which lung cancer can be detected. The stage “unknown” refers to samples that have NSCLC, but the information regarding stage is unknown.

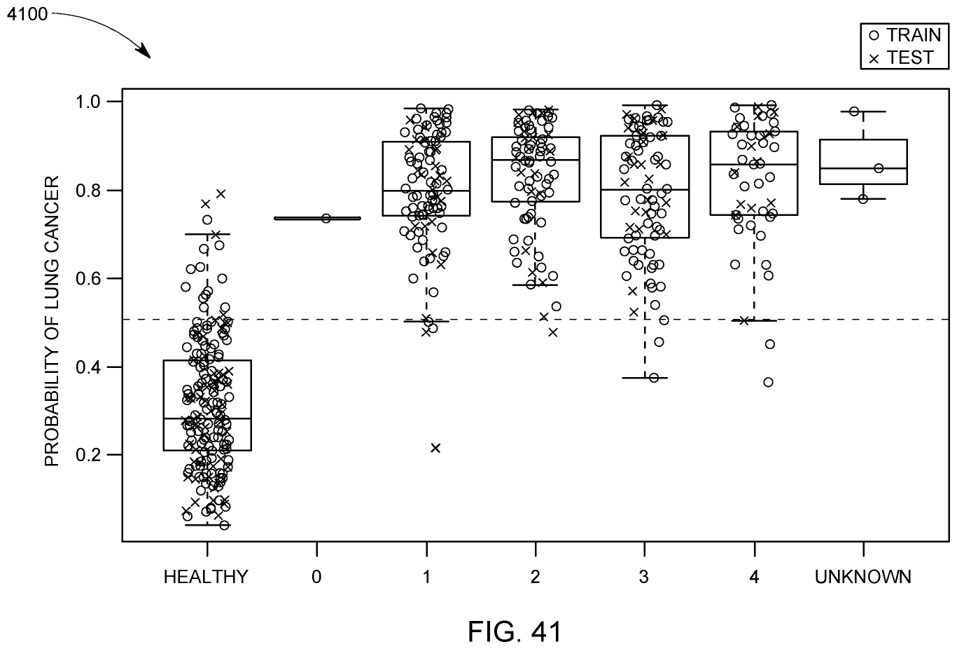

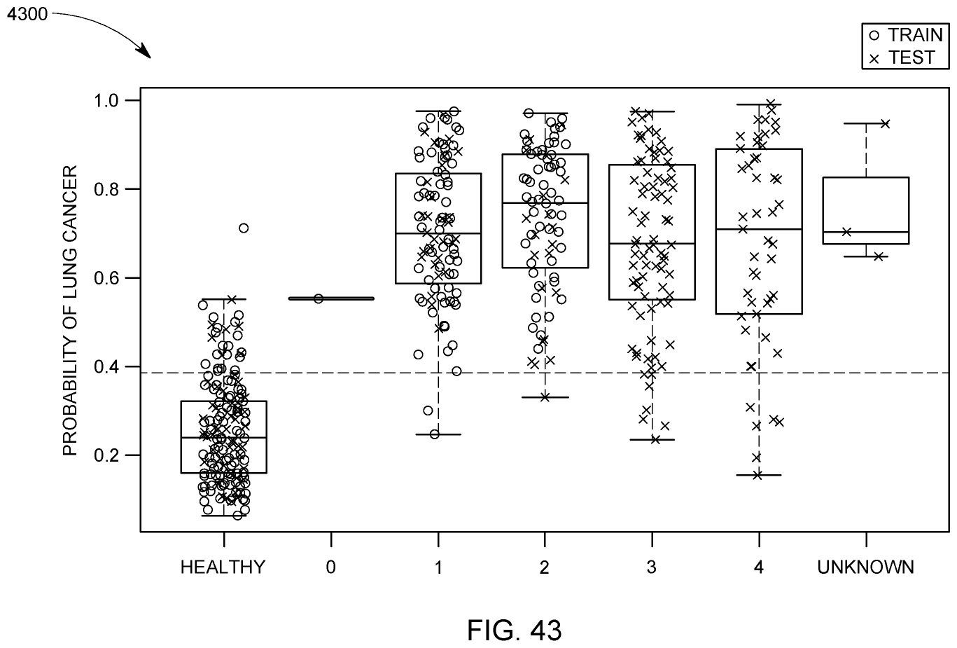

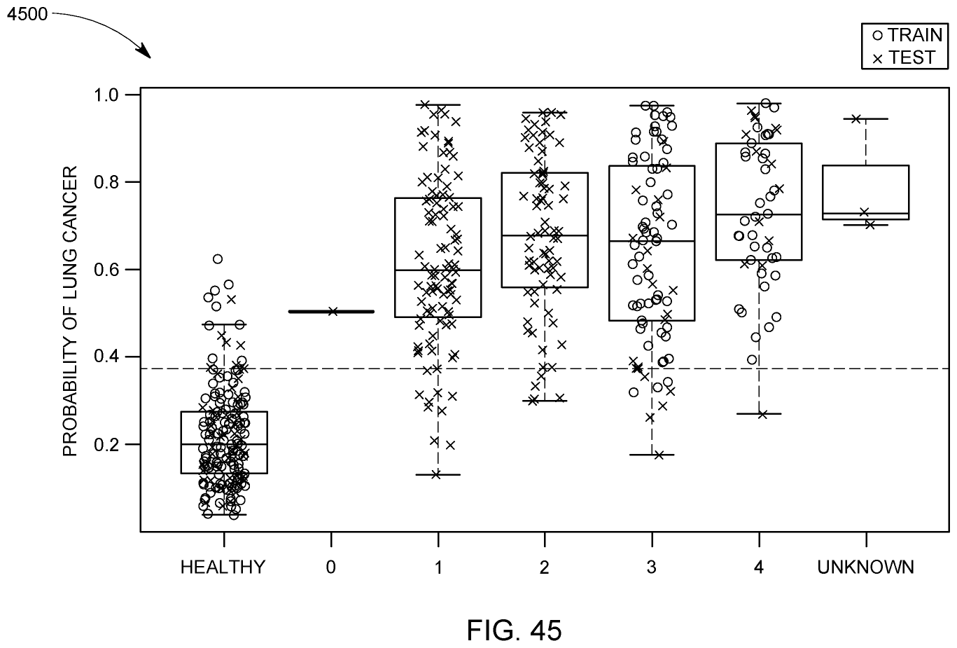

FIG. 41 is a model performance plot 1200 illustrating the predictive performance of a regularized regression model (e.g., LASSO regression model) trained and evaluated on the training data set and test data set splits for all stages of NSCLC (stages 1-4). FIG. 43 is a model performance plot 1400 illustrating the predictive performance of the regularized regression model (e.g., LASSO regression model) trained and evaluated on the training data set and test data set splits for early-stage NSCLC (stages 1-2). FIG. 45 is a model performance plot 1600 illustrating the predictive performance of the regularized regression model (e.g., LASSO regression model) trained and evaluated on the training data set and test data set splits for late-stage NSCLC (stages 3-4).

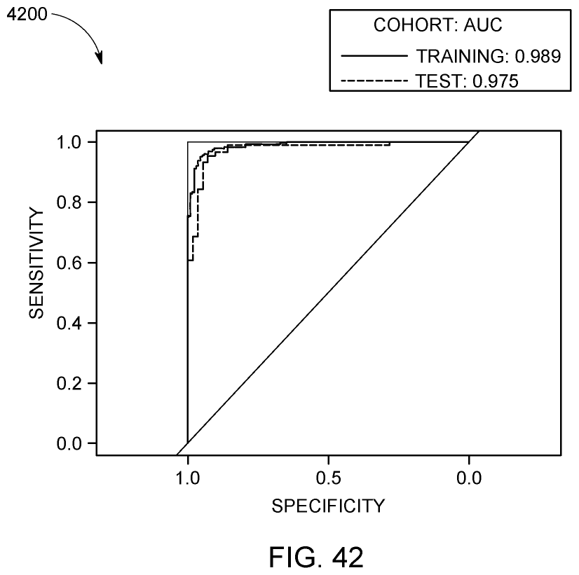

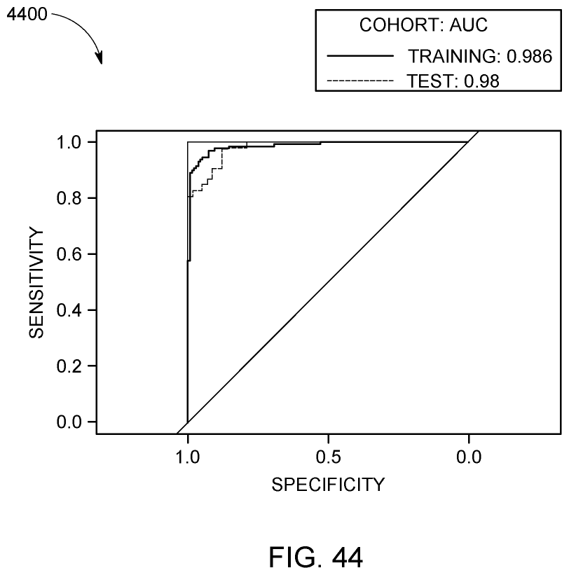

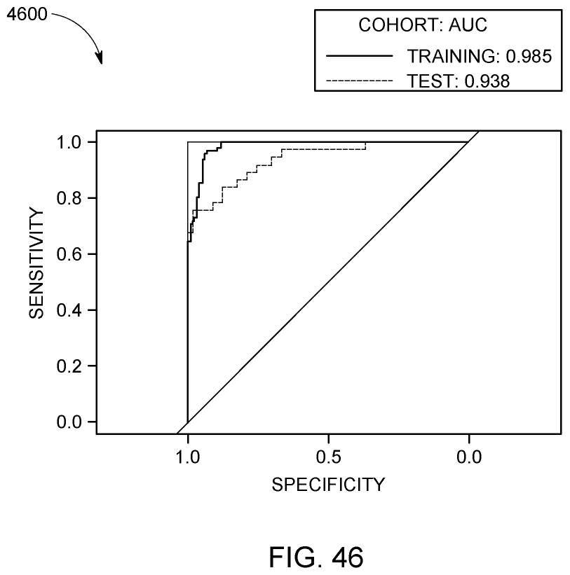

FIG. 42 illustrates a receiver-operating-characteristic (ROC) curve/area under curve (AUC) plot 1300, depicting that the regularized regression model (e.g., LASSO regression model) trained and evaluated on the training data set and test data set splits for all stages of NSCLC (stages 1-4) exhibited an accuracy of 94.8% and 94.5% on the training data set and test data set, respectively, and AUC of 0.98 in distinguishing between healthy and NSCLC individuals. FIG. 44 illustrates a receiver-operating-characteristic (ROC) curve/area under curve (AUC) plot 1500, depicting that the regularized regression model (e.g., LASSO regression model) trained and evaluated on the training data set and test data set splits for early-stage NSCLC (stages 1-2) exhibited an accuracy of 91.7% and 92.7% on the training data set and test data set, respectively, and AUC of 0.98 in distinguishing between healthy and NSCLC individuals. FIG. 46 illustrates a receiver-operating-characteristic (ROC) curve/area under curve (AUC) plot 1700, depicting that the regularized regression model (e.g., LASSO regression model) trained and evaluated on the training data set and test data set splits for late-stage NSCLC (stages 3-4) exhibited an accuracy of 94.8% and 86.2% on the training data set and test data set, respectively, and AUC of 0.98 in distinguishing between healthy and NSCLC individuals.

TABLE 40
Peptide and glycopeptide structures where
coefficients were calculated for a model predicting probability
of having NSCLC based on control vs stages 1-4

| | | | Glycan | Glycan | |
|---|---|---|---|---|---|
| | | | Linking | Linking | |
| | | | Site | Site | |
| SEQ | Peptide | | Pos. in | Pos. in | Glycan |
| ID | Structure | Peptide | Peptide | Protein | Structure |
| NO | (PS) NAME | Sequence | Sequence | Sequence | GL NO. |
| 229 | AACT_271_6513 | YTGNASAL | 4 | 271 | 6513 |
| | | FILPDQDK | | | |
| 231 | C1S_174_5402 | NCGVNCSG | 5 | 174 | 5402 |
| | | DVFTALIG | | | |
| | | EIASPNYP | | | |
| | | KPYPENSR | | | |
| 232 | CFAI_70_5402 | NGTAVCAT | 1 | 70 | 5402 |
| | | NR | | | |
| 233 | CO5_741_5401 | ANISHK | 2 | 741 | 5401 |
| 234 | FETUA_176_5402 | AALAAFNA | 11 | 176 | 5402 |
| | | QNNGSNFQ | | | |
| | | LEEISR | | | |
| 236 | HEMO_187_6513 | SWPAVGNC | 7 | 187 | 6513 |
| | | SSALR | | | |
| 239 | HPT_184_6502 | MVSHHNLT | 6 | 184 | 6502 |
| | | TGATLINE | | | |
| | | QWLLTTAK | | | |
| 241 | IC1_238_5402 | DTFVNASR | 5 | 238 | 5402 |
| 244 | IGG1_297_3510 | EEQYNSTYR | 5 | 180 | 3510 |
| 245 | IGG1_297_4500 | EEQYNSTYR | 5 | 180 | 4500 |
| 247 | IGG2_297_3500 | EEQFNSTFR | 5 | 175 | 3500 |
| 248 | IGG2_297_4410 | EEQFNSTFR | 5 | 175 | 4410 |
| 249 | IGG2_297_5410 | EEQFNSTFR | 5 | 175 | 5410 |
| 250 | KLKB1 | IYSGILNL | N/A | N/A | Non- |
| | | SDITK | | | Glycosylated |
| 251 | KLKB1_494_5410 | LQAPLNYT | 6 | 494 | 5410 |
| | | EFQKPICL | | | |
| | | PSK | | | |
| 252 | IGG3 | TPEVTCVV | N/A | N/A | Non- |
| | | VDVSHEDP | | | Glycosylated |
| | | EVQFK | | | |
| 253 | APOM | AFLLTPR | N/A | N/A | Non- |
| | | | | | Glycosylated |
| 254 | B2M | VNHVTLSQ | N/A | N/A | Non- |
| | | PK | | | Glycosylated |
| 255 | SEPP1 | VSLATVDK | N/A | N/A | Non- |
| | | | | | Glycosylated |

This result demonstrates that the identified peptide structures in Table 35 and a trained model using the peptide structures can be used to diagnose NSCLC.

### Example 7: Glycoproteomics as Liquid Biopsy-Based Predictor of Checkpoint-Inhibitor Treatment Response in Patients with Metastatic Malignant Melanoma

Protein glycosylation is one of the most abundant and most complex form of post-translational protein modification. Glycosylation affects protein structure, conformation, and function. The elucidation of the potential role of differential protein glycosylation as biomarkers has so far been limited by the technical complexity of generating and interpreting this information. A novel, powerful platform has been recently established that combines ultra-high-performance liquid chromatography coupled to triple quadrupole mass spectrometry with a proprietary machine-learning and neural-network-based data processing engine that allows for high-throughput, highly scalable interrogation of the glycoproteome. This study assessed whether glycoproteomic biomarkers and signatures can predict which patients with metastatic malignant melanoma would respond to PD1/PDL1 checkpoint inhibitors.

Methods: this platform we interrogated 413 individual glycopeptide (GP) signatures derived from 69 abundant serum proteins in pretreatment blood samples from a cohort of 36 individuals (11 females, 25 males, age range 28 to 90 years) with metastatic malignant melanoma treated either with nivolumab plus ipilimumab (12 patients) or pembrolizumab (24 patients). Plasma samples were taken prior to beginning treatment, stored at −80C, and run through InterVenn's targeted MRM panel.

The individual glycopeptide expression levels were associated with time from treatment initiation to progression/metastasis (progression-free survival, PFS) or death (overall survival, OS) in the patient cohorts.

In addition to assessing individual biomarker associations, multivariable models were built to predict PFS (Melanoma). The multivariate models were built by selecting a small subset of glycopeptides for modeling, proceeding to build a model with n-1 patients, predicting a survival score on the one holdout patient, and iterating over all patients as individual holdouts, to generate unbiased prediction scores for everyone (a leave-one-out cross-validation approach, LOOCV). The resulting scores were dichotomized at a cutoff which optimizes Harrell's C-index, and Kaplan-Meier (KM) curves were plotted.

Specifically, progression-free survival (PFS) data with follow-up of up to 3.7 years (median: 0.8 years) were used as clinical endpoint phenotype against which the predictive power of differential abundance of GPs was assessed. PFS data were analyzed using Cox Proportional Hazards models. Kaplan Meier curves were generated for GP markers that showed statistically significant differential abundances using a false discovery rate (FDR)—adjusted p-value of ≤0.1 as a cutoff. Hazard Ratio (HR) for PFS was calculated from a Cox Proportional Hazards model, representing the multiplicative increase in odds of progression for each increase of the biomarker by 1 unit. The p-value associated with the HR was analyzed, where p<0.01 was considered significant. The interaction p-value, the p-value associated with the biomarker x treatment interaction, was also analyzed, where significance indicates potential for use in treatment selection.

Further, as part of this example, an interrogation of 526 glycopeptide (GP) signatures derived from 75 serum proteins in pretreatment blood samples from a cohort of 205 individuals (66 females, 139 males, age range 24 to 97 years) with metastatic malignant melanoma treated either with nivolumab (N) with or without ipilimumab (I, 95 patients) or pembrolizumab (P, 110 patients) immune-checkpoint inhibitor (ICI) therapy.

In certain embodiments, FIGS. 73A and 73B illustrate the KM curves for a multivariable model, including the training phases and validation phases, respectively. Hazard ratios and p-values on the plots are representative of the high/low split at the risk score cut-off determined by optimizing for sensitivity for non-response. Study 1 KM curve of FIG. 73B labeled “Validation” contains patients from the validation and test data sets. In one example, the optimal model includes 6 biomarkers and a cutoff was selected in the validation set to optimize for sensitivity to response (e.g., test set 720 day performance: sensitivity=99.5%, specificity=25.6%) metrics/curves shown exclude Indeterminate calls (10% of patient set).

Results: 27 GPs with abundance differences at FDR p<0.1 were identified, and among them 8 markers at p<0.001. Using the latter 8 markers, a multivariable model for PFS was created by generating leave-one-out cross-validation (LOOCV) scores and determining an optimized cutoff value for these scores using Harrel's concordance index. Dichotomizing the LOOCV scores using this cutoff value demonstrated the model to yield a hazard ratio of 9.2 at a p-value of 10⁵ for separating treatment responders and non-responders (70% vs. 0% PFS, respectively, at 18 months based on LOOCV score above/below cutoff), as compared to a hazard ratio of 1.5, p=0.5 for PDL1 expression. FIG. 1 shows a Kaplan-Meier curve of patients with metastatic melanoma treated either with a combination of ipilimumab and nivolumab or pembrolizumab alone, where progression-free survival (PFS) was 61% at 2.7 years in the Low Score group (black) as compared to PFS of 50% at 0.10 years in the High Score group (blue).

In an optimized assay containing 27 glycopeptides and 20 non-glycosylated peptides, we identified 14 GPs with abundance differences at FDR q≤0.05 with regard to PFS. Using 40% of the cohort as a training set and selecting 12 glycopeptide and non-glycosylated peptide biomarker features of the 47 total by LASSO shrinkage, we created a multivariable-model-based classifier for PFS that yielded a hazard ratio (HR) for prediction of likely ICI benefit of 7.5 at p<0.0001. This classifier was validated in the test set comprised of the held-out 60% of patients, yielding a HR of 4.7 at a similar p-value for separating patients likely benefiting from either single or combination ICI therapy and those likely not benefiting (50% PFS of 18 months vs. 3 months based on classifier score above/below cutoff). This classifier has a sensitivity of >99% to predict likely ICI benefit, while still performing at a specificity of 26%, thus helping to safely reduce ultimately unnecessary and non-beneficial exposure to these agents of one in four who otherwise would unnecessarily be exposed to them.

Conclusions: Our results indicate that glycoproteomics holds a strong promise as a response predictor to checkpoint inhibitor treatment that appears to significantly outperform other currently pursued biomarker approaches in this context.

### Example 8: Blood-Based Glycoprotein Signatures in Patients with Advanced Non-Small-Cell Lung Carcinoma (NSCLC) Receiving First-Line Immune Checkpoint Blockade

Background: Immune checkpoint blockade is an integral component of first-line therapy for most patients with advanced non-small cell lung cancer (NSCLC), however individual patient outcomes are highly variable and improved biomarkers are needed. Protein glycosylation is an emerging mechanism of immune evasion in cancer. Blood-based glycopeptide signatures were examined in a cohort of advanced NSCLC patients treated with first-line immune checkpoint blockade. This study assessed whether glycoproteomic biomarkers and signatures can predict which patients with NSCLC would respond to PD1/PDL1 checkpoint inhibitors.

Methods: In two independent studies, whether glycoproteomic biomarkers and signatures may predict which patients would respond to checkpoint inhibitor therapies was determined. For example, Study 1 included of n=205 patients with metastatic melanoma seen at Massachusetts General Hospital (MGH), treated either with Ipilimumab+Nivolumab (n=95) or Pembrolizumab (n=110). Plasma samples were taken prior to beginning treatment, stored at −80C, and inputted to a targeted multiple reaction monitoring (MRM) panel. Study 2 included n=125 patients with metastatic non-small-cell lung cancer sourced from Tempus and treated with Pembrolizumab. Serum samples were taken prior to beginning treatment, stored at −80C, and inputted to the targeted MRM panel. In both Study 1 and Study 2, individual glycopeptide expression levels were associated with time from treatment initiation to progression-free survival (PFS) (e.g., progression/metastasis) or overall survival (OS) in the patient cohorts.

In addition to assessing individual biomarker associations, multivariable models were built to predict OS (NSCLC) and PFS (Melanoma). The multivariable models were built to predict OS (NSCLC) and PFS (Melanoma) by selecting a small subset of glycopeptides through 5-fold repeated cross-validated LASSO regularization, proceeding to build a model with 40% of patients (allocated via balanced stratification on sex, age quartile, PFS/OS event), tuning hyperparameters in LASSO model in another 30% of patients, and predicting a survival score on the remaining 30% of holdout patients (to generate unbiased prediction scores). The resulting prediction scores were dichotomized at a cutoff which optimizes Harrell's C-index, and Kaplan-Meier (KM) curves were plotted final models for products were optimized for sensitivity for non-response. For example, in certain embodiments, FIGS. 74A and 74B illustrate the KM curves for a multivariable model, including the training phases and validation phases, respectively. Hazard ratios and p-values on the plots are representative of the high/low split at the risk score cut-off determined by optimizing for sensitivity for non-response. Study 2 KM curve of FIG. 74B labeled “Validation” contains patients only from the independent/unseen test set since there was no validation set. In one example, the optimal model includes 6 biomarkers and a cutoff was selected in the validation set to optimize for sensitivity to response (e.g., test set 720 day performance: sensitivity=99.5%, specificity=25.6%) metrics/curves shown exclude Indeterminate calls (10% of patient set).

Results: 30 GPs with abundance differences using a False Discovery Rate (FDR) threshold of 0.05 were identified. Using the 5 most predictive GP markers, a multivariable model for OS was created by generating leave-one-out cross-validation (LOOCV) scores and determining an optimized cutoff value of −0.83 (range: −2.2-3.4) for these scores using Harrell's concordance index. The median overall survival was 2.8 years for patients (n=14) whose GP classifier value was above the cutoff and 0.8 years for patients (n=32) whose GP classifier value was below the cutoff (HR 7.4, 95% CI 1.7-32.1, p=0.007). The model's performance was not affected by sex, age, or treatment regimen.

Conclusions: Blood-based glycopeptide signatures may represent novel, non-invasive biomarkers of clinical outcome to first-line immune checkpoint blockade in advanced NSCLC. These findings may be validated in larger cohorts and applied in clinical decision-making.

### Example 9: Blood-Based Glycoprotein Signatures in Patients with Unresectable Non-Small-Cell Lung Carcinoma (NSCLC) Treated with Immune Checkpoint Inhibitors

Serum samples were taken pre-treatment from a cohort of 62 patients from Northwestern Memorial Hospital with unresectable NSCLC. The samples were processed in accordance with the combined LC-MS methods described herein followed by AI-driven data analysis, which yielded 20 glycopeptide biomarkers strongly associated with progression of disease (Tables 58A.1 and 58A.2) and 20 glycopeptide biomarkers strongly associated with death (Tables 58B.1 and 58B.2) in this cohort.

### Example 10: Blood-Based Glycoprotein Signatures in Patients with Advanced Non-Small-Cell Lung Carcinoma (NSCLC) Receiving First-Line Immune Checkpoint Blockad

### BACKGROUND

Immune checkpoint blockade is an integral component of first-line therapy for most patients with advanced non-small-cell lung cancer (NSCLC), however individual patient outcomes are highly variable and improved biomarkers are needed. Protein glycosylation is an emerging mechanism of immune evasion in cancer. Protein glycosylation is the most abundant and complex form of post-translational protein modification. Glycosylation profoundly affects protein structure, conformation, and function. The elucidation of the potential role of differential protein glycosylation as biomarkers has been limited by the technical complexity of generating and interpreting this information. Blood-based glycopeptide signatures were examined in a cohort of advanced NSCLC patients treated with first-line immune checkpoint blockade. This study assessed whether glycoproteomic biomarkers and signatures can predict which patients with NSCLC would respond to pembrolizumab.

Methods: A study was conducted to determine whether glycoproteomic biomarkers and signatures may predict which patients would respond to checkpoint inhibitor therapies. The study included n=125 patients (54 females, 71 males, age range 60 to 75 years) with metastatic non-small-cell lung cancer (NSCLC) sourced from Tempus and treated with Pembrolizumab. Inclusion criteria were as follows: a diagnosis of unresectable stage 3 or 4 NSCLC, treatment with pembrolizumab monotherapy (27 patients), or treatment with combination pembrolizumab-chemotherapy (98 patients). Serum samples were taken prior to beginning treatment, stored at −80C, and inputted to the targeted MRM panel. Individual glycopeptide expression levels were associated with time from treatment initiation to overall survival (OS) in the patient cohorts.

In addition to assessing individual biomarker associations, a multivariable model was built to predict OS. The multivariable model was built to predict OS by selecting a small subset of glycopeptides through 5-fold repeated cross-validated elastic net and LASSO regularization, proceeding to build a generalized additive model (GAM) with 40% of patients (allocated via balanced stratification on sex, age quartile, OS event), tuning hyperparameters of the GAM in another 30% of patients, and predicting a survival score on the remaining 30% of holdout patients (to generate unbiased prediction scores). The resulting prediction scores were dichotomized at a cutoff which optimizes Harrell's C-index, and Kaplan-Meier (KM) curves were plotted final models for products were optimized for sensitivity for non-response. For example, in certain embodiments, FIGS. 147A and 147B illustrate the KM curves for a multivariable model, including the training phases and validation phases, respectively. Hazard ratios and p-values on the plots are representative of the high/low split at the risk score cut-off determined by optimizing for sensitivity for non-response. The KM curve of FIG. 147B labeled “Validation” contains patients only from the independent/unseen test set since there was no validation set. In one example, the optimal model includes 7 biomarkers and a cutoff was selected in the validation set to optimize for hazard ratio metrics. The curves shown exclude Indeterminate calls (10% of patient set). All analysis was conducted using the R 4.2.1 software package.

Results: 23 peptides and 49 glycopeptides with abundance differences using a False Discovery Rate (FDR) threshold of 0.05 were identified. Using the 7 most predictive GP markers, a multivariable generalized additive model for OS was created by generating risk scores on the training set and determining an optimized cutoff value of −0.50 (range: −1.62-0.66) for these scores using Harrell's concordance index. Any sample with a generated risk score higher than the selected cutoff value was predicted to be part of the “unlikely to benefit from treatment” cohort and any sample with a generated risk score lower than the selected cutoff value was predicted to be part of the “likely to benefit from treatment” cohort. Of the 7 selected markers, 5 of the markers were adjusted for their non-linear relationships with the outcome during model training on the training set. These 5 markers correspond to SEQ ID NOs: 1003, 1004, and 1006-1008 and were adjusted for non-linearity using smoothing splines with 9 spline basis functions for each non-linear feature. The 2 markers corresponding to SEQ ID NOs: 1002 and 1005 were treated linearly. The hyperparameters for the optimized model are provided in Table 83. The median overall survival was 5.8 months for patients (n=8) whose GP classifier value was above the cutoff and 23.16 months for patients (n=29) whose GP classifier value was below the cutoff (HR 3.858, 95% CI 1.86-15.83, p=0.005). The model's performance was not affected by sex, age, or treatment regimen. In some embodiments, a hazard ratio of >3 indicates that the model has a good fit and strong performance comparing the “likely to benefit from treatment” cohort versus the “unlikely to benefit from treatment” cohort. It should be noted that the hazard ratios (HR) are similar in magnitude for both the training and testing datasets (FIGS. 147A and 147B, respectively), which implies that the model was not overfit.

TABLE 83
Hyperparameters for generalized additive model

| SEQ ID | | |
|---|---|---|
| NO. | Spline No. | Coefficient |
| 1002 | N/A (linear term) | −4.0391 |
| 1003 | 1 | −3.7E−07 |
| 1003 | 2 | 2.53E−07 |
| 1003 | 3 | −4.3E−07 |
| 1003 | 4 | 7.79E−07 |
| 1003 | 5 | −3.3E−07 |
| 1003 | 6 | −7.1E−07 |
| 1003 | 7 | 3.85E−07 |
| 1003 | 8 | −4.5E−06 |
| 1003 | 9 | 0.594069 |
| 1004 | 1 | 2.16E−06 |
| 1004 | 2 | 2.85E−06 |
| 1004 | 3 | 2.54E−06 |
| 1004 | 4 | 7.21E−06 |
| 1004 | 5 | −2.6E−06 |
| 1004 | 6 | −5.7E−06 |
| 1004 | 7 | 2.14E−06 |
| 1004 | 8 | −4.1E−05 |
| 1004 | 9 | −1.02837 |
| 1005 | N/A (linear term) | 0.156552 |
| 1006 | 1 | −0.00058 |
| 1006 | 2 | 0.002245 |
| 1006 | 3 | 0.000918 |
| 1006 | 4 | 0.003106 |
| 1006 | 5 | −0.00156 |
| 1006 | 6 | −0.0027 |
| 1006 | 7 | 0.0017 |
| 1006 | 8 | −0.01733 |
| 1006 | 9 | 0.956383 |
| 1007 | 1 | 1.13E−06 |
| 1007 | 2 | −1.3E−06 |
| 1007 | 3 | −6.4E−08 |
| 1007 | 4 | 1.23E−06 |
| 1007 | 5 | 4.64E−08 |
| 1007 | 6 | 1.2E−06 |
| 1007 | 7 | −8.5E−08 |
| 1007 | 8 | −7.6E−06 |
| 1007 | 9 | 0.165953 |
| 1008 | 1 | 3.37E−06 |
| 1008 | 2 | 3.24E−06 |
| 1008 | 3 | −6.2E−07 |
| 1008 | 4 | −1.5E−06 |
| 1008 | 5 | −4E−08 |
| 1008 | 6 | −1.3E−06 |
| 1008 | 7 | 2.95E−08 |
| 1008 | 8 | −6.4E−06 |
| 1008 | 9 | −0.03973 |

Conclusions: The glycoproteomic-based classifier described here predicts with high sensitivity which patients are likely to benefit from ICI therapy. In addition to potentially reducing the use of ICIs in a safe manner in patients who would be unnecessarily subjected to possible adverse drug reactions, this classifier simultaneously has the potential of reducing the burden of health care expenditures. The presented results indicate that glycoproteomics holds a strong promise as a predictor for ICI treatment benefit that appears to significantly outperform other currently pursued biomarker approaches. Blood-based glycopeptide signatures may represent novel, non-invasive biomarkers of clinical outcome to first-line immune checkpoint blockade in advanced NSCLC. These findings may be validated in larger cohorts and applied in clinical decision-making as contemplated above.

### Example 11: Diagnostic Testing to Indicate Lack of Benefit from ICI Therapy for Subjects with Malignant Melanoma Before and After ICI Therapy

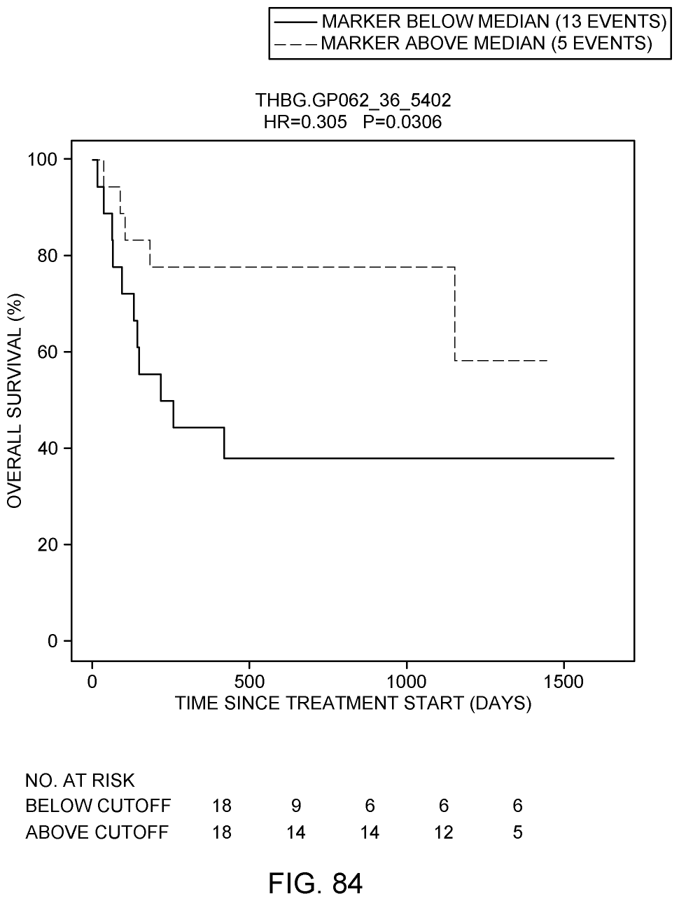

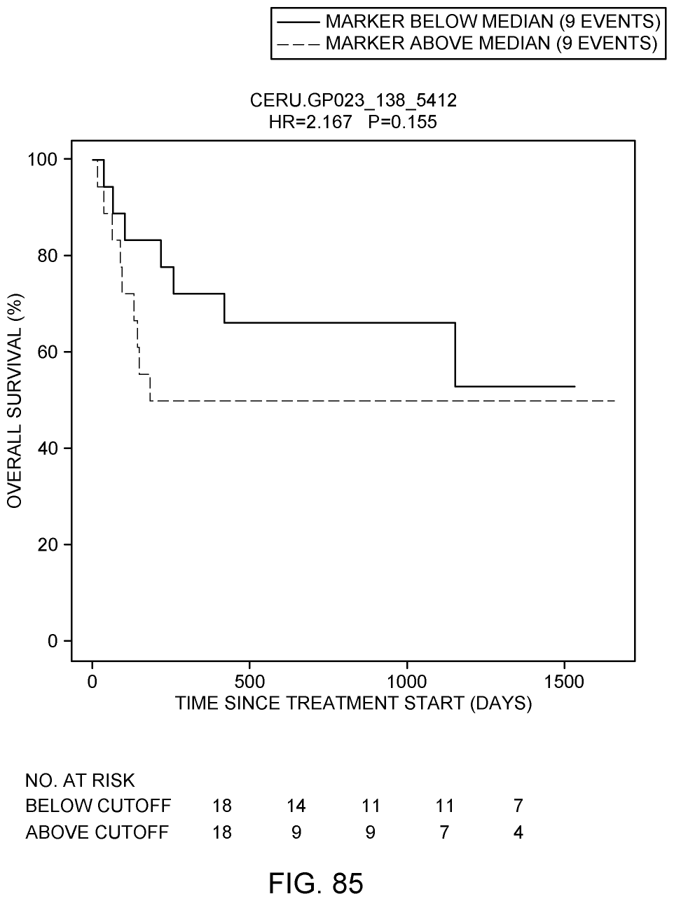

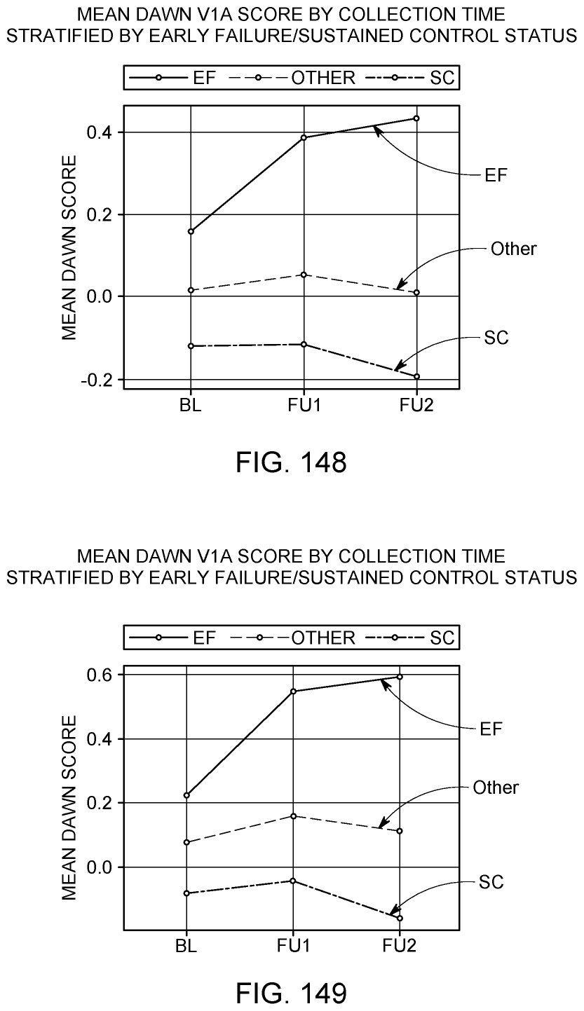

A diagnostic test was performed using a composite of two risk prediction models that were used to calculate two mean risk scores. In general, a higher risk score indicates that a subject is less likely to benefit from ICI therapy and a lower risk score indicates that a subject is more likely to benefit from ICI therapy. FIG. 148 illustrated the mean risk scores of the first prediction model, referred to as DAWN ViA, as a function of time after the ICI treatment. Similarly, FIG. 149 illustrated the mean risk scores of the second prediction model, referred to as DAWN V1B, as a function of time after the ICI treatment. In FIGS. 148 and 149, the term “Mean Dawn Score” was referred to as a mean risk score. With respect to the ICI treatment used in FIGS. 84 and 85, it represented either ipilimumab/nivolumab (ipi/nivo) treatment or pembrolizumab (pembro) treatment. The time points of the subjects for FIGS. 148 and 149 were a) baseline (BL) at or just before the start of ICI treatment, b) follow-up 1 (FU1) around 6 weeks post-BL, and c) follow-up 2 (FU2) around 6 months post-BL. The subjects of this Example were divided, based on the observed data, into 3 cohorts that were labeled as early failure (EF), sustained control (SC), and “Other”. The subjects of EF cohort progressed and died within a year of starting the ICI treatment. The subjects of the SC cohort remained alive and progression-free for at least three years after treatment start. The subjects of the “Other” cohort could not be labeled as either of EF or SC.

Referring back to FIGS. 148 and 149, the trends in the mean risk scores for the first prediction model and the second prediction model generally showed a similar pattern. The EF cohort showed an increase in mean risk scores in the first 6 weeks and remained elevated for the subsequent time point. The SC cohort showed a relatively low risk scores at BL along with relatively no change at FU1 and a modest decrease in mean risk scores at FU2. The “Other” cohort showed an intermediate mean risk score at BL followed by a relatively small change at FU1 and FU2. For reference, Table 84 showed the sample size for each cohort of EF, “Other”, and SC at the time points BL, FU1, and FU2 that correspond to FIGS. 148 and 149.

TABLE 84
Sample Size of the EF, “Other”, SC Cohorts and Various
Time Points in Accordance with FIGS. 148 and 149

| | BL | FU1 | FU2 |
|---|---|---|---|
| EF | 43 | 35 | 24 |
| Other | 70 | 65 | 68 |
| SC | 41 | 35 | 37 |

Table 85 showed the hazard ratios (HR) and p-values associated with the progression of malignant melanoma for both the first and second risk model when comparing various combination of time points (e.g., FU1 and BL; FU2 and BL; and FU2 and FU1). It should be noted that a positive difference in risk score between FU1 and BL was associated with disease progression.

TABLE 85
Cox proportional hazards regression modeling
based on progression of malignant melanoma
Progression of Malignant Melanoma

| | |
|---|---|
| Model V1A | Model V1B |

| | HR | P-value | HR | P-value |
|---|---|---|---|---|
| Δ(FU1, BL) | 6.735 | 0.001 | 3.140 | 0.002 |
| Δ(FU2, BL) | 3.649 | 0.130 | 1.836 | 0.237 |
| Δ(FU2, FU1) | 6.010 | 0.076 | 1.990 | 0.190 |

Table 86 showed the hazard ratios (HR) and p-values associated with the death of the subjects having malignant melanoma for both the first and second risk model when comparing various combination of time points (e.g., FU1 and BL, FU2 and BL, and FU2 and FU1). A positive difference in risk score between any two time points was associated with death. Cox proportional hazards regression modeling showed an increase of risk scores at follow-up time points and are associated with progression and death. It should be noted that regression analyses were adjusted for age and sex.

TABLE 86
Cox proportional hazards regression modeling
based on death of subject having malignant melanoma
Death of Subject Having Malignant Melanoma

| | |
|---|---|
| Model V1A | Model V1B |

| | HR | P-value | HR | P-value |
|---|---|---|---|---|
| Δ(FU1, BL) | 8.428 | <0.001 | 3.264 | <0.001 |
| Δ(FU2, BL) | 4.316 | <0.001 | 2.114 | 0.003 |
| Δ(FU2, FU1) | 5.606 | 0.001 | 2.106 | 0.016 |

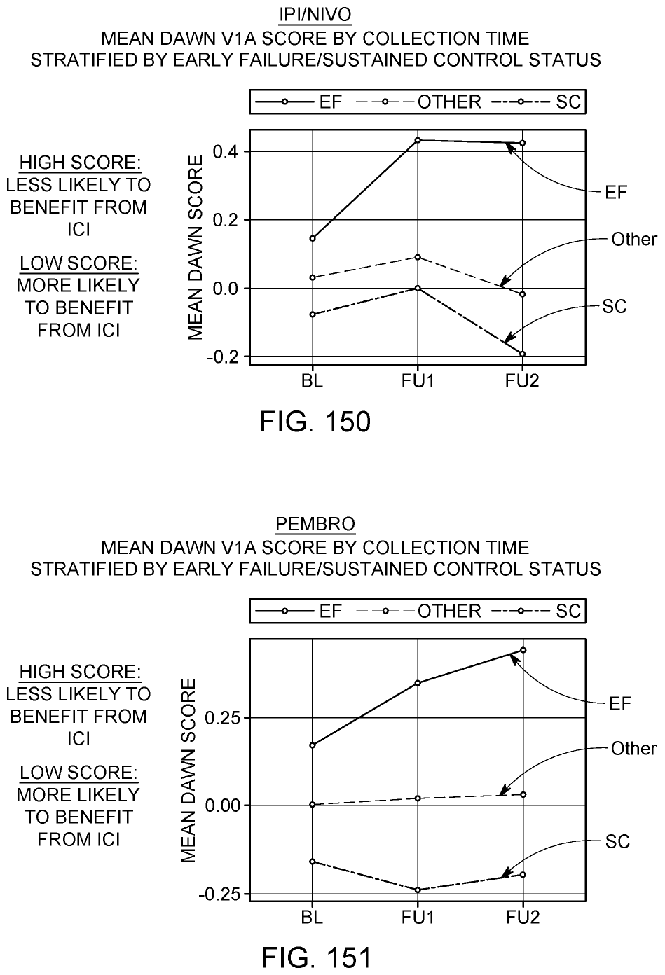

FIG. 150 illustrated the mean risk scores of the first prediction model (DAWN ViA) as a function of time after the specific ICI treatment combination therapy of ipilimumab (ipi) and nivolumab (nivo). Similarly, FIG. 151 illustrated the mean risk scores of the first prediction model (DAWN V1B), as a function of time after the specific ICI treatment therapy of pembrolizumab (pembro). Referring back to FIGS. 150 and 151, the trends in the mean risk scores for the ipi/nivo ICI treatment therapy and the pembro ICI treatment therapy generally showed a similar pattern. However, the ipi/nivo treatment group showed that the subjects who were part of the EF cohort had mean risk scores that showed a plateau after 6 weeks rather than a continued increase, while the “Other” and SC cohorts on ipi/nivo had a decrease in mean risk scores after six weeks (FU1). For reference, Table 87 showed the sample size for the ipi/nivo ICI treatment group that had the cohorts of EF, “Other”, and SC at the time points BL, FU1, and FU2. Table 88 showed the sample size for the pembro ICI treatment group that had the cohorts of EF, “Other”, and SC at the time points BL, FU1, and FU2. This Example showed that the diagnostic test can calculate a risk score for predicting the likelihood of benefit for ICI before therapy is administered. In addition to predicting the likelihood of ICI treatment benefit before therapy is administered, this diagnostic test can be used with subjects after they have started receiving ICI treatment (such as 6 weeks to 6 months after the start of ICI therapy). Alternatively, the risk score of can be measured at various time intervals during ICI treatment and used as a monitoring tool during ICI treatment so that a physician can make an informed decision about whether to continue, cease, or change the dose of the ICI treatment.

TABLE 87
Sample Size of the Ipi/nivo ICI treatment group
along with various cohorts and time points

| | BL | FU1 | FU2 |
|---|---|---|---|
| EF | 22 | 16 | 10 |
| Other | 33 | 31 | 28 |
| SC | 20 | 18 | 19 |

TABLE 88
Sample Size of the Pembro ICI treatment group
along with various cohorts and time points

| | BL | FU1 | FU2 |
|---|---|---|---|
| EF | 21 | 19 | 14 |
| Other | 37 | 34 | 40 |
| SC | 21 | 17 | 18 |

### 4. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1.-54. (canceled)

55. A method of detecting a presence of one of a plurality of states associated with fatty liver disease (FLD) progression in a biological sample, the method comprising:
receiving peptide structure data corresponding to a set of glycoproteins and/or non-glycosylated peptides in the biological sample obtained from a subject;
analyzing the peptide structure data using at least one supervised machine learning model to generate a disease indicator based on at least 2 peptide structures selected from a group of peptide structures identified in Table 1A; and
detecting the presence of a corresponding state of the plurality of states associated with the FLD progression in response to a determination that the disease indicator falls within a selected range associated with the corresponding state.

56. The method of claim 55, wherein a peptide structure of the at least 2 peptide structures comprises a non-glycosylated peptide or a glycopeptide structure defined by a peptide sequence and a glycan structure linked to the peptide sequence at a linking site of the peptide sequence, as identified in Table 1A, with the peptide sequence being one of SEQ ID NOS: 1-23 as defined in Table 1A.

57. The method of claim 55, wherein the at least one supervised machine learning model comprises a logistic regression model; or
wherein the at least one supervised machine learning model comprises a penalized multivariable logistic regression model.

58.-59. (canceled)

60. The method of claim 59, wherein the peptide structure data comprises quantification data; and
wherein the quantification data comprises at least one of an abundance, a relative abundance, a normalized abundance, a relative quantity, an adjusted quantity, a normalized quantity, a relative concentration, an adjusted concentration, or a normalized concentration.

61. (canceled)

62. The method of claim 55, wherein the disease indicator is a probability score.

63. The method of claim 55, further comprising:
generating a report that includes a diagnosis based on the corresponding state detected for the subject.

64. The method of claim 55, wherein the plurality of states includes a non-alcoholic steatohepatitis (NASH) state, a non-NASH state, or a stage of NASH state;
wherein the non-NASH state comprises at least one of a healthy state or a liver disease-free state; and
wherein the stage of the NASH state is early stage NASH or late stage NASH.

65.-66. (canceled)

67. The method of claim 55, wherein analyzing of the peptide structure data comprises: computing a peptide structure profile for the biological sample that identifies a weighted value for each peptide structure of the at least 2 peptide structures, wherein the weighted value for a peptide structure of the at least 2 peptide structures is a product of a quantification metric for the peptide structure identified from the peptide structure data and a weight coefficient for the peptide structure; and computing the disease indicator using the peptide structure profile.

68. The method of claim 55, wherein the corresponding state is non-alcoholic steatohepatitis (NASH) state and the selected range associated with the NASH state is between 0.05 and 0.4.

69. The method of claim 55, further comprising: creating a sample from the biological sample; and preparing the sample using reduction, alkylation, and enzymatic digestion to form a prepared sample that includes a set of peptide structures;
generating the peptide structure data from the prepared sample using multiple reaction monitoring mass spectrometry (MRM-MS); or
generating the peptide structure data from the prepared sample using liquid chromatography/mass spectrometry (LC/MS); and
wherein the biological sample comprises at least one of blood, serum, or plasma.

70.-73. (canceled)

74. The method of claim 55, further comprising: generating a treatment output based on the disease indicator.

75. The method of claim 74, wherein the treatment output comprises at least one of an identification of a treatment to treat the subject, a design for the treatment, a manufacturing plan for the treatment, or a treatment plan for administering the treatment.

76. A method of detecting a presence of one of a plurality of states associated with fatty liver disease (FLD) progression in a biological sample, the method comprising:
receiving peptide structure data corresponding to a set of glycoproteins and/or non-glycosylated peptides in the biological sample obtained from a subject;
analyzing the peptide structure data using at least one supervised machine learning model to generate a disease indicator based on at least 2 peptide structures selected from a group of peptide structures identified in Table 1B; and
detecting the presence of a corresponding state of the plurality of states associated with the FLD progression in response to a determination that the disease indicator falls within a selected range associated with the corresponding state.

77. The method of claim 76, wherein a peptide structure of the at least 2 peptide structures comprises a non-glycosylated peptide or a glycopeptide structure defined by a peptide sequence and a glycan structure linked to the peptide sequence at a linking site of the peptide sequence, as identified in Table 1B, with the peptide sequence being one of SEQ ID NOS: 1-11 as defined in Table 1B.

78. The method of claim 76, wherein the at least one supervised machine learning model comprises a logistic regression model; or
wherein the at least one supervised machine learning model comprises a penalized multivariable logistic regression model.

79.-80. (canceled)

81. The method of claim 80, wherein the peptide structure data comprises quantification data:
wherein the quantification data comprises at least one of an abundance, a relative abundance, a normalized abundance, a relative quantity, an adjusted quantity, a normalized quantity, a relative concentration, an adjusted concentration, or a normalized concentration; and
wherein the disease indicator is a probability score.

82.-83. (canceled)

84. The method of claim 76, further comprising:
generating a report that includes a diagnosis based on the corresponding state detected for the subject.

85. The method of claim 76, wherein the plurality of states includes one or more stages of a non-alcoholic steatohepatitis (NASH) state, or a non-NASH state that comprises at least one of a healthy state or a liver disease-free state; and
wherein the one or more stages of the NASH state includes a stage that is F1/F2 stage, or that is F3/F4 stage.

86.-88. (canceled)

89. A method of classifying a biological sample as corresponding to one of a plurality of states associated with fatty liver disease (FLD) progression, the method comprising:
training at least one supervised machine learning model using training data, wherein the training data comprises a plurality of peptide structure profiles for a plurality of training subjects and identifies a state of the plurality of states for each peptide structure profile of the plurality of peptide structure profiles;
receiving peptide structure data corresponding to a set of non-glycosylated peptides and/or glycopeptides in the biological sample obtained from a subject; inputting quantification data identified from the peptide structure data for a set of peptide structures into the supervised machine learning model that has been trained, wherein the set of peptide structures includes at least one peptide structure identified in Table 1A;
analyzing the quantification data using the supervised machine learning model to generate a score;
determining that the score falls within a selected range associated with a corresponding state of the plurality of states associated with the FLD progression; and
generating a diagnosis output that indicates that the biological sample evidences the corresponding state, wherein the plurality of states includes a non-alcoholic steatohepatitis (NASH) state or a non-NASH state.

90. The method of claim 89, further defined as:
training a supervised machine learning model using training data, wherein the training data comprises a plurality of peptide structure profiles for a plurality of training subjects and identifies a state of the plurality of states for each peptide structure profile of the plurality of peptide structure profiles;
receiving peptide structure data corresponding to a set of non-glycosylated peptides and/or glycopeptides in the biological sample obtained from a subject; inputting quantification data identified from the peptide structure data for a set of peptide structures into the supervised machine learning model that has been trained, wherein the set of peptide structures includes at least one peptide structure identified in Table 1B;
analyzing the quantification data using the supervised machine learning model to generate a score;
determining that the score falls within a selected range associated with a corresponding state of the plurality of states associated with the FLD progression; and
generating a diagnosis output that indicates that the biological sample evidences the corresponding state, wherein the plurality of states includes a non-alcoholic steatohepatitis (NASH) state or a non-NASH state.

91.-690. (canceled)
